(19) United States
(12) Patent Application Publication
(10) Pub. No.: US 2016/0185797 A1
(43) Pub. Date: Jun. 30, 2016

(54) ALKYNYL ALCOHOLS AND METHODS OF USE

(71) Applicant: Genentech, Inc., South San Francisco, CA (US)

(72) Inventors: Nicole Blaquiere, San Francisco, CA (US); Georgette Castanedo, Redwood City, CA (US); Jianwen A. Feng, Millbrae, CA (US); Baihua Hu, Plainsboro, NJ (US); Steven Staben, San Francisco, CA (US); Po-wai Yuen, Ann Arbor, MI (US); Guosheng Wu, Beijing (CN); Xingyu Lin, Beijing (CN); Jason Burch, Redwood City, CA (US)

(73) Assignee: Genentech, Inc., South San Francisco, CA (US)

(21) Appl. No.: 15/060,325

(22) Filed: Mar. 3, 2016

Related U.S. Application Data
(63) Continuation of application No. 14/466,176, filed on Aug. 22, 2014, now abandoned.

(30) Foreign Application Priority Data
Aug. 22, 2013 (CN) PCT/CN2013/000994
May 28, 2014 (CN) PCT/CN2014/078684
Jul. 22, 2014 (CN) PCT/CN2014/082696

(51) Int. Cl. C07D 495/04; C07D 403/10; C07D 403/14; C07D 491/107; C07D 409/14; C07D 417/14; C07D 487/04; C07D 401/14; C07D 471/04; C07D 413/10; C07D 405/10; C07D 401/10; C07D 405/14

(52) U.S. Cl. CPC C07D 495/04; C07D 401/10; C07D 403/10; C07D 403/14; C07D 491/107; C07D 409/14; C07D 405/14; C07D 487/04; C07D 401/14; C07D 471/04; C07D 413/10; C07D 405/10; C07D 417/14

(57) ABSTRACT

The invention relates to compounds of Formula (0):

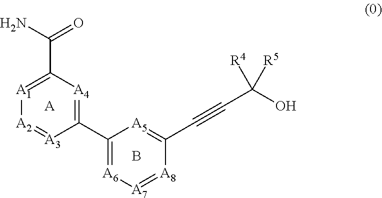

wherein A¹-A⁸, R⁴ and R⁵ each has the meaning as described herein. Compounds of Formula (0) and pharmaceutical compositions thereof are useful in the treatment of diseases and disorders in which undesired or over-activation of NF-kB signaling is observed.

### CROSS-REFERENCE TO RELATED APPLICATIONS

This application is a continuation of U.S. application Ser. No. 14/466,176, filed Aug. 22, 2014, which claims the benefit of priority under 35 U.S.C. §119(a) to: International Application No. PCT/CN2014/082696, filed Jul. 22, 2014; International Application No. PCT/CN2014/078684, filed May 28, 2014; and International Application No. PCT/CN2013/000994, filed Aug. 22, 2013, each of which is incorporated herein by reference in its entirety.

### FIELD OF THE INVENTION

The present invention relates to organic compounds useful for therapy or prophylaxis in a mammal, and in particular to inhibitors of NF-kB-inducing kinase (NIK) useful for treating cancer and inflammatory conditions, among others.

NF-kB inducing kinase (NIK) is also known as MAPK kinase kinase 14 (MAP3K14) and is a serine/threonine kinase and a member of the MAPK family. It was originally identified in a two-hybrid screen as a binding partner of TNF receptor (TNFR) associated factor 2 (TRAF2) [See, Malinin, N L, et al., Nature, 1997, 385:540-4]. Overexpression of NIK leads to the activation of NF-kB and dominant negative forms of NIK lacking kinase activity were able to inhibit NF-kB activation in response to TNF and IL-1 treatment. Thus, NIK has been identified as an important component of the NF-kB signaling pathway. Scientific research has shown that in blocking the NF-kB signaling pathway in cancer cells can cause such cells to stop proliferating, to die, or to become more sensitive to the action of other anti-cancer therapies. Additionally, research has shown that NF-kB controls the expression of many genes involved in inflammation and that NF-kB signaling is found to be chronically active in many inflammatory conditions, such as lupus (including systemic lupus erythematosus), rheumatoid arthritis, inflammatory bowel disease, arthritis, sepsis, gastritis and asthma, among others. Accordingly, organic compounds capable of inhibiting NIK and thereby inhibiting, weakening or lessening the undesired or over-activation of the NF-kB signaling pathway can have a therapeutic benefit for the treatment diseases and disorders for which such undesired or over-activation of NF-kB signaling is observed.

### SUMMARY OF THE INVENTION

Provided herein are compounds of Formula (0):

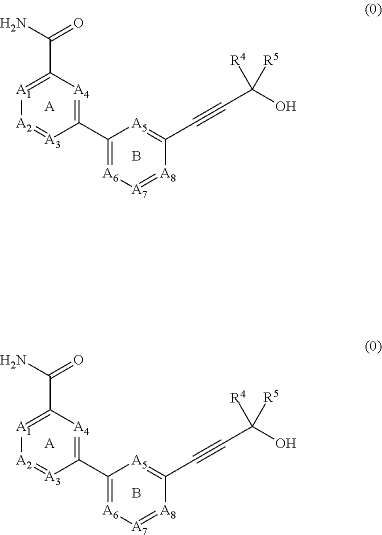

or a stereoisomer or salt thereof, wherein:

ring A is a monocycle or a fused bicycle;

A₁ is N or CR¹;

A₂ is N, NR² or CR²;

A₃ is N, NR³ or CR³;

A₄ is N or CH; and

one, two or three of A₁-A₄ is N, wherein:
R¹ is selected from the group consisting of H, halogen, OH, NR^{a}R^{b}, C₁-C₃ alkyl, C₃-C₇ cycloalkyl, C₁-C₃ alkoxy and 3-11 membered heterocyclyl, wherein each of R¹ is optionally substituted by F, OH, CN, SH, CH₃, or CF₃;
R² is selected from the group consisting of H, OH, NR^{a}R^{b}, C₁-C₆ alkyl optionally substituted by halogen, C₁-C₆ alkoxy, C₃-C₇ cycloalkyl, C₁-C₆ alkoxy, 3-6 membered heterocyclyloxy, phenyl and 3-11 membered heterocyclyl, wherein each of R² is optionally substituted by R^{c};
R³ is selected from the group consisting of H, C₁-C₆ alkyl, NR^{a}R^{b} and halogen; or
R¹ and R² taken together form a cyclic group selected from the group consisting of C₃-C₇ cycloalkyl, phenyl and 3-11 membered heterocyclyl, wherein the cyclic group is optionally substituted by R^{d}; or
R² and R³ taken together form a cyclic group selected from the group consisting of C₃-C₇ cycloalkyl, phenyl and a 3-11 membered heterocyclyl, wherein the cyclic group is optionally substituted by R^{d};

R⁴ is selected from the group consisting of C₁-C₆ alkyl, CH₂F and CH₂OH;

R⁵ is 3-11 membered heterocyclyl optionally substituted by R^{e}; or

R⁴ and R⁵ together form a C₃-C₁₁ cycloalkyl optionally substituted by R^{e} or a 3-11 membered heterocyclyl optionally substituted by R^{e};

one of A₅-A₈ is N and the remaining are CR⁶ or all are CR⁶;

R⁶, independently at each occurrence, is selected from the group consisting of H, F, Cl, NH₂, NHCH₃, N(CH₃)₂, OH, OCH₃, OCHF₂, OCH₂F, OCF₃, SH, SCH₃, SCHF₂, SCH₂F, CN, CH₃, CHF₂, CH₂F, CH₂OH, CF₃, NO₂ and N₃; or

two R⁶ are taken together to form a 5-6 membered heterocyclyl optionally substituted by R^{e};

R^{a} is selected from the group consisting of H and C₁-C₆ alkyl optionally substituted by C₁-C₃ alkoxy, F, OH, CN, SH, CH₃ or CF₃;

R^{b} is selected from the group consisting of H, C₁-C₆ alkyl, C₁-C₆ alkoxy, C₃-C₆ cycloalkyl, C(O)R^{g}, phenyl and 3-11 membered heterocyclyl wherein R^{b} may be optionally substituted by C₁-C₃ alkoxy, F, OH, CN, SH, CH₃, CF₃, or 3-6 membered heterocyclyl optionally substituted by R^{e};

R^{c} and R^{d} are each independently selected from the group consisting of halogen, —(X¹)₀₋₁—CN, —(X¹)₀₋₁—NO₂, —(X¹)₀₋₁—SF₅, —(X¹)₀₋₁—OH, —(X¹)₀₋₁—NH₂, —(X¹)₀₋₁—N(H)(R^{1a}), —(X¹)₀₋₁—N(R^{1b})(R^{1a}), —(X¹)₀₋₁—CF₃, C₁-C₆ alkyl, C₁-C₆ haloalkyl, C₁-C₆ heteroalkyl, C₁-C₆ alkoxy, C₁-C₆ alkylthio, oxo, —(X¹)₀₋₁—C₁-C₆ alkyl, —(X¹)₀₋₁—C₃-C₁₀ cycloalkyl, —(X¹)₀₋₁-3-11 membered heterocyclyl, —(X¹)₀₋₁—C₆-C₁₀ aryl, —C(═O)(X¹)₁—C₃-C₁₀ cycloalkyl, —C(═O)(X¹)₁-3-11 membered heterocyclyl, —(X¹)₀₋₁—C(═Y¹)N(H)(R^{1a}), —(X¹)₀₋₁—C(═Y¹)NH₂, —(X¹)₀₋₁—C(═Y¹)N(R^{1a})(R^{1b}), —(X¹)₀₋₁—C(═Y¹)OR^{1a}, —(X¹)₀₋₁—C(═Y¹)OH, —(X¹)₀₋₁—N(H)C(═Y¹)(R^{1a}), —(X¹)₀₋₁—N(R^{1b})C(═Y¹)(R^{1a}), —(X¹)₀₋₁—N(R^{1b})C(═Y¹)(H), —(X¹)₀₋₁—N(H)C(═Y¹)OR^{1a}, —(X¹)₀₋₁—N(R^{1b})C(═Y¹)OR^{1a}, —(X¹)₀₋₁—S(O)₁₋₂R^{1a}, —(X¹)₀₋₁—N(H)S(O)₁₋₂R^{1a}, —(X¹)₀₋₁—N(R^{1b})S(O)₁₋₂R^{1a}, —(X¹)₀₋₁—S(O)₀₋₁N(H)(R^{1a}), —(X¹)₀₋₁—S(O)₀₋₁N(R^{1b})(R^{1a}), —(X¹)₀₋₁—S(O)₀₋₁NH₂, —(X¹)₀₋₁—S(═O)(═NR^{1b})R^{1a}, —(X¹)₀₋₁—C(═Y¹)R^{1a}, —(X¹)₀₋₁—C(═Y¹)H, —(X¹)₀₋₁—C(═NOH)R^{1a}, —(X¹)₀₋₁—C(═NOR^{1b})R^{1a}, —(X¹)₀₋₁—NHC(═Y¹)N(H)(R^{1a}), —(X¹)₀₋₁—NHC(═Y¹)NH₂, —(X¹)₀₋₁—NHC(═Y¹)N(R^{1b})(R^{1a}), —(X¹)₀₋₁—N(R^{1a})C(═Y¹)N(H)(R^{1a}), —(X¹)₀₋₁—N(R^{1a})C(═Y¹)N(R^{1a})(R^{1b}), —(X¹)₀₋₁—N(R^{1a})C(═Y¹)NH₂, —(X¹)₀₋₁—OC(═Y¹)R^{1a}, —(X¹)₀₋₁—OC(═Y¹)H, —(X¹)₀₋₁—OC(═Y¹)OR^{1a}, —(X¹)₀₋₁—OP(═Y¹)(OR^{1a})(OR^{1b}), —(X¹)—SC(═Y¹)OR^{1a} and —(X¹)—SC(═Y¹)N(R^{1a})(R^{1b}) wherein X¹ is selected from the group consisting of C₁-C₆ alkylene, C₁-C₆ heteroalkylene, C₂-C₆ alkenylene, C₂-C₆ alkynylene, C₁-C₆ alkyleneoxy, C₃-C₇ cycloalkylene, 3-11 membered heterocyclylene and phenylene; R^{1a} and R^{1b} are each independently selected from the group consisting of C₁-C₆ alkyl, C₁-C₆ haloalkyl, C₁-C₆ heteroalkyl, C₃-C₇ cycloalkyl, (C₃-C₇ cycloalkylene)C₁-C₆ alkyl, 3-11 membered heterocyclyl, (3-11 membered heterocyclylene)C₁-C₆ alkyl, C₆ aryl, and (C₆-C₁₀ arylene)C₁-C₆ alkyl, or R^{1a} and R^{1b} when attached to the same nitrogen atom are optionally combined to form a 3-11 membered heterocyclyl comprising 0-3 additional heteroatoms selected from N, O and S; Y¹ is O, NR^{1c} or S wherein R^{1c} is H or C₁-C₆ alkyl; wherein any portion of an R^{c} or R^{d} substituent, including R^{1a}, R^{1b} and R^{1c}, at each occurrence is each independently further substituted by from 0 to 4 R^{f} substituents selected from the group consisting of halogen, CN, NO₂, SF₅, OH, NH₂, —N(C₁-C₆ alkyl)₂, —NH(C₁-C₆ alkyl), oxo, C₁-C₆ alkyl, C₁-C₆ hydroxyalkyl, C₁-C₆ heteroalkyl, C₁-C₆ alkoxy, C₁-C₆ alkylthio, C₃-C₇ cycloalkyl, 3-11 membered heterocyclyl, —C(═O)N(H)(C₁-C₆ alkyl), —C(═O)N(C₁-C₆ alkyl)₂, —C(═O)NH₂, —C(═O)OC₁—C₆ alkyl, —C(═O)OH, —N(H)C(═O)(C₁-C₆ alkyl), —N(C₁-C₆ alkyl)C(═O)(C₁-C₆ alkyl), —N(H)C(═O)OC₁—C₆ alkyl, —N(C₁-C₆ alkyl)C(═O)OC₁—C₆ alkyl, —S(O)₁₋₂C₁-C₆ alkyl, —N(H)S(O)₁₋₂C₁-C₆ alkyl, —N(C₁-C₆ alkyl)S(O)₁₋₂C₁-C₆ alkyl, —S(O)₀₋₁N(H)(C₁-C₆ alkyl), —S(O)₀₋₁N(C₁-C₆ alkyl)₂, —S(O)₀₋₁NH₂, —C(═O)C₁-C₆ alkyl, —C(═O)C₃-C₇ cycloalkyl, —C(═NOH)C₁-C₆ alkyl, —C(═NOC₁—C₆ alkyl)C₁-C₆ alkyl, —NHC(═O)N(H)(C₁-C₆ alkyl), —NHC(═O)N(C₁-C₆ alkyl)₂, —NHC(═O)NH₂, —N(C₁-C₆ alkyl)C(═O)N(H)(C₁-C₆ alkyl), —N(C₁-C₆ alkyl)C(═O)NH₂, —OC(═O)C₁-C₆ alkyl, —OC(═O)OC₁—C₆ alkyl, —OP(═O)(OC₁—C₆ alkyl)₂, —SC(═O)OC₁—C₆ alkyl and —SC(═O)N(C₁-C₆ alkyl)₂, wherein any alkyl portion of R^{f} is optionally substituted with halogen; and

R^{e} is selected from the group consisting of halogen, OH, C₁-C₆ alkyl and oxo; and

R^{g} is selected from the group consisting of C₁-C₆ alkyl and C₃-C₆ cycloalkyl.

In another aspect, the invention provides for pharmaceutical compositions comprising a compound of Formula (0) and a pharmaceutically acceptable carrier, diluent or excipient.

In another aspect, the invention provides for a compounds of Formula (0) or pharmaceutical compositions thereof for use in therapy. In another embodiment, the invention provides the use of a compound or pharmaceutical composition for the preparation of a medicament for the treatment of an inflammatory condition.

In another aspect, the inventions provides for compounds of Formula (0) and pharmaceutical compositions thereof for the treatment of diseases and disorders, including, cancer, inflammatory conditions, and autoimmune diseases, among others.

In another aspect, the invention provides for a method (or use) of compounds of Formula (0) or pharmaceutical compositions thereof in the treatment of diseases and disorders, such as, for example, cancer, inflammatory conditions, or autoimmune diseases, among others.

In another aspect, the invention provides for compounds of Formula (0) for the preparation of a medicament for the treatment of cancer, inflammatory conditions, or autoimmune diseases, among others.

In another aspect, the invention provides for compound intermediates useful in synthesis of compounds of Formula (0).

### DETAILED DESCRIPTION OF THE INVENTION

The invention provides for compounds of Formula (0), pharmaceutical compositions comprising compounds of Formula (0) and methods of using such compounds and compositions in treating diseases and disorders related to undesired or overactivation of the NF-kB signaling pathway, such as, for example, certain cancers and inflammatory conditions.

### DEFINITIONS

The term “alkyl” refers to a saturated linear or branched-chain monovalent hydrocarbon radical, wherein the alkyl radical may be optionally substituted independently with one or more substituents described herein. In one example, the alkyl radical is one to eighteen carbon atoms (C₁-C₁₈). In other examples, the alkyl radical is C₀-C₆, C₀-C₅, C₀-C₃, C₁-C₁₂, C₁-C₁₀, C₁-C₈, C₁-C₆, C₁-C₅, C₁-C₄, or C₁-C₃. C₀ alkyl refers to a bond. Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl and 1-octyl. In some embodiments, substituents for “optionally substituted alkyls” include one to six instances of F, Cl, Br, I, OH, SH, CN, NH₂, NO₂, N₃, COOH, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, or pyrimidinyl, wherein the alkyl, aryl and heterocyclic portions thereof may be optionally substituted.

The term “alkylene” by itself or as part of another substituent means a divalent radical derived from an alkane, as exemplified by —CH₂CH₂CH₂CH₂—. Typically, an alkyl (or alkylene) group will have from 1 to 12 carbon atoms, such as 1-8, 1-6 or 1-3 carbon atoms. “Alkenylene” and “alkynylene” refer to the unsaturated forms of “alkylene” having double or triple bonds, respectively, and typically have from 2 to 12 carbon atoms, such as 2-8, 2-6 or 2-3 carbon atoms. “Alkylene”, “alkenylene” and “alkynylene” groups may be optionally substituted.

The term “heteroalkyl” refers to a straight or branched chain monovalent hydrocarbon radical, consisting of the stated number of carbon atoms, or, if none are stated, up to 18 carbon atoms, and from one to five heteroatoms selected from the group consisting of O, N, Si and S, and wherein the nitrogen and sulfur atoms can optionally be oxidized and the nitrogen heteroatom can optionally be quaternized. In some embodiments, the heteroatom is selected from O, N and S, wherein the nitrogen and sulfur atoms can optionally be oxidized and the nitrogen heteroatom can optionally be quaternized. The heteroatom(s) can be placed at any interior position of the heteroalkyl group, including the position at which the alkyl group is attached to the remainder of the molecule (e.g., —O—CH₂—CH₃). Examples include —CH₂—CH₂—O—CH₃, —CH₂—CH₂—O—CF₃, —CH₂—CH₂—NH—CH₃, —CH₂—CH₂—N(CH₃)—CH₃, —CH₂—S—CH₂—CH₃, —S(O)—CH₃, —CH₂—CH₂—S(O)₂—CH₃, —Si(CH₃)₃, —CH₂—CH═N—OCH₃, and —OCF₃. Up to two heteroatoms can be consecutive, such as, for example, —CH₂—NH—OCH₃ and —CH₂—O—Si(CH₃)₃. Heteroalkyl groups can be optionally substituted. In some embodiments, substituents for “optionally substituted heteroalkyls” include one to four instances of F, Cl, Br, I, OH, SH, CN, NH₂, NO₂, N₃, COOH, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, and pyrimidinyl, wherein the alkyl, aryl and heterocyclic portions thereof may be optionally substituted.

The term “heteroalkylene” means a divalent radical derived from heteroalkyl, as exemplified by —CH₂CH₂SCH₂CH₂, —CH₂SCH₂CH₂NHCH₃ and —OCH₂CH₃. For heteroalkylene groups, heteroatoms can also occupy either or both of the chain termini (e.g., alkyleneoxy, alkylenedioxy, alkyleneamino, alkylenediamino, and the like). A heteroalkylene group may be optionally substituted.

“Cycloalkyl” refers to a non-aromatic, saturated or partially unsaturated hydrocarbon ring group wherein the cycloalkyl group may be optionally substituted with one or more substituents described herein. In one example, the cycloalkyl group is 3 to 12 carbon atoms (C₃-C₁₂). In other examples, cycloalkyl is C₃-C₆, C₃-C₈, C₃-C₁₀ or C₅-C₁₀. In other examples, the cycloalkyl group, as a monocycle, is C₃-C₈, C₃-C₆ or C₅-C₆. In another example, the cycloalkyl group, as a bicycle, is C₇-C₁₂. In another example, the cycloalkyl group, as a spiro system, is C₅-C₁₂. Examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Exemplary arrangements of bicyclic cycloalkyls having 7 to 12 ring atoms include, but are not limited to, [4,4], [4,5], [5,5], [5,6] or [6,6] ring systems. Exemplary bridged bicyclic cycloalkyls include, but are not limited to, bicyclo[4.1.0]heptane, bicycle[3.1.1]heptane, bicyclo[2.2.1]heptane, bicyclo[2.2.2]octane and bicyclo[3.2.2]nonane. Examples of spiro cycloalkyl include, spiro[2.2]pentane, spiro[2.3]hexane, spiro[2.4]heptane, spiro[2.5]octane and spiro[4.5]decane. In some embodiments, substituents for “optionally substituted cycloalkyls” include one to four instances of F, Cl, Br, I, OH, SH, CN, NH₂, NO₂, N₃, COOH, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, and pyrimidinyl, wherein the alkyl, aryl and heterocyclic portions thereof may be optionally substituted.

The term “cycloalkylene” means a divalent radical derived from a cycloalkyl group. A cycloalkylene group may be optionally substituted.

“Heterocyclic group”, “heterocyclic”, “heterocycle”, “heterocyclyl”, or “heterocyclo” are used interchangeably and refer to any monocyclic, bicyclic, or spiro, saturated or unsaturated, aromatic (heteroaryl) or non-aromatic (e.g., heterocycloalkyl), ring system, where the ring atoms are carbon, and at least one atom in the ring or ring system is a heteroatom selected from nitrogen, sulfur or oxygen. If any ring atom of a cyclic system is a heteroatom, that system is a heterocycle, regardless of the point of attachment of the cyclic system to the rest of the molecule. In one example, heterocyclyl includes 3-11 ring atoms (“members”, that is, a 3-11 membered heterocycle) and includes monocycles, bicycles, and spiro ring systems, wherein the ring atoms are carbon, and at least one atom in the ring or ring system is a heteroatom selected from nitrogen, sulfur or oxygen. In one example, heterocyclyl includes 1 to 4 heteroatoms. In another example, heterocyclyl includes 3- to 7-membered monocycles having one or more heteroatoms selected from nitrogen, sulfur or oxygen. In another example, heterocyclyl includes 4- to 6-membered monocycles having one or more heteroatoms selected from nitrogen, sulfur or oxygen. In another example, heterocyclyl includes 3-membered monocycles. In another example, heterocyclyl includes 4-membered monocycles. In another example, heterocyclyl includes 5-6-membered monocycles. In one example, the heterocyclyl group includes 0 to 3 double bonds. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). In another example, heterocyclyl includes 3- to 9-membered spiro cycles having one or more heteroatoms selected from nitrogen, sulfur or oxygen. Example heterocycles are oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidinyl, piperazinyl, isoquinolinyl, tetrahydroisoquinolinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocycles containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocycles containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Example benzo-fused 5-membered heterocycles are benzoxazol-2-yl, benzthiazol-2-yl and benzimidazol-2-yl. Example 6-membered heterocycles contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are other example heterocycle groups. Heterocycles may be optionally substituted. For example, substituents for “optionally substituted heterocycles” include one to six instances of F, Cl, Br, I, OH, SH, CN, NH₂, NO₂, N₃, COOH, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, and pyrimidinyl, wherein the alkyl, aryl and heterocyclic portions thereof may be optionally substituted.

The term “heterocyclylene” means a divalent radical derived from a heterocyclyl group. A heterocyclylene group may be optionally substituted.

“Heteroaryl” refers to any mono-, bi-, or tricyclic ring system where at least one ring is a 5- or 6-membered aromatic ring containing from 1 to 4 heteroatoms selected from nitrogen, oxygen, and sulfur, and in an example embodiment, at least one heteroatom is nitrogen. See, for example, Lang's Handbook of Chemistry (Dean, J. A., ed.) 13^{th} ed. Table 7-2 [1985]. Included in the definition are any bicyclic groups where any of the above heteroaryl rings are fused to an aryl ring, wherein the aryl ring or the heteroaryl ring is joined to the remainder of the molecule. In one embodiment, heteroaryl includes 4-6 membered monocyclic aromatic groups where one or more ring atoms is nitrogen, sulfur or oxygen. In another embodiment, heteroaryl includes 5-6 membered monocyclic aromatic groups where one or more ring atoms is nitrogen, sulfur or oxygen. Example heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, imidazol[1,2-a]pyrimidinyl and purinyl, as well as benzo-fused derivatives, for example benzoxazolyl, benzofuryl, benzothiazolyl, benzothiadiazolyl, benzotriazolyl, benzoimidazolyl and indolyl. Heteroaryl groups can be optionally substituted. In some embodiments, substituents for “optionally substituted heteroaryls” include one to six instances of F, Cl, Br, I, OH, SH, CN, NH₂, NO₂, N₃, COOH, methyl, ethyl, propyl, iso-propyl, butyl, isobutyl, cyclopropyl, methoxy, ethoxy, propoxy, oxo, trifluoromethyl, difluoromethyl, sulfonylamino, methanesulfonylamino, SO, SO₂, phenyl, piperidinyl, piperizinyl, and pyrimidinyl, wherein the alkyl, aryl and heterocyclic portions thereof may be optionally substituted.

In particular embodiments, a heterocyclyl group is attached at a carbon atom of the heterocyclyl group. By way of example, carbon bonded heterocyclyl groups include bonding arrangements at position 2, 3, 4, 5, or 6 of a pyridine ring, position 3, 4, 5, or 6 of a pyridazine, position 2, 4, 5, or 6 of a pyrimidine ring, position 2, 3, 5, or 6 of a pyrazine ring, position 2, 3, 4, or 5 of a furan, tetrahydrofuran, thiofuran, thiophene, pyrrole or tetrahydropyrrole ring, position 2, 4, or 5 of an oxazole, imidazole or thiazole ring, position 3, 4, or 5 of an isoxazole, pyrazole, or isothiazole ring, position 2 or 3 of an aziridine ring, position 2, 3, or 4 of an azetidine ring, position 2, 3, 4, 5, 6, 7, or 8 of a quinoline ring or position 1, 3, 4, 5, 6, 7, or 8 of an isoquinoline ring.

In certain embodiments, the heterocyclyl group is N-attached. By way of example, the nitrogen bonded heterocyclyl or heteroaryl group include bonding arrangements at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of an isoindole, or isoindoline, position 4 of a morpholine, and position 9 of a carbazole, or β-carboline.

The term “alkoxy” refers to those alkyl groups attached to the remainder of the molecule via an oxygen atom. Non-limiting examples include methoxy, ethoxy and propoxy. Alkoxy groups may be optionally substituted, such as by halogen.

The term “alkylthio” refers to those alkyl groups attached to the remainder of the molecule via an sulfur atom. Non-limiting examples include —SCH₃, —SCH₂CH₃ and —SCH₂CH₂CH₃. Alkylthio groups may be optionally substituted, such as by halogen.

The terms “halo” or “halogen,” by themselves or as part of another substituent, mean, unless otherwise stated, a fluorine, chlorine, bromine, or iodine atom. The term “haloalkyl” is meant to include both an “alkyl” and a “haloalkyl” substituent. Additionally, the term “haloalkyl,” is meant to include monohaloalkyl and polyhaloalkyl.

The term “oxo” refers to ═O or (═O)₂.

The term “aryl” means, unless otherwise stated, a polyunsaturated, typically aromatic, hydrocarbon ring radical, which can be a single ring or multiple rings (up to three rings) which are fused together and having the stated number of aryl ring atoms. An aryl group can be optionally substituted.

A “phenylene” group refers to a divalent radical derived from a phenyl group. A phenylene group may be optionally substituted.

“Optionally substituted” unless otherwise specified means that a group may be unsubstituted or substituted by one or more (e.g., 0, 1, 2, 3, 4, or 5 or more) of the substituents listed for that group in which said substituents may be the same or different. That is, an optionally substituted substituent is independent at each occurrence. In an embodiment an optionally substituted group has 1 substituent. In another embodiment an optionally substituted group has 2 substituents. In another embodiment an optionally substituted group has 3 substituents. In another embodiment an optionally substituted group has 4 substituents.

Optional substituents for alkyl and cycloalkyl can be a variety of groups including, but not limited to, halogen, oxo, CN, NO₂, N₃, OR′, perfluoro-C₁₋₄ alkoxy, unsubstituted cycloalkyl, unsubstituted aryl (e.g., phenyl), unsubstituted heterocyclyl, NR′R″, SR′, SiR′R″R′″, OC(O)R′, C(O)R′, CO₂R′, CONR′R″, OC(O)NR′R″, NR″C(O)R′, NR′″C(O)NR′R″, NR″C(O)₂R′, S(O)₂R′, S(O)₂NR′R″, NR′S(O)₂R″, NR″′S(O)₂NR′R″, amidino, guanidine, (CH₂)₁₋₄OR′, (CH₂)₁₋₄NR′R″, (CH₂)₁₋₄SR′, (CH₂)₁₋₄SiR′R″R′″, (CH₂)₁₋₄OC(O)R′, (CH₂)₁₋₄C(O)R′, (CH₂)₁₋₄CO₂R′, and (CH₂)₁₋₄CONR′R″, or combinations thereof, in a number ranging from zero to (2m′+1), where m′ is the total number of carbon atoms in such radical. R′, R″ and R′″ each independently refer to groups including, for example, hydrogen; unsubstituted C₁₋₆ alkyl; unsubstituted heteroalkyl; unsubstituted aryl; aryl substituted with 1-3 halogens, unsubstituted C₁-C₆ alkyl, C₁-C₆ alkoxy or C₁-C₆ thioalkoxy groups, unsubstituted aryl-C₁-C₄ alkyl groups, and unsubstituted heteroaryl. When R′ and R″ are attached to the same nitrogen atom, they can be combined with the nitrogen atom to form a 3-, 4-, 5-, 6-, or 7-membered ring wherein a ring atom is optionally substituted with N, O or S. For example, NR′R″ is meant to include 1-pyrrolidinyl and 4-morpholinyl.

Similarly, optional substituents for the aryl and heterocyclyl groups are varied. In some embodiments, substituents for aryl and heterocyclyl groups are selected from the group including, but not limited to, halogen, OR′, OC(O)R′, NR′R″, SR′, R′, CN, NO₂, CO₂R′, CONR′R″, C(O)R′, OC(O)NR′R″, NR″C(O)R′, NR″C(O)₂R′, NR′C(O)NR″R′″, S(O)R′, S(O)₂R′, S(O)₂NR′R″, NR'S(O)₂R″, N₃, perfluoro-C₁-C₄alkoxy, perfluoro-C₁-C₄alkoxy, (CH₂)₁₋₄OR′, (CH₂)₁₋₄NR′R″, (CH₂)₁₋₄SR′, (CH₂)₁₋₄SiR′R″R′″, (CH₂)₁₋₄OC(O)R′, (CH₂)₁₋₄C(O)R′, (CH₂)₁₋₄CO₂R′, (CH₂)₁₋₄CONR′R″, or combinations thereof, in a number ranging from zero to the total number of open valences on the aromatic ring system; and where R′, R″ and R′″ are independently selected from hydrogen, C₁-C₆ alkyl, C₃-C₆ cycloalkyl, C₂-C₆ alkenyl, C₂-C₆ alkynyl, unsubstituted aryl, and unsubstituted heteroaryl. Other suitable substituents include each of the above aryl substituents attached to a ring atom by an alkylene tether of from 1-4 carbon atoms.

As used herein, the term “heteroatom” is meant to include oxygen (O), nitrogen (N), sulfur (S) and silicon (Si). In some embodiments, heteroatom refers to O, N or S. In some embodiments, heteroatom refers to O or N.

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers can separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined. Unless otherwise specified, if solid wedges or dashed lines are used, relative stereochemistry is intended. If a discrepancy exists between a structure and its name, the structure governs.

As used herein, the term “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein, the term “mammal” includes, but is not limited to, humans, mice, rats, guinea pigs, monkeys, dogs, cats, horses, cows, pigs, and sheep.

A “subject,” “individual,” or “patient” is a vertebrate. In certain embodiments, the vertebrate is a mammal. A subject, individual or patient may be in need of a compound of the present invention.

As used herein, the term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, J. Pharm. Sci., 1977, 66, 1-19). Certain specific compounds of the present invention contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. As used herein the term “prodrug” refers to those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Prodrugs of the invention include compounds wherein an amino acid residue, or a polypeptide chain of two or more (e.g., two, three or four) amino acid residues, is covalently joined through an amide or ester bond to a free amino, hydroxy or carboxylic acid group of a compound of the present invention. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, citrulline, homocysteine, homoserine, methyl-alanine, para-benzoylphenylalanine, phenylglycine, propargylglycine, sarcosine, methionine sulfone and tert-butylglycine.

Additional types of prodrugs are also encompassed. For instance, a free carboxyl group of a compound of the invention can be derivatized as an amide or alkyl ester. As another example, compounds of this invention comprising free hydroxy groups can be derivatized as prodrugs by converting the hydroxy group into a group such as, but not limited to, a phosphate ester, hemisuccinate, dimethylaminoacetate, or phosphoryloxymethyloxycarbonyl group, as outlined in Fleisher, D. et al., (1996) Improved oral drug delivery: solubility limitations overcome by the use of prodrugs Advanced Drug Delivery Reviews, 19:115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group can be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., (1996), 39:10. More specific examples include replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, alpha-amino(C₁₋₄)alkanoyl, arylacyl and alpha-aminoacyl, or alpha-aminoacyl-alpha-aminoacyl, where each alpha-aminoacyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77: 285 (1988); and e) N. Kakeya, et al., Chem. Pharm. Bull., 32:692 (1984), each of which is specifically incorporated herein by reference.

Additionally, the present invention provides for metabolites of compounds of the invention. As used herein, a “metabolite” refers to a product produced through metabolism in the body of a specified compound or salt thereof. Such products can result for example from the oxidation, reduction, hydrolysis, amidation, deamidation, esterification, deesterification, enzymatic cleavage, and the like, of the administered compound.

Metabolite products typically are identified by preparing a radiolabelled (e.g., ¹⁴C or ³H) isotope of a compound of the invention, administering it in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

Certain compounds of the present invention can exist in unsolvated forms as well as solvated forms, including hydrated forms. Compounds of the present invention may exist in multiple crystalline or amorphous forms. In general, all physical forms are intended to be within the scope of the present invention.

The compounds of the present invention can also contain unnatural proportions of atomic isotopes at one or more of the atoms that constitute such compounds. For example, the present invention also embraces isotopically-labeled variants of the present invention which are identical to those recited herein, but for the fact that one or more atoms are replace by an atom having the atomic mass or mass number different from the predominant atomic mass or mass number usually found in nature for the atom. All isotopes of any particular atom or element as specified are contemplated within the scope of the compounds of the invention, and their uses. Exemplary isotopes that can be incorporated in to compounds of the invention include istopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³₂P, ³³P, ³⁵S, ¹⁸F, ³⁶Cl, ¹²³I and ¹²⁵I. Certain isotopically labeled compounds of the present invention (e.g., those labeled with ³H or ¹⁴C) are useful in compound or substrate tissue distribution assays. Tritiated (³H) and carbon-14 (¹⁴C) isotopes are useful for their ease of preparation and detectability. Further substituteion with heavier isotopes such as deuterium (i.e., ²H) may afford certain therapeutic advantages resuting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine substrate receptor occupancy. Isotopically labeled compounds of the present inventions can generally be prepared by following procedures analogous to those disclosed in the Schemes and Examples herein, by substituting an isotopically labeled reagent for a non-isotopically labeled reagent. One non-limiting example of an isotopically substituted moiety is the following:

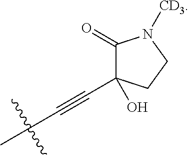 [SMILES: *C#CC1(O)CCN(C([2H])([2H])[2H])C1=O]

The terms “compound(s) of this invention,” and “compound(s) of the present invention” and the like, unless otherwise indicated, include compounds of Formula (0) and stereoisomers (including atropisomers), geometric isomers, tautomers, solvates, metabolites, isotopes, salts (e.g., pharmaceutically acceptable salts), and prodrugs thereof. In some embodiments, solvates, metabolites, isotopes or prodrugs are excluded, or any combination thereof

“Treatment” (and variations such as “treat” or “treating”) refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, stabilized (i.e., not worsening) state of disease, decreasing the rate of disease progression, amelioration or palliation of the disease state, prolonging survival as compared to expected survival if not receiving treatment and remission or improved prognosis. In some embodiments, compounds of the invention are used to delay development of a disease or disorder or to slow the progression of a disease or disorder. Those in need of treatment include those already with the condition or disorder as well as those prone to have the condition or disorder, (for example, through a genetic mutation) or those in which the condition or disorder is to be prevented. In some embodiments, prophylaxis is excluded from the definition of “treatment.”

The phrase “therapeutically effective amount” or “effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. For cancer therapy, efficacy can, for example, be measured by assessing the time to disease progression (TTP) or determining the response rate (RR). In the case of immunological disease, the therapeutically effective amount is an amount sufficient to decrease or alleviate an allergic disorder, the symptoms of an autoimmune or inflammatory condition (e.g., psoriasis or inflammatory bowel disease), or the symptoms of an acute inflammatory reaction (e.g. asthma). In some embodiments, a therapeutically effective amount is an amount of a chemical entity described herein sufficient to significantly decrease the activity or number of B-cells.

The terms “inhibiting” and “reducing,” or any variation of these terms, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity (e.g., NIK activity) compared to normal.

The term “bioavailability” refers to the systemic availability (i.e., blood/plasma levels) of a given amount of drug administered to a patient. Bioavailability is an absolute term that indicates measurement of both the time (rate) and total amount (extent) of drug that reaches the general circulation from an administered dosage form.

“Inflammatory condition” as used herein refers to any disease, disorder, or syndrome in which an excessive or unregulated inflammatory response leads to excessive inflammatory symptoms, host tissue damage, or loss of tissue function.

“Inflammation” as used herein refers to a localized, protective response elicited by injury or destruction of tissues, which serves to destroy, dilute, or wall off (sequester) both the injurious agent and the injured tissue. Inflammation is notably associated with influx of leukocytes or neutrophil chemotaxis. Inflammation can result from infection with pathogenic organisms and viruses and from noninfectious means such as trauma or reperfusion following myocardial infarction or stroke, immune response to foreign antigen, and autoimmune responses.

The terms “cancer” and “cancerous” refer to or describe the physiological condition in mammals that is typically characterized by unregulated cell growth or proliferation. A “tumor” comprises one or more cancerous cells. Examples of cancer include, but are not limited to, carcinoma, lymphoma, blastoma, sarcoma, and leukemia or lymphoid malignancies.

“Autoimmune disease” as used herein refers to any group of disorders in which tissue injury is associated with humoral or cell-mediated responses to the body's own constituents.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

The use of the term “or” is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device or method being employed to determine the value.

As used herein, “a” or “an” means one or more, unless clearly indicated otherwise. As used herein, “another” means at least a second or more.

Headings used herein are intended only for organizational purposes.

Inhibitors of NIK

One aspect of the invention provides compounds of Formula (0):

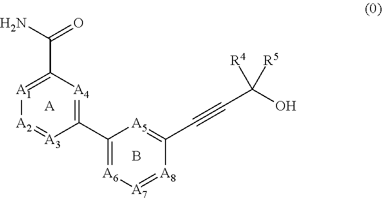

or a stereoisomer or salt thereof, wherein:

ring A is a monocycle or a fused bicycle;

A₁ is N or CR¹;

A₂ is N, NR² or CR²;

A₃ is N, NR³ or CR³;

A₄ is N or CH; and

one, two or three of A₁-A₄ is N, wherein:
R¹ is selected from the group consisting of H, halogen, OH, NR^{a}R^{b}, C₁-C₃ alkyl, C₃-C₇ cycloalkyl, C₁-C₃ alkoxy and 3-11 membered heterocyclyl, wherein each of R¹ is optionally substituted by F, OH, CN, SH, CH₃, or CF₃;
R² is selected from the group consisting of H, OH, NR^{a}R^{b}, C₁-C₆ alkyl, C₃-C₇ cycloalkyl, C₁-C₆ alkoxy, 3-6 membered heterocyclyloxy, phenyl and 3-11 membered heterocyclyl, wherein each of R² is optionally substituted by R^{c};
R³ is selected from the group consisting of H, C₁-C₆ alkyl optionally substituted by halogen, C₁-C₆ alkoxy, NR^{a}R^{b} and halogen; or
R¹ and R² taken together form a cyclic group selected from the group consisting of C₃-C₇ cycloalkyl, phenyl and 3-11 membered heterocyclyl, wherein the cyclic group is optionally substituted by R^{d}; or
R² and R³ taken together form a cyclic group selected from the group consisting of C₃-C₇ cycloalkyl, phenyl and a 3-11 membered heterocyclyl, wherein the cyclic group is optionally substituted by R^{d};

R⁴ is selected from the group consisting of C₁-C₆ alkyl, CH₂F and CH₂OH;

R⁵ is 3-11 membered heterocyclyl optionally substituted by R^{e}; or

R⁴ and R⁵ together form a C₃-C₁₁ cycloalkyl optionally substituted by R^{e} or a 3-11 membered heterocyclyl optionally substituted by R^{e};

one of A₅-A₈ is N and the remaining are CR⁶ or all are CR⁶;

R⁶, independently at each occurrence, is selected from the group consisting of H, F, Cl, NH₂, NHCH₃, N(CH₃)₂, OH, OCH₃, OCHF₂, OCH₂F, OCF₃, SH, SCH₃, SCHF₂, SCH₂F, CN, CH₃, CHF₂, CH₂F, CH₂OH, CF₃, NO₂ and N₃; or

two R⁶ are taken together to form a 5-6 membered heterocyclyl optionally substituted by R^{e};

R^{a} is selected from the group consisting of H and C₁-C₆ alkyl optionally substituted by C₁-C₃ alkoxy, F, OH, CN, SH, CH₃ or CF₃;

R^{b} is selected from the group consisting of H, C₁-C₆ alkyl, C₁-C₆ alkoxy, C₃-C₆ cycloalkyl, C(O)R^{g}, phenyl and 3-11 membered heterocyclyl wherein R^{b} may be optionally substituted by C₁-C₃ alkoxy, F, OH, CN, SH, CH₃, CF₃, or 3-6 membered heterocyclyl optionally substituted by R^{e};

R^{c} and R^{d} are each independently selected from the group consisting of halogen, —(X¹)₀₋₁—CN, —(X¹)₀₋₁—NO₂, —(X¹)₀₋₁—SF₅, —(X¹)₀₋₁—OH, —(X¹)₀₋₁—NH₂, —(X¹)₀₋₁—N(H)(R^{1a}), —(X¹)₀₋₁—N(R^{1b})(R^{1a}), —(X¹)₀₋₁—CF₃, CF₃, C₁-C₆ alkyl, C₁-C₆ haloalkyl, C₁-C₆ heteroalkyl, C₁-C₆ alkoxy, C₁-C₆ alkylthio, oxo, —(X¹)₀₋₁—C₁-C₆ alkyl, —(X¹)₀₋₁—C₃-C₁₀ cycloalkyl, —(X¹)₀₋₁-3-11 membered heterocyclyl, —(X¹)₀₋₁—C₆-C₁₀ aryl, —C(═O)(X¹)₁—C₃-C₁₀ cycloalkyl, —C(═O)(X¹)₁-3-11 membered heterocyclyl, —(X¹)₀₋₁—C(═Y¹)N(H)(R^{1a}), —(X¹)₀₋₁—C(═Y¹)NH₂, —(X¹)₀₋₁—C(═Y¹)N(R^{1a})(R^{1b}), —(X¹)₀₋₁—C(═Y¹)OR^{1a}, —(X¹)₀₋₁—C(═Y¹)OH, —(X¹)₀₋₁—N(H)C(═Y¹)(R^{1a}), —(X¹)₀₋₁—N(R^{1b})C(═Y¹)(R^{1a}), —(X¹)₀₋₁—N(R^{1b})C(═Y¹)(H), —(X¹)₀₋₁—N(H)C(═Y¹)OR^{1a}, —(X¹)₀₋₁—N(R^{1b})C(═Y¹)OR^{1a}, —(X¹)₀₋₁—S(O)₁₋₂R^{1a}, —(X¹)₀₋₁—N(H)S(O)₁₋₂R^{1a}, —(X¹)₀₋₁—N(R^{1b})S(O)₁₋₂R^{1a}, —(X¹)₀₋₁—S(O)₀₋₁N(H)(R^{1a}), —(X¹)₀₋₁—S(O)₀₋₁N(R^{1b})(R^{1a}), —(X¹)₀₋₁—S(O)₀₋₁NH₂, —(X¹)₀₋₁—S(═O)(═NR^{1b})R^{1a}, —(X¹)₀₋₁—C(═Y¹)R^{1a}, —(X¹)₀₋₁—C(═Y¹)H, —(X¹)₀₋₁—C(═NOH)R^{1a}, —(X¹)₀₋₁—C(═NOR^{1b})R^{1a}, —(X¹)₀₋₁—NHC(═Y¹)N(H)(R^{1a}), —(X¹)₀₋₁—NHC(═Y¹)NH₂, —(X¹)₀₋₁—NHC(═Y¹)N(R^{1b})(R^{1a}), —(X¹)₀₋₁—N(R^{1a})C(═Y¹)N(H)(R^{1a}), —(X¹)₀₋₁—N(R^{1a})C(═Y¹)N(R^{1a})(R^{1b}), —(X¹)₀₋₁—N(R^{1a})C(═Y¹)NH₂, —(X¹)₀₋₁—OC(═Y¹)R^{1a}, —(X¹)₀₋₁—OC(═Y¹)H, —(X¹)₀₋₁—OC(═Y¹)OR^{1a}, —(X¹)₀₋₁—OP(═Y¹)(OR^{1a})(OR^{1b}), —(X¹)—SC(═Y¹)OR^{1a} and —(X¹)—SC(═Y¹)N(R^{1a})(R^{1b}) wherein X¹ is selected from the group consisting of C₁-C₆ alkylene, C₁-C₆ heteroalkylene, C₂-C₆ alkenylene, C₂-C₆ alkynylene, C₁-C₆ alkyleneoxy, C₃-C₇ cycloalkylene, 3-11 membered heterocyclylene and phenylene; R^{1a} and R^{1b} are each independently selected from the group consisting of C₁-C₆ alkyl, C₁-C₆ haloalkyl, C₁-C₆ heteroalkyl, C₃-C₇ cycloalkyl, (C₃-C₇ cycloalkylene)C₁-C₆ alkyl, 3-11 membered heterocyclyl, (3-11 membered heterocyclylene)C₁-C₆ alkyl, C₆ aryl, and (C₆-C₁₀ arylene)C₁-C₆ alkyl, or R^{1a} and R^{1b} when attached to the same nitrogen atom are optionally combined to form a 3-11 membered heterocyclyl comprising 0-3 additional heteroatoms selected from N, O and S; Y¹ is O, NR^{1c} or S wherein R^{1c} is H or C₁-C₆ alkyl; wherein any portion of an R^{c} or R^{d} substituent, including R^{1a}, R^{1b} and R^{1c}, at each occurrence is each independently further substituted by from 0 to 4 R^{f} substituents selected from the group consisting of halogen, CN, NO₂, SF₅, OH, NH₂, —N(C₁-C₆ alkyl)₂, —NH(C₁-C₆ alkyl), oxo, C₁-C₆ alkyl, C₁-C₆ hydroxyalkyl, C₁-C₆ heteroalkyl, C₁-C₆ alkoxy, C₁-C₆ alkylthio, C₃-C₇ cycloalkyl, 3-11 membered heterocyclyl, —C(═O)N(H)(C₁-C₆ alkyl), —C(═O)N(C₁-C₆ alkyl)₂, —C(═O)NH₂, —C(═O)OC₁—C₆ alkyl, —C(═O)OH, —N(H)C(═O)(C₁-C₆ alkyl), —N(C₁-C₆ alkyl)C(═O)(C₁-C₆ alkyl), —N(H)C(═O)OC₁—C₆ alkyl, —N(C₁-C₆ alkyl)C(═O)OC₁—C₆ alkyl, —S(O)₁₋₂C₁-C₆ alkyl, —N(H)S(O)₁₋₂C₁-C₆ alkyl, —N(C₁-C₆ alkyl)S(O)₁₋₂C₁-C₆ alkyl, —S(O)₀₋₁N(H)(C₁-C₆ alkyl), —S(O)₀₋₁N(C₁-C₆ alkyl)₂, —S(O)₀₋₁NH₂, —C(═O)C₁-C₆ alkyl, —C(═O)C₃-C₇ cycloalkyl, —C(═NOH)C₁-C₆ alkyl, —C(═NOC₁—C₆ alkyl)C₁-C₆ alkyl, —NHC(═O)N(H)(C₁-C₆ alkyl), —NHC(═O)N(C₁-C₆ alkyl)₂, —NHC(═O)NH₂, —N(C₁-C₆ alkyl)C(═O)N(H)(C₁-C₆ alkyl), —N(C₁-C₆ alkyl)C(═O)NH₂, —OC(═O)C₁-C₆ alkyl, —OC(═O)OC₁—C₆ alkyl, —OP(═O)(OC₁—C₆ alkyl)₂, —SC(═O)OC₁—C₆ alkyl and —SC(═O)N(C₁-C₆ alkyl)₂, wherein any alkyl portion of R^{f} is optionally substituted with halogen; and

R^{e} is selected from the group consisting of halogen, OH, C₁-C₆ alkyl and oxo; and R^{g} is selected from the group consisting of C₁-C₆ alkyl and C₃-C₆ cycloalkyl, wherein R^{g} may be optionally substituted, such as by halogen or oxo.

In some embodiments, a compound of Formula (0) is further defined as a compound of Formula (0a):

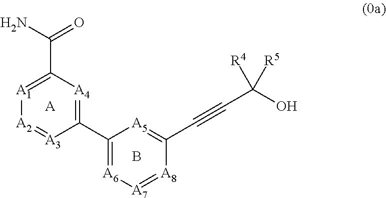

or a stereoisomer or salt thereof, wherein:

ring A is a monocycle or a fused bicycle;

A₁ is N or CR¹;

A₂ is NR² or CR²;

A₃ is NR³ or CR³;

A₄ is N or CH; and

one, two or three of A₁-A₄ is N, wherein:
R¹ is selected from the group consisting of H, halogen, NR^{a}R^{b}, C₁-C₃ alkyl, C₃-C₇ cycloalkyl, C₁-C₃ alkoxy and 3-11 membered heterocyclyl, wherein each of R¹ is optionally substituted by F, OH, CN, SH, CH₃, or CF₃;
R² is selected from the group consisting of H, NR^{a}R^{b} C₁-C₆ alkyl, C₃-C₇ cycloalkyl, C₁-C₆ alkoxy, 3-6 membered heterocyclyloxy, phenyl and 3-11 membered heterocyclyl, wherein each of R² is optionally substituted by R^{c};
R³ is selected from the group consisting of H, C₁-C₆ alkyl, NR^{a}R^{b} and halogen; or
R¹ and R² taken together form a cyclic group selected from the group consisting of C₃-C₇ cycloalkyl, phenyl and 3-11 membered heterocyclyl, wherein the cyclic group is optionally substituted by R^{d}; or
R² and R³ taken together form a cyclic group selected from the group consisting of C₃-C₇ cycloalkyl, phenyl and a 3-11 membered heterocyclyl, wherein the cyclic group is optionally substituted by R^{d};

R⁴ is selected from the group consisting of C₁-C₆ alkyl, CH₂F and CH₂OH;

R⁵ is 3-11 membered heterocyclyl optionally substituted by R^{e}; or

R⁴ and R⁵ together form a C₃-C₁₁ cycloalkyl optionally substituted by R^{e} or a 3-11 membered heterocyclyl optionally substituted by R^{e};

one of A₅-A₈ is N and the remaining are CR⁶ or all are CR⁶;

R⁶, independently at each occurrence, is selected from the group consisting of H, F, Cl, NH₂, NHCH₃, N(CH₃)₂, OH, OCH₃, OCHF₂, OCH₂F, OCF₃, SH, SCH₃, SCHF₂, SCH₂F, CN, CH₃, CHF₂, CH₂F, CH₂OH, CF₃, NO₂ and N₃; or

two R⁶ are taken together to form a 5-6 membered heterocyclyl optionally substituted by R^{e};

R^{a} is selected from the group consisting of H and C₁-C₆ alkyl optionally substituted by C₁-C₃ alkoxy, F, OH, CN, SH, CH₃ or CF₃;

R^{b} is selected from the group consisting of H, C₁-C₆ alkyl, C₁-C₆ alkoxy, C₃-C₆ cycloalkyl, C(O)R^{g}, phenyl and 3-11 membered heterocyclyl wherein R^{b} may be optionally substituted by C₁-C₃ alkoxy, F, OH, CN, SH, CH₃, CF₃, or 3-6 membered heterocyclyl optionally substituted by R^{e};

R^{c} and R^{d} are each independently selected from the group consisting of halogen, —(X¹)₀₋₁—CN, —(X¹)₀₋₁—NO₂, —(X¹)₀₋₁—SF₅, —(X¹)₀₋₁—OH, —(X¹)₀₋₁—NH₂, —(X¹)₀₋₁—N(H)(R^{1a}), —(X¹)₀₋₁—N(R^{1b})(R^{1a}), —(X¹)₀₋₁—CF₃, C₁-C₆ alkyl, C₁-C₆ haloalkyl, C₁-C₆ heteroalkyl, C₁-C₆ alkoxy, C₁-C₆ alkylthio, oxo, —(X¹)₀₋₁—C₁-C₆ alkyl, —(X¹)₀₋₁—C₃-C₁₀ cycloalkyl, —(X¹)₀₋₁-3-11 membered heterocyclyl, —(X¹)₀₋₁—C₆-C₁₀ aryl, —C(═O)(X¹)₁—C₃-C₁₀ cycloalkyl, —C(═O)(X¹)₁-3-11 membered heterocyclyl, —(X¹)₀₋₁—C(═Y¹)N(H)(R^{1a}), —(X¹)₀₋₁—C(═Y¹)NH₂, —(X¹)₀₋₁—C(═Y¹)N(R^{1a})(R^{1b}), —(X¹)₀₋₁—C(═Y¹)OR^{1a}, —(X¹)₀₋₁—C(═Y¹)OH, —(X¹)₀₋₁—N(H)C(═Y¹)(R^{1a}), —(X¹)₀₋₁—N(R^{1b})C(═Y¹)(R^{1a}), —(X¹)₀₋₁—N(R^{1b})C(═Y¹)(H), —(X¹)₀₋₁—N(H)C(═Y¹)OR^{1a}, —(X¹)₀₋₁—N(R^{1b})C(═Y¹)OR^{1a}, —(X¹)₀₋₁—S(O)₁₋₂R^{1a}, —(X¹)₀₋₁—N(H)S(O)₁₋₂R^{1a}, —(X¹)₀₋₁—N(R^{1b})S(O)₁₋₂R^{1a}, —(X¹)₀₋₁—S(O)₀₋₁N(H)(R^{1a}), —(X¹)₀₋₁—S(O)₀₋₁N(R^{1b})(R^{1a}), —(X¹)₀₋₁—S(O)₀₋₁NH₂, —(X¹)₀₋₁—S(═O)(═NR^{1b})R^{1a}, —(X¹)₀₋₁—C(═Y¹)R^{1a}, —(X¹)₀₋₁—C(═Y¹)H, —(X¹)₀₋₁—C(═NOH)R^{1a}, —(X¹)₀₋₁—C(═NOR^{1b})R^{1a}, —(X¹)₀₋₁—NHC(═Y¹)N(H)(R^{1a}), —(X¹)₀₋₁—NHC(═Y¹)NH₂, —(X¹)₀₋₁—NHC(═Y¹)N(R^{1b})(R^{1a}), —(X¹)₀₋₁—N(R^{1a})C(═Y¹)N(H)(R^{1a}), —(X¹)₀₋₁—N(R^{1a})C(═Y¹)N(R^{1a})(R^{1b}), —(X¹)₀₋₁—N(R^{1a})C(═Y¹)NH₂, —(X¹)₀₋₁—OC(═Y¹)R^{1a}, —(X¹)₀₋₁—OC(═Y¹)H, —(X¹)₀₋₁—OC(═Y¹)OR^{1a}, —(X¹)₀₋₁—OP(═Y¹)(OR^{1a})(OR^{1b}), —(X¹)—SC(═Y¹)OR^{1a} and —(X¹)—SC(═Y¹)N(R^{1a})(R^{1b}) wherein X¹ is selected from the group consisting of C₁-C₆ alkylene, C₁-C₆ heteroalkylene, C₂-C₆ alkenylene, C₂-C₆ alkynylene, C₁-C₆ alkyleneoxy, C₃-C₇ cycloalkylene, 3-11 membered heterocyclylene and phenylene; R^{1a} and R^{1b} are each independently selected from the group consisting of C₁-C₆ alkyl, C₁-C₆ haloalkyl, C₁-C₆ heteroalkyl, C₃-C₇ cycloalkyl, (C₃-C₇ cycloalkylene)C₁-C₆ alkyl, 3-11 membered heterocyclyl, (3-11 membered heterocyclylene)C₁-C₆ alkyl, C₆ aryl, and (C₆-C₁₀ arylene)C₁-C₆ alkyl, or R^{1a} and R^{1b} when attached to the same nitrogen atom are optionally combined to form a 3-11 membered heterocyclyl comprising 0-3 additional heteroatoms selected from N, O and S; Y¹ is O, NR^{1c} or S wherein R^{1c} is H or C₁-C₆ alkyl; wherein any portion of an R^{c} or R^{d} substituent, including R^{1a}, R^{1b} and R^{1c}, at each occurrence is each independently further substituted by from 0 to 4 R^{f} substituents selected from the group consisting of halogen, CN, NO₂, SF₅, OH, NH₂, —N(C₁-C₆ alkyl)₂, —NH(C₁-C₆ alkyl), oxo, C₁-C₆ alkyl, C₁-C₆ hydroxyalkyl, C₁-C₆ heteroalkyl, C₁-C₆ alkoxy, C₁-C₆ alkylthio, C₃-C₇ cycloalkyl, 3-11 membered heterocyclyl, —C(═O)N(H)(C₁-C₆ alkyl), —C(═O)N(C₁-C₆ alkyl)₂, —C(═O)NH₂, —C(═O)OC₁—C₆ alkyl, —C(═O)OH, —N(H)C(═O)(C₁-C₆ alkyl), —N(C₁-C₆ alkyl)C(═O)(C₁-C₆ alkyl), —N(H)C(═O)OC₁—C₆ alkyl, —N(C₁-C₆ alkyl)C(═O)OC₁—C₆ alkyl, —S(O)₁₋₂C₁-C₆ alkyl, —N(H)S(O)₁₋₂C₁-C₆ alkyl, —N(C₁-C₆ alkyl)S(O)₁₋₂C₁-C₆ alkyl, —S(O)₀₋₁N(H)(C₁-C₆ alkyl), —S(O)₀₋₁N(C₁-C₆ alkyl)₂, —S(O)₀₋₁NH₂, —C(═O)C₁-C₆ alkyl, —C(═O)C₃-C₇ cycloalkyl, —C(═NOH)C₁-C₆ alkyl, —C(═NOC₁—C₆ alkyl)C₁-C₆ alkyl, —NHC(═O)N(H)(C₁-C₆ alkyl), —NHC(═O)N(C₁-C₆ alkyl)₂, —NHC(═O)NH₂, —N(C₁-C₆ alkyl)C(═O)N(H)(C₁-C₆ alkyl), —N(C₁-C₆ alkyl)C(═O)NH₂, —OC(═O)C₁-C₆ alkyl, —OC(═O)OC₁—C₆ alkyl, —OP(═O)(OC₁—C₆ alkyl)₂, —SC(═O)OC₁—C₆ alkyl and —SC(═O)N(C₁-C₆ alkyl)₂, wherein any alkyl portion of R^{f} is optionally substituted with halogen; and

R^{e} is selected from the group consisting of halogen, OH, C₁-C₆ alkyl and oxo; and R^{g} is selected from the group consisting of C₁-C₆ alkyl and C₃-C₆ cycloalkyl, wherein R^{g} may be optionally substituted, such as by halogen or oxo.

In some embodiments, a compound of Formula (0) is further defined as a compound of Formula (0-0):

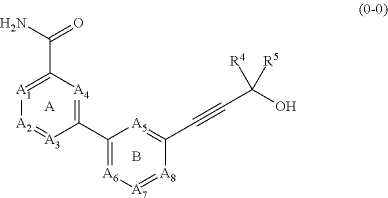

or a stereoisomer or salt thereof, wherein:

ring A is a monocycle or a fused bicycle;

A₁ is NR¹ or CR¹;

A₂ is NR² or CR²;

A₃ is N or CR³;

A₄ is N; and

one, two or three of A₁-A₄ is N, wherein:
R¹ is selected from the group consisting of H, halogen, NR^{a}R^{b}, C₁-C₃ alkyl, C₃-C₇ cycloalkyl, C₁-C₃ alkoxy and 3-11 membered heterocyclyl, wherein each of R¹ is optionally substituted by F, OH, CN, SH, CH₃ or CF₃;
R² is selected from the group consisting of H, NR^{a}R^{b} C₁-C₆ alkyl, C₃-C₇ cycloalkyl, C₁-C₆ alkoxy, phenyl and 3-11 membered heterocyclyl, wherein each of R² is optionally substituted by R^{e};
R³ is selected from the group consisting of H and halogen; or
R¹ and R² taken together form a cyclic group selected from the group consisting of C₃-C₇ cycloalkyl, phenyl and 3-11 membered heterocyclyl, wherein the cyclic group is optionally substituted by R^{d}; or
R² and R³ taken together form a cyclic group selected from the group consisting of C₃-C₇ cycloalkyl, phenyl and a 3-11 membered heterocyclyl, wherein the cyclic group is optionally substituted by R^{d};

R⁴ is selected from the group consisting of C₁-C₆ alkyl, CH₂F and CH₂OH;

R⁵ is 3-11 membered heterocyclyl optionally substituted by R^{e}; or

R⁴ and R⁵ together form a C₃-C₁₁ cycloalkyl optionally substituted by R^{e} or a 3-11 membered heterocyclyl optionally substituted by R^{e};

one of A₅-A₈ is N and the remaining are CR⁶ or all are CR⁶;

R⁶, independently at each occurrence, is selected from the group consisting of H, F, Cl, NH₂, NHCH₃, N(CH₃)₂, OH, OCH₃, OCHF₂, OCH₂F, OCF₃, SH, SCH₃, SCHF₂, SCH₂F, CN, CH₃, CHF₂, CH₂F, CH₂OH, CF₃, NO₂ and N₃;

R^{a} is selected from the group consisting of H and C₁-C₆ alkyl optionally substituted by C₁-C₃ alkoxy, F, OH, CN, SH, CH₃ or CF₃;

R^{b} is selected from the group consisting of H, C₁-C₆ alkyl, C₁-C₆ alkoxy, C₃-C₆ cycloalkyl, C(O)R^{g}, phenyl and 3-11 membered heterocyclyl wherein R^{b} may be optionally substituted by C₁-C₃ alkoxy, F, OH, CN, SH, CH₃ or CF₃;

R^{c} and R^{d} are each independently selected from the group consisting of halogen, —(X¹)₀₋₁—CN, —(X¹)₀₋₁—NO₂, —(X¹)₀₋₁—SF₅, —(X¹)₀₋₁—OH, —(X¹)₀₋₁—NH₂, —(X¹)₀₋₁—N(H)(R^{1a}), —(X¹)₀₋₁—N(R^{1b})(R^{1a}), —(X¹)₀₋₁—CF₃, C₁-C₆ alkyl, C₁-C₆ haloalkyl, C₁-C₆ heteroalkyl, C₁-C₆ alkoxy, C₁-C₆ alkylthio, oxo, —(X¹)₀₋₁—C₁-C₆ alkyl, —(X¹)₀₋₁—C₃-C₁₀ cycloalkyl, —(X¹)₀₋₁-3-11 membered heterocyclyl, —(X¹)₀₋₁—C₆-C₁₀ aryl, —C(═O)(X¹)₁—C₃-C₁₀ cycloalkyl, —C(═O)(X¹)₁-3-11 membered heterocyclyl, —(X¹)₀₋₁—C(═Y¹)N(H)(R^{1a}), —(X¹)₀₋₁—C(═Y¹)NH₂, —(X¹)₀₋₁—C(═Y¹)N(R^{1a})(R^{1b}), —(X¹)₀₋₁—C(═Y¹)OR^{1a}, —(X¹)₀₋₁—C(═Y¹)OH, —(X¹)₀₋₁—N(H)C(═Y¹)(R^{1a}), —(X¹)₀₋₁—N(R^{1b})C(═Y¹)(R^{1a}), —(X¹)₀₋₁—N(R^{1b})C(═Y¹)(H), —(X¹)₀₋₁—N(H)C(═Y¹)OR^{1a}, —(X¹)₀₋₁—N(R^{1b})C(═Y¹)OR^{1a}, —(X¹)₀₋₁—S(O)₁₋₂R^{1a}, —(X¹)₀₋₁—N(H)S(O)₁₋₂R^{1a}, —(X¹)₀₋₁—N(R^{1b})S(O)₁₋₂R^{1a}, —(X¹)₀₋₁—S(O)₀₋₁N(H)(R^{1a}), —(X¹)₀₋₁—S(O)₀₋₁N(R^{1b})(R^{1a}), —(X¹)₀₋₁—S(O)₀₋₁NH₂, —(X¹)₀₋₁—S(═O)(═NR^{1b})R^{1a}, —(X¹)₀₋₁—C(═Y¹)R^{1a}, —(X¹)₀₋₁—C(═Y¹)H, —(X¹)₀₋₁—C(═NOH)R^{1a}, —(X¹)₀₋₁—C(═NOR^{1b})R^{1a}, —(X¹)₀₋₁—NHC(═Y¹)N(H)(R^{1a}), —(X¹)₀₋₁—NHC(═Y¹)NH₂, —(X¹)₀₋₁—NHC(═Y¹)N(R^{1b})(R^{1a}), —(X¹)₀₋₁—N(R^{1a})C(═Y¹)N(H)(R^{1a}), —(X¹)₀₋₁—N(R^{1a})C(═Y¹)N(R^{1a})(R^{1b}), —(X¹)₀₋₁—N(R^{1a})C(═Y¹)NH₂, —(X¹)₀₋₁—OC(═Y¹)R^{1a}, —(X¹)₀₋₁—OC(═Y¹)H, —(X¹)₀₋₁—OC(═Y¹)OR^{1a}, —(X¹)₀₋₁—OP(═Y¹)(OR^{1a})(OR^{1b}), —(X¹)—SC(═Y¹)OR^{1a} and —(X¹)—SC(═Y¹)N(R^{1a})(R^{1b}) wherein X¹ is selected from the group consisting of C₁-C₆ alkylene, C₁-C₆ heteroalkylene, C₂-C₆ alkenylene, C₂-C₆ alkynylene, C₁-C₆ alkyleneoxy, C₃-C₇ cycloalkylene, 3-11 membered heterocyclylene and phenylene; R^{1a} and R^{1b} are each independently selected from the group consisting of C₁-C₆ alkyl, C₁-C₆ haloalkyl, C₁-C₆ heteroalkyl, C₃-C₇ cycloalkyl, (C₃-C₇ cycloalkylene)C₁-C₆ alkyl, 3-11 membered heterocyclyl, (3-11 membered heterocyclylene)C₁-C₆ alkyl, C₆ aryl, and (C₆-C₁₀ arylene)C₁-C₆ alkyl, or R^{1a} and R^{1b} when attached to the same nitrogen atom are optionally combined to form a 3-11 membered heterocyclyl comprising 0-3 additional heteroatoms selected from N, O and S; Y¹ is O, NR^{1c} or S wherein R^{1c} is H or C₁-C₆ alkyl; wherein any portion of an R^{c} or R^{d} substituent, including R^{1a}, R^{1b} and R^{1c}, at each occurrence is each independently further substituted by from 0 to 4 R^{f} substituents selected from the group consisting of halogen, CN, NO₂, SF₅, OH, NH₂, —N(C₁-C₆ alkyl)₂, —NH(C₁-C₆ alkyl), oxo, C₁-C₆ alkyl, C₁-C₆ hydroxyalkyl, C₁-C₆ heteroalkyl, C₁-C₆ alkoxy, C₁-C₆ alkylthio, C₃-C₇ cycloalkyl, 3-11 membered heterocyclyl, —C(═O)N(H)(C₁-C₆ alkyl), —C(═O)N(C₁-C₆ alkyl)₂, —C(═O)NH₂, —C(═O)OC₁—C₆ alkyl, —C(═O)OH, —N(H)C(═O)(C₁-C₆ alkyl), —N(C₁-C₆ alkyl)C(═O)(C₁-C₆ alkyl), —N(H)C(═O)OC₁—C₆ alkyl, —N(C₁-C₆ alkyl)C(═O)OC₁—C₆ alkyl, —S(O)₁₋₂C₁-C₆ alkyl, —N(H)S(O)₁₋₂C₁-C₆ alkyl, —N(C₁-C₆ alkyl)S(O)₁₋₂C₁-C₆ alkyl, —S(O)₀₋₁N(H)(C₁-C₆ alkyl), —S(O)₀₋₁N(C₁-C₆ alkyl)₂, —S(O)₀₋₁NH₂, —C(═O)C₁-C₆ alkyl, —C(═O)C₃-C₇ cycloalkyl, —C(═NOH)C₁-C₆ alkyl, —C(═NOC₁—C₆ alkyl)C₁-C₆ alkyl, —NHC(═O)N(H)(C₁-C₆ alkyl), —NHC(═O)N(C₁-C₆ alkyl)₂, —NHC(═O)NH₂, —N(C₁-C₆ alkyl)C(═O)N(H)(C₁-C₆ alkyl), —N(C₁-C₆ alkyl)C(═O)NH₂, —OC(═O)C₁-C₆ alkyl, —OC(═O)OC₁—C₆ alkyl, —OP(═O)(OC₁—C₆ alkyl)₂, —SC(═O)OC₁—C₆ alkyl and —SC(═O)N(C₁-C₆ alkyl)₂, wherein any alkyl portion of R^{f} is optionally substituted with halogen; and

R^{e} is selected from the group consisting of halogen, OH, C₁-C₆ alkyl and oxo; and R^{g} is selected from the group consisting of C₁-C₆ alkyl and C₃-C₆ cycloalkyl, wherein R^{g} may be optionally substituted, such as by halogen or oxo.

In some embodiments, a compound of Formula (0) is further defined as a compound of Formula (I):

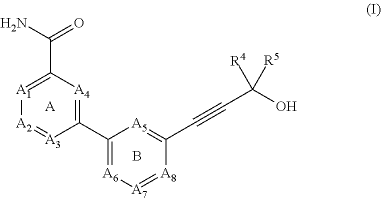

or a stereoisomer or salt thereof, wherein:

ring A is a monocycle or a fused bicycle;

A₁ is N or CR¹;

A₂ is N or CR²;

A₃ is N or CR³;

A₄ is N; and

one, two or three of A₁-A₄ is N, wherein:
R¹ is selected from the group consisting of H, halogen, NR^{a}R^{b}, C₁-C₃ alkyl, C₃-C₇ cycloalkyl, C₁-C₃ alkoxy and 3-11 membered heterocyclyl (e.g., a 4-7 membered heterocycloalkyl or a 5-6 membered heteroaryl), wherein each of R¹ is optionally substituted by F, OH, CN, SH, CH₃ or CF₃;
R² is selected from the group consisting of H, NR^{a}R^{b} C₁-C₆ alkyl, C₃-C₇ cycloalkyl, C₁-C₆ alkoxy, phenyl and 3-11 membered heterocyclyl (e.g., a 4-7 membered heterocycloalkyl or a 5-6 membered heteroaryl), wherein each of R² is optionally substituted by R^{c};
R³ is selected from the group consisting of H and halogen; or
R¹ and R² taken together form a cyclic group selected from the group consisting of C₃-C₇ cycloalkyl, phenyl and 3-11 membered heterocyclyl (e.g., a 4-7 membered heterocycloalkyl or a 5-6 membered heteroaryl), wherein the cyclic group is optionally substituted by R^{d}; or
R² and R³ taken together form a cyclic group selected from the group consisting of C₃-C₇ cycloalkyl, phenyl and a 3-11 membered heterocyclyl (e.g., a 4-7 membered heterocycloalkyl or a 5-6 membered heteroaryl), wherein the cyclic group is optionally substituted by R^{d};

R⁴ is selected from the group consisting of C₁-C₆ alkyl, CH₂F and CH₂OH;

R⁵ is 3-11 membered heterocyclyl (e.g., a 4-7 membered heterocycloalkyl or a 5-6 membered heteroaryl) optionally substituted by R^{e}; or

R⁴ and R⁵ together form a C₃-C₁₁ cycloalkyl optionally substituted by R^{e} or a 3-11 membered heterocyclyl (e.g., a 4-7 membered heterocycloalkyl or a 5-6 membered heteroaryl) optionally substituted by R^{e};

one of A₅-A₈ is N and the remaining are CR⁶ or all are CR⁶;

R⁶, independently at each occurrence, is selected from the group consisting of H, F, Cl, NH₂, NHCH₃, N(CH₃)₂, OH, OCH₃, OCHF₂, OCH₂F, OCF₃, SH, SCH₃, SCHF₂, SCH₂F, CN, CH₃, CHF₂, CH₂F, CH₂OH, CF₃, NO₂ and N₃;

R^{a} is selected from the group consisting of H and C₁-C₆ alkyl optionally substituted by C₁-C₃ alkoxy, F, OH, CN, SH, CH₃ or CF₃;

R^{b} is selected from the group consisting of H, C₁-C₆ alkyl, C₁-C₆ alkoxy, C₃-C₆ cycloalkyl, C(O)R^{g}, phenyl and 3-11 membered heterocyclyl (e.g., a 4-7 membered heterocycloalkyl or a 5-6 membered heteroaryl) wherein R^{b} may be optionally substituted by C₁-C₃ alkoxy, F, OH, CN, SH, CH₃ or CF₃;

R^{c} and R^{d} are each independently selected from the group consisting of halogen, —(X¹)₀₋₁—CN, —(X¹)₀₋₁—NO₂, —(X¹)₀₋₁—SF₅, —(X¹)₀₋₁—OH, —(X¹)₀₋₁—NH₂, —(X¹)₀₋₁—N(H)(R^{1a}), —(X¹)₀₋₁—N(R^{1b})(R^{1a}),)CF₃, C₁-C₆ alkyl, C₁-C₆ haloalkyl, C₁-C₆ heteroalkyl, C₁-C₆ alkoxy, C₁-C₆ alkylthio, oxo, —(X¹)₀₋₁—C₁-C₆ alkyl, —(X¹)₀₋₁—C₃-C₁₀ cycloalkyl, —(X¹)₀₋₁-3-11 membered heterocyclyl (e.g., a 4-7 membered heterocycloalkyl or a 5-6 membered heteroaryl), —(X¹)₀₋₁—C₆-C₁₀ aryl, —C(═O)(X¹)₁—C₃-C₁₀ cycloalkyl, —C(═O)(X¹)₁-3-11 membered heterocyclyl, —(X¹)₀₋₁—C(═Y¹)N(H)(R^{1a}), —(X¹)₀₋₁—C(═Y¹)NH₂, —(X¹)₀₋₁—C(═Y¹)N(R^{1a})(R^{1b}), —(X¹)₀₋₁—C(═Y¹)OR^{1a}, —(X¹)₀₋₁—C(═Y¹)OH, —(X¹)₀₋₁—N(H)C(═Y¹)(R^{1a}), —(X¹)₀₋₁—N(R^{1b})C(═Y¹)(R^{1a}), —(X¹)₀₋₁—N(R^{1b})C(═Y¹)(H), —(X¹)₀₋₁—N(H)C(═Y¹)OR^{1a}, —(X¹)₀₋₁—N(R^{1b})C(═Y¹)OR^{1a}, —(X¹)₀₋₁—S(O)₁₋₂R^{1a}, —(X¹)₀₋₁—N(H)S(O)₁₋₂R^{1a}, —(X¹)₀₋₁—N(R^{1b})S(O)₁₋₂R^{1a}, —(X¹)₀₋₁—S(O)₀₋₁N(H)(R^{1a}), —(X¹)₀₋₁—S(O)₀₋₁N(R^{1b})(R^{1a}), —(X¹)₀₋₁—S(O)₀₋₁NH₂, —(X¹)₀₋₁—S(═O)(═NR^{1b})R^{1a}, —(X¹)₀₋₁—C(═Y¹)R^{1a}, —(X¹)₀₋₁—C(═Y¹)H, —(X¹)₀₋₁—C(═NOH)R^{1a}, —(X¹)₀₋₁—C(═NOR^{1b})R^{1a}, —(X¹)₀₋₁—NHC(═Y¹)N(H)(R^{1a}), —(X¹)₀₋₁—NHC(═Y¹)NH₂, —(X¹)₀₋₁—NHC(═Y¹)N(R^{1b})(R^{1a}), —(X¹)₀₋₁—N(R^{1a})C(═Y¹)N(H)(R^{1a}), —(X¹)₀₋₁—N(R^{1a})C(═Y¹)N(R^{1a})(R^{1b}), —(X¹)₀₋₁—N(R^{1a})C(═Y¹)NH₂, —(X¹)₀₋₁—OC(═Y¹)R^{1a}, —(X¹)₀₋₁—OC(═Y¹)H, —(X¹)₀₋₁—OC(═Y¹)OR^{1a}, —(X¹)₀₋₁—OP(═Y¹)(OR^{1a})(OR^{1b}), —(X¹)—SC(═Y¹)OR^{1a} and —(X¹)—SC(═Y¹)N(R^{1a})(R^{1b}) wherein X¹ is selected from the group consisting of C₁-C₆ alkylene, C₁-C₆ heteroalkylene, C₂-C₆ alkenylene, C₂-C₆ alkynylene, C₁-C₆ alkyleneoxy, C₃-C₇ cycloalkylene, 3-11 membered heterocyclylene and phenylene; and R^{1b} are each independently selected from the group consisting of C₁-C₆ alkyl, C₁-C₆ haloalkyl, C₁-C₆ heteroalkyl, C₃-C₇ cycloalkyl, (C₃-C₇ cycloalkylene)C₁-C₆ alkyl, 3-11 membered heterocyclyl, (3-11 membered heterocyclylene)C₁-C₆ alkyl, C₆ aryl, and (C₆-C₁₀ arylene)C₁-C₆ alkyl, or R^{1a} and R^{1b} when attached to the same nitrogen atom are optionally combined to form a 3-11 membered heterocyclyl (e.g., a 4-7 membered heterocycloalkyl or a 5-6 membered heteroaryl) comprising 0-3 additional heteroatoms selected from N, O and S; Y¹ is O, NR^{1c} or S wherein R^{1c} is H or C₁-C₆ alkyl; wherein any portion of an R^{c} or R^{d} substituent, including R^{1a}, R^{1b} and R^{1c}, at each occurrence is each independently further substituted by from 0 to 4 R^{f} substituents selected from the group consisting of halogen, CN, NO₂, SF₅, OH, NH₂, —N(C₁-C₆ alkyl)₂, —NH(C₁-C₆ alkyl), oxo, C₁-C₆ alkyl, C₁-C₆ haloalkyl, C₁-C₆ hydroxyalkyl, C₁-C₆ heteroalkyl, C₁-C₆ alkoxy, C₁-C₆ alkylthio, C₃-C₇ cycloalkyl, 3-11 membered heterocyclyl (e.g., a 4-7 membered heterocycloalkyl or a 5-6 membered heteroaryl), —C(═O)N(H)(C₁-C₆ (halo)alkyl), —C(═O)N(C₁-C₆ (halo)alkyl)₂, —C(═O)NH₂, —C(═O)OC₁—C₆ (halo)alkyl, —C(═O)OH, —N(H)C(═O)(C₁-C₆ (halo)alkyl), —N(C₁-C₆ (halo)alkyl)C(═O)(C₁-C₆ (halo)alkyl), —N(H)C(═O)OC₁—C₆ (halo)alkyl, —N(C₁-C₆ (halo)alkyl)C(═O)OC₁—C₆ (halo)alkyl, —S(O)₁₋₂C₁-C₆ (halo)alkyl, —N(H)S(O)₁₋₂C₁-C₆ (halo)alkyl, —N(C₁-C₆ (halo)alkyl)S(O)₁₋₂C₁-C₆ (halo)alkyl, —S(O)₀₋₁N(H)(C₁-C₆ (halo)alkyl), —S(O)₀₋₁N(C₁-C₆ (halo)alkyl)₂, —S(O)₀₋₁NH₂, —C(═O)C₁-C₆ (halo)alkyl, —C(═O)C₃-C₇ cycloalkyl, —C(═NOH)C₁-C₆ (halo)alkyl, —C(═NOC₁—C₆ alkyl)C₁-C₆ (halo)alkyl, —NHC(═O)N(H)(C₁-C₆ (halo)alkyl), —NHC(═O)N(C₁-C₆ (halo)alkyl)₂, —NHC(═O)NH₂, —N(C₁-C₆ (halo)alkyl)C(═O)N(H)(C₁-C₆ (halo)alkyl), —N(C₁-C₆ (halo)alkyl)C(═O)NH₂, —OC(═O)C₁-C₆ (halo)alkyl, —OC(═O)OC₁—C₆ (halo)alkyl, —OP(═O)(OC₁—C₆ (halo)alkyl)₂, —SC(═O)OC₁—C₆ (halo)alkyl and —SC(═O)N(C₁-C₆ (halo)alkyl)₂;

R^{e} is selected from the group consisting of halogen, OH, C₁-C₆ alkyl optionally substituted by halogen, and oxo; and

R^{g} is selected from the group consisting of C₁-C₆ alkyl and C₃-C₆ cycloalkyl, wherein R^{g} may be optionally substituted, such as by halogen or oxo.

In some embodiments, a compound of Formula (0) is further defined as a compound of Formula (II):

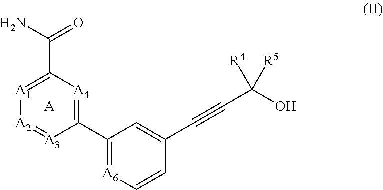

or a stereoisomer or salt thereof, wherein:

ring A is a monocycle or a fused bicycle;

A₁ is N or CR¹;

A₂ is N or CR²;

A₃ is N or CR³;

A₄ is N; and

one or two of A₁-A₄ are N, wherein:
R¹ is is selected from the group consisting of H, NR^{a}R^{b}, C₁-C₃ alkyl, C₁-C₃ alkoxy, and 3-11 membered heterocyclyl (e.g., a 4-7 membered heterocycloalkyl or a 5-6 membered heteroaryl), wherein each of R¹ is optionally substituted by F, OH, CN, SH, CH₃ or CF₃;
R² is selected from the group consisting of H, C₁-C₆ alkyl, C₁-C₆ haloalkyl, C₁-C₆ alkoxy, NR^{a}R^{b}, phenyl and 3-11 membered heterocyclyl (e.g., a 4-7 membered heterocycloalkyl or a 5-6 membered heteroaryl), wherein R² is optionally substituted by R^{c};
R³ is selected from the group consisting of H and halogen; or
R¹ and R² taken together form a cyclic group selected from the group consisting of C₃-C₇ cycloalkyl, phenyl and 3-6 membered heterocyclyl, wherein the cyclic group is optionally substituted by R^{d}; or
R² and R³ taken together form a C₃-C₆ cycloalkyl or a 3-6 membered heterocyclyl, wherein said cycloalkyl and said heterocyclyl are each optionally substituted by R^{d};

R⁴ is C₁-C₃ alkyl;

R⁵ is 3-11 membered heterocyclyl (e.g., a 4-7 membered heterocycloalkyl or a 5-6 membered heteroaryl) optionally substituted by R^{e}; or

R⁴ and R⁵ together form a C₃-C₁₁ cycloalkyl optionally substituted by R^{e} or a 3-11 membered heterocyclyl (e.g., a 4-7 membered heterocycloalkyl or a 5-6 membered heteroaryl) optionally substituted by R^{e};

A₆ is N, CH or CR⁶;

R⁶ is selected from the group consisting of F, Cl, NH₂, NHCH₃, N(CH₃)₂, OH, OCH₃, OCHF₂, OCH₂F, OCF₃, SH, SCH₃, SCHF₂, SCH₂F, CN, CH₃, CHF₂, CH₂F, CF₃ and N₃;

R^{a} is selected from the group consisting of H and C₁-C₆ alkyl and C₁-C₆ haloalkyl;

R^{b} is selected from the group consisting of H, C₁-C₆ alkoxy, C₃-C₆ cycloalkyl, 3-6 membered heterocyclyl, or C₁-C₆ alkyl optionally substituted by C₁-C₆ alkoxy;

R^{c} and R^{d} are each independently selected from the group consisting of halogen, OH, C₁-C₆ alkyl, C₃-C₆ cycloalkyl, C₁-C₆ alkoxy, C₁-C₆ alkylamino, C₁-C₆ dialkylamino, C(O)(C₁-C₆ alkyl), C(O)₂(C₁-C₆ alkyl), phenyl, and 3-6 membered heterocyclyl, wherein each of R^{c} and R^{d} are each independently optionally substituted by halogen, OH, C₁-C₃ haloalkyl, C₁-C₃ alkoxy, 5-6 membered heterocyclyl, or oxo; and

R^{e} is selected from the group consisting of halogen, OH, C₁-C₆ alkyl and oxo.

In some embodiments, R⁴ is CH₃. In some embodiments, R⁵ is a 5-6 membered heterocyclyl optionally substituted by R^{e}.

In some embodiments, such as in a compound of Formula (0), (0a), (0-0), (I), or (II), the following moiety,

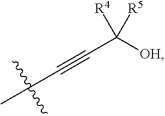

is defined as

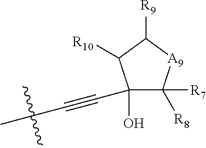

wherein:

A₉ is O, NR¹¹ or CR¹¹R¹², wherein R¹¹ and R¹² are each independently selected from the group consisting of H, halogen, OH and C₁-C₃ alkyl;

R⁷ and R⁸ are each independently selected from halogen, OH and C₁-C₆ alkyl, or R⁷ and R⁸ together form ═O, and

R⁹ and R¹⁰ are each independently selected from R^{e}, or R⁹ and R¹⁰ together form a C₅-C₆ cycloalkyl or a 5-6 membered heterocyclyl, wherein said cycloalkyl and said heterocyclyl are each optionally substituted by R^{e}.

In some embodiments, R⁴ and R⁵ together form a C₈-C₁₀ cycloalkyl optionally substituted by R^{e}. In some embodiments, R⁴ and R⁵ together form a 4-9 membered heterocyclyl optionally substituted by R^{e}.

In some embodiments,

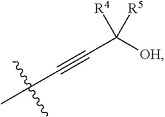

is selected from the group consisting of

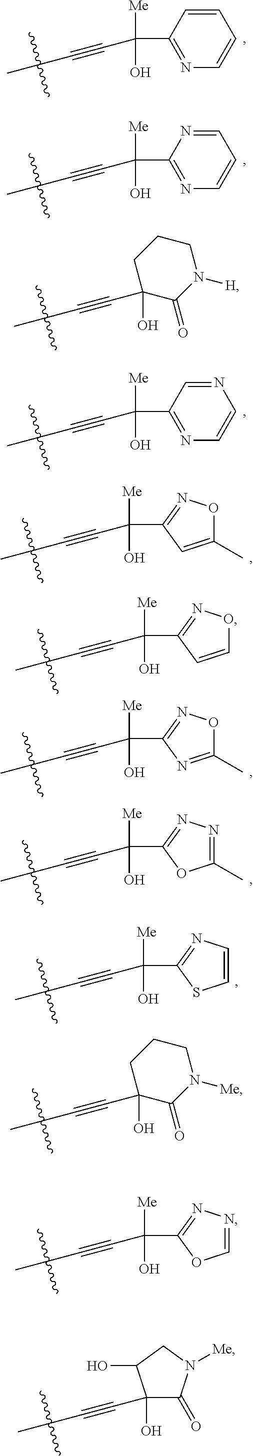

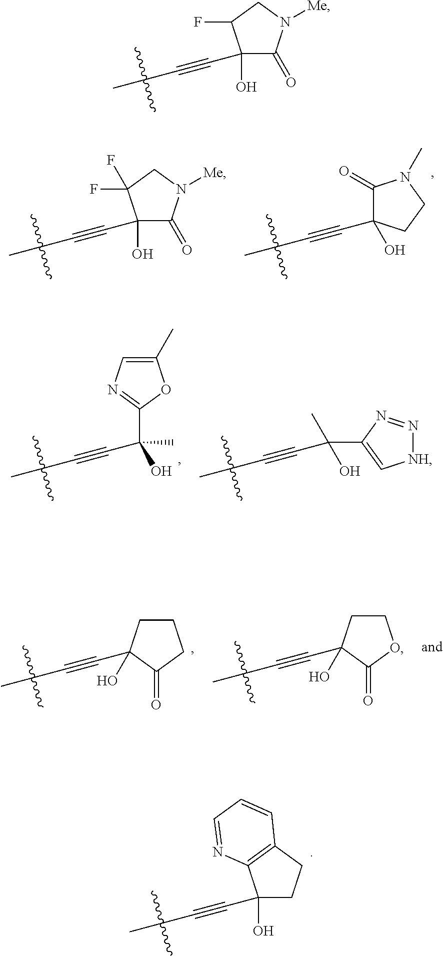

In some embodiments, a compound of Formula (0) is further defined as a compound of Formula (III):

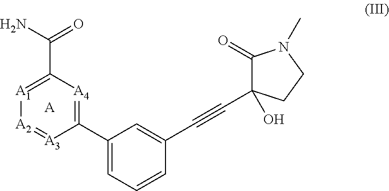

or a stereoisomer or salt thereof, wherein:

ring A is a monocycle or a fused bicycle;

A₁ is N or CR¹;

A₂ is N or CR²;

A₃ is N or CR³;

A₄ is N; and

one or two of A₁-A₄ are N, wherein:
R¹ is H;
R² is selected from the group consisting of H, C₁-C₆ alkyl, C₁-C₆ haloalkyl, C₁-C₆ alkoxy, NR^{a}R^{b} and 3-11 membered heterocyclyl (e.g., a 4-7 membered heterocycloalkyl or a 5-6 membered heteroaryl), wherein R₂ is optionally substituted by R^{c};
R³ is selected from the group consisting of H and halogen; or
R¹ and R² taken together form a cyclic group selected from the group consisting of C₃-C₇ cycloalkyl, phenyl and 3-11 membered heterocyclyl (e.g., a 4-7 membered heterocycloalkyl or a 5-6 membered heteroaryl), wherein the cyclic group is optionally substituted by R^{d}; or
R² and R³ taken together form a C₃-C₆ cycloalkyl or a 3-6 membered heterocyclyl optionally substituted by R^{d};

R^{a} is selected from the group consisting of H and C₁-C₆ alkyl;

R^{b} is selected from the group consisting of H, C₁-C₆ alkoxy, C₃-C₆ cycloalkyl, 3-11 membered heterocyclyl (e.g., a 4-7 membered heterocycloalkyl or a 5-6 membered heteroaryl), or C₁-C₆ alkyl optionally substituted by C₁-C₆ alkoxy;

R^{c} is selected from the group consisting of halogen, OH, C(O)(C₁-C₆ alkyl), 3-11 membered heterocyclyl (e.g., a 4-7 membered heterocycloalkyl or a 5-6 membered heteroaryl), or C₁-C₆ alkyl optionally substituted by C₁-C₆ alkoxy; and

R^{d} is selected from the group consisting of halogen, C₁-C₆ alkyl and C₁-C₆ alkoxy.

In some embodiments, one of A₁-A₄ is N. For example, in some embodiments, A₄ is N. In some embodiments, two of A₁-A₄ is N. For example, in some embodiments, A₁ and A₄ are each N. In other embodiments, A₃ and A₄ are each N.

In some embodiments, R¹ is NR^{a}R^{b} or 3-11 membered heterocyclyl (e.g., a 4-7 membered heterocycloalkyl or a 5-6 membered heteroaryl).

In some embodiments, R² is selected from the group consisting of H, C₁-C₆ alkyl, trifluoromethyl and methoxy. In some embodiments, R² is NR^{a}R^{b}, wherein R^{a} is H or CH₃ and R^{b} is selected from the group consisting of H, CH₃, cyclopropyl, CH₂CH₂OCH₃, OCH₃ and 5-6 membered heterocyclyl. In some embodiments, R² is a 3-11 membered heterocycloalkyl.

In some embodiments, R^{c} is selected from the group consisting of F, OH, CH₃, isobutyl, C(O)CH₃, CH₂OCH₃, tetrahydrofuranyl and thienyl.

In some embodiments, R¹ and R² together form a cyclic group selected from the group consisting of C₃-C₆ cycloalkyl, phenyl and 3-6 membered heterocyclyl, wherein the cyclic group is optionally substituted by F or CH₃.

In some embodiments, R² and R³ together form a 5-6 membered heteroaryl where, in some embodiments, the heteroaryl is optionally substituted by R^{d}.

In some embodiments, ring B is monocyclic. In some embodiments, ring B is phenyl.

In some embodiments, a compound of Formula (0) is further defined as one of Formulas (Ia)-(Id), or a stereoisomer or salt thereof:

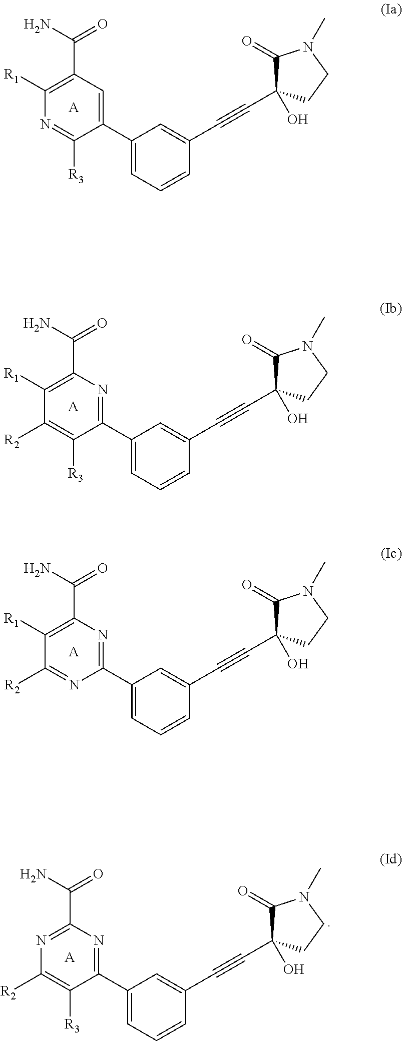

In some embodiments, R^{c} and R^{d} are each independently selected from the group consisting of halogen, —(X¹)₀₋₁—CN, —(X¹)₀₋₁—NO₂, —(X¹)₀₋₁—SF₅, —(X¹)₀₋₁—OH, —(X¹)₀₋₁—NH₂, —(X¹)₀₋₁—N(H)(R^{1a}), —(X¹)₀₋₁—N(R^{1b})(R^{1a})—(X¹)₀₋₁—CF₃, C₁-C₆ alkyl, C₁-C₆ haloalkyl, C₁-C₆ heteroalkyl, C₁-C₆ alkoxy, C₁-C₆ alkylthio, oxo, —(X¹)₀₋₁—C₁-C₆ alkyl, —(X¹)₀₋₁—C₃-C₇ cycloalkyl, —(X¹)₀₋₁-3-11 membered heterocyclyl (e.g., a 4-7 membered heterocycloalkyl or a 5-6 membered heteroaryl), —(X¹)₀₋₁—C₆-C₁₀ aryl, —C(═O)(X¹)₁—C₃-C₇ cycloalkyl, —C(═O)(X¹)₁-3-11 membered heterocyclyl, —(X¹)₀₋₁—C(═Y¹)N(H)(R^{1a}), —(X¹)₀₋₁—C(═Y¹)NH₂, —(X¹)₀₋₁—C(═Y¹)N(R^{1a})(R^{1b}), —(X¹)₀₋₁—C(═Y¹)OR^{1a}, —(X¹)₀₋₁—C(═Y¹)OH, —(X¹)₀₋₁—N(H)C(═Y¹)(R^{1a}), —(X¹)₀₋₁—N(R^{1b})C(═Y¹)(R^{1a}), —(X¹)₀₋₁—N(R^{1b})C(═Y¹)(H), —(X¹)₀₋₁—N(H)C(═Y¹)OR^{1a}, —(X¹)₀₋₁—N(R^{1b})C(═Y¹)OR^{1a}, —(X¹)₀₋₁—S(O)₁₋₂R^{1a}, —(X¹)₀₋₁—N(H)S(O)₁₋₂R^{1a}, —(X¹)₀₋₁—N(R^{1b})S(O)₁₋₂R^{1a}, —(X¹)₀₋₁—S(O)₀₋₁N(H)(R^{1a}), —(X¹)₀₋₁—S(O)₀₋₁N(R^{1b})(R^{1a}), —(X¹)₀₋₁—S(O)₀₋₁NH₂, —(X¹)₀₋₁—S(═O)(═NR^{1b})R^{1a}, —(X¹)₀₋₁—C(═Y¹)R^{1a} and —(X¹)₀₋₁—C(═Y¹)H, wherein X¹ is selected from the group consisting of C₁-C₆ alkylene, C₁-C₆ heteroalkylene, C₂-C₆ alkenylene, C₂-C₆ alkynylene, C₁-C₆ alkyleneoxy, C₃-C₇ cycloalkylene, 3-11 membered heterocyclylene and phenylene; R^{1a} and R^{1b} are each independently selected from the group consisting of C₁-C₆ alkyl, C₁-C₆ haloalkyl, C₁-C₆ heteroalkyl, C₃-C₇ cycloalkyl, 3-11 membered heterocyclyl, and phenyl, or R^{1a} and R^{1b} when attached to the same nitrogen atom are optionally combined to form a 3-11 membered heterocyclyl (e.g., a 4-7 membered heterocycloalkyl or a 5-6 membered heteroaryl) comprising 0-3 additional heteroatoms selected from N, O and S; Y¹ is O, NR^{1c} or S wherein R^{1c} is H or C₁-C₆ alkyl; wherein any portion of an R^{c} or R^{d} substituent, including R^{1a}, R^{1b} and R^{1c}, at each occurrence is each independently further substituted by from 0 to 4 R^{f} substituents selected from the group consisting of halogen, CN, NO₂, OH, NH₂, —N(C₁-C₆ alkyl)₂, —NH(C₁-C₆ alkyl), oxo, C₁-C₆ alkyl, C₁-C₆ haloalkyl, C₁-C₆ hydroxyalkyl, C₁-C₆ heteroalkyl, C₁-C₆ alkoxy, C₁-C₆ alkylthio, C₃-C₇ cycloalkyl, or 3-11 membered heterocyclyl (e.g., a 4-7 membered heterocycloalkyl or a 5-6 membered heteroaryl).

In some embodiments, a heterocyclyl group contains one to three nitrogen atoms, one oxygen atom, or one sulfur atom, or any combination thereof.

In some embodiments, a compound of the present invention is defined as any one or more of the following:

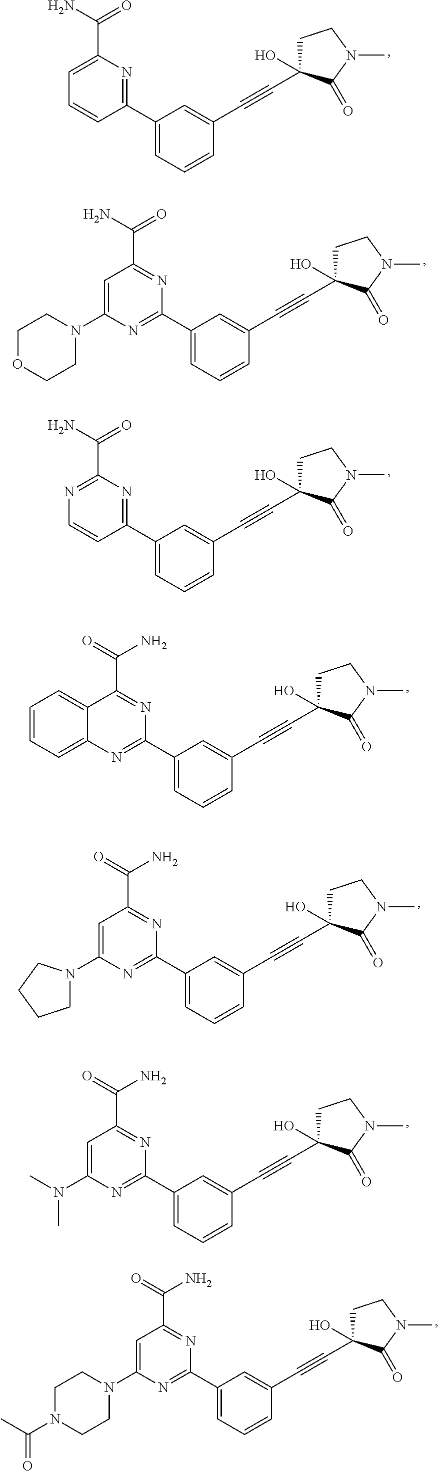

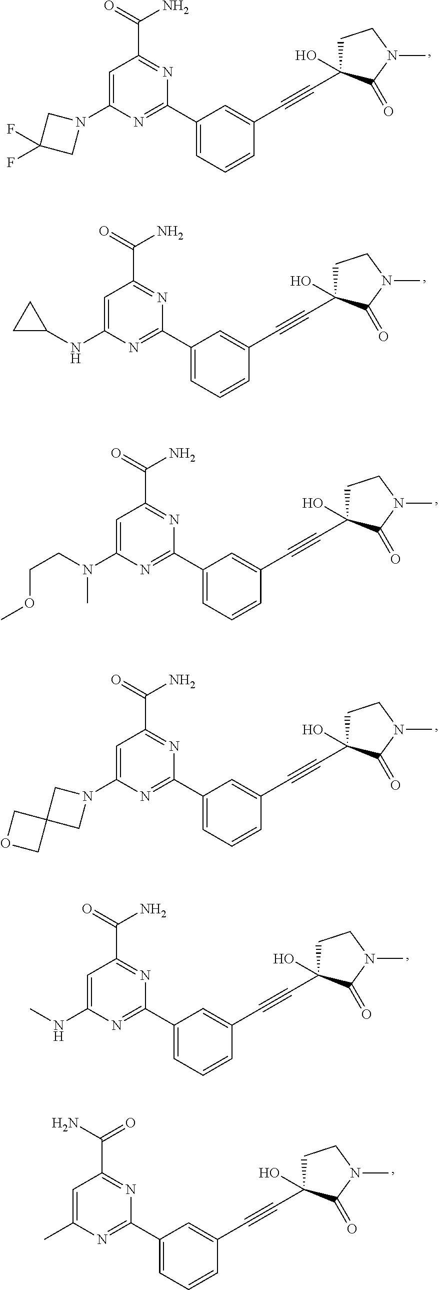

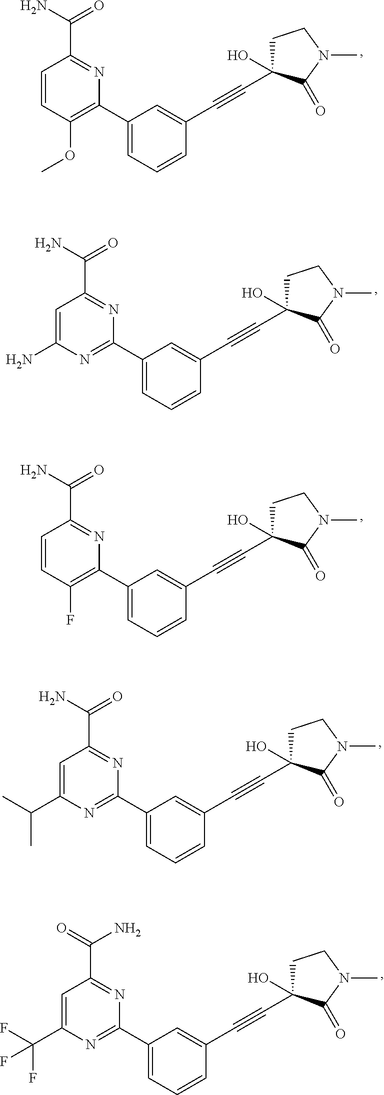

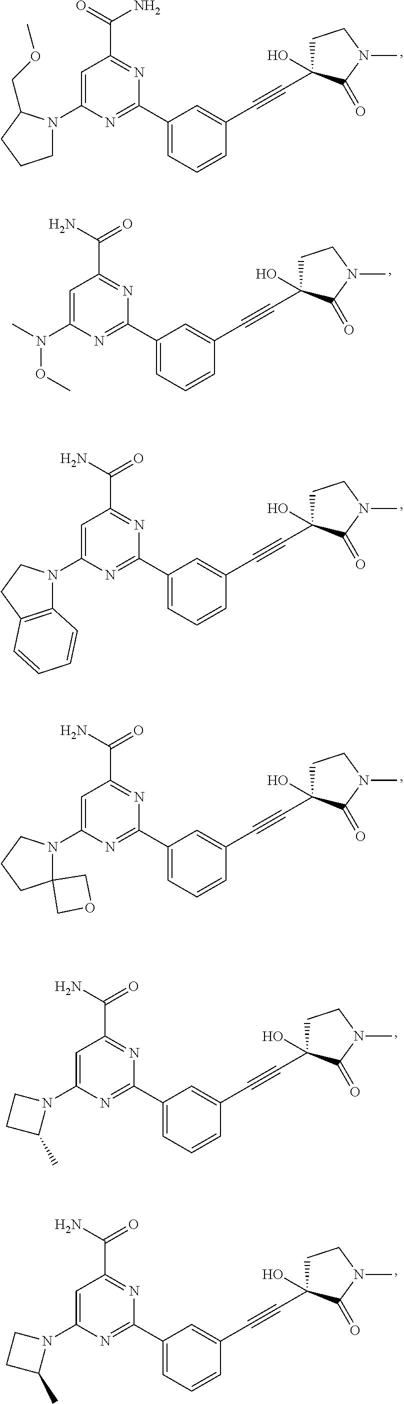

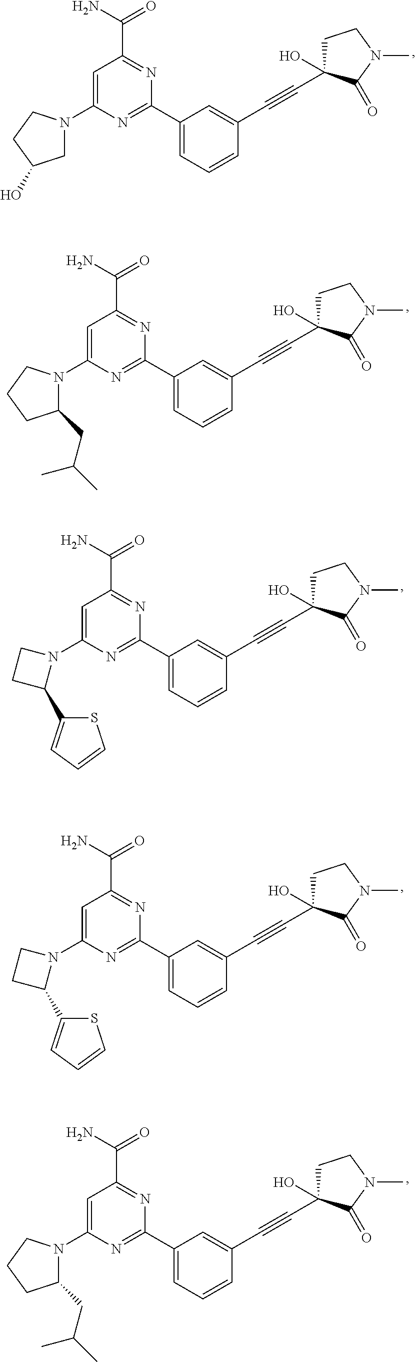 [SMILES: CN1CC[C@@](O)(C#Cc2cccc(-c3nc(C(N)=O)cc(N4CC[C@@H](O)C4)n3)c2)C1=O ; CC(C)C[C@H]1CCCN1c1cc(C(N)=O)nc(-c2cccc(C#C[C@]3(O)CCN(C)C3=O)c2)n1 ; CN1CC[C@@](O)(C#Cc2cccc(-c3nc(C(N)=O)cc(N4CC[C@@H]4c4cccs4)n3)c2)C1=O ; CN1CC[C@@](O)(C#Cc2cccc(-c3nc(C(N)=O)cc(N4CC[C@H]4c4cccs4)n3)c2)C1=O ; CC(C)C[C@@H]1CCCN1c1cc(C(N)=O)nc(-c2cccc(C#C[C@]3(O)CCN(C)C3=O)c2)n1]

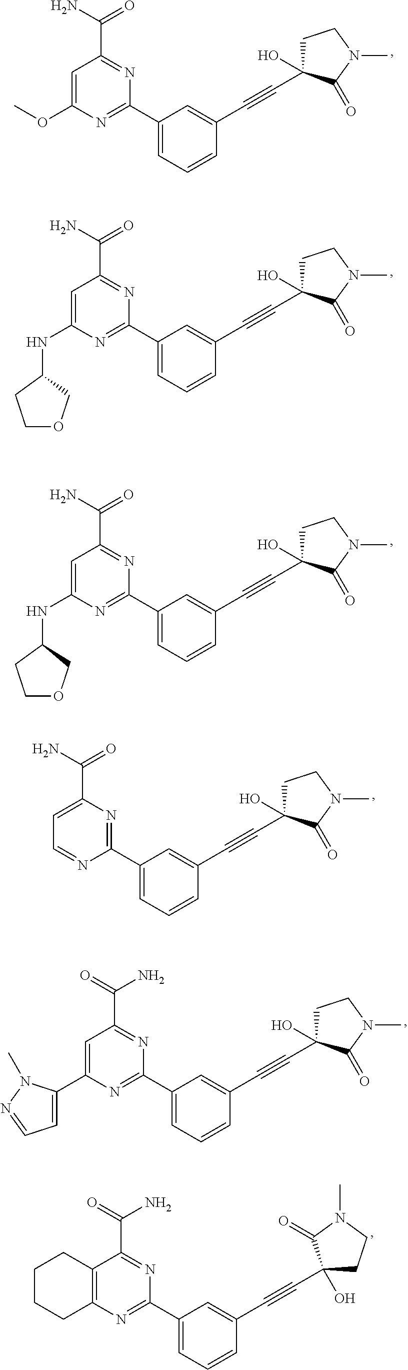

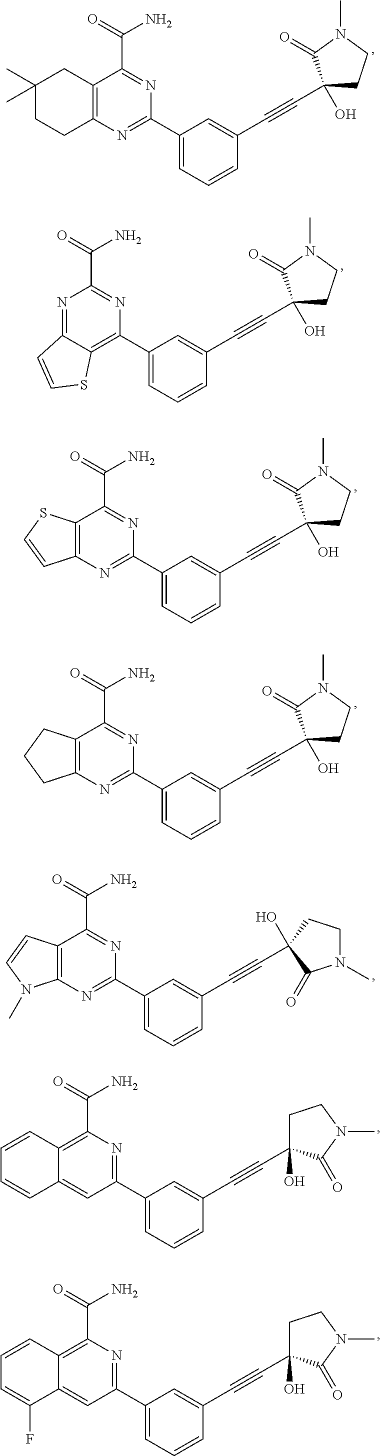

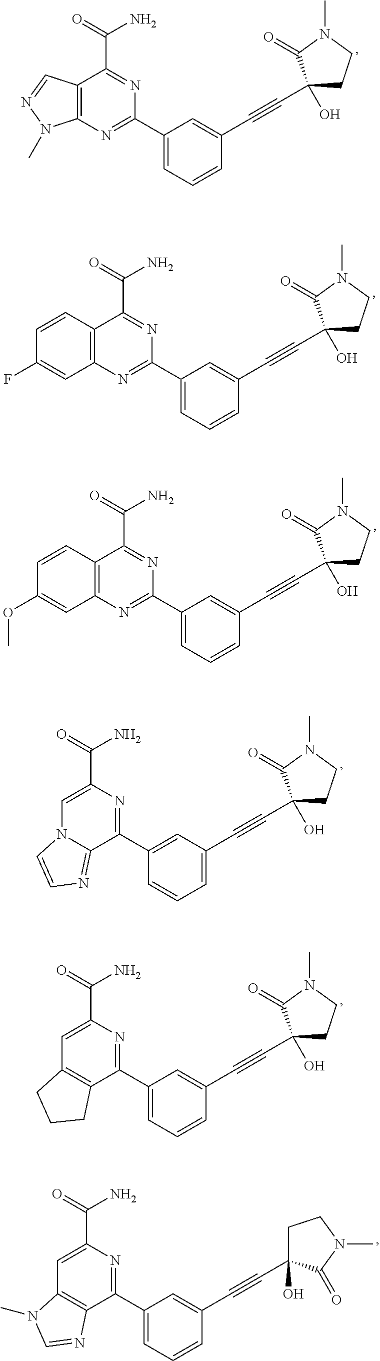

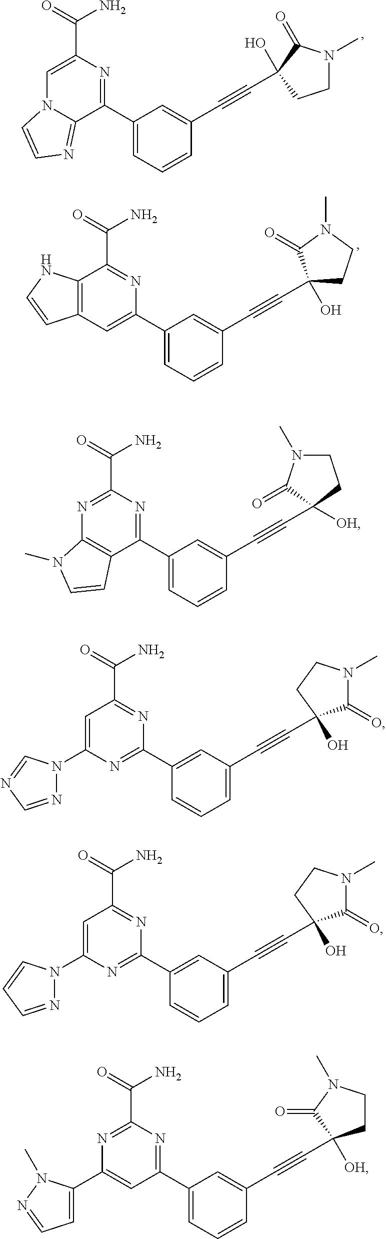

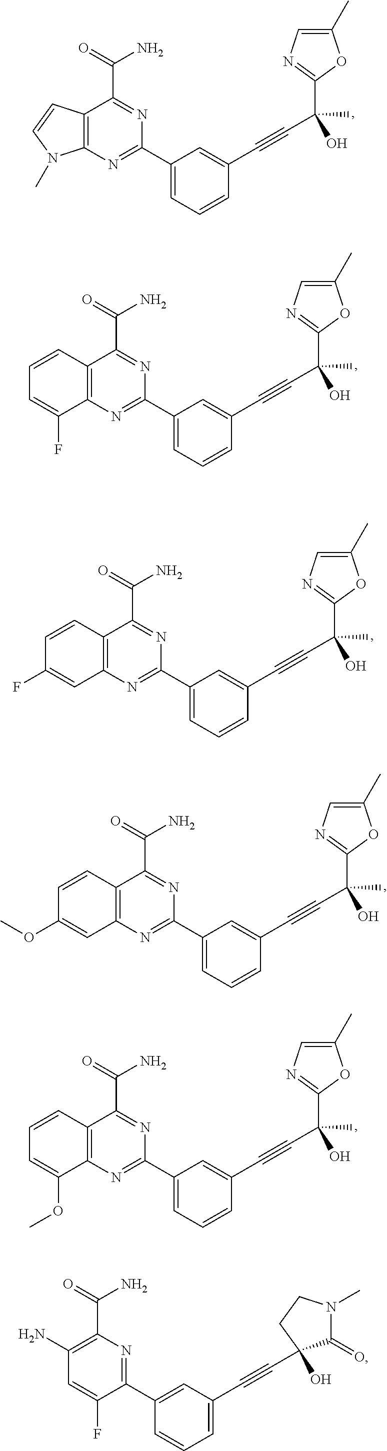

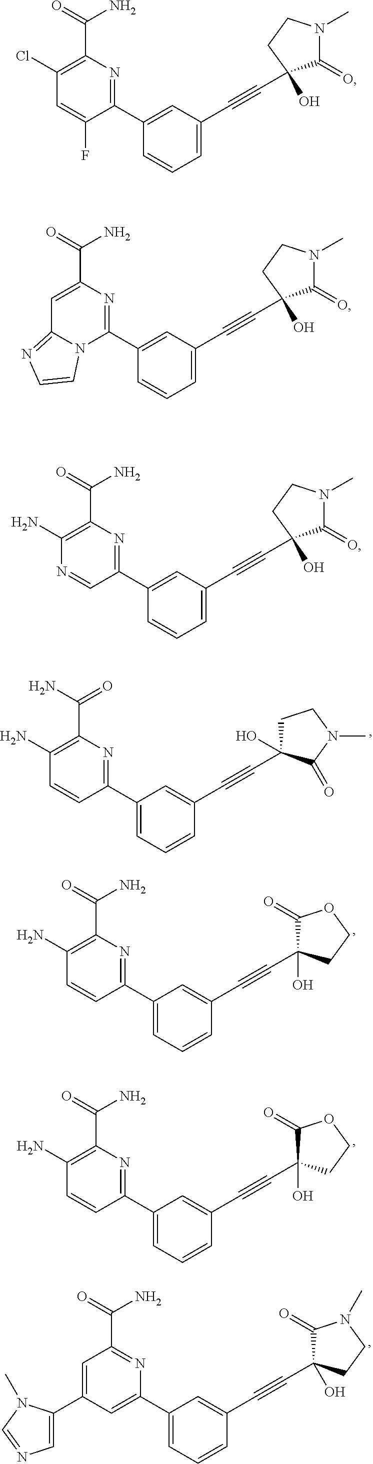

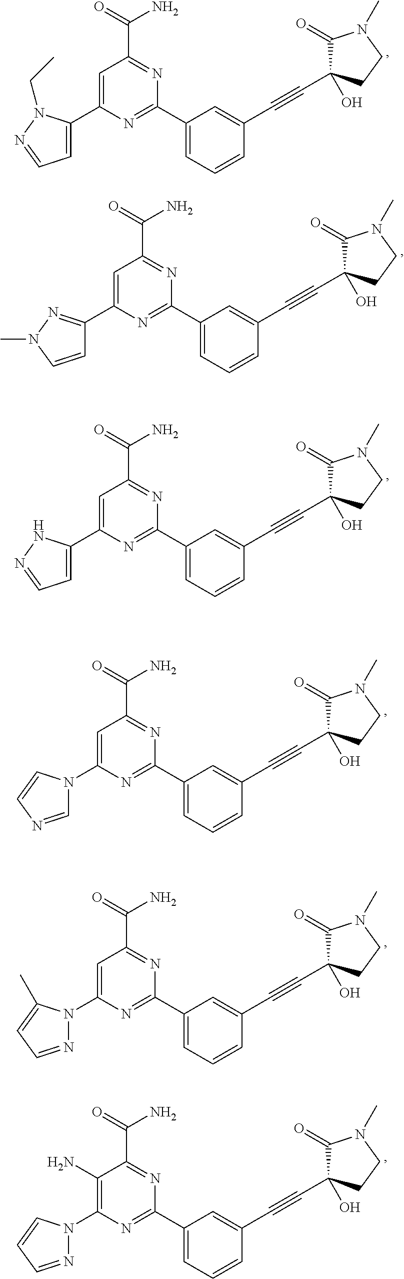 [SMILES: CCn1nccc1-c1cc(C(N)=O)nc(-c2cccc(C#C[C@]3(O)CCN(C)C3=O)c2)n1 ; CN1CC[C@@](O)(C#Cc2cccc(-c3nc(C(N)=O)cc(-c4ccn(C)n4)n3)c2)C1=O ; CN1CC[C@@](O)(C#Cc2cccc(-c3nc(C(N)=O)cc(-c4ccn[nH]4)n3)c2)C1=O ; CN1CC[C@@](O)(C#Cc2cccc(-c3nc(C(N)=O)cc(-n4ccnc4)n3)c2)C1=O ; Cc1ccnn1-c1cc(C(N)=O)nc(-c2cccc(C#C[C@]3(O)CCN(C)C3=O)c2)n1 ; CN1CC[C@@](O)(C#Cc2cccc(-c3nc(C(N)=O)c(N)c(-n4cccn4)n3)c2)C1=O]

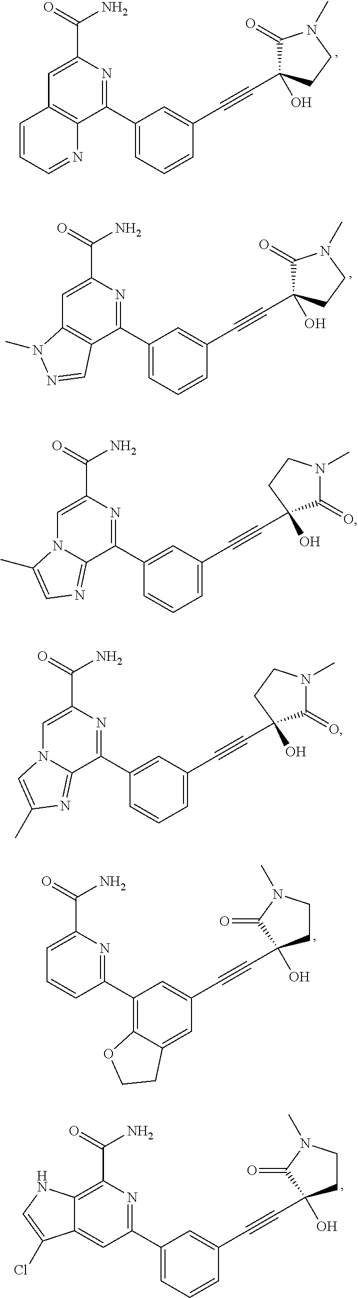 [SMILES: CN1CC[C@@](O)(C#Cc2cccc(-c3nc(C(N)=O)cc4cccnc34)c2)C1=O ; CN1CC[C@@](O)(C#Cc2cccc(-c3nc(C(N)=O)cc4c3cnn4C)c2)C1=O ; Cc1cnc2c(-c3cccc(C#C[C@]4(O)CCN(C)C4=O)c3)nc(C(N)=O)cn12 ; Cc1cn2cc(C(N)=O)nc(-c3cccc(C#C[C@]4(O)CCN(C)C4=O)c3)c2n1 ; CN1CC[C@@](O)(C#Cc2cc3c(c(-c4cccc(C(N)=O)n4)c2)OCC3)C1=O ; CN1CC[C@@](O)(C#Cc2cccc(-c3cc4c(Cl)c[nH]c4c(C(N)=O)n3)c2)C1=O]

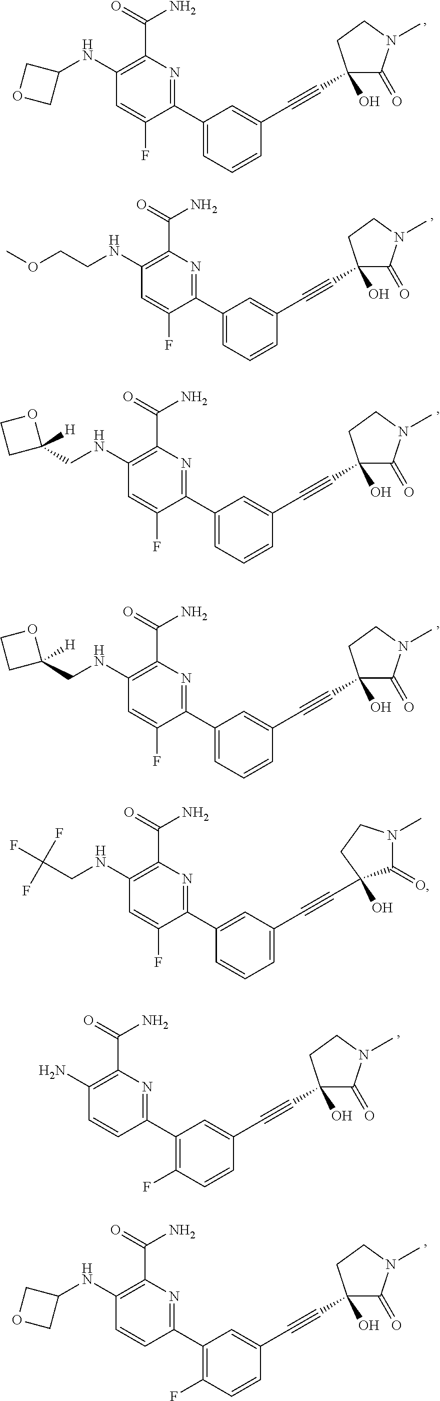

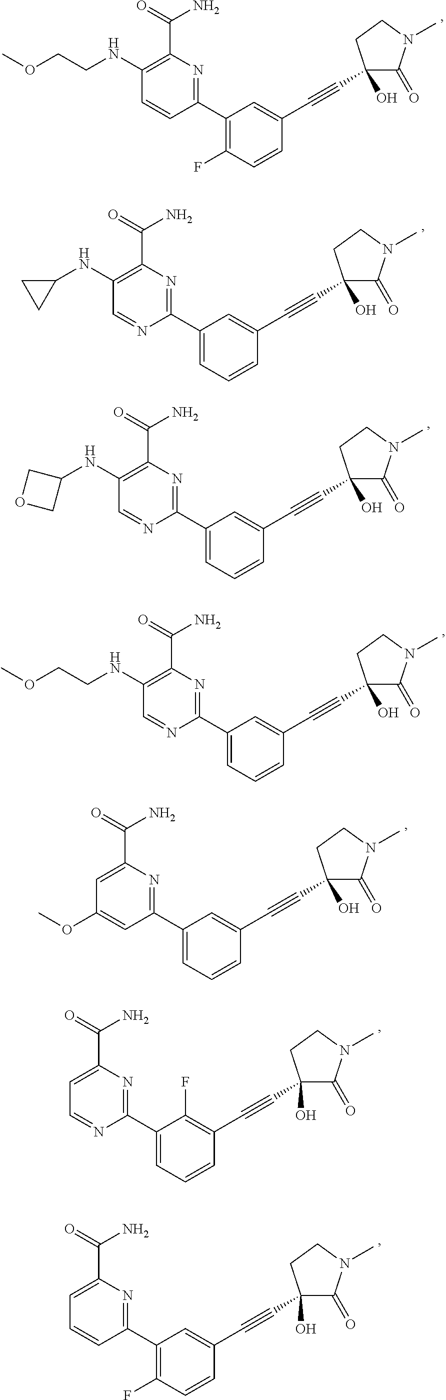

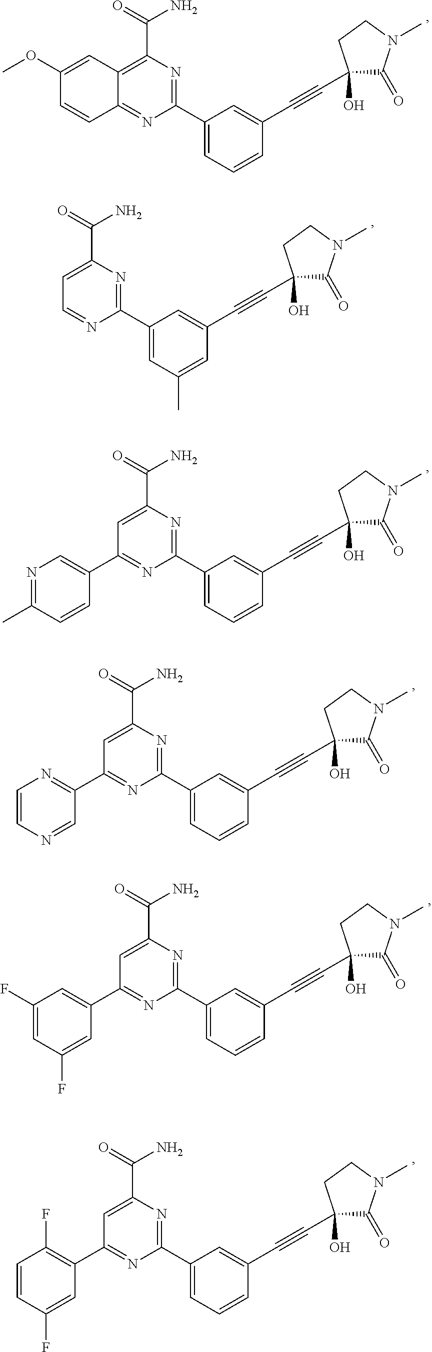

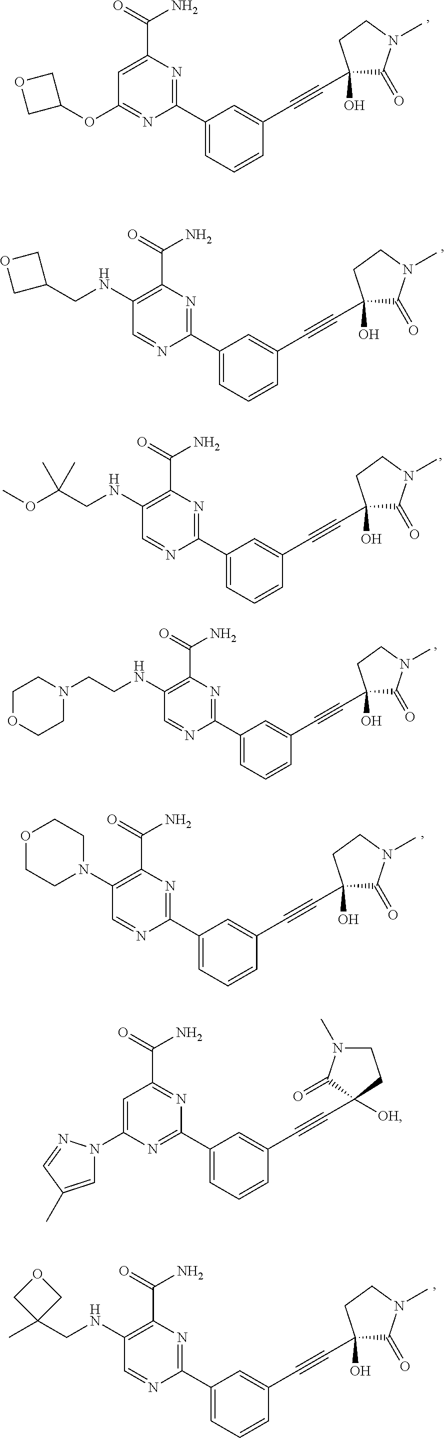

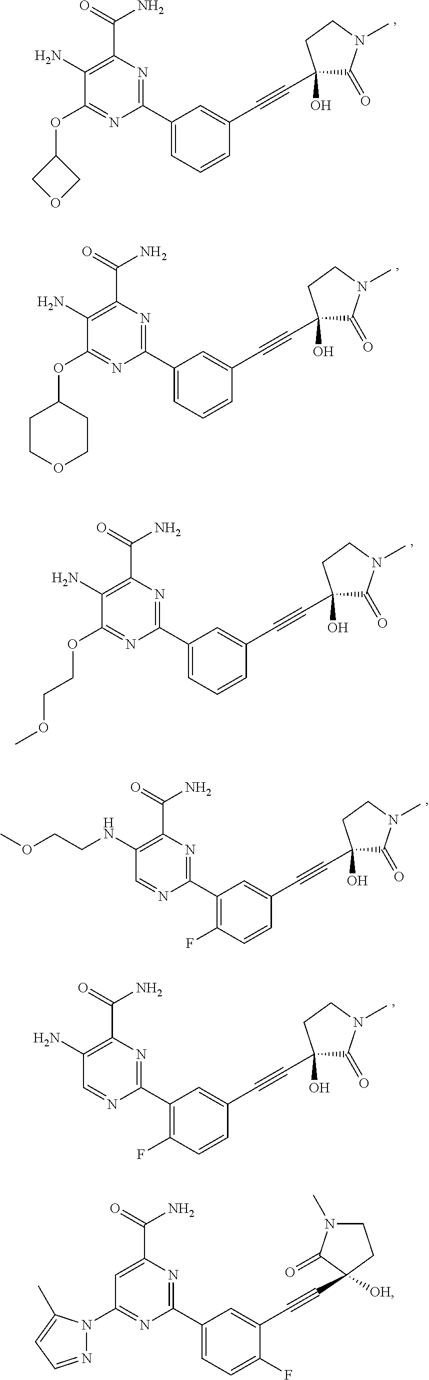

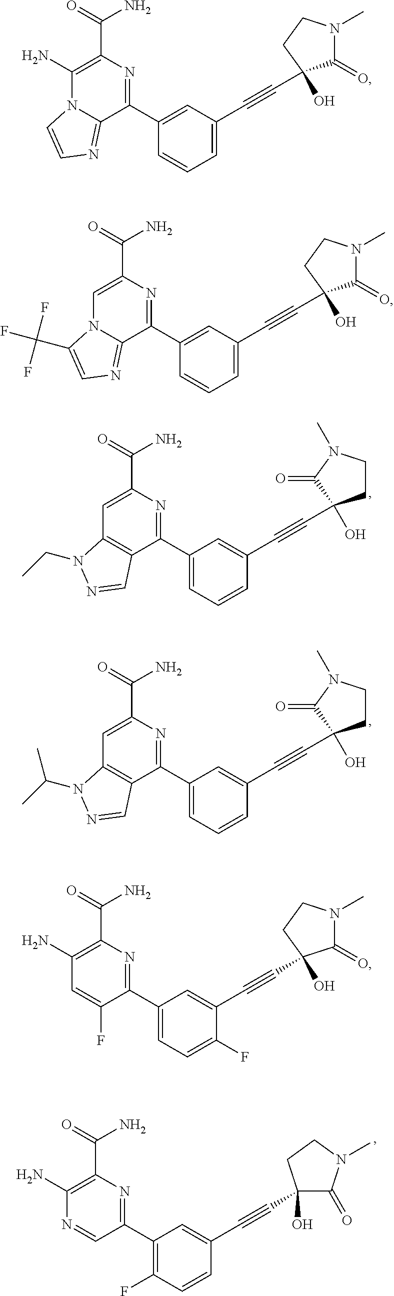

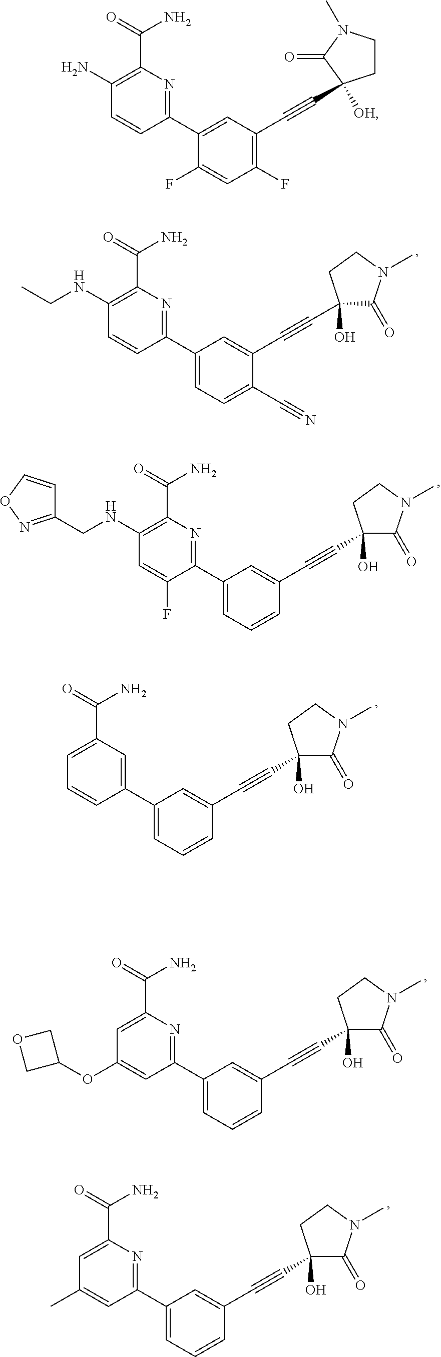

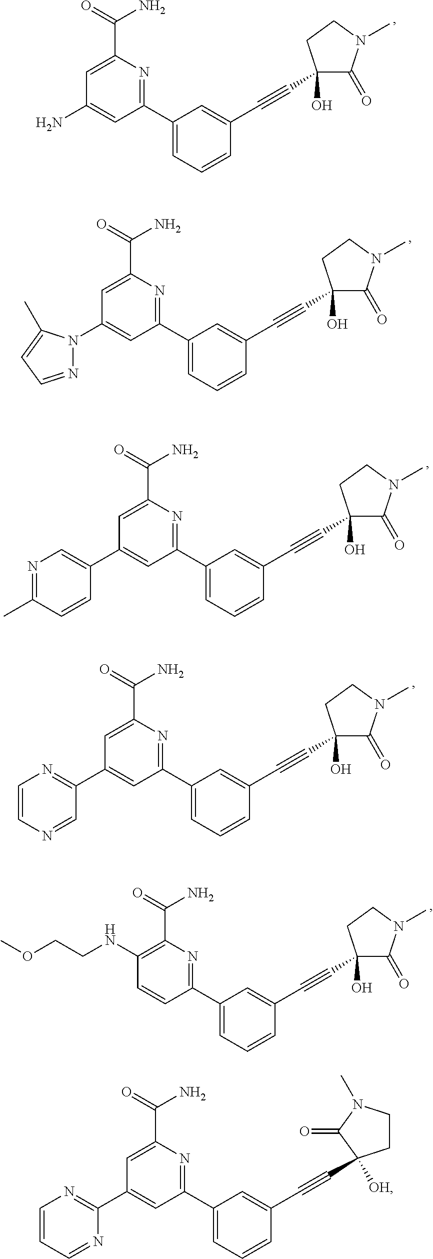

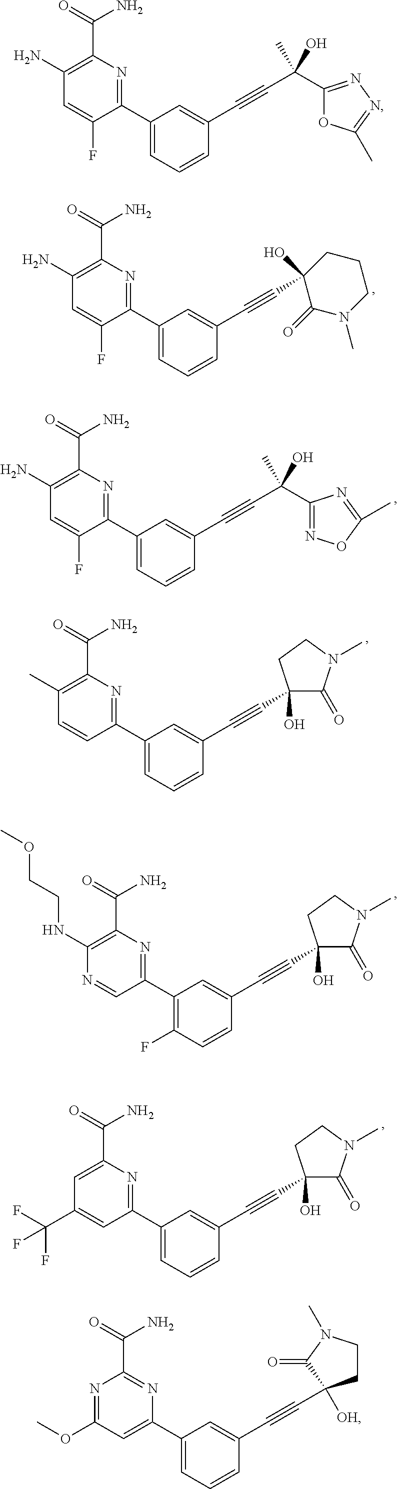

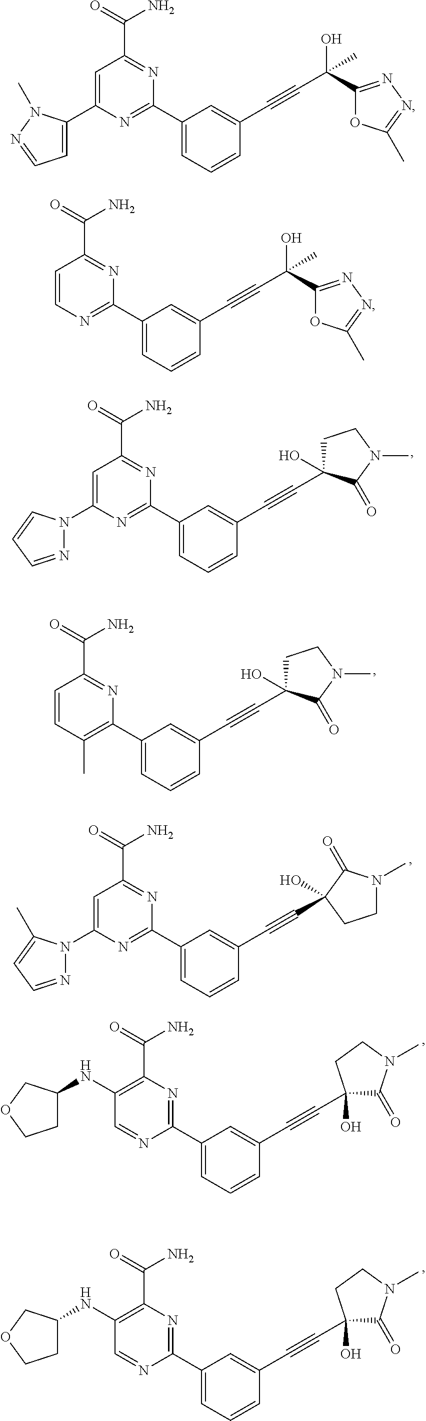

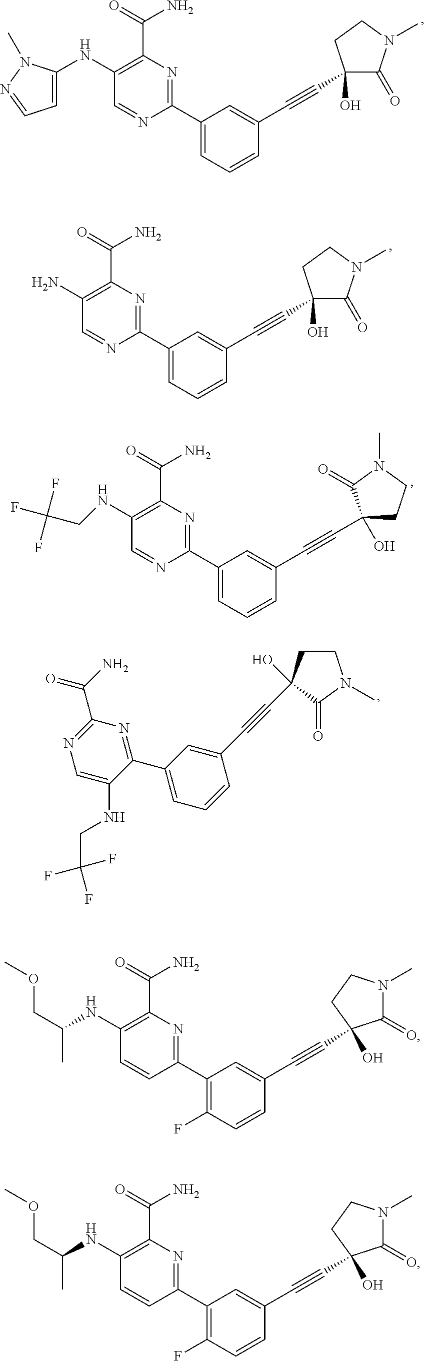

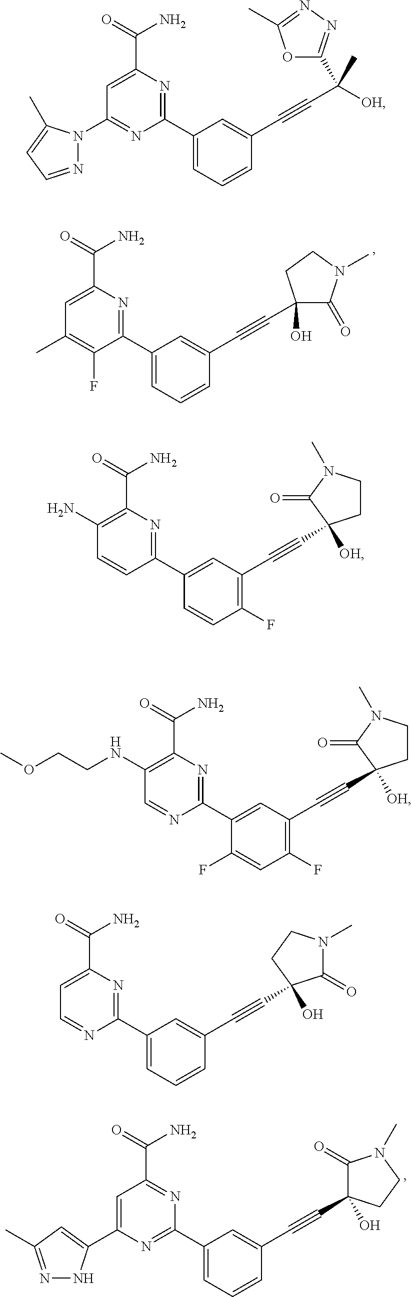

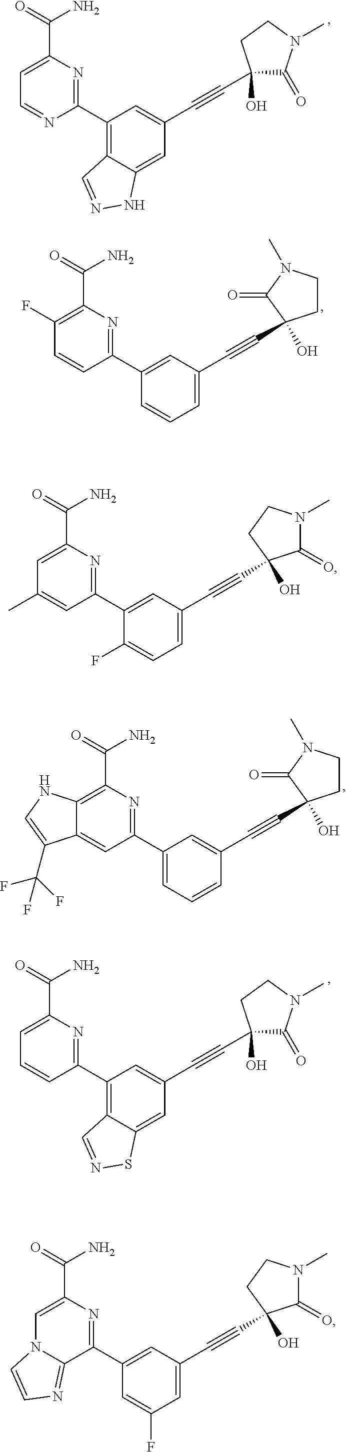

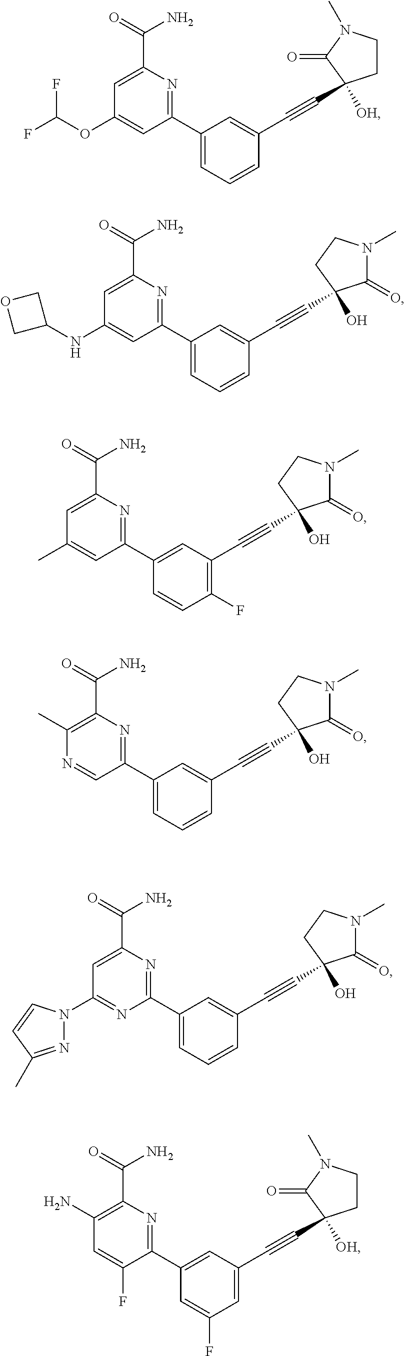

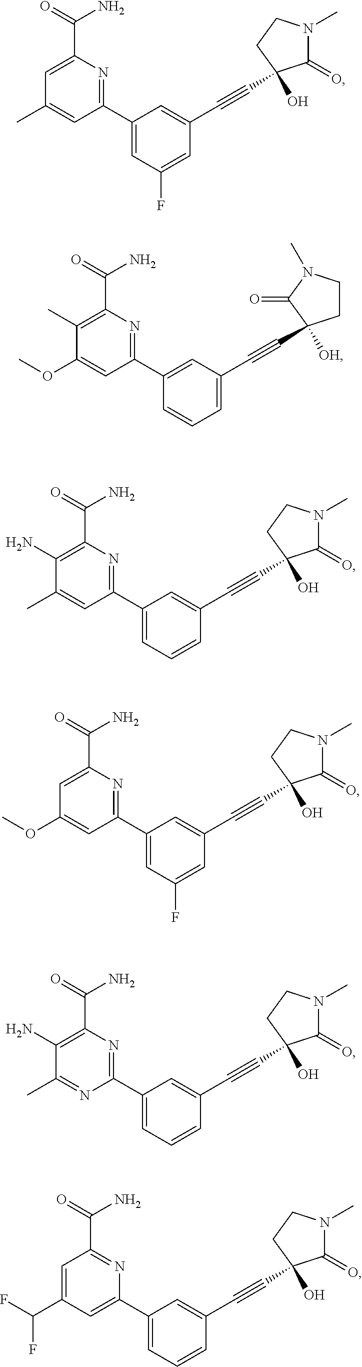

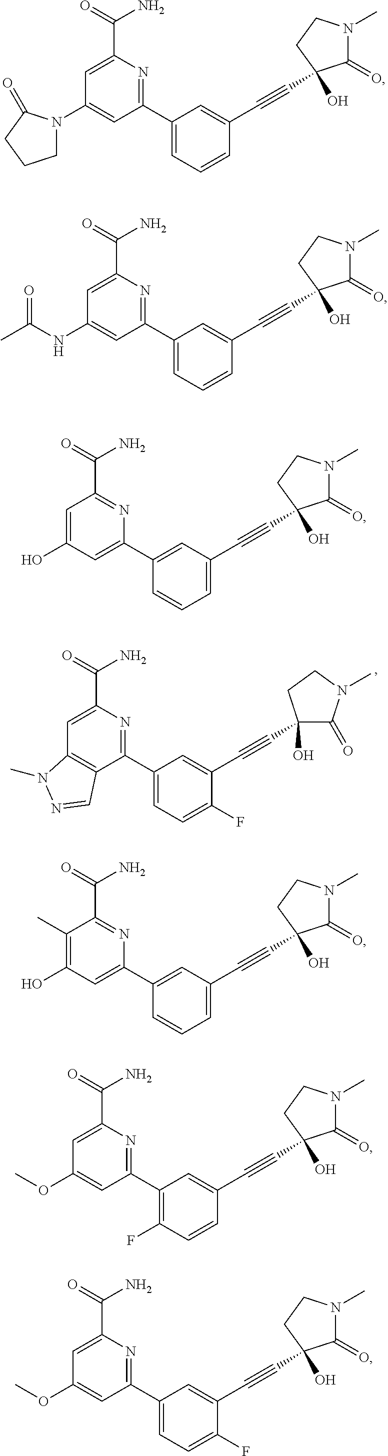

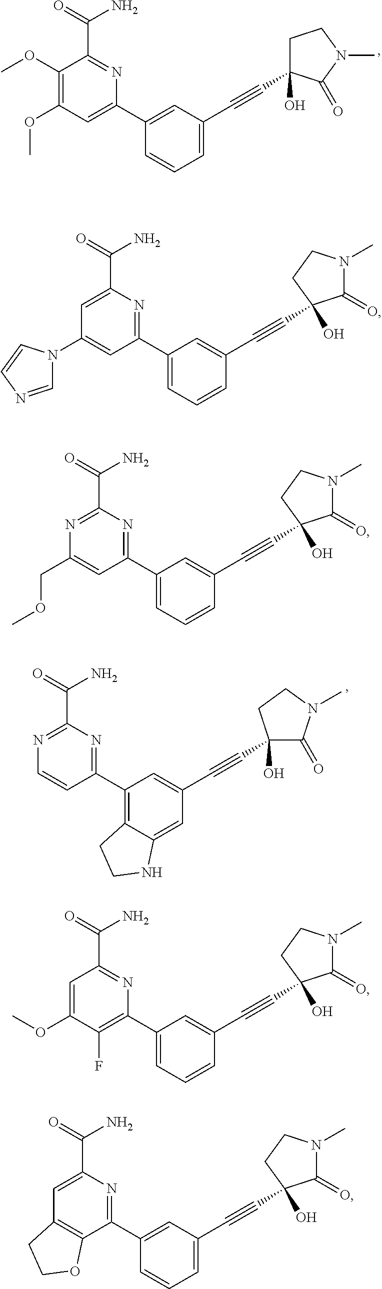

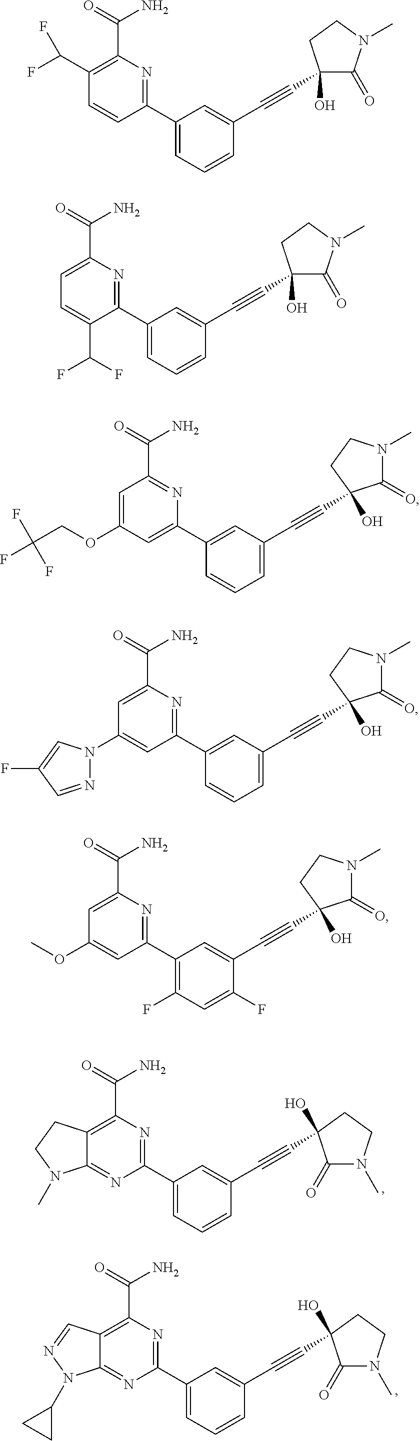

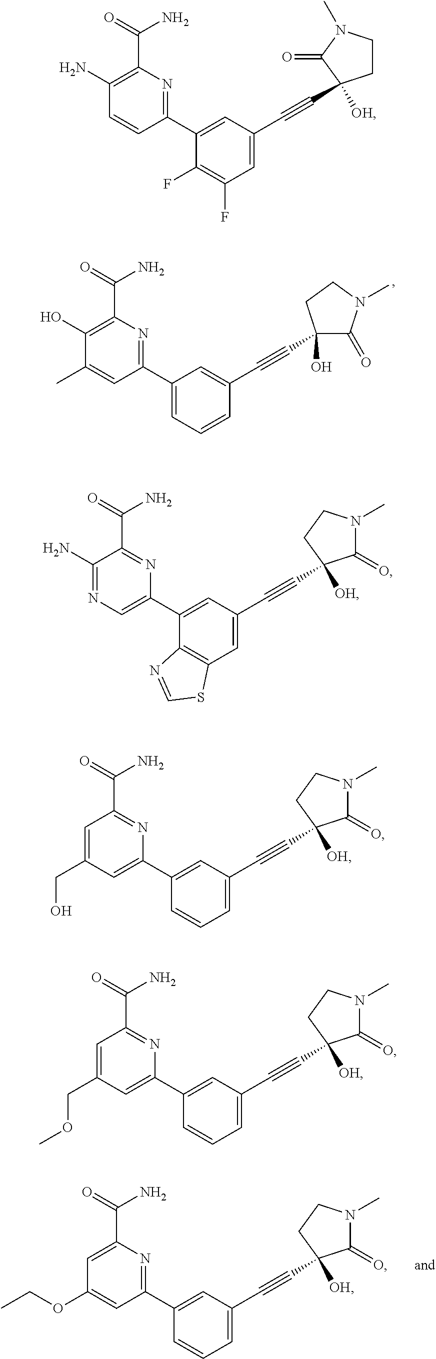

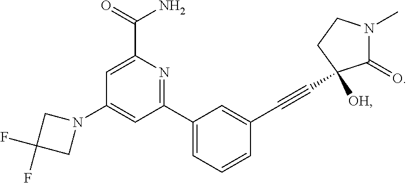 [SMILES: CN1CC[C@@](O)(C#Cc2cccc(-c3cc(N4CC(F)(F)C4)cc(C(N)=O)n3)c2)C1=O]

Some embodiments provide a pharmaceutical composition comprising a compound of the present invention and a pharmaceutically acceptable carrier, diluent or excipient. A compound or pharmaceutical composition described herein can be used in therapy, such as the treatment of an inflammatory condition (e.g., lupus, such as systemic lupus erythematosus, extra-renal lupus, or lupus nephritis, COPD, rhinitis, multiple sclerosis, IBD, arthritis, rheumatoid arthritis, dermatitis, endometriosis and transplant rejection). Also provided is the use of a compound or a pharmaceutical composition described herein for the preparation of a medicament for the treatment of an inflammatory condition (e.g., lupus, such as systemic lupus erythematosus, extra-renal lupus, or lupus nephritis, COPD, rhinitis, multiple sclerosis, IBD, arthritis, rheumatoid arthritis, dermatitis, endometriosis and transplant rejection).

Also provided is a method for the treatment of an inflammatory condition in a patient, comprising administering an effective amount of a compound or pharmaceutical composition as described herein to the patient. The inflammatory condition can be selected from the group consisting of lupus, such as systemic lupus erythematosus, extra-renal lupus, or lupus nephritis, COPD, rhinitis, multiple sclerosis, IBD, arthritis, rheumatoid arthritis, dermatitis, endometriosis and transplant rejection.

Further provided is a method of preparing a compound of Formula (0),

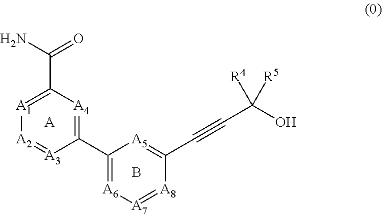

wherein A₁-A₈, R⁴ and R⁵ are as defined above, comprising: contacting a compound of Formula (A):

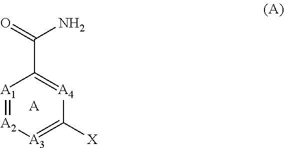

wherein X is Cl, Br or I, with a compound of Formula (B)

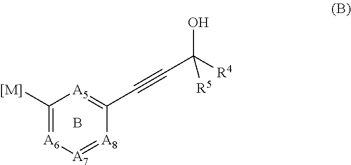

wherein [M] is a boronic acid, a boronic ester, or a trifluoroborate salt, in the presence of (a) a palladium(0) catalyst and (b) a base under Suzuki reaction conditions to yield a compound of Formula (0). Persons of skill in the art are familiar with Suzuki reactions and the reagents employed in such reactions. See, e.g., Suzuki, J. Organometallic Chem., 576:147-168 (1999). Non-limiting examples of palladium catalysts include Pd(PPh₃)₄, Pd(OAc)₂ and Pd(PPh₃)₂Cl₂. Non-limiting examples of bases include sodium carbonate, potassium carbonate and cesium carbonate, or mixtures thereof. The reaction can be carried out in a variety of organic solvents including toluene, THF, dioxane, 1,2-dichloroethane, DMF, DMSO and acetonitrile. Reaction temperatures vary depending on conditions but typically range from room temperature to 150° C.

In some embodiments, the invention provides a compound of Table 1:

TABLE 1

| Ex. | Structure | Name |
|---|---|---|
| 1 | 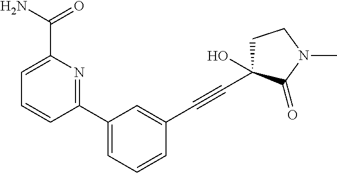 [SMILES: CN1CC[C@@](O)(C#Cc2cccc(-c3cccc(C(N)=O)n3)c2)C1=O] | 6-[3-[2-[(3R)-3-hydroxy-1- methyl-2-oxo-pyrrolidin-3- yl]ethynyl]phenyl]pyridine-2- carboxamide |
| 2 | 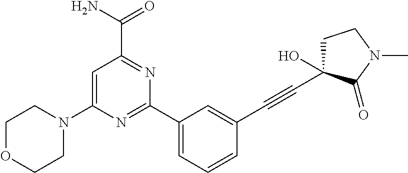 [SMILES: CN1CC[C@@](O)(C#Cc2cccc(-c3nc(C(N)=O)cc(N4CCOCC4)n3)c2)C1=O] | 2-[3-[2-[(3R)-3-hydroxy-1-methyl- 2-oxo-pyrrolidin-3-yl]ethynyl] phenyl]-6-morpholino-pyrimidine- 4-carboxamide |
| 3 | 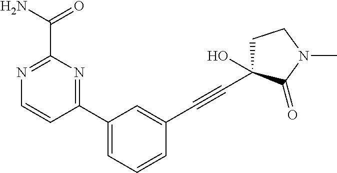 [SMILES: CN1CC[C@@](O)(C#Cc2cccc(-c3ccnc(C(N)=O)n3)c2)C1=O] | 4-[3-[2-[(3R)-3-hydroxy-1-methyl- 2-oxo-pyrrolidin-3-yl]ethynyl] phenyl]pyrimidine-2-carboxamide |
| 4 | 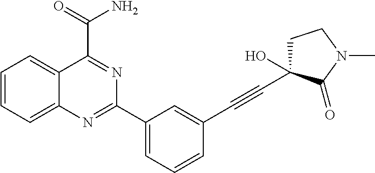 [SMILES: CN1CC[C@@](O)(C#Cc2cccc(-c3nc(C(N)=O)c4ccccc4n3)c2)C1=O] | 2-[3-[2-[(3R)-3-hydroxy-1-methyl- 2-oxo-pyrrolidin-3-yl]ethynyl] phenyl]quinazoline-4-carboxamide |
| 5 | 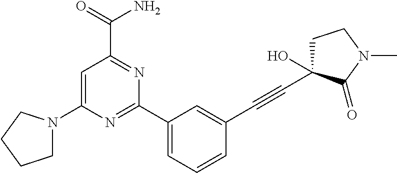 [SMILES: CN1CC[C@@](O)(C#Cc2cccc(-c3nc(C(N)=O)cc(N4CCCC4)n3)c2)C1=O] | 2-[3-[2-[(3R)-3-hydroxy-1-methyl- 2-oxo-pyrrolidin-3-yl]ethynyl] phenyl]-6-pyrrolidin-1-yl- pyrimidine-4-carboxamide |
| 6 | 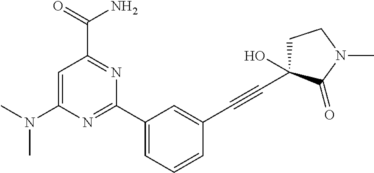 | 6-(dimethylamino)-2-[3-[2-[(3R)- 3-hydroxy-1-methyl-2-oxo- pyrrolidin-3-yl]ethynyl]phenyl] pyrimidine-4-carboxamide |
| 7 | 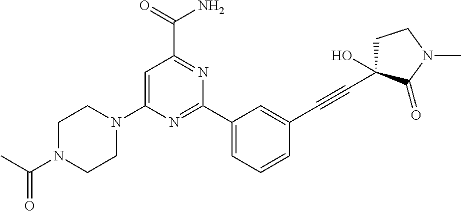 [SMILES: CC(=O)N1CCN(c2cc(C(N)=O)nc(-c3cccc(C#C[C@]4(O)CCN(C)C4=O)c3)n2)CC1] | 6-(4-acetylpiperazin-1-yl)-2-[3- [2-[(3R)-3-hydroxy-1-methyl-2- oxo-pyrrolidin-3-yl]ethynyl] phenyl]pyrimidine-4-carboxamide |
| 8 | 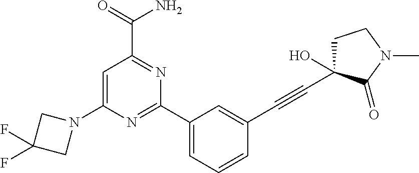 [SMILES: CN1CC[C@@](O)(C#Cc2cccc(-c3nc(C(N)=O)cc(N4CC(F)(F)C4)n3)c2)C1=O] | 6-(3,3-difluoroazetidin-1-yl)-2- [3-[2-[(3R)-3-hydroxy-1-methyl- 2-oxo-pyrrolidin-3-yl]ethynyl] phenyl]pyrimidine-4-carboxamide |
| 9 | 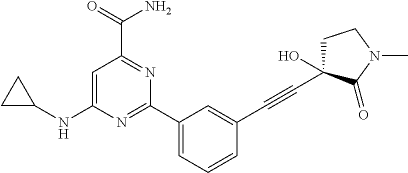 [SMILES: CN1CC[C@@](O)(C#Cc2cccc(-c3nc(NC4CC4)cc(C(N)=O)n3)c2)C1=O] | 6-(cyclopropylamino)-2-[3-[2- [(3R)-3-hydroxy-1-methyl-2-oxo- pyrrolidin-3-yl]ethynyl]phenyl] pyrimidine-4-carboxamide |
| 10 | 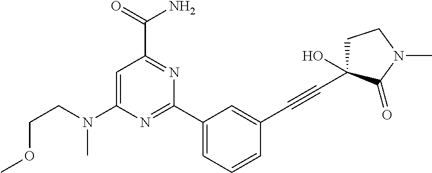 | 2-[3-[2-[(3R)-3-hydroxy-1- methyl-2-oxo-pyrrolidin-3- yl]ethynyl]phenyl]-6-[2- methoxyethyl(methyl)amino] pyrimidine-4-carboxamide |
| 11 | 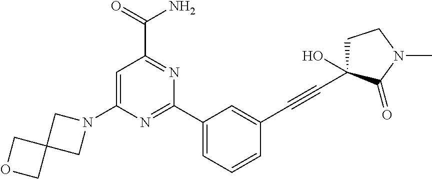 [SMILES: CN1CC[C@@](O)(C#Cc2cccc(-c3nc(C(N)=O)cc(N4CC5(COC5)C4)n3)c2)C1=O] | 2-[3-[2-[(3R)-3-hydroxy-1- methyl-2-oxo-pyrrolidin-3-yl] ethynyl]phenyl]-6-(6-oxa-2- azaspiro[3.3]heptan-2-yl) pyrimidine-4-carboxamide |
| 12 | 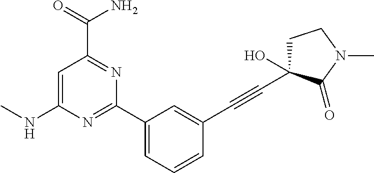 | 2-[3-[2-[(3R)-3-hydroxy-1- methyl-2-oxo-pyrrolidin-3- yl]ethynyl]phenyl]-6- (methylamino)pyrimidine- 4-carboxamide |
| 13 | 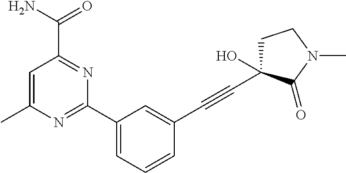 | 2-[3-[2-[(3R)-3-hydroxy-1- methyl-2-oxo-pyrrolidin-3- yl]ethynyl]phenyl]-6-methyl- pyrimidine-4-carboxamide |
| 14 | 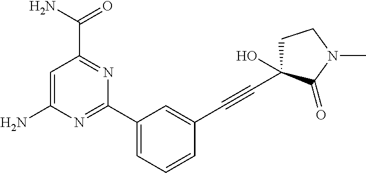 [SMILES: CN1CC[C@@](O)(C#Cc2cccc(-c3nc(N)cc(C(N)=O)n3)c2)C1=O] | 6-amino-2-[3-[2-[(3R)-3- hydroxy-1-methyl-2-oxo- pyrrolidin-3-yl]ethynyl] phenyl]pyrimidine-4- carboxamide |
| 15 | 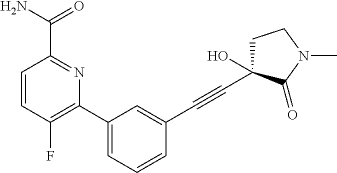 [SMILES: CN1CC[C@@](O)(C#Cc2cccc(-c3nc(C(N)=O)ccc3F)c2)C1=O] | 5-fluoro-6-[3-[2-[(3R)-3- hydroxy-1-methyl-2-oxo- pyrrolidin-3-yl]ethynyl]phenyl] pyridine-2-carboxamide |
| 16 | 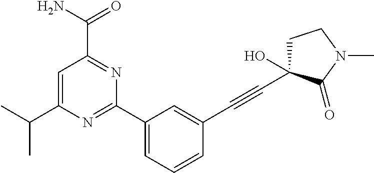 [SMILES: CC(C)c1cc(C(N)=O)nc(-c2cccc(C#C[C@]3(O)CCN(C)C3=O)c2)n1] | 2-[3-[2-[(3R)-3-hydroxy-1- methyl-2-oxo-pyrrolidin-3- yl]ethynyl]phenyl]-6-isopropyl- pyrimidine-4-carboxamide |
| 17 | 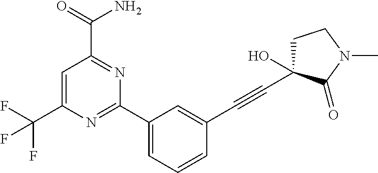 [SMILES: CN1CC[C@@](O)(C#Cc2cccc(-c3nc(C(N)=O)cc(C(F)(F)F)n3)c2)C1=O] | 2-[3-[2-[(3R)-3-hydroxy-1- methyl-2-oxo-pyrrolidin-3- yl]ethynyl]phenyl]-6- (trifluoromethyl)pyrimidine-4- carboxamide |
| 18 | 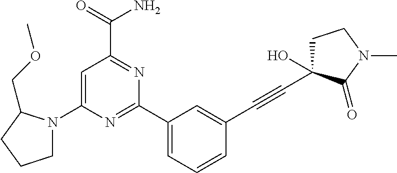 [SMILES: COCC1CCCN1c1cc(C(N)=O)nc(-c2cccc(C#C[C@]3(O)CCN(C)C3=O)c2)n1] | 2-[3-[2-[(3R)-3-hydroxy-1- methyl-2-oxo-pyrrolidin-3- yl]ethynyl]phenyl]-6-[2- (methoxymethyl)pyrrolidin-1- yl]pyrimidine-4-carboxamide |
| 19 | 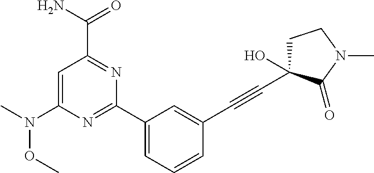 | 2-[3-[2-[(3R)-3-hydroxy-1- methyl-2-oxo-pyrrolidin-3- yl]ethynyl]phenyl]-6-[methoxy (methyl)amino]pyrimidine-4- carboxamide |
| 20 | 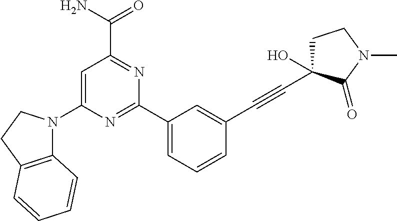 [SMILES: CN1CC[C@@](O)(C#Cc2cccc(-c3nc(C(N)=O)cc(N4CCc5ccccc54)n3)c2)C1=O] | 2-[3-[2-[(3R)-3-hydroxy-1- methyl-2-oxo-pyrrolidin-3- yl]ethynyl]phenyl]-6-indolin-1- yl-pyrimidine-4-carboxamide |
| 21 | 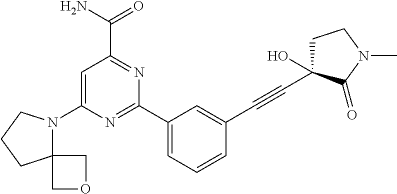 [SMILES: CN1CC[C@@](O)(C#Cc2cccc(-c3nc(C(N)=O)cc(N4CCCC45COC5)n3)c2)C1=O] | 2-[3-[2-[(3R)-3-hydroxy-1- methyl-2-oxo-pyrrolidin-3- yl]ethynyl]phenyl]-6-(2-oxa-8- azaspiro[3.4]octan-8-yl) pyrimidine-4-carboxamide |
| 22 | 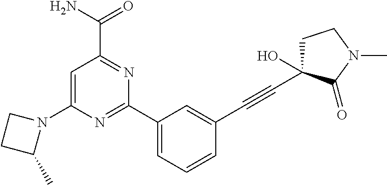 [SMILES: C[C@@H]1CCN1c1cc(C(N)=O)nc(-c2cccc(C#C[C@]3(O)CCN(C)C3=O)c2)n1] | 2-[3-[2-[(3R)-3-hydroxy-1- methyl-2-oxo-pyrrolidin-3-yl] ethynyl]phenyl]-6-[(2R)-2- methylazetidin-1-yl]pyrimidine- 4-carboxamide |
| 23 | 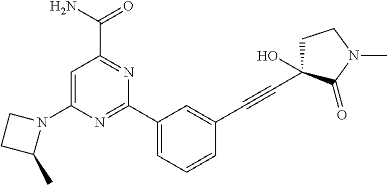 [SMILES: C[C@H]1CCN1c1cc(C(N)=O)nc(-c2cccc(C#C[C@]3(O)CCN(C)C3=O)c2)n1] | 2-[3-[2-[(3R)-3-hydroxy-1- methyl-2-oxo-pyrrolidin-3-yl] ethynyl]phenyl]-6-[(2S)-2- methylazetidin-1-yl] pyrimidine-4-carboxamide |
| 24 | 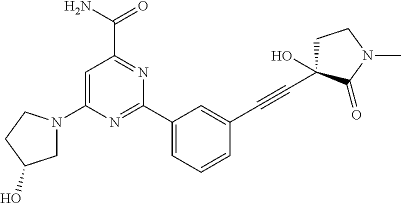 [SMILES: CN1CC[C@@](O)(C#Cc2cccc(-c3nc(C(N)=O)cc(N4CC[C@@H](O)C4)n3)c2)C1=O] | 2-[3-[2-[(3R)-3-hydroxy-1- methyl-2-oxo-pyrrolidin-3- yl]ethynyl]phenyl]-6-[(3R)- 3-hydroxypyrrolidin-1-yl] pyrimidine-4-carboxamide |
| 25 | 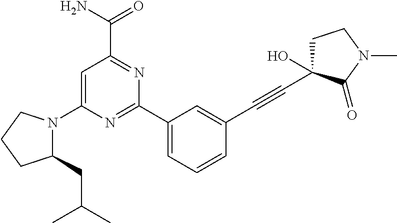 [SMILES: CC(C)C[C@H]1CCCN1c1cc(C(N)=O)nc(-c2cccc(C#C[C@]3(O)CCN(C)C3=O)c2)n1] | 2-[3-[2-[(3R)-3-hydroxy-1- methyl-2-oxo-pyrrolidin-3- yl]ethynyl]phenyl]-6-[(2R)- 2-isobutylpyrrolidin-1-yl] pyrimidine-4-carboxamide |
| 26 | 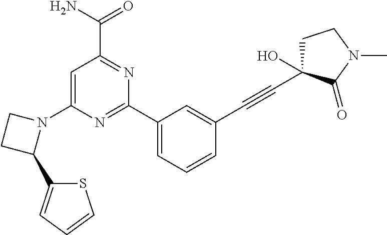 [SMILES: CN1CC[C@@](O)(C#Cc2cccc(-c3nc(C(N)=O)cc(N4CC[C@@H]4c4cccs4)n3)c2)C1=O] | 2-[3-[2-[(3R)-3-hydroxy-1- methyl-2-oxo-pyrrolidin-3- yl]ethynyl]phenyl]-6-[(2R)- 2-(2-thienyl)azetidin-1-yl] pyrimidine-4-carboxamide |
| 27 | 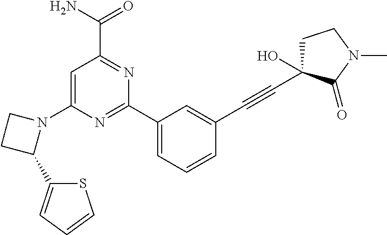 [SMILES: CN1CC[C@@](O)(C#Cc2cccc(-c3nc(C(N)=O)cc(N4CC[C@H]4c4cccs4)n3)c2)C1=O] | 2-[3-[2-[(3R)-3-hydroxy-1- methyl-2-oxo-pyrrolidin-3- yl]ethynyl]phenyl]-6-[(2S)- 2-(2-thienyl)azetidin-1- yl]pyrimidine-4-carboxamide |
| 28 | 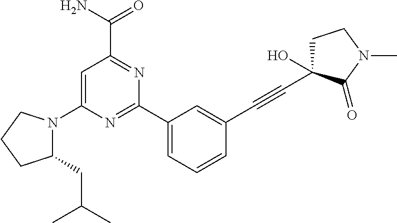 [SMILES: CC(C)C[C@@H]1CCCN1c1cc(C(N)=O)nc(-c2cccc(C#C[C@]3(O)CCN(C)C3=O)c2)n1] | 2-[3-[2-[(3R)-3-hydroxy-1- methyl-2-oxo-pyrrolidin-3- yl]ethynyl]phenyl]-6-[(2S)- 2-isobutylpyrrolidin-1-yl] pyrimidine-4-carboxamide |
| 29 | 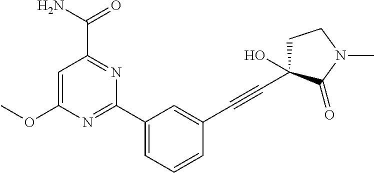 | 2-[3-[2-[(3R)-3-hydroxy-1- methyl-2-oxo-pyrrolidin-3- yl]ethynyl]phenyl]-6-methoxy- pyrimidine-4-carboxamide |
| 30 | 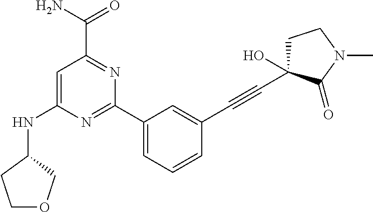 [SMILES: CN1CC[C@@](O)(C#Cc2cccc(-c3nc(N[C@H]4CCOC4)cc(C(N)=O)n3)c2)C1=O] | 2-[3-[2-[(3R)-3-hydroxy-1- methyl-2-oxo-pyrrolidin-3- yl]ethynyl]phenyl]-6-[[(3S)- tetrahydrofuran-3-yl]amino] pyrimidine-4-carboxamide |
| 31 | 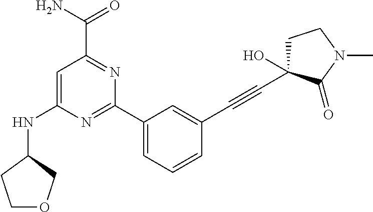 [SMILES: CN1CC[C@@](O)(C#Cc2cccc(-c3nc(N[C@@H]4CCOC4)cc(C(N)=O)n3)c2)C1=O] | 2-[3-[2-[(3R)-3-hydroxy-1- methyl-2-oxo-pyrrolidin-3- yl]ethynyl]phenyl]-6-[[(3R)- tetrahydrofuran-3-yl]amino] pyrimidine-4-carboxamide |
| 32 | 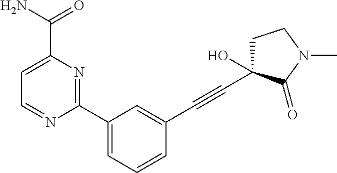 [SMILES: CN1CC[C@@](O)(C#Cc2cccc(-c3nccc(C(N)=O)n3)c2)C1=O] | 2-[3-[2-[(3R)-3-hydroxy-1- methyl-2-oxo-pyrrolidin-3- yl]ethynyl]phenyl] pyrimidine-4-carboxamide |
| 33 | 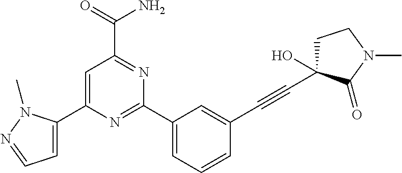 [SMILES: CN1CC[C@@](O)(C#Cc2cccc(-c3nc(C(N)=O)cc(-c4ccnn4C)n3)c2)C1=O] | 2-[3-[2-[(3R)-3-hydroxy-1- methyl-2-oxo-pyrrolidin-3- yl]ethynyl]phenyl]-6-(2- methylpyrazol-3-yl) pyrimidine-4-carboxamide |
| 34 | 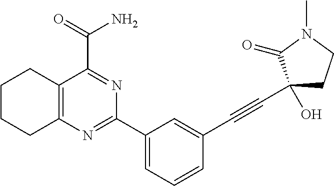 [SMILES: CN1CC[C@@](O)(C#Cc2cccc(-c3nc4c(c(C(N)=O)n3)CCCC4)c2)C1=O] | 2-[3-[2-[(3R)-3-hydroxy-1- methyl-2-oxo-pyrrolidin-3- yl]ethynyl]phenyl]-5,6,7,8- tetrahydroquinazoline-4- carboxamide |
| 35 | 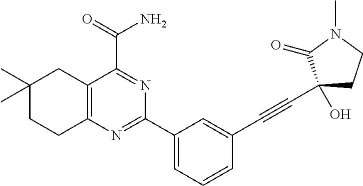 [SMILES: CN1CC[C@@](O)(C#Cc2cccc(-c3nc4c(c(C(N)=O)n3)CC(C)(C)CC4)c2)C1=O] | 2-[3-[2-[(3R)-3-hydroxy-1- methyl-2-oxo-pyrrolidin-3- yl]ethynyl]phenyl]-6,6- dimethyl-7,8-dihydro-5H- quinazoline-4-carboxamide |
| 36 | 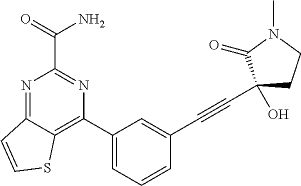 [SMILES: CN1CC[C@@](O)(C#Cc2cccc(-c3nc(C(N)=O)nc4ccsc34)c2)C1=O] | 4-[3-[2-[(3R)-3-hydroxy-1- methyl-2-oxo-pyrrolidin-3- yl]ethynyl]phenyl]thieno[3,2- d]pyrimidine-2-carboxamide |
| 37 | 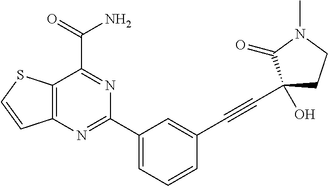 [SMILES: CN1CC[C@@](O)(C#Cc2cccc(-c3nc(C(N)=O)c4sccc4n3)c2)C1=O] | 2-[3-[2-[(3R)-3-hydroxy-1- methyl-2-oxo-pyrrolidin-3- yl]ethynyl]phenyl]thieno[3,2- d]pyrimidine-4-carboxamide |
| 38 | 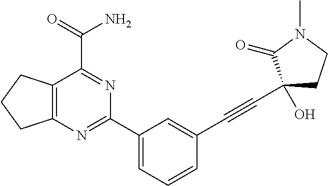 [SMILES: CN1CC[C@@](O)(C#Cc2cccc(-c3nc4c(c(C(N)=O)n3)CCC4)c2)C1=O] | 2-[3-[2-[(3R)-3-hydroxy-1- methyl-2-oxo-pyrrolidin-3- yl]ethynyl]phenyl]-6,7- dihydro-5H-cyclopenta [d]pyrimidine-4-carboxamide |
| 39 | 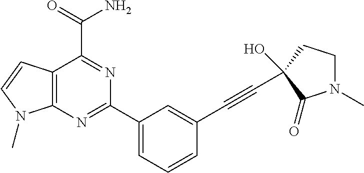 [SMILES: CN1CC[C@@](O)(C#Cc2cccc(-c3nc(C(N)=O)c4ccn(C)c4n3)c2)C1=O] | 2-[3-[2-[(3R)-3-hydroxy-1- methyl-2-oxo-pyrrolidin-3- yl]ethynyl]phenyl]-7-methyl- pyrrolo[2,3-d] pyrimidine-4-carboxamide |
| 40 | 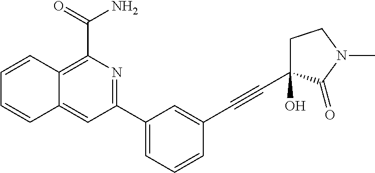 [SMILES: CN1CC[C@@](O)(C#Cc2cccc(-c3cc4ccccc4c(C(N)=O)n3)c2)C1=O] | 3-[3-[2-[(3R)-3-hydroxy-1- methyl-2-oxo-pyrrolidin-3- yl]ethynyl]phenyl] isoquinoline-1-carboxamide |
| 41 | 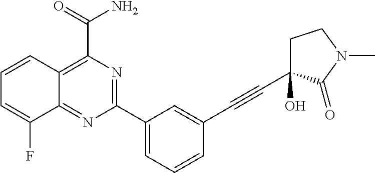 [SMILES: CN1CC[C@@](O)(C#Cc2cccc(-c3nc(C(N)=O)c4cccc(F)c4n3)c2)C1=O] | 8-fluoro-2-[3-[2-[(3R)-3- hydroxy-1-methyl-2-oxo- pyrrolidin-3-yl]ethynyl] phenyl]quinazoline-4- carboxamide |
| 42 | 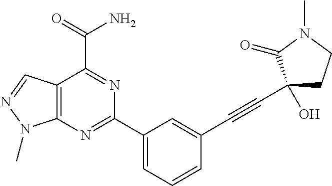 [SMILES: CN1CC[C@@](O)(C#Cc2cccc(-c3nc(C(N)=O)c4cnn(C)c4n3)c2)C1=O] | 6-(3-[2-[(3R)-3-hydroxy-1- methyl-2-oxopyrrolidin-3- yl]ethynyl]phenyl)-1-methyl- 1H-pyrazolo[3,4-d] pyrimidine-4-carboxamide |
| 43 | 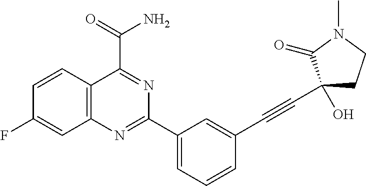 [SMILES: CN1CC[C@@](O)(C#Cc2cccc(-c3nc(C(N)=O)c4ccc(F)cc4n3)c2)C1=O] | 7-fluoro-2-(3-[2-[(3R)-3- hydroxy-1-methyl-2- oxopyrrolidin-3-yl] ethynyl]phenyl)quinazoline-4- carboxamide |
| 44 | 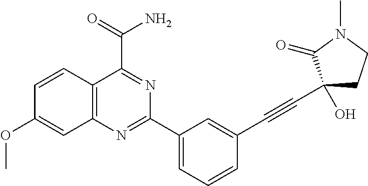 [SMILES: COc1ccc2c(C(N)=O)nc(-c3cccc(C#C[C@]4(O)CCN(C)C4=O)c3)nc2c1] | 2-(3-[2-[(3R)-3-hydroxy-1- methyl-2-oxopyrrolidin-3- yl]ethynyl]phenyl)-7- methoxyquinazoline-4- carboxamide |
| 45 | 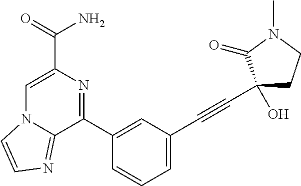 [SMILES: CN1CC[C@@](O)(C#Cc2cccc(-c3nc(C(N)=O)cn4ccnc34)c2)C1=O] | 8-(3-[2-[(3S)-3-hydroxy-1- methyl-2-oxopyrrolidin-3- yl]ethynyl]phenyl)imidazo [1,2-a]pyrazine-6- carboxamide |
| 46 | 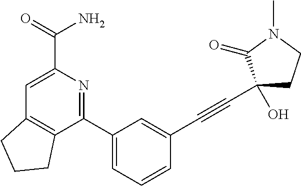 [SMILES: CN1CC[C@@](O)(C#Cc2cccc(-c3nc(C(N)=O)cc4c3CCC4)c2)C1=O] | 1-(3-[2-[(3R)-3-hydroxy-1- methyl-2-oxopyrrolidin-3-yl] ethynyl]phenyl)-5H,6H,7H- cyclopenta[c] pyridine-3-carboxamide |
| 47 | 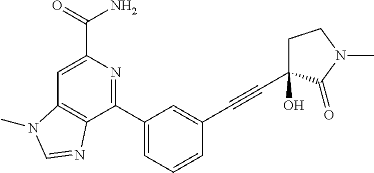 | 4-[3-[2-[(3R)-3-hydroxy-1- methyl-2-oxo-pyrrolidin- 3-yl]ethynyl]phenyl]-1- methyl-imidazo[4,5-c] pyridine-6-carboxamide |
| 48 | 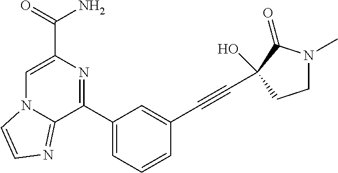 [SMILES: CN1CC[C@@](O)(C#Cc2cccc(-c3nc(C(N)=O)cn4ccnc34)c2)C1=O] | 8-[3-[2-[(3R)-3-hydroxy-1- methyl-2-oxo-pyrrolidin- 3-yl]ethynyl]phenyl] imidazo[1,2-a]pyrazine- 6-carboxamide |
| 49 | 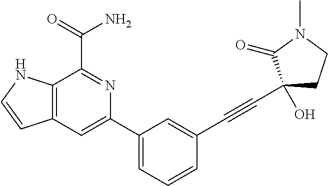 [SMILES: CN1CC[C@@](O)(C#Cc2cccc(-c3cc4cc[nH]c4c(C(N)=O)n3)c2)C1=O] | 5-[3-[2-[(3R)-3-hydroxy-1- methyl-2-oxo-pyrrolidin-3- yl]ethynyl]phenyl]-1H-pyrrolo [2,3-c]pyridine-7-carboxamide |
| 50 | 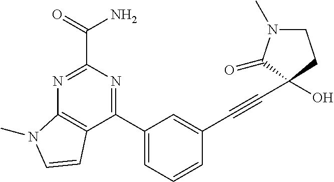 [SMILES: CN1CC[C@@](O)(C#Cc2cccc(-c3nc(C(N)=O)nc4c3ccn4C)c2)C1=O] | 4-[3-[2-[(3R)-3-hydroxy-1- methyl-2-oxo-pyrrolidin-3- yl]ethynyl]phenyl]-7-methyl- pyrrolo[2,3-d]pyrimidine- 2-carboxamide |
| 51 | 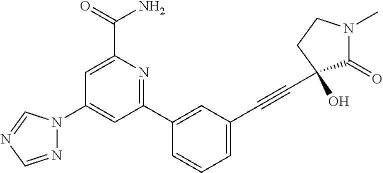 [SMILES: CN1CC[C@@](O)(C#Cc2cccc(-c3cc(-n4cncn4)cc(C(N)=O)n3)c2)C1=O] | 6-[3-[2-[(3R)-3-hydroxy-1- methyl-2-oxo-pyrrolidin-3- yl]ethynyl]phenyl]-4-(1,2,4- triazol-1-yl)pyridine-2- carboxamide |
| 52 | 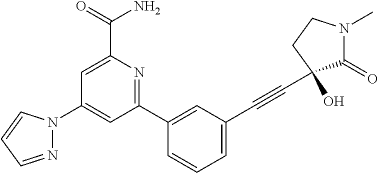 [SMILES: CN1CC[C@@](O)(C#Cc2cccc(-c3cc(-n4cccn4)cc(C(N)=O)n3)c2)C1=O] | 6-[3-[2-[(3R)-3-hydroxy-1- methyl-2-oxo-pyrrolidin-3- yl]ethynyl]phenyl]-4-pyrazol- 1-yl-pyridine-2-carboxamide |
| 53 | 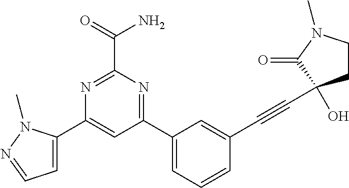 [SMILES: CN1CC[C@@](O)(C#Cc2cccc(-c3cc(-c4ccnn4C)nc(C(N)=O)n3)c2)C1=O] | 4-[3-[2-[(3R)-3-hydroxy-1- methyl-2-oxo-pyrrolidin-3- yl]ethynyl]phenyl]-6-(2- methylpyrazol-3-yl) pyrimidine-2-carboxamide |
| 54 | 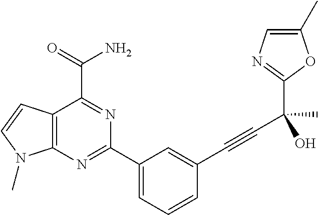 [SMILES: Cc1cnc([C@@](C)(O)C#Cc2cccc(-c3nc(C(N)=O)c4ccn(C)c4n3)c2)o1] | 2-[3-[(3S)-3-hydroxy-3-(5- methyloxazol-2-yl)but-1- ynyl]phenyl]-7-methyl- pyrrolo[2,3-d] pyrimidine-4-carboxamide |
| 55 | 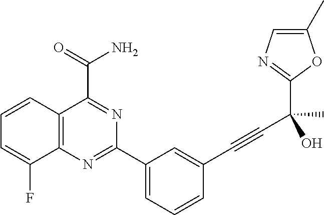 [SMILES: Cc1cnc([C@@](C)(O)C#Cc2cccc(-c3nc(C(N)=O)c4cccc(F)c4n3)c2)o1] | 8-fluoro-2-[3-[(3S)-3-hydroxy-3- (5-methyloxazol-2-yl)but-1-ynyl] phenyl]quinazoline-4- carboxamide |
| 56 | 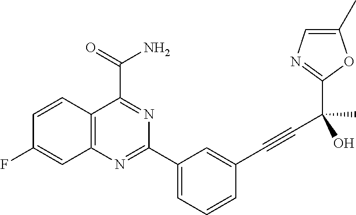 [SMILES: Cc1cnc([C@@](C)(O)C#Cc2cccc(-c3nc(C(N)=O)c4ccc(F)cc4n3)c2)o1] | 7-fluoro-2-[3-[(3S)-3-hydroxy- 3-(5-methyloxazol-2-yl)but-1- ynyl]phenyl]quinazoline-4- carboxamide |
| 57 | 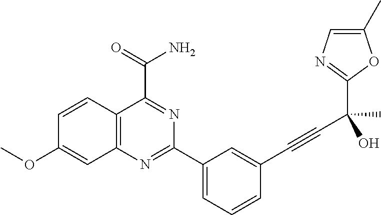 | 2-[3-[(3S)-3-hydroxy-3-(5- methyloxazol-2-yl)but-1- ynyl]phenyl]-7-methoxy- quinazoline-4-carboxamide |
| 58 | 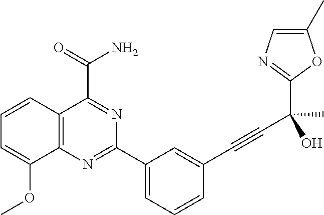 [SMILES: COc1cccc2c(C(N)=O)nc(-c3cccc(C#C[C@](C)(O)c4ncc(C)o4)c3)nc12] | 2-[3-[(3S)-3-hydroxy-3-(5- methyloxazol-2-yl)but-1- ynyl]phenyl]-8-methoxy- quinazoline-4-carboxamide |
| 59 | 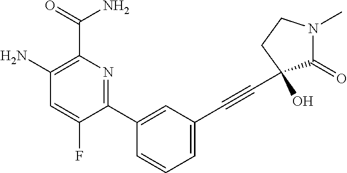 [SMILES: CN1CC[C@@](O)(C#Cc2cccc(-c3nc(C(N)=O)c(N)cc3F)c2)C1=O] | 3-amino-5-fluoro-6-[3-[2-[(3R)- 3-hydroxy-1-methyl-2-oxo- pyrrolidin-3-yl]ethynyl]phenyl] pyridine-2-carboxamide |
| 60 | 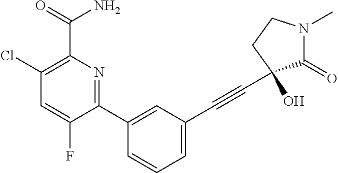 [SMILES: CN1CC[C@@](O)(C#Cc2cccc(-c3nc(C(N)=O)c(Cl)cc3F)c2)C1=O] | 3-chloro-5-fluoro-6-[3-[2-[(3R)- 3-hydroxy-1-methyl-2-oxo- pyrrolidin-3-yl]ethynyl]phenyl] pyridine-2-carboxamide |
| 61 | 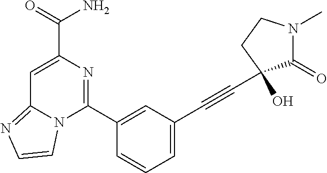 [SMILES: CN1CC[C@@](O)(C#Cc2cccc(-c3nc(C(N)=O)cc4nccn34)c2)C1=O] | 5-[3-[2-[(3R)-3-hydroxy-1- methyl-2-oxo-pyrrolidin-3- yl]ethynyl]phenyl]imidazo [1,2-c]pyrimidine-7- carboxamide |
| 62 | 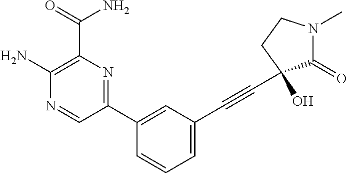 [SMILES: CN1CC[C@@](O)(C#Cc2cccc(-c3cnc(N)c(C(N)=O)n3)c2)C1=O] | 3-amino-6-[3-[2-[(3R)-3- hydroxy-1-methyl-2-oxo- pyrrolidin-3-yl]ethynyl]phenyl] pyrazine-2-carboxamide |
| 63 | 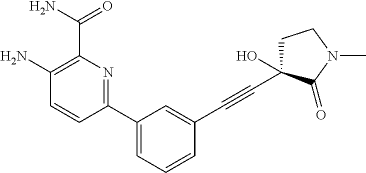 [SMILES: CN1CC[C@@](O)(C#Cc2cccc(-c3ccc(N)c(C(N)=O)n3)c2)C1=O] | 3-amino-6-[3-[2-[(3R)-3- hydroxy-1-methyl-2-oxo- pyrrolidin-3-yl]ethynyl] phenyl]pyridine-2- carboxamide |
| 64 | 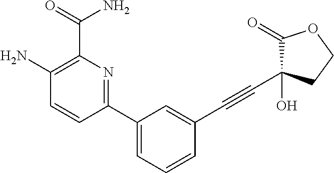 [SMILES: NC(=O)c1nc(-c2cccc(C#C[C@]3(O)CCOC3=O)c2)ccc1N] | 3-amino-6-[3-[2-[(3R)-3- hydroxy-2-oxo- tetrahydrofuran-3-yl] ethynyl]phenyl]pyridine- 2-carboxamide |
| 65 | 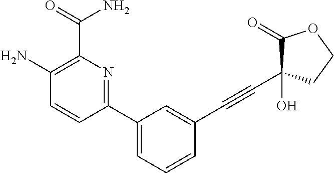 [SMILES: NC(=O)c1nc(-c2cccc(C#C[C@@]3(O)CCOC3=O)c2)ccc1N] | 3-amino-6-[3-[2-[(3S)-3- hydroxy-2-oxo- tetrahydrofuran-3-yl] ethynyl]phenyl]pyridine- 2-carboxamide |
| 66 | 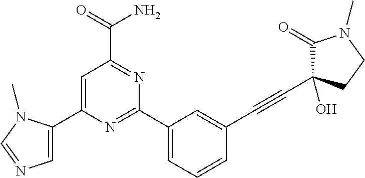 [SMILES: CN1CC[C@@](O)(C#Cc2cccc(-c3nc(C(N)=O)cc(-c4cncn4C)n3)c2)C1=O] | 2-[3-[2-[(3R)-3-hydroxy-1- methyl-2-oxo-pyrrolidin-3- yl]ethynyl]phenyl]-6-(3- methylimidazol-4-yl) pyrimidine-4-carboxamide |
| 67 | 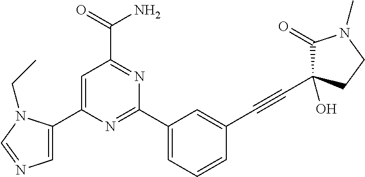 [SMILES: CCn1cncc1-c1cc(C(N)=O)nc(-c2cccc(C#C[C@]3(O)CCN(C)C3=O)c2)n1] | 6-(2-ethylpyrazol-3-yl)-2- [3-[2-[(3R)-3-hydroxy-1- methyl-2-oxo-pyrrolidin- 3-yl]ethynyl]phenyl] pyrimidine-4-carboxamide |
| 68 | 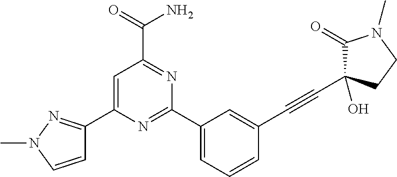 [SMILES: CN1CC[C@@](O)(C#Cc2cccc(-c3nc(C(N)=O)cc(-c4ccn(C)n4)n3)c2)C1=O] | 2-[3-[2-[(3R)-3-hydroxy-1- methyl-2-oxo-pyrrolidin-3- yl]ethynyl]phenyl]-6-(1- methylpyrazol-3-yl) pyrimidine-4-carboxamide |
| 69 | 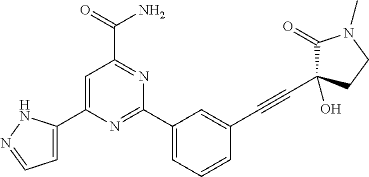 [SMILES: CN1CC[C@@](O)(C#Cc2cccc(-c3nc(C(N)=O)cc(-c4ccn[nH]4)n3)c2)C1=O] | 2-[3-[2-[(3R)-3-hydroxy-1- methyl-2-oxo-pyrrolidin-3- yl]ethynyl]phenyl]-6-(1H- pyrazol-5-yl)pyrimidine- 4-carboxamide |
| 70 | 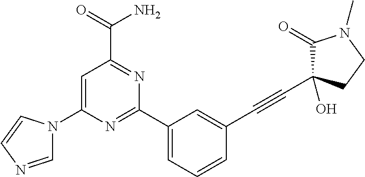 [SMILES: CN1CC[C@@](O)(C#Cc2cccc(-c3nc(C(N)=O)cc(-n4ccnc4)n3)c2)C1=O] | 2-[3-[2-[(3R)-3-hydroxy-1- methyl-2-oxo-pyrrolidin- 3-yl]ethynyl]phenyl]-6- imidazol-1-yl-pyrimidine- 4-carboxamide |
| 71 | 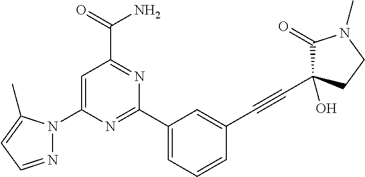 [SMILES: Cc1ccnn1-c1cc(C(N)=O)nc(-c2cccc(C#C[C@]3(O)CCN(C)C3=O)c2)n1] | 2-[3-[2-[(3R)-3-hydroxy-1- methyl-2-oxo-pyrrolidin-3- yl]ethynyl]phenyl]-6-(5- methylpyrazol-1-yl) pyrimidine-4-carboxamide |
| 72 | 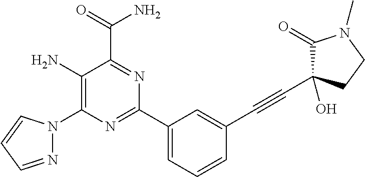 [SMILES: CN1CC[C@@](O)(C#Cc2cccc(-c3nc(C(N)=O)c(N)c(-n4cccn4)n3)c2)C1=O] | 5-amino-2-[3-[2-[(3R)-3- hydroxy-1-methyl-2-oxo- pyrrolidin-3-yl]ethynyl] phenyl]-6-pyrazol-1-yl- pyrimidine-4-carboxamide |
| 73 | 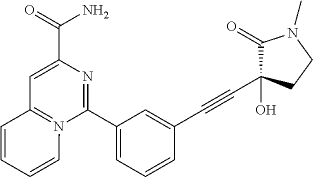 | 8-[3-[2-[(3R)-3-hydroxy-1- methyl-2-oxo-pyrrolidin-3- yl]ethynyl]phenyl]-1,7- naphthyridine-6-carboxamide |
| 74 | 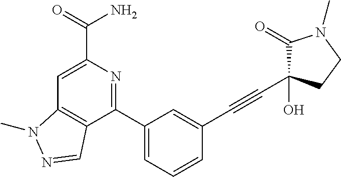 [SMILES: CN1CC[C@@](O)(C#Cc2cccc(-c3nc(C(N)=O)cc4c3cnn4C)c2)C1=O] | 4-[3-[2-[(3R)-3-hydroxy-1- methyl-2-oxo-pyrrolidin-3- yl]ethynyl]phenyl]-1-methyl- pyrazolo[4,3-c]pyridine-6- carboxamide |
| 75 | 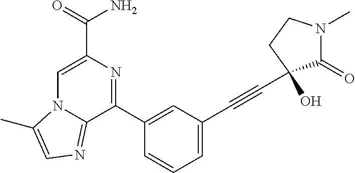 | 8-[3-[2-[(3R)-3-hydroxy-1- methyl-2-oxo-pyrrolidin-3- yl]ethynyl]phenyl]-3-methyl- imidazo[1,2-a]pyrazine-6- carboxamide |
| 76 | 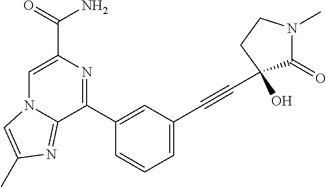 | 8-[3-[2-[(3R)-3-hydroxy-1- methyl-2-oxo-pyrrolidin-3- yl]ethynyl]phenyl]-2- methyl-imidazo[1,2-a] pyrazine-6-carboxamide |
| 77 | 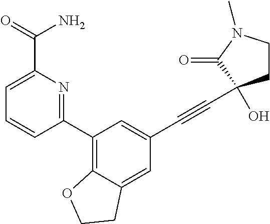 [SMILES: CN1CC[C@@](O)(C#Cc2cc3c(c(-c4cccc(C(N)=O)n4)c2)OCC3)C1=O] | 6-[5-[2-[(3R)-3-hydroxy-1- methyl-2-oxo-pyrrolidin-3- yl]ethynyl]-2,3- dihydrobenzofuran-7-yl] pyridine-2-carboxamide |
| 78 | 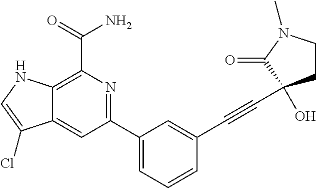 [SMILES: CN1CC[C@@](O)(C#Cc2cccc(-c3cc4c(Cl)c[nH]c4c(C(N)=O)n3)c2)C1=O] | 3-chloro-5-[3-[2-[(3R)-3- hydroxy-1-methyl-2-oxo- pyrrolidin-3-yl]ethynyl] phenyl]-1H-pyrrolo[2,3- c]pyridine-7-carboxamide |
| 79 | 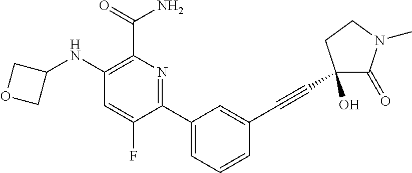 [SMILES: CN1CC[C@@](O)(C#Cc2cccc(-c3nc(C(N)=O)c(NC4COC4)cc3F)c2)C1=O] | 5-fluoro-6-[3-[2-[(3R)-3- hydroxy-1-methyl-2-oxo- pyrrolidin-3-yl]ethynyl] phenyl]-3-(oxetan-3- ylamino)pyridine-2- carboxamide |
| 80 | 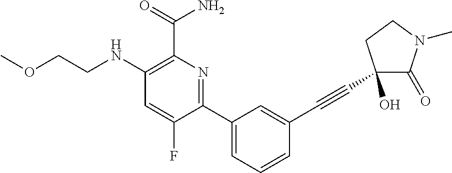 | 5-fluoro-6-[3-[2-[(3R)-3- hydroxy-1-methyl-2-oxo- pyrrolidin-3-yl]ethynyl] phenyl]-3-(2- methoxyethylamino) pyridine-2-carboxamide |
| 81 | 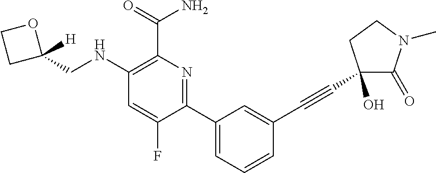 [SMILES: CN1CC[C@@](O)(C#Cc2cccc(-c3nc(C(N)=O)c(NC[C@@H]4CCO4)cc3F)c2)C1=O] | 5-fluoro-6-[3-[2-[(3R)-3- hydroxy-1-methyl-2-oxo- pyrrolidin-3-yl]ethynyl] phenyl]-3-[[(2S)-oxetan- 2-yl]methylamino] pyridine-2-carboxamide |
| 82 | 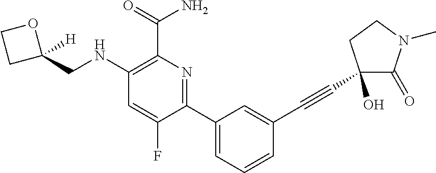 [SMILES: CN1CC[C@@](O)(C#Cc2cccc(-c3nc(C(N)=O)c(NC[C@H]4CCO4)cc3F)c2)C1=O] | 5-fluoro-6-[3-[2-[(3R)-3- hydroxy-1-methyl-2-oxo- pyrrolidin-3-yl]ethynyl] phenyl]-3-[[(2R)-oxetan- 2-yl]methylamino] pyridine-2-carboxamide |
| 83 | 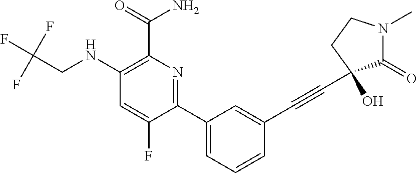 [SMILES: CN1CC[C@@](O)(C#Cc2cccc(-c3nc(C(N)=O)c(NCC(F)(F)F)cc3F)c2)C1=O] | 5-fluoro-6-[3-[2-[(3R)-3- hydroxy-1-methyl-2-oxo- pyrrolidin-3-yl]ethynyl] phenyl]-3-(2,2,2- trifluoroethylamino) pyridine-2-carboxamide |
| 84 | 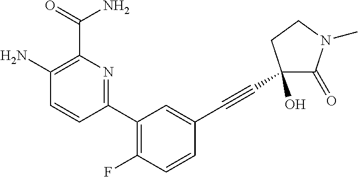 [SMILES: CN1CC[C@@](O)(C#Cc2ccc(F)c(-c3ccc(N)c(C(N)=O)n3)c2)C1=O] | 3-amino-6-[2-fluoro-5-[2-[(3R)- 3-hydroxy-1-methyl-2-oxo- pyrrolidin-3-yl]ethynyl]phenyl] pyridine-2-carboxamide |
| 85 | 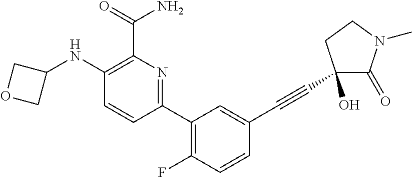 [SMILES: CN1CC[C@@](O)(C#Cc2ccc(F)c(-c3ccc(NC4COC4)c(C(N)=O)n3)c2)C1=O] | 6-[2-fluoro-5-[2-[(3R)-3- hydroxy-1-methyl-2-oxo- pyrrolidin-3-yl]ethynyl] phenyl]-3-(oxetan-3- ylamino)pyridine-2- carboxamide |
| 86 | 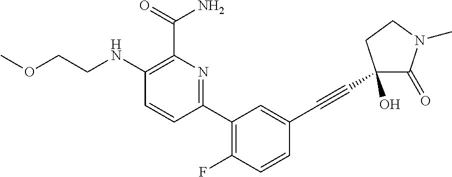 | 6-[2-fluoro-5-[2-[(3R)-3- hydroxy-1-methyl-2-oxo- pyrrolidin-3-yl]ethynyl] phenyl]-3-(2- methoxyethylamino) pyridine-2-carboxamide |
| 87 | 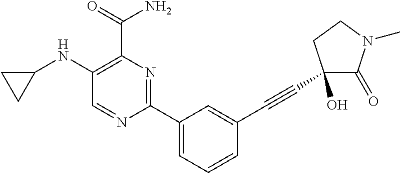 [SMILES: CN1CC[C@@](O)(C#Cc2cccc(-c3ncc(NC4CC4)c(C(N)=O)n3)c2)C1=O] | 5-(cyclopropylamino)-2-[3- [2-[(3R)-3-hydroxy-1-methyl- 2-oxo-pyrrolidin-3- yl]ethynyl]phenyl] pyrimidine-4-carboxamide |
| 88 | 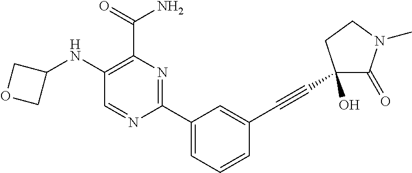 [SMILES: CN1CC[C@@](O)(C#Cc2cccc(-c3ncc(NC4COC4)c(C(N)=O)n3)c2)C1=O] | 2-[3-[2-[(3R)-3-hydroxy-1- methyl-2-oxo-pyrrolidin-3- yl]ethynyl]phenyl]-5- (oxetan-3-ylamino) pyrimidine-4-carboxamide |
| 89 | 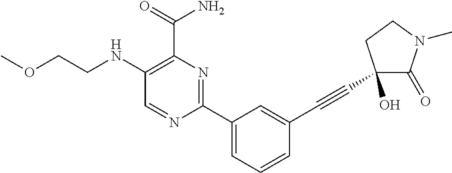 | 2-[3-[2-[(3R)-3-hydroxy-1- methyl-2-oxo-pyrrolidin-3- yl]ethynyl]phenyl]-5-(2- methoxyethylamino) pyrimidine-4-carboxamide |
| 90 | 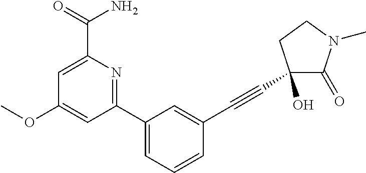 | 6-[3-[2-[(3R)-3-hydroxy-1- methyl-2-oxo-pyrrolidin-3- yl]ethynyl]phenyl]-4- methoxy-pyridine-2- carboxamide |
| 91 | 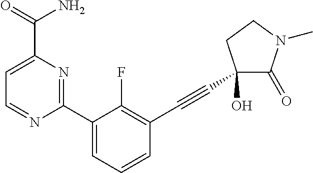 [SMILES: CN1CC[C@@](O)(C#Cc2cccc(-c3nccc(C(N)=O)n3)c2F)C1=O] | 2-[2-fluoro-3-[2-[(3R)-3- hydroxy-1-methyl-2-oxo- pyrrolidin-3-yl]ethynyl] phenyl]pyrimidine-4- carboxamide |
| 92 | 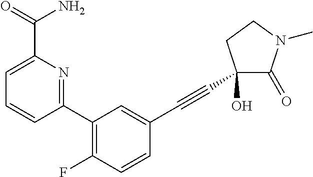 [SMILES: CN1CC[C@@](O)(C#Cc2ccc(F)c(-c3cccc(C(N)=O)n3)c2)C1=O] | 6-[2-fluoro-5-[2-[(3R)- 3-hydroxy-1-methyl-2- oxo-pyrrolidin-3-yl] ethynyl]phenyl]pyridine- 2-carboxamide |
| 93 | 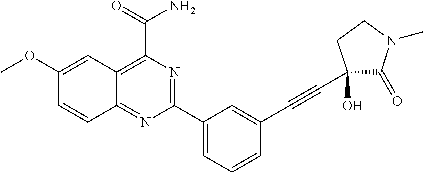 | 2-[3-[2-[(3R)-3-hydroxy-1- methyl-2-oxo-pyrrolidin-3- yl]ethynyl]phenyl]-6- methoxy-quinazoline- 4-carboxamide |
| 94 | 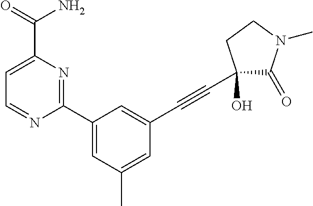 [SMILES: Cc1cc(C#C[C@]2(O)CCN(C)C2=O)cc(-c2nccc(C(N)=O)n2)c1] | 2-[3-[2-[(3R)-3-hydroxy-1- methyl-2-oxo-pyrrolidin-3- yl]ethynyl]-5-methyl- phenyl]pyrimidine-4- carboxamide |
| 95 | 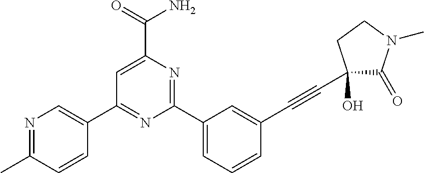 | 2-[3-[2-[(3R)-3-hydroxy-1- methyl-2-oxo-pyrrolidin-3- yl]ethynyl]phenyl]-6-(6- methyl-3-pyridyl)pyrimidine- 4-carboxamide |
| 96 | 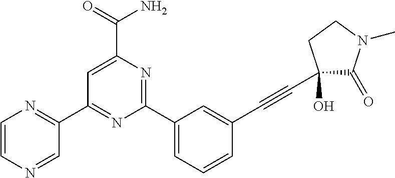 [SMILES: CN1CC[C@@](O)(C#Cc2cccc(-c3nc(C(N)=O)cc(-c4cnccn4)n3)c2)C1=O] | 2-[3-[2-[(3R)-3-hydroxy-1- methyl-2-oxo-pyrrolidin-3- yl]ethynyl]phenyl]-6- pyrazin-2-yl-pyrimidine- 4-carboxamide |
| 97 | 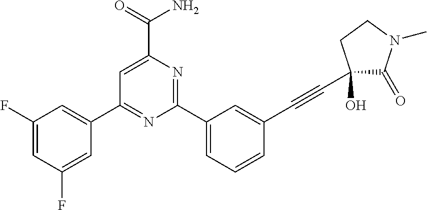 [SMILES: CN1CC[C@@](O)(C#Cc2cccc(-c3nc(C(N)=O)cc(-c4cc(F)cc(F)c4)n3)c2)C1=O] | 6-(3,5-difluorophenyl)-2-[3-[2- [(3R)-3-hydroxy-1-methyl-2- oxo-pyrrolidin-3-yl]ethynyl] phenyl]pyrimidine-4- carboxamide |
| 98 | 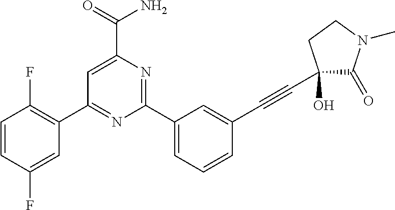 [SMILES: CN1CC[C@@](O)(C#Cc2cccc(-c3nc(C(N)=O)cc(-c4cc(F)ccc4F)n3)c2)C1=O] | 6-(2,5-difluorophenyl)-2-[3-[2- [(3R)-3-hydroxy-1-methyl-2- oxo-pyrrolidin-3-yl]ethynyl] phenyl]pyrimidine-4- carboxamide |
| 99 | 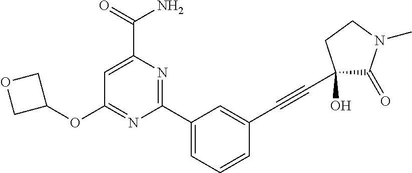 [SMILES: CN1CC[C@@](O)(C#Cc2cccc(-c3nc(OC4COC4)cc(C(N)=O)n3)c2)C1=O] | 2-[3-[2-[(3R)-3-hydroxy-1- methyl-2-oxo-pyrrolidin-3- yl]ethynyl]phenyl]-6- (oxetan-3-yloxy) pyrimidine-4-carboxamide |
| 100 | 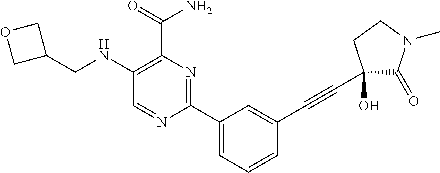 [SMILES: CN1CC[C@@](O)(C#Cc2cccc(-c3ncc(NCC4COC4)c(C(N)=O)n3)c2)C1=O] | 2-[3-[2-[(3R)-3-hydroxy-1- methyl-2-oxo-pyrrolidin-3- yl]ethynyl]phenyl]-5- (oxetan-3-ylmethylamino) pyrimidine-4-carboxamide |
| 101 | 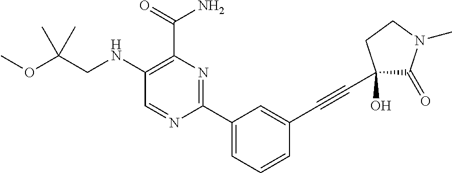 | 2-[3-[2-[(3R)-3-hydroxy-1- methyl-2-oxo-pyrrolidin-3- yl]ethynyl]phenyl]-5-[(2- methoxy-2-methyl- propyl)amino]pyrimidine- 4-carboxamide |
| 102 | 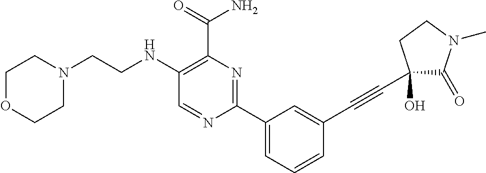 [SMILES: CN1CC[C@@](O)(C#Cc2cccc(-c3ncc(NCCN4CCOCC4)c(C(N)=O)n3)c2)C1=O] | 2-[3-[2-[(3R)-3-hydroxy-1- methyl-2-oxo-pyrrolidin-3- yl]ethynyl]phenyl]-5-(2- morpholinoethylamino) pyrimidine-4-carboxamide |
| 103 | 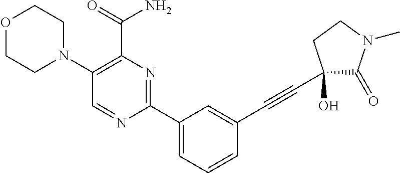 [SMILES: CN1CC[C@@](O)(C#Cc2cccc(-c3ncc(N4CCOCC4)c(C(N)=O)n3)c2)C1=O] | 2-[3-[2-[(3R)-3-hydroxy-1- methyl-2-oxo-pyrrolidin-3- yl]ethynyl]phenyl]-5- morpholino-pyrimidine- 4-carboxamide |
| 104 | 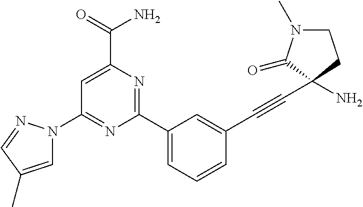 [SMILES: Cc1cnn(-c2cc(C(N)=O)nc(-c3cccc(C#C[C@]4(N)CCN(C)C4=O)c3)n2)c1] | 2-[3-[2-[(3R)-3-hydroxy-1- methyl-2-oxo-pyrrolidin-3- yl]ethynyl]phenyl]-6-(4- methylpyrazol-1-yl) pyrimidine-4-carboxamide |
| 105 | 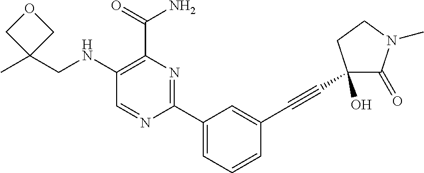 | 2-[3-[2-[(3R)-3-hydroxy-1- methyl-2-oxo-pyrrolidin-3- yl]ethynyl]phenyl]-5-[(3- methyloxetan-3-yl) methylamino]pyrimidine- 4-carboxamide |
| 106 | 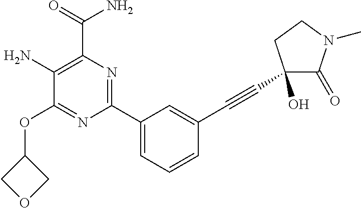 [SMILES: CN1CC[C@@](O)(C#Cc2cccc(-c3nc(OC4COC4)c(N)c(C(N)=O)n3)c2)C1=O] | 5-amino-2-[3-[2-[(3R)-3- hydroxy-1-methyl-2-oxo- pyrrolidin-3-yl]ethynyl] phenyl]-6-(oxetan-3- yloxy)pyrimidine-4- carboxamide |
| 107 | 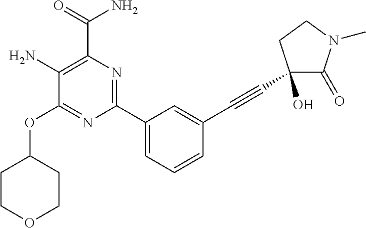 [SMILES: CN1CC[C@@](O)(C#Cc2cccc(-c3nc(OC4CCOCC4)c(N)c(C(N)=O)n3)c2)C1=O] | 5-amino-2-[3-[2-[(3R)-3- hydroxy-1-methyl-2-oxo- pyrrolidin-3-yl]ethynyl] phenyl]-6-tetrahydropyran- 4-yloxy-pyrimidine-4- carboxamide |
| 108 | 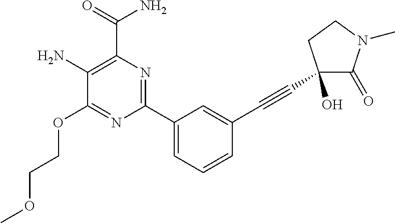 | 5-amino-2-[3-[2-[(3R)-3- hydroxy-1-methyl-2-oxo- pyrrolidin-3-yl]ethynyl] phenyl]-6-(2- methoxyethoxy)pyrimidine- 4-carboxamide |
| 109 | 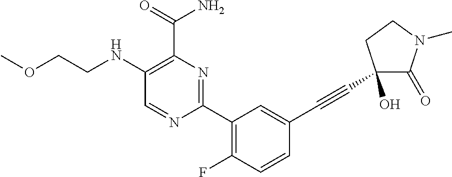 | 2-[2-fluoro-5-[2-[(3R)-3- hydroxy-1-methyl-2-oxo- pyrrolidin-3-yl]ethynyl] phenyl]-5-(2-methoxyethylamino) pyrimidine-4-carboxamide |
| 110 | 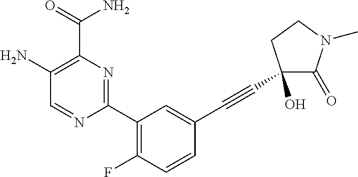 [SMILES: CN1CC[C@@](O)(C#Cc2ccc(F)c(-c3ncc(N)c(C(N)=O)n3)c2)C1=O] | 5-amino-2-[2-fluoro-5-[2-[(3R)- 3-hydroxy-1-methyl-2-oxo- pyrrolidin-3-yl]ethynyl] phenyl]pyrimidine-4-carboxamide |
| 111 | 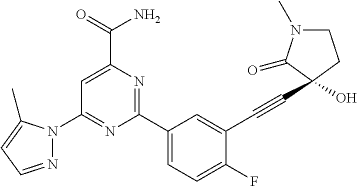 [SMILES: Cc1ccnn1-c1cc(C(N)=O)nc(-c2ccc(F)c(C#C[C@]3(O)CCN(C)C3=O)c2)n1] | 2-[4-fluoro-3-[2-[(3R)-3- hydroxy-1-methyl-2-oxo- pyrrolidin-3-yl]ethynyl] phenyl]-6-(5-methylpyrazol- 1-yl)pyrimidine-4- carboxamide |
| 112 | 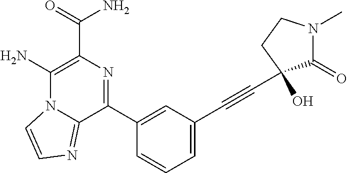 [SMILES: CN1CC[C@@](O)(C#Cc2cccc(-c3nc(C(N)=O)c(N)n4ccnc34)c2)C1=O] | 5-amino-8-[3-[2-[(3R)-3- hydroxy-1-methyl-2-oxo- pyrrolidin-3-yl]ethynyl] phenyl]imidazo[1,2-a] pyrazine-6-carboxamide |
| 113 | 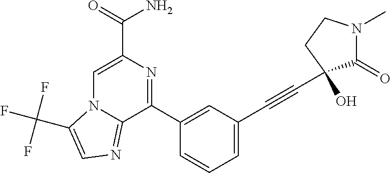 [SMILES: CN1CC[C@@](O)(C#Cc2cccc(-c3nc(C(N)=O)cn4c(C(F)(F)F)cnc34)c2)C1=O] | 8-[3-[2-[(3R)-3-hydroxy-1- methyl-2-oxo-pyrrolidin-3- yl]ethynyl]phenyl]-3- (trifluoromethyl)imidazo [1,2-a]pyrazine-6- carboxamide |
| 114 | 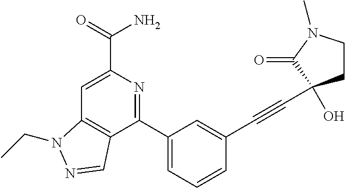 [SMILES: CCn1ncc2c(-c3cccc(C#C[C@]4(O)CCN(C)C4=O)c3)nc(C(N)=O)cc21] | 1-ethyl-4-[3-[2-[(3R)-3-hydroxy- 1-methyl-2-oxo-pyrrolidin-3-yl] ethynyl]phenyl]pyrazolo[4,3-c] pyridine-6-carboxamide |
| 115 | 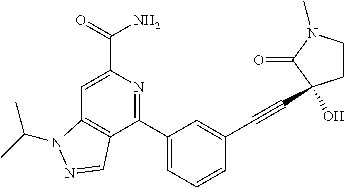 [SMILES: CC(C)n1ncc2c(-c3cccc(C#C[C@]4(O)CCN(C)C4=O)c3)nc(C(N)=O)cc21] | 4-[3-[2-[(3R)-3-hydroxy-1- methyl-2-oxo-pyrrolidin-3- yl]ethynyl]phenyl]-1- isopropyl-pyrazolo[4,3- c]pyridine-6-carboxamide |
| 116 | 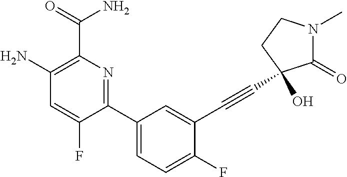 [SMILES: CN1CC[C@@](O)(C#Cc2cc(-c3nc(C(N)=O)c(N)cc3F)ccc2F)C1=O] | 3-amino-5-fluoro-6-[4-fluoro-3- [2-[(3R)-3-hydroxy-1-methyl-2- oxo-pyrrolidin-3-yl]ethynyl] phenyl]pyridine-2-carboxamide |
| 117 | 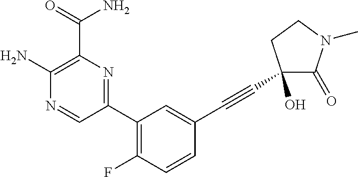 [SMILES: CN1CC[C@@](O)(C#Cc2ccc(F)c(-c3cnc(N)c(C(N)=O)n3)c2)C1=O] | 3-amino-6-[2-fluoro-5-[2-[(3R)- 3-hydroxy-1-methyl-2-oxo- pyrrolidin-3-yl]ethynyl]phenyl] pyrazine-2-carboxamide |
| 118 | 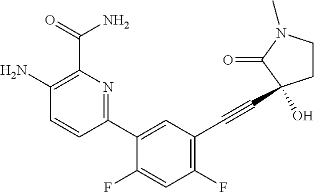 [SMILES: CN1CC[C@@](O)(C#Cc2cc(-c3ccc(N)c(C(N)=O)n3)c(F)cc2F)C1=O] | 3-amino-6-[2,4-difluoro-5-[2- [(3R)-3-hydroxy-1-methyl-2- oxo-pyrrolidin-3-yl]ethynyl] phenyl]pyridine-2-carboxamide |
| 119 | 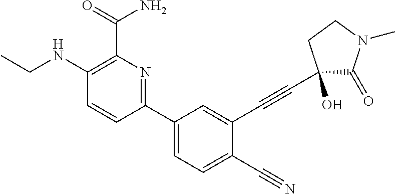 [SMILES: CCNc1ccc(-c2ccc(C#N)c(C#C[C@]3(O)CCN(C)C3=O)c2)nc1C(N)=O] | 6-[4-cyano-3-[2-[(3R)-3-hydroxy- 1-methyl-2-oxo-pyrrolidin-3-yl] ethynyl]phenyl]-3-(ethylamino) pyridine-2-carboxamide |
| 120 | 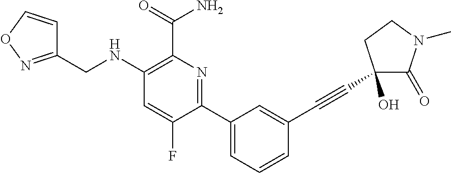 [SMILES: CN1CC[C@@](O)(C#Cc2cccc(-c3nc(C(N)=O)c(NCc4ccon4)cc3F)c2)C1=O] | 5-fluoro-6-[3-[2-[(3R)-3- hydroxy-1-methyl-2-oxo- pyrrolidin-3-yl]ethynyl] phenyl]-3-(isoxazol-3- ylmethylamino)pyridine- 2-carboxamide |
| 121 | 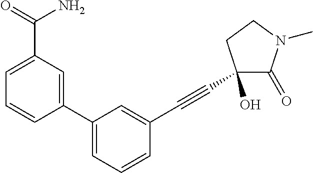 [SMILES: CN1CC[C@@](O)(C#Cc2cccc(-c3cccc(C(N)=O)c3)c2)C1=O] | 3-[3-[2-[(3R)-3-hydroxy-1- methyl-2-oxo-pyrrolidin-3- yl]ethynyl]phenyl]benzamide |
| 122 | 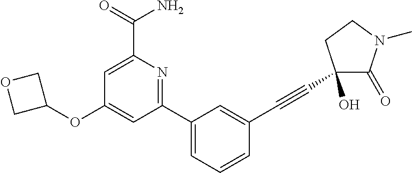 [SMILES: CN1CC[C@@](O)(C#Cc2cccc(-c3cc(OC4COC4)cc(C(N)=O)n3)c2)C1=O] | 6-[3-[2-[(3R)-3-hydroxy-1- methyl-2-oxo-pyrrolidin-3- yl]ethynyl]phenyl]-4- (oxetan-3-yloxy) pyridine-2-carboxamide |
| 123 | 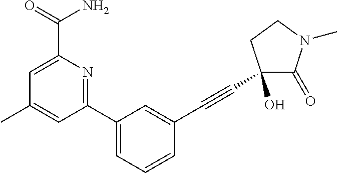 | 6-[3-[2-[(3R)-3-hydroxy-1- methyl-2-oxo-pyrrolidin-3- yl]ethynyl]phenyl]-4- methyl-pyridine-2- carboxamide |
| 124 | 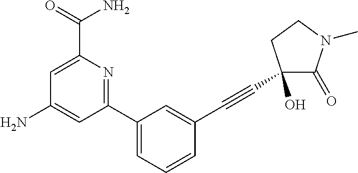 [SMILES: CN1CC[C@@](O)(C#Cc2cccc(-c3cc(N)cc(C(N)=O)n3)c2)C1=O] | 4-amino-6-[3-[2-[(3R)-3- hydroxy-1-methyl-2-oxo- pyrrolidin-3-yl]ethynyl] phenyl]pyridine-2- carboxamide |
| 125 | 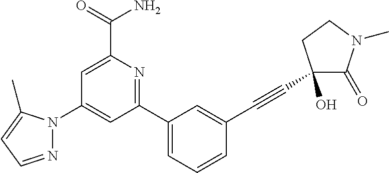 [SMILES: Cc1ccnn1-c1cc(C(N)=O)nc(-c2cccc(C#C[C@]3(O)CCN(C)C3=O)c2)c1] | 6-[3-[2-[(3R)-3-hydroxy- 1-methyl-2-oxo-pyrrolidin- 3-yl]ethynyl]phenyl]-4- (5-methylpyrazol-1-yl) pyridine-2-carboxamide |
| 126 | 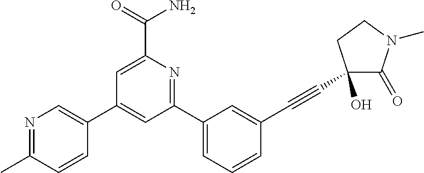 | 6-[3-[2-[(3R)-3-hydroxy-1- methyl-2-oxo-pyrrolidin-3- yl]ethynyl]phenyl]-4-(6- methyl-3-pyridyl)pyridine- 2-carboxamide |
| 127 | 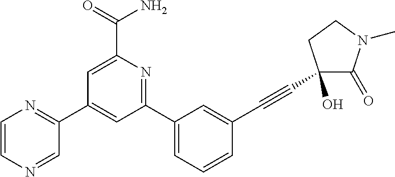 [SMILES: CN1CC[C@@](O)(C#Cc2cccc(-c3cc(-c4cnccn4)cc(C(N)=O)n3)c2)C1=O] | 6-[3-[2-[(3R)-3-hydroxy- 1-methyl-2-oxo- pyrrolidin-3-yl]ethynyl] phenyl]-4-pyrazin-2-yl- pyridine-2-carboxamide |
| 128 | 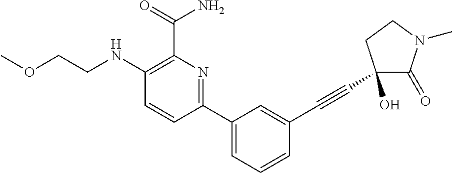 | 6-[3-[2-[(3R)-3-hydroxy-1- methyl-2-oxo-pyrrolidin-3- yl]ethynyl]phenyl]-3-(2- methoxyethylamino) pyridine-2-carboxamide |
| 129 | 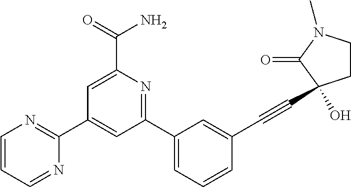 [SMILES: CN1CC[C@@](O)(C#Cc2cccc(-c3cc(-c4ncccn4)cc(C(N)=O)n3)c2)C1=O] | 6-[3-[2-[(3R)-3-hydroxy-1- methyl-2-oxo-pyrrolidin-3- yl]ethynyl]phenyl]-4- pyrimidin-2-yl-pyridine- 2-carboxamide |
| 130 | 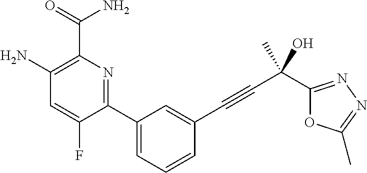 [SMILES: Cc1nnc([C@](C)(O)C#Cc2cccc(-c3nc(C(N)=O)c(N)cc3F)c2)o1] | 3-amino-5-fluoro-6-[3-[(3R)-3- hydroxy-3-(5-methyl-1,3,4- oxadiazol-2-yl)but-1-ynyl] phenyl]pyridine-2-carboxamide |
| 131 | 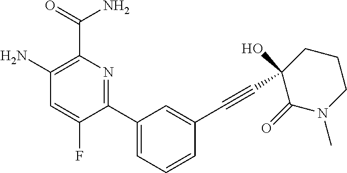 [SMILES: CN1CCC[C@@](O)(C#Cc2cccc(-c3nc(C(N)=O)c(N)cc3F)c2)C1=O] | 3-amino-5-fluoro-6-[3-[2- [(3R)-3-hydroxy-1-methyl- 2-oxo-3-piperidyl]ethynyl] phenyl]pyridine-2-carboxamide |
| 132 | 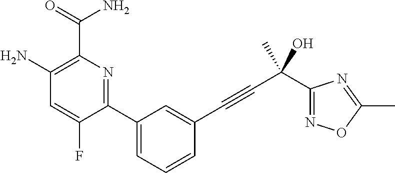 [SMILES: Cc1nc([C@](C)(O)C#Cc2cccc(-c3nc(C(N)=O)c(N)cc3F)c2)no1] | 3-amino-5-fluoro-6-[3-[(3R)-3- hydroxy-3-(5-methyl-1,2,4- oxadiazol-3-yl)but-1-ynyl] phenyl]pyridine-2-carboxamide |
| 133 | 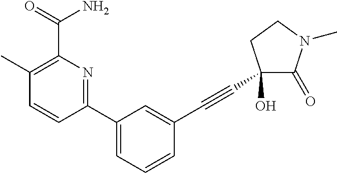 | 6-[3-[2-[(3R)-3-hydroxy-1- methyl-2-oxo-pyrrolidin- 3-yl]ethynyl]phenyl]-3- methyl-pyridine-2-carboxamide |
| 134 | 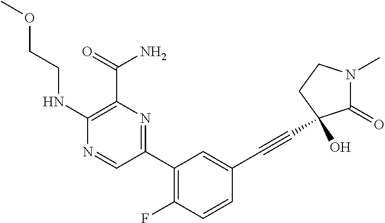 | 6-[2-fluoro-5-[2-[(3R)-3- hydroxy-1-methyl-2-oxo- pyrrolidin-3-yl]ethynyl] phenyl]-3-(2- methoxyethylamino) pyrazine-2-carboxamide |
| 135 | 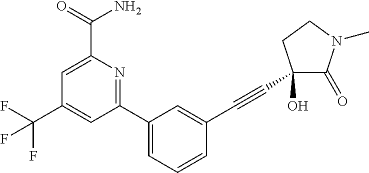 [SMILES: CN1CC[C@@](O)(C#Cc2cccc(-c3cc(C(F)(F)F)cc(C(N)=O)n3)c2)C1=O] | 6-[3-[2-[(3R)-3-hydroxy-1- methyl-2-oxo-pyrrolidin-3- yl]ethynyl]phenyl]-4- (trifluoromethyl) pyridine-2-carboxamide |
| 136 | 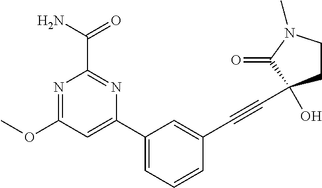 [SMILES: COc1cc(-c2cccc(C#C[C@]3(O)CCN(C)C3=O)c2)nc(C(N)=O)n1] | 4-[3-[2-[(3R)-3-hydroxy-1- methyl-2-oxo-pyrrolidin-3- yl]ethynyl]phenyl]-6- methoxy-pyrimidine-2- carboxamide |
| 137 | 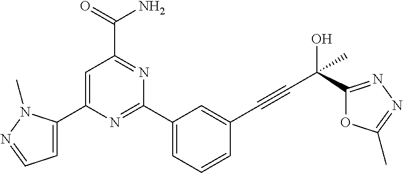 [SMILES: Cc1nnc([C@](C)(O)C#Cc2cccc(-c3nc(C(N)=O)cc(-c4ccnn4C)n3)c2)o1] | 2-[3-[(3R)-3-hydroxy-3-(5- methyl-1,3,4-oxadiazol-2-yl) but-1-ynyl]phenyl]-6-(2- methylpyrazol-3- yl)pyrimidine-4-carboxamide |
| 138 | 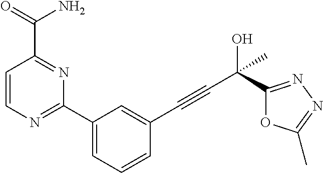 [SMILES: Cc1nnc([C@](C)(O)C#Cc2cccc(-c3nccc(C(N)=O)n3)c2)o1] | 2-[3-[(3R)-3-hydroxy-3-(5- methyl-1,3,4-oxadiazol-2-yl) but-1-ynyl]phenyl]pyrimidine- 4-carboxamide |
| 139 | 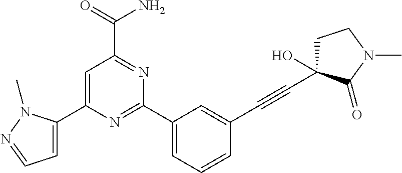 [SMILES: CN1CC[C@@](O)(C#Cc2cccc(-c3nc(C(N)=O)cc(-c4ccnn4C)n3)c2)C1=O] | 2-[3-[2-[(3R)-3-hydroxy-1- methyl-2-oxo-pyrrolidin-3- yl]ethynyl]phenyl]-6- pyrazol-1-yl-pyrimidine- 4-carboxamide |
| 140 | 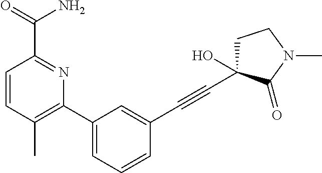 [SMILES: Cc1ccc(C(N)=O)nc1-c1cccc(C#C[C@]2(O)CCN(C)C2=O)c1] | 6-[3-[2-[(3R)-3-hydroxy-1- methyl-2-oxo-pyrrolidin-3- yl]ethynyl]phenyl]-5-methyl- pyridine-2-carboxamide |
| 141 | 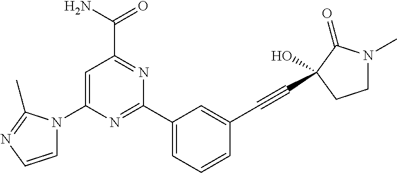 [SMILES: Cc1nccn1-c1cc(C(N)=O)nc(-c2cccc(C#C[C@]3(O)CCN(C)C3=O)c2)n1] | 2-[3-[2-[(3R)-3-hydroxy-1- methyl-2-oxo-pyrrolidin-3- yl]ethynyl]phenyl]-6-(2- methylimidazol-1-yl) pyrimidine-4-carboxamide |
| 142 | 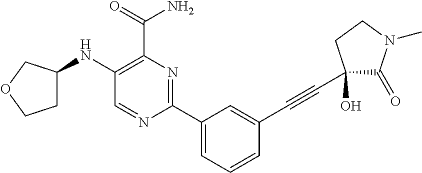 [SMILES: CN1CC[C@@](O)(C#Cc2cccc(-c3ncc(N[C@H]4CCOC4)c(C(N)=O)n3)c2)C1=O] | 2-[3-[2-[(3R)-3-hydroxy-1- methyl-2-oxo-pyrrolidin-3- yl]ethynyl]phenyl]-5-[[(3S)- tetrahydrofuran-3-yl]amino] pyrimidine-4-carboxamide |
| 143 | 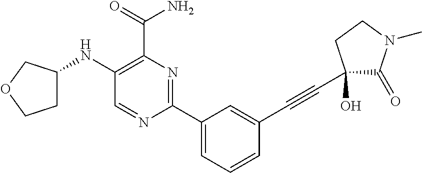 [SMILES: CN1CC[C@@](O)(C#Cc2cccc(-c3ncc(N[C@@H]4CCOC4)c(C(N)=O)n3)c2)C1=O] | 2-[3-[2-[(3R)-3-hydroxy-1- methyl-2-oxo-pyrrolidin-3- yl]ethynyl]phenyl]-5-[[(3R)- tetrahydrofuran-3-yl]amino] pyrimidine-4-carboxamide |
| 144 | 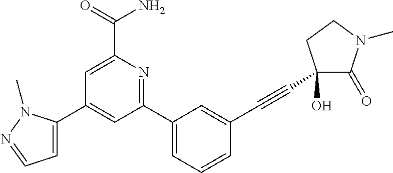 [SMILES: CN1CC[C@@](O)(C#Cc2cccc(-c3cc(-c4ccnn4C)cc(C(N)=O)n3)c2)C1=O] | 6-[3-[2-[(3R)-3-hydroxy-1- methyl-2-oxo-pyrrolidin-3- yl]ethynyl]phenyl]-4-(2- methylpyrazol-3-yl) pyridine-2-carboxamide |
| 145 | 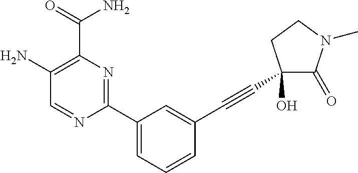 [SMILES: CN1CC[C@@](O)(C#Cc2cccc(-c3ncc(N)c(C(N)=O)n3)c2)C1=O] | 5-amino-2-[3-[2-[(3R)-3- hydroxy-1-methyl-2-oxo- pyrrolidin-3-yl]ethynyl] phenyl]pyrimidine-4- carboxamide |
| 146 | 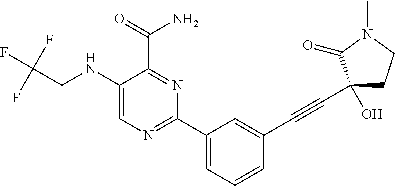 [SMILES: CN1CC[C@@](O)(C#Cc2cccc(-c3ncc(NCC(F)(F)F)c(C(N)=O)n3)c2)C1=O] | 2-[3-[2-[(3R)-3-hydroxy-1- methyl-2-oxo-pyrrolidin-3- yl]ethynyl]phenyl]-5-(2,2,2- trifluoroethylamino) pyrimidine-4-carboxamide |
| 147 | 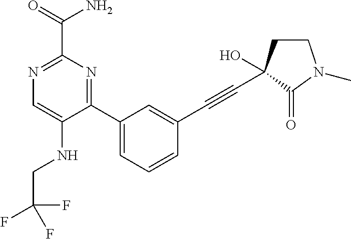 [SMILES: CN1CC[C@](O)(C#Cc2cccc(-c3nc(C(N)=O)ncc3NCC(F)(F)F)c2)C1=O] | 4-[3-[2-[(3R)-3-hydroxy-1- methyl-2-oxo-pyrrolidin- 3-yl]ethynyl]phenyl]-5- (2,2,2-trifluoroethylamino) pyrimidine-2-carboxamide |
| 148 | 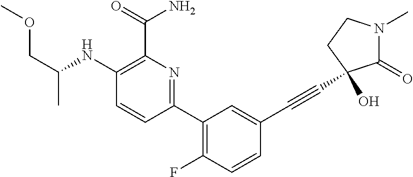 | 6-[2-fluoro-5-[2-[(3R)-3- hydroxy-1-methyl-2-oxo- pyrrolidin-3-yl]ethynyl] phenyl]-3-[[(1R)-2-methoxy- 1-methyl-ethyl]amino] pyridine-2-carboxamide |
| 149 | 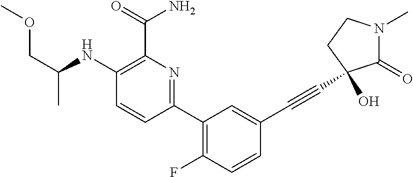 | 6-[2-fluoro-5-[2-[(3R)-3- hydroxy-1-methyl-2-oxo- pyrrolidin-3-yl]ethynyl] phenyl]-3-[[(1S)-2- methoxy-1-methyl-ethyl] amino]pyridine-2-carboxamide |
| 150 | 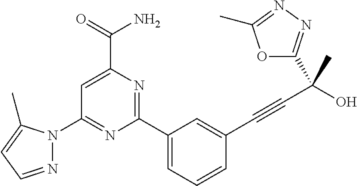 [SMILES: Cc1nnc([C@](C)(O)C#Cc2cccc(-c3nc(C(N)=O)cc(-n4nccc4C)n3)c2)o1] | 2-[3-[(3R)-3-hydroxy-3-(5- methyl-1,3,4-oxadiazol-2- yl)but-1-ynyl]phenyl]-6-(5- methylpyrazol-1- yl)pyrimidine-4-carboxamide |
| 151 | 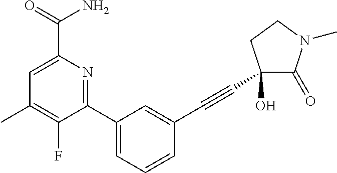 | 5-f1uoro-6-[3-[2-[(3R)-3- hydroxy-1-methyl-2-oxo- pyrrolidin-3-yl]ethynyl] phenyl]-4-methyl- pyridine-2-carboxamide |
| 152 | 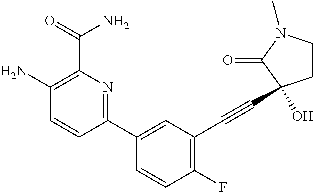 [SMILES: CN1CC[C@@](O)(C#Cc2cc(-c3ccc(N)c(C(N)=O)n3)ccc2F)C1=O] | 3-amino-6-[4-fluoro-3-[2-[(3R)- 3-hydroxy-1-methyl-2-oxo- pyrrolidin-3-yl]ethynyl]phenyl] pyridine-2-carboxamide |
| 153 | 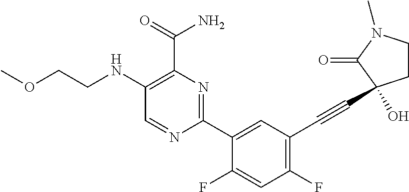 [SMILES: COCCNc1cnc(-c2cc(C#C[C@]3(O)CCN(C)C3=O)c(F)cc2F)nc1C(N)=O] | 2-[2,4-difluoro-5-[2-[(3R)-3- hydroxy-1-methyl-2-oxo- pyrrolidin-3-yl]ethynyl]phenyl]- 5-(2-methoxyethylamino) pyrimidine-4-carboxamide |
| 154 | 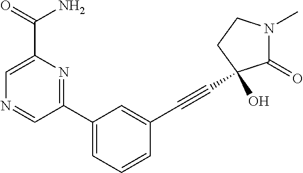 [SMILES: CN1CC[C@@](O)(C#Cc2cccc(-c3cncc(C(N)=O)n3)c2)C1=O] | 6-[3-[2-[(3R)-3-hydroxy-1- methyl-2-oxo-pyrrolidin-3- yl]ethynyl]phenyl]pyrazine- 2-carboxamide |
| 155 | 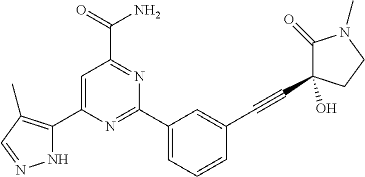 [SMILES: Cc1cn[nH]c1-c1cc(C(N)=O)nc(-c2cccc(C#C[C@]3(O)CCN(C)C3=O)c2)n1] | 2-[3-[2-[(3R)-3-hydroxy-1- methyl-2-oxo-pyrrolidin-3- yl]ethynyl]phenyl]-6-(4- methyl-1H-pyrazol- 5-yl)pyrimidine-4-carboxamide |
| 156 | 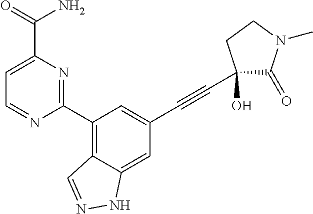 [SMILES: CN1CC[C@@](O)(C#Cc2cc(-c3nccc(C(N)=O)n3)c3cn[nH]c3c2)C1=O] | 2-[6-[2-[(3R)-3-hydroxy-1- methyl-2-oxo-pyrrolidin-3- yl]ethynyl]-1H-indazol- 4-yl]pyrimidine-4-carboxamide |
| 157 | 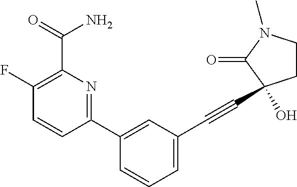 [SMILES: CN1CC[C@@](O)(C#Cc2cccc(-c3ccc(F)c(C(N)=O)n3)c2)C1=O] | 3-fluoro-6-[3-[2-[(3R)-3- hydroxy-1-methyl-2-oxo- pyrrolidin-3-yl]ethynyl]phenyl] pyridine-2-carboxamide |
| 158 | 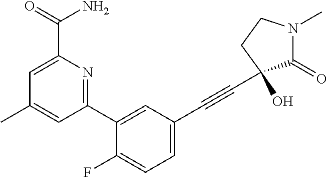 | 6-[2-fluoro-5-[2-[(3R)-3- hydroxy-1-methyl-2-oxo- pyrrolidin-3-yl]ethynyl] phenyl]-4-methyl- pyridine-2-carboxamide |
| 159 | 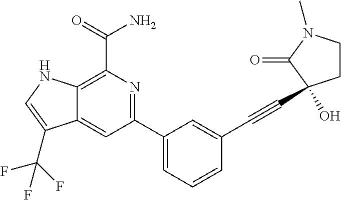 [SMILES: CN1CC[C@@](O)(C#Cc2cccc(-c3cc4c(C(F)(F)F)c[nH]c4c(C(N)=O)n3)c2)C1=O] | 5-[3-[2-[(3R)-3-hydroxy-1- methyl-2-oxo-pyrrolidin-3- yl]ethynyl]phenyl]-3- (trifluoromethyl)-1H- pyrrolo[2,3-c]pyridine- 7-carboxamide |
| 160 | 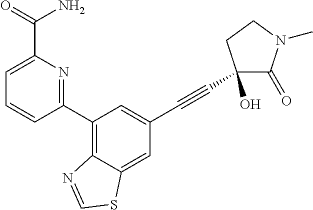 [SMILES: CN1CC[C@@](O)(C#Cc2cc(-c3cccc(C(N)=O)n3)c3ncsc3c2)C1=O] | 6-[6-[2-[(3R)-3-hydroxy-1- methyl-2-oxo-pyrrolidin-3- yl]ethynyl]-1,3-benzothiazol- 4-yl]pyridine-2-carboxamide |
| 161 | !! EMBED ChemDraw.Document. 6.0 | 8-[3-fluoro-5-[2-[(3R)-3- |
| | 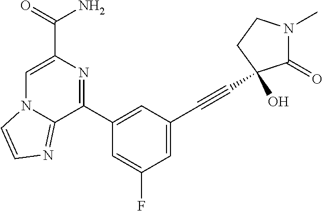 [SMILES: CN1CC[C@@](O)(C#Cc2cc(F)cc(-c3nc(C(N)=O)cn4ccnc34)c2)C1=O] | hydroxy-1-methyl-2-oxo- pyrrolidin-3-yl]ethynyl] phenyl]imidazo[1,2-a] pyrazine-6-carboxamide |
| 162 | 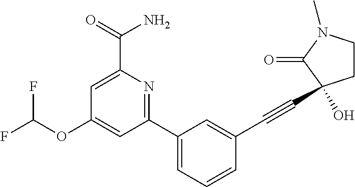 [SMILES: CN1CC[C@@](O)(C#Cc2cccc(-c3cc(OC(F)F)cc(C(N)=O)n3)c2)C1=O] | 4-(difluoromethoxy)-6-[3- [2-[(3R)-3-hydroxy-1- methyl-2-oxo-pyrrolidin-3- yl]ethynyl]phenyl]pyridine- 2-carboxamide |
| 163 | 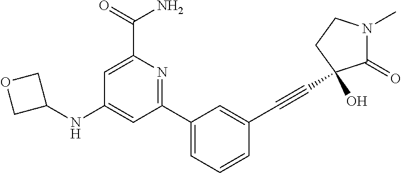 [SMILES: CN1CC[C@@](O)(C#Cc2cccc(-c3cc(NC4COC4)cc(C(N)=O)n3)c2)C1=O] | 6-[3-[2-[(3R)-3-hydroxy-1- methyl-2-oxo-pyrrolidin-3- yl]ethynyl]phenyl]-4- (oxetan-3-ylamino) pyridine-2-carboxamide |
| 164 | 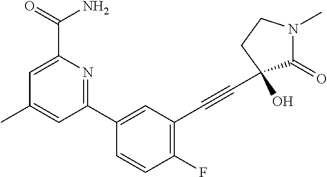 | 6-[4-fluoro-3-[2-[(3R)-3- hydroxy-1-methyl-2-oxo- pyrrolidin-3-yl]ethynyl] phenyl]-4-methyl- pyridine-2-carboxamide |
| 165 | 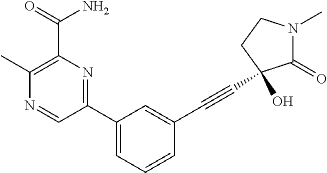 | 6-[3-[2-[(3R)-3-hydroxy-1- methyl-2-oxo-pyrrolidin-3- yl]ethynyl]phenyl]-3-methyl- pyrazine-2-carboxamide |
| 166 | 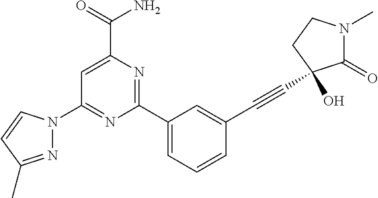 [SMILES: Cc1ccn(-c2cc(C(N)=O)nc(-c3cccc(C#C[C@]4(O)CCN(C)C4=O)c3)n2)n1] | 2-[3-[2-[(3R)-3-hydroxy-1- methyl-2-oxo-pyrrolidin-3- yl]ethynyl]phenyl]-6-(3- methylpyrazol-1-yl) pyrimidine-4-carboxamide |
| 167 | 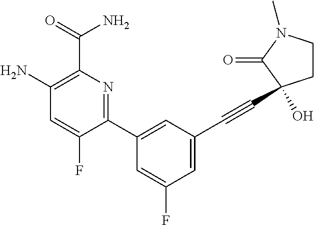 [SMILES: CN1CC[C@@](O)(C#Cc2cc(F)cc(-c3nc(C(N)=O)c(N)cc3F)c2)C1=O] | 3-amino-5-fluoro-6-[3-fluoro-5- [2-[(3R)-3-hydroxy-1-methyl-2- oxo-pyrrolidin-3-yl]ethynyl] phenyl]pyridine-2-carboxamide |
| 168 | 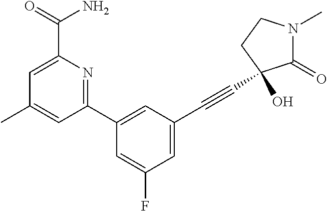 | 6-[3-fluoro-5-[2-[(3R)-3- hydroxy-1-methyl-2-oxo- pyrrolidin-3-yl]ethynyl] phenyl]-4-methyl- pyridine-2-carboxamide |
| 169 | 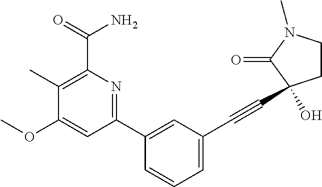 [SMILES: COc1cc(-c2cccc(C#C[C@]3(O)CCN(C)C3=O)c2)nc(C(N)=O)c1C] | 6-[3-[2-[(3R)-3-hydroxy-1- methyl-2-oxo-pyrrolidin-3- yl]ethynyl]phenyl]-4- methoxy-3-methyl-pyridine- 2-carboxamide |
| 170 | 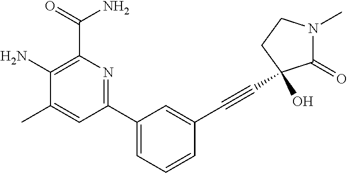 | 3-amino-6-[3-[2-[(3R)-3- hydroxy-1-methyl-2-oxo- pyrrolidin-3-yl]ethynyl] phenyl]-4-methyl- pyridine-2-carboxamide |
| 171 | 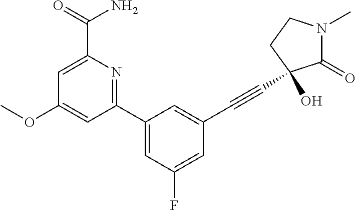 | 6-[3-fluoro-5-[2-[(3R)-3- hydroxy-1-methyl-2-oxo- pyrrolidin-3-yl]ethynyl] phenyl]-4-methoxy- pyridine-2-carboxamide |
| 172 | 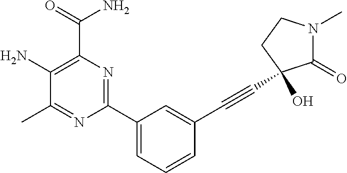 | 5-amino-2-[3-[2-[(3R)-3- hydroxy-1-methyl-2-oxo- pyrrolidin-3-yl]ethynyl] phenyl]-6-methyl- pyrimidine-4-carboxamide |
| 173 | 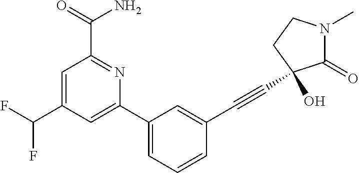 [SMILES: CN1CC[C@@](O)(C#Cc2cccc(-c3cc(C(F)F)cc(C(N)=O)n3)c2)C1=O] | 4-(difluoromethyl)-6-[3-[2- [(3R)-3-hydroxy-1-methyl- 2-oxo-pyrrolidin-3-yl] ethynyl]phenyl]pyridine-2- carboxamide |
| 174 | 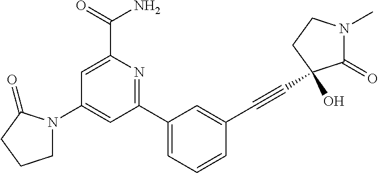 [SMILES: CN1CC[C@@](O)(C#Cc2cccc(-c3cc(N4CCCC4=O)cc(C(N)=O)n3)c2)C1=O] | 6-[3-[2-[(3R)-3-hydroxy-1- methyl-2-oxo-pyrrolidin-3- yl]ethynyl]phenyl]-4-(2- oxopyrrolidin-1-yl) pyridine-2-carboxamide |
| 175 | 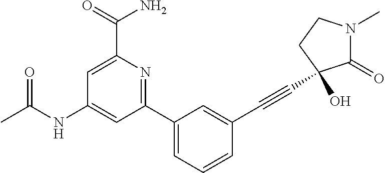 [SMILES: CC(=O)Nc1cc(C(N)=O)nc(-c2cccc(C#C[C@]3(O)CCN(C)C3=O)c2)c1] | 4-acetamido-6-[3-[2-[(3R)-3- hydroxy-1-methyl-2-oxo- pyrrolidin-3-yl]ethynyl]phenyl] pyridine-2-carboxamide |
| 176 | 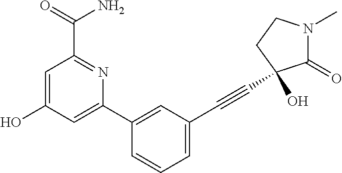 [SMILES: CN1CC[C@@](O)(C#Cc2cccc(-c3cc(O)cc(C(N)=O)n3)c2)C1=O] | 4-hydroxy-6-[3-[2-[(3R)-3- hydroxy-1-methyl-2-oxo- pyrrolidin-3-yl]ethynyl] phenyl]pyridine-2-carboxamide |
| 177 | 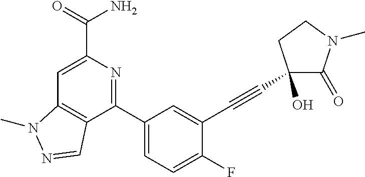 | 4-[4-fluoro-3-[2-[(3R)-3- hydroxy-1-methyl-2-oxo- pyrrolidin-3-yl]ethynyl] phenyl]-1-methyl-pyrazolo [4,3-c]pyridine-6-carboxamide |
| 178 | 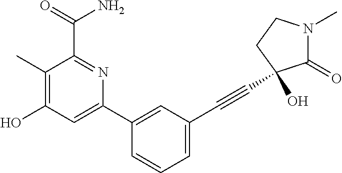 [SMILES: Cc1c(O)cc(-c2cccc(C#C[C@]3(O)CCN(C)C3=O)c2)nc1C(N)=O] | 4-hydroxy-6-[3-[2-[(3R)-3- hydroxy-1-methyl-2-oxo- pyrrolidin-3-yl]ethynyl] phenyl]-3-methyl-pyridine- 2-carboxamide |
| 179 | 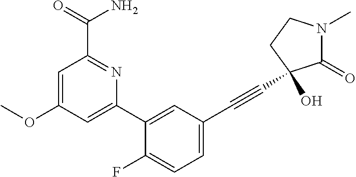 | 6-[2-fluoro-5-[2-[(3R)-3- hydroxy-1-methyl-2-oxo- pyrrolidin-3-yl]ethynyl] phenyl]-4-methoxy- pyridine-2-carboxamide |
| 180 | 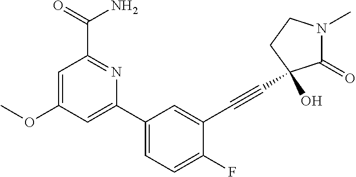 | 6-[4-fluoro-3-[2-[(3R)-3- hydroxy-1-methyl-2-oxo- pyrrolidin-3-yl]ethynyl] phenyl]-4-methoxy- pyridine-2-carboxamide |
| 181 | 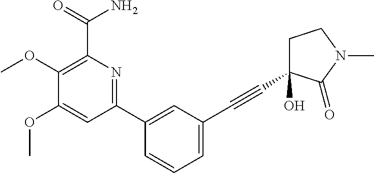 | 6-[3-[2-[(3R)-3-hydroxy- 1-methyl-2-oxo-pyrrolidin- 3-yl]ethynyl]phenyl]-3,4- dimethoxy-pyridine- 2-carboxamide |
| 182 | 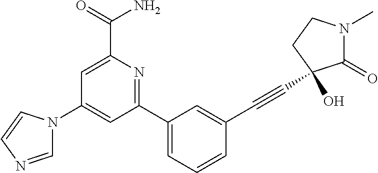 [SMILES: CN1CC[C@@](O)(C#Cc2cccc(-c3cc(-n4ccnc4)cc(C(N)=O)n3)c2)C1=O] | 6-[3-[2-[(3R)-3-hydroxy- 1-methyl-2-oxo-pyrrolidin- 3-yl]ethynyl]phenyl]-4- imidazol-1-yl-pyridine- 2-carboxamide |
| 183 | 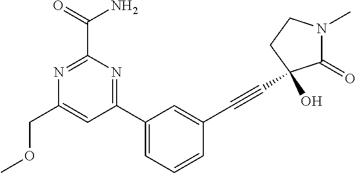 | 4-[3-[2-[(3R)-3-hydroxy- 1-methyl-2-oxo-pyrrolidin- 3-yl]ethynyl]phenyl]-6- (methoxymethyl)pyrimidine- 2-carboxamide |
| 184 | 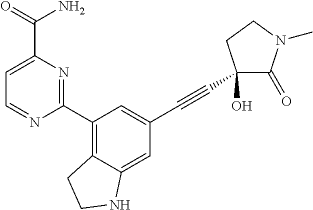 [SMILES: CN1CC[C@@](O)(C#Cc2cc3c(c(-c4nccc(C(N)=O)n4)c2)CCN3)C1=O] | 2-[6-[2-[(3R)-3-hydroxy-1- methyl-2-oxo-pyrrolidin-3- yl]ethynyl]indolin-4-yl] pyrimidine-4-carboxamide |
| 185 | 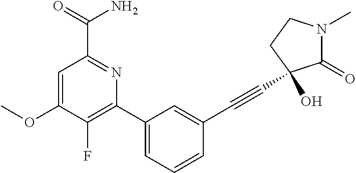 | 5-fluoro-6-[3-[2-[(3R)-3- hydroxy-1-methyl-2-oxo- pyrrolidin-3-yl]ethynyl] phenyl]-4-methoxy- pyridine-2-carboxamide |
| 186 | 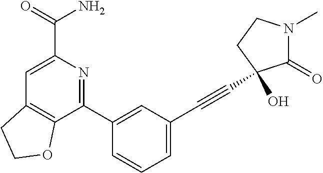 [SMILES: CN1CC[C@@](O)(C#Cc2cccc(-c3nc(C(N)=O)cc4c3OCC4)c2)C1=O] | 7-[3-[2-[(3R)-3-hydroxy- 1-methyl-2-oxo-pyrrolidin- 3-yl]ethynyl]phenyl]-2,3- dihydrofuro[2,3-c]pyridine- 5-carboxamide |
| 187 | 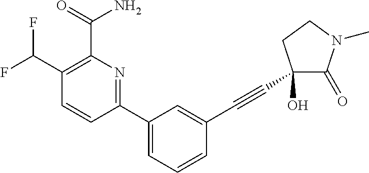 [SMILES: CN1CC[C@@](O)(C#Cc2cccc(-c3ccc(C(F)F)c(C(N)=O)n3)c2)C1=O] | 3-(difluoromethyl)-6-[3-[2- [(3R)-3-hydroxy-1-methyl- 2-oxo-pyrrolidin-3-yl] ethynyl]phenyl]pyridine- 2-carboxamide |
| 188 | 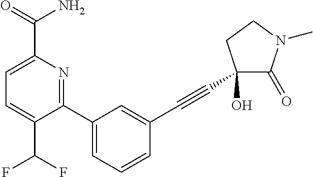 [SMILES: CN1CC[C@@](O)(C#Cc2cccc(-c3nc(C(N)=O)ccc3C(F)F)c2)C1=O] | 5-(difluoromethyl)-6-[3-[2- [(3R)-3-hydroxy-1-methyl- 2-oxo-pyrrolidin-3- yl]ethynyl]phenyl]pyridine- 2-carboxamide |
| 189 | 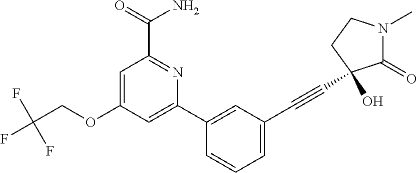 [SMILES: CN1CC[C@@](O)(C#Cc2cccc(-c3cc(OCC(F)(F)F)cc(C(N)=O)n3)c2)C1=O] | 6-[3-[2-[(3R)-3-hydroxy-1- methyl-2-oxo-pyrrolidin-3- yl]ethynyl]phenyl]-4-(2,2,2- trifluoroethoxy) pyridine-2-carboxamide |
| 190 | 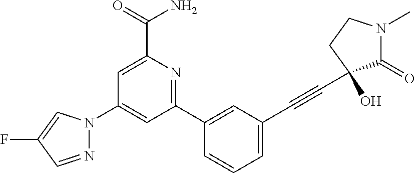 [SMILES: CN1CC[C@@](O)(C#Cc2cccc(-c3cc(-n4cc(F)cn4)cc(C(N)=O)n3)c2)C1=O] | 4-(4-fluoropyrazol-1-yl)-6-[3-[2- [(3R)-3-hydroxy-1-methyl-2- oxo-pyrrolidin-3-yl]ethynyl] phenyl]pyridine-2-carboxamide |
| 191 | 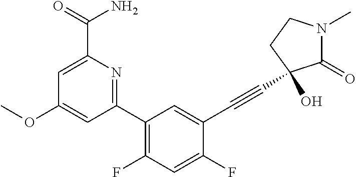 | 6-[2,4-difluoro-5-[2-[(3R)-3- hydroxy-1-methyl-2-oxo- pyrrolidin-3-yl]ethynyl] phenyl]-4-methoxy- pyridine-2-carboxamide |
| 192 | 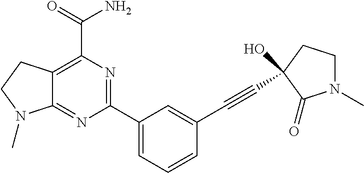 [SMILES: CN1CC[C@@](O)(C#Cc2cccc(-c3nc(C(N)=O)c4c(n3)N(C)CC4)c2)C1=O] | 2-[3-[2-[(3R)-3-hydroxy-1- methyl-2-oxo-pyrrolidin-3- yl]ethynyl]phenyl]-7-methyl- 5,6-dihydropyrrolo[2,3-d] pyrimidine-4-carboxamide |
| 193 | 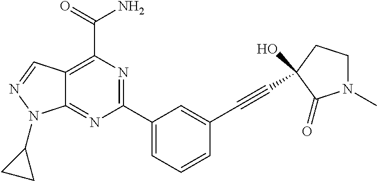 [SMILES: CN1CC[C@@](O)(C#Cc2cccc(-c3nc(C(N)=O)c4cnn(C5CC5)c4n3)c2)C1=O] | 1-cyclopropyl-6-[3-[2-[(3R)-3- hydroxy-1-methyl-2-oxo- pyrrolidin-3-yl]ethynyl]phenyl] pyrazolo[3,4-d]pyrimidine-4- carboxamide |
| 194 | 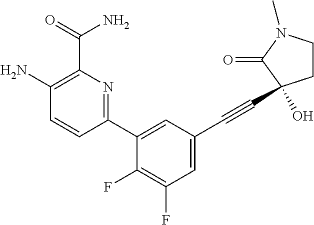 [SMILES: CN1CC[C@@](O)(C#Cc2cc(F)c(F)c(-c3ccc(N)c(C(N)=O)n3)c2)C1=O] | 3-amino-6-[2,3-difluoro-5-[2- [(3R)-3-hydroxy-1-methyl-2- oxo-pyrrolidin-3-yl]ethynyl] phenyl]pyridine-2-carboxamide |
| 195 | 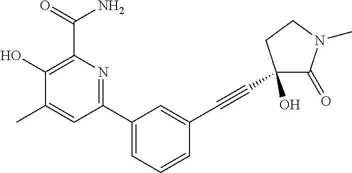 [SMILES: Cc1cc(-c2cccc(C#C[C@]3(O)CCN(C)C3=O)c2)nc(C(N)=O)c1O] | 3-hydroxy-6-[3-[2-[(3R)-3- hydroxy-1-methyl-2-oxo- pyrrolidin-3- yl]ethynyl]phenyl]-4- methyl-pyridine-2-carboxamide |
| 196 | 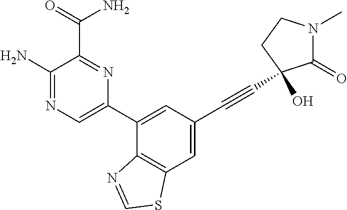 [SMILES: CN1CC[C@@](O)(C#Cc2cc(-c3cnc(N)c(C(N)=O)n3)c3ncsc3c2)C1=O] | 3-amino-6-[6-[2-[(3R)-3- hydroxy-1-methyl-2-oxo- pyrrolidin-3-yl]ethynyl]- 1,3-benzothiazol-4-yl] pyrazine-2-carboxamide |
| 197 | 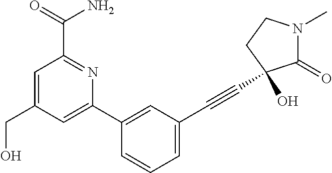 [SMILES: CN1CC[C@@](O)(C#Cc2cccc(-c3cc(CO)cc(C(N)=O)n3)c2)C1=O] | 4-(hydroxymethyl)-6-[3-[2- [(3R)-3-hydroxy-1-methyl- 2-oxo-pyrrolidin-3- yl]ethynyl]phenyl] pyridine-2-carboxamide |
| 198 | 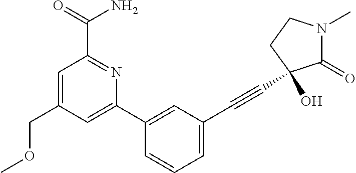 | 6-[3-[2-[(3R)-3-hydroxy-1- methyl-2-oxo-pyrrolidin-3- yl]ethynyl]phenyl]-4- (methoxymethyl) pyridine-2-carboxamide |
| 199 | 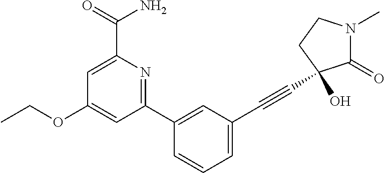 [SMILES: CCOc1cc(C(N)=O)nc(-c2cccc(C#C[C@]3(O)CCN(C)C3=O)c2)c1] | 4-ethoxy-6-[3-[2-[(3R)-3- hydroxy-1-methyl-2-oxo- pyrrolidin-3-yl]ethynyl] phenyl]pyridine-2- carboxamide |
| 200 | 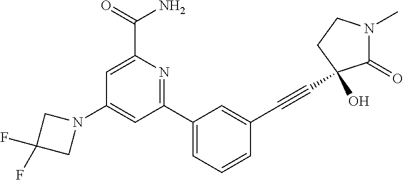 [SMILES: CN1CC[C@@](O)(C#Cc2cccc(-c3cc(N4CC(F)(F)C4)cc(C(N)=O)n3)c2)C1=O] | 4-(3,3-difluoroazetidin-1-yl)-6- [3-[2-[(3R)-3-hydroxy-1-methyl- 2-oxo-pyrrolidin-3-yl]ethynyl] phenyl]pyridine-2-carboxamide |

In some embodiments, the invention provides a compound of Examples A-Q6 below.

### Synthesis of NIK Inhibitors

Methods for preparing intermediates and compounds of the present invention are presented in the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives or reaction conditions. In addition, many of the compounds prepared by the methods described below can be further modified in light of this disclosure using conventional chemistry well known to those skilled in the art.

The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilstein's Handbuch der organishcen chemie, 4, Aufl. Ed. Springer-Verlag, Berlin including supplements also included via the Beilstein online database.

In preparing compounds of Formula (0), protection of remote functionality (e.g., primary or secondary amine) of intermediates may be necessary. The need for such protection will vary depending on the nature of the remote functionality and the conditions of the preparation methods. The need for such protection is readily determined by one skilled in the art. Exemplary protecting groups are provided herein. For a general description of protecting groups and their use, see T. W. Greene, Protective Groups in Organic Synthesis, John Wiley & Sons, New York, 1991.

Diastereomeric mixtures can be separated into their individual diastereoisomers on the basis of their physical chemical differences by methods well known to those skilled in the art, such as by chromatography or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column or supercritical fluid chromatography.

A single stereoisomer, e.g., an enantiomer, substantially free of its stereoisomer may be obtained by resolution of the racemic mixture using a method such as formation of diastereomers using optically active resolving agents (Eliel, E. and Wilen, S., Stereochemistry of Organic Compounds, John Wiley & Sons, Inc., New York, 1994; Lochmuller, C. H., J. Chromatogr., 113(3):283-302 (1975)). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Drug Stereochemistry, Analytical Methods and Pharmacology, Irving W. Wainer, Ed., Marcel Dekker, Inc., New York (1993).

Diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, the substrate to be resolved is reacted with one enantiomer of a chiral compound to form a diastereomeric pair (Eliel, E. and Wilen, S., Stereochemistry of Organic Compounds, John Wiley & Sons, Inc., New York, 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob, J. Org. Chem. 47:4165 (1982)), of the racemic mixture, and analyzing the NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (Chiral Liquid Chromatography W. J. Lough, Ed., Chapman and Hall, New York, (1989); Okamoto, J. of Chromatogr. 513:375-378 (1990)). Enriched or purified enantiomers can be distinguished by methods used to distinguish other chiral molecules with asymmetric carbon atoms, such as optical rotation and circular dichroism. The absolute stereochemistry of chiral centers and enatiomers can be determined by x-ray crystallography.

Positional isomers, for example E and Z forms, of compounds of Formula I and intermediates for their synthesis, may be observed by characterization methods such as NMR and analytical HPLC. For certain compounds where the energy barrier for interconversion is sufficiently high, the E and Z isomers may be separated, for example by preparatory HPLC.

Pharmaceutical Compositions and Administration

The compounds with which the invention is concerned are NIK kinase inhibitors, and are useful in the treatment of several diseases, for example, cancer or inflammatory conditions.

The invention also provides for compositions and medicaments comprising a compound of Formula (0) and at least one pharmaceutically acceptable carrier, diluent or excipient. The compositions of the invention can be used for inhibiting NF-kB signaling activity in mammals (e.g, human patients), by for example, inhibiting NIK activity.

By “pharmaceutically acceptable” it is meant the carrier, diluent or excipient must be compatible with the other ingredients of the formulation and not deleterious to the recipient thereof

In one embodiment, the invention provides for pharmaceutical compositions (or medicaments) comprising a compound of Formula (0) and a pharmaceutically acceptable carrier, diluent or excipient. In another embodiment, the invention provides for preparing compositions (or medicaments) comprising compounds of the invention. In another embodiment, the invention provides for administering compounds of Formula (0) and compositions comprising compounds of Formula (0) to a mammal (e.g., a human patient) in need thereof.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The effective amount of the compound to be administered will be governed by such considerations, and is the minimum amount necessary to inhibit NIK activity as required to prevent or treat the undesired disease or disorder, such as for example, neurodegeneration, amyloidosis, formation of neurofibrillary tangles, or undesired cell growth (e.g., cancer cell growth). For example, such amount may be below the amount that is toxic to normal cells, or the mammal as a whole.

In one example, the therapeutically effective amount of the compound of the invention administered parenterally per dose will be in the range of about 0.01-100 mg/kg, alternatively about e.g., 0.1 to 20 mg/kg of patient body weight per day, such as 0.3 to 15 mg/kg/day. The daily does is, in certain embodiments, given as a single daily dose or in divided doses two to six times a day, or in sustained release form. In the case of a 70 kg adult human, the total daily dose will generally be from about 7 mg to about 1,400 mg. This dosage regimen may be adjusted to provide the optimal therapeutic response. The compounds may be administered on a regimen of 1 to 4 times per day, preferably once or twice per day.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compositions comprising compounds of Formula (0) are normally formulated in accordance with standard pharmaceutical practice as a pharmaceutical composition. A typical formulation is prepared by mixing a compound of the present invention and a diluent, carrier or excipient. Suitable diluents, carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

Suitable carriers, diluents and excipients are well known to those skilled in the art and include materials such as carbohydrates, waxes, water soluble or swellable polymers, hydrophilic or hydrophobic materials, gelatin, oils, solvents, water and the like. The particular carrier, diluent or excipient used will depend upon the means and purpose for which a compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations can also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); or non-ionic surfactants such as TWEEN™, PLURONICS™ or polyethylene glycol (PEG). A active pharmaceutical ingredient of the invention (e.g., compound of Formula (0)) can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy: Remington the Science and Practice of Pharmacy (2005) 21^{st} Edition, Lippincott Williams & Wilkins, Philidelphia, Pa.

Sustained-release preparations of a compound of the invention can be prepared. Suitable examples of sustained-release preparations include semipermeable matrices of solid hydrophobic polymers containing a compound of Formula (0), which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

The formulations include those suitable for the administration routes detailed herein. The formulations can conveniently be presented in unit dosage form and can be prepared by any of the methods well known in the art of pharmacy. Techniques and formulations generally are found in Remington: The Science and Practice of Pharmacy: Remington the Science and Practice of Pharmacy (2005) 21^{st} Edition, Lippincott Williams & Wilkins, Philidelphia, Pa. Such methods include the step of bringing into association the active ingredient with the carrier which constitutes one or more accessory ingredients.

In general the formulations are prepared by uniformly and intimately bringing into association the active ingredient with liquid carriers, diluents or excipients or finely divided solid carriers, diluents or excipients, or both, and then, if necessary, shaping the product. A typical formulation is prepared by mixing a compound of the present invention and a carrier, diluent or excipient. The formulations can be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. A compound of the present invention is typically formulated into pharmaceutical dosage forms to provide an easily controllable dosage of the drug and to enable patient compliance with the prescribed regimen.

In one example, compounds of Formula (0) may be formulated by mixing at ambient temperature at the appropriate pH, and at the desired degree of purity, with physiologically acceptable carriers. The pH of the formulation depends mainly on the particular use and the concentration of compound, but typically ranges anywhere from about 3 to about 8. In one example, a compound of Formula (0) is formulated in an acetate buffer, at pH 5. In another embodiment, the compounds of Formula (0) are sterile. The compound may be stored, for example, as a solid or amorphous composition, as a lyophilized formulation or as an aqueous solution.

Formulations of a compound of the invention suitable for oral administration can be prepared as discrete units such as pills, capsules, cachets or tablets each containing a predetermined amount of a compound of the invention.

Compressed tablets can be prepared by compressing in a suitable machine the active ingredient in a free-flowing form such as a powder or granules, optionally mixed with a binder, lubricant, inert diluent, preservative, surface active or dispersing agent. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs can be prepared for oral use. Formulations of a compound of the invention intended for oral use can be prepared according to any method known to the art for the manufacture of pharmaceutical compositions and such compositions can contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

An example of a suitable oral administration form is a tablet containing about 1 mg, 5 mg, 10 mg, 25 mg, 30 mg, 50 mg, 80 mg, 100 mg, 150 mg, 250 mg, 300 mg and 500 mg of the compound of the invention, or any range derivable therein, compounded with about 5-30 mg anhydrous lactose, about 5-40 mg sodium croscarmellose, about 5-30 mg polyvinylpyrrolidone (PVP) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5-400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredient can be employed with either a paraffinic or a water-miscible ointment base. Alternatively, the active ingredients can be formulated in a cream with an oil-in-water cream base.

If desired, the aqueous phase of the cream base can include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations can desirably include a compound which enhances absorption or penetration of the active ingredient through the skin or other affected areas. Examples of such dermal penetration enhancers include dimethyl sulfoxide and related analogs.

The oily phase of the emulsions of this invention can be constituted from known ingredients in a known manner. While the phase can comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions of a compound of the invention contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose or saccharin.

Formulations of a compound of the invention can be in the form of a sterile injectable preparation, such as a sterile injectable aqueous or oleaginous suspension. This suspension can be formulated according to the known art using those suitable dispersing or wetting agents and suspending agents which have been mentioned above. The sterile injectable preparation can also be a sterile injectable solution or suspension in a non-toxic parenterally acceptable diluent or solvent, such as a solution in 1,3-butanediol or prepared as a lyophilized powder. Among the acceptable vehicles and solvents that can be employed are water, Ringer's solution and isotonic sodium chloride solution. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables.

The amount of active ingredient that can be combined with the carrier material to produce a single dosage form will vary depending upon the host treated and the particular mode of administration. For example, a time-release formulation intended for oral administration to humans can contain approximately 1 to 1000 mg of active material compounded with an appropriate and convenient amount of carrier material which can vary from about 5 to about 95% of the total compositions (weight:weight). The pharmaceutical composition can be prepared to provide easily measurable amounts for administration. For example, an aqueous solution intended for intravenous infusion can contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration can be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration can be prepared according to conventional methods and can be delivered with other therapeutic agents such as compounds heretofore used in the treatment of disorders as described below.

Formulations suitable for vaginal administration can be presented as pessaries, tampons, creams, gels, pastes, foams or spray formulations containing in addition to the active ingredient such carriers as are known in the art to be appropriate.

The formulations can be packaged in unit-dose or multi-dose containers, for example sealed ampoules and vials, and can be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

Indications and Methods of Treatment

The compounds of Formula (0) inhibit the activity of NIK. Accordingly, in another aspect of the invention the compounds of the invention can be used for the treatment of diseases and disorders in a mammal, for example a human patient, in which the inhibition of NIK in the patient would be therapeutically effective. For example, the compounds of the invention are useful for the treatment of diseases or disorders in a mammal (e.g., a human patient) associated with overactive or undesired NF-kB signaling through, for example, the overactivation of NIK. In one embodiment, the compounds of the invention are used to inhibit the activity of NIK, for example in an in vitro assay setting, by contacting said compound of Formula (0) with NIK. For example, compounds of Formula (0) can be used as a control compound in an in vitro assay setting.

In another embodiment, the compounds of the invention are used to inhibit the undesired signaling of NF-kB, e.g. in an cell proliferation assay, by introducing into a cell a compound of Formula (0). In another embodiment, the present invention provides the treatment of diseases or disorders in a mammal (e.g., human patient) associated with overactive or undesired NF-kB signaling (e.g., cancer, inflammatory diseases, among others) said method comprising administering to a mammal (e.g., a human patient) in need thereof a therapeutically effective amount of a compound of the invention.

Diseases and disorders treatable according to the methods of this invention include, cancer, inflammatory conditions, autoimmune disease and proliferation induced after medical procedures (e.g., arthritis, graft rejection, inflammatory bowel disease, cell proliferation induced after surgery angioplasty, among others). In one embodiment, a mammal (e.g., a human patient) is treated with a compound of the invention and a pharmaceutically acceptable carrier, adjuvant, or vehicle, wherein said compound of the invention is present in an amount to inhibit NF-kB signaling through, for example, but not limited to, inhibition of NIK.

In one embodiment, a compound of the invention can be used in the treatment of cell proliferative disorders.

In one embodiment of the invention, cancers that may be treated by the compounds of Formula (0) are selected from the group consisting of Lung (brochogenic carcinoma (non-small cell lung); Gatrointestinal—rectal, colorectal and colon; Genitourinary tract—kidney (papillary renal cell carcinoma); and skin—head and neck squamous cell carcinoma.

In one embodiment, compounds of Formula (0) can be use for the treatment of a cancer selected from the group consisting of head and neck squamous cell carcinomas, histiocytic lymphoma, lung adenocarcinoma, small cell lung cancer, non-small cell lung cancer, pancreatic cancer, papillary renal cell carcinoma, liver cancer, gastric cancers, colon cancer, leukemias, lymphomas, multiple myeloma, glioblastomas and breast carcinoma.

In one embodiment, compounds of Formula (0) can be used for the treatment of a cancer selected from the group consisting of histiocytic lymphoma, lung adenocarcinoma, small cell lung cancer, pancreatic cancer, liver cancer, gastric cancer, colon cancer, leukemias, lymphomas, multiple myeloma, glioblastomas and breast carcinoma.

In one embodiment, compound of Formula (0) can be used for the treatment of cancer selected from the group consisting of lymphomas, leukemias and multiple myeloma.

In one embodiment, the invention provides for the preparation of a medicament comprising a compound of Formula (0) for the treatment of lymphoma, leukemia or multiple myeloma.

In one embodiment, the invention provides for the treatment of lymphoma, leukemia or multiple myeloma, which method comprises administering an effective amount of a compound of Formula (0).

In one embodiment, compounds of the invention are useful for the treatment of inflammatory diseases and conditions including, but not limited to, lupus (including systemic lupus erythematosus, extra-renal lupus and lupus nephritis), asthma, COPD, rhinitis, multiple sclerosis, IBD, arthritis, gastritis, rheumatoid arthritis, dermatitis, endometriosis, transplant rejection, cardiac infarction, Alzheimer's diseases, diabetes Type II, inflammatory bowel disease, sepsis, and artherosclerosis.

In one embodiment, the invention provides for the use of a compound of Formula (0) for the treatment of an inflammatory condition.

In one embodiment, the invention provides for the use of a compound of Formula (0) for the preparation of a medicament for the treatment of an inflammatory condition.

In one embodiment, the invention provides for a compound of Formula (0) for the treatment of an inflammatory condition.

In one embodiment, the invention provides for a method for the treatment of an inflammatory condition, which method comprises administering an effective amount of a compound of Formula (0) to a patient in need thereof.

In one embodiment, the invention provides for the the treatment of an inflammatory condition selected from the group consisting of lupus (including systemic lupus erythematosus, extra-renal lupus and lupus nephritis), COPD, rhinitis, multiple sclerosis, IBD, arthritis, rheumatoid arthritis, dermatisis, endometriosis and transplant rejection, which method comprises administering an effective amount of a compound of Formula (0).

Combinations

The compounds of Formula I may be employed alone or in combination with other therapeutic agents for treatment. In one embodiment, compounds of this invention may be employed alone or in combination with chemotherapeutic agents. In one embodiment, compounds of this invention may be employed alone or in combination with anti-inflammatory agents. The compounds of the present invention can be used in combination with one or more additional drugs, for example an anti-inflammatory compound or anti-cancer compounds, that work by a different mechanism of action. The second compound of the pharmaceutical combination formulation or dosing regimen preferably has complementary activities to the compound of this invention such that they do not adversely affect each other. Such molecules are suitably present in combination in amounts that are effective for the purpose intended. The compounds may be administered together in a unitary pharmaceutical composition or separately and, when administered separately this may occur simultaneously or sequentially in any order. Such sequential administration may be close in time or remote in time.

In certain embodiments, a compound of Formula (0) is combined in a pharmaceutical combination formulation, or dosing regimen as combination therapy, with a second therapeutic compound that has anti-inflammatory or anti-cancer properties or that is useful for treating an inflammation, immune-response disorder, or hyperproliferative disorder (e.g., cancer). The second therapeutic agent may be a NSAID (Non-Steroidal Anti-Inflammatory Drug) or other anti-inflammatory agent. The second therapeutic agent may be a chemotherapeutic agent. In one embodiment, a pharmaceutical composition of this invention comprises a compound of Formula (0) in combination with a therapeutic agent such as an NSAID.

### EXAMPLES

The invention will be more fully understood by reference to the following examples. They should not, however, be construed as limiting the scope of the invention. These examples are not intended to limit the scope of the present invention, but rather to provide guidance to a skilled artisan to prepare and use the compounds, compositions, and methods of the present invention. While particular embodiments of the present invention are described, the skilled artisan will appreciate that various changes and modifications can be made without departing from the spirit and scope of the invention.

The chemical reactions in the Examples described can be readily adapted to prepare a number of other compounds of the invention, and alternative methods for preparing the compounds of this invention are deemed to be within the scope of this invention. For example, the synthesis of non-exemplified compounds according to the invention can be successfully performed by modifications apparent to those skilled in the art, e.g., by appropriately protecting interferring groups, by utilizing other suitable reagents known in the art other than those described, or by making routine modifications of reaction conditions. Alternatively, other reactions disclosed herein or known in the art will be recognized as having applicability for preparing other compounds of the invention.

Intermediates and compounds of the invention can be synthesized according to Schemes A-Y presented below, in which, R, R′, R″, R₁ and R₂ at each occurrence independently represents generally a non-interferring substituent (unless otherwise a specific substituent is specified in the description of the scheme); the symbol Ar at each occurrence represents independently an aromatic group; the symbol Het at each occurrence represents independently a heteroaryl group; and the symbol X at each occurrence represents independently any halogen (unless otherwise a specific halogen is specified in the description of the scheme).

General Procedure A:

SNAr

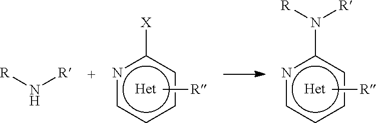

To a solution of nitrogen-containing nucleophile (1 eq.) and cesium carbonate (3.0 eq.) in N,N-dimethylformamide (2 mL/mmol) was added 2-haloheterocycle (1.1 eq.). The reaction was heated to 100° C. and stirred at this temperature for 2 hours. The reaction was then cooled to room temperature and acidified to pH=1 with 10% aqueous HCl solution if product contains a carboxylic acid, or diluted with water if neutral. The solution was extracted with twice with dichloromethane. The organic layers were combined, dried with sodium sulfate and concentrated under vacuum. The crude material was either used directly in subsequent reactions or purified by flash chromatography.

General Procedure B:

Amide Synthesis from Heterocyclic Carboxylic Acids

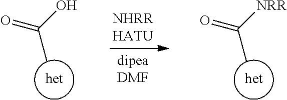

Aromatic or non-aromatic heterocyclic acid (1 eq) and HATU (1.2 eq) were weighed out and transferred to a vial to which DMF and DIPEA (3-5 eq) were subsequently added. The amine (HNRR) was added to the reaction mixture as a free base or HCl salt after a short period and the reaction was stirred at room temperature or at 50° C. for 2-18 hours. Reaction conversion was monitored by LCMS. Upon completion, the reaction was cooled and the crude product was triterated via addition of water and collected by filtration or extracted with sat ammonium chloride and DCM. Trituration or purification by chromatography gave the amide.

General Procedure C:

Chan-Lam Cross-Coupling

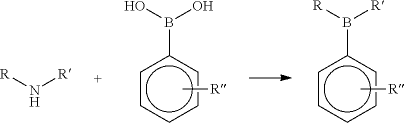

To a small vial was added the nitrogen-containing nucleophile (1 eq.), arylboronic acid (1.5 eq.), copper(II) acetate monohydrate (0.3 eq.) in N,N-dimethylformamide (2 mL/mmol) and pyridine (3.0 eq.). The reaction was stirred under an oxygen atmosphere at 90° C. for 6 hours. The reaction was then cooled to room temperature and diluted with a saturated aqueous sodium bicarbonate solution, and the aqueous phase was extracted with 3 times with dichloromethane. The organic phases were combined, washed with brine, dried with sodium sulfate and concentrated under vacuum. The crude material was purified by flash chromatography.

General Procedure D:

Hydrolysis of Nitrile to Primary Amide.

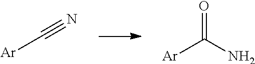

To a solution of an aryl nitrile (1 eq.) in ethanol (0.8 mL/mmol) and water (0.04 mL/mmol) was added hydrido(dimethylphosphinous acid-kp)[hydrogen bis(dimethylphosphinito-kp)]platinum(II) (0.05 eq.). The reaction was stirred at 90° C. for 2 h under air. The solution was then cooled to room temperature and extracted twice with ethyl acetate or dichloromethane. The organic layers were combined, dried with sodium sulfate and concentrated under vacuum. The crude material was either used directly in subsequent reactions or purified by flash chromatography.

General Procedures for aryl-halide (ArX) to terminal alkyne cross-coupling:

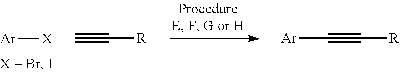

General Procedure E:

Aryl Halide was Weighed Out, Transferred to a Sealed Tube and brought up in Acetontrile (3 mL/mmol) and Triethylamine (3 mL/mmol). The solution was degassed with nitrogen and Copper(I) Iodide (0.05 eq) and Bis(triphenylphosphine)palladium(II)chloride (0.1 eq) were added. DMF (3 mL/mmol) was then added followed by dropwise addition of alkyne (2-3 eq). The reaction mixture heated for 3-18 h at 80° C. and monitored by LCMS for consumption of starting material. Upon completion, the reaction was cooled and the crude product was either triterated via addition of water and collected by filtration or extracted with saturated ammonium chloride and DCM whereupon the organic layer was dried, filtered and concentrated to dryness. Crude products were submitted for reverse phase HPLC purification.

General Procedure F:

Aryl halide (where X=bromide) (1 eq), Copper (I) Iodide (0.06 eq), tri-t-butylphosphonium tetrafluoroborate (0.2 eq) and dichlorobis(phenyl cyanide)palladium (0.1 eq) were weighed out and transferred to a microwave vessel. Upon addition of DMSO (3 mL/mmol), the reaction mixture was subsequently degassed whereupon a solution of alkyne (3 eq) in Diisopropylamine (3 eq) was added dropwise. The reaction mixture was capped and heated thermally at 80° C. and monitored by LCMS for consumption of starting material. Workup is the same for as in procedure E.

General Procedure G:

Aryl halide (wherein X=bromide) was weighed out, transferred to a sealed tube and brought up in DMSO or DMF (3 mL/mmol) and Triethylamine (3 mL/mmol). The solution was degassed with nitrogen and Bis(triphenylphosphine)palladium(II)chloride (0.2 eq) and alkyne (2-3 eq) were added (“copper-free” conditions). The reaction mixture heated for 2-18 hrs at 80° C. and monitored by LCMS for consumption of starting material. Workup is the same for as in above procedure E.

General Procedure H:

Ester to Amide Conversion with Sodium Methoxide/Formamide:

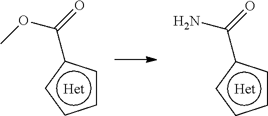

To a solution of Heterocyclic ester in N,N-Dimethylformamide was added formamide (10 eq) followed by dropwise addition of sodium methoxide (3 eq). The mixture was either stirred at room temperature or heated to 40° C. and monitored by LC-MS for completion. The crude reaction mixture was triturated via addition of saturated ammonium chloride or extracted with Dichloromethane in cases where the product did not crash out. In situations where this was an intermediate, the crude material was used directly in subsequent reactions.

General Procedure I:

Ester to Amide Conversion with Ammonium Hydroxide in Dioxane:

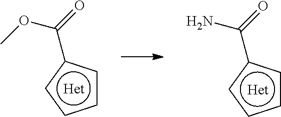

To a solution of Heterocyclic ester in dioxane (l0 mL/mmol) was added ammonium hydroxide (25% mass) in water (50 eq., 14 mmol). The reaction mixture was stirred at 40° C. and monitored by LC-MS for completion. The crude reaction mixture was concentrated to dryness and purified by reverse phase HPLC to afford product.

General Procedure J:

Ester Saponification:

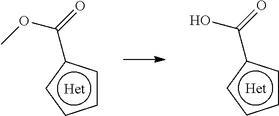

To a solution of heterocylic ester in 1:1 Tetrahydrofuran/Water was added lithium hydroxide monohydrate (3-10 eq). The reaction was either stirred at room temperature or heated to 50° C. and monitored by LC-MS for completion. The tetrahydrofuran was then evaporated and the pH of the aqueous crude reaction mixture was adjusted to 3 whereupon the product either crashed out and was isolated, or the aqueous layer was extracted with Dichloromethane or ethyl acetate in cases where the product did not crash out. In situations where this was an intermediate, the crude material was used directly in subsequent reactions.

General Procedure K:

Ketone/Aldehyde Reduction:

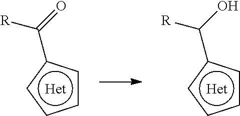

To a solution of heterocylic ketone/aldehyde in Methanol was added sodium borohydride (1-3 eq). The reaction was stirred at 0° C. or room temperature until bubbling subsided and monitored by LC-MS for completion. The reaction mixture was extracted with dichloromethane and saturated ammonium chloride whereupon the organic layer was dried, filtered and concentrated to afford crude heterocylic alcohol intermediate and was used directly in subsequent reactions.

General Procedure L:

Fluorination.

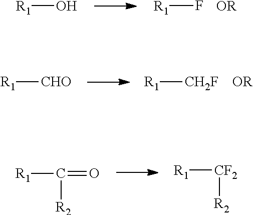

To a solution of alcohol, aldehyde or ketone in Dichloromethane or Dichloroethane was added 4 equivalents of Diethylaminosulfur trifluoride (DAST) or Bis(2-methoxyethyl)aminosulfur trifluoride (Deoxo-Fluor). The reaction was either stirred at room temperature or heated to 45° C. and monitored by LC-MS for completion. The reaction mixture was concentrated to dryness and the crude intermediate was triturated via addition of water which was used in subsequent reactions without further purification.

General Procedure M:

Suzuki Coupling of Boronic Acids or Boronic Esters with Aryl Halides.

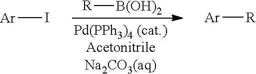

Aryl Halide, tetrakis (triphenylphosphine)palladium or Palladium (II) bis(triphenylphosphine) dichloride (0.05 eq) and boronic acid or pinnacol ester (1.2 eq) were weighed out into a microwave vessel or sealed tube. Acetonitrile (3 mL/mmol) and a 1M aqueous solution of Sodium Carbonate (3 eq) were added. The vessel was capped and heated thermally 3-18 hrs at 100° C. Upon completion, the reaction was cooled and crude product was either triterated via addition of water and collection by filtration or extracted with sat ammonium chloride and DCM. If the crude product was an intermediate, it was taken into the next step in most cases w/o further purification or alternatively submitted for reverse phase HPLC purification when it was a final product.

General Procedure N:

Reductive Amination of Arylaldehydes.

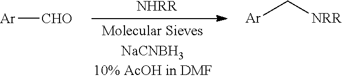

To a vial containing aryl aldehyde (1 eq) in 10% Acetic Acid in DMF (6 mL/mmol) was added molecular sieves (1 eq by wt), amine (HNR¹R², 4 eq) then sodium cyanoborohydride (1.2 eq). The reaction was either heated at 45° C. or stirred at room temperature. Upon completion, the reaction was extracted with DCM and saturated ammonium chloride. The organic layer was dried with magnesium sulfate, filtered and concentrated to give crude product which was taken into the next step without purification.

General Procedure O:

Carbonylative Methanolysis of Aryl Iodides.

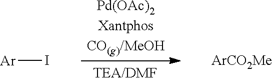

To a nitrogen-purged solution of aryl iodide in TEA (3 mL/mmol), DMF (3 mL/mmol) and MeOH (3 mL/mmol) was added Palladium (III)Acetate (0.03 eq) and Xantphos (0.06 eq). The reaction mixture was flushed with Carbon Monoxide gas for several minutes and then sealed with CO balloon attached and heated to 60° C. for 3 hours. Upon completion, the reaction was cooled to room temperature and the crude product was triterated via addition of water and collected by filtration. The crude interemediate was taken into the next step w/o further purification.

General Procedure P:

Carbonylative Amidation with HMDS.

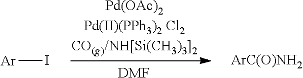

To a nitrogen-degassed solution of generic aryl iodide (Ar—I) in DMF (170 eq) was added Palladium(II)bis(triphenylphosphine) dichloride (0.05 eq) and hexamethyldisilazane (6 eq). The reaction mixture was flushed with Carbon Monoxide gas for several minutes and then sealed with CO balloon attached and heated to 70° C. for 18 hrs. Upon completion, the reaction was cooled to room temperature and the crude was triterated via addition of water and collected by filtration. The crude interemediate was taken into the next step w/o further purification.

General Procedure Q:

Stille Reaction, Aryl Halo to Aryl Vinylether Conversion

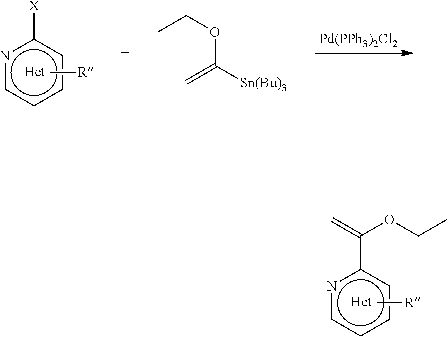

Under nitrogen, a suspension of aryl halo (1 eq.), tributyl(1-ethoxyethenyl)stannane (1 eq.), Pd(PPh₃)₂Cl₂ (0.1 eq.) in N, N-dimethylformamide was stirred at about 60° C. for 1˜10 h. After cooling, the reaction was quenched by saturated potassium fluoride aqueous solution and diluted with ethyl acetate. The precipitate was filtered off and the filtrate was collected and washed with water and brine. The organic layer was dried over anhydrous sodium sulfate and concentrated under vacuum. The residue was purified by silica gel column chromatography.

General Procedure R:

Oxidation, Aryl Vinyl Ether to Ester Conversion

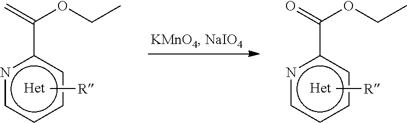

A solution of sodium periodate (2 eq.) in water was added to a solution of aryl vinyl ether (1 eq.) in 1,4-dioxane. Then potassium permanganate (0.5 eq.) was added and the mixture was stirred for 1-10 h at room temperature. After completion, the mixture was adjusted to pH 7-8 with saturated potassium carbonate solution. The precipitate was filtered off and the residue was rinsed thoroughly with dichloromethane. The combined filtrates were washed with water and the organic layer was dried over anhydrous sodium sulfate and concentrated under vacuum. The residue was purified by silica gel column chromatography.

General Procedure S:

Ester to Amide Conversion Using Ammonia in Methanol

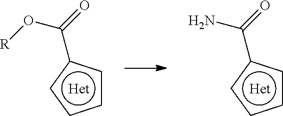

To a stirred solution of ester (1 equiv) in methanol was treated with saturated ammonia (>20 eq.) in methanol. The mixture was either stirred at room temperature or heated to 40° C. and the reaction was monitored by LC-MS. The crude reaction mixture was concentrated and purified by reverse phase HPLC.

General Procedure T:

SEM Deprotection with HCl

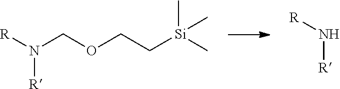

The SEM-protected amine or alcohol and 4.0 M hydrochloric acid in dioxane (17.0 eq.) were combined in ethanol (4.0 mL/mmol) and stirred at 50° C. for 2 h. The sample was then concentrated under vacuum and used directly in subsequent reactions or purified by flash chromatography.

General Procedures for Suzuki Couplings with Aryltrifluoroborates:

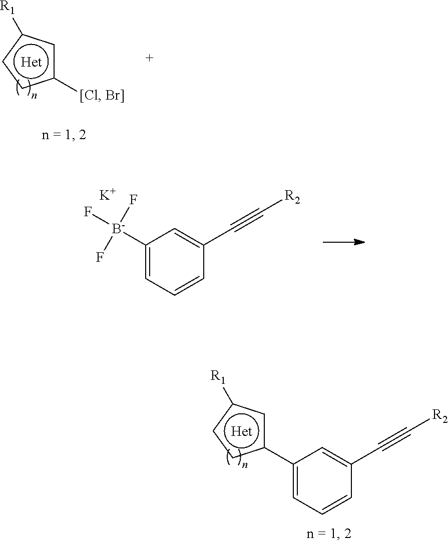

General Procedure U:

A tube containing a solution of arylchloride/bromide (1 eq) and aryltrifluoroborate (1 eq) in Ethanol was purged with nitrogen before addition of Pd(OAc)2 (0.06 eq), RuPhos (0.12 eq), and Sodium Carbonate (2 eq). The tube was sealed with a cap lined with a disposable Teflon septum was heated at 85° C. for 12-20 hours. The reaction mixture was allowed to cool to room temperature and was either filtered through Celite® and submitted directly to reverse phase HPLC purification or extracted with dichloromethane and a solution of saturated ammonium chloride before drying, evaporating and submitting to reverse phase purification or using in the subsequent step without purification.

General Procedure V: A solution of arylchloride/bromide (1 eq) and aryltrifluoroborate (1 eq) in 20% aq dioxane (0.28 M) was degassed before addition of cesium carbonate (3 eq) and tetrakis(triphenylphosphine)palladium(0) (0.05 eq). The reaction mixture was heated at 100° C. for 1 hr then cooled to room temperature. Workup same as General Procedure U.

General Procedure W:

A solution of arylchloride/bromide (1 eq) and aryltrifluoroborate (1 eq) in Acetonitrile (0.25M) was degassed before addition of tetrakis(triphenylphosphine)palladium(0) (0.05 eq) and a 1:1 mixture of 1M Sodium Carbonate (2 eq) and 1M Potassium Acetate (2 eq). The reaction was performed in a 5 mL Biotage® microwave tube and heated to 140° C. for 20-40 minutes then cooled to room temperature. Workup same as General Procedure U.

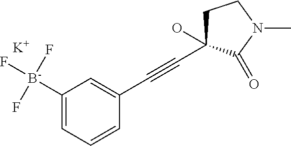 [SMILES: CN1CC[C@](O)(C#Cc2cccc([B-](F)(F)F)c2)C1=O ; [K+]]

Potassium trifluoro-(3-iodophenyl)boranuide (1 eq) is brought up in a solution of 1:1 Triethylamine (14 eq) and N,N-dimethylformamide (26 eq). The solution was purged with nitrogen before addition of cuprous iodide (0.05 eq), bis(triphenylphosphine)palladium(II) dichloride (0.05 eq) and (3R)-3-ethynyl-3-hydroxy-1-methyl-pyrrolidin-2-one (1.05 eq) at once. The reaction mixture was stirred at 40° C. overnight (18 hrs) whereupon the reaction mixture was concentrated under vacuum to yield a dark brown oil. Water was added and the solution was sonicated until an orange-brown solid crashed out of solution. The solid was filtered off and the aqueous layer was concentrated under high vacuum to afford a dark red sludge. The sludge was azeotroped 3 times with Hexanes, brought up in Methanol, sonicated and the subsequent light brown solid was then filtered and collected to afford potassium (S)-trifluoro(3-((3-hydroxy-1-methyl-2-oxopyrrolidin-3yl)ethynyl)phenyl)borate in a 80% yield.

### Example A

### Synthesis of 2-[3-[2-[(3R)-3-hydroxy-1-methyl-2-oxo-pyrrolidin-3-yl]ethynyl]phenyl]pyrimidine-4-carboxamide

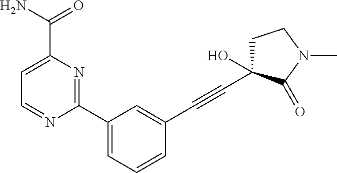 [SMILES: CN1CC[C@@](O)(C#Cc2cccc(-c3nccc(C(N)=O)n3)c2)C1=O]

A tube containing a solution of 2-chloropyrimidine-4-carboxamide (0.24 g, 1 eq) and potassium (S)-trifluoro(3-((3-hydroxy-1-methyl-2-oxopyrrolidin-3yl)ethynyl)phenyl)borate (1 eq) in Ethanol (0.25 M) was purged with nitrogen before addition of Pd(OAc)₂ (0.06 eq), RuPhos (0.12 eq), and Sodium Carbonate (2 eq). The tube was sealed and stirred at 85° C. for 18 hours. The reaction mixture was cooled to room temperature and extracted with dichloromethane and saturated ammonium chloride then dried with Magnesium sulfate, filtered and concentrated to dryness. The crude material subjected to reverse phase purification to afford 53 mg of the title compound (10%). M+H=337.0; 1H NMR (400 MHz, DMSO-d6) δ 9.16-9.11 (m, 1H), 8.70-8.60 (m, 3H), 7.98 (s, 1H), 7.94 (d, J=5.0 Hz, 1H), 7.64-7.54 (m, 2H), 6.47 (s, 1H), 3.40-3.35 (m, 2H), 2.81 (s, 3H), 2.48-2.43 (m, 1H), 2.25-2.17 (m, 1H).

### Example B

### Synthesis of 6-[3-[2-[(3R)-3-hydroxy-1-methyl-2-oxo-pyrrolidin-3-yl]ethynyl]phenyl]pyridine-2-carboxamide

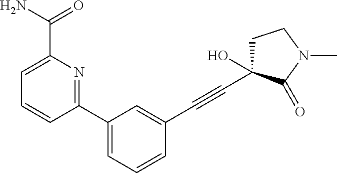 [SMILES: CN1CC[C@@](O)(C#Cc2cccc(-c3cccc(C(N)=O)n3)c2)C1=O]

Similar to as described in General Procedure U, 6-bromopyridine-2-carboxamide was reacted with (S)-trifluoro(3-((3-hydroxy-1-methyl-2-oxopyrrolidin-3yl)ethynyl)phenyl)borate to give 30 mg of the title compound (24%). M+H=356.0; 1H NMR (400 MHz, DMSO-d6) δ 8.37 (s, 1H), 8.36-8.33 (m, 1H), 8.31-8.29 (m, 1H), 8.22 (dd, J=7.9, 1.1 Hz, 1H), 8.09-8.04 (m, 1H), 8.00 (dd, J=7.7, 1.1 Hz, 1H), 6.49 (s, 1H), 3.40-3.35 (m, 2H), 2.81 (s, 3H), 2.49-2.43 (m, 1H), 2.24-2.16 (m, 1H).

### Example C

### Synthesis of 4-[3-[2-[(3R)-3-hydroxy-1-methyl-2-oxo-pyrrolidin-3-yl]ethynyl]phenyl]pyrimidine-2-carboxamide

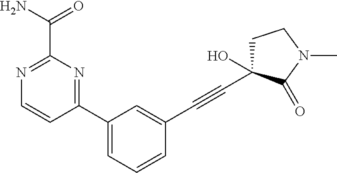 [SMILES: CN1CC[C@@](O)(C#Cc2cccc(-c3ccnc(C(N)=O)n3)c2)C1=O]

Similar to as described in General Procedure V, 4-bromopyrimidine-2-carbonitrile (100 mg) was reacted with (S)-trifluoro(3-((3-hydroxy-1-methyl-2-oxopyrrolidin-3yl)ethynyl)phenyl)borate (1.5 eq), cesium carbonate (3 eq) and tetrakis(triphenylphosphine)palladium(0) (0.05 eq) for 1 hour at 100° C. to afford 4-[3-[2-[(3R)-3-hydroxy-1-methyl-2-oxo-pyrrolidin-3-yl]ethynyl]phenyl]pyrimidine-2-carbonitrile following extraction. This intermediate was taken onto the next step without purification. Similar to as described in General Procedure D, 4-[3-[2-[(3R)-3-hydroxy-1-methyl-2-oxo-pyrrolidin-3-yl]ethynyl]phenyl]pyrimidine-2-carbonitrile was reacted to afford 23 mg of the title compound (45%). M+H=337.0; 1H NMR (400 MHz, DMSO-d6) δ 9.00 (d, J=5.3 Hz, 1H), 8.42-8.33 (m, 3H), 8.27 (d, J=5.4 Hz, 1H), 7.83 (s, 1H), 7.66-7.57 (m, 2H), 6.49 (s, 1H), 3.41-3.34 (m, 2H), 2.81 (s, 3H), 2.48-2.43 (m, 1H), 2.25-2.16 (m, 1H).

### Example D

### Synthesis of 2-[3-[2-[(3R)-3-hydroxy-1-methyl-2-oxo-pyrrolidin-3-yl]ethynyl]phenyl]quinazoline-4-carboxamide

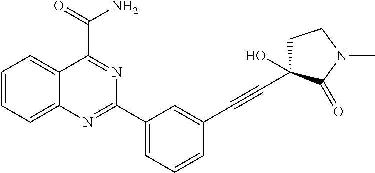 [SMILES: CN1CC[C@@](O)(C#Cc2cccc(-c3nc(C(N)=O)c4ccccc4n3)c2)C1=O]

Similar to as described in General Procedure U, ethyl 2-chloroquinazoline-4-carboxylate (0.1 g) was reacted with (S)-trifluoro(3-((3-hydroxy-1-methyl-2-oxopyrrolidin-3yl)ethynyl)phenyl)borate to give ethyl 2-[3-[2-[(3R)-3-hydroxy-1-methyl-2-oxo-pyrrolidin-3-yl]ethynyl]phenyl]quinazoline-4-carboxylate. This crude material was subsequently suspended in 1,4 dioxane (75 eq) whereupon Ammonium Hydroxide (50 eq) was added and the solution was heated at 50° C. until the reaction was complete. The reaction was concentrated to dryness and subjected to reverse phase purification to give 36 mg of the title compound (22%). M+H=387.0; 1H NMR (400 MHz, DMSO-d6) δ 8.79-8.75 (m, 1H), 8.71-8.64 (m, 2H), 8.63 (s, 1H), 8.19-8.14 (m, 1H), 8.11 (s, 1H), 8.11-8.06 (m, 1H), 7.83-7.76 (m, 1H), 7.65-7.60 (m, 2H), 6.49 (s, 1H), 3.38 (dd, J=7.1, 5.8 Hz, 2H), 2.82 (s, 3H), 2.48-2.45 (m, 1H), 2.26-2.18 (m, 1H).

### Example E

### Synthesis of 2-[3-[2-[(3R)-3-hydroxy-1-methyl-2-oxo-pyrrolidin-3-yl]ethynyl]phenyl]-6-morpholino-pyrimidine-4-carboxamide

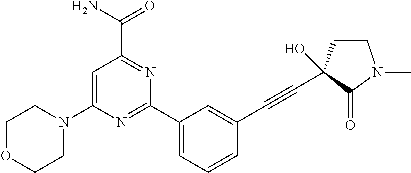 [SMILES: CN1CC[C@@](O)(C#Cc2cccc(-c3nc(C(N)=O)cc(N4CCOCC4)n3)c2)C1=O]

Similar to as described in General Procedure A, methyl 2,6-dichloropyrimidine-4-carboxylate (0.5 g) was reacted with morpholine to give methyl 2-chloro-6-morpholino-pyrimidine-4-carboxylate (0.28 g) following trituration. Similar to as described in General Procedure U, methyl 2-chloro-6-morpholinopyrimidine-4-carboxylate (75 mg) was reacted with (S)-trifluoro(3-((3-hydroxy-1-methyl-2-oxopyrrolidin-3yl)ethynyl)phenyl)borate to give ethyl 2-[3-[2-[(3R)-3-hydroxy-1-methyl-2-oxo-pyrrolidin-3-yl]ethynyl]phenyl]-6-morpholinopyrimidine-4-carboxylate. This intermediate was taken onto the next step without purification. Similar to as described in General Procedure I, ethyl 2-[3-[2-[(3R)-3-hydroxy-1-methyl-2-oxo-pyrrolidin-3-yl]ethynyl]phenyl]-6-morpholino-pyrimidine-4-carboxylate was reacted to give 22.4 mg of the title compound (18%). M+H=422.0; 1H NMR (400 MHz, DMSO-d6) δ 8.58-8.54 (m, 1H), 8.50 (d, J=1.5 Hz, 1H), 8.38 (s, 1H), 7.77 (s, 1H), 7.57-7.48 (m, 2H), 7.27 (s, 1H), 6.46-6.42 (m, 1H), 3.81-3.68 (m, 8H), 3.39-3.34 (m, 2H), 2.81 (s, 3H), 2.48-2.43 (m, 1H), 2.25-2.14 (m, 1H).

### Example F

### Synthesis of 2-[3-[2-[(3R)-3-hydroxy-1-methyl-2-oxo-pyrrolidin-3-yl]ethynyl]phenyl]-6-pyrrolidin-1-yl-pyrimidine-4-carboxamide

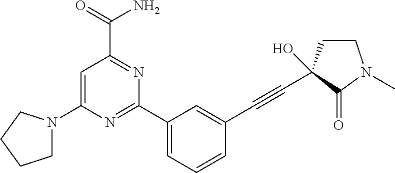 [SMILES: CN1CC[C@@](O)(C#Cc2cccc(-c3nc(C(N)=O)cc(N4CCCC4)n3)c2)C1=O]

Similar to as described in General Procedure A, 2,6-dichloropyrimidine-4-carboxamide (75 mg) was reacted with pyrrolidine to give 2-chloro-6-pyrrolidin-1-yl-pyrimidine-4-carboxamide. This intermediate was taken onto the next step without purification. Similar to as described in General Procedure W, 2-chloro-6-pyrrolidin-1-yl-pyrimidine-4-carboxamide was then reacted with (S)-trifluoro(3-((3-hydroxy-1-methyl-2-oxopyrrolidin-3yl)ethynyl)phenyl)borate to give 14 mg of the title compound (9%). M+H=406.0; 1H NMR (400 MHz, DMSO-d6) δ 8.58 (dt, J=7.4, 1.7 Hz, 1H), 8.51 (d, J=1.7 Hz, 1H), 8.34 (s, 1H), 7.75 (s, 1H), 7.58-7.46 (m, 2H), 6.96 (s, 1H), 6.44 (s, 1H), 3.77-3.63 (m, 2H), 3.55-3.42 (m, 2H), 3.39-3.33 (m, 2H), 2.81 (s, 3H), 2.48-2.41 (m, 1H), 2.25-2.15 (m, 1H), 2.07-1.93 (m, 4H).

### Example G

### Synthesis of 6-(dimethylamino)-2-[3-[2-[(3R)-3-hydroxy-1-methyl-2-oxo-pyrrolidin-3-yl]ethynyl]phenyl]pyrimidine-4-carboxamide

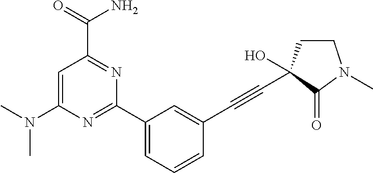

Similar to as described in General Procedure A, 2,6-dichloropyrimidine-4-carboxamide (75 mg) was reacted with dimethylamine Hydrochloride to give 2-chloro-6-(dimethylamino)pyrimidine-4-carboxamide. This intermediate was taken onto the next step without purification. Similar to as described in General Procedure W, 2-chloro-6-(dimethylamino)pyrimidine-4-carboxamide was then reacted with (S)-trifluoro(3-((3-hydroxy-1-methyl-2-oxopyrrolidin-3yl)ethynyl)phenyl)borate to give 19 mg of the title compound (13%). M+H=380.0; 1H NMR (400 MHz, DMSO-d6) δ 8.57 (dt, J=7.4, 1.7 Hz, 1H), 8.53-8.50 (m, 1H), 8.36 (s, 1H), 7.75 (s, 1H), 7.57-7.48 (m, 2H), 7.13 (s, 1H), 6.45 (s, 1H), 3.40-3.33 (m, 2H), 3.22 (s, 6H), 2.81 (s, 3H), 2.48-2.42 (m, 1H), 2.25-2.14 (m, 1H).

### Example H

### Synthesis of 6-(4-acetylpiperazin-1-yl)-2-[3-[2-[(3R)-3-hydroxy-1-methyl-2-oxo-pyrrolidin-3-yl]ethynyl]phenyl]pyrimidine-4-carboxamide

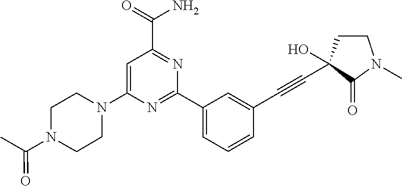 [SMILES: CC(=O)N1CCN(c2cc(C(N)=O)nc(-c3cccc(C#C[C@]4(O)CCN(C)C4=O)c3)n2)CC1]

Similar to as described in General Procedure A, 2,6-dichloropyrimidine-4-carboxamide (75 mg) was reacted with 1-acetylpiperazine to give 6-(4-acetylpiperazin-1-yl)-2-chloro-pyrimidine-4-carboxamide. This intermediate was taken onto the next step without purification. Similar to as described in General Procedure W, 6-(4-acetylpiperazin-1-yl)-2-chloro-pyrimidine-4-carboxamide was then reacted with (S)-trifluoro(3-((3-hydroxy-1-methyl-2-oxopyrrolidin-3yl)ethynyl)phenyl)borate to give 9 mg of the title compound (5%). M+H=463.0; 1H NMR (400 MHz, DMSO-d6) δ 8.57 (dt, J=7.5, 1.7 Hz, 1H), 8.52-8.49 (m, 1H), 8.39 (s, 1H), 7.78 (s, 1H), 7.58-7.48 (m, 2H), 7.29 (s, 1H), 6.45 (s, 1H), 3.91-3.74 (m, 4H), 3.64-3.57 (m, 4H), 3.41-3.34 (m, 2H), 2.81 (s, 3H), 2.49-2.43 (m, 1H), 2.25-2.16 (m, 1H), 2.07 (s, 3H).

### Example I

### Synthesis of 6-(3,3-difluoroazetidin-1-yl)-2-[3-[2-[(3R)-3-hydroxy-1-methyl-2-oxo-pyrrolidin-3-yl]ethynyl]phenyl]pyrimidine-4-carboxamide

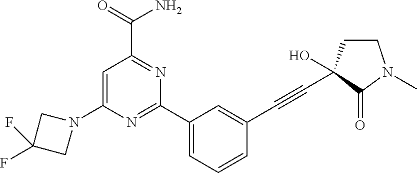 [SMILES: CN1CC[C@@](O)(C#Cc2cccc(-c3nc(C(N)=O)cc(N4CC(F)(F)C4)n3)c2)C1=O]

Similar to as described in General Procedure A, 2,6-dichloropyrimidine-4-carboxamide (75 mg) was reacted with 3,3-difluoroazetidine hydrochloride to give 2-chloro-6-(3,3-difluoroazetidin-1-yl)pyrimidine-4-carboxamide. This intermediate was taken onto the next step without purification. Similar to as described in General Procedure W, 2-chloro-6-(3,3-difluoroazetidin-1-yl)pyrimidine-4-carboxamide was then reacted with (S)-trifluoro(3-((3-hydroxy-1-methyl-2-oxopyrrolidin-3yl)ethynyl)phenyl)borate to give 12 mg of the title compound (7%). M+H=428.0; 1H NMR (400 MHz, DMSO-d6) δ 8.58 (dt, J=7.6, 1.6 Hz, 1H), 8.53-8.50 (m, 1H), 8.43 (s, 1H), 7.82 (s, 1H), 7.59-7.49 (m, 2H), 7.05 (s, 1H), 6.44 (s, 1H), 4.72-4.63 (m, 4H), 3.40-3.33 (m, 2H), 2.81 (s, 3H), 2.48-2.42 (m, 1H), 2.25-2.15 (m, 1H).

### Example J

### Synthesis of 6-(cyclopropylamino)-2-[3-[2-[(3R)-3-hydroxy-1-methyl-2-oxo-pyrrolidin-3-yl]ethynyl]phenyl]pyrimidine-4-carboxamide

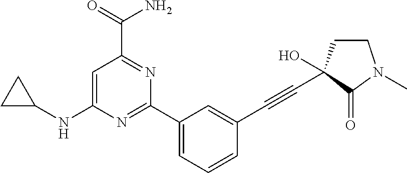 [SMILES: CN1CC[C@@](O)(C#Cc2cccc(-c3nc(NC4CC4)cc(C(N)=O)n3)c2)C1=O]

Similar to as described in General Procedure A, 2,6-dichloropyrimidine-4-carboxamide (75 mg) was reacted with cyclopropylamine to give 2-chloro-6-(cyclopropylamino)pyrimidine-4-carboxamide. This intermediate was taken onto the next step without purification. Similar to as described in General Procedure W, 2-chloro-6-(cyclopropylamino)pyrimidine-4-carboxamide was then reacted with (S)-trifluoro(3-((3-hydroxy-1-methyl-2-oxopyrrolidin-3yl)ethynyl)phenyl)borate to give 10 mg of the title compound (6%). M+H=392.0

### Example K

### Synthesis of 2-[3-[2-[(3R)-3-hydroxy-1-methyl-2-oxo-pyrrolidin-3-yl]ethynyl]phenyl]-6-[2-methoxyeth yl(methyl)amino]pyrimidine-4-carboxamide

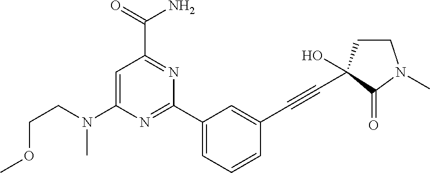

Similar to as described in General Procedure A, 2,6-dichloropyrimidine-4-carboxamide (75 mg) was reacted with N-(2-methoxyethyl)methylamine to give 2-chloro-6-(cyclopropylamino)pyrimidine-4-carboxamide. This intermediate was taken onto the next step without purification. Similar to as described in General Procedure W, 2-chloro-6-[2-methoxyethyl(methyl)amino]pyrimidine-4-carboxamide was then reacted with (S)-trifluoro(3-((3-hydroxy-1-methyl-2-oxopyrrolidin-3yl)ethynyl)phenyl)borate to give 39 mg of the title compound (23%). M+H=424.0; 1H NMR (400 MHz, DMSO-d6) δ 8.57-8.52 (m, 1H), 8.52-8.49 (m, 1H), 8.36 (s, 1H), 7.76 (s, 1H), 7.57-7.48 (m, 2H), 7.15 (s, 1H), 6.45 (s, 1H), 4.07-3.67 (m, 2H), 3.60 (t, J=5.5 Hz, 2H), 3.39-3.33 (m, 2H), 3.28 (s, 3H), 3.20 (s, 3H), 2.81 (s, 3H), 2.48-2.42 (m, 1H), 2.25-2.16 (m, 1H).

### Example L

### Synthesis of 2-[3-[2-[(3R)-3-hydroxy-1-methyl-2-oxo-pyrrolidin-3-yl]ethynyl]phenyl]-6-(6-oxa-2-azaspiro[3.3]heptan-2-yl)pyrimidine-4-carboxamide

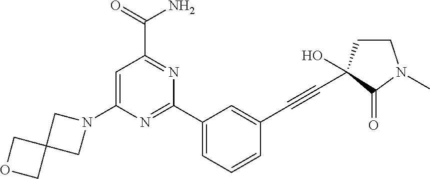 [SMILES: CN1CC[C@@](O)(C#Cc2cccc(-c3nc(C(N)=O)cc(N4CC5(COC5)C4)n3)c2)C1=O]

Similar to as described in General Procedure A, 2,6-dichloropyrimidine-4-carboxamide (75 mg) was reacted with 2-oxa-6-azaspiro[3.3]heptane to give 2-chloro-6-(6-oxa-2-azaspiro[3.3]heptan-2-yl)pyrimidine-4-carboxamide. This intermediate was taken onto the next step without purification. Similar to as described in General Procedure W, 2-chloro-6-(6-oxa-2-azaspiro[3.3]heptan-2-yl)pyrimidine-4-carboxamide was then reacted with (S)-trifluoro(3-((3-hydroxy-1-methyl-2-oxopyrrolidin-3yl)ethynyl)phenyl)borate to give 49 mg of the title compound (29%). M+H=434.0; 1H NMR (400 MHz, DMSO-d6) δ 8.55 (dt, J=7.5, 1.7 Hz, 1H), 8.50-8.47 (m, 1H), 8.35 (s, 1H), 7.76 (s, 1H), 7.57-7.48 (m, 2H), 6.84 (s, 1H), 6.45 (s, 1H), 4.80-4.71 (m, 4H), 4.41-4.31 (m, 4H), 3.40-3.33 (m, 2H), 2.81 (s, 3H), 2.47-2.41 (m, 1H), 2.24-2.16 (m, 1H).

### Example M

### Synthesis of 2-[3-[2-[(3R)-3-hydroxy-1-methyl-2-oxo-pyrrolidin-3-yl]ethynyl]phenyl]-6-(methylamino)pyrimidine-4-carboxamide

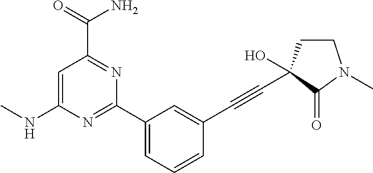

Similar to as described in General Procedure A, 2,6-dichloropyrimidine-4-carboxamide (75 mg) was reacted with methylamine hydrochloride to give 2-chloro-6-(methylamino)pyrimidine-4-carboxamide. This intermediate was taken onto the next step without purification. Similar to as described in General Procedure W, 2-chloro-6-(methylamino)pyrimidine-4-carboxamide was then reacted with (S)-trifluoro(3-((3-hydroxy-1-methyl-2-oxopyrrolidin-3yl)ethynyl)phenyl)borate to give 4 mg of the title compound (3%). M+H=366.0

### Example N

### Synthesis of 2-[3-[2-[(3R)-3-hydroxy-1-methyl-2-oxo-pyrrolidin-3-yl]ethynyl]phenyl]-6-methyl-pyrimidine-4-carboxamide

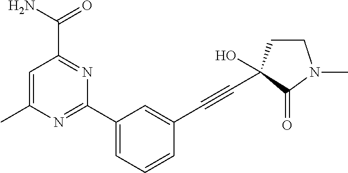

Similar to as described in General Procedure U, ethyl 2-chloro-6-methyl-pyrimidine-4-carboxylate (0.08 g) was reacted with (S)-trifluoro(3-((3-hydroxy-1-methyl-2-oxopyrrolidin-3yl)ethynyl)phenyl)borate to give ethyl 2-[3-[2-[(3R)-3-hydroxy-1-methyl-2-oxo-pyrrolidin-3-yl]ethynyl]phenyl]-6-methyl-pyrimidine-4-carboxylate. This crude material was taken on without further purification. Similar to as described in General Procedure I, ethyl 2-[3-[2-[(3R)-3-hydroxy-1-methyl-2-oxo-pyrrolidin-3-yl]ethynyl]phenyl]-6-methyl-pyrimidine-4-carboxylate was reacted to give 25 mg of the title compound (18%). M+H=351.0; 1H NMR (400 MHz, DMSO-d6) δ 8.72-8.56 (m, 3H), 7.96 (s, 1H), 7.84 (s, 1H), 7.63-7.49 (m, 2H), 6.51 (s, 1H), 3.40-3.34 (m, 2H), 2.81 (s, 3H), 2.65 (s, 3H), 2.48-2.42 (m, 1H), 2.26-2.15 (m, 1H).

### Example O

### Synthesis of 6-amino-2-[3-[2-[(3R)-3-hydroxy-1-methyl-2-oxo-pyrrolidin-3-yl]ethynyl]phenyl]pyrimidine-4-carboxamide

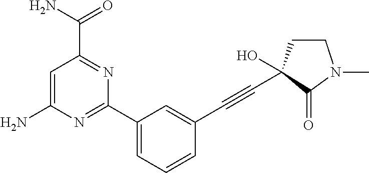 [SMILES: CN1CC[C@@](O)(C#Cc2cccc(-c3nc(N)cc(C(N)=O)n3)c2)C1=O]

Similar to as described in General Procedure W, 6-amino-2-chloro-4-pyrimidinecarboxamide was reacted with (S)-trifluoro(3-((3-hydroxy-1-methyl-2-oxopyrrolidin-3yl)ethynyl)phenyl)borate to give 58 mg of the title compound (37%). M+H=352.0; 1H NMR (400 MHz, DMSO-d6) δ 8.58-8.52 (m, 1H), 8.49 (s, 1H), 8.32 (s, 1H), 7.74 (s, 1H), 7.55-7.45 (m, 2H), 7.34 (s, 2H), 7.01-6.97 (m, 1H), 6.48 (s, 1H), 3.41-3.35 (m, 2H), 2.81 (s, 3H), 2.48-2.41 (m, 1H), 2.24-2.15 (m, 1H).

### Example P

### Synthesis of 5-fluoro-6-[3-[2-[(3R)-3-hydroxy-1-methyl-2-oxo-pyrrolidin-3-yl]ethynyl]phenyl]pyridine-2-carboxamide

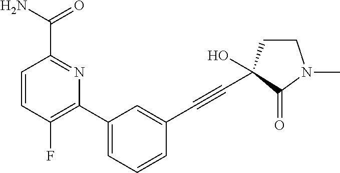 [SMILES: CN1CC[C@@](O)(C#Cc2cccc(-c3nc(C(N)=O)ccc3F)c2)C1=O]

Similar to as described in General Procedure W, 6-bromo-5-fluoro-pyridine-2-carboxamide was reacted with with (S)-trifluoro(3-((3-hydroxy-1-methyl-2-oxopyrrolidin-3yl)ethynyl)phenyl)borate to give 59 mg of the title compound (38%). M+H=354.0; 1H NMR (400 MHz, DMSO-d6) δ 8.26 (s, 1H), 8.17-8.12 (m, 1H), 8.11-8.06 (m, 2H), 8.03-7.97 (m, 1H), 7.74 (s, 1H), 7.58-7.55 (m, 2H), 6.51 (s, 1H), 3.39-3.35 (m, 2H), 2.80 (s, 3H), 2.48-2.41 (m, 1H), 2.24-2.15 (m, 1H).

### Example Q

### Synthesis of 2-[3-[2-[(3R)-3-hydroxy-1-methyl-2-oxo-pyrrolidin-3-yl]ethynyl]phenyl]-6-isopropyl-pyrimidine-4-carboxamide

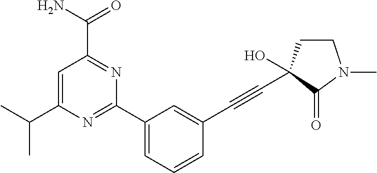 [SMILES: CC(C)c1cc(C(N)=O)nc(-c2cccc(C#C[C@]3(O)CCN(C)C3=O)c2)n1]

Similar to as described in General Procedure U, 2-chloro-6-isopropyl-pyrimidine-4-carboxylic acid methyl ester (95 mg) was reacted with (S)-trifluoro(3-((3-hydroxy-1-methyl-2-oxopyrrolidin-3yl)ethynyl)phenyl)borate to give ethyl 2-[3-[2-[(3R)-3-hydroxy-1-methyl-2-oxo-pyrrolidin-3-yl]ethynyl]phenyl]-6-isopropyl-pyrimidine-4-carboxylate. This crude material was taken on without further purification. Similar to as described in General Procedure I, ethyl 2-[3-[2-[(3R)-3-hydroxy-1-methyl-2-oxo-pyrrolidin-3-yl]ethynyl]phenyl]-6-isopropyl-pyrimidine-4-carboxylate was reacted to give 55 mg of the title compound (33%). M+H=379.0; 1H NMR (400 MHz, DMSO-d6) δ 8.73-8.60 (m, 3H), 7.98 (s, 1H), 7.85 (s, 1H), 7.63-7.52 (m, 2H), 6.52 (s, 1H), 3.40-3.34 (m, 2H), 3.24-3.15 (m, 1H), 2.81 (s, 3H), 2.50-2.43 (m, 1H), 2.28-2.15 (m, 1H), 1.34 (d, J=6.9 Hz, 6H).

### Example R

### Synthesis of 2-[3-[2-[(3R)-3-hydroxy-1-methyl-2-oxo-pyrrolidin-3-yl]ethynyl]phenyl]-6-methoxy-pyrimidine-4-carboxamide

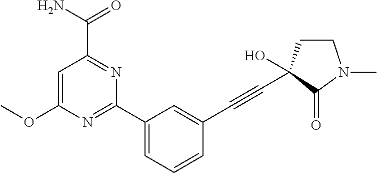

To a solution of methyl 2,6-dichloropyrimidine-4-carboxylate (1 g) in MeOH (0.25M) was added sodium methoxide (25 mass % in methanol, 1 eq). The reaction mixture was stirred at ambient temperature overnight, concentrated to dryness then re-suspended in MeOH and filtered to collect methyl 2-chloro-6-methoxy-pyrimidine-4-carboxylate (925 mg) as a white solid. Similar to as described in General Procedure U, methyl 2-chloro-6-methoxy-pyrimidine-4-carboxylate (100 mg) was reacted with (S)-trifluoro(3-((3-hydroxy-1-methyl-2-oxopyrrolidin-3yl)ethynyl)phenyl)borate to give ethyl 2-[3-[2-[(3R)-3-hydroxy-1-methyl-2-oxo-pyrrolidin-3-yl]ethynyl]phenyl]-6-methoxy-pyrimidine-4-carboxylate. This crude material was taken on without further purification. Similar to as described in General Procedure I, ethyl 2-[3-[2-[(3R)-3-hydroxy-1-methyl-2-oxo-pyrrolidin-3-yl]ethynyl]phenyl]-6-methoxy-pyrimidine-4-carboxylate was reacted to give 41 mg of the title compound (23%). M+H=367.0; 1H NMR (400 MHz, DMSO-d6) δ 8.64 (dt, J=7.6, 1.7 Hz, 1H), 8.61-8.58 (m, 1H), 8.55 (s, 1H), 7.94 (s, 1H), 7.65-7.51 (m, 2H), 7.29 (s, 1H), 6.47 (s, 1H), 4.11 (s, 3H), 3.40-3.34 (m, 2H), 2.81 (s, 3H), 2.49-2.43 (m, 1H), 2.26-2.15 (m, 1H).

### Example S

### Synthesis of 2-[3-[2-[(3R)-3-hydroxy-1-methyl-2-oxo-pyrrolidin-3-yl]ethynyl]phenyl]-6-(trifluoromethyl)pyrimidine-4-carboxamide

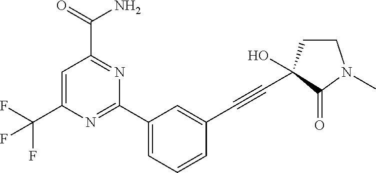 [SMILES: CN1CC[C@@](O)(C#Cc2cccc(-c3nc(C(N)=O)cc(C(F)(F)F)n3)c2)C1=O]

To a solution of 3-bromobenzamidine hydrochloride (100 mg) and ethyl 5,5,5-trifluoro-2,4-dioxo-pentanoate (1 eq) in Ethanol (0.25M) was added Sodium Ethoxide (21 mass % in Ethanol, 4 eq). The reaction mixture was stirred at 80° C. for 18 hours. The reaction mixture was quenched with 20 mL of water and the aqueous solution was acidified to pH 3. The resultant solid is collected by filtration to give 37 mg of 2-(3-bromophenyl)-6-(trifluoromethyl)pyrimidine-4-carboxylic acid. This crude material was taken on without further purification. Similar to as described in General Procedure B, 2-(3-bromophenyl)-6-(trifluoromethyl)pyrimidine-4-carboxylic acid was reacted with ammonium chloride to give 37 mg of 2-(3-bromophenyl)-6-(trifluoromethyl)pyrimidine-4-carboxamide. This crude material was taken on without further purification. Similar to as described in General Procedure E, of 2-(3-bromophenyl)-6-(trifluoromethyl)pyrimidine-4-carboxamide (37 mg) was reacted with (3R)-3-ethynyl-3-hydroxy-1-methyl-pyrrolidin-2-one to give 4 mg of the title compound (8%). M+H=405.0

### Example T

### Synthesis of 2-[3-[2-[(3R)-3-hydroxy-1-methyl-2-oxo-pyrrolidin-3-yl]ethynyl]phenyl]-6-[2-(methoxymethyl)pyrrolidin-1-yl]pyrimidine-4-carboxamide

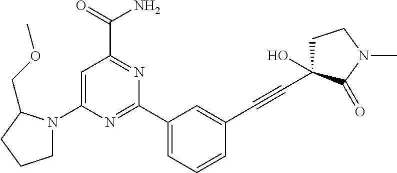 [SMILES: COCC1CCCN1c1cc(C(N)=O)nc(-c2cccc(C#C[C@]3(O)CCN(C)C3=O)c2)n1]

Similar to as described in General Procedure A, 2,6-dichloropyrimidine-4-carboxamide (100 mg) was reacted with 2-(methoxymethyl)pyrrolidine to give 2-chloro-6-[2-(methoxymethyl)pyrrolidin-1-yl]pyrimidine-4-carboxamide. This intermediate was taken onto the next step without purification. Similar to as described in General Procedure W, 2-chloro-6-[2-(methoxymethyl)pyrrolidin-1-yl]pyrimidine-4-carboxamide was then reacted with (S)-trifluoro(3-((3-hydroxy-1-methyl-2-oxopyrrolidin-3yl)ethynyl)phenyl)borate to give 72 mg of the title compound (31%). 1H NMR (400 MHz, DMSO-d6) δ 8.57 (d, J=7.4 Hz, 1H), 8.53 (s, 1H), 8.40 (s, 1H), 7.82-7.77 (m, 1H), 7.58-7.48 (m, 2H), 7.04 (d, J=69.3 Hz, 1H), 6.48 (s, 1H), 4.62-4.06 (m, 2H), 3.77-3.51 (m, 2H), 3.37 (s, 3H), 2.81 (s, 3H), 2.48-2.39 (m, 1H), 2.24-2.15 (m, 1H), 2.11-1.92 (m, 4H).

### Example U

### Synthesis of 2-[3-[2-[(3R)-3-hydroxy-1-methyl-2-oxo-pyrrolidin-3-yl]ethynyl]phenyl]-6-[methoxy(methyl)amino]pyrimidine-4-carboxamide

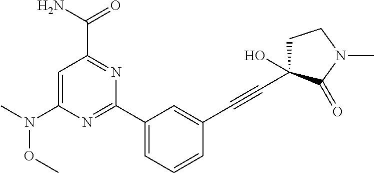

Similar to as described in General Procedure A, 2,6-dichloropyrimidine-4-carboxamide (80 mg) was reacted with N,O-dimethylhydroxylamine hydrochloride to give 2-chloro-6-(methoxy(methyl)amino)pyrimidine-4-carboxamide. This intermediate was taken onto the next step without purification. Similar to as described in General Procedure W, 2-chloro-6-(methoxy(methyl)amino)pyrimidine-4-carboxamide was then reacted with (S)-trifluoro(3-((3-hydroxy-1-methyl-2-oxopyrrolidin-3yl)ethynyl)phenyl)borate to give 59 mg of the title compound (36%). M+H=396.0; 1H NMR (400 MHz, DMSO-d6) δ 8.57 (dt, J=7.5, 1.6 Hz, 1H), 8.53-8.51 (m, 1H), 8.46 (s, 1H), 7.85 (s, 1H), 7.59-7.50 (m, 2H), 7.37 (s, 1H), 6.45 (s, 1H), 3.81 (s, 3H), 3.46 (s, 3H), 3.39-3.34 (m, 2H), 2.81 (s, 3H), 2.48-2.43 (m, 1H), 2.24-2.16 (m, 1H).

### Example V

### Synthesis of 2-[3-[2-[(3R)-3-hydroxy-1-methyl-2-oxo-pyrrolidin-3-yl]ethynyl]phenyl]-6-indolin-1-yl-pyrimidine-4-carboxamide

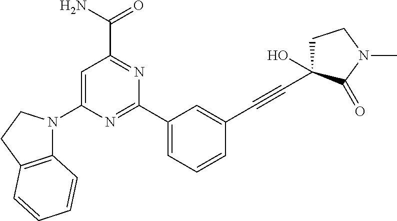 [SMILES: CN1CC[C@@](O)(C#Cc2cccc(-c3nc(C(N)=O)cc(N4CCc5ccccc54)n3)c2)C1=O]

Methyl 2,4-dichloropyrimidine-6-carboxylate (150 mg) was reacted with a pre-stirred (0.5 hr) mixture of indoline (1 eq) and Sodium Hydride (1.1 eq) in DMF (0.1 M) to give methyl 2-chloro-6-indolin-1-yl-pyrimidine-4-carboxylate as a brown semi-solid following standard extractive workup with DCM and Saturated Ammonium Chloride. This intermediate was taken onto the next step without purification. Similar to as described in General Procedure W, methyl 2-chloro-6-indolin-1-yl-pyrimidine-4-carboxylate was reacted with (S)-trifluoro(3-((3-hydroxy-1-methyl-2-oxopyrrolidin-3yl)ethynyl)phenyl)borate to give ethyl 2-[3-[2-[(3R)-3-hydroxy-1-methyl-2-oxo-pyrrolidin-3-yl]ethynyl]phenyl]-6-indolin-1-yl-pyrimidine-4-carboxylate. This intermediate was taken onto the next step without purification. Similar to as described in General Procedure I, ethyl 2-[3-[2-[(3R)-3-hydroxy-1-methyl-2-oxo-pyrrolidin-3-yl]ethynyl]phenyl]-6-indolin-1-yl-pyrimidine-4-carboxylate was reacted to afford 4.1 mg of the title compound (3%). M+H=454.0

### Example W

### Synthesis of 2-[3-[2-[(3R)-3-hydroxy-1-methyl-2-oxo-pyrrolidin-3-yl]ethynyl]phenyl]-6-(2-oxa-8-azaspiro[3.4]octan-8-yl)pyrimidine-4-carboxamide

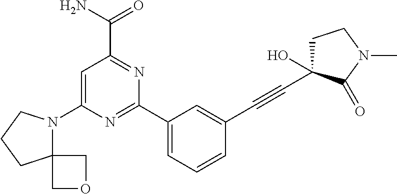 [SMILES: CN1CC[C@@](O)(C#Cc2cccc(-c3nc(C(N)=O)cc(N4CCCC45COC5)n3)c2)C1=O]

Similar to as described in General Procedure A, 2,6-dichloropyrimidine-4-carboxamide (80 mg) was reacted with 2-oxa-5-azaspiro[3.4]octane oxalate to give 2-chloro-6-(2-oxa-8-azaspiro[3.4]octan-8-yl)pyrimidine-4-carboxamide. This intermediate was taken onto the next step without purification. Similar to as described in General Procedure W, 2-chloro-6-(2-oxa-8-azaspiro[3.4]octan-8-yl)pyrimidine-4-carboxamide was then reacted with (S)-trifluoro(3-((3-hydroxy-1-methyl-2-oxopyrrolidin-3yl)ethynyl)phenyl)borate to give 7 mg of the title compound (4%). M+H=448.0; 1H NMR (400 MHz, DMSO-d6) δ 8.78-8.65 (m, 2H), 8.43 (s, 1H), 7.79 (s, 1H), 7.60-7.46 (m, 2H), 7.24-6.29 (m, 1H), 5.66 (s, 1H), 4.47 (s, 2H), 3.53 (s, 2H), 3.42-3.33 (m, 4H), 2.80 (s, 3H), 2.47-2.39 (m, 3H), 2.24-2.14 (m, 1H), 1.94-1.82 (m, 2H).

### Example X and Example Y

### Synthesis of 2-[3-[2-[(3R)-3-hydroxy-1-methyl-2-oxo-pyrrolidin-3-yl]ethynyl]phenyl]-6-[(2R)-2-methylazetidin-1-yl]pyrimidine-4-carboxamide AND 2-[3-[2-[(3R)-3-hydroxy-1-methyl-2-oxo-pyrrolidin-3-yl]ethynyl]phenyl]-6-[(2 S)-2-methylazetidin-1-yl]pyrimidine-4-carboxamide

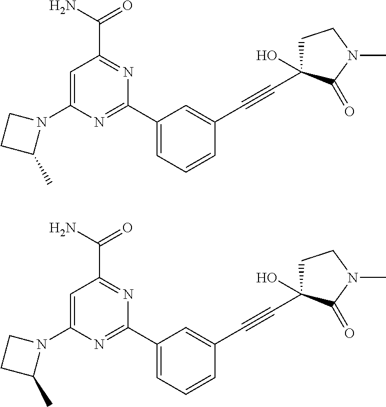 [SMILES: C[C@@H]1CCN1c1cc(C(N)=O)nc(-c2cccc(C#C[C@]3(O)CCN(C)C3=O)c2)n1 ; C[C@H]1CCN1c1cc(C(N)=O)nc(-c2cccc(C#C[C@]3(O)CCN(C)C3=O)c2)n1]

Similar to as described in General Procedure A, 2,6-dichloropyrimidine-4-carboxamide (80 mg) was reacted with 2-methylazetidine hydrochloride to give 2-chloro-6-(2-methylazetidin-1-yl)pyrimidine-4-carboxamide. This intermediate was taken onto the next step without purification. Similar to as described in General Procedure W, 2-chloro-6-(2-methylazetidin-1-yl)pyrimidine-4-carboxamide was reacted with (S)-trifluoro(3-((3-hydroxy-1-methyl-2-oxopyrrolidin-3yl)ethynyl)phenyl)borate to give 21 mg and 18 mg (22% overall yield), respectively, following reverse phase and chiral purifications.

M+H=406.0; 1H NMR (400 MHz, DMSO-d6) δ 8.52 (dt, J=7.5, 1.7 Hz, 1H), 8.50-8.47 (m, 1H), 8.35 (s, 1H), 7.76 (s, 1H), 7.55-7.47 (m, 2H), 6.79 (s, 1H), 6.45 (s, 1H), 4.67-4.55 (m, 1H), 4.20-4.09 (m, 1H), 4.08-3.97 (m, 1H), 3.40-3.33 (m, 2H), 2.81 (s, 3H), 2.61-2.53 (m, 1H), 2.47-2.42 (m, 1H), 2.25-2.14 (m, 1H), 2.09-1.99 (m, 1H), 1.55 (d, J=6.1 Hz, 3H).

M+H=406.0; 1H NMR (400 MHz, DMSO-d6) δ 8.52 (dt, J=7.5, 1.7 Hz, 1H), 8.49-8.47 (m, 1H), 8.35 (s, 1H), 7.76 (s, 1H), 7.56-7.48 (m, 2H), 6.79 (s, 1H), 6.45 (s, 1H), 4.66-4.57 (m, 1H), 4.19-4.11 (m, 1H), 4.07-3.97 (m, 1H), 3.40-3.33 (m, 2H), 2.81 (s, 3H), 2.60-2.52 (m, 1H), 2.47-2.42 (m, 1H), 2.25-2.16 (m, 1H), 2.09-1.99 (m, 1H), 1.55 (d, J=6.0 Hz, 3H).

### Example Z

### Synthesis of 2-[3-[2-[(3R)-3-hydroxy-1-methyl-2-oxo-pyrrolidin-3-yl]ethynyl]phenyl]-6-[(3R)-3-hydroxypyrrolidin-1-yl]pyrimidine-4-carboxamide

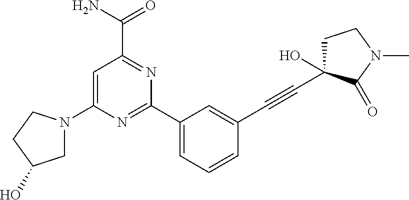 [SMILES: CN1CC[C@@](O)(C#Cc2cccc(-c3nc(C(N)=O)cc(N4CC[C@@H](O)C4)n3)c2)C1=O]

Similar to as described in General Procedure A, 2,6-dichloropyrimidine-4-carboxamide (80 mg) was reacted with 3-pyrrolidinol to give 2-chloro-6-(3-hydroxypyrrolidin-1-yl)pyrimidine-4-carboxamide. This intermediate was taken onto the next step without purification. Similar to as described in General Procedure W, 2-chloro-6-(3-hydroxypyrrolidin-1-yl)pyrimidine-4-carboxamide was then reacted with (S)-trifluoro(3-((3-hydroxy-1-methyl-2-oxopyrrolidin-3yl)ethynyl)phenyl)borate to give 7 mg of the title compound (4%). M+H=422.0; 1H NMR (400 MHz, DMSO-d6) δ 8.58 (dt, J=7.5, 1.7 Hz, 1H), 8.52-8.50 (m, 1H), 8.36 (s, 1H), 7.76 (s, 1H), 7.57-7.47 (m, 2H), 7.00-6.89 (m, 1H), 6.45 (s, 1H), 5.05 (d, J=32.2 Hz, 1H), 4.51-4.40 (m, 1H), 3.91-3.51 (m, 4H), 3.41-3.34 (m, 2H), 2.81 (s, 3H), 2.48-2.43 (m, 1H), 2.26-2.15 (m, 1H), 2.14-1.89 (m, 2H).

### Example AA and Example BB

### Synthesis of 2-[3-[2-[(3R)-3-hydroxy-1-methyl-2-oxo-pyrrolidin-3-yl]ethynyl]phenyl]-6-[(2R)-2-isobutylpyrrolidin-1-yl]pyrimidine-4-carboxamide AND 2-[3-[2-[(3R)-3-hydroxy-1-methyl-2-oxo-pyrrolidin-3-yl]ethynyl]phenyl]-6-[(2 S)-2-isobutylpyrrolidin-1-yl]pyrimidine-4-carboxamide

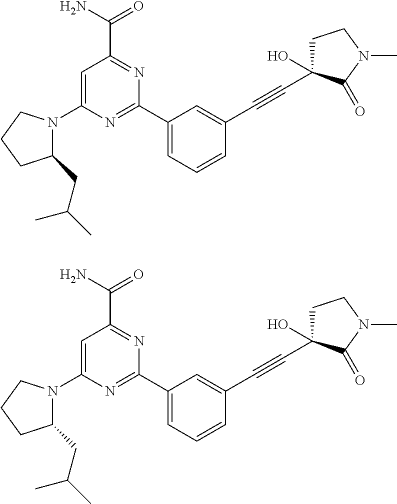

Similar to as described in General Procedure A, 2,6-dichloropyrimidine-4-carboxamide (80 mg) was reacted with 2-isobutylpyrrolidine to give (R)-2-chloro-6-(2-isobutylpyrrolidin-1-yl)pyrimidine-4-carboxamide. This intermediate was taken onto the next step without purification. Similar to as described in General Procedure W, (R)-2-chloro-6-(2-isobutylpyrrolidin-1-yl)pyrimidine-4-carboxamide was reacted with (S)-trifluoro(3-((3-hydroxy-1-methyl-2-oxopyrrolidin-3yl)ethynyl)phenyl)borate to give 13 mg and 12 mg (13% overall yield) following reverse phase and chiral purifications.

M+H=462.0; 1H NMR (400 MHz, DMSO-d6) δ 8.63-8.50 (m, 2H), 8.34 (s, 1H), 7.74 (s, 1H), 7.57-7.46 (m, 2H), 6.95 (s, 1H), 6.42 (s, 1H), 4.55-4.38 (m, 1H), 4.10-3.62 (m, 1H), 3.59-3.47 (m, 1H), 3.39-3.32 (m, 2H), 2.81 (s, 3H), 2.47-2.38 (m, 1H), 2.24-2.15 (m, 1H), 2.12-1.92 (m, 3H), 1.90-1.83 (m, 1H), 1.80-1.61 (m, 2H), 1.44-1.21 (m, 1H), 1.15-1.03 (m, 3H), 0.94 (d, J=6.3 Hz, 3H).

M+H=462.0; 1H NMR (400 MHz, DMSO-d6) δ 8.65-8.47 (m, 2H), 8.34 (s, 1H), 7.74 (s, 1H), 7.58-7.42 (m, 2H), 6.92 (s, 1H), 6.42 (s, 1H), 4.55-4.41 (m, 1H), 4.09-3.61 (m, 1H), 3.58-3.48 (m, 1H), 3.39-3.33 (m, 2H), 2.81 (s, 3H), 2.48-2.39 (m, 1H), 2.24-2.15 (m, 1H), 2.11-1.94 (m, 3H), 1.91-1.83 (m, 1H), 1.80-1.65 (m, 2H), 1.45-1.18 (m, 1H), 1.14-1.02 (m, 3H), 0.95 (d, J=6.2 Hz, 3H).

### Example CC and Example DD

### Synthesis of 2-[3-[2-[(3R)-3-hydroxy-1-methyl-2-oxo-pyrrolidin-3-yl]ethynyl]phenyl]-6-[[(3 S)-tetrahydrofuran-3-yl]amino]pyrimidine-4-carboxamide AND 2-[3-[2-[(3R)-3-hydroxy-1-methyl-2-oxo-pyrrolidin-3-yl]ethynyl]phenyl]-6-[[(3R)-tetrahydrofuran-3-yl]amino]pyrimidine-4-carboxamide

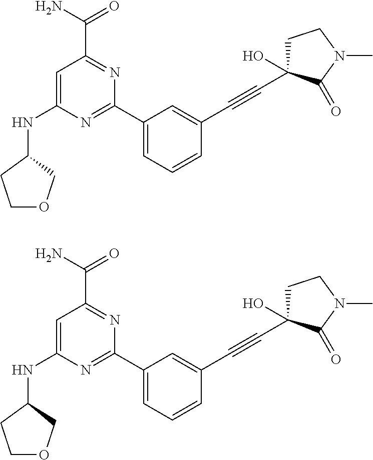 [SMILES: CN1CC[C@@](O)(C#Cc2cccc(-c3nc(N[C@H]4CCOC4)cc(C(N)=O)n3)c2)C1=O ; CN1CC[C@@](O)(C#Cc2cccc(-c3nc(N[C@@H]4CCOC4)cc(C(N)=O)n3)c2)C1=O]

Similar to as described in General Procedure A, 2,6-dichloropyrimidine-4-carboxamide (80 mg) was reacted with 3-aminotetrahydrofuran to give 2-chloro-6-(tetrahydrofuran-3-ylamino)pyrimidine-4-carboxamide. This intermediate was taken onto the next step without purification. Similar to as described in General Procedure W, 2-chloro-6-(tetrahydrofuran-3-ylamino)pyrimidine-4-carboxamide was reacted with (S)-trifluoro(3-((3-hydroxy-1-methyl-2-oxopyrrolidin-3yl)ethynyl)phenyl)borate to give 3 mg and 10 mg (8% overall yield) of the title compounds. M+H=422.0; 1H NMR (400 MHz, DMSO-d6) δ 8.54 (dt, J=7.4, 1.7 Hz, 1H), 8.51-8.48 (m, 1H), 8.30 (s, 1H), 8.07 (d, J=6.3 Hz, 1H), 7.71 (s, 1H), 7.56-7.47 (m, 2H), 7.05 (s, 1H), 6.45 (s, 1H), 4.74-4.60 (m, 1H), 4.02-3.94 (m, 1H), 3.92-3.83 (m, 1H), 3.81-3.74 (m, 1H), 3.65-3.59 (m, 1H), 3.40-3.34 (m, 2H), 2.81 (s, 3H), 2.48-2.40 (m, 1H), 2.35-2.25 (m, 1H), 2.24-2.15 (m, 1H), 1.94-1.84 (m, 1H).

### Synthesis of 2-chloro-6-(2-methylpyrazol-3-yl)pyrimidine-4-carboxamide

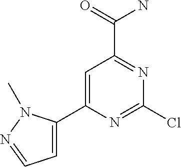 [SMILES: Cn1nccc1-c1cc(C(N)=O)nc(Cl)n1]

Similar to as described in General Procedure M, methyl 2,6-dichloropyrimidine-4-carboxylate (100 mg) was reacted with 1-methyl-5-(4,4,5,5-tetramethyl-1,3,2-dioxaborolan-2-yl)pyrazole (1 eq) and tetrakis(triphenylphosphine)palladium(0) (0.1 eq) in a solution of Dimethoxyethane (0.25M) and 1M Sodium Carbonate (3 eq) for 18 hours at 100° C. overnight. The solution was allowed to cool to room temperature, the DME was removed under vacuum and the aqueous solution was then acidified to pH 3. The resultant solid was collected by filtration and dried under vacuum overnight to give 2-chloro-6-(2-methylpyrazol-3-yl)pyrimidine-4-carboxylic acid. This intermediate was taken on without purification.

Similar to as described in General Procedure B, 2-chloro-6-(2-methylpyrazol-3-yl)pyrimidine-4-carboxylic acid was reacted with ammonium chloride to give 110 mg (96%) of 2-chloro-6-(2-methylpyrazol-3-yl)pyrimidine-4-carboxamide following trituration from saturated ammonium chloride.

### Example EE

### Synthesis of 2-[3-[2-[(3R)-3-hydroxy-1-methyl-2-oxo-pyrrolidin-3-yl]ethynyl]phenyl]-6-(2-methylpyrazol-3-yl)pyrimidine-4-carboxamide

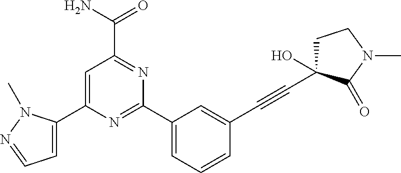 [SMILES: CN1CC[C@@](O)(C#Cc2cccc(-c3nc(C(N)=O)cc(-c4ccnn4C)n3)c2)C1=O]

Similar to as described in General Procedure W, 2-chloro-6-(2-methylpyrazol-3-yl)pyrimidine-4-carboxamide (110 mg) was reacted with (S)-trifluoro(3-((3-hydroxy-1-methyl-2-oxopyrrolidin-3yl)ethynyl)phenyl)borate to give 6 mg (4%) of the title compound. M+H=417.0; 1H NMR (400 MHz, DMSO-d6) δ 8.72 (s, 1H), 8.70-8.64 (m, 2H), 8.24 (s, 1H), 8.03 (s, 1H), 7.67-7.56 (m, 3H), 7.30 (d, J=2.0 Hz, 1H), 6.47 (s, 1H), 4.38 (s, 3H), 3.41-3.35 (m, 2H), 2.82 (s, 3H), 2.48-2.43 (m, 1H), 2.26-2.16 (m, 1H).

### Example FF

### Synthesis of 6-(3-[2-[(3R)-3-hydroxy-1-methyl-2-oxopyrrolidin-3-yl]ethynyl]phenyl)-1-methyl-1H-pyrazolo[3,4-d]pyrimidine-4-carboxamide

### Step 1: Synthesis of 4,6-dichloro-1-methyl-1H-pyrazolo[3,4-d]pyrimidine

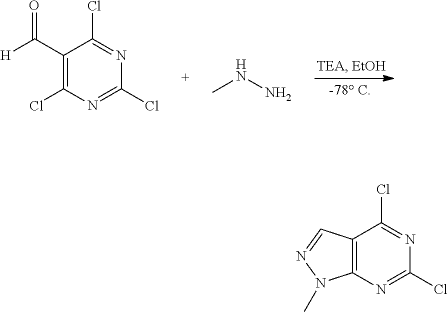

To a solution of 2,4,6-trichloropyrimidine-5-carbaldehyde (8 g, 37.84 mmol, 1.00 equiv) in ethanol (120 mL) was added a 40% water solution of methylhydrazine (4 mL, 37.98 mmol, 1.00 equiv) and triethylamine (16 mL) at −78° C. The resulting mixture was stirred for 30 min at −78° C. and then 2 h at 0° C. After completion the reaction was concentrated under vacuum without heating. Then ethyl acetate was added and the solution was washed with saturated ammonium chloride solution. The organic layer was dried over anhydrous sodium sulfate and concentrated under vacuum without heating. The residue was purified by silica gel column chromatography eluting with ethyl acetate/petroleum ether (1:1) to afford 6 g (78%) of 4,6-dichloro-1-methyl-1H-pyrazolo[3,4-d]pyrimidine as a white solid. LC-MS (ES, m/z): 203 [M+H]⁺.

### Step 2: Synthesis of 6-chloro-4-(1-ethoxyethenyl)-1-methyl-1H-pyrazolo[3,4-d]pyrimidine

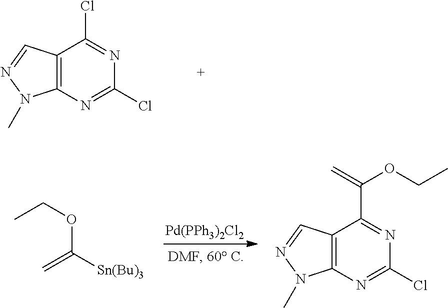

Similar to as described in General Procedure Q, 4,6-dichloro-1-methyl-1H-pyrazolo[3,4-d]pyrimidine was reacted with tributyl(1-ethoxyethenyl)stannane to give the title compound (1.3 g, 55%) as a white solid. LC-MS (ES, m/z): 239 [M+H]⁺.

### Step 3: Synthesis of ethyl 6-chloro-1-methyl-1H-pyrazolo[3,4-d]pyrimidine-4-carboxylate

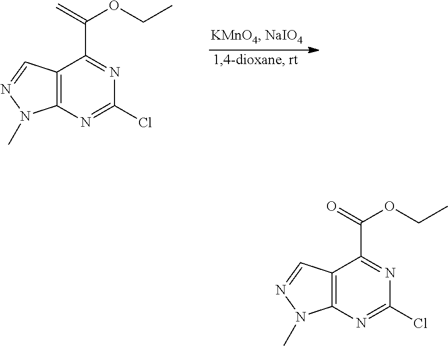 [SMILES: C=C(OCC)c1nc(Cl)nc2c1cnn2C ; CCOC(=O)c1nc(Cl)nc2c1cnn2C]

Similar to as described in General Procedure R, 6-chloro-4-(1-ethoxyethenyl)-1-methyl-1H-pyrazolo[3,4-d]pyrimidine was reacted with potassium permanganate to give the title compound (600 mg, 46%) as a white solid. LC-MS (ES, m/z): 241 [M+H]⁺.

### Step 4: Synthesis of ethyl 6-(3-bromophenyl)-1-methyl-1H-pyrazolo[3,4-d]pyrimidine-4-carboxylate

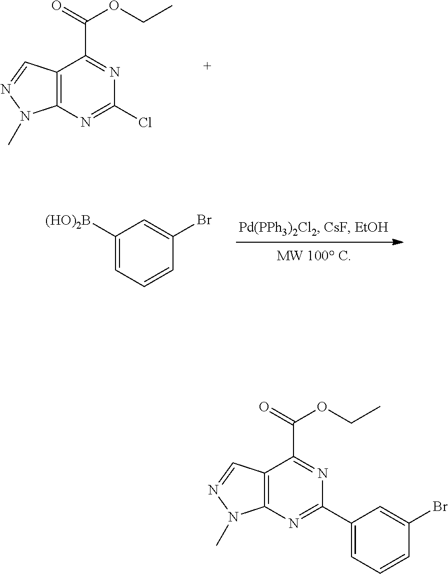 [SMILES: CCOC(=O)c1nc(Cl)nc2c1cnn2C ; OB(O)c1cccc(Br)c1 ; CCOC(=O)c1nc(-c2cccc(Br)c2)nc2c1cnn2C]

Similar to as described in General Procedure M, ethyl 6-chloro-1-methyl-1H-pyrazolo[3,4-d]pyrimidine-4-carboxylate was reacted with (3-bromophenyl)boronic acid to give the title compound (180 mg, 40%) as a white solid. LC-MS (ES, m/z): 361, 363 [M+H]⁺.

### Step 5: Synthesis of ethyl 6-(3-[2-[(3R)-3-hydroxy-1-methyl-2-oxopyrrolidin-3-yl]ethynyl]phenyl)-1-methyl-1H-pyrazolo[3,4-d]pyrimidine-4-carboxylate

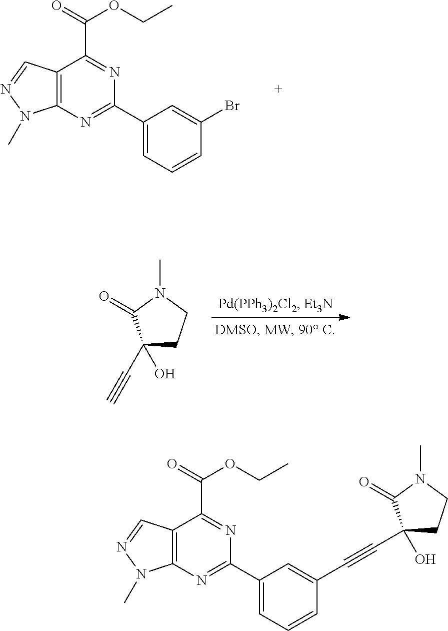

Similar to as described in General Procedure E, ethyl 6-(3-bromophenyl)-1-methyl-1H-pyrazolo[3,4-d]pyrimidine-4-carboxylate was reacted with (3R)-3-ethynyl-3-hydroxy-1-methylpyrrolidin-2-one to give the title compound (160 mg, 92%) as a yellow solid. LC-MS (ES, m/z): 420 [M+H]⁺.

### Step 6: Synthesis of 6-(3-[2-[(3R)-3-hydroxy-1-methyl-2-oxopyrrolidin-3-yl]ethynyl]phenyl)-1-methyl-1H-pyrazolo[3,4-d]pyrimidine-4-carboxamide

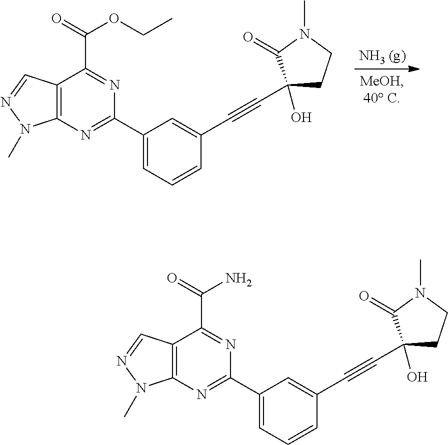 [SMILES: CCOC(=O)c1nc(-c2cccc(C#C[C@]3(O)CCN(C)C3=O)c2)nc2c1cnn2C ; CN1CC[C@@](O)(C#Cc2cccc(-c3nc(C(N)=O)c4cnn(C)c4n3)c2)C1=O]

Similar to as described in General Procedure S, ethyl 6-(3-[2-[(3R)-3-hydroxy-1-methyl-2-oxopyrrolidin-3-yl]ethynyl]phenyl)-1-methyl-1H-pyrazolo[3,4-d]pyrimidine-4-carboxylate was reacted with ammonia in methanol to give the title compound (80 mg, 54%) as an off-white solid. LC-MS (ES, m/z): 391 [M+H]⁺. ¹H NMR (400 MHz, CD₃OD) δ 8.70 (s, 1H), 8.63 (d, J=8 Hz, 1H), 8.55 (s, 1H), 7.59 (d, J=8 Hz, 1H), 7.50 (t, J=7.6 Hz, 1H), 4.17 (s, 3H), 3.58-3.49 (m, 2H), 2.98 (s, 3H), 2.69-2.63 (m, 1H), 2.41-2.33 (m, 1H).

### Example GG

### Synthesis of 3-(3-[2-[(3R)-3-hydroxy-1-methyl-2-oxopyrrolidin-3-yl]ethynyl]phenyl)isoquinoline-1-carboxamide

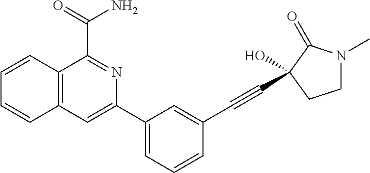 [SMILES: CN1CC[C@@](O)(C#Cc2cccc(-c3cc4ccccc4c(C(N)=O)n3)c2)C1=O]

### Step 1: Synthesis of 3-chloro-1-(1-ethoxyethenyl)isoquinoline

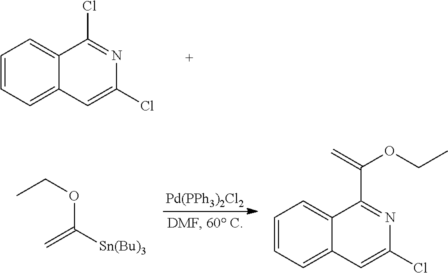

Similar to as described in General Procedure Q, 1,3-dichloroisoquinoline was reacted with tributyl(1-ethoxyethenyl)stannane to give the title compound (2 g, 56%) as a white solid. LC-MS (ES, m/z): 234 [M+H]⁺.

### Step 2: Synthesis of ethyl 3-chloroisoquinoline-1-carboxylate

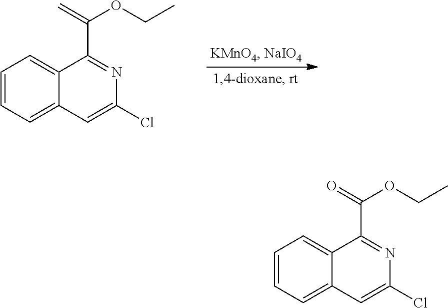 [SMILES: C=C(OCC)c1nc(Cl)cc2ccccc12 ; CCOC(=O)c1nc(Cl)cc2ccccc12]

Similar to as described in General Procedure R, 3-chloro-1-(1-ethoxyethenyl)isoquinoline was reacted with potassium permanganate to give the title compound (800 mg, 40%) as colorless oil. LC-MS (ES, m/z): 236 [M+H]⁺.

### Step 3: Synthesis of ethyl 3-(3-[2-[(3R)-3-hydroxy-1-methyl-2-oxopyrrolidin-3-yl]ethynyl]phenyl)isoquinoline-1-carboxylate

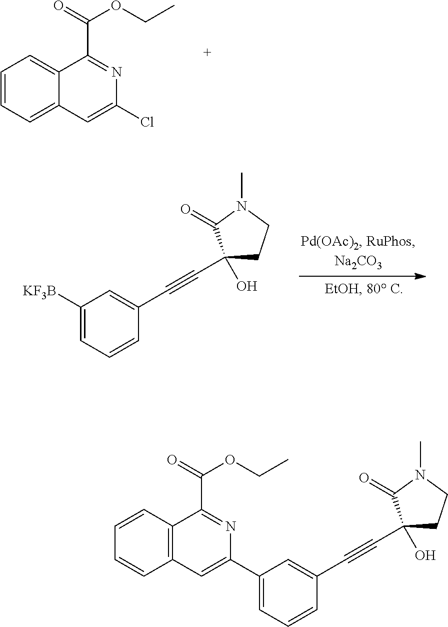

Similar to as described in General Procedure U, ethyl 3-chloroisoquinoline-1-carboxylate was reacted with potassium trifluoro(3-{2-[(3R)-3-hydroxy-1-methyl-2-oxopyrrolidin-3-yl]ethynyl}phenyl)boranuide to give the title compound (120 mg, 34%) as brown oil. LC-MS (ES, m/z): 415 [M+H]⁺.

### Step 4: Synthesis of 3-(3-[2-[(3R)-3-hydroxy-1-methyl-2-oxopyrrolidin-3-yl]ethynyl]phenyl)isoquinoline-1-carboxamide

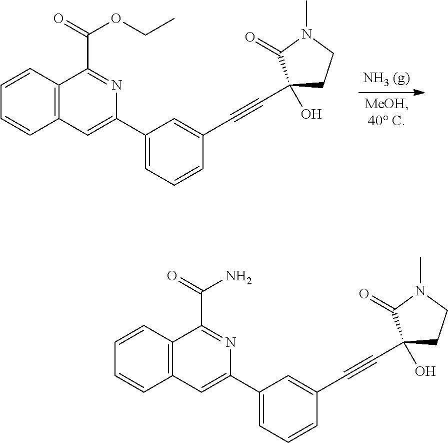 [SMILES: CCOC(=O)c1nc(-c2cccc(C#C[C@]3(O)CCN(C)C3=O)c2)cc2ccccc12 ; CN1CC[C@@](O)(C#Cc2cccc(-c3cc4ccccc4c(C(N)=O)n3)c2)C1=O]

Similar to as described in General Procedure S, ethyl 3-(3-[2-[(3R)-3-hydroxy-1-methyl-2-oxopyrrolidin-3-yl]ethynyl]phenyl)isoquinoline-1-carboxylate was reacted with ammonia in methanol to give the title compound (24.7 mg, 22%) as a white solid. LC-MS (ES, m/z): 386 [M+H]⁺. ¹H NMR (400 MHz, CDCl₃) δ 9.54 (d, J=7.6 Hz, 1H), 8.12-8.11 (m, 2H), 8.02 (d, J=7.6 Hz, 1H), 7.83 (d, J=7.6 Hz, 1H), 7.74-7.66 (m, 2H), 7.50 (d, J=Hz, 1H), 7.45-7.41 (m, 1H), 5.98 (s, 1H), 3.78 (s, 1H), 3.56-3.50 (m, 1H), 3.44-3.39 (m, 1H), 2.99 (s, 3H), 2.72-2.67 (m, 1H), 2.45-2.40 (m, 1H).

### Example HH

### Synthesis of 2-(3-[2-[(3R)-3-hydroxy-1-methyl-2-oxopyrrolidin-3-yl]ethynyl]phenyl)-7-methyl-7H-pyrrolo[2,3-d]pyrimidine-4-carboxamide

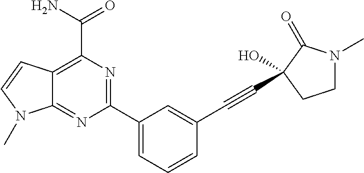 [SMILES: CN1CC[C@@](O)(C#Cc2cccc(-c3nc(C(N)=O)c4ccn(C)c4n3)c2)C1=O]

### Step 1: Synthesis of 2,4-dichloro-7-methyl-7H-pyrrolo[2,3-d]pyrimidine

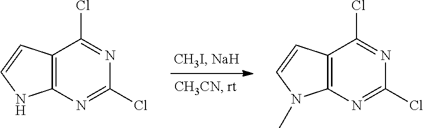

To a solution of sodium hydride (480 mg, 12.00 mmol, 1.10 equiv, 60%) in tetrahydrofuran (50 mL), a solution of 2,4-di chloro-7H-pyrrolo[2,3-d]pyrimidine (2 g, 10.64 mmol, 1.00 equiv) in tetrahydrofuran (50 mL) was slowly added. The resulting solution was stirred at 0° C. for 30 min followed by the addition of methyl iodide (1.66 g, 11.70 mmol, 1.10 equiv) at 0° C. The mixture was stirred at room temperature overnight. After completion, 20 mL of water was added to the mixture and the solution was extracted with ethyl acetate and washed with brine. The organic phase was dried over anhydrous sodium sulfate, filtered and then concentrated under vacuum. The residue was purified by silica gel column chromatography eluting with ethyl acetate/petroleum ether (1:5) to afford 2.1 g (98%) of 2,4-dichloro-7-methyl-7H-pyrrolo[2,3-d]pyrimidine as a white solid. LC-MS (ES, m/z): 202 [M+H]⁺.

### Step 2: Synthesis of 2-chloro-4-(1-ethoxyethenyl)-7-methyl-7H-pyrrolo[2,3-d]pyrimidine

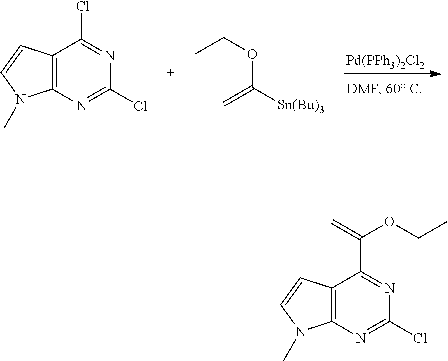

Similar to as described in General Procedure Q, 2,4-dichloro-7-methyl-7H-pyrrolo[2,3-d]pyrimidine was reacted with tributyl(1-ethoxyethenyl)stannane to give the title compound (1.75 g, 71%) as a yellow solid. LC-MS (ES, m/z): 238 [M+H]⁺.

### Step 3: Synthesis of ethyl 2-chloro-7-methyl-7H-pyrrolo[2,3-d]pyrimidine-4-carboxylate

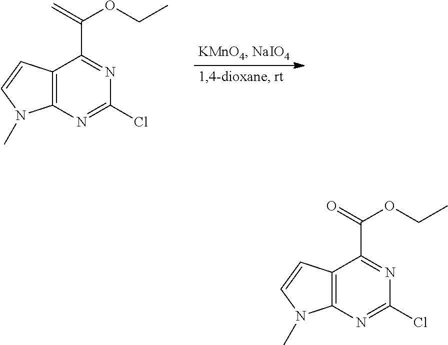 [SMILES: C=C(OCC)c1nc(Cl)nc2c1ccn2C ; CCOC(=O)c1nc(Cl)nc2c1ccn2C]

Similar to as described in General Procedure R, 2-chloro-4-(1-ethoxyethenyl)-7-methyl-7H-pyrrolo[2,3-d]pyrimidine was reacted with potassium permanganate to give the title compound (800 mg, 45%) as a yellow solid. LC-MS (ES, m/z): 240 [M+H]⁺.

### Step 4: Synthesis of ethyl 2-(3-[2-[(3R)-3-hydroxy-1-methyl-2-oxopyrrolidin-3-yl]ethynyl]phenyl)-7-methyl-7H-pyrrolo[2,3-d]pyrimidine-4-carboxylate

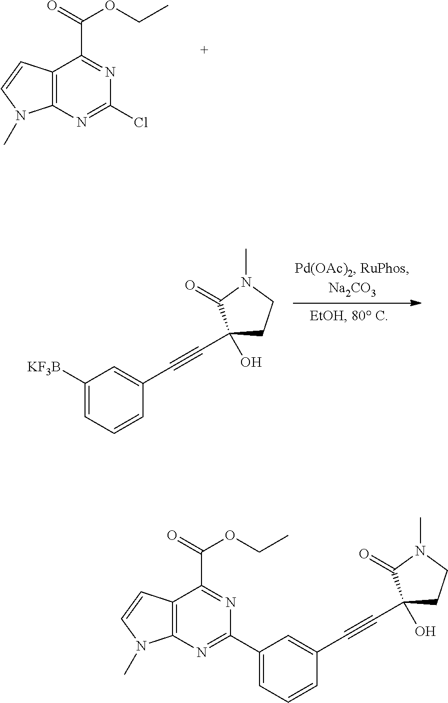

Similar to as described in General Procedure U, ethyl 2-chloro-7-methyl-7H-pyrrolo[2,3-d]pyrimidine-4-carboxylate was reacted with potassium trifluoro(3-{2-[(3R)-3-hydroxy-1-methyl-2-oxopyrrolidin-3-yl]ethynyl}phenyl)boranuide to give the title compound (100 mg, 29%) as a yellow solid. LC-MS (ES, m/z): 419 [M+H]⁺.

### Step 5: Synthesis of 2-(3-[2-[(3R)-3-hydroxy-1-methyl-2-oxopyrrolidin-3-yl]ethynyl]phenyl)-7-methyl-7H-pyrrolo[2,3-d]pyrimidine-4-carboxamide

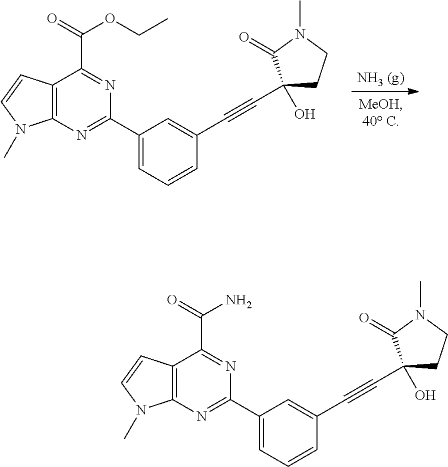 [SMILES: CCOC(=O)c1nc(-c2cccc(C#C[C@]3(O)CCN(C)C3=O)c2)nc2c1ccn2C ; CN1CC[C@@](O)(C#Cc2cccc(-c3nc(C(N)=O)c4ccn(C)c4n3)c2)C1=O]

Similar to as described in General Procedure S, ethyl 2-(3-[2-[(3R)-3-hydroxy-1-methyl-2-oxopyrrolidin-3-yl]ethynyl]phenyl)-7-methyl-7H-pyrrolo[2,3-d]pyrimidine-4-carboxylate was reacted with ammonia in methanol to give the title compound (65 mg, 70%) as a white solid. LC-MS (ES, m/z): 390 [M+H]⁺. ¹H NMR (400 MHz, CDCl₃) δ 8.55 (s, 1H), 8.45 (d, J=7.6 Hz, 1H), 8.11 (s, 1H), 7.50 (d, J=7.6 Hz, 1H), 7.41-7.38 (m, 1H), 7.33-7.31 (m, 1H), 6.14 (s, 1H), 3.95 (s, 3H), 3.56-3.50 (m, 1H), 3.44-3.38 (m, 1H), 2.98 (s, 3H), 2.72-2.67 (m, 1H), 2.46-2.38 (m, 1H).

### Example II

### Synthesis of 2-(3-[2-[(3R)-3-hydroxy-1-methyl-2-oxopyrrolidin-3-yl]ethynyl]phenyl)thieno[3,2-d]pyrimidine-4-carboxamide

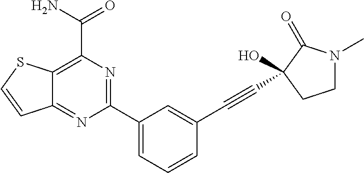 [SMILES: CN1CC[C@](O)(C#Cc2cccc(-c3nc(C(N)=O)c4sccc4n3)c2)C1=O]

### Step 1: Synthesis of 2-chloro-4-(1-ethoxyethenyl)thieno[3,2-d]pyrimidine

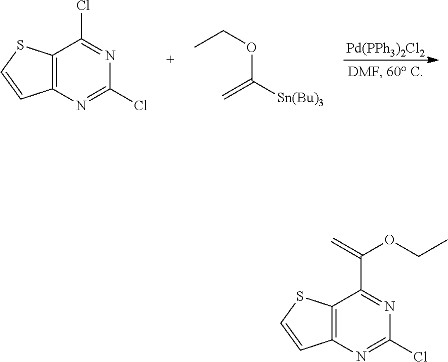

Similar to as described in General Procedure Q, 2,4-dichlorothieno[3,2-d]pyrimidine was reacted with tributyl(1-ethoxyethenyl)stannane to give the title compound (500 mg, 68%) as a white solid. LC-MS (ES, m/z): 241 [M+H]⁺.

### Step 2

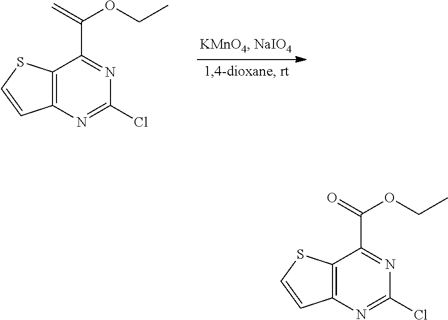

Similar to as described in General Procedure R, 2-chloro-4-(1-ethoxyethenyl)thieno[3,2-d]pyrimidine was reacted with potassium permanganate to give the title compound (200 mg, 40%) as a white solid. LC-MS (ES, m/z): 243 [M+H]⁺.

### Step 3: Synthesis of ethyl 2-(3-bromophenyl)thieno[3,2-d]pyrimidine-4-carboxylate

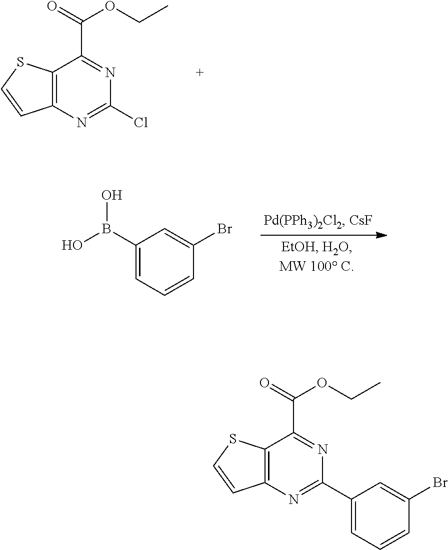 [SMILES: CCOC(=O)c1nc(Cl)nc2ccsc12 ; OB(O)c1cccc(Br)c1 ; CCOC(=O)c1nc(-c2cccc(Br)c2)nc2ccsc12]

Similar to as described in General Procedure M, 2-chlorothieno[3,2-d]pyrimidine-4-carboxylate was reacted with (3-bromophenyl)boronic acid to give the title compound (80 mg, 33%) as a white solid. LC-MS (ES, m/z): 363, 365 [M+H]⁺.

### Step 4: Synthesis of ethyl 2-(3-[2-[(3R)-3-hydroxy-1-methyl-2-oxopyrrolidin-3-yl]ethynyl]phenyl)thieno[3,2-d]pyrimidine-4-carboxylate

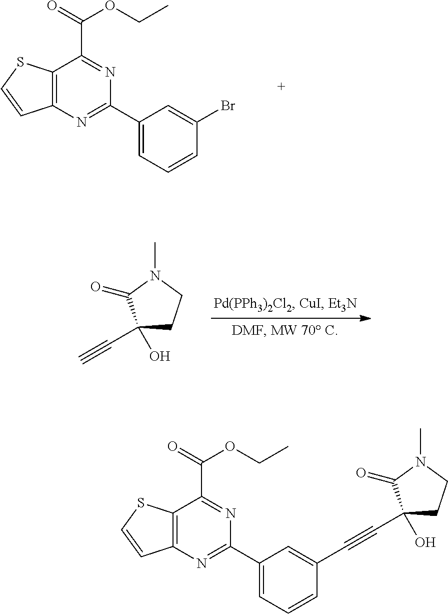

Similar to as described in General Procedure F, ethyl 2-(3-bromophenyl)thieno[3,2-d]pyrimidine-4-carboxylate was reacted with (3-bromophenyl)boronic acid to give the title compound (65 mg, 70%) as yellow oil. LC-MS (ES, m/z): 422 [M+H]⁺.

### Step 5: Synthesis of 2-(3-[2-[(3R)-3-hydroxy-1-methyl-2-oxopyrrolidin-3-yl]ethynyl]phenyl)thieno[3,2-d]pyrimidine-4-carboxamide

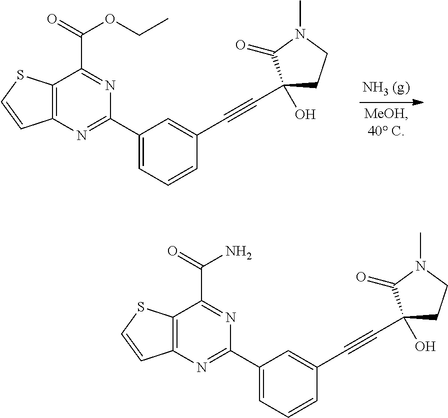 [SMILES: CCOC(=O)c1nc(-c2cccc(C#C[C@]3(O)CCN(C)C3=O)c2)nc2ccsc12 ; CN1CC[C@@](O)(C#Cc2cccc(-c3nc(C(N)=O)c4sccc4n3)c2)C1=O]

Similar to as described in General Procedure S, 2-(3-[2-[(3R)-3-hydroxy-1-methyl-2-oxopyrrolidin-3-yl]ethynyl]phenyl)thieno[3,2-d]pyrimidine-4-carboxylate was reacted with ammonia in methanol to give the title compound (28.7 mg, 47%) as a white solid. LC-MS (ES, m/z): 393 [M+H]⁺. ¹H NMR (400 MHz, CD₃OD) δ 8.79-8.78 (m, 1H), 8.70-8.68 (m, 1H), 8.46 (d, J=5.6 Hz, 1H), 7.68 (d, J=5.6 Hz, 1H), 7.64-7.62 (m, 1H), 7.58-7.54 (m, 1H), 3.57-3.48 (m, 2H), 2.91 (s, 3H), 2.66-2.62 (m, 1H), 2.39-2.33 (m, 1H).

### Example JJ

### Synthesis of 4-(3-[2-[(3R)-3-hydroxy-1-methyl-2-oxopyrrolidin-3-yl]ethynyl]phenyl)thieno[3,2-d]pyrimidine-2-carboxamide

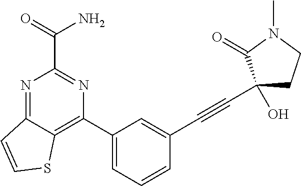 [SMILES: CN1CC[C@@](O)(C#Cc2cccc(-c3nc(C(N)=O)nc4ccsc34)c2)C1=O]

### Step 1: Synthesis of ethyl 4-hydroxythieno[3,2-d]pyrimidine-2-carboxylate

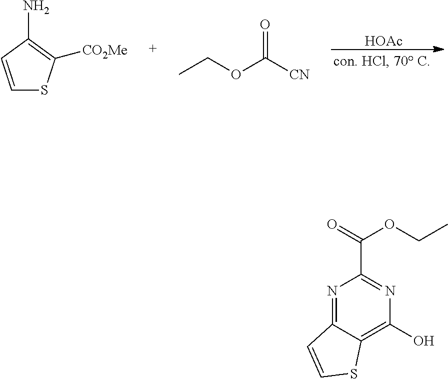

To a solution of methyl 3-aminothiophene-2-carboxylate (6 g, 38.17 mmol, 1.00 equiv) in acetic acid (50 mL) was added 2-ethoxy-2-oxoacetonitrile (7.5 g, 75.69 mmol, 2.00 equiv) and con. hydrochloric acid (5 mL). The mixture was heated at 70° C. for 3 h and then was allowed to cool to room temperature. The solid was collected by filtration and washed with water (50 mL). The pH of the filtrate was adjusted to about 5 by addition of 1N sodium hydroxide solution. The precipitated solid was collected by filtration and washed with water. The solid was dried under high vacuum to afford 5.2 g (61%) of the title compound as a white solid. LC-MS (ES, m/z): 225 [M+H]⁺.

### Step 2: Synthesis of ethyl 4-chlorothieno[3,2-d]pyrimidine-2-carboxylate

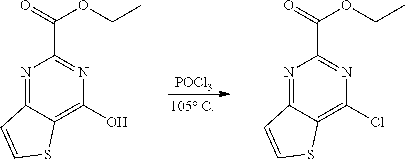

A mixture of ethyl 4-hydroxythieno[3,2-d]pyrimidine-2-carboxylate (3 g, 13.38 mmol, 1.00 equiv) in phosphorus oxychloride (40 mL) was heated at 105° C. for 5 h. After completion of reaction the mixture was concentrated under vacuum. Toluene was added to the concentrated mixture and the solvent was evaporated under vacuum. The residue was purified by silica gel column chromatography eluting with ethyl acetate/petroleum ether (1:2) to afford 2.7 g (83%) of the title compound as a white solid. LC-MS (ES, m/z): 243 [M+H]⁺.

### Step 3: Synthesis of ethyl 4-(3-bromophenyl)thieno[3,2-d]pyrimidine-2-carboxylate

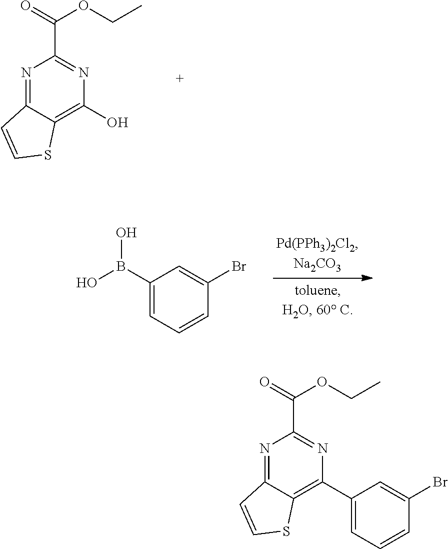 [SMILES: CCOC(=O)c1nc(O)c2sccc2n1 ; OB(O)c1cccc(Br)c1 ; CCOC(=O)c1nc(-c2cccc(Br)c2)c2sccc2n1]

Similar to as described in General Procedure M, ethyl 4-chlorothieno[3,2-d]pyrimidine-2-carboxylate was reacted with (3-bromophenyl)boronic acid to give the title compound (320 mg, 43%) as a white solid. LC-MS (ES, m/z): 363, 365 [M+H]⁺.

### Step 4: Synthesis of ethyl 4-(3-[2-[(3R)-3-hydroxy-1-methyl-2-oxopyrrolidin-3-yl]ethynyl]phenyl)thieno[3,2-d]pyrimidine-2-carboxylate

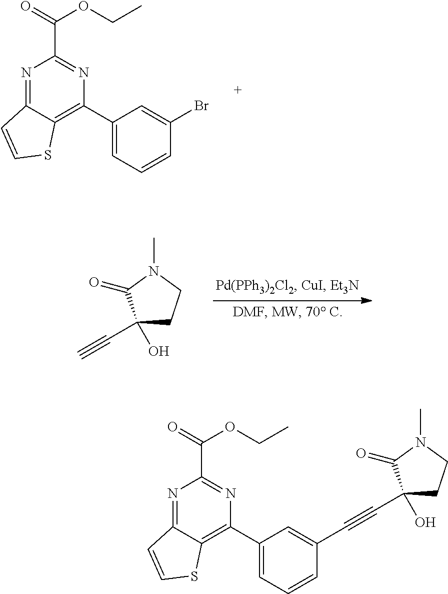

Similar to as described in General Procedure E, ethyl 4-(3-bromophenyl)thieno[3,2-d]pyrimidine-2-carboxylate was reacted with (3R)-3-ethynyl-3-hydroxy-1-methylpyrrolidin-2-one to give the title compound (330 mg, 95%) as a yellow solid. LC-MS (ES, m/z): 422 [M+H]⁺.

### Step 5: Synthesis of 4-(3-[2-[(3R)-3-hydroxy-1-methyl-2-oxopyrrolidin-3-yl]ethynyl]phenyl)thieno[3,2-d]pyrimidine-2-carboxamide

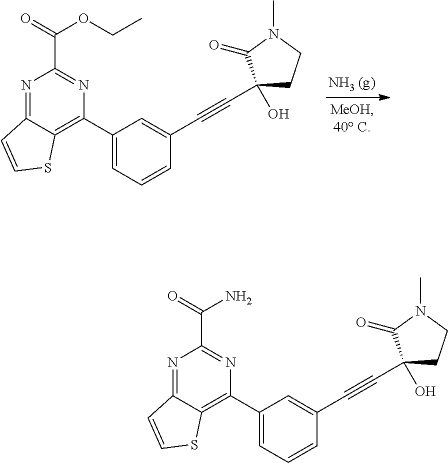 [SMILES: CCOC(=O)c1nc(-c2cccc(C#C[C@]3(O)CCN(C)C3=O)c2)c2sccc2n1 ; CN1CC[C@@](O)(C#Cc2cccc(-c3nc(C(N)=O)nc4ccsc34)c2)C1=O]

Similar to as described in General Procedure S, ethyl 4-(3-[2-[(3R)-3-hydroxy-1-methyl-2-oxopyrrolidin-3-yl]ethynyl]phenyl)thieno[3,2-d]pyrimidine-2-carboxylate was reacted with ammonia in methanol to give the title compound (136.8 mg, 45%) as a white solid. LC-MS (ES, m/z): 393 [M+H]⁺. ¹H NMR (300 MHz, CD₃OD) δ 8.53 (d, J=5.7 Hz, 1H), 8.45 (s, 1H), 8.35-8.33 (m, 1H), 7.83 (d, J=5.7 Hz, 1H), 7.76-7.74 (m, 1H), 7.70-7.68 (m, 1H), 3.51-3.51 (m, 2H), 2.97 (s, 3H), 2.69-2.46 (m, 1H), 2.41-2.30 (m, 1H).

### Example KK

### Synthesis of 2-(3-[2-[(3R)-3-hydroxy-1-methyl-2-oxopyrrolidin-3-yl]ethynyl]phenyl)-5H,6H,7H-cyclopenta[d]pyrimidine-4-carboxamide

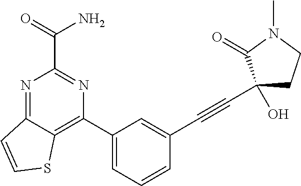 [SMILES: CN1CC[C@@](O)(C#Cc2cccc(-c3nc(C(N)=O)nc4ccsc34)c2)C1=O]

### Step 1: Synthesis of 2-chloro-4-(1-ethoxyethenyl)-5H, 6H,7H-cyclopenta[d]pyrimidine

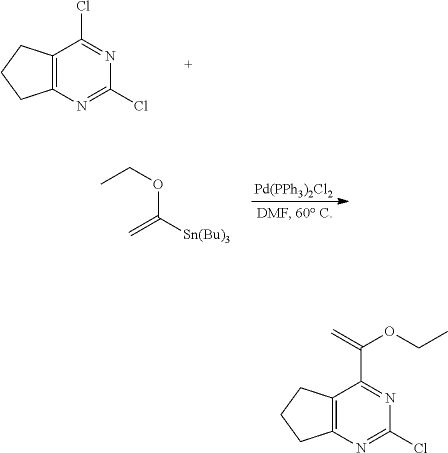

Similar to as described in General Procedure Q, 2,4-dichloro-5H,6H,7H-cyclopenta[d]pyrimidine was reacted with tributyl(1-ethoxyethenyl)stannane to give the title compound (1.7 g, 29%) as a white solid. LC-MS (ES, m/z): 225 [M+H]⁺.

### Step 2: Synthesis of ethyl 2-chloro-5H,6H,7H-cyclopenta[d]pyrimidine-4-carboxylate

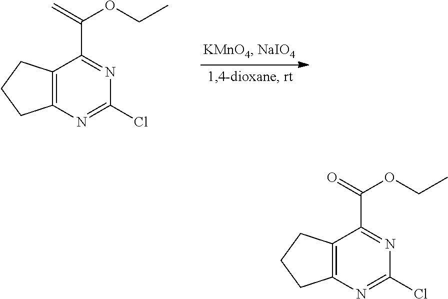

Similar to as described in General Procedure R, 2-chloro-4-(1-ethoxyethenyl)-5H,6H,7H-cyclopenta[d]pyrimidine was reacted with potassium permanganate to give the title compound (1.1 g, 64%) as a white solid. LC-MS (ES, m/z): 227 [M+H]⁺.

### Step 3: Synthesis of ethyl 2-(3-bromophenyl)-5H,6H,7H-cyclopenta[d]pyrimidine-4-carboxylate

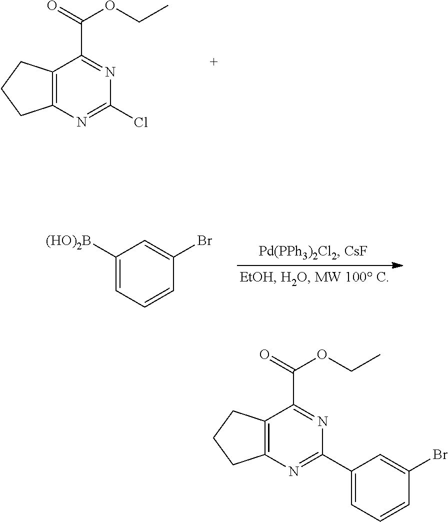 [SMILES: CCOC(=O)c1nc(Cl)nc2c1CCC2 ; OB(O)c1cccc(Br)c1 ; CCOC(=O)c1nc(-c2cccc(Br)c2)nc2c1CCC2]

Similar to as described in General Procedure M, ethyl 2-chloro-5H,6H,7H-cyclopenta[d]pyrimidine-4-carboxylate was reacted with ethyl 2-chloro-5H,6H,7H-cyclopenta[d]pyrimidine-4-carboxylate to give the title compound (300 mg, 18%) as a white solid. LC-MS (ES, m/z): 347, 349 [M+H]⁺.

### Step 4: Synthesis of ethyl 2-(3-[2-[(3R)-3-hydroxy-1-methyl-2-oxopyrrolidin-3-yl]ethynyl]phenyl)-5H,6H,7H-cyclopenta[d]pyrimidine-4-carboxylate

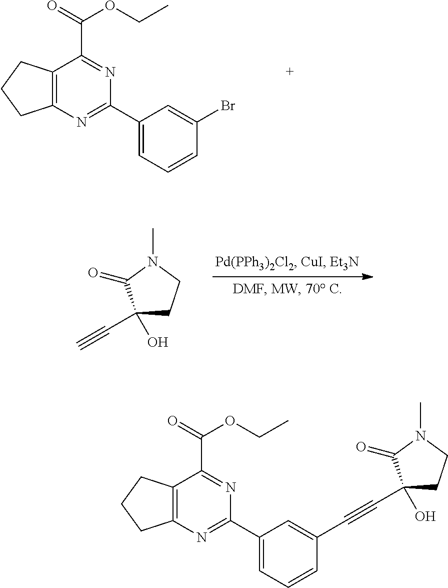

Similar to as described in General Procedure F, ethyl 2-(3-bromophenyl)-5H,6H,7H-cyclopenta[d]pyrimidine-4-carboxylate was reacted with (3R)-3-ethynyl-3-hydroxy-1-methylpyrrolidin-2-one to give the title compound (330 mg, 94%) as a yellow solid. LC-MS (ES, m/z): 406 [M+H]⁺.

### Step 5: Synthesis of 2-(3-[2-[(3R)-3-hydroxy-1-methyl-2-oxopyrrolidin-3-yl]ethynyl]phenyl)-5H,6H,7H-cyclopenta[d]pyrimidine-4-carboxamide

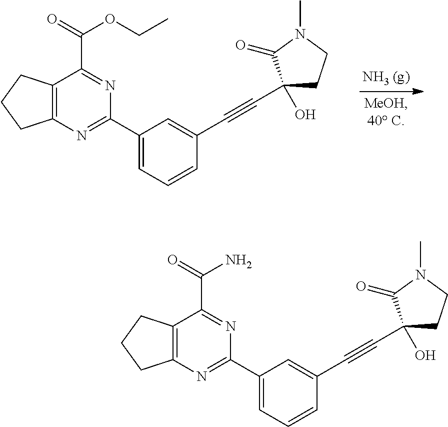 [SMILES: CCOC(=O)c1nc(-c2cccc(C#C[C@]3(O)CCN(C)C3=O)c2)nc2c1CCC2 ; CN1CC[C@@](O)(C#Cc2cccc(-c3nc4c(c(C(N)=O)n3)CCC4)c2)C1=O]

Similar to as described in General Procedure S, ethyl 2-(3-[2-[(3R)-3-hydroxy-1-methyl-2-oxopyrrolidin-3-yl]ethynyl]phenyl)-5H,6H,7H-cyclopenta[d]pyrimidine-4-carboxylate was reacted with ammonia in methanol to give the title compound as a white solid. LC-MS (ES, m/z): 377 [M+H]⁺. ¹H NMR (400 MHz, CD₃OD) δ 8.63 (s, 1H), 8.53 (d, J=8.0 Hz, 1H), 7.59 (d, J=8.0 Hz, 1H), 7.51 (t, J=8.0 Hz, 1H), 3.56-3.47 (m, 2H), 3.41 (t, J=7.6 Hz, 2H), 3.11 (t, J=7.6 Hz, 2H), 2.96 (s, 3H), 2.66-2.62 (m, 1H), 2.38-2.33 (m, 1H), 2.28-2.20 (m, 2H).

### Example LL

### Synthesis of 7-fluoro-2-(3-[2-[(3R)-3-hydroxy-1-methyl-2-oxopyrrolidin-3-yl]ethynyl]phenyl)quinazoline-4-carboxamide

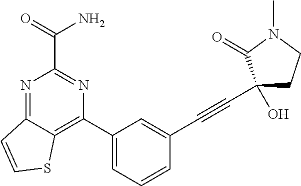 [SMILES: CN1CC[C@@](O)(C#Cc2cccc(-c3nc(C(N)=O)nc4ccsc34)c2)C1=O]

### Step 1: Synthesis of 2-chloro-4-(1-ethoxyethenyl)-7-fluoroquinazoline

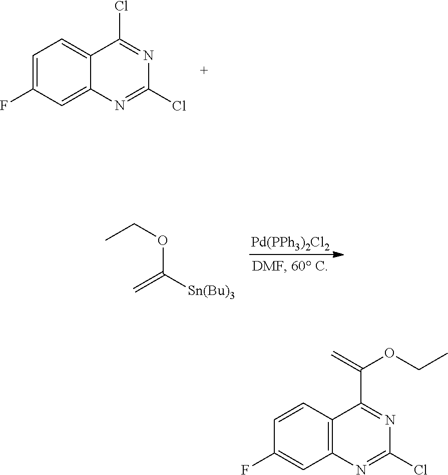

Similar to as described in General Procedure Q, 2,4-dichloro-7-fluoroquinazoline was reacted with tributyl(1-ethoxyethenyl)stannane to give the title compound (378 mg, 32%) as a light yellow solid. LC-MS (ES, m/z): 253 [M+H]⁺.

### Step 2: Synthesis of ethyl 2-chloro-7-fluoroquinazoline-4-carboxylate

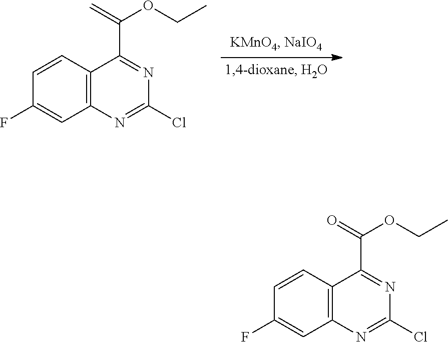 [SMILES: C=C(OCC)c1nc(Cl)nc2cc(F)ccc12 ; CCOC(=O)c1nc(Cl)nc2cc(F)ccc12]

Similar to as described in General Procedure R, 2-chloro-4-(1-ethoxyethenyl)-7-fluoroquinazoline was reacted with potassium permanganate to give the title compound (254 mg, 67%) as an off-white solid. LC-MS (ES, m/z): 255 [M+H]⁺.

### Step 3: Synthesis of ethyl 2-(3-bromophenyl)-7-fluoroquinazoline-4-carboxylate

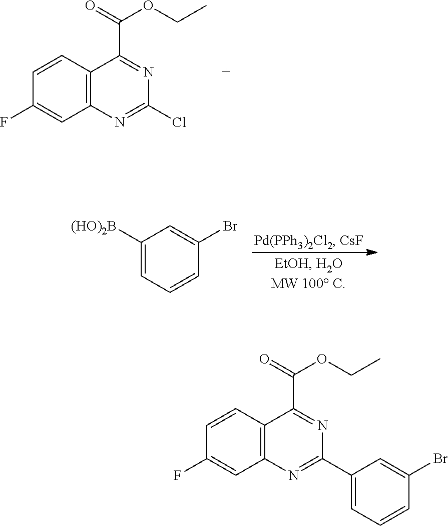

Similar to as described in General Procedure M, 2-chloro-4-(1-ethoxyethenyl)-7-fluoroquinazoline was reacted with (3-bromophenyl)boronic acid to give the title compound (196 mg, 61%) as an off-white solid (crude, 91% purity). LC-MS (ES, m/z): 375, 377 [M+H]⁺.

### Step 4: Synthesis of ethyl 7-fluoro-2-(3-[2-[(3R)-3-hydroxy-1-methyl-2-oxopyrrolidin-3-yl]ethynyl]phenyl)quinazolin phenyl)quinazoline-4-carboxylate

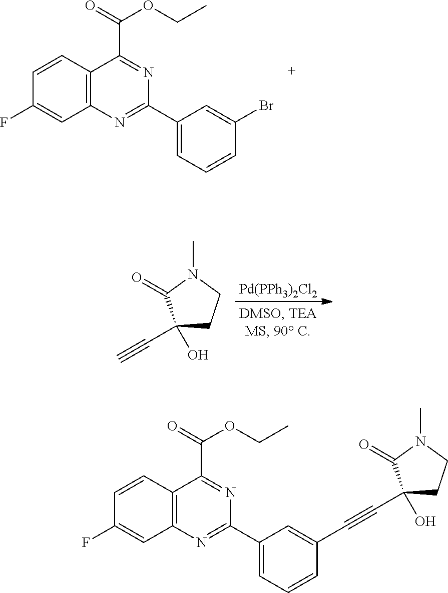

Similar to as described in General Procedure M, ethyl 2-(3-bromophenyl)-7-fluoroquinazoline-4-carboxylate was reacted with (3R)-3-ethynyl-3-hydroxy-1-methylpyrrolidin-2-one to give the title compound (100 mg, 87%) as a light yellow solid (crude, 72% purity). LC-MS (ES, m/z): 434 [M+H]⁺.

### Step 5: Synthesis of 7-fluoro-2-(3-[2-[(3R)-3-hydroxy-1-methyl-2-oxopyrrolidin-3-yl]ethynyl]phenyl)quinazolin phenyl)quinazoline-4-carboxamide

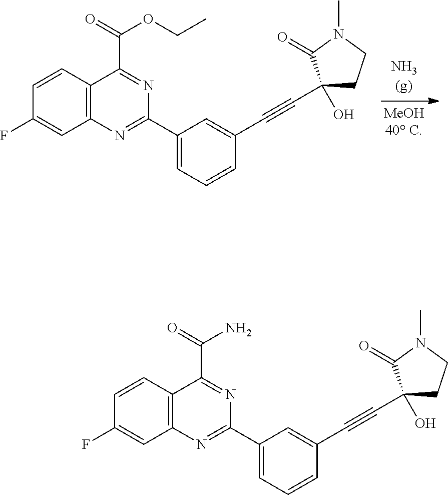 [SMILES: CCOC(=O)c1nc(-c2cccc(C#C[C@]3(O)CCN(C)C3=O)c2)nc2cc(F)ccc12 ; CN1CC[C@@](O)(C#Cc2cccc(-c3nc(C(N)=O)c4ccc(F)cc4n3)c2)C1=O]

Similar to as described in General Procedure S, ethyl 7-fluoro-2-(3-[2-[(3R)-3-hydroxy-1-methyl-2-oxopyrrolidin-3-yl]ethynyl]phenyl)quinazolin phenyl)quinazoline-4-carboxylate was reacted with ammonia in methanol to give the title compound (31.6 mg, 34%) as an off-white solid. LC-MS-(ES, m/z): 405 [M+H]⁺. ¹H NMR (300 MHz, CD₃OD) δ 9.00 (m, 1H), 8.65 (t, J=1.5 Hz, 1H), 8.58-8.54 (m, 1H), 7.67 (dd, J=9.9, 2.4 Hz, 1H), 7.55-7.42 (m, 3H), 3.44-3.38 (m, 2H), 2.86 (s, 3H), 2.56-2.50 (m, 1H), 2.29-2.20 (m, 1H).

### Example MM

### Synthesis of 2-(3-[2-[(3R)-3-hydroxy-1-methyl-2-oxopyrrolidin-3-yl]ethynyl]phenyl)-7-methoxyquinazoline-4-carboxamide

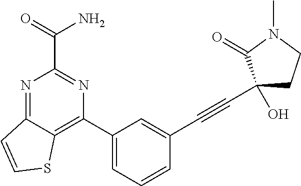 [SMILES: CN1CC[C@@](O)(C#Cc2cccc(-c3nc(C(N)=O)nc4ccsc34)c2)C1=O]

### Step 1: Synthesis of 2-chloro-4-(1-ethoxyethenyl)-7-methoxyquinazoline

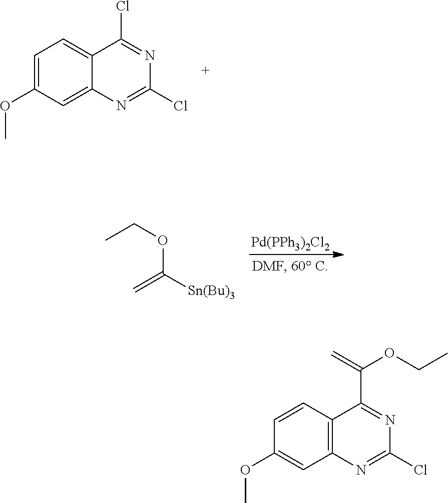

Similar to as described in General Procedure Q, 2,4-dichloro-7-methoxyquinazoline was reacted with tributyl(1-ethoxyethenyl)stannane to give the title compound (650 mg, 61%) as an off-white solid. LC-MS (ES, m/z): 265 [M+H]⁺.

### Step 2: Synthesis of methyl 2-chloro-7-methoxyquinazoline-4-carboxylate

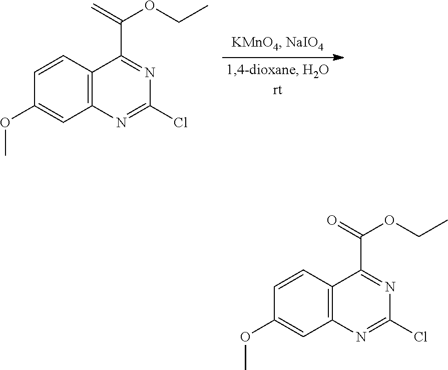 [SMILES: C=C(OCC)c1nc(Cl)nc2cc(OC)ccc12 ; CCOC(=O)c1nc(Cl)nc2cc(OC)ccc12]

Similar to as described in General Procedure R, 2-chloro-7-methoxy-4-(1-methoxyethenyl)quinazoline was reacted with potassium permanganate to give the title compound (362 mg, 76%) as an white solid. LC-MS (ES, m/z): 267 [M+H]⁺.

### Step 3: Synthesis of ethyl 2-(3-bromophenyl)-7-methoxyquinazoline-4-carboxylate

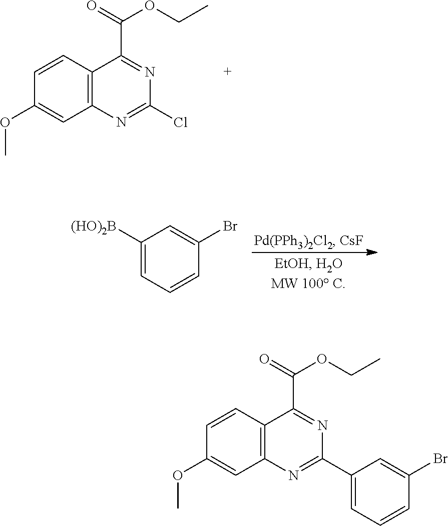

Similar to as described in General Procedure M, 2-chloro-7-methoxyquinazoline-4-carboxylate was reacted with (3-bromophenyl)boronic acid to give the title compound (124 mg, 43%) as an off-white solid (crude, 84% purity). LC-MS (ES, m/z): 387, 389 [M+H]⁺.

### Step 4: Synthesis of ethyl 2-(3-[2-[(3R)-3-hydroxy-1-methyl-2-oxopyrrolidin-3-yl]ethynyl]phenyl)-7-methoxyquinazoline-4-carboxylate

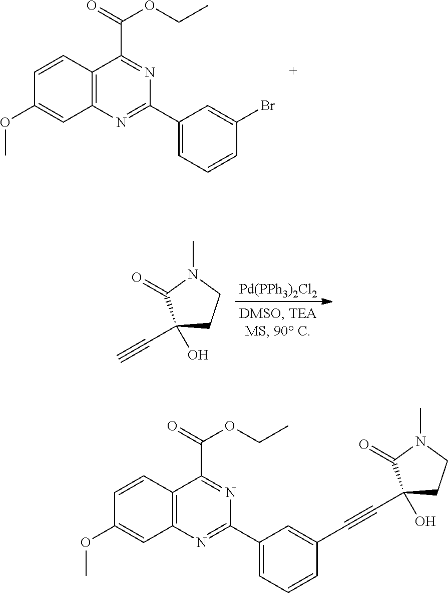

Similar to as described in General Procedure M, ethyl 2-(3-bromophenyl)-7-methoxyquinazoline-4-carboxylate was reacted with (3R)-3-ethynyl-3-hydroxy-1-methylpyrrolidin-2-one to give the title compound (100 mg, 87%) as a light yellow solid (crude, 71% purity). LC-MS (ES, m/z): 446 [M+H]⁺.

### Step 5: Synthesis of 2-(3-[2-[(3R)-3-hydroxy-1-methyl-2-oxopyrrolidin-3-yl]ethynyl]phenyl)-7-methoxyquinazoline-4-carboxamide

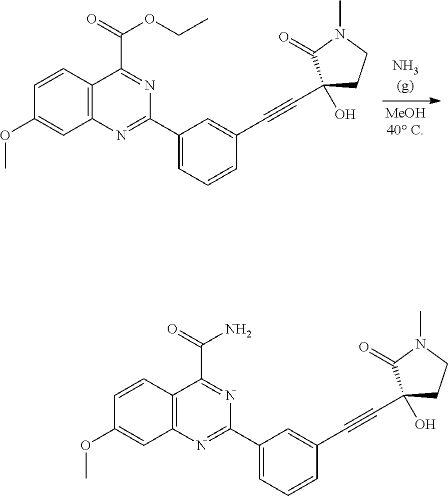 [SMILES: CCOC(=O)c1nc(-c2cccc(C#C[C@]3(O)CCN(C)C3=O)c2)nc2cc(OC)ccc12 ; COc1ccc2c(C(N)=O)nc(-c3cccc(C#C[C@]4(O)CCN(C)C4=O)c3)nc2c1]

Similar to as described in General Procedure S, ethyl 2-(3-[2-[(3R)-3-hydroxy-1-methyl-2-oxopyrrolidin-3-yl]ethynyl]phenyl)-7-methoxyquinazoline-4-carboxylate was reacted with ammonia in methanol to give the title compound (35.2 mg, 37%) as an off-white solid. LC-MS (ES, m/z): 417 [M+H]⁺. ¹H NMR (300 MHz, CD₃OD) δ 8.91 (d, J=9.3 Hz, 1H), 8.76-8.75 (m, 1H), 8.66 (d, J=7.8 Hz, 1H), 7.64-7.61 (m, 1H), 7.57-7.49 (m, 2H), 7.35 (dd, J=9.3, 2.7 Hz, 1H), 4.06 (s, 3H), 3.55-3.49 (m, 2H), 2.96 (s, 3H), 2.68-2.60 (m, 1H), 2.40-2.30 (m, 1H).

### Example NN

### Synthesis of 8-(3-[2-[(3S)-3-hydroxy-1-methyl-2-oxopyrrolidin-3-yl]ethynyl]phenyl)imidazo[1,2-a]pyrazine-6-carboxamide

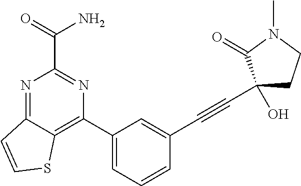 [SMILES: CN1CC[C@@](O)(C#Cc2cccc(-c3nc(C(N)=O)nc4ccsc34)c2)C1=O]

### Step 1: Synthesis of methyl 5-amino-6-bromopyrazine-2-carboxylate

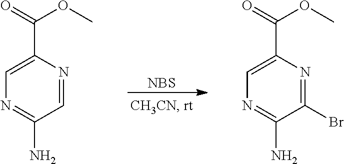

A solution of methyl 5-aminopyrazine-2-carboxylate (2 g, 13.06 mmol, 1.00 equiv), N-bromosuccinimide (2.8 g, 15.73 mmol, 1.20 equiv) in acetonitrile (30 mL) was stirred overnight at room temperature. The reaction mixture was directly concentrated under vacuum and the residue was purified by silica gel chromatography eluting with ethyl acetate/petroleum ether (1:1) to afford 1.6 g (53%) of the title compound as a yellow solid. LC-MS (ES, m/z): 232[M+H]⁺.

### Step 2: Synthesis of methyl 8-bromoimidazo[1,2-a]pyrazine-6-carboxylate

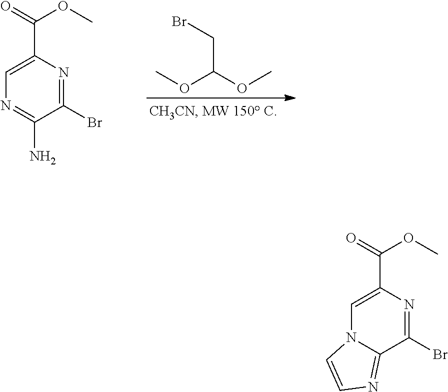

In a sealed tube a solution of methyl 5-amino-6-bromopyrazine-2-carboxylate (200 mg, 0.86 mmol, 1.00 equiv), 2-bromo-1,1-dimethoxyethane (148 mg, 0.88 mmol, 1.00 equiv) in acetonitrile (8 mL) was heated with microwave radiation at 150° C. for 2 h. The reaction mixture was directly concentrated under vacuum and the residue was purified by silica gel chromatography eluting with dichloromethane/methanol (10:1) to afford 20 mg (9%) of the title compound as a yellow solid. LC-MS (ES, m/z): 256[M+H]⁺.

### Step 3: Synthesis of (R)-methyl 8-(3-((3-hydroxy-1-methyl-2-oxopyrrolidin-3-yl)ethynyl)phenyl)imidazo[1,2-a]pyrazine-6-carboxylate

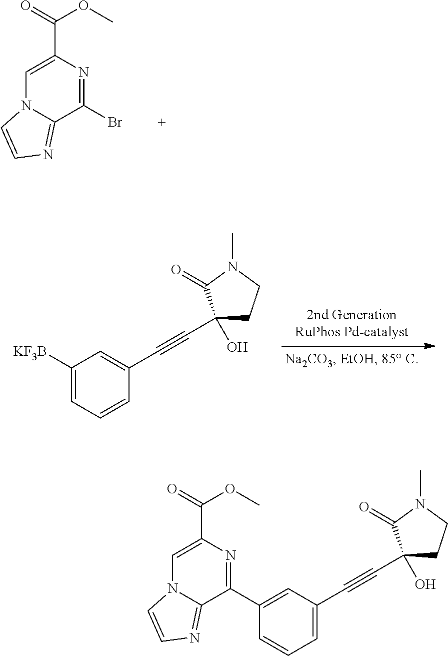

Similar to as described in General Procedure U, methyl 8-bromoimidazo[1,2-a]pyrazine-6-carboxylate was reacted with potassium trifluoro(3-{2-[(3R)-3-hydroxy-1-methyl-2-oxopyrrolidin-3-yl]ethynyl}phenyl)boranuide to give the title compound (60 mg, 26%) as a yellow solid. LC-MS (ES, m/z): 391 [M+H]⁺.

### Step 4: Synthesis of 8-(3-[2-[(3S)-3-hydroxy-1-methyl-2-oxopyrrolidin-3-yl]ethynyl]phenyl)imidazo[1,2-a]pyrazine-6-carboxamide

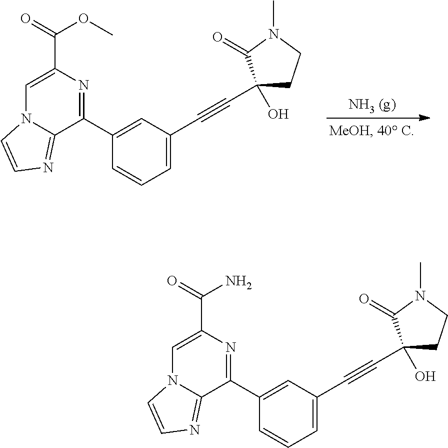 [SMILES: COC(=O)c1cn2ccnc2c(-c2cccc(C#C[C@]3(O)CCN(C)C3=O)c2)n1 ; CN1CC[C@@](O)(C#Cc2cccc(-c3nc(C(N)=O)cn4ccnc34)c2)C1=O]

Similar to as described in General Procedure S, methyl 1-(3-[2-[(3R)-3-hydroxy-1-methyl-2-oxopyrrolidin-3-yl]ethynyl]phenyl)-4-(methylamino)-1H-pyrazolo[4,3-c]pyridine-3-carboxylate was reacted with ammonia in methanol to give the title compound (10.5 mg, 19%) as a white solid. LC-MS (ES, m/z): 375 [M+H]⁺. ¹H NMR (400 MHz, CD₃OD) δ 9.20 (s, 1H), 8.88 (d, J=1.2 Hz, 1H), 8.77 (dd, J=6.4, 1.6 Hz, 1H), 8.25 (d, J=1.2 Hz, 1H), 7.94 (d, J=1.2 Hz, 1H), 7.67 (dd, J=6.4, 1.6 Hz, 1H), 7.59 (t, J=4.0 Hz, 1H), 3.56-3.47 (m, 3H), 2.96 (s, 3H), 2.67-2.61 (m, 1H), 2.39-2.32 (m, 1H).

### Example OO

### Synthesis of 1-(3-[2-[(3R)-3-hydroxy-1-methyl-2-oxopyrrolidin-3-yl]ethynyl]phenyl)-5H,6H,7H-cyclopenta[c]pyridine-3-carboxamide

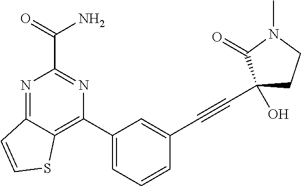 [SMILES: CN1CC[C@@](O)(C#Cc2cccc(-c3nc(C(N)=O)nc4ccsc34)c2)C1=O]

### Step 1: Synthesis of 3-(ethoxycarbonyl)-5H,6H,7H-cyclopenta[c]pyridin-2-ium-2-olate

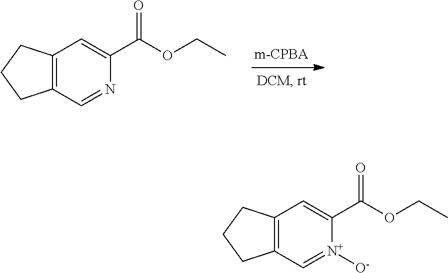

A solution of ethyl 5H,6H,7H-cyclopenta[c]pyridine-3-carboxylate (200 mg, 1.05 mmol, 1.00 equiv), m-CPBA (304 mg, 1.76 mmol, 1.70 equiv) in dichloromethane (5 mL) was stirred overnight at room temperature. The reaction was then quenched with saturated sodium sulfite solution, extracted with dichloromethane. The combined organic layers were dried over anhydrous sodium sulfate and concentrated under vacuum to give 220 mg (crude) of the title compound as a light yellow solid. LC-MS (ES, m/z): 208[M+H]⁺.

### Step 2: Synthesis of ethyl 1-chloro-5H,6H,7H-cyclopenta[c]pyridine-3-carboxylate

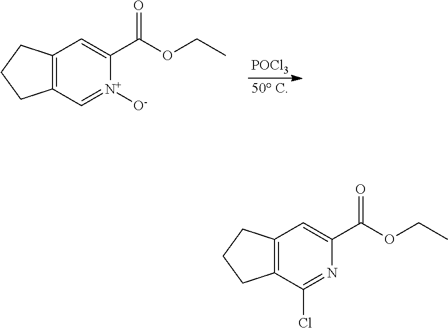

A solution of 3-(ethoxycarbonyl)-5H,6H,7H-cyclopenta[c]pyridin-2-ium-2-olate (220 mg, 1.06 mmol, 1.00 equiv) in phosphoroxychloride (3 mL) was stirred overnight at 50° C. and quenched with water (1 mL). The mixture was adjusted to pH 8 with saturated sodium carbonate. The mixture was extracted with ethyl acetate, dried over anhydrous sodium sulfate, and concentrated under vacuum. The residue was purified by silica gel chromatography eluting with ethyl acetate/petroleum ether (1:20) to afford 190 mg (79%) of the title compound as a yellow solid. LC-MS (ES, m/z): 226[M+H]⁺.

### Step 3: Synthesis of ethyl 1-(3-[2-[(3R)-3-hydroxy-1-methyl-2-oxopyrrolidin-3-yl]ethynyl]phenyl)-5H,6H,7H-cyclopenta[c]pyridine-3-carboxylate

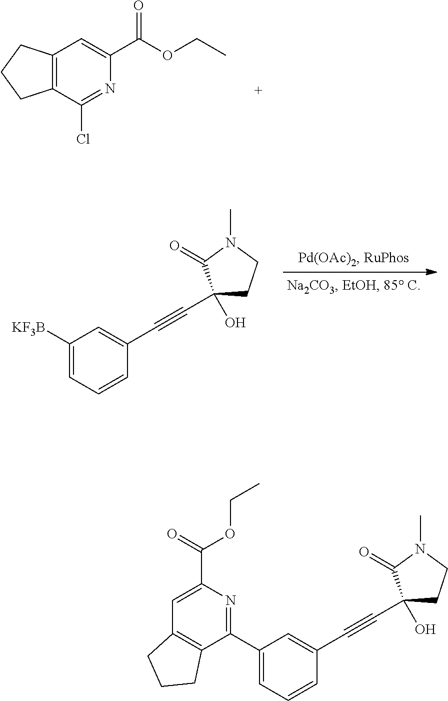

Similar to as described in General Procedure U, ethyl 1-chloro-5H,6H,7H-cyclopenta[c]pyridine-3-carboxylate was reacted with potassium trifluoro(3-{2-[(3R)-3-hydroxy-1-methyl-2-oxopyrrolidin-3-yl]ethynyl}phenyl)boranuide to give the title compound (32 mg, 10%) as a yellow oil. LC-MS (ES, m/z): 405[M+H]⁺.

### Step 4: Synthesis of 1-(3-[2-[(3R)-3-hydroxy-1-methyl-2-oxopyrrolidin-3-yl]ethynyl]phenyl)-5H,6H, 7H-cyclopenta[c]pyridine-3-carboxamide

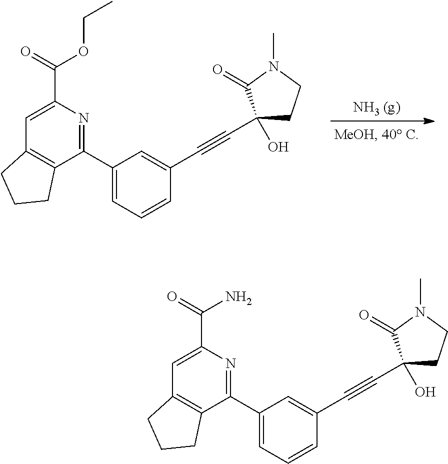 [SMILES: CCOC(=O)c1cc2c(c(-c3cccc(C#C[C@]4(O)CCN(C)C4=O)c3)n1)CCC2 ; CN1CC[C@@](O)(C#Cc2cccc(-c3nc(C(N)=O)cc4c3CCC4)c2)C1=O]

Similar to as described in General Procedure S, ethyl 1-(3-[2-[(3R)-3-hydroxy-1-methyl-2-oxopyrrolidin-3-yl]ethynyl]phenyl)-5H,6H,7H-cyclopenta[c]pyridine-3-carboxylate was reacted with ammonia in methanol to give the title compound as a white solid. LC-MS (ES, m/z): 376[M+H]⁺. ¹H NMR (400 MHz, CDCl₃) δ 8.10-8.06 (m, 2H), 7.84 (s, 1H), 7.73-7.70 (m, 1H), 7.60-7.39 (m, 1H), 5.62 (s, 1H), 3.55-3.47 (m, 1H), 3.42-3.35 (m, 1H), 3.14-3.02 (m, 4H), 2.97 (s, 1H), 2.70-2.62 (m, 1H), 2.43-2.33 (m, 1H), 2.20-2.11 (m, 2H).

### Example PP

### Synthesis of 8-fluoro-2-(3-[2-[(3R)-3-hydroxy-1-methyl-2-oxopyrrolidin-3-yl]ethynyl]phenyl)quinazoline-4-carboxamide

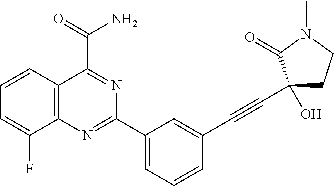 [SMILES: CN1CC[C@@](O)(C#Cc2cccc(-c3nc(C(N)=O)c4cccc(F)c4n3)c2)C1=O]

### Step 1: Synthesis of 2-chloro-4-(1-ethoxyethenyl)-8-fluoroquinazoline

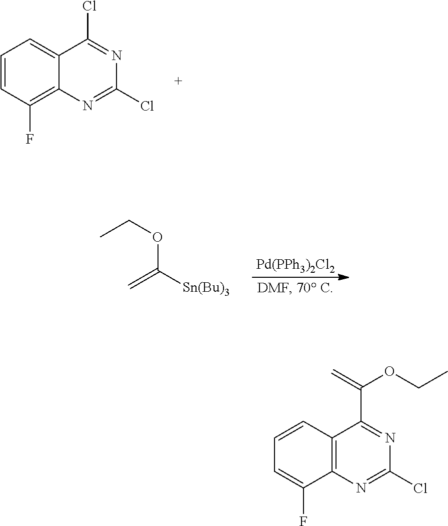

Similar to as described in General Procedure Q, 2,4-dichloro-8-fluoroquinazoline was reacted with tributyl(1-ethoxyethenyl)stannane to give the title compound (600 mg) as an off-white solid. LC-MS (ES, m/z): 253 [M+H]⁺.

### Step 2: Synthesis of ethyl 2-chloro-8-fluoroquinazoline-4-carboxylate

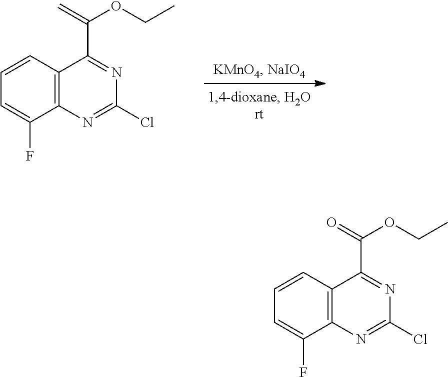 [SMILES: C=C(OCC)c1nc(Cl)nc2c(F)cccc12 ; CCOC(=O)c1nc(Cl)nc2c(F)cccc12]

Similar to as described in General Procedure R, 2-chloro-4-(1-ethoxyethenyl)-8-fluoroquinazoline was reacted with potassium permanganate to give the title compound (140 mg) as an off-white solid. LC-MS (ES, m/z): 255 [M+H]⁺.

### Step 3: Synthesis of ethyl 2-(3-bromophenyl)-8-fluoroquinazoline-4-carboxylate

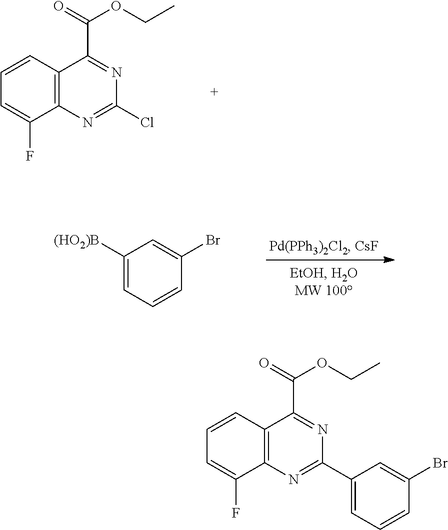

Similar to as described in General Procedure M, ethyl 2-chloro-8-fluoroquinazoline-4-carboxylate was reacted with (3-bromophenyl)boronic acid to give the title compound (80 mg) as an off-white solid (crude, 71% purity). LC-MS (ES, m/z): 375, 377 [M+H]⁺.

### Step 4: Synthesis of ethyl 8-fluoro-2-(3-[2-[(3R)-3-hydroxy-1-methyl-2-oxopyrrolidin-3-yl]ethynyl]phenyl)quinazoline-4-carboxylate

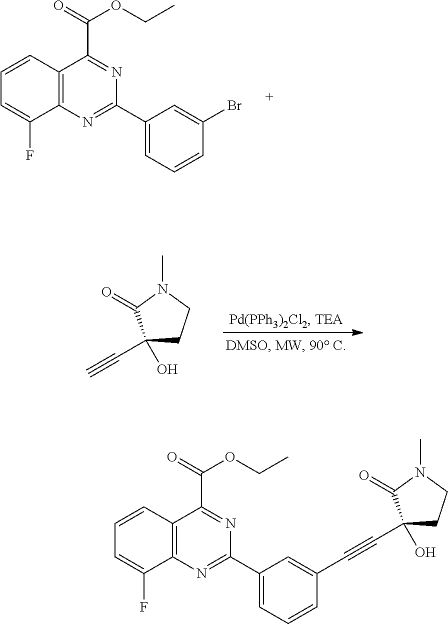

Similar to as described in General Procedure E, ethyl 2-(3-bromophenyl)-8-fluoroquinazoline-4-carboxylate was reacted with (3R)-3-ethynyl-3-hydroxy-1-methylpyrrolidin-2-one to give the title compound (70 mg) as an off-white solid (crude, 64% purity). LC-MS (ES, m/z): 434 [M+H]⁺.

### Step 5: Synthesis of 8-fluoro-2-(3-[2-[(3R)-3-hydroxy-1-methyl-2-oxopyrrolidin-3-yl]ethynyl]phenyl)quinazoline-4-carboxamide

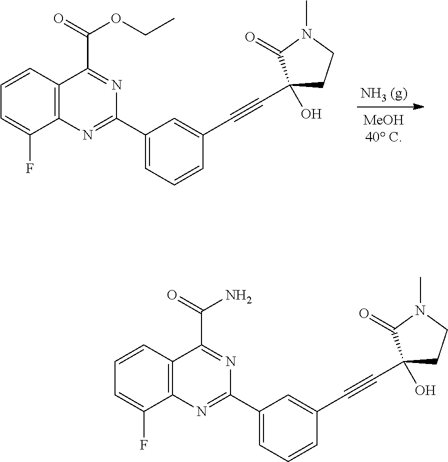 [SMILES: CCOC(=O)c1nc(-c2cccc(C#C[C@]3(O)CCN(C)C3=O)c2)nc2c(F)cccc12 ; CN1CC[C@@](O)(C#Cc2cccc(-c3nc(C(N)=O)c4cccc(F)c4n3)c2)C1=O]

Similar to as described in General Procedure S, ethyl 8-fluoro-2-(3-[2-[(3R)-3-hydroxy-1-methyl-2-oxopyrrolidin-3-yl]ethynyl]phenyl)quinazoline-4-carboxylate was reacted with ammonia in methanol to give the title compound (9.1 mg) as a white solid. LC-MS (ES, m/z): 405 [M+H]⁺. ¹H NMR (300 MHz, CD₃OD) δ 8.80-8.70 (m, 3H), 7.81-7.64 (m, 3H), 7.60-7.57 (m, 1H), 3.52-3.50 (m, 2H), 2.96 (s, 3H), 2.68-2.61 (m, 1H), 2.40-2.30 (m, 1H).

### Example QQ

### Synthesis of 2-(3-[2-[(3R)-3-hydroxy-1-methyl-2-oxopyrrolidin-3-yl]ethynyl]phenyl)-6,6-dim ethyl-5,6,7,8-tetrahydroquinazoline-4-carboxamide

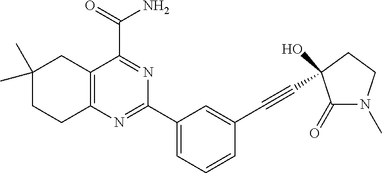

### Step 1: Synthesis of ethyl 2-(5,5-dimethyl-2-oxocyclohexyl)-2-oxoacetate

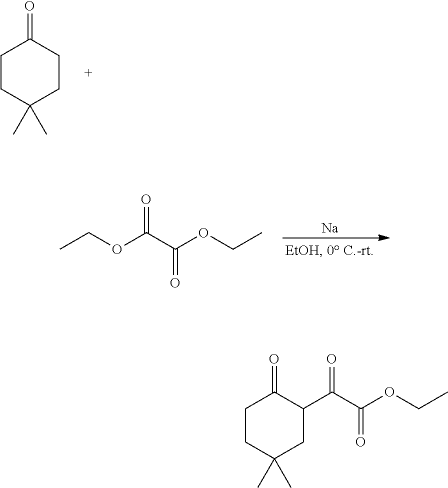

Similar to as described in General Procedure Y Step 1, 4,4-dimethylcyclohexan-1-one was reacted with diethyl oxalate to give the title compound (3.5 g, 20%) of as a white solid which was used for the next step without further purification. LC-MS (ES, m/z): 227 [M+H]⁺.

### Step 2: Synthesis of ethyl 2-(3-bromophenyl)-6,6-dimethyl-5,6,7,8-tetrahydroquinazoline-4-carboxylate

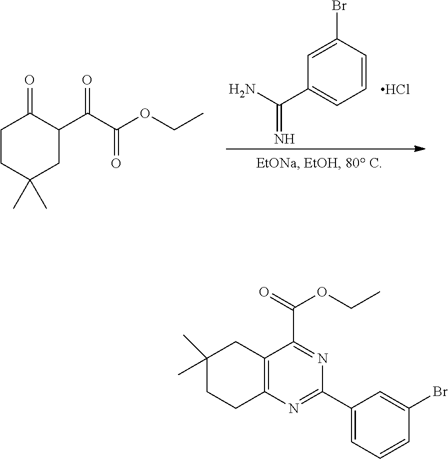 [SMILES: CCOC(=O)C(=O)C1CC(C)(C)CCC1=O ; N=C(N)c1cccc(Br)c1 ; Cl ; CCOC(=O)c1nc(-c2cccc(Br)c2)nc2c1CC(C)(C)CC2]

A solution of 3-bromobenzene-1-carboximidamide hydrochloride (876.1 mg, 3.72 mmol, 0.60 equiv), ethyl 2-(5,5-dimethyl-2-oxocyclohexyl)-2-oxoacetate (1.5 g, 6.63 mmol, 1.00 equiv) and sodium ethoxide (272 mg, 4.00 mmol, 1.1 equiv) in ethanol (20 mL) was stirred for 15 min at 0° C. and then 80° C. for 2 h. The reaction was quenched by saturated ammonium chloride and the pH value of the solution was adjusted to 6 with 1N hydrochloric acid. The mixture was extracted with ethyl acetate, washed with brine, dried over anhydrous sodium sulfate, and concentrated under vacuum. The residue was purified on a silica gel column with ethyl acetate/petroleum ether (1:20) to give the title compound (110 mg, 4%) as a yellow solid. LC-MS: (ES, m/z): 389,391 [M+H]⁺.

### Step 3: Synthesis of ethyl 2-(3-[2-[(3R)-3-hydroxy-1-methyl-2-oxopyrrolidin-3-yl]ethynyl]phenyl)-6,6-dimethyl-5,6,7,8-tetrahydroquinazoline-4-carboxylate

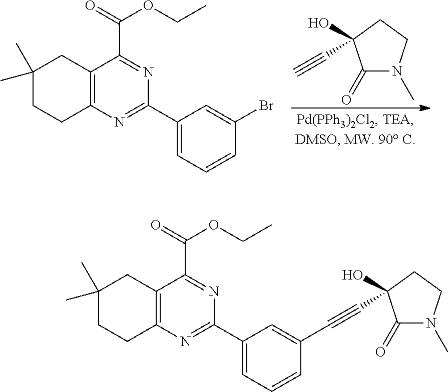

Similar to as described in General Procedure E, ethyl 2-(3-bromophenyl)-6, 6-dim ethyl-5,6,7,8-tetrahydroquinazoline-4-carboxylate was reacted with (3R)-3-ethynyl-3-hydroxy-1-methylpyrrolidin-2-one to give the title compound (100 mg 87%) as a yellow oil. LC-MS (ES, m/z): 448 [M+H]⁺, 489 [M+CH₃CN+H]⁺.

### Step 4: Synthesis of 2-(3-[2-[(3R)-3-hydroxy-1-methyl-2-oxopyrrolidin-3-yl]ethynyl]phenyl)-6,6-dimethyl-5,6,7,8-tetrahydroquinazoline-4-carboxamide

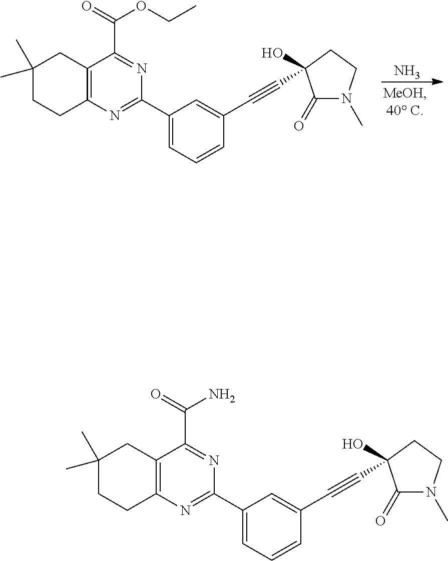

Similar to as described in General Procedure S, ethyl 2-(3-[2-[(3R)-3-hydroxy-1-methyl-2-oxopyrrolidin-3-yl]ethynyl]phenyl)-6,6-dimethyl-5,6,7,8-tetrahydroquinazoline-4-carboxylate was reacted with ammonia in methanol to give the title compound (17.4 mg, 19%) as a white solid. LC-MS (ES, m/z): 419 [M+H]⁺. ¹H NMR (400 MHz, CD₃OD) δ 8.57 (s, 1H), 8.48 (d, J=8.0 Hz, 1H), 7.60-7.45 (m, 2H), 3.55-3.42 (m, 2H), 3.08 (t, J=6.8 Hz, 2H), 2.96 (s, 3H), 2.95 (s, 2H), 2.66-2.60 (m, 1H), 2.38-2.31 (m, 1H), 1.77 (t, J=6.8 Hz, 2H), 1.05 (s, 6H).

### Example RR

### Synthesis of 2-(3-[2-[(3R)-3-hydroxy-1-methyl-2-oxopyrrolidin-3-yl]ethynyl]phenyl)-5,6,7,8-tetrahydroquinazoline-4-carboxamide

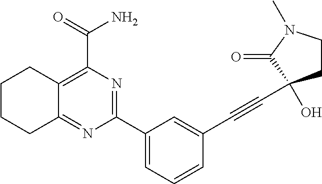 [SMILES: CN1CC[C@@](O)(C#Cc2cccc(-c3nc4c(c(C(N)=O)n3)CCCC4)c2)C1=O]

### Step 1: Synthesis of ethyl 2-oxo-2-(2-oxocyclohexyl)acetate

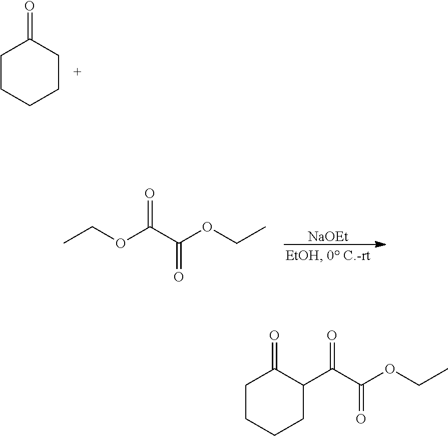

Similar to as described in General Procedure Y Step 1, cyclohexanone was reacted with diethyl oxalate to give the title compound (20 g, crude) as a yellow solid which was used for the next step without further purification. LC-MS (ES, m/z): 199[M+H]⁺.

### Step 2: Synthesis of 2-(3-bromophenyl)-5,6,7,8-tetrahydroquinazoline-4-carboxylic acid

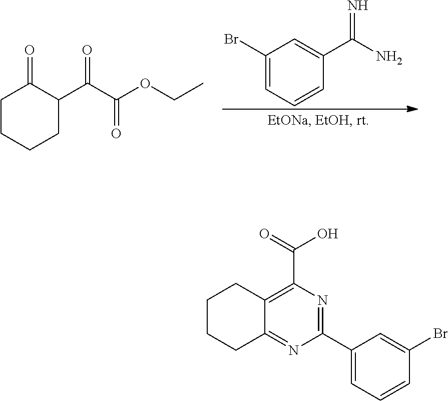 [SMILES: CCOC(=O)C(=O)C1CCCCC1=O ; N=C(N)c1cccc(Br)c1 ; O=C(O)c1nc(-c2cccc(Br)c2)nc2c1CCCC2]

A solution of ethyl 2-oxo-2-(2-oxocyclohexyl)acetate (4.4 g, 22.20 mmol, 1.00 equiv), 3-bromobenzene-1-carboximidamide hydrochloride (5 g, 21.23 mmol, 1.00 equiv) and sodium ethoxide (5 g, 73.48 mmol, 3.30 equiv) in ethanol (120 mL, 2.07 mol, 93.10 equiv) was stirred overnight at room temperature. The reaction was quenched with saturated aqueous ammonium chloride and washed with ethyl ether. The aqueous layer's pH value was adjusted to 3 with 1N hydrogen chloride. The reaction mixture was extracted with ethyl acetate, dried over anhydrous sodium sulfate, and concentrated under vacuum to give 3.6 g (39%) of the title compound as yellow oil which was used for the next step without further purification. LC-MS (ES, m/z): 333, 335[M+H]⁺.

### Step 3: Synthesis of 2-(3-bromophenyl)-5,6,7,8-tetrahydroquinazoline-4-carboxamide

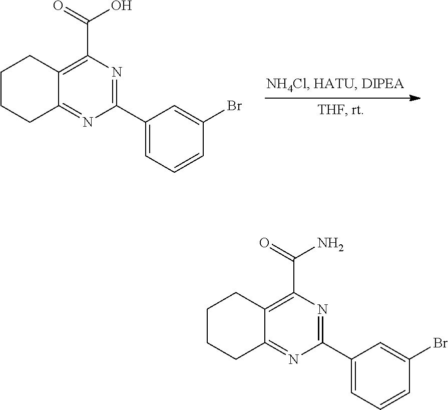 [SMILES: O=C(O)c1nc(-c2cccc(Br)c2)nc2c1CCCC2 ; NC(=O)c1nc(-c2cccc(Br)c2)nc2c1CCCC2]

Similar to as described in General Procedure B, 2-(3-bromophenyl)-5,6,7,8-tetrahydroquinazoline-4-carboxylic acid was reacted with ammonium chloride to give the title compound (600 mg, 60%) as a yellow solid. LC-MS (ES, m/z): 332,334[M+H]⁺.

### Step 4: Synthesis of 2-(3-[2-[(3R)-3-hydroxy-1-methyl-2-oxopyrrolidin-3-yl]ethynyl]phenyl)-5,6,7,8-tetrahydro quinazoline-4-carboxamide

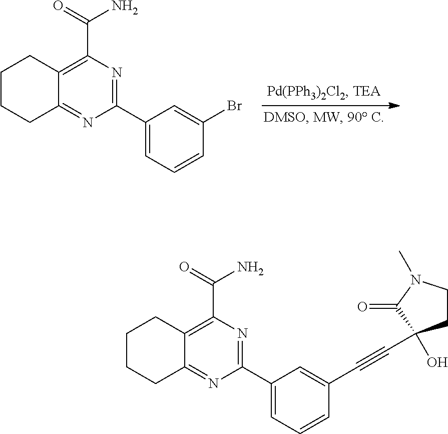 [SMILES: NC(=O)c1nc(-c2cccc(Br)c2)nc2c1CCCC2 ; CN1CC[C@@](O)(C#Cc2cccc(-c3nc4c(c(C(N)=O)n3)CCCC4)c2)C1=O]

Similar to as described in General Procedure E, 2-(3-bromophenyl)-5,6,7,8-tetrahydroquinazoline-4-carboxamide was reacted with (3R)-3-ethynyl-3-hydroxy-1-methylpyrrolidin-2-one to give the title compound (15 mg, 6%) as a white solid. LC-MS (ES, m/z): 391 [M+H]⁺. ¹H NMR (300 MHz, CD₃OD) δ 8.55 (s, 1H), 8.47 (d, J=8.1 Hz, 1H), 7.59-7.48 (m, 2H), 3.52-3.47 (m, 2H), 3.14 (t, J=6.3 Hz, 2H), 3.05 (t, J=6.3 Hz, 2H), 2.95 (s, 3H), 2.66-2.58 (m, 1H), 2.38-2.28 (m, 1H), 2.00-1.88 (m, 4H).

General Procedure X:

Suzuki coupling of boronic acids or boronic esters with aryl halides.

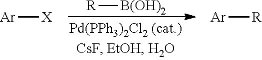

Aryl halide, palladium (II) bis(triphenylphosphine) dichloride or tetrakis (triphenylphosphine) palladium (0.05 eq), boronic acid or pinacol ester (1.1 eq) and cesium fluoride (2 eq) were weighed out into a microwave vessel or sealed tube. Ethanol (3 mL/mmol) and water (0.6 mL/mmol) were added. The vessel was capped and heated thermally or in a microwave vessel at 70˜100° C. for 1 hour. The reaction mixture was concentrated under vacuum and the residue was purified by silica gel column chromatography to afford the Suzuki coupling product.

### Example SS

### Synthesis of (R)-4-(3-((3-hydroxy-1-methyl-2-oxopyrrolidin-3-yl)ethynyl)phenyl)-1-methyl-1H-imidazo[4,5-c]pyridine-6-carboxamide

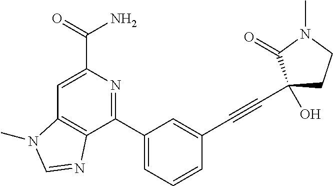 [SMILES: CN1CC[C@@](O)(C#Cc2cccc(-c3nc(C(N)=O)cc4c3ncn4C)c2)C1=O]

### Step 1: Synthesis of 2,6-dichloro-N-methylpyridin-4-amine

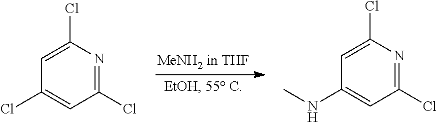

Similar to as described in General Procedure A, 2,4,6-trichloropyridine was reacted with methyl amine to give the title compound (700 mg, 36%) as a white solid. LC-MS (ES, m/z): 177 [M+H]⁺.

### Step 2: Synthesis of, 6-dichloro-N-methyl-3-nitropyridin-4-amine

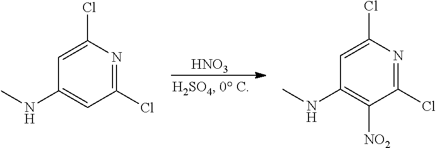

Into a 25-mL round-bottom flask was placed 2,6-dichloro-N-methylpyridin-4-amine (600 mg, 3.39 mmol, 1.00 equiv), nitric acid (1 mL, 22.30 mmol, 6.58 equiv), and sulfuric acid (5 mL, 93.80 mmol, 27.68 equiv) at 0° C. After 1 h the resulting solution was mixed with ice/water, extracted with ethyl acetate, and washed with aqueous sodium carbonate and brine. The organic was concentrated under vacuum and the residue was dissolved in 5 mL of sulfuric acid. The resulting solution was stirred overnight at room temperature and poured slowly into 10 mL of ice/water. The solids were collected by filtration. This resulted in the title compound (600 mg, 80%) as a yellow solid. LC-MS (ES, m/z): 222 [M+H]⁺.

### Step 3: Synthesis of 2,6-dichloro-4-N-methylpyridine-3,4-diamine

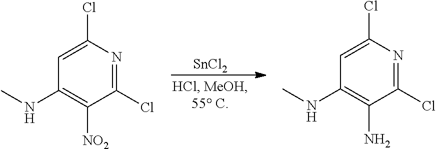

Into a 50-mL round-bottom flask was placed 2,6-dichloro-N-methyl-3-nitropyridin-4-amine (300.0 mg, 1.35 mmol, 1.00 equiv), tin(III)chloride (1.02 g, 5.40 mmol, 4.00 equiv), methanol (20 mL), and concentrated hydrogen chloride (2.5 mL). The resulting solution was stirred for 1 h at 55° C. The pH value of the solution was adjusted to 8 with sodium hydroxide (1 M). The resulting solution was extracted with ethyl acetate, washed with water, dried over anhydrous sodium sulfate, and concentrated under vacuum. This resulted in the title compound (250 mg, 96%) as a brown solid. LC-MS (ES, m/z): 192 [M+H]⁺.

### Step 4: Synthesis of 4,6-dichloro-1-methyl-1H-imidazo[4,5-c]pyridine

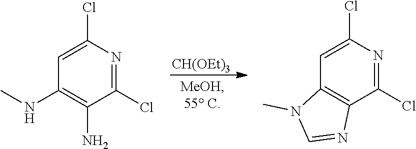

A solution of 2,6-dichloro-4-N-methylpyridine-3,4-diamine (250 mg, 1.30 mmol, 1.00 equiv) and triethyl orthoformate (5 mL) in methanol (10 mL) was stirred for 16 h at 55° C. The reaction was quenched by water. The pH value of the solution was adjusted to 9 with sodium hydroxide (1 M). The resulting solution was extracted with ethyl acetate, washed with water and brine, dried over anhydrous sodium sulfate, and concentrated under vacuum. This resulted in 220 mg (84%) of the title compound as a brown solid. LC-MS (ES, m/z): 202 [M+H]⁺.

### Step 5: Synthesis of (R)-3-((3-(6-chloro-1-methyl-1H-imidazo[4,5-c]pyridin-4-yl)phenyl)ethynyl)-3-hydroxy-1-methylpyrrolidin-2-one

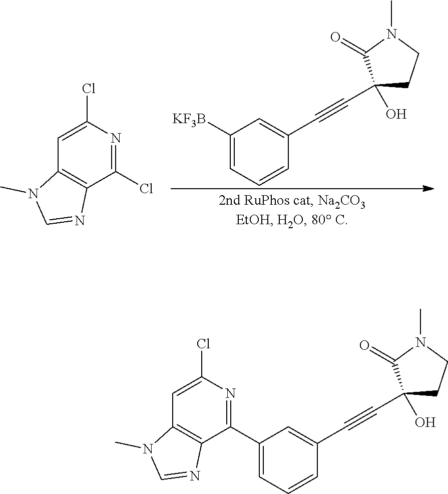

Similar to as described in General Procedure U, 4,6-dichloro-1-methyl-1H-imidazo[4,5-c]pyridine was reacted with potassium (R)-trifluoro(3-((3-hydroxy-1-methyl-2-oxopyrrolidin-3-yl)ethynyl)phenyl)borate to give the title compound (80 mg, 20%) as a yellow solid. LC-MS (ES, m/z): 381 [M+H]⁺.

### Step 6: Synthesis of methyl 4-(3-[2-[(3R)-3-hydroxy-1-methyl-2-oxopyrrolidin-3-yl]ethynyl]phenyl)-1-methyl-1H-imidazo[4,5-c]pyridine-6-carboxylate

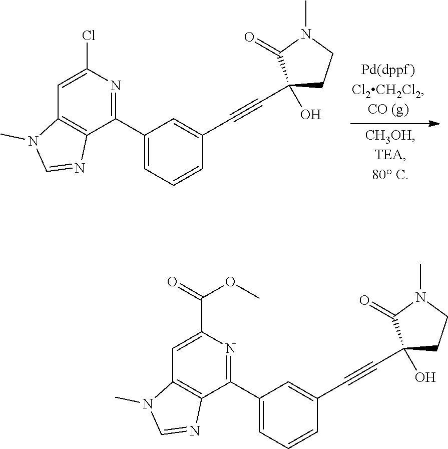 [SMILES: CN1CC[C@@](O)(C#Cc2cccc(-c3nc(Cl)cc4c3ncn4C)c2)C1=O ; COC(=O)c1cc2c(ncn2C)c(-c2cccc(C#C[C@]3(O)CCN(C)C3=O)c2)n1]

Similar to as described in General Procedure O, (3R)-3-[2-(3-[6-chloro-1-methyl-1H-imidazo[4,5-c]pyridin-4-yl]phenyl)ethynyl]-3-hydroxy-1-methylpyrrolidin-2-one was reacted with carbon monoxide to give the title compound (10 mg, 12%) as an off-white solid. LC-MS (ES, m/z): 405 [M+H]⁺.

### Step 7: Synthesis of 4-(3-[2-[(3R)-3-hydroxy-1-methyl-2-oxopyrrolidin-3-yl]ethynyl]phenyl)-1-methyl-1H-imidazo[4,5-c]pyridine-6-carboxamide

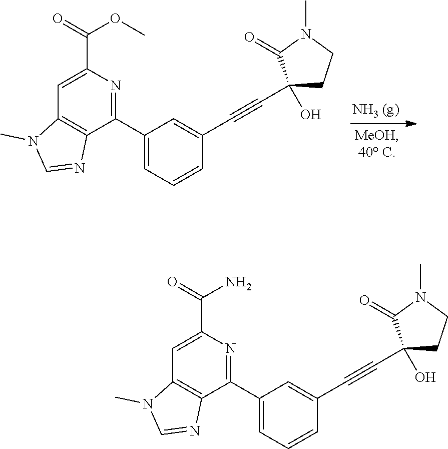 [SMILES: COC(=O)c1cc2c(ncn2C)c(-c2cccc(C#C[C@]3(O)CCN(C)C3=O)c2)n1 ; CN1CC[C@@](O)(C#Cc2cccc(-c3nc(C(N)=O)cc4c3ncn4C)c2)C1=O]

Similar to as described in General Procedure S, methyl 4-(3-[2-[(3R)-3-hydroxy-1-methyl-2-oxopyrrolidin-3-yl]ethynyl]phenyl)-1-methyl-1H-imidazo[4,5-c]pyridine-6-carboxylate was reacted with ammonia to give the title compound (5.5 mg, 29%) as an off-white solid. LC-MS (ES, m/z): 390 [M+H]⁺. ¹H NMR (300 MHz, CD₃OD) δ 8.77 (s, 1H), 8.77-8.65 (m, 1H), 8.41 (s, 1H), 8.35 (s, 1H), 7.60-7.52 (m, 2H), 4.03 (s, 3H), 3.56-3.47 (m, 2H), 2.96 (s, 3H), 2.68-2.61 (m, 1H), 2.40-2.30 (m, 1H).

### Example TT

### Synthesis of (R)-8-(3-((3-hydroxy-1-methyl-2-oxopyrrolidin-3-yl)ethynyl)phenyl)imidazo[1,2-a]pyrazine-6-carboxamide

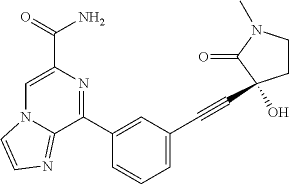 [SMILES: CN1CC[C@@](O)(C#Cc2cccc(-c3nc(C(N)=O)cn4ccnc34)c2)C1=O]

### Step 1: Synthesis of methyl 5-amino-6-bromopyrazine-2-carboxylate

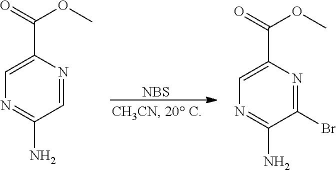

A solution of methyl 5-aminopyrazine-2-carboxylate (2.852 g, 18.62 mmol, 1.00 equiv) and NBS (3.98 g, 22.36 mmol, 1.20 equiv) in acetonitrile (30 mL) was stirred for 12 h at 20° C. The resulting mixture was concentrated under vacuum and the residue was purified by a silica gel column with ethyl acetate/petroleum ether (1:1). The purified product was washed with 30 mL of hot water and dried to give the title compound (1.1 g, 26%) as a light yellow solid. LC-MS (ES, m/z): 232, 234 [M+H]⁺.

### Step 2: Synthesis of methyl 8-bromoimidazo[1,2-a]pyrazine-6-carboxylate

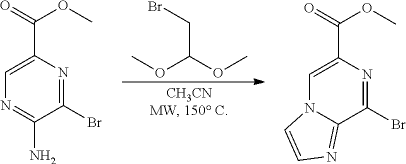

A solution of methyl 5-amino-6-bromopyrazine-2-carboxylate (300 mg, 1.29 mmol, 1.00 equiv) and 2-bromo-1,1-dimethoxyethane (218 mg, 1.29 mmol, 1.00 equiv) in acetonitrile (10 mL) was irradiated with microwave for 2 h at 150° C. The reaction solution was cooled to room temperature and concentrated under vacuum. The residue was purified by a silica gel column with dichloromethane/methanol (10:1) to give the title compound (321 mg, 97%) as a light yellow solid. LC-MS (ES, m/z): 256, 258 [M+H]⁺.

### Step 3: Synthesis of methyl 8-(3-[2-[(3R)-3-hydroxy-1-methyl-2-oxopyrrolidin-3-yl]ethynyl]phenyl)imidazo[1,2-a]pyrazine-6-carboxylate

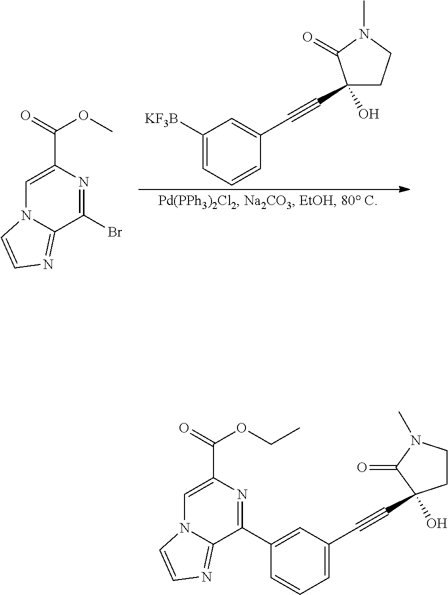

Similar to as described in General Procedure U, methyl 8-bromoimidazo[1,2-a]pyrazine-6-carboxylate was reacted with potassium (R)-trifluoro(3-((3-hydroxy-1-methyl-2-oxopyrrolidin-3-yl)ethynyl)phenyl)borate to give the title compound (205 mg, 34%) as a light yellow solid. LC-MS (ES, m/z): 405 [M+H]⁺.

### Step 4: Synthesis of 8-(3-[2-[(3R)-3-hydroxy-1-methyl-2-oxopyrrolidin-3-yl]ethynyl]phenyl)imidazo[1,2-a]pyrazine-6-carboxamide

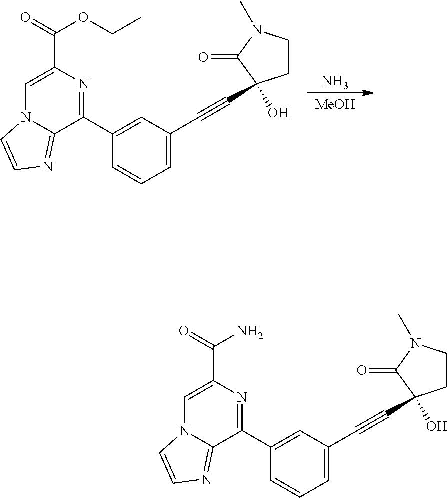

Similar to as described in General Procedure S, 8-(3-[2-[(3R)-3-hydroxy-1-methyl-2-oxopyrrolidin-3-yl]ethynyl]phenyl)imidazo[1,2-a]pyrazine-6-carboxylate was reacted with ammonia to give the title compound (114.5 mg, 41%) as an off-white solid. LC-MS (ES, m/z): 376 [M+H]⁺. ¹H NMR (400 MHz, CD₃OD) δ 9.19 (s, 1H), 8.87 (s, 1H), 8.75 (d, J=8.0 Hz, 1H), 8.24 (s, 1H), 7.93 (s, 1H), 7.67-7.56 (m, 2H), 3.56-3.46 (m, 2H), 2.96 (s, 3H), 2.67-2.61 (m, 1H), 2.38-2.31 (m, 1H).

### Example UU

### Synthesis of (R)-5-(3-((3-hydroxy-1-methyl-2-oxopyrrolidin-3-yl)ethynyl)phenyl)-1H-pyrrolo[2,3-c]pyridine-7-carboxamide

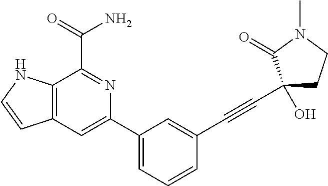 [SMILES: CN1CC[C@@](O)(C#Cc2cccc(-c3cc4cc[nH]c4c(C(N)=O)n3)c2)C1=O]

### Step 1: Synthesis of 2-bromo-6-chloro-3-nitropyridine

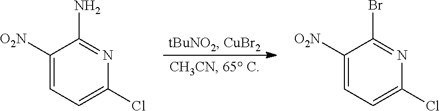 [SMILES: Nc1nc(Cl)ccc1[N+](=O)[O-] ; O=[N+]([O-])c1ccc(Cl)nc1Br]

t-Butyl nitrite (8.5 g, 82.43 mmol, 1.80 equiv) was added to a mixture of 6-chloro-3-nitropyridin-2-amine (8 g, 46.09 mmol, 1.00 equiv) and copper(II) bromide (12.3 g, 55.07 mmol, 1.20 equiv) in acetonitrile (120 mL, 2.28 mol) under nitrogen. The resulting mixture was stirred for 30 min at 65° C. and partitioned between ethyl acetate and 2 M aqueous hydrochloric acid. The organic layer was dried over anhydrous sodium sulfate and concentrated under vacuum. The residue was purified by silica gel column chromatography eluting with ethyl acetate/petroleum ether (1:5). This resulted in the title compound (8.2 g, 75%) as a yellow solid.

### Step 2: Synthesis of 7-bromo-5-chloro-1H-pyrrolo[2,3-c]pyridine

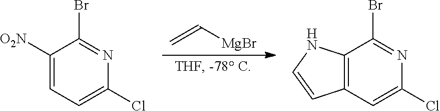

Under nitrogen a solution of 2-bromo-6-chloro-3-nitropyridine (2.5 g, 10.53 mmol, 1.00 equiv) in THF (60 mL) was cooled to −78° C. and bromo(ethenyl)magnesium (1M in THF, 63 mL, 6 equiv) was added dropwise. The reaction was stirred for 1 h at −50° C., quenched with aqueous ammonium chloride, extracted with ethyl acetate, dried over sodium sulfate, and concentrated in vacuum. The residue was purified by silica gel column chromatography eluting with ethyl acetate/petroleum ether (1:3). This resulted in the title compound (1.3 g, 53%) as a yellow solid. LC-MS (ES, m/z): 231 [M+H]⁺.

### Step 3: Synthesis of methyl 5-chloro-1H-pyrrolo[2,3-c]pyridine-7-carboxylate

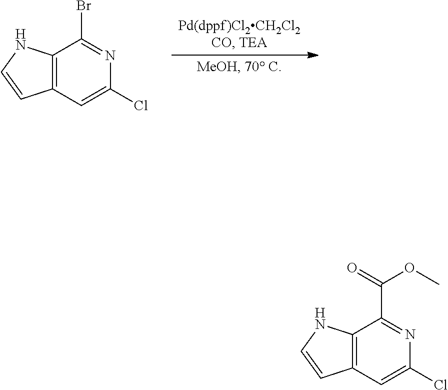

Similar to as described in General Procedure O, 7-bromo-5-chloro-1H-pyrrolo[2,3-c]pyridine was reacted with carbon monoxide to afford the title compound (380 mg, 84%) as a white solid. LC-MS (ES, m/z): 211 [M+H]⁺.

### Step 4: Synthesis of methyl 5-chloro-1-[(4-methylbenzene)sulfonyl]-1H-pyrrolo[2,3-c]pyridine-7-carboxylate

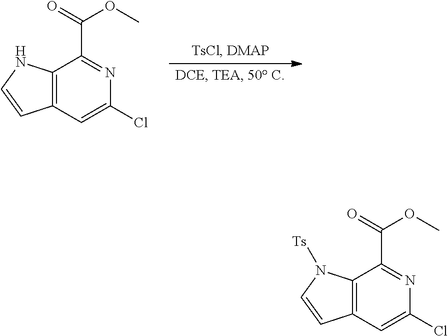 [SMILES: COC(=O)c1nc(Cl)cc2cc[nH]c12 ; COC(=O)c1nc(Cl)cc2ccn(S(=O)(=O)c3ccc(C)cc3)c12]

A solution of methyl 5-chloro-1H-pyrrolo[2,3-c]pyridine-7-carboxylate (420 mg, 1.99 mmol, 1.00 equiv), 4-methylbenzene-1-sulfonyl chloride (570 mg, 2.99 mmol, 1.50 equiv), 4-dimethylaminopyridine (25 mg, 0.20 mmol, 0.10 equiv) in dichloroethane (10 mL) and triethylamine (8 mL) was stirred for 5 h at 50° C. The mixture was concentrated under vacuum and the residue was purified by silica gel column chromatography eluting with ethyl acetate/petroleum ether (1:3) to afford the title compound (400 mg, 55%) as a yellow solid. LC-MS (ES, m/z): 365 [M+H]⁺.

### Step 5: Synthesis of methyl 5-(3-[2-[(3R)-3-hydroxy-1-methyl-2-oxopyrrolidin-3-yl]ethynyl]phenyl)-1-[(4-methylbenzene)sulfonyl]-1H-pyrrolo[2,3-c]pyridine-7-carboxylate

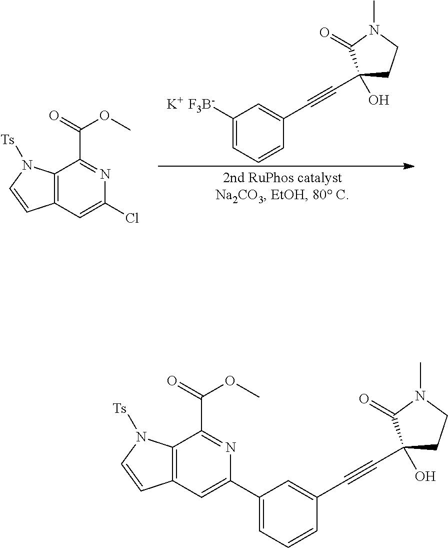

Similar to as described in General Procedure U, methyl 5-chloro-1-[(4-methylbenzene) sulfonyl]-1H-pyrrolo[2,3-c]pyridine-7-carboxylate was reacted with potassium (R)-trifluoro(3-((3-hydroxy-1-methyl-2-oxopyrrolidin-3-yl)ethynyl)phenyl)borate to give the title compound (6 mg, 40%) as yellow oil. LC-MS (ES, m/z): 544 [M+H]⁺.

### Step 6: Synthesis of 5-(3-[2-[(3R)-3-hydroxy-1-methyl-2-oxopyrrolidin-3-yl]ethynyl]phenyl)-1H-pyrrolo[2,3-c]pyridine-7-carboxamide

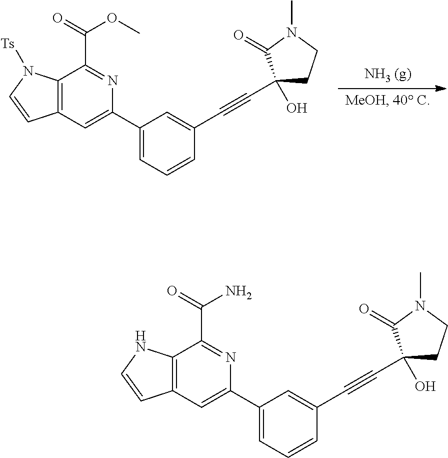 [SMILES: COC(=O)c1nc(-c2cccc(C#C[C@]3(O)CCN(C)C3=O)c2)cc2ccn(S(=O)(=O)c3ccc(C)cc3)c12 ; CN1CC[C@@](O)(C#Cc2cccc(-c3cc4cc[nH]c4c(C(N)=O)n3)c2)C1=O]

Similar to as described in General Procedure S, 5-(3-[2-[(3R)-3-hydroxy-1-methyl-2-oxopyrrolidin-3-yl]ethynyl]phenyl)-1-[(4-methylbenzene)sulfonyl]-1H-pyrrolo[2,3-c]pyridine-7-carboxylate was reacted with ammonia to give the title compound (39.6 mg, 19%) as a white solid. LC-MS (ES, m/z): 375 [M+H]⁺. ¹H NMR (400 MHz, DMSO-d₆) δ 11.60 (s, 1H), 8.39-8.32 (m, 3H), 8.26 (s, 1H), 7.77 (s, 1H), 7.61-7.60 (m, 1H), 7.50-7.46 (m, 1H), 7.20-7.40 (m, 1H), 6.63 (d, J=2.0 Hz, 1H), 3.39-3.36 (m, 2H), 2.82 (s, 3H), 2.50-2.45 (m, 1H), 2.24-2.19 (m, 1H)

### Example VV

### Synthesis of (R)-4-(3-((3-hydroxy-1-methyl-2-oxopyrrolidin-3-yl)ethynyl)phenyl)-7-methyl-7H-pyrrolo[2,3-d]pyrimidine-2-carboxamide

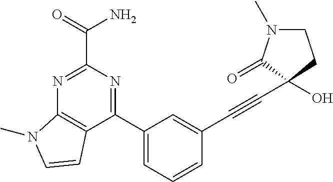 [SMILES: CN1CC[C@@](O)(C#Cc2cccc(-c3nc(C(N)=O)nc4c3ccn4C)c2)C1=O]

### Step 1: Synthesis of 2,4-dichloro-7-methyl-7H-pyrrolo[2,3-d]pyrimidine

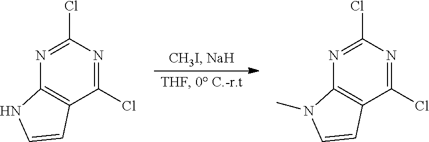

2,4-Dichloro-7H-pyrrolo[2,3-d]pyrimidine (1.053 g, 5.60 mmol, 1.00 equiv) was added dropwise to a stirred solution of sodium hydride (256.9 mg, 10.71 mmol, 1.90 equiv) in THF (60 mL) at 0° C. The result solution was stirred for another 30 min and then methyl iodide (1.2723 g, 8.96 mmol, 1.60 equiv) was added dropwise at 0° C. The resulting solution was stirred for 12 h at 25° C., quenched with water, extracted with ethyl acetate, washed with brine, dried over anhydrous sodium sulfate, and concentrated under vacuum. The residue was purified by a silica gel column with ethyl acetate/petroleum ether (1:5) to give the title compound (1.042 g (92%) as an off-white solid. LC-MS (ES, m/z): 202, 204 [M+H]⁺.

### Step 2: Synthesis of (3R)-3-[2-(3-[2-chloro-7-methyl-7H-pyrrolo[2,3-d]pyrimidin-4-yl]phenyl)ethynyl]-3-hydroxy-1-methylpyrrolidin-2-one

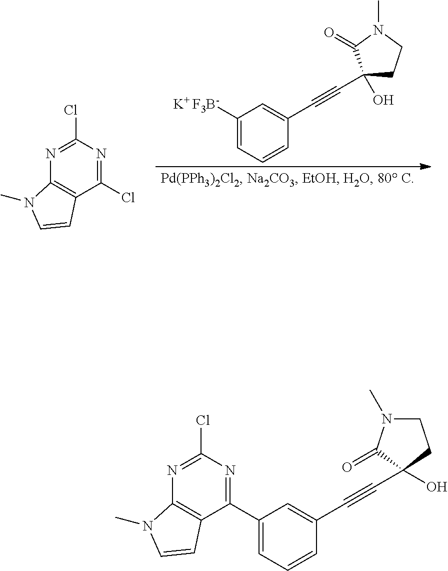

Similar to as described in General Procedure U, 2,4-dichloro-7-methyl-7H-pyrrolo[2,3-d]pyrimidine was reacted with potassium trifluoro(3-2-[(3R)-3-hydroxy-1-methyl-2-oxopyrrolidin-3-yl]ethynylphenyl)boranideto give 422 mg (crude) of the title compound as a light yellow solid. LC-MS (ES, m/z): 381, 383 [M+H]⁺.

### Step 3: Synthesis of 4-(3-[2-[(3R)-3-hydroxy-1-methyl-2-oxopyrrolidin-3-yl]ethynyl]phenyl)-7-methyl-7H-pyrrolo[2,3-d]pyrimidine-2-carboxamide

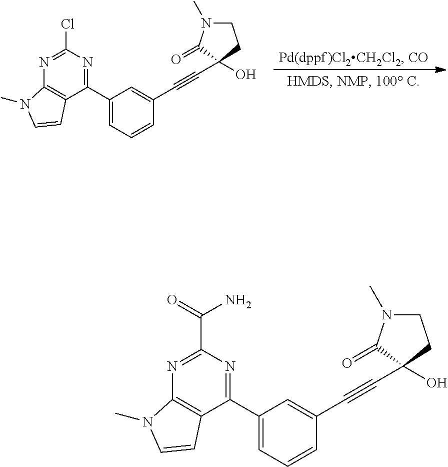 [SMILES: CN1CC[C@@](O)(C#Cc2cccc(-c3nc(Cl)nc4c3ccn4C)c2)C1=O ; CN1CC[C@@](O)(C#Cc2cccc(-c3nc(C(N)=O)nc4c3ccn4C)c2)C1=O]

Similar to as described in General Procedure S, (3R)-3-[2-(3-[2-chloro-7-methyl-7H-pyrrolo[2,3-d]pyrimidin-4-yl]phenyl)ethynyl]-3-hydroxy-1-methylpyrrolidin-2-one was reacted with carbon monoxide to give the title compound (21.6 mg, 14%) as an off-white solid. LC-MS (ES, m/z): 390 [M+H]⁺. ¹H NMR (400 MHz, CD₃OD) δ 8.38 (s, 1H), 8.29 (d, J=7.6 Hz, 1H), 7.74 (d, J=4.0 Hz, 1H), 7.67-7.59 (m, 2H), 7.01 (d, J=3.2 Hz, 1H), 4.03 (s, 3H), 3.56-3.44 (m, 2H), 2.96 (s, 3H), 2.67-2.61 (m, 1H), 2.38-2.31 (m, 1H).

### Example WW

### Synthesis of (R)-6-(3-((3-hydroxy-1-methyl-2-oxopyrrolidin-3-yl)ethynyl)phenyl)-4-(1H-1,2,4-triazol-1-yl)picolinamide

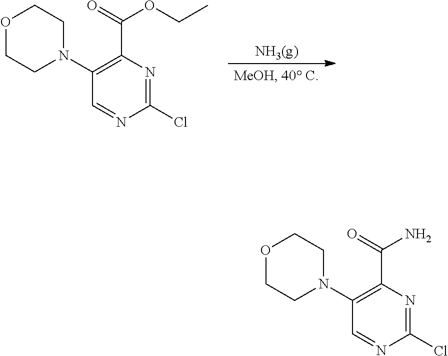 [SMILES: CCOC(=O)c1nc(Cl)ncc1N1CCOCC1 ; NC(=O)c1nc(Cl)ncc1N1CCOCC1]

### Step 1: Synthesis of 2,6-dichloro-4-(1H-1,2,4-triazol-1-yl)pyridine

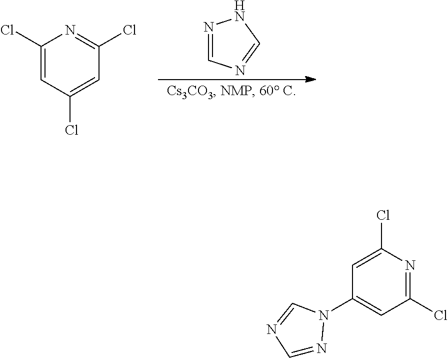 [SMILES: Clc1cc(Cl)nc(Cl)c1 ; c1nc[nH]n1 ; Clc1cc(-n2cncn2)cc(Cl)n1]

A mixture of 2,4,6-trichloropyridine (1 g, 5.48 mmol, 1.00 equiv), Cs₂CO₃ (2.4 g, 7.37 mmol, 1.30 equiv), 1-methyl-2-pyrrolidinone (4 mL), and 1H-1,2,4-triazole (373 mg, 5.40 mmol, 1.00 equiv) was stirred for 4 h at 60° C. The resulting solution was diluted with 150 mL of ethyl acetate, washed with brine, dried over anhydrous sodium sulfate, and concentrated under vacuum. The residue was applied onto a silica gel column with ethyl acetate/petroleum ether (1:5) to give the title compound (495 mg, 42%) as a white solid. LC-MS (ES, m/z): 215 [M+H]⁺.

### Step 2: Synthesis of (3R)-3-(2-[3-[6-chloro-4-(1H-1,2,4-triazol-1-yl)pyridin-2-yl]phenyl]ethynyl)-3-hydroxy-1-methylpyrrolidin-2-one

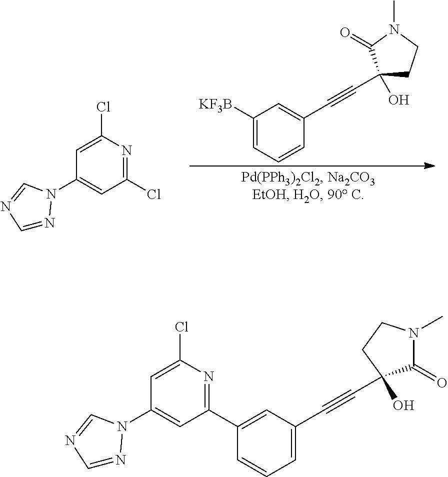

Similar to as described in General Procedure U, 2,6-dichloro-4-(1H-1,2,4-triazol-1-yl)pyridine was reacted with potassium trifluoro(3-2-[(3R)-3-hydroxy-1-methyl-2-oxopyrrolidin-3-yl]ethynylphenyl)boranideto give the title compound (185 mg, 37%) as a yellow solid. LC-MS (ES, m/z): 394 [M+H]⁺.

### Step 3: Synthesis of ethyl 6-(3-[2-[(3R)-3-hydroxy-1-methyl-2-oxopyrrolidin-3-yl]ethynyl]phenyl)-4-(1H-1,2,4-triazol-1-yl)pyridine-2-carboxylate

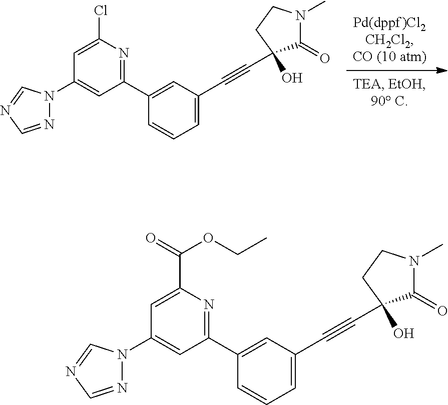 [SMILES: CN1CC[C@@](O)(C#Cc2cccc(-c3cc(-n4cncn4)cc(Cl)n3)c2)C1=O ; CCOC(=O)c1cc(-n2cncn2)cc(-c2cccc(C#C[C@]3(O)CCN(C)C3=O)c2)n1]

Similar to as described in General Procedure O, (3R)-3-(2-[3-[6-chloro-4-(1H-1,2,4-triazol-1-yl)pyridin-2-yl]phenyl]ethynyl)-3-hydroxy-1-methylpyrrolidin-2-one was reacted with carbon monoxide to give the title compound (95 mg, 48%) as an off-white solid. LC-MS (ES, m/z): 432 [M+H]⁺.

### Step 4: Synthesis of

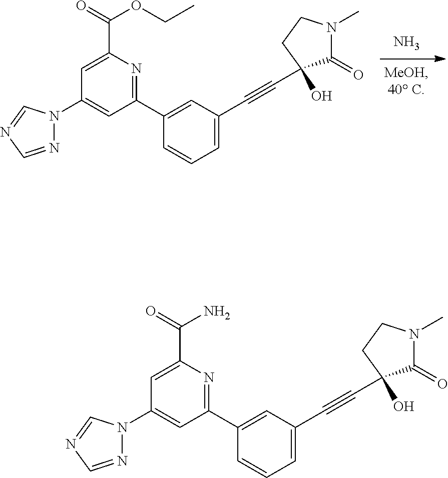

Similar to as described in General Procedure S, ethyl 6-(3-[2-[(3R)-3-hydroxy-1-methyl-2-oxopyrrolidin-3-yl]ethynyl]phenyl)-4-(1H-1,2,4-triazol-1-yl)pyridine-2-carboxylate was reacted with ammonia to give the title compound (11.7 mg, 16%) as an off-white solid. LC-MS (ES, m/z): 403 [M+H]⁺. ¹H NMR (400 MHz, CD₃OD, ppm): 6:9.57 (s, 1H), 8.62-8.59 (m, 2H), 8.43 (s, 1H), 8.33 (d, J=8.4 Hz, 2H), 7.65-7.55 (m, 2H), 3.56-3.50 (m, 2H), 2.96 (s, 3H), 2.67-2.61 (m, 1H), 2.39-2.32 (m, 1H).

### Example XX

### Synthesis of (R)-6-(3-((3-hydroxy-1-methyl-2-oxopyrrolidin-3-yl)ethynyl)phenyl)-4-(1H-pyrazol-1-yl)picolinamide

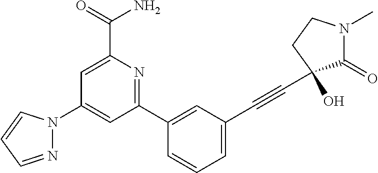 [SMILES: CN1CC[C@@](O)(C#Cc2cccc(-c3cc(-n4cccn4)cc(C(N)=O)n3)c2)C1=O]

### Step 1: Synthesis of 2,6-dichloro-4-(1H-pyrazol-1-yl)pyridine

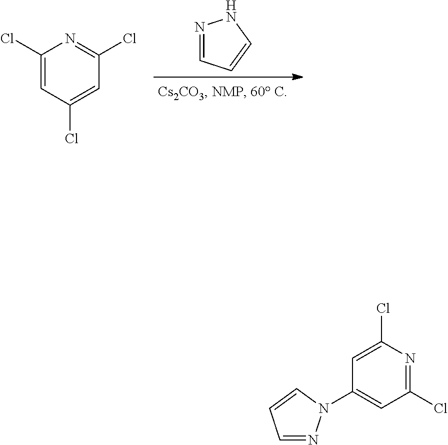 [SMILES: Clc1cc(Cl)nc(Cl)c1 ; c1cn[nH]c1 ; Clc1cc(-n2cccn2)cc(Cl)n1]

A mixture of 2,4,6-trichloropyridine (2 g, 10.96 mmol, 1.00 equiv), cesium carbonate (4.23 g, 12.98 mmol, 1.20 equiv), 1-methyl-2-pyrrolidinone (7 mL), and 1H-pyrazole (740 mg, 10.87 mmol, 1.00 equiv) was stirred for 4 h at 60° C. The resulting solution was diluted with ethyl acetate, washed with brine, extracted with ethyl acetate, and concentrated under vacuum. The residue was applied onto a silica gel column with ethyl acetate/petroleum ether (1:20) to give the title compound (0.98 g, 43%) as an off-white solid. LC-MS (ES, m/z): 214 [M+H]⁺.

### Step 2: Synthesis of (3R)-3-(2-[3-[6-chloro-4-(1H-pyrazol-1-yl)pyridin-2-yl]phenyl]ethynyl)-3-hydroxy-1-methylpyrrolidin-2-one

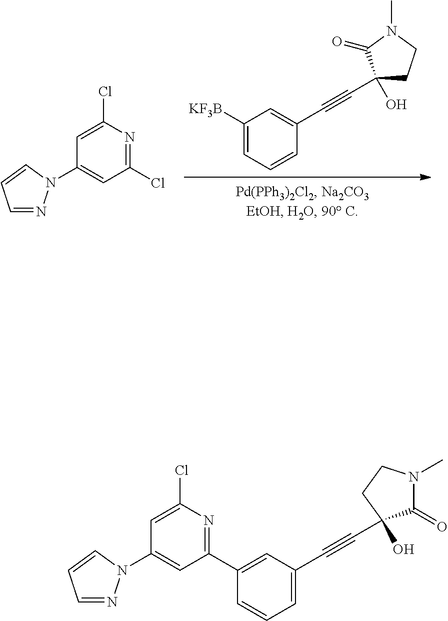

Similar to as described in General Procedure U, 2,6-di chloro-4-(1H-pyrazol-1-yl)pyridine was reacted with potassium trifluoro(3-2-[(3R)-3-hydroxy-1-methyl-2-oxopyrrolidin-3-yl]ethynylphenyl)boranideto give the title compound (176 mg, 32%) as a yellow solid. LC-MS (ES, m/z): 393 [M+H]⁺.

### Step 3: Synthesis of ethyl 6-(3-[2-[(3R)-3-hydroxy-1-methyl-2-oxopyrrolidin-3-yl]ethynyl]phenyl)-4-(1H-pyrazol-1-yl)pyridine-2-carboxylate

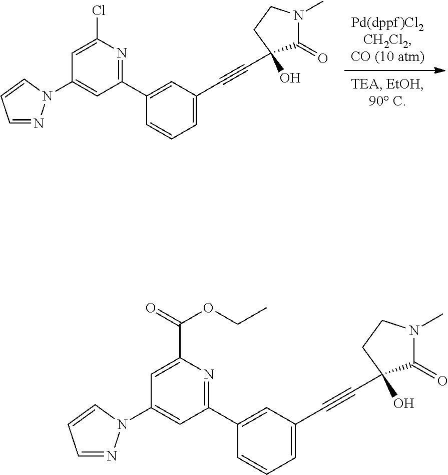 [SMILES: CN1CC[C@@](O)(C#Cc2cccc(-c3cc(-n4cccn4)cc(Cl)n3)c2)C1=O ; CCOC(=O)c1cc(-n2cccn2)cc(-c2cccc(C#C[C@]3(O)CCN(C)C3=O)c2)n1]

Similar to as described in General Procedure O, (3R)-3-(2-[3-[6-chloro-4-(1H-pyrazol-1-yl)pyridin-2-yl]phenyl]ethynyl)-3-hydroxy-1-methylpyrrolidin-2-one was reacted with carbon monoxide to give the title compound (130 mg, 74%) as a dark red solid. LC-MS (ES, m/z): 431 [M+H]⁺.

### Step 4: Synthesis of 6-(3-[2-[(3R)-3-hydroxy-1-methyl-2-oxopyrrolidin-3-yl]ethynyl]phenyl)-4-(1H-pyrazol-1-yl)pyridine-2-carboxamide

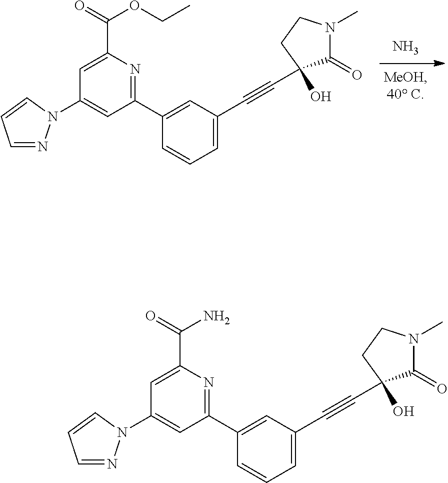

Similar to as described in General Procedure S, ethyl 6-(3-[2-[(3R)-3-hydroxy-1-methyl-2-oxopyrrolidin-3-yl]ethynyl]phenyl)-4-(1H-pyrazol-1-yl)pyridine-2-carboxylate was reacted with ammonia to give the title compound (19.2 mg, 26%) as an off-white solid. LC-MS (ES, m/z): 402 [M+H]⁺. ¹H NMR (300 MHz, CD₃OD) δ 8.63 (s, 1H), 8.49 (s, 2H), 8.37 (s, 1H), 8.27 (d, J=6.6 Hz, 2H), 7.86 (s, 1H), 7.56-7.53 (d, 3H, J=11.4 Hz), 6.65 (s, 1H), 3.49 (d, 2H, J=4.5 Hz), 2.94 (s, 3H), 2.64-2.60 (m, 1H), 2.37-2.28 (m, 1H).

### Example YY

### Synthesis of (R)-4-(3-((3-hydroxy-1-methyl-2-oxopyrrolidin-3-yl)ethynyl)phenyl)-6-(1-methyl-1H-pyrazol-5-yl)pyrimidine-2-carboxamide

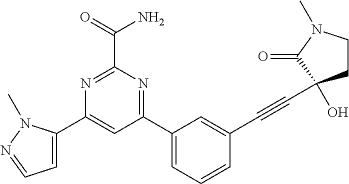 [SMILES: CN1CC[C@@](O)(C#Cc2cccc(-c3cc(-c4ccnn4C)nc(C(N)=O)n3)c2)C1=O]

### Step 1: Synthesis of 2,4-dichloro-6-(1-methyl-1H-pyrazol-5-yl)pyrimidine

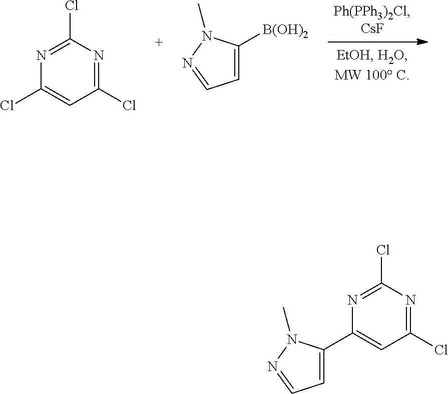

Similar to as described in General Procedure X, 2,4,6-trichloropyrimidine was reacted with (1-methyl-1H-pyrazol-5-yl)boronic acid to give the title compound (256 mg, 51%) as an off-white solid. LC-MS (ES, m/z): 229 [M+H]⁺.

### Step 2: Synthesis of (3R)-3-(2-[3-[2-chloro-6-(1-methyl-1H-pyrazol-5-yl)pyrimidin-4-yl]phenyl]ethynyl)-3-hydroxy-1-methylpyrrolidin-2-one

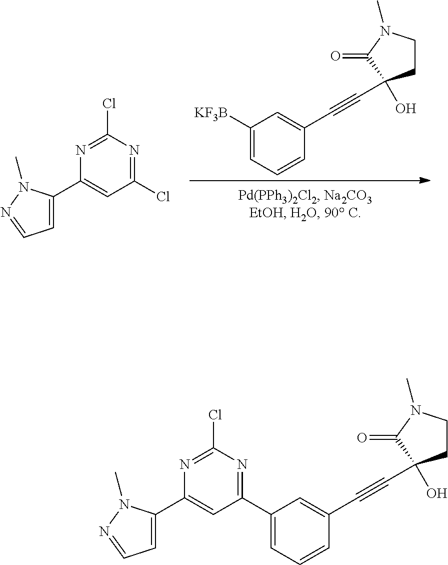

Similar to as described in General Procedure U, 2,4-dichloro-6-(1-methyl-1H-pyrazol-5-yl)pyrimidine was reacted with potassium trifluoro(3-2-[(3R)-3-hydroxy-1-methyl-2-oxopyrrolidin-3-yl]ethynylphenyl)boranideto give the title compound (180 mg, 67%) as a light yellow solid. LC-MS (ES, m/z): 408 [M+H]⁺.

### Step 3: Synthesis of methyl 4-(3-[2-[(3R)-3-hydroxy-1-methyl-2-oxopyrrolidin-3-yl]ethynyl]phenyl)-6-(1-methyl-1H-pyrazol-5-yl)pyrimidine-2-carboxylate

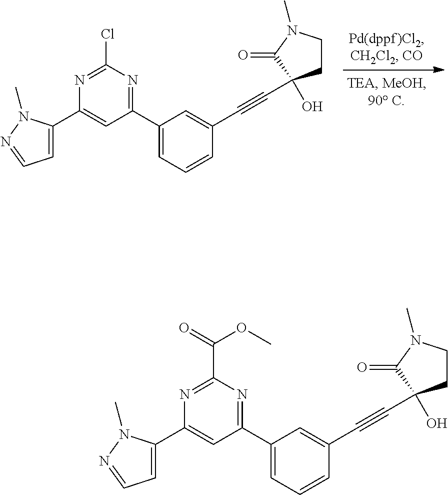 [SMILES: CN1CC[C@@](O)(C#Cc2cccc(-c3cc(-c4ccnn4C)nc(Cl)n3)c2)C1=O ; COC(=O)c1nc(-c2cccc(C#C[C@]3(O)CCN(C)C3=O)c2)cc(-c2ccnn2C)n1]

Similar to as described in General Procedure O, (3R)-3-(2-[3-[2-chloro-6-(1-methyl-1H-pyrazol-5-yl)pyrimidin-4-yl]phenyl]ethynyl)-3-hydroxy-1-methylpyrrolidin-2-one was reacted with carbon monoxide to give the title compound (123 mg, 73%) as a light yellow solid. LC-MS (ES, m/z): 432 [M+H]⁺.

### Step 4: Synthesis of 4-(3-[2-[(3R)-3-hydroxy-1-methyl-2-oxopyrrolidin-3-yl]ethynyl]phenyl)-6-(1-methyl-1H-pyrazol-5-yl)pyrimidine-2-carboxamide

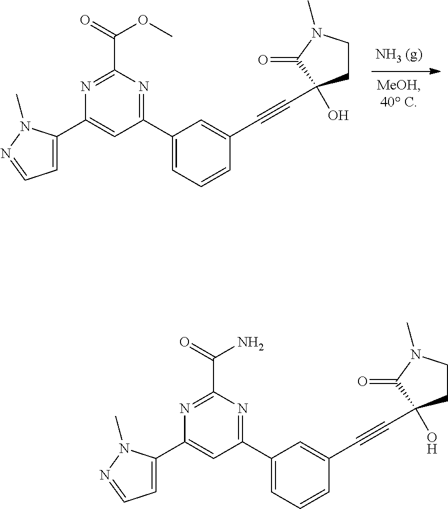 [SMILES: COC(=O)c1nc(-c2cccc(C#C[C@]3(O)CCN(C)C3=O)c2)cc(-c2ccnn2C)n1 ; CN1CC[C@@](O)(C#Cc2cccc(-c3cc(-c4ccnn4C)nc(C(N)=O)n3)c2)C1=O]

Similar to as described in General Procedure S, methyl 4-(3-[2-[(3R)-3-hydroxy-1-methyl-2-oxopyrrolidin-3-yl]ethynyl]phenyl)-6-(1-methyl-1H-pyrazol-5-yl)pyrimidine-2-carboxylate was reacted with ammonia to give the title compound (20 mg, 41%) as a white solid. LC-MS (ES, m/z): 417 [M+H]⁺. ¹H NMR (300 MHz, CD₃OD) δ 8.53 (s, 1H), 8.43-8.39 (m, 2H), 7.68-7.66 (m, 1H), 7.61-7.58 (m, 2H), 7.22 (d, J=3.0 Hz, 1H), 4.38 (s, 3H), 3.53-3.48 (m, 2H), 2.95 (s, 3H), 2.67-2.59 (m, 1H), 2.39-2.30 (m, 1H).

### Example ZZ

### Synthesis of (S)-ethyl 2-(3-(3-hydroxy-3-(5-methyloxazol-2-yl)but-1-ynyl)phenyl)-7-methyl-7H-pyrrolo[2,3-d]pyrimidine-4-carboxylate

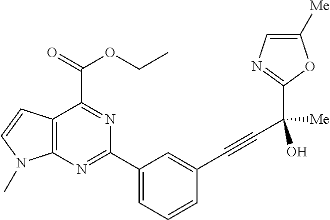 [SMILES: CCOC(=O)c1nc(-c2cccc(C#C[C@](C)(O)c3ncc(C)o3)c2)nc2c1ccn2C]

### Step 1: Synthesis of (9-ethyl 2-(3-(3-hydroxy-3-(5-methyloxazol-2-yl)but-1-ynyl)phenyl)-7-methyl-7H-pyrrolo[2,3-d]pyrimidine-4-carboxylate

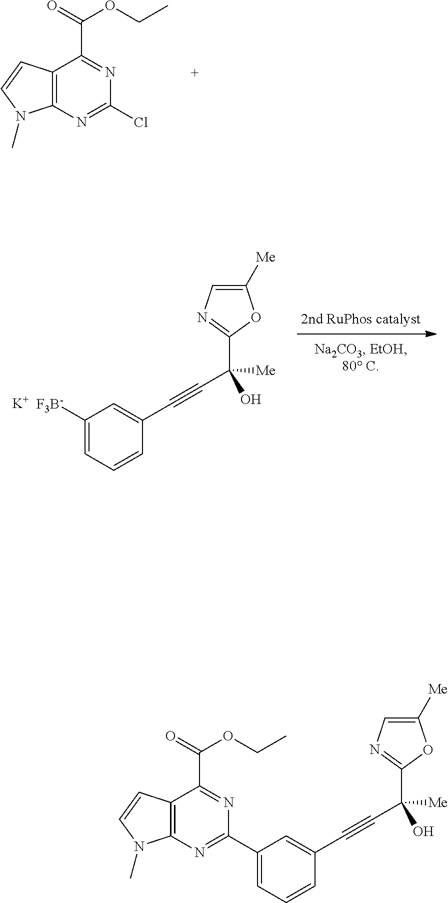

Similar to as described in General Procedure U, ethyl 2-chloro-7-methyl-7H-pyrrolo[2,3-d]pyrimidine-4-carboxylate was reacted with potassium trifluoro(3-2-[(3R)-3-hydroxy-1-methyl-2-oxopyrrolidin-3-yl]ethynylphenyl)boranideto give the title compound (31 mg, 12%) as yellow oil. LC-MS (ES, m/z): 431 [M+H]⁺.

### Step 2: Synthesis of 2-[3-[(3S)-3-hydroxy-3-(5-methyl-1,3-oxazol-2-yl)but-1-yn-1-yl]phenyl]-7-methyl-7H-pyrrolo[2,3-d]pyrimidine-4-carboxamide

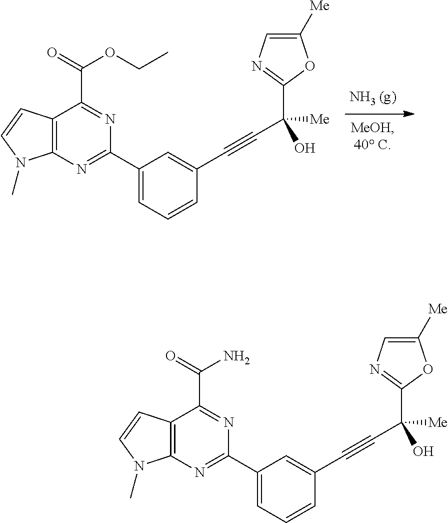 [SMILES: CCOC(=O)c1nc(-c2cccc(C#C[C@](C)(O)c3ncc(C)o3)c2)nc2c1ccn2C ; Cc1cnc([C@@](C)(O)C#Cc2cccc(-c3nc(C(N)=O)c4ccn(C)c4n3)c2)o1]

Similar to as described in General Procedure S, ethyl 2-[3-[(3S)-3-hydroxy-3-(5-methyl-1,3-oxazol-2-yl)but-1-yn-1-yl]phenyl]-7-methyl-7H-pyrrolo[2,3-d]pyrimidine-4-carboxylate was reacted with ammonia to afford the title compound (8.9 mg, 31%) as an off-white solid. LC-MS (ES, m/z): 402 [M+H]⁺. ¹H NMR (300 MHz, CD₃OD) δ 8.73 (s, 1H), 8.65-8.63 (m, 1H), 7.58-7.48 (m, 3H), 7.13 (d, J=3.6 Hz, 1H), 6.81 (d, J=1.2 Hz, 1H), 3.97 (s, 3H), 2.39 (s, 1H), 1.98 (s, 1H).

### Example AAA

### Synthesis of (S)-8-fluoro-2-(3-(3-hydroxy-3-(5-methyloxazol-2-yl)but-1-ynyl)phenyl)quinazoline-4-carboxamide

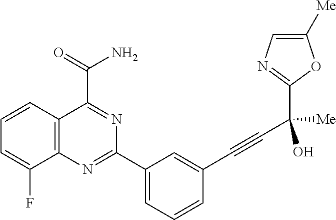 [SMILES: Cc1cnc([C@@](C)(O)C#Cc2cccc(-c3nc(C(N)=O)c4cccc(F)c4n3)c2)o1]

### Step 1: Synthesis of 2-chloro-4-(1-ethoxyethenyl)-8-fluoroquinazoline

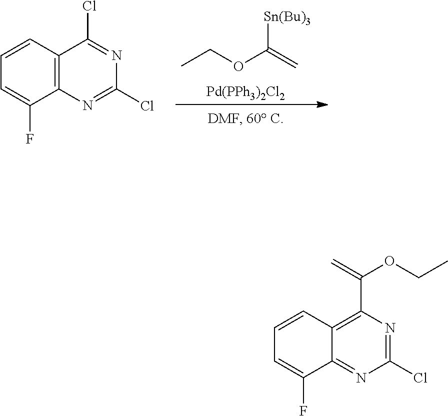

Similar to as described in General Procedure Q, 2,4-dichloro-8-fluoroquinazoline was reacted with tributyl(1-ethoxyethenyl)stannane to give the title compound (572 mg, 58%) as an off-white solid. LC-MS (ES, m/z): 253 [M+H]⁺.

### Step 2: Synthesis of ethyl 2-chloro-8-fluoroquinazoline-4-carboxylate

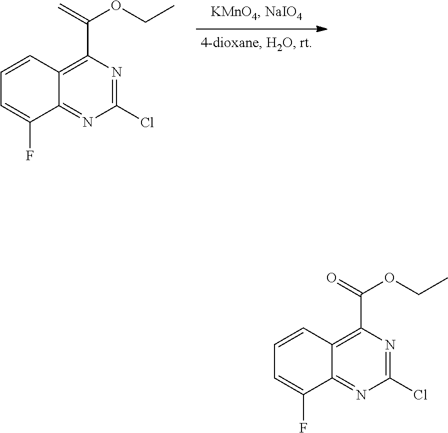 [SMILES: C=C(OCC)c1nc(Cl)nc2c(F)cccc12 ; CCOC(=O)c1nc(Cl)nc2c(F)cccc12]

Similar to as described in General Procedure R, 2-chloro-4-(1-ethoxyethenyl)-8-fluoroquinazoline was reacted with potassium permanganate(VII) to give the title compound (177 mg, 31%) as an off-white solid. LC-MS (ES, m/z): 255 [M+H]⁺.

### Step 3: Synthesis of ethyl 2-(3-bromophenyl)-8-fluoroquinazoline-4-carboxylate

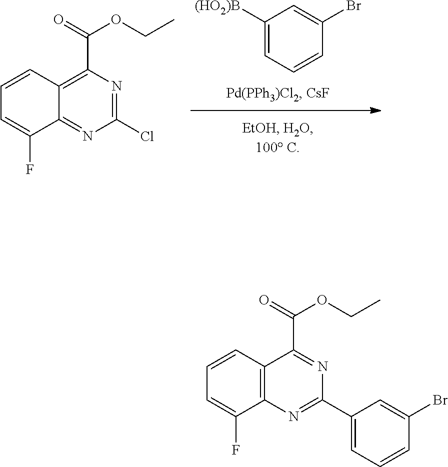

Similar to as described in General Procedure X, ethyl 2-chloro-8-fluoroquinazoline-4-carboxylate was reacted with (3-bromophenyl)boronic acid to give the title compound (77 mg, 48%) as an off-white solid. LC-MS (ES, m/z): 375 [M+H]⁺.

### Step 4: Synthesis of ethyl 8-fluoro-2-[3-[(3S)-3-hydroxy-3-(5-methyl-1,3-oxazol-2-yl)but-1-yn-1-yl]phenyl]quinazoline-4-carboxylate

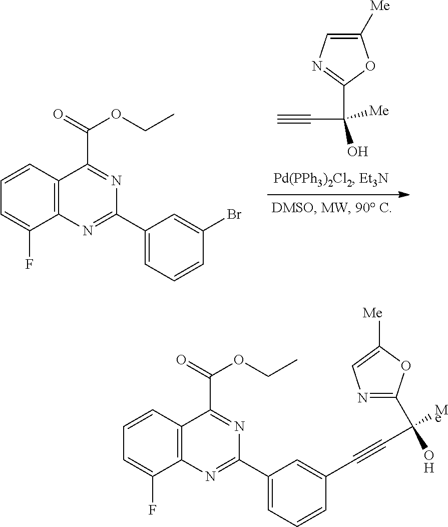 [SMILES: CCOC(=O)c1nc(-c2cccc(Br)c2)nc2c(F)cccc12 ; C#C[C@](C)(O)c1ncc(C)o1 ; CCOC(=O)c1nc(-c2cccc(C#C[C@](C)(O)c3ncc(C)o3)c2)nc2c(F)cccc12]

Similar to as described in General Procedure G, ethyl 2-(3-bromophenyl)-8-fluoroquinazoline-4-carboxylate was reacted with (2S)-2-(5-methyl-1,3-oxazol-2-yl)but-3-yn-2-ol to give the title compound (66 mg, 84%) as a light yellow solid. LC-MS (ES, m/z): 446 [M+H]⁺.

### Step 5: Synthesis of 8-fluoro-2-[3-[(3S)-3-hydroxy-3-(5-methyl-1,3-oxazol-2-yl)but-1-yn-1-yl]phenyl]quinazoline-4-carboxamide

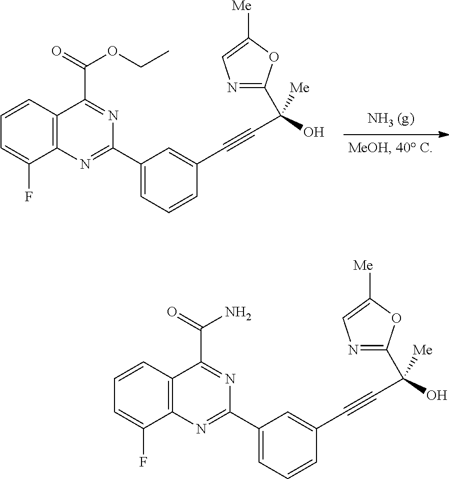 [SMILES: CCOC(=O)c1nc(-c2cccc(C#C[C@](C)(O)c3ncc(C)o3)c2)nc2c(F)cccc12 ; Cc1cnc([C@@](C)(O)C#Cc2cccc(-c3nc(C(N)=O)c4cccc(F)c4n3)c2)o1]

Similar to as described in General Procedure S, ethyl 8-fluoro-2-[3-[(3S)-3-hydroxy-3-(5-methyl-1,3-oxazol-2-yl)but-1-yn-1-yl]phenyl]quinazoline-4-carboxylate (66 mg, 0.15 mmol, 1.00 equiv) was reacted with ammonia to give the title compound (33.6 mg, 54%) as an off-white solid. LC-MS (ES, m/z): 417 [M+H]⁺. ¹H NMR (400 MHz, CD₃OD) δ 8.83 (s, 1H), 8.79-8.72 (m, 2H), 7.80-7.67 (m, 3H), 7.60-7.57 (t, J=7.6 Hz, 1H), 6.83 (s, 1H), 2.41-2.40 (s, 3H), 1.99 (s, 3H).

### Example BBB

### Synthesis of (S)-7-fluoro-2-(3-(3-hydroxy-3-(5-methyloxazol-2-yl)but-1-ynyl)phenyl)quinazoline-4-carboxamide

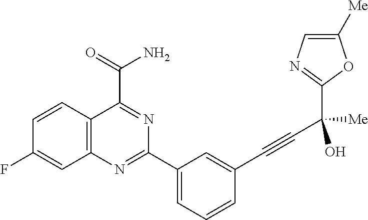 [SMILES: Cc1cnc([C@@](C)(O)C#Cc2cccc(-c3nc(C(N)=O)c4ccc(F)cc4n3)c2)o1]

### Step 1: Synthesis of ethyl 7-fluoro-2-[3-[(3S)-3-hydroxy-3-(5-methyl-1,3-oxazol-2-yl)but-1-yn-1-yl]phenyl]quinazoline-4-carboxylate

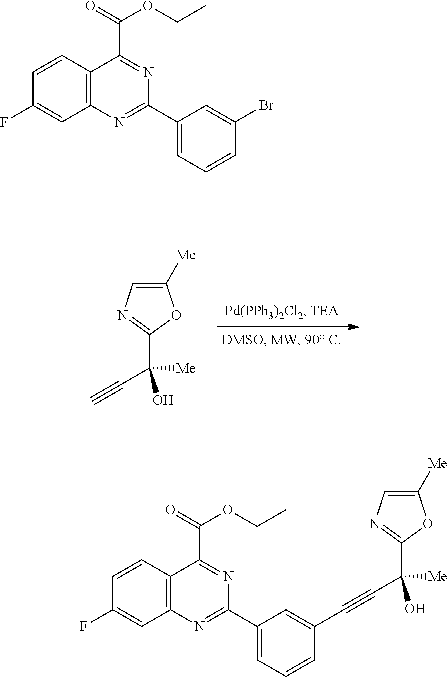 [SMILES: CCOC(=O)c1nc(-c2cccc(Br)c2)nc2cc(F)ccc12 ; C#C[C@](C)(O)c1ncc(C)o1 ; CCOC(=O)c1nc(-c2cccc(C#C[C@](C)(O)c3ncc(C)o3)c2)nc2cc(F)ccc12]

Similar to as described in General Procedure G, ethyl 2-(3-bromophenyl)-7-fluoroquinazoline-4-carboxylate was reacted with (2S)-2-(5-methyl-1,3-oxazol-2-yl)but-3-yn-2-ol to give the title compound (70 mg, 84%) as a dark red solid. LC-MS (ES, m/z): 446 [M+H]⁺.

### Step 2: Synthesis of 7-fluoro-2-[3-[(3S)-3-hydroxy-3-(5-methyl-1,3-oxazol-2-yl)but-1-yn-1-yl]phenyl]quinazoline-4-carboxamide

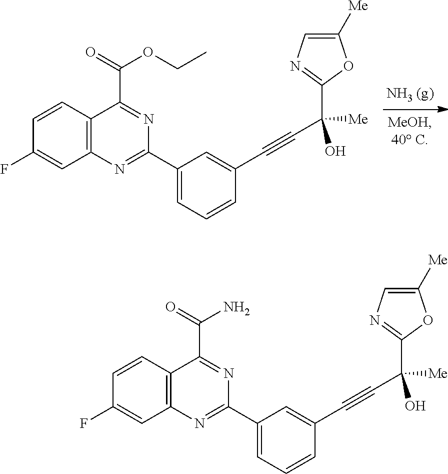 [SMILES: CCOC(=O)c1nc(-c2cccc(C#C[C@](C)(O)c3ncc(C)o3)c2)nc2cc(F)ccc12 ; Cc1cnc([C@@](C)(O)C#Cc2cccc(-c3nc(C(N)=O)c4ccc(F)cc4n3)c2)o1]

Similar to as described in General Procedure S, ethyl 7-fluoro-2-[3-[(3 S)-3-hydroxy-3-(5-methyl-1,3-oxazol-2-yl)but-1-yn-1-yl]phenyl]quinazoline-4-carboxylate was reacted with ammonia to give the title compound (36.5 mg, 56%) as an off-white solid. LC-MS (ES, m/z): 417 [M+H]⁺. ¹H NMR (300 MHz, DMSO-d₆) δ 8.95 (dd, J=9.3, 6.3 Hz, 1H), 8.72-8.68 (m, 3H), 8.18 (s, 1H), 7.96 (dd, J=12.6, 2.4 Hz, 1H), 7.77-7.62 (m, 3H), 6.84 (d, J=1.2 Hz, 1H), 6.71 (s, 3H), 2.34 (d, J=1.2 Hz, 3H), 1.89 (s, 3H).

### Example CCC

### Synthesis of (S)-ethyl 2-(3-(3-hydroxy-3-(5-methyloxazol-2-yl)but-1-ynyl)phenyl)-7-methoxyquinazoline-4-carboxylate

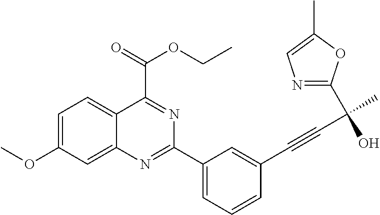 [SMILES: CCOC(=O)c1nc(-c2cccc(C#C[C@](C)(O)c3ncc(C)o3)c2)nc2cc(OC)ccc12]

### Step 1: Synthesis of ethyl 2-(3-bromophenyl)-7-methoxyquinazoline-4-carboxylate

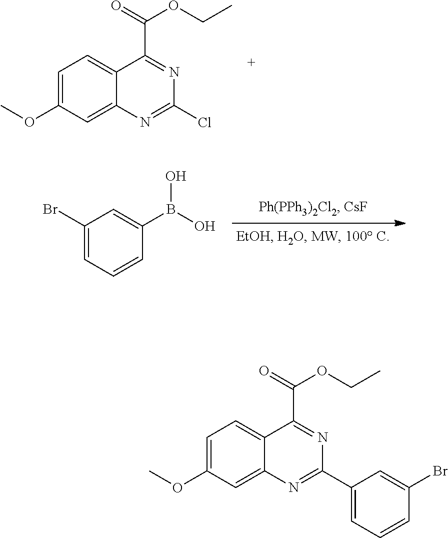 [SMILES: CCOC(=O)c1nc(Cl)nc2cc(OC)ccc12 ; OB(O)c1cccc(Br)c1 ; CCOC(=O)c1nc(-c2cccc(Br)c2)nc2cc(OC)ccc12]

Similar to as described in General Procedure X, ethyl 2-chloro-7-methoxyquinazoline-4-carboxylate was reacted with (3-bromophenyl)boronic acid to give the title compound (90.0 mg, 52%) as an off-white solid. LC-MS (ES, m/z): 387 [M+H]⁺.

### Step 2: Synthesis of ethyl 2-[3-[(3S)-3-hydroxy-3-(5-methyl-1,3-oxazol-2-yl)but-1-yn-1-yl]phenyl]-7-methoxyquinazoline-4-carboxylate

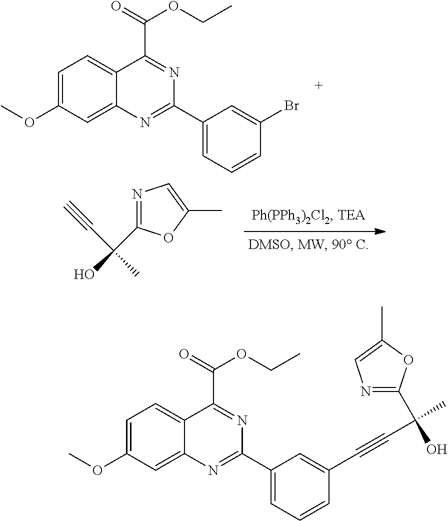

Similar to as described in General Procedure G, ethyl 2-(3-bromophenyl)-7-methoxyquinazoline-4-carboxylate was reacted with (2S)-2-(5-methyl-1,3-oxazol-2-yl)but-3-yn-2-ol to give the title compound (90.0 mg, 85%) as a light yellow solid. LC-MS (ES, m/z): 458 [M+H]⁺.

### Step 3: Synthesis of 2-[3-[(3S)-3-hydroxy-3-(5-methyl-1,3-oxazol-2-yl)but-1-yn-1-yl]phenyl]-7-methoxyquinazoline-4-carboxamide

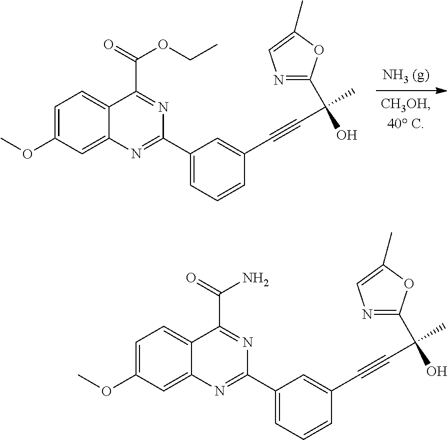

Similar to as described in General Procedure S, ethyl 2-[3-[(3S)-3-hydroxy-3-(5-methyl-1,3-oxazol-2-yl)but-1-yn-1-yl]phenyl]-7-methoxyquinazoline-4-carboxylate was reacted with ammonia to give the title compound (33.7 mg, 40%) as an off-white solid. LC-MS (ES, m/z): 429 [M+H]⁺. ¹H NMR (300 MHz, CD₃OD) δ 8.75 (d, J=9.3 Hz, 1H), 8.69-8.67 (m, 2H), 8.63 (s, 1H), 8.07 (s, 1H), 7.63-7.56 (m, 2H), 7.52 (d, J=2.4 Hz, 1H), 7.39 (dd, J=9.3, 2.7 Hz, 1H), 6.83 (d, J=1.2 Hz, 1H), 6.69 (s, 1H), 4.01 (s, 3H), 2.33 (d, J=1.2 Hz, 3H), 1.88 (s, 3H).

### Example DDD

### Synthesis of (S)-2-(3-(3-hydroxy-3-(5-methyloxazol-2-yl)but-1-ynyl)phenyl)-8-methoxyquinazoline-4-carboxamide

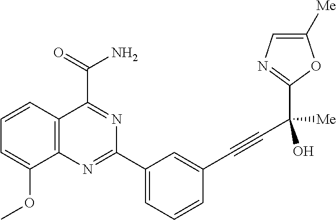 [SMILES: COc1cccc2c(C(N)=O)nc(-c3cccc(C#C[C@](C)(O)c4ncc(C)o4)c3)nc12]

### Step 1: Synthesis of ethyl 2-[3-[(3S)-3-hydroxy-3-(5-methyl-1,3-oxazol-2-yl)but-1-yn-1-yl]phenyl]-8-methoxyquinazoline-4-carboxylate

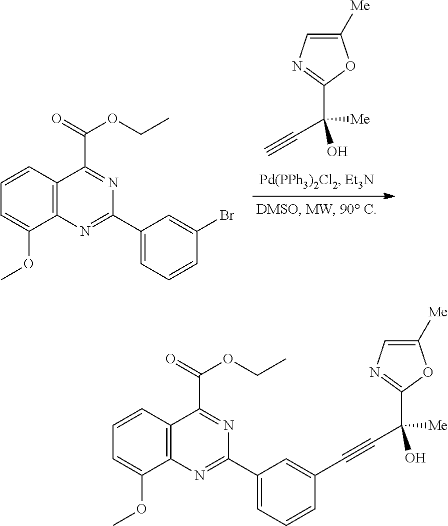 [SMILES: CCOC(=O)c1nc(-c2cccc(Br)c2)nc2c(OC)cccc12 ; C#C[C@](C)(O)c1ncc(C)o1 ; CCOC(=O)c1nc(-c2cccc(C#C[C@](C)(O)c3ncc(C)o3)c2)nc2c(OC)cccc12]

Similar to as described in General Procedure G, ethyl 2-(3-bromophenyl)-8-methoxyquinazoline-4-carboxylate was reacted with (2S)-2-(5-methyl-1,3-oxazol-2-yl)but-3-yn-2-ol to give the title compound (100 mg, 71%) as a yellow solid. LC-MS (ES, m/z): 458 [M+H]⁺.

### Step 2: Synthesis of 2-[3-[(3S)-3-hydroxy-3-(5-methyl-1,3-oxazol-2-yl)but-1-yn-1-yl]phenyl]-8-methoxyquinazoline-4-carboxamide

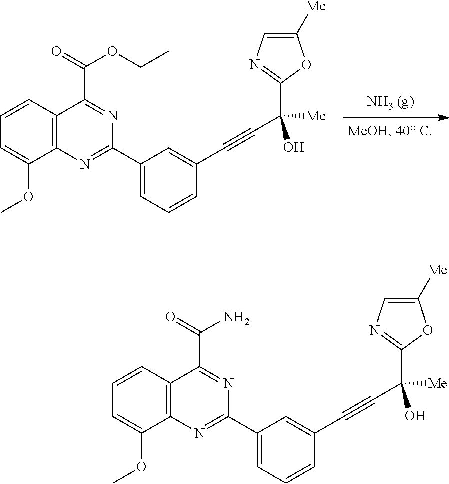 [SMILES: CCOC(=O)c1nc(-c2cccc(C#C[C@](C)(O)c3ncc(C)o3)c2)nc2c(OC)cccc12 ; COc1cccc2c(C(N)=O)nc(-c3cccc(C#C[C@](C)(O)c4ncc(C)o4)c3)nc12]

Similar to as described in General Procedure S, ethyl 2-[3-[(3S)-3-hydroxy-3-(5-methyl-1,3-oxazol-2-yl)but-1-yn-1-yl]phenyl]-8-methoxyquinazoline-4-carboxylate was reacted with ammonia to give the title compound (24.1 mg, 26%) as a light yellow solid. LC-MS (ES, m/z) 429 [M+H]⁺. ¹H NMR (400 MHz, CD₃OD) δ 8.78 (s, 1H), 8.68 (d, J=8.0 Hz, 1H), 8.43 (d, J=8.4 Hz, 1H), 7.69-7.63 (m, 2H), 7.58-7.54 (m, 1H), 7.48 (d, J=7.6 Hz, 1H), 6.83 (d, J=1.2 Hz, 1H), 4.14 (s, 1H), 2.41 (s, 3H), 1.99 (s, 3H).

### Example EEE

### Synthesis of (R)-3-amino-5-fluoro-6-(3-((3-hydroxy-1-methyl-2-oxopyrrolidin-3-yl)ethynyl)phenyl)picolinamide

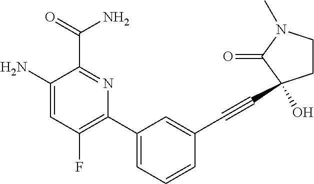 [SMILES: CN1CC[C@@](O)(C#Cc2cccc(-c3nc(C(N)=O)c(N)cc3F)c2)C1=O]

### Step 1: Synthesis of methyl 3-amino-5-fluoro-6-(3-[2-[(3R)-3-hydroxy-1-methyl-2-oxopyrrolidin-3-yl]ethynyl]phenyl)pyridine-2-carboxylate

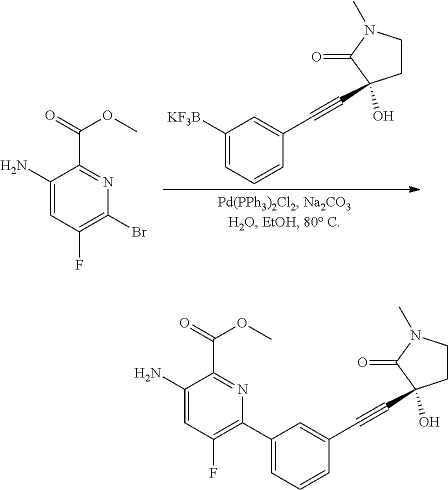

Similar to as described in General Procedure U, methyl 3-amino-6-bromo-5-fluoropyridine-2-carboxylate was reacted with potassium trifluoro(3-2-[(3R)-3-hydroxy-1-methyl-2-oxopyrrolidin-3-yl]ethynylphenyl)boranideto give the title compound (300 mg, 39%) as a light yellow solid. LC-MS (ES, m/z): 384 [M+H]⁺.

### Step 2: Synthesis of 3-amino-5-fluoro-6-(3-[2-[(3R)-3-hydroxy-1-methyl-2-oxopyrrolidin-3-yl]ethynyl]phenyl)pyridine-2-carboxamide

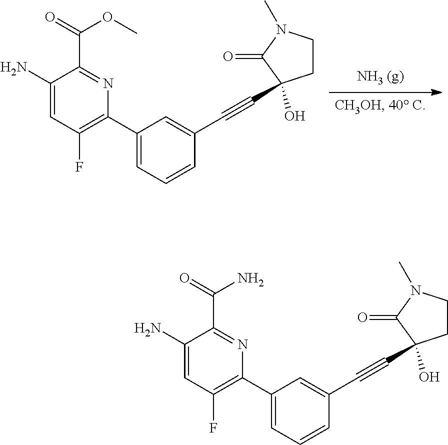 [SMILES: COC(=O)c1nc(-c2cccc(C#C[C@]3(O)CCN(C)C3=O)c2)c(F)cc1N ; CN1CC[C@@](O)(C#Cc2cccc(-c3nc(C(N)=O)c(N)cc3F)c2)C1=O]

Similar to as described in General Procedure S, methyl 3-amino-5-fluoro-6-(3-[2-[(3R)-3-hydroxy-1-methyl-2-oxopyrrolidin-3-yl]ethynyl]phenyl)pyridine-2-carboxylate was reacted with ammonia to give the title compound (1.04 g, 54%) as a white solid. LC-MS (ES, m/z): 369 [M+H]⁺. ¹H NMR (400 MHz, CD₃OD) δ 7.99 (s, 1H), 7.92 (m, 2H), 7.48-7.42 (m, 2H), 6.99 (d, J=13.6 Hz, 1H), 3.54-3.46 (m, 2H), 2.95 (s, 3H), 2.64-2.58 (m, 1H), 2.37-2.30 (m, 1H).

### Example FFF

### Synthesis of (R)-3-chloro-5-fluoro-6-(3-((3-hydroxy-1-methyl-2-oxopyrrolidin-3-yl)ethynyl)phenyl)picolinamide

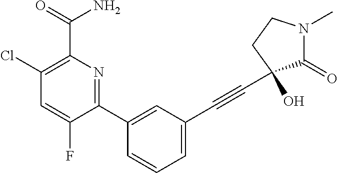 [SMILES: CN1CC[C@@](O)(C#Cc2cccc(-c3nc(C(N)=O)c(Cl)cc3F)c2)C1=O]

### Step 1: Synthesis of methyl 3-amino-5-fluoropyridine-2-carboxylate

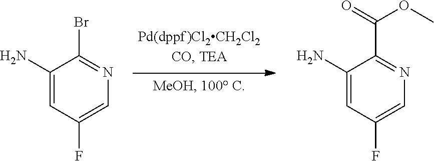

Similar to as described in General Procedure 0, 2-bromo-5-fluoropyridin-3-amine was reacted with carbon monoxide to give the title compound (150 mg, 4%) as a light yellow solid. LC-MS (ES, m/z): 171 [M+H]⁺.

### Step 2: Synthesis of methyl 3-amino-6-bromo-5-fluoropyridine-2-carboxylate

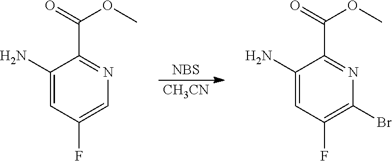

A solution of methyl 3-amino-5-fluoropyridine-2-carboxylate (150.00 mg, 0.88 mmol, 1.00 equiv), NBS (172.61 mg, 0.97 mmol, 1.10 equiv) in acetonitrile (15 mL) was stirred for 3 h at room temperature. The resulting mixture was concentrated under vacuum and the residue was applied onto a silica gel column with ethyl acetate/petroleum ether (1:3) to give the title compound (200 mg, 91%) as a light yellow solid. LC-MS (ES, m/z): 249.251 [M+H]⁺.

### Step 3: Synthesis of methyl 6-bromo-3-chloro-5-fluoropyridine-2-carboxylate

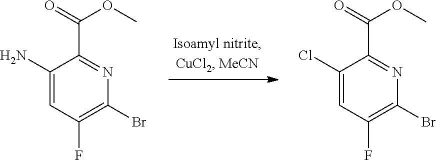

A mixture of methyl 3-amino-6-bromo-5-fluoropyridine-2-carboxylate (150.00 mg, 0.60 mmol, 1.00 equiv), 3-methylbutyl nitrite (141.12 mg, 1.20 mmol, 2.00 equiv), acetonitrile (5 mL), and copper(II)chloride (97.18 mg, 0.72 mmol, 1.20 equiv) in a sealed tube under nitrogen was stirred for 24 h at 60° C. The resulting mixture was concentrated under vacuum and the residue was applied onto a silica gel column with ethyl acetate/petroleum ether (1:3) to give the title compound (120 mg, 74%) as a light yellow solid. LC-MS (ES, m/z): 268 [M+H]⁺.

### Step 4: Synthesis of methyl 3-chloro-5-fluoro-6-(3-[2-[(3R)-3-hydroxy-1-methyl-2-oxopyrrolidin-3-yl]ethynyl]phenyl)pyridine-2-carboxylate

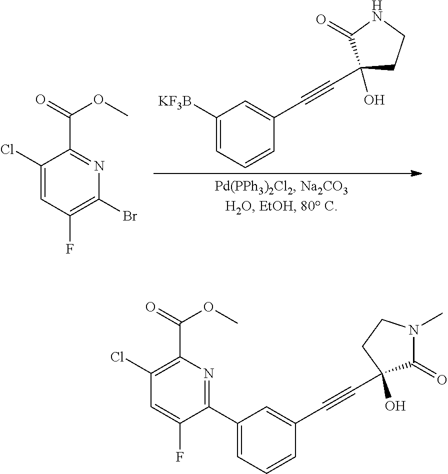

Similar to as described in General Procedure U, methyl 6-bromo-3-chloro-5-fluoropyridine-2-carboxylate was reacted with potassium trifluoro(3-2-[(3R)-3-hydroxy-1-methyl-2-oxopyrrolidin-3-yl]ethynylphenyl)boranideto give the title compound (76 mg, 46%) as light yellow oil. LC-MS (ES, m/z): 403 [M+H]⁺.

### Step 5: Synthesis of 3-chloro-5-fluoro-6-(3-[2-[(3R)-3-hydroxy-1-methyl-2-oxopyrrolidin-3-yl]ethynyl]phenyl)pyridine-2-carboxamide

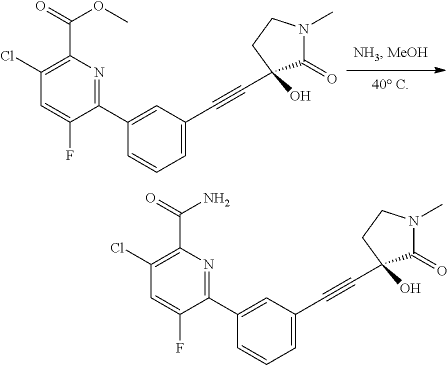

Similar to as described in General Procedure S, methyl 3-chloro-5-fluoro-6-(3-[2-[(3R)-3-hydroxy-1-methyl-2-oxopyrrolidin-3-yl]ethynyl]phenyl)pyridine-2-carboxylate was reacted with ammonia to give the title compound (23 mg, 36%) as a white solid. LC-MS (ES, m/z): 388 [M+H]⁺. ¹H NMR (300 MHz, CD₃OD) δ 8.14-8.13 (d, J=1.2 Hz, 1H), 8.06-7.99 (m, 2H), 7.62-7.51 (m, 2H), 3.53-3.48 (m, 2H), 2.95 (s, 3H), 2.66-2.58 (m, 1H), 2.38-2.29 (m, 1H).

### Example GGG

### Synthesis of (S)-5-(3-((3-hydroxy-1-methyl-2-oxopyrrolidin-3-yl)ethynyl)phenyl)imidazo[1,2-f]pyrimidine-7-carboxamide

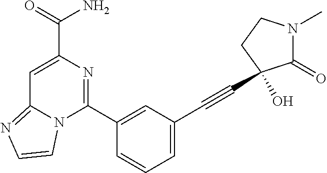 [SMILES: CN1CC[C@](O)(C#Cc2cccc(-c3nc(C(N)=O)cc4nccn34)c2)C1=O]

### Step 1: Synthesis of methyl 6-amino-2-chloropyrimidine-4-carboxylate

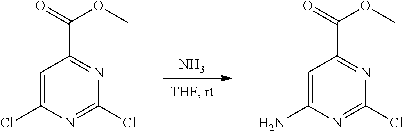

Similar to as described in General Procedure A, 2,6-dichloropyrimidine-4-carboxylate was reacted with ammonia to give the title compound (240 mg, 44%) as a light yellow solid. LC-MS (ES, m/z): 188 [M+H]⁺.

### Step 2: Synthesis of methyl 5-chloroimidazo[1,2-c]pyrimidine-7-carboxylate

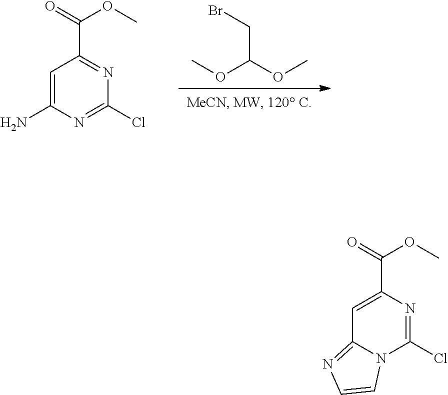

A mixture of methyl 6-amino-2-chloropyrimidine-4-carboxylate (260 mg, 1.39 mmol, 1.00 equiv) and 2-bromo-1,1-dimethoxyethane (1.1 g, 6.51 mmol, 4.00 equiv) in acetonitrile (6 mL) was irradiated with microwave radiation for 1 h at 120° C. The solids were collected by filtration to give the title compound (162 mg, 55%) as an off-white solid. LC-MS (ES, m/z): 212 [M+H]⁺.

### Step 3: Synthesis of ethyl 5-(3-[2-[(3R)-3-hydroxy-1-methyl-2-oxopyrrolidin-3-yl]ethynyl]phenyl)imidazo[1,2-c]pyrimidine-7-carboxylate

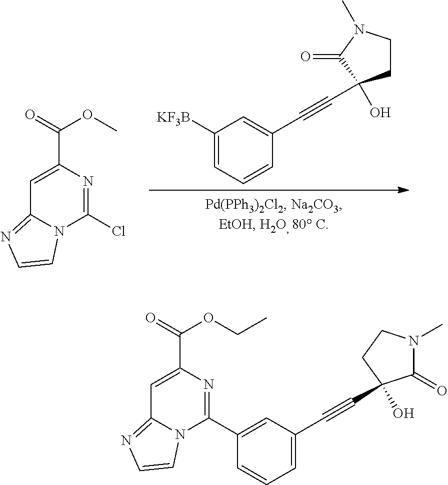

Similar to as described in General Procedure U, methyl 5-chloroimidazo[1,2-c]pyrimidine-7-carboxylate was reacted with potassium trifluoro(3-2-[(3R)-3-hydroxy-1-methyl-2-oxopyrrolidin-3-yl]ethynylphenyl)boranideto give the title compound (80 mg, 42%) as an off-white solid. LC-MS (ES, m/z): 405 [M+H]⁺.

### Step 4: Synthesis of 5-(3-[2-[(3R)-3-hydroxy-1-methyl-2-oxopyrrolidin-3-yl]ethynyl]phenyl)imidazo[1,2-c]pyrimidine-7-carboxamide

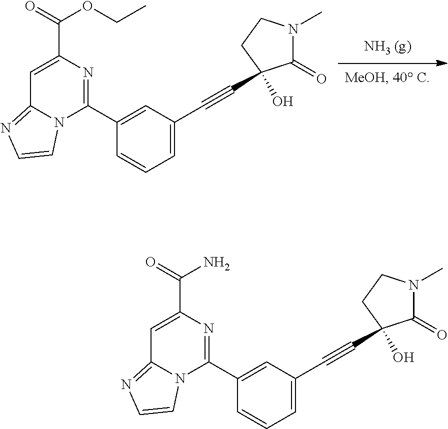 [SMILES: CCOC(=O)c1cc2nccn2c(-c2cccc(C#C[C@@]3(O)CCN(C)C3=O)c2)n1 ; CN1CC[C@](O)(C#Cc2cccc(-c3nc(C(N)=O)cc4nccn34)c2)C1=O]

Similar to as described in General Procedure S, ethyl 5-(3-[2-[(3R)-3-hydroxy-1-methyl-2-oxopyrrolidin-3-yl]ethynyl]phenyl)imidazo[1,2-c]pyrimidine-7-carboxylate was reacted with ammonia to give the title compound (19.5 mg, 26%) as a white solid. LC-MS (ES, m/z): 376 [M+H]⁺. ¹H NMR (400 MHz, CD₃OD) δ 8.29 (s, 1H), 8.16 (s, 2H),8.06 (d, J=7.6 Hz, 1H), 7.85 (s, 1H), 7.78 (d, J=8.0 Hz, 1H), 7.69-7.66 (t, J=8.0 Hz, 1H), 3.51-3.48 (m, 2H), 2.94 (s, 3H), 2.64-2.58 (m, 1H), 2.37-2.30 (m, 1H).

### Example HHH

### Synthesis of (R)-3-amino-6-(3-((3-hydroxy-1-methyl-2-oxopyrrolidin-3-yl)ethynyl)phenyl)pyrazine-2-carboxamide

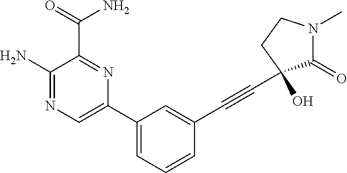 [SMILES: CN1CC[C@@](O)(C#Cc2cccc(-c3cnc(N)c(C(N)=O)n3)c2)C1=O]

### Step 1: Synthesis of methyl 3-amino-6-(3-bromophenyl)pyrazine-2-carboxylate

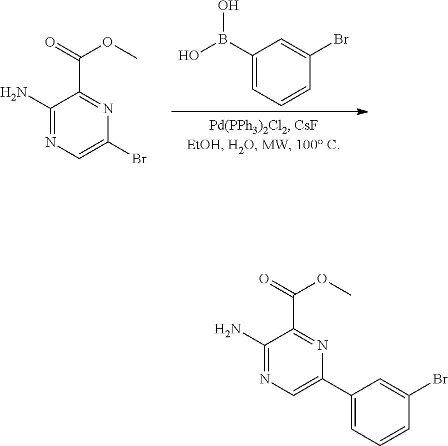 [SMILES: COC(=O)c1nc(Br)cnc1N ; OB(O)c1cccc(Br)c1 ; COC(=O)c1nc(-c2cccc(Br)c2)cnc1N]

Similar to as described in General Procedure X, methyl 3-amino-6-bromopyrazine-2-carboxylate was reacted with (3-bromophenyl)boronic acid (201 mg, 1.00 mmol, 1.00 equiv) to give the title compound (170 mg, 55%) as a yellow solid. LC-MS (ES, m/z): 308 [M+H]⁺.

### Step 2: Synthesis of 3-amino-6-(3-bromophenyl)pyrazine-2-carboxamide

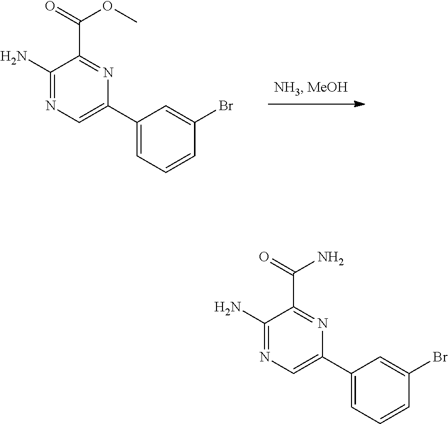 [SMILES: COC(=O)c1nc(-c2cccc(Br)c2)cnc1N ; NC(=O)c1nc(-c2cccc(Br)c2)cnc1N]

Similar to as described in General Procedure S, methyl 3-amino-6-(3-bromophenyl)pyrazine-2-carboxylate was reacted with ammonia to give the title compound (130 mg, 80%) as a yellow solid. LC-MS (ES, m/z): 293 [M+H]⁺.

### Step 3: Synthesis of 3-amino-6-(3-[2-[(3R)-3-hydroxy-1-methyl-2-oxopyrrolidin-3-yl]ethynyl]phenyl)pyrazine-2-carboxamide

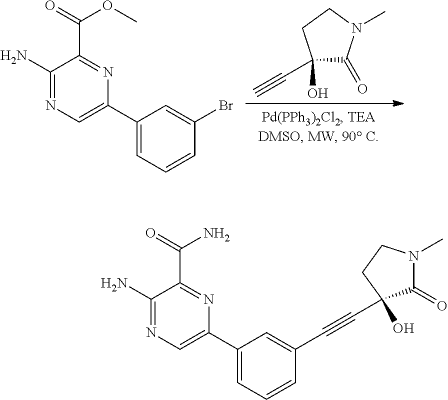

Similar to as described in General Procedure G, 3-amino-6-(3-bromophenyl)pyrazine-2-carboxamide (120 mg, 0.41 mmol, 1.00 equiv) was reacted with (3R)-3-ethynyl-3-hydroxy-1-methylpyrrolidin-2-one to give the title compound (20.7 mg, 14.4%) as a yellow solid. LC-MS (ES, m/z): 352 [M+H]⁺. ¹H NMR (400 MHz, CD₃OD) δ 8.73 (s, 1H), 8.15 (s, 1H), 8.06-8.03 (m, 1H), 7.47 (d, J=4.8 Hz, 2H), 3.55-3.47 (m, 2H), 2.95 (s, 3H), 2.65-2.59 (m, 1H), 2.37-2.30 (m, 1H).

### Example III

### Synthesis of (R)-3-amino-6-(3-((3-hydroxy-1-methyl-2-oxopyrrolidin-3-yl)ethynyl)phenyl)picolinamide

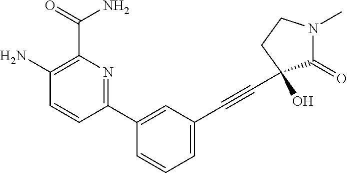 [SMILES: CN1CC[C@@](O)(C#Cc2cccc(-c3ccc(N)c(C(N)=O)n3)c2)C1=O]

### Step 1: Synthesis of methyl 3-amino-6-bromopyridine-2-carboxylate

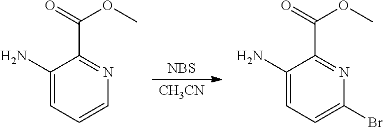

A solution of methyl 3-aminopyridine-2-carboxylate (1 g, 6.57 mmol, 1.00 equiv) and N-bromosuccinimide (1.29 g, 7.25 mmol, 1.10 equiv) in acetonitrile (25 mL) was stirred at room temperature. After being stirred for 2 h the mixture was concentrated under vacuum. The residue was applied onto a silica gel column with ethyl acetate/petroleum ether (2:1) to give the title compound (0.81 g, 54%) as a yellow solid. LC-MS (ES, m/z): 231, 233 [M+H]⁺.

### Step 2: Synthesis of 3-amino-6-bromopyridine-2-carboxamide

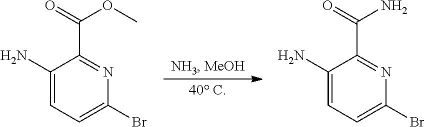

Similar to as described in General Procedure S, methyl 3-amino-6-bromopyridine-2-carboxylate was reacted with ammonia to give the title compound (780 mg, 71%) as a yellow solid. LC-MS (ES, m/z): 216, 218 [M+H]⁺.

### Step 3: Synthesis of 3-amino-6-(3-[2-[(3R)-3-hydroxy-1-methyl-2-oxopyrrolidin-3-yl]ethynyl]phenyl)pyridine-2-carboxamide

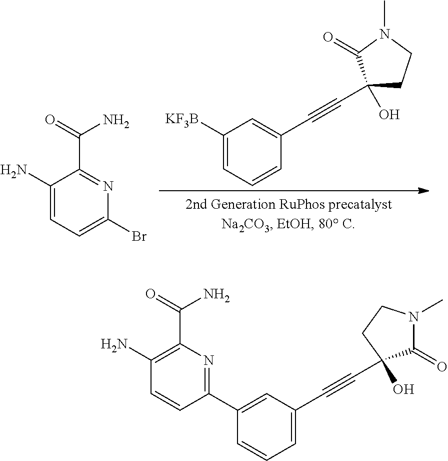

Similar to as described in General Procedure U, 3-amino-6-bromopyridine-2-carboxamide3-amino-6-bromopyridine-2-carboxamide was reacted with potassium trifluoro(3-2-[(3R)-3-hydroxy-1-methyl-2-oxopyrrolidin-3-yl]ethynylphenyl)boranideto give the title compound (45.1 mg, 28%) as a white solid. LC-MS (ES, m/z): 351 [M+H]⁺. ¹H NMR (300 MHz, CD₃OD) δ 8.11 (s, 1H), 8.03-7.99 (m, 1H), 7.80 (d, J=8.7 Hz, 1H), 7.46-7.42 (m, 2H), 7.27 (d, J=8.7 Hz, 1H), 3.53-3.48 (m, 2H), 2.95 (s, 3H), 2.66-2.58 (m, 1H), 2.38-2.29 (m, 1H).

### Example JJJ and Example KKK

### Synthesis of (R)-3-amino-6-(3-((3-hydroxy-2-oxo-tetrahydrofuran-3-yl)ethynyl)phenyl)picolinamide and (S)-3-amino-6-(3-((3-hydroxy-2-oxo-tetrahydrofuran-3-yl)ethynyl)phenyl)picolinamide

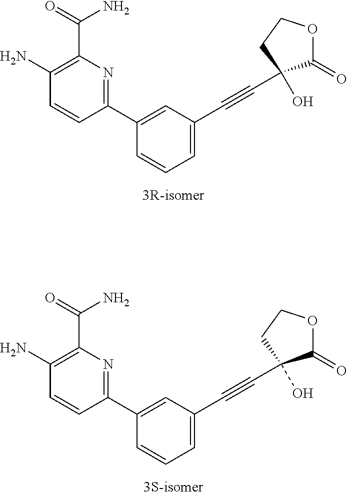 [SMILES: NC(=O)c1nc(-c2cccc(C#C[C@]3(O)CCOC3=O)c2)ccc1N ; NC(=O)c1nc(-c2cccc(C#C[C@@]3(O)CCOC3=O)c2)ccc1N]

### Step 1: Synthesis of 3-amino-6-(3-bromophenyl)picolinamide

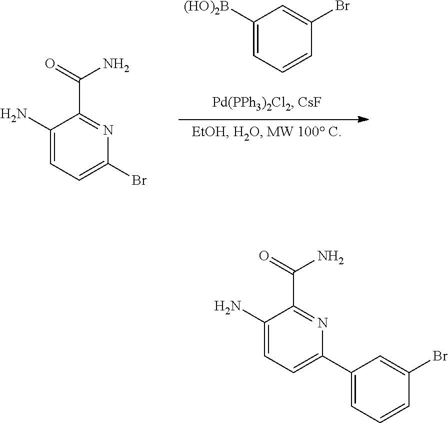 [SMILES: NC(=O)c1nc(Br)ccc1N ; OB(O)c1cccc(Br)c1 ; NC(=O)c1nc(-c2cccc(Br)c2)ccc1N]

Similar to as described in General Procedure X, 3-amino-6-bromopicolinamide was reacted with 3-bromo-phenyl boronic acid to give the title compound (62 mg, crude) as a white solid. LC-MS (ES, m/z): 292, 294 [M+H]⁺.

### Step 2: Synthesis of (R)-3-amino-6-(3-((3-hydroxy-2-oxo-tetrahydrofuran-3-yl)ethynyl)phenyl)picolinamide and (S)-3-amino-6-(3-((3-hydroxy-2-oxo-tetrahydrofuran-3-yl)ethynyl)phenyl)picolinamide

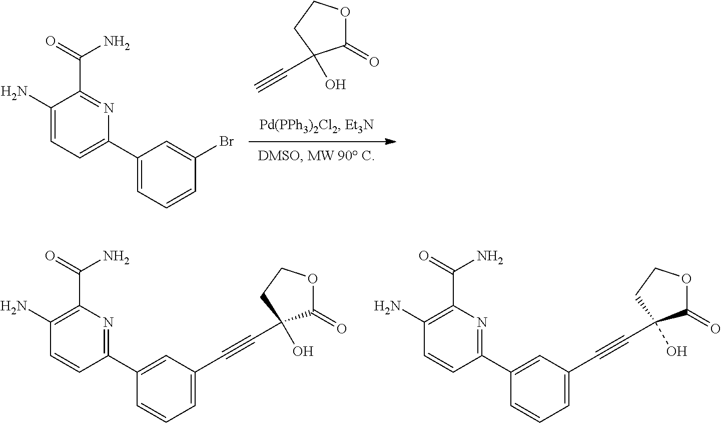

Similar to as described in General Procedure G, 3-amino-6-(3-bromophenyl)picolinamide was reacted with 3-ethynyl-3-hydroxyoxolan-2-one to give 20 mg (12%) of 3-amino-6-(3-[2-[(3R)-3-hydroxy-2-oxooxolan-3-yl]ethynyl]phenyl)pyridine-2-carboxamide as a yellow solid and resulted in 20 mg (12%) of 3-amino-6-(3-[2-[(3 S)-3-hydroxy-2-oxooxolan-3-yl]ethynyl]phenyl)pyridine-2-carboxamide as a yellow solid.

The stereochemistry of both isomers was arbitrarily assigned. Isomer A (3R-isomer): t_{R}=17.14 min (CHIRALPAK IC-3, 0.46*15 cm, Hex:EtOH=70:30, 1.0 ml/min); Isomer B (3S-isomer): t_{R}=14.67 min CHIRALPAK IC-3, 0.46*15 cm, (Hex:EtOH=70:30, 1.0 ml/min); Both isomers showed identical LC-MS and ¹H NMR as shown below.

LC-MS (ES, m/z): 338 [M+H]⁺. ¹H NMR (CD₃OD): 8.13 (s, 1H), 8.07-8.04 (m, 1H), 7.82 (d, J=8.7 Hz, 2H), 7.47 (d, J=5.1 Hz, 2H), 7.28 (d, J=8.7 Hz, 1H), 4.50-4.47 (m, 2H), 2.80-2.75 (m, 1H), 2.63-2.54 (m, 1H).

### Example LLL

### Synthesis of (R)-2-(3-((3-hydroxy-1-methyl-2-oxopyrrolidin-3-yl)ethynyl)phenyl)-6-(1-methyl-1H-imidazol-5-yl)pyrimidine-4-carboxamide

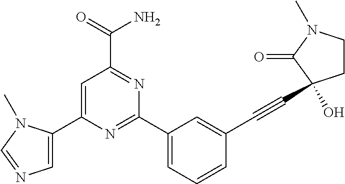 [SMILES: CN1CC[C@@](O)(C#Cc2cccc(-c3nc(C(N)=O)cc(-c4cncn4C)n3)c2)C1=O]

### Step 1: Synthesis of (R)-ethyl 2-(3-((3-hydroxy-1-methyl-2-oxopyrrolidin-3-yl)ethynyl)phenyl)-6-(1-methyl-1H-imidazol-5-yl)pyrimidine-4-carboxylate

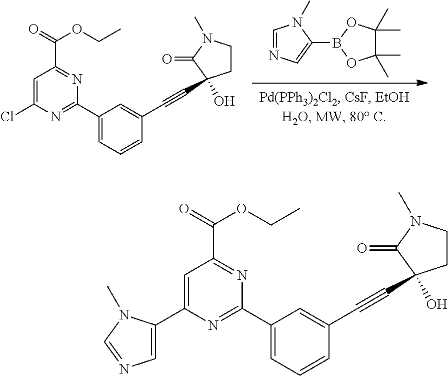 [SMILES: CCOC(=O)c1cc(Cl)nc(-c2cccc(C#C[C@]3(O)CCN(C)C3=O)c2)n1 ; Cn1cncc1B1OC(C)(C)C(C)(C)O1 ; CCOC(=O)c1cc(-c2cncn2C)nc(-c2cccc(C#C[C@]3(O)CCN(C)C3=O)c2)n1]

Similar to as described in General Procedure X, ethyl 6-chloro-2-(3-[2-[(3R)-3-hydroxy-1-methyl-2-oxopyrrolidin-3-yl]ethynyl]phenyl)pyrimidine-4-carboxylate was reacted with 1-methyl-5-(tetramethyl-1,3,2-dioxaborolan-2-yl)-1H-imidazole to give the title compound (133 mg, 49%) as a light yellow solid. LC-MS (ES, m/z): 446 [M+H]⁺.

### Step 2: Synthesis of 2-(3-[2-[(3R)-3-hydroxy-1-methyl-2-oxopyrrolidin-3-yl]ethynyl]phenyl)-6-(1-methyl-1H-imidazol-5-yl)pyrimidine-4-carboxamide

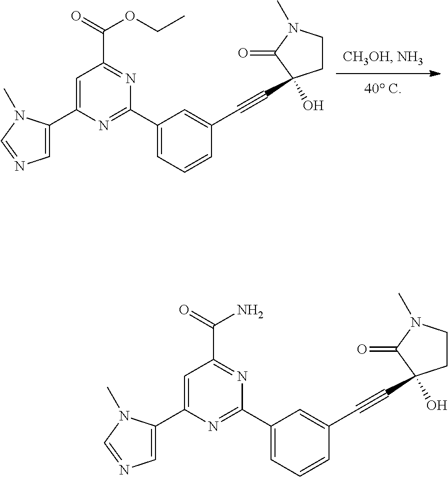 [SMILES: CCOC(=O)c1cc(-c2cncn2C)nc(-c2cccc(C#C[C@]3(O)CCN(C)C3=O)c2)n1 ; CN1CC[C@@](O)(C#Cc2cccc(-c3nc(C(N)=O)cc(-c4cncn4C)n3)c2)C1=O]

Similar to as described in General Procedure S, ethyl 2-(3-[2-[(3R)-3-hydroxy-1-methyl-2-oxopyrrolidin-3-yl]ethynyl]phenyl)-6-(1-methyl-1H-imidazol-5-yl)pyrimidine-4-carboxylate was reacted with ammonia to give the title compound (43.9 mg, 35%) as a light yellow solid. LC-MS (ES, m/z): 417 [M+H]⁺. ¹H NMR (CD₃OD, 400 MHz) δ 8.70 (s, 1H), 8.61 (d, J=8.0 Hz, 1H), 8.28 (s, 1H), 7.98 (s, 1H), 7.94 (s, 1H), 7.66 (d, J=7.6 Hz, 1H), 7.58-7.54 (t, J=7.8 Hz, 1H), 4.29 (s, 3H), 3.53-3.43 (m, 2H), 2.96 (s, 3H), 2.66-2.60 (m, 1H), 2.39-2.32 (m, 1H).

### Example MMM

### Synthesis of (R)-6-(1-ethyl-1H-pyrazol-5-yl)-2-(3-((3-hydroxy-1-methyl-2-oxopyrrolidin-3-yl)ethynyl)phenyl)pyrimidine-4-carboxamide

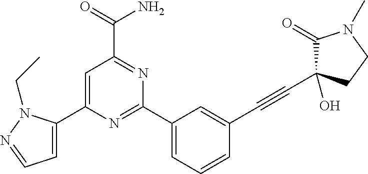 [SMILES: CCn1nccc1-c1cc(C(N)=O)nc(-c2cccc(C#C[C@]3(O)CCN(C)C3=O)c2)n1]

### Step 1: Synthesis of ethyl 2-chloro-6-(1-ethyl-1H-pyrazol-5-yl)pyrimidine-4-carboxylate

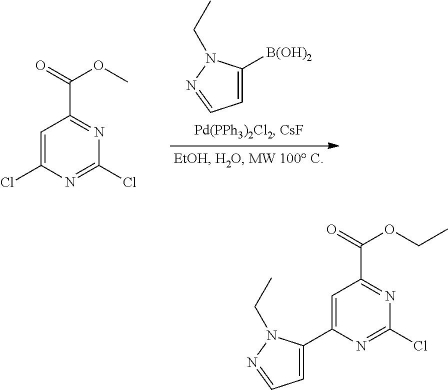

Similar to as described in General Procedure X, methyl 2,6-dichloropyrimidine-4-carboxylate was reacted with (1-ethyl-1H-pyrazol-5-yl)boronic acid to give the title compound (108 mg, 40%) as an off-white solid. LC-MS (ES, m/z): 281 [M+H]⁺.

### Step 2: Synthesis of 6-(1-ethyl-1H-pyrazol-5-yl)-2-(3-[2-[(3R)-3-hydroxy-1-methyl-2-oxopyrrolidin-3-yl]ethynyl]phenyl)pyrimidine-4-carboxylic acid

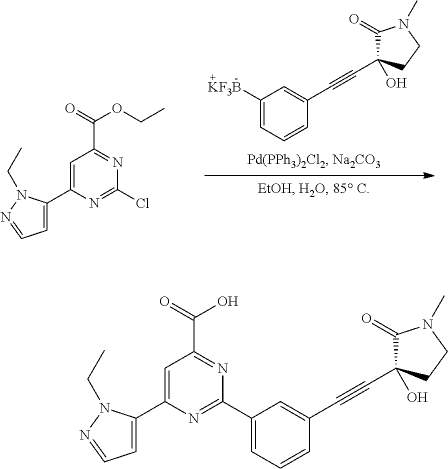

Similar to as described in General Procedure U, ethyl 2-chloro-6-(1-ethyl-1H-pyrazol-5-yl)pyrimidine-4-carboxylate was reacted with potassium trifluoro(3-2-[(3R)-3-hydroxy-1-methyl-2-oxopyrrolidin-3-yl]ethynylphenyl)boranideto give the title compound (36 mg, 12%) as a brown solid. LC-MS (ES, m/z): 432 [M+H]⁺.

### Step 3: Synthesis of 6-(1-ethyl-1H-pyrazol-5-yl)-2-(3-[2-[(3R)-3-hydroxy-1-methyl-2-oxopyrrolidin-3-yl]ethynyl]phenyl)pyrimidine-4-carboxamide

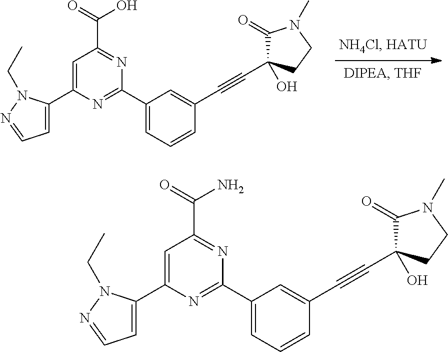

Similar to as described in General Procedure B, 6-(1-ethyl-1H-pyrazol-5-yl)-2-(3-[2-[(3R)-3-hydroxy-1-methyl-2-oxopyrrolidin-3-yl]ethynyl]phenyl)pyrimidine-4-carboxylic acid was reacted with ammonium chloride to give the title compound (8.5 mg, 28%) as a white solid. LC-MS (ES, m/z): 431 [M+H]⁺. ¹H NMR (400 MHz, CD₃OD) δ 8.74 (s, 1H), 8.63 (d, J=8.4 Hz, 1H), 8.32 (s, 1H), 7.69 (d, J=5.8 Hz, 2H), 7.61-7.57 (t, J=7.8 Hz, 1H), 7.17 (s, 1H), 5.00-4.90 (m, 2H), 3.53-3.42 (m, 2H), 2.96 (s, 1H), 2.66-2.60 (m, 1H), 2.39-2.32 (m, 1H), 1.62 (t, J=7.2 Hz, 3H).

### Example NNN

### Synthesis of (R)-2-(3-((3-hydroxy-1-methyl-2-oxopyrrolidin-3-yl)ethynyl)phenyl)-6-(1-methyl-1H-pyrazol-3-yl)pyrimidine-4-carboxamide

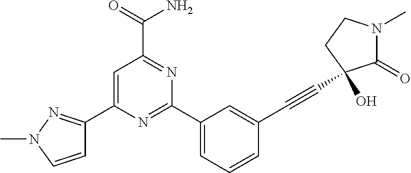

### Step 1: Synthesis of ethyl 2-chloro-6-(1-methyl-1H-pyrazol-3-yl)pyrimidine-4-carboxylate

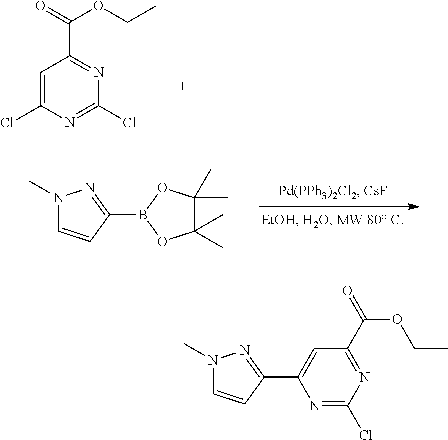

Similar to as described in General Procedure X, ethyl 2,6-dichloropyrimidine-4-carboxylate was reacted with 1-methyl-3-(tetramethyl-1,3,2-dioxaborolan-2-yl)-1H-pyrazole to give the title compound (40.0 mg, 33%) as an off-white solid. LC-MS (ES, m/z): 267 [M+H]⁺.

### Step 2: Synthesis of ethyl 2-(3-[2-[(3R)-3-hydroxy-1-methyl-2-oxopyrrolidin-3-yl]ethynyl]phenyl)-6-(1-methyl-1H-pyrazol-3-yl)pyrimidine-4-carboxylate

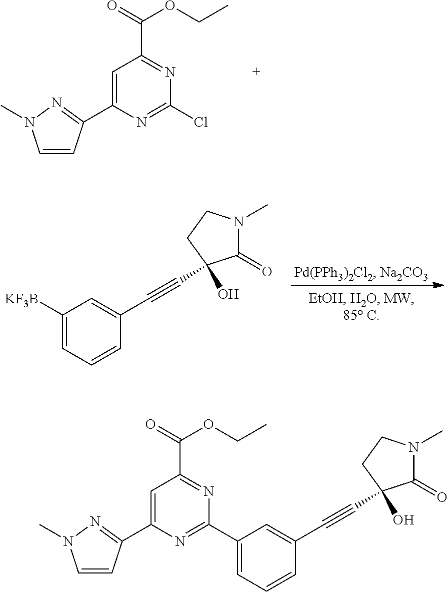

Similar to as described in General Procedure U, ethyl 2-chloro-6-(1-methyl-1H-pyrazol-3-yl)pyrimidine-4-carboxylate was reacted with potassium trifluoro(3-2-[(3R)-3-hydroxy-1-methyl-2-oxopyrrolidin-3-yl]ethynylphenyl)boranideto give the title compound (10.0 mg, 9%) as oil. LC-MS (ES, m/z): 446 [M+H]⁺.

### Step 3: Synthesis of 2-(3-[2-[(3R)-3-hydroxy-1-methyl-2-oxopyrrolidin-3-yl]ethynyl]phenyl)-6-(1-methyl-1H-pyrazol-3-yl)pyrimidine-4-carboxamide

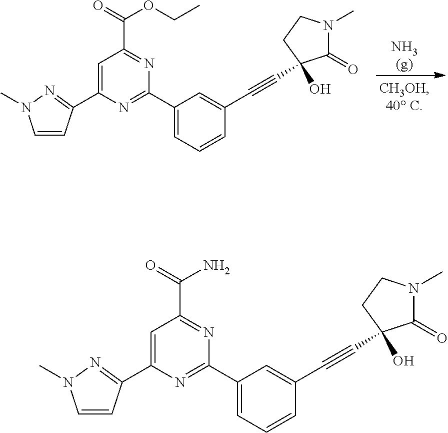 [SMILES: CCOC(=O)c1cc(-c2ccn(C)n2)nc(-c2cccc(C#C[C@]3(O)CCN(C)C3=O)c2)n1 ; CN1CC[C@@](O)(C#Cc2cccc(-c3nc(C(N)=O)cc(-c4ccn(C)n4)n3)c2)C1=O]

Similar to as described in General Procedure S, ethyl 2-(3-[2-[(3R)-3-hydroxy-1-methyl-2-oxopyrrolidin-3-yl]ethynyl]phenyl)-6-(1-methyl-1H-pyrazol-3-yl)pyrimidine-4-carboxylate was reacted with ammonia to give the title compound (3.9 mg, 28%) as a white solid. LC-MS (ES, m/z): 417 [M+H]⁺. ¹H NMR (300 MHz, CD₃OD) δ 8.77 (d, J=1.5 Hz, 1H), 8.68 (d, J=7.8 Hz, 1H), 8.47 (s, 1H), 7.76 (d, J=2.4 Hz, 1H), 7.62-7.54 (m, 2H), 7.19 (d, J=2.1 Hz, 1H), 4.04 (s, 3H), 3.54-3.48 (m, 2H), 2.95 (s, 3H), 2.66-2.60 (m, 1H), 2.39-2.32 (m, 1H).

### Example OOO

### Synthesis of

(R)-2-(3-((3-hydroxy-1-methyl-2-oxopyrrolidin-3-yl)ethynyl)phenyl)-6-(1H-pyrazol-5-yl)pyrimidine-4-carboxamide

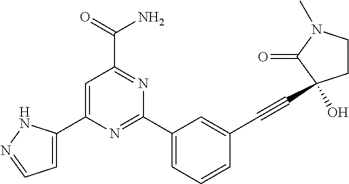 [SMILES: CN1CC[C@@](O)(C#Cc2cccc(-c3nc(C(N)=O)cc(-c4ccn[nH]4)n3)c2)C1=O]

### Step 1: Synthesis of ethyl 6-chloro-2-(3-[2-[(3R)-3-hydroxy-1-methyl-2-oxopyrrolidin-3-yl]ethynyl]phenyl)pyrimidine-4-carboxylate

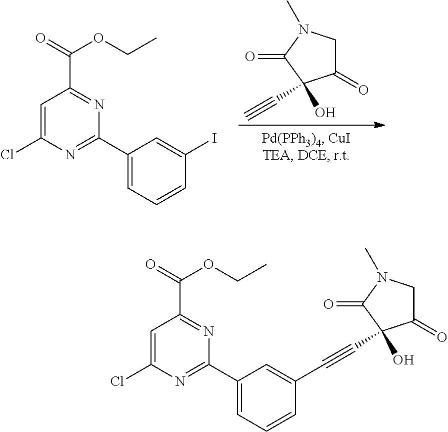

Similar to as described in General Procedure E, ethyl 6-chloro-2-(3-iodophenyl)pyrimidine-4-carboxylate was reacted with (3R)-3-ethynyl-3-hydroxy-1-methylpyrrolidin-2-one to give the title compound (300 mg, 58%) as yellow oil. LC-MS (ES, m/z): 400 [M+H]⁺.

### Step 2: Synthesis of ethyl 2-(3-[2-[(3R)-3-hydroxy-1-methyl-2-oxopyrrolidin-3-yl]ethynyl]phenyl)-6-(1H-pyrazol-5-yl)pyrimidine-4-carboxylate

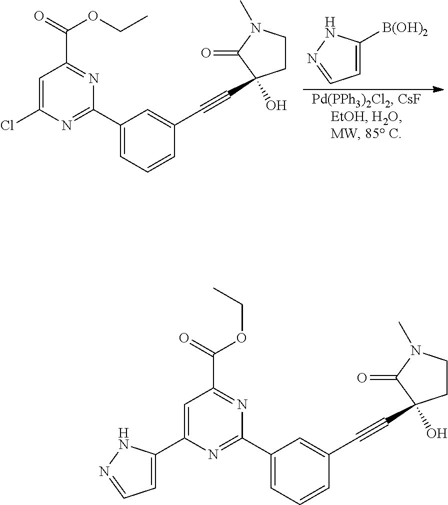

Similar to as described in General Procedure X, (1H-pyrazol-5-yl)boronic acid was reacted with ethyl 6-chloro-2-(3-[2-[(3R)-3-hydroxy-1-methyl-2-oxopyrrolidin-3-yl]ethynyl]phenyl)pyrimidine-4-carboxylate to give the title compound (123 mg, 65.5%) as orange oil. LC-MS (ES, m/z): 432 [M+H]⁺.

### Step 3: Synthesis of 2-(3-[2-[(3R)-3-hydroxy-1-methyl-2-oxopyrrolidin-3-yl]ethynyl]phenyl)-6-(1H-pyrazol-5-yl)pyrimidine-4-carboxamide

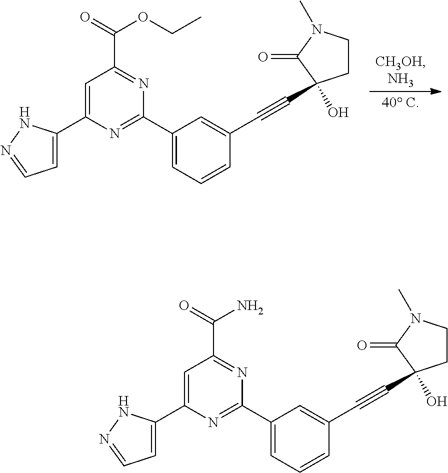

Similar to as described in General Procedure S, ethyl 2-(3-[2-[(3R)-3-hydroxy-1-methyl-2-oxopyrrolidin-3-yl]ethynyl]phenyl)-6-(1H-pyrazol-5-yl)pyrimidine-4-carboxylate was reacted with ammonia to give the title compound (21.1 mg, 38%) as a light yellow solid. LC-MS (ES, m/z): 403 [M+H]⁺. ¹H NMR (300 Mhz, CD₃OD) δ 8.80 (s, 1H), 8.72 (d, J=7.8 Hz, 1H), 8.54 (s, 1H), 7.86 (s, 1H), 7.66 (d, J=7.5 Hz, 1H), 7.57 (t, J=7.8 Hz, 1H), 7.25 (s, 1H), 3.57-3.51 (m, 2H), 2.71-2.63 (m, 1H), 2.42-2.32 (m, 1H).

### Example PPP

### Synthesis of (R)-2-(3-((3-hydroxy-1-methyl-2-oxopyrrolidin-3-yl)ethynyl)phenyl)-6-(1H-imidazol-1-yl)pyrimidine-4-carboxamide

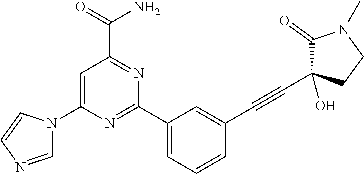 [SMILES: CN1CC[C@@](O)(C#Cc2cccc(-c3nc(C(N)=O)cc(-n4ccnc4)n3)c2)C1=O]

### Step 1: Synthesis of methyl 2-chloro-6-(1H-imidazol-1-yl)pyrimidine-4-carboxylate

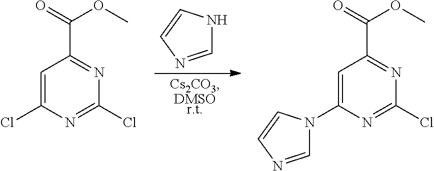

Similar to as described in General Procedure A, methyl 2,6-dichloropyrimidine-4-carboxylate was reacted with 1H-imidazole to give the title compound (140 mg, 24%) as a light yellow solid. LC-MS (ES, m/z): 239 [M+H]⁺.

### Step 2: Synthesis of 2-(3-[2-[(3R)-3-hydroxy-1-methyl-2-oxopyrrolidin-3-yl]ethynyl]phenyl)-6-(1H-imidazol-1-yl)pyrimidine-4-carboxylic acid

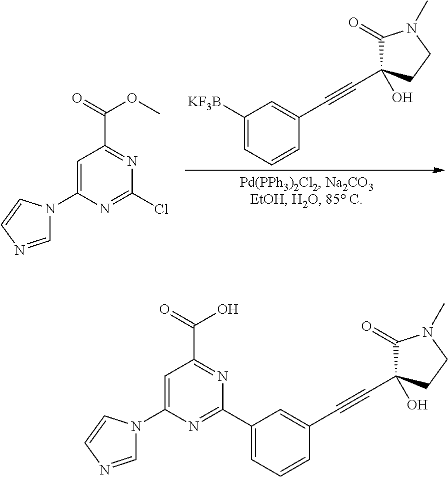

Similar to as described in General Procedure U, methyl 2-chloro-6-(1H-imidazol-1-yl)pyrimidine-4-carboxylate was reacted with potassium trifluoro(3-2-[(3R)-3-hydroxy-1-methyl-2-oxopyrrolidin-3-yl]ethynylphenyl)boranideto give the title compound (210 mg, 83%) as a dark red solid. LC-MS (ES, m/z): 404 [M+H]⁺.

### Step 3: Synthesis of 2-(3-[2-[(3R)-3-hydroxy-1-methyl-2-oxopyrrolidin-3-yl]ethynyl]phenyl)-6-(1H-imidazol-1-yl)pyrimidine-4-carboxamide

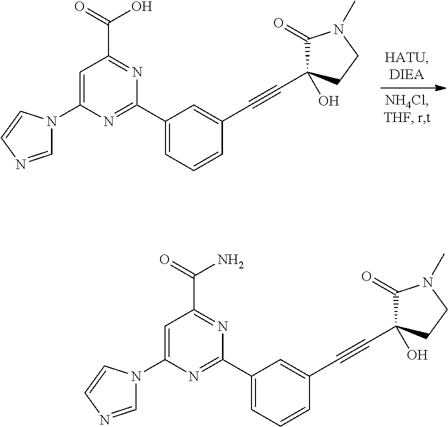 [SMILES: CN1CC[C@@](O)(C#Cc2cccc(-c3nc(C(=O)O)cc(-n4ccnc4)n3)c2)C1=O ; CN1CC[C@@](O)(C#Cc2cccc(-c3nc(C(N)=O)cc(-n4ccnc4)n3)c2)C1=O]

Similar to as described in General Procedure B, 2-(3-[2-[(3R)-3-hydroxy-1-methyl-2-oxopyrrolidin-3-yl]ethynyl]phenyl)-6-(1H-imidazol-1-yl)pyrimidine-4-carb oxylic acid was reacted with ammonium chloride to give the title compound (10 mg, 5%) as an off-white solid. LC-MS (ES, m/z): 403 [M+H]⁺. ¹H NMR (300 MHz, CD₃OD) δ 8.96 (s, 1H), 8.77 (s, 1H), 8.70 (d, J=7.8 Hz, 1H), 8.26 (s, 1H), 8.21 (s, 1H), 7.70 (d, J=7.8 Hz, 1H), 7.60-7.55 (m, 1H), 7.27 (s, 1H), 3.68-3.49 (m, 2H), 2.96 (s, 3H), 2.60-2.68 (m, 1H), 2.39-2.30 (m, 1H).

### Example QQQ

### Synthesis of (R)-2-(3-((3-hydroxy-1-methyl-2-oxopyrrolidin-3-yl)ethynyl)phenyl)-6-(5-methyl-1H-pyrazol-1-yl)pyrimidine-4-carboxamide

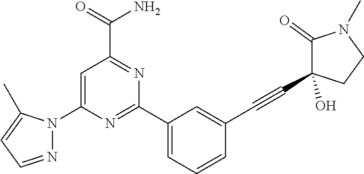 [SMILES: Cc1ccnn1-c1cc(C(N)=O)nc(-c2cccc(C#C[C@]3(O)CCN(C)C3=O)c2)n1]

### Step 1: Synthesis of methyl 2-chloro-6-hydrazinylpyrimidine-4-carboxylate

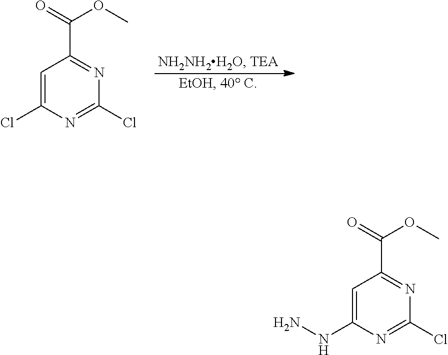

Similar to as described in General Procedure A, hydrazine hydrate was reacted with methyl 2,6-dichloropyrimidine-4-carboxylate to give the title compound (1 g, 51%) as a yellow solid. LC-MS (ES, m/z): 203 [M+H]⁺.

### Step 2: Synthesis of methyl 2-chloro-6-(5-methyl-1H-pyrazol-1-yl)pyrimidine-4-carboxylate

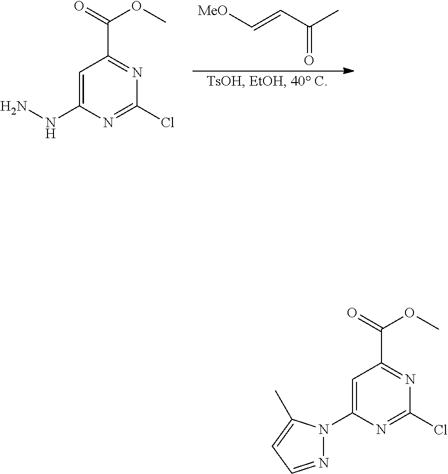

To a solution of methyl 2-chloro-6-hydrazinylpyrimidine-4-carboxylate (300 mg, 1.48 mmol, 1.00 equiv) and 4-methylbenzene-1-sulfonic acid (30 mg, 0.17 mmol, 0.10 equiv) in ethanol (30 mL) was added (3E)-4-methoxybut-3-en-2-one (145 mg, 1.45 mmol, 1.00 equiv). The reaction was heated to 40° C. and stirred at this temperature for 2 hours. The reaction was then cooled to room temperature, concentrated under vacuum, and purified by flash chromatography with ethyl acetate/petroleum ether (1:20). This resulted in 0.32 g (86%) of the title compound as a solid. LC-MS (ES, m/z): 253 [M+H]⁺.

### Step 3: Synthesis of 2-(3-[2-[(3R)-3-hydroxy-1-methyl-2-oxopyrrolidin-3-yl]ethynyl]phenyl)-6-(5-methyl-1H-pyrazol-1-yl)pyrimidine-4-carboxylic acid

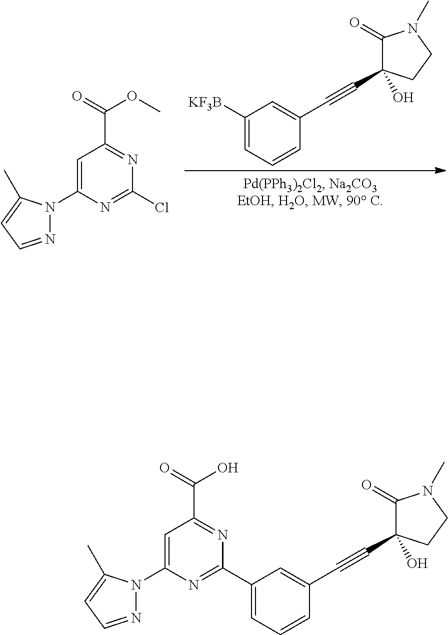

Similar to as described in General Procedure U, methyl 2-chloro-6-(5-methyl-1H-pyrazol-1-yl)pyrimidine-4-carboxylate was reacted with potassium trifluoro(3-2-[(3R)-3-hydroxy-1-methyl-2-oxopyrrolidin-3-yl]ethynylphenyl)boranideto give the title compound (0.15 g, 73%) as a brown solid. LC-MS (ES, m/z): 418 [M+H]⁺.

### Step 4: Synthesis of 2-(3-[2-[(3R)-3-hydroxy-1-methyl-2-oxopyrrolidin-3-yl]ethynyl]phenyl)-6-(5-methyl-1H-pyrazol-1-yl)pyrimidine-4-carboxamide

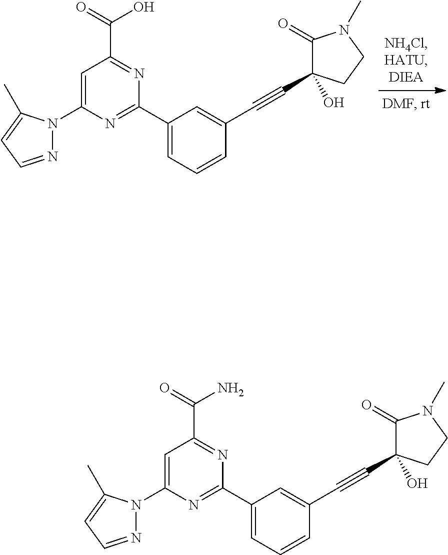 [SMILES: Cc1ccnn1-c1cc(C(=O)O)nc(-c2cccc(C#C[C@]3(O)CCN(C)C3=O)c2)n1 ; Cc1ccnn1-c1cc(C(N)=O)nc(-c2cccc(C#C[C@]3(O)CCN(C)C3=O)c2)n1]

Similar to as described in General Procedure B, 2-(3-[2-[(3R)-3-hydroxy-1-methyl-2-oxopyrrolidin-3-yl]ethynyl]phenyl)-6-(5-methyl-1H-pyrazol-1-yl)pyrimidine-4-carboxylic acid was reacted with ammonium chloride to give the title compound (0.0157 g, 5.4%) as a white solid. LC-MS (ES, m/z): 417 [M+H]⁺. ¹H NMR (400 MHz, CD₃OD) δ 8.71 (s, 1H), 8.40 (s, 1H), 7.6 (s, 1H), 7.67 (d, J=4 Hz, 1H), 7.56 (d, J=4 Hz, 1H), 6.42 (s, 1H), 4.91-3.49 (m, 2H), 2.97 (s, 3H), 2.67-2.61 (m, 1H), 2.42-2.32 (s, 1H).

### Example RRR

### Synthesis of (R)-5-amino-2-(3-((3-hydroxy-1-methyl-2-oxopyrrolidin-3-yl)ethynyl)phenyl)-6-(1H-pyrazol-1-yl)pyrimidine-4-carboxamide

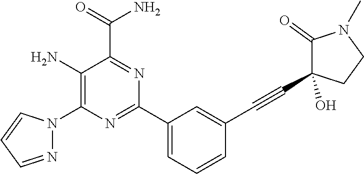 [SMILES: CN1CC[C@@](O)(C#Cc2cccc(-c3nc(C(N)=O)c(N)c(-n4cccn4)n3)c2)C1=O]

### Step 1: Synthesis of ethyl 5-amino-2,6-dichloropyrimidine-4-carboxylate

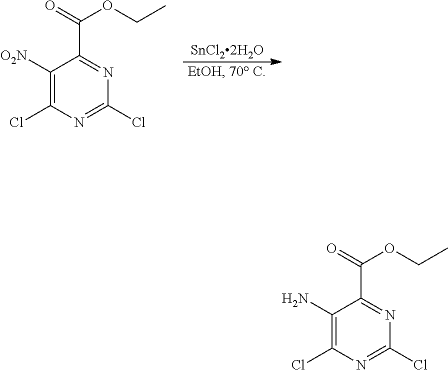 [SMILES: CCOC(=O)c1nc(Cl)nc(Cl)c1[N+](=O)[O-] ; "EtOH, 70° C." ; "SnCl2·2H2O" ; CCOC(=O)c1nc(Cl)nc(Cl)c1N]

A suspension of ethyl 2,6-di chloro-5-nitropyrimidine-4-carboxylate (1 g, 3.76 mmol, 1.00 equiv) and stannous chloride dihydrate (3.38 g, 14.98 mmol, 1.00 equiv) in ethyl acetate (30 mL) in a 50-mL sealed tube was stirred for 5 hours at 70° C. The resulting solution was diluted with 50 mL of water, the pH value of the solution was adjusted to 9 with sodium carbonate, extracted with 3×100 mL of ethyl acetate, washed with water and brine, dried over anhydrous sodium sulfate, and concentrated under vacuum. The residue was purified by a silica gel column with ethyl acetate/petroleum ether (1:5) to give the title compound (572 mg, 64%) as a light yellow solid. LC-MS (ES, m/z): 236 [M+H]⁺.

### Step 2: Synthesis of ethyl 5-amino-2-chloro-6-(1H-pyrazol-1-yl)pyrimidine-4-carboxylate

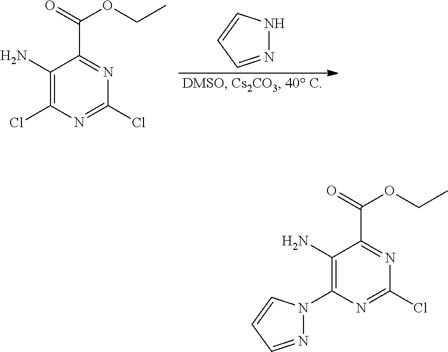

Similar to as described in General Procedure A, ethyl 5-amino-2,6-di chloropyrimidine-4-carboxylate was reacted with 1H-pyrazole to give the title compound (170 mg, 28%) as a light yellow solid. LC-MS (ES, m/z): 268 [M+H]⁺.

### Step 3: Synthesis of methyl 5-amino-2-(3-[2-[(3R)-3-hydroxy-1-methyl-2-oxopyrrolidin-3-yl]ethynyl]phenyl)-6-(1H-pyrazol-1-yl)pyrimidine-4-carboxylate

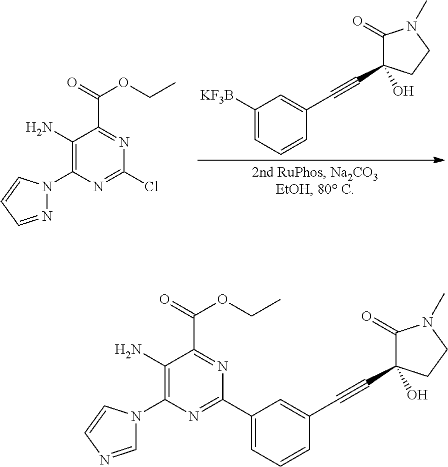

Similar to as described in General Procedure U, ethyl 5-amino-2-chloro-6-(1H-pyrazol-1-yl)pyrimidine-4-carboxylate was reacted with potassium trifluoro(3-2-[(3R)-3-hydroxy-1-methyl-2-oxopyrrolidin-3-yl]ethynylphenyl)boranideto give the title compound (150 mg, 93%) as a light yellow solid. LC-MS (ES, m/z): 461 [M+H]⁺, ¹H NMR (400 MHz, CD₃OD) δ 8.99 (s, 1H), 7.79 (s, 1H), 7.72-7.70 (m, 1H), 7.51-7.40 (m, 2H), 6.30 (s, 1H), 4.45 (s, 1H), 3.58-3.49 (m, 3H), 3.40-3.37 (m, 3H), 2.83 (s, 3H), 2.53-2.47 (m, 1H), 2.26-2.19 (m, 1H), 2.11-2.05 (m, 1H), 1.99-1.96 (m, 1H).

### Step 4: Synthesis of 5-amino-2-(3-[2-[(3R)-3-hydroxy-1-methyl-2-oxopyrrolidin-3-yl]ethynyl]phenyl)-6-(1H-pyrazol-1-yl)pyrimidine-4-carboxamide

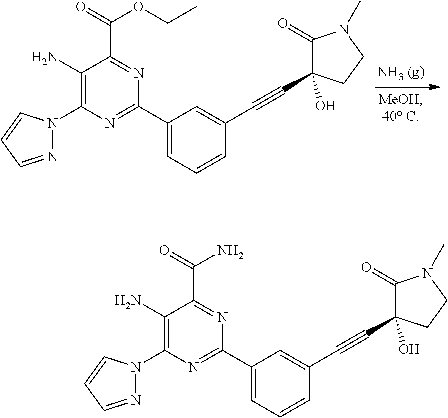 [SMILES: CCOC(=O)c1nc(-c2cccc(C#C[C@]3(O)CCN(C)C3=O)c2)nc(-n2cccn2)c1N ; CN1CC[C@@](O)(C#Cc2cccc(-c3nc(C(N)=O)c(N)c(-n4cccn4)n3)c2)C1=O]

Similar to as described in General Procedure S, ethyl 5-amino-2-(3-[2-[(3R)-3-hydroxy-1-methyl-2-oxopyrrolidin-3-yl]ethynyl]phenyl)-6-(1H-pyrazol-1-yl)pyrimidine-4-carboxylate was reacted with ammonia to give the title compound (19.6 mg, 14%) as a light yellow solid. LC-MS (ES, m/z): 418 [M+H]⁺. ¹H NMR (400 MHz, CD₃OD) δ 8.99 (s, 1H), 8.52 (s, 1H), 8.46 (d, J=7.6 Hz, 1H), 7.93 (s, 1H), 7.53-7.46 (m, 2H), 6.68 (s, 1H), 3.56-3.47 (m, 2H), 2.96 (s, 3H), 2.67-2.61 (m, 1H), 2.38-2.31 (m, 1H).

### Example SSS

### Synthesis of (R)-8-(3-((3-hydroxy-1-methyl-2-oxopyrrolidin-3-yl)ethynyl)phenyl)-1,7-naphthyridine-6-carboxamide

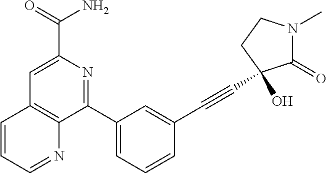 [SMILES: CN1CC[C@@](O)(C#Cc2cccc(-c3nc(C(N)=O)cc4cccnc34)c2)C1=O]

### Step 1: Synthesis of N-tert-butyl-3-methylpyridine-2-carboxamide

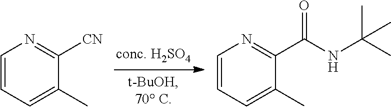

Sulfuric acid (15.0 mL, 281.41 mmol, 2.20 equiv) was added dropwise to a stirred solution of 3-methylpyridine-2-carbonitrile (15.0 g, 126.97 mmol, 1.00 equiv), and tert-butanol (40 mL) at 70° C. The resulting solution was stirred for 30 min at 75° C., diluted with 200 mL of water, and the pH value of the solution was adjusted to 8 with ammonium hydroxide. The resulting mixture was concentrated under vacuum, extracted with ethyl acetate, and concentrated under vacuum. The residue was applied onto a silica gel column with ethyl acetate/petroleum ether (1;20) to give the title compound (21.0 g (86%) as light yellow oil. LC-MS (ES, m/z): 193 [M+H]⁺.

### Step 2: Synthesis of ethyl 3-[2-(tert-butylcarbamoyl)pyridin-3-yl]-2-oxopropanoate

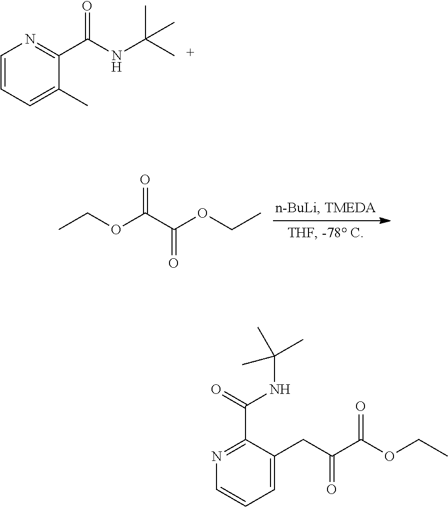

n-BuLi (3.84 g, 59.95 mmol, 2.00 equiv) in hexane (24.0 mL) was added dropwise to a stirred solution of N-tert-butyl-3-methylpyridine-2-carboxamide (5.8 g, 30.17 mmol, 1.00 equiv) in THF (100 mL) at −78° C. under nitrogen. N,N,N′,N′-tetramethylethylenediamine (3.48 g, 1.00 equiv) was then added dropwise and the resulting solution was stirred for 30 min at −78° C. To the mixture a solution of diethyl oxalate (8.76 g, 59.94 mmol, 2.00 equiv) in THF (100 mL) was added dropwise at −78° C. The resulting solution was stirred for additional 1 h at −78° C., quenched by saturated aqueous ammonium chloride, extracted with ethyl acetate, washed with brine, dried over anhydrous sodium sulfate, and concentrated under vacuum. The residue was applied onto a silica gel column with ethyl acetate/petroleum ether (1:5) to give the title compound (2.18 g, 25%) as light yellow oil. LC-MS (ES, m/z): 293 [M+H]⁺.

### Step 3: Synthesis of ethyl 8-oxo-7,8-dihydro-1,7-naphthyridine-6-carboxylate

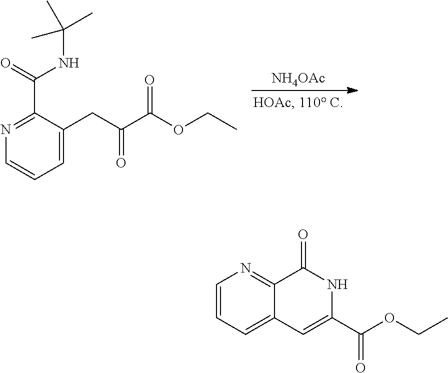 [SMILES: CCOC(=O)C(=O)Cc1cccnc1C(=O)NC(C)(C)C ; CCOC(=O)c1cc2cccnc2c(=O)[nH]1 ; "NH4OAc"]

A solution of ethyl 3-[2-(tert-butylcarbamoyl)pyridin-3-yl]-2-oxopropanoate (1.5 g, 5.13 mmol, 1.00 equiv) and ammonium acetate (790 mg, 10.25 mmol, 2.00 equiv) in acetic acid (15 mL) was stirred for 12 h at 110° C. The solids were collected and dried in an oven under reduced pressure. The residue was applied onto a silica gel column with dichloromethane/methanol (20:1) to give the title compound (0.9 g, 80%) as an off-white solid. LC-MS (ES, m/z): 219 [M+H]⁺.

### Step 4: Synthesis of ethyl 8-chloro-1,7-naphthyridine-6-carboxylate

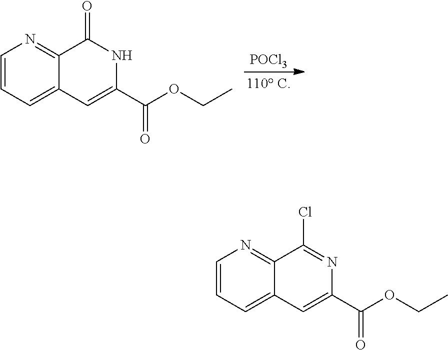 [SMILES: CCOC(=O)c1cc2cccnc2c(=O)[nH]1 ; "POCl3" ; CCOC(=O)c1cc2cccnc2c(Cl)n1]

A solution of ethyl 8-oxo-7,8-dihydro-1, 7-naphthyridine-6-carboxylate (850.0 mg, 3.90 mmol, 1.00 equiv) in phosphorus oxychloride (20.0 mL) was stirred for 30 min at 110° C. The resulting mixture was concentrated under vacuum and the residue was applied onto a silica gel column with dichloromethane/methanol (10:1) to give the title compound (800.0 mg, 87%) as an off-white solid. LC-MS (ES, m/z): 237 [M+H]⁺.

### Step 5: Synthesis of ethyl 8-(3-[2-[(3R)-3-hydroxy-1-methyl-2-oxopyrrolidin-3-yl]ethynyl]phenyl)-1,7-naphthyridine-6-carboxylate

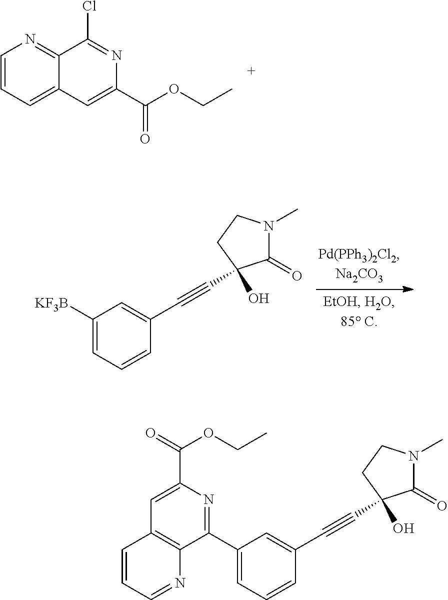

Similar to as described in General Procedure U, ethyl 8-chloro-1,7-naphthyridine-6-carboxylate was reacted with potassium trifluoro(3-2-[(3R)-3-hydroxy-1-methyl-2-oxopyrrolidin-3-yl]ethynylphenyl)boranideto give the title compound (50.0 mg, 24%) as dark red oil. LC-MS (ES, m/z): 416 [M+H]⁺.

### Step 6: Synthesis of 8-(3-[2-[(3R)-3-hydroxy-1-methyl-2-oxopyrrolidin-3-yl]ethynyl]phenyl)-1,7-naphthyridine-6-carboxamide

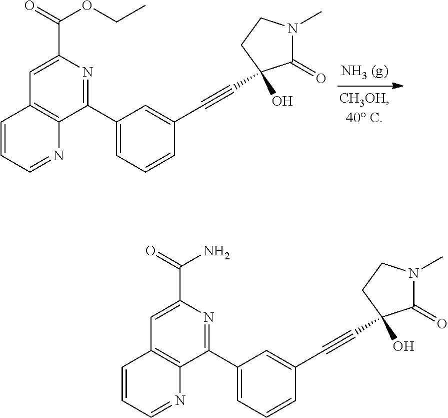 [SMILES: CCOC(=O)c1cc2cccnc2c(-c2cccc(C#C[C@]3(O)CCN(C)C3=O)c2)n1 ; CN1CC[C@@](O)(C#Cc2cccc(-c3nc(C(N)=O)cc4cccnc34)c2)C1=O]

Similar to as described in General Procedure S, ethyl 8-(3-[2-[(3R)-3-hydroxy-1-methyl-2-oxopyrrolidin-3-yl]ethynyl]phenyl)-1,7-naphthyridine-6-carboxylate was reacted with ammonia to give the title compound (17.7 mg, 38%) as a white solid. LC-MS (ES, m/z): 387 [M+H]⁺. ¹H NMR (400 MHz, CD₃OD) δ 9.01 (dd, J=4.0, 1.2 Hz, 1H), 8.51-8.46 (m, 2H), 8.18 (s, 1H), 8.08 (d, J=7.6 Hz, 1H), 7.73 (dd, J=8.4, 4.0 Hz, 1H), 7.51 (d, J=7.6 Hz, 1H), 7.44 (t, J=7.6 Hz, 1H), 3.43-3.34 (m, 2H), 2.83 (s, 3H), 2.54-2.48 (m, 1H), 2.25-2.18 (m, 1H).

### Example TTT

### Synthesis of (R)-4-(3-((3-hydroxy-1-methyl-2-oxopyrrolidin-3-yl)ethynyl)phenyl)-1-methyl-1H-pyrazolo[4,3-c]pyridine-6-carboxamide

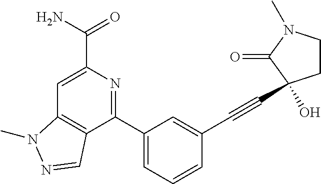 [SMILES: CN1CC[C@@](O)(C#Cc2cccc(-c3nc(C(N)=O)cc4c3cnn4C)c2)C1=O]

### Step 1: Synthesis of 2,4,6-trichloropyridine-3-carbaldehyde

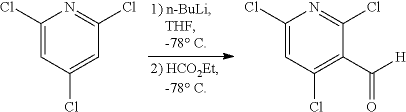 [SMILES: Clc1cc(Cl)nc(Cl)c1 ; O=Cc1c(Cl)cc(Cl)nc1Cl]

n-BuLi (35 mL, 2.5 M in hexanes) was added dropwise to a stirred solution of 2,4,6-trichloropyridine (15 g, 82.22 mmol, 1.00 equiv) in anhydrous THF (200 mL) at −78° C. under nitrogen. After 30 minutes ethyl formate (10 mL, 215.31 mmol) was added at −78° C. The resulting solution was diluted with ethyl acetate, washed with brine, dried over anhydrous sodium sulfate, and concentrated under vacuum. The residue was purified by a silica gel column with ethyl acetate/petroleum ether (1:10) to give the title compound (13 g, 75%) as a white solid. LC-MS (ES, m/z): 210 [M+H]⁺.

### Step 2: Synthesis of 4,6-dichloro-1-methyl-1H-indazole

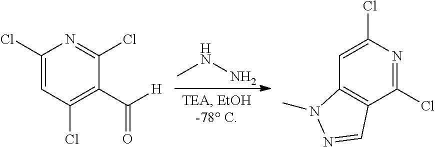

A solution of 2,4,6-trichloropyridine-3-carbaldehyde (3 g, 14.26 mmol, 1.00 equiv) in triethylamine (5.8 mL)/ethanol (100 mL) was cooled to −78° C. and methylhydrazine (1.55 mL, 26.58 mmol, 1.90 equiv) was added slowly. After 30 minutes the resulting solution was concentrated, diluted with ethyl acetate, washed with brine, dried over anhydrous sodium sulfate, and concentrated under vacuum. The residue was purified by a silica gel column with ethyl acetate/petroleum ether (1:10) to give the title compound (1.5 g, 52%) as a yellow solid. LC-MS (ES, m/z): 202 [M+H]⁺.

### Step 3: Synthesis of (3R)-3-[2-(3-[6-chloro-1-methyl-1H-pyrazolo[4,3-c]pyridin-4-yl]phenyl) ethynyl]-3-hydroxy-1-methyl pyrrolidin-2-one

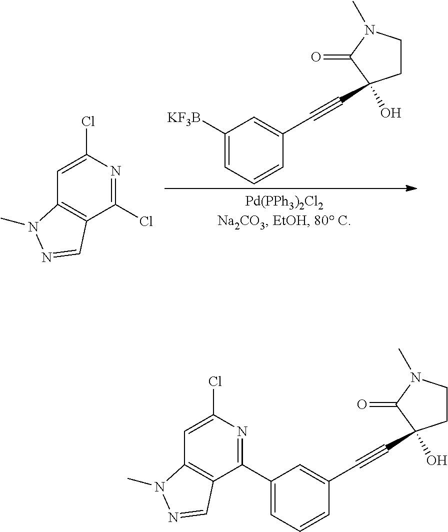

Similar to as described in General Procedure U, 4,6-dichloro-1-methyl-1H-pyrazolo was reacted with potassium trifluoro(3-2-[(3R)-3-hydroxy-1-methyl-2-oxopyrrolidin-3-yl]ethynylphenyl)boranideto give the title compound (152.1 mg, 20 as light yellow oil. LC-MS (ES, m/z): 381 [M+H]⁺.

### Step 4: Synthesis of 4-(3-[2-[(3R)-3-hydroxy-1-methyl-2-oxopyrrolidin-3-yl]ethynyl]phenyl)-1-methyl-1H-pyrazolo[4,3-c]pyridine-6-carboxamide

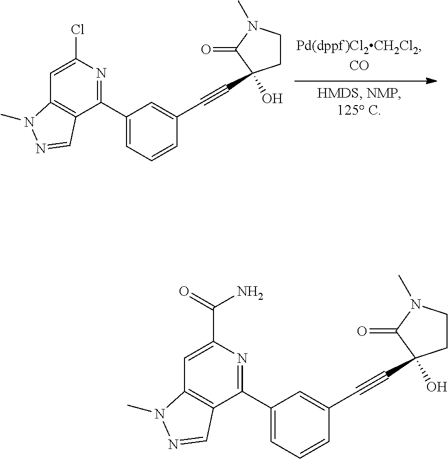 [SMILES: CN1CC[C@@](O)(C#Cc2cccc(-c3nc(Cl)cc4c3cnn4C)c2)C1=O ; CN1CC[C@@](O)(C#Cc2cccc(-c3nc(C(N)=O)cc4c3cnn4C)c2)C1=O]

Similar to as described in General Procedure P, (3R)-3-[2-(3-[6-chloro-1-methyl-1H-pyrazolo[4,3-c]pyridin-4-yl]phenyl)ethynyl]-3-hydroxy-1-methylpyrrolidin-2-one was reacted with carbon monoxide to give the title compound (0.0366 g) as a white solid. LC-MS (ES, m/z): 390 [M+H]⁺. ¹H NMR (400 MHz, CD₃OD) δ 8.49 (s, 1H), 8.33 (s, 1H), 8.28 (s, 1H), 8.20 (d, J=7.6 Hz, 1H), 7.66-7.59 (m, 2H), 4.22 (s, 3H), 3.56-3.48 (m, 2H), 2.99 (s, 3H), 2.67-2.61 (m, 1H), 2.39-2.32 (m, 1H).

### Example UUU

### Synthesis of (R)-8-(3-((3-hydroxy-1-methyl-2-oxopyrrolidin-3-yl)ethynyl)phenyl)-3-methylimidazo[1,2-a]pyrazine-6-carboxamide

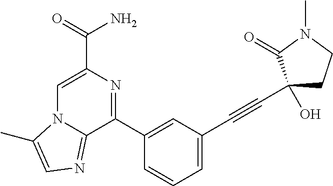 [SMILES: Cc1cnc2c(-c3cccc(C#C[C@]4(O)CCN(C)C4=O)c3)nc(C(N)=O)cn12]

### Step 1: Synthesis of methyl 8-bromo-3-methylimidazo[1,2-a]pyrazine-6-carboxylate

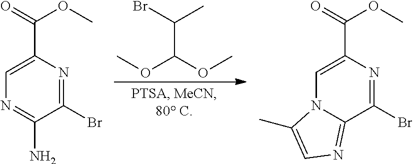

A solution of methyl 5-amino-6-bromopyrazine-2-carboxylate (500 mg, 2.15 mmol, 1.00 equiv), 2-bromo-1,1-dimethoxypropane (1.2 g, 6.56 mmol, 3.00 equiv), p-toluenesulfonic acid (76 mg, 0.44 mmol, 0.20 equiv) in acetonitrile (20 mL) was stirred for 12 h at 80° C. The mixture was concentrated under vacuum and the residue was purified by a silica gel column eluting with dichloromethane/methanol (10:1). This resulted in 400 mg (69%) of the title compound as a yellow solid. LC-MS (ES, m/z): 270 [M+H]⁺.

### Step 2: Synthesis of 8-bromo-3-methylimidazo[1,2-a]pyrazine-6-carboxamide

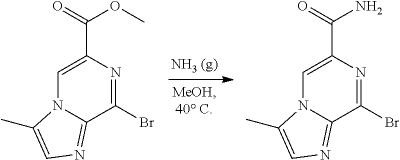

Similar to as described in General Procedure S, 8-bromo-3-methylimidazo[1,2-a]pyrazine-6-carboxylate was reacted with ammonia to give the title compound (380 mg, crude) as a yellow solid. The crude product was directly used in next step without further purification. LC-MS (ES, m/z): 255 [M+H]⁺.

### Step 3: Synthesis of 8-(3-[2-[(3R)-3-hydroxy-1-methyl-2-oxopyrrolidin-3-yl]ethynyl]phenyl)-3-methylimidazo[1,2-a]pyrazine-6-carboxamide

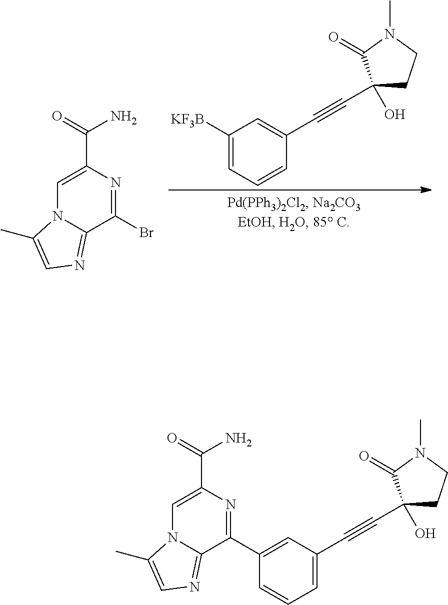

Similar to as described in General Procedure U, 8-bromo-3-methylimidazo[1,2-a]pyrazine-6-carboxamide was reacted with potassium trifluoro(3-2-[(3R)-3-hydroxy-1-methyl-2-oxopyrrolidin-3-yl]ethynylphenyl)boranideto give the title compound (65.2 mg, 11%) as a white solid. LC-MS (ES, m/z): 390 [M+H]⁺. ¹H NMR (400 MHz, CD₃OD) δ 8.88 (s, 1H), 8.81 (s, 3H), 8.69 (d, J=8 Hz, 1H), 7.71 (s, 1H), 7.63 (d, J=7.6 Hz, 1H), 7.55-7.53 (m, 1H), 3.57-3.47 (m, 2H), 2.96 (s, 3H), 2.67-2.62 (m, 4H), 2.39-2.32 (m, 1H).

### Example VVV

### Synthesis of (R)-8-(3-((3-hydroxy-1-methyl-2-oxopyrrolidin-3-yl)ethynyl)phenyl)-2-methylimidazo[1,2-a]pyrazine-6-carboxamide

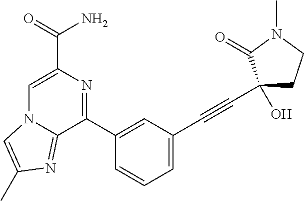 [SMILES: Cc1cn2cc(C(N)=O)nc(-c3cccc(C#C[C@]4(O)CCN(C)C4=O)c3)c2n1]

### Step 1: Synthesis of methyl 8-bromo-2-methylimidazo[1,2-a]pyrazine-6-carboxylate

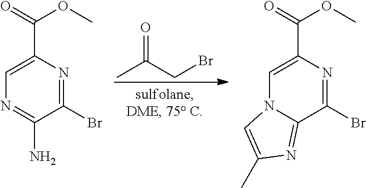

A solution of methyl 5-amino-6-bromopyrazine-2-carboxylate (500 mg, 2.15 mmol, 1.00 equiv), 1-bromopropan-2-one (1.5 g, 10.95 mmol, 5.10 equiv) in sulfolane (10 mL) and ethylene glycol dimethyl ether (10 mL) was stirred for 24 h at 75° C. After cooled to room temperature the mixture was poured into ice-saturated sodium hydrogen carbonate solution and extracted with ethyl acetate. The organic layer was washed with brine, dried over sodium sulfate, and concentrated in vacuum. The residue was purified by silica gel column chromatography eluting with ethyl acetate/petroleum ether (2:3) to afford 140 mg (24%) of the title compound as a yellow solid. LC-MS (ES, m/z): 270 [M+H]⁺.

### Step 2: Synthesis of 8-bromo-2-methylimidazo[1,2-a]pyrazine-6-carboxamide

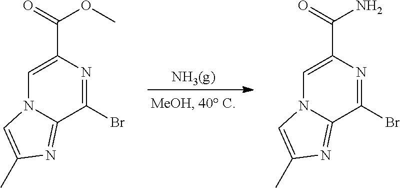

Similar to as described in General Procedure S, methyl 8-bromo-2-methylimidazo[1,2-a]pyrazine-6-carboxylate was reacted with ammonia to give the title compound (200 mg, crude) as a yellow solid. LC-MS (ES, m/z): 255 [M+H]⁺.

### Step 3: Synthesis of 8-(3-[2-[(3R)-3-hydroxy-1-methyl-2-oxopyrrolidin-3-yl]ethynyl]phenyl)-2-methylimidazo[1,2-a]pyrazine-6-carboxamide

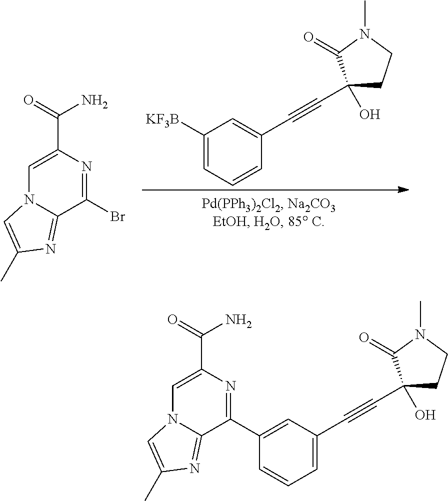

Similar to as described in General Procedure U, 8-bromo-2-methylimidazo[1,2-a]pyrazine-6-carboxamide was reacted with potassium trifluoro(3-2-[(3R)-3-hydroxy-1-methyl-2-oxopyrrolidin-3-yl]ethynylphenyl)boranideto give the title compound (31.6 mg, 10%) as a white solid. LC-MS (ES, m/z): 390 [M+H]⁺. ¹H NMR (400 MHz, CD₃OD) δ 9.07 (s, 1H), 8.77 (s, 1H), 8.70-8.68 (m, 1H), 7.98 (s, 1H), 7.65-7.63 (m, 1H), 7.58-7.54 (m, 1H), 3.56-3.47 (m, 2H), 2.96 (s, 3H), 2.67-2.62 (m, 1H), 5.56 (s, 1H), 2.39-2.32 (m, 1H).

### Example WWW

### Synthesis of (R)-6-(5-((3-hydroxy-1-methyl-2-oxopyrrolidin-3-yl)ethynyl)-2,3-dihydrobenzofuran-7-yl)picolinamide

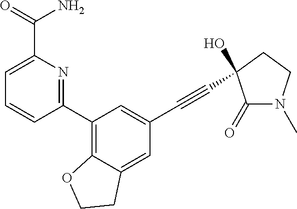 [SMILES: CN1CC[C@@](O)(C#Cc2cc3c(c(-c4cccc(C(N)=O)n4)c2)OCC3)C1=O]

### Step 1: Synthesis of ethyl 6-(5-bromo-2,3-dihydro-1-benzofuran-7-yl)pyridine-2-carboxylate

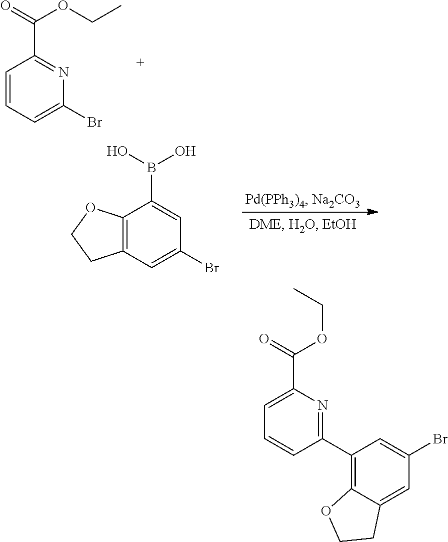

Similar to as described in General Procedure M, ethyl 6-bromopyridine-2-carboxylate was reacted with (5-bromo-2,3-dihydro-1-benzofuran-7-yl)boronic acid to give the title compound (200 mg, 57%) as a yellow solid. LC-MS (ES, m/z): 348, 350 [M+H]⁺.

### Step 2: Synthesis of ethyl 6-(5-[2-[(3R)-3-hydroxy-1-methyl-2-oxopyrrolidin-3-yl]ethynyl]-2,3-dihydro-1-benzofuran-7-yl)pyridine-2-carboxylate

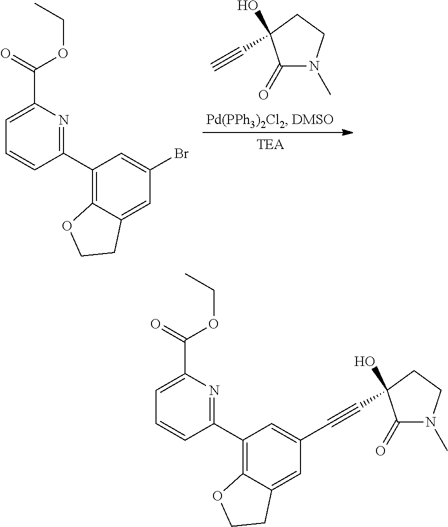

Similar to as described in General Procedure G, ethyl 6-(5-bromo-2,3-dihydro-1-benzofuran-7-yl)pyridine-2-carboxylate was reacted with (3R)-3-ethynyl-3-hydroxy-1-methylpyrrolidin-2-one to give the title compound (200 mg, 86%) as a yellow solid. LC-MS (ES, m/z): 407 [M+H]⁺.

### Step 3: Synthesis of 6-(5-[2-[(3R)-3-hydroxy-1-methyl-2-oxopyrrolidin-3-yl]ethynyl]-2,3-dihydro-1-benzofuran-7-yl)pyridine-2-carboxamide

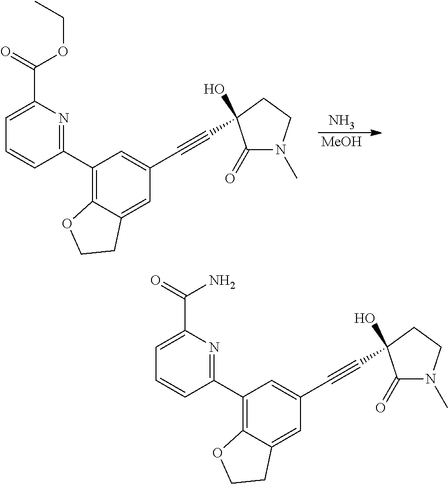

Similar to as described in General Procedure S, ethyl 6-(5-[2-[(3R)-3-hydroxy-1-methyl-2-oxopyrrolidin-3-yl]ethynyl]-2,3-dihydro-1-benzofuran-7-yl)pyridine-2-carboxylate was reacted with ammonia to give the title compound (26.3 mg, 14%) as an off-white solid. LC-MS (ES, m/z): 378 [M+H]⁺. ¹H NMR (400 MHz, CD₃OD) δ 8.39 (d, J=8.8 Hz, 1H), 8.29 (s, 1H), 8.06-7.98 (m, 2H), 7.40 (s, 1H), 4.79-4.74 (m, 2H), 3.54-3.46 (m, 2H), 3.33-3.29 (m, 2H), 2.96 (s, 3H), 2.64-2.58 (m, 1H), 2.36-2.29 (m, 1H).

### Example XXX

### Synthesis of (R)-3-chloro-5-(3-((3-hydroxy-1-methyl-2-oxopyrrolidin-3-yl)ethynyl)phenyl)-1H-pyrrolo[2,3-c]pyridine-7-carboxamide

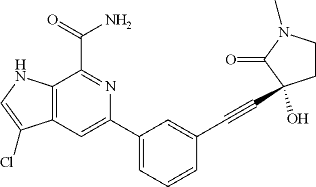 [SMILES: CN1CC[C@@](O)(C#Cc2cccc(-c3cc4c(Cl)c[nH]c4c(C(N)=O)n3)c2)C1=O]

### Step 1: Synthesis of methyl 3,5-di chloro-1-[(4-methylbenzene)sulfonyl]-1H-pyrrolo[2,3-c]pyridine-7-carboxylate

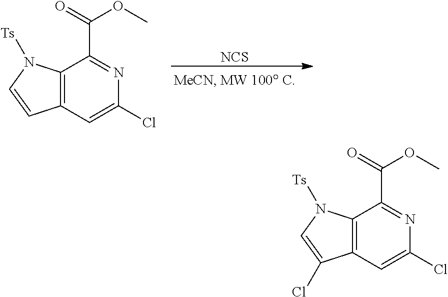 [SMILES: COC(=O)c1nc(Cl)cc2ccn(S(=O)(=O)c3ccc(C)cc3)c12 ; "NCS" ; COC(=O)c1nc(Cl)cc2c(Cl)cn(S(=O)(=O)c3ccc(C)cc3)c12]

A solution of methyl 5-chloro-1-[(4-methylbenzene)sulfonyl]-1H-pyrrolo[2,3-c]pyridine-7-carboxylate (150 mg, 0.41 mmol, 1.00 equiv) and 1-chloropyrrolidine-2,5-dione (82.5 mg, 0.62 mmol, 1.50 equiv) in acetonitrile (15 mL) was irradiated with microwave for 70 minutes at 100° C. The resulting solution was concentrated, diluted with ethyl acetate, washed with brine, dried over anhydrous sodium sulfate, and concentrated under vacuum. The residue was purified by a silica gel column with dichloromethane/methanol (20:1) to give 75 mg (46%) of the title compound as a light yellow solid. LC-MS (ES, m/z): 399 [M+H]⁺.

### Step 2: Synthesis of methyl 3-chloro-5-(3-[2-[(3R)-3-hydroxy-1-methyl-2-oxopyrrolidin-3-yl]ethynyl]phenyl)-1-[(4-methylbenzene)sulfonyl]-1H-pyrrolo[2,3-c]pyridine-7-carboxylate

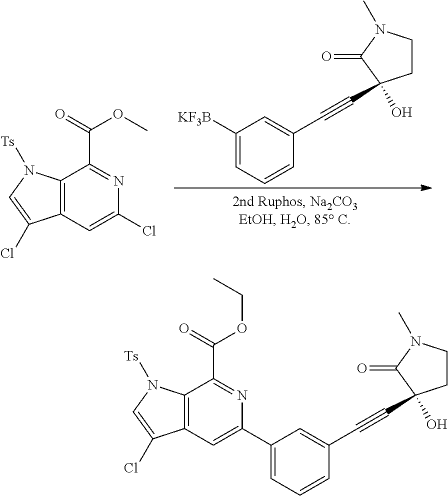

Similar to as described in General Procedure U, methyl 3,5-dichloro-1-[(4-methylbenzene)sulfonyl]-1H-pyrrolo[2,3-c]pyridine-7-carboxylate was reacted with potassium trifluoro(3-2-[(3R)-3-hydroxy-1-methyl-2-oxopyrrolidin-3-yl]ethynylphenyl)boranideto give the title compound (110 mg, 38%) as a light brown solid. LC-MS (ES, m/z): 592 [M+H]⁺.

### Step 3: Synthesis of 3-chloro-5-(3-[2-[(3R)-3-hydroxy-1-methyl-2-oxopyrrolidin-3-yl]ethynyl]phenyl)-1H-pyrrolo[2,3-c]pyridine-7-carboxamide

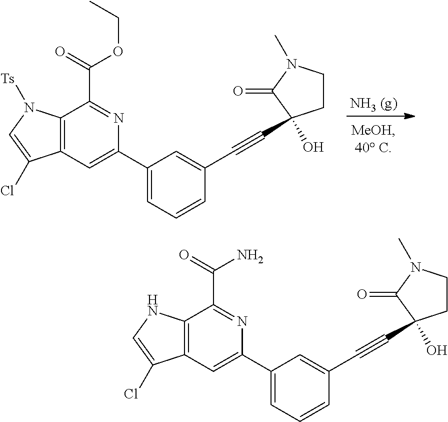

Similar to as described in General Procedure S, methyl 3-chloro-5-(3-[2-[(3R)-3-hydroxy-1-methyl-2-oxopyrrolidin-3-yl]ethynyl]phenyl)-1-[(4-methylbenzene)sulfonyl]-1H-pyrrolo[2,3-c]pyridine-7-carboxylate was reacted with ammonia to give the title compound (42.3 mg, 50%) as a white solid. LC-MS (ES, m/z): 409 [M+H]⁺. ¹H NMR (300 MHz, DMSO-d₆) δ11.84 (s, 1H), 8.54 (s, 1H), 8.39 (d, J=7.5 Hz, 1H), 8.32 (s, 1H), 8.27 (s, 1H), 7.86 (s, 1H), 7.74 (s, 1H), 7.53-7.42 (m, 2H), 6.47 (s, 1H), 3.40-3.35 (m, 2H), 2.81 (s, 3H), 2.53-2.45 (m, 1H), 2.25-2.08 (m, 1H).

### Example YYY

### Synthesis of (R)-5-fluoro-6-(3-((3-hydroxy-1-methyl-2-oxopyrrolidin-3-yl)ethynyl)phenyl)-3-(oxetan-3-ylamino)picolinamide

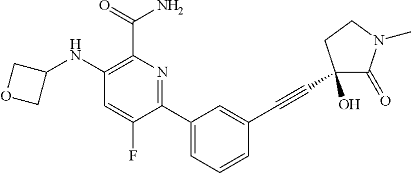 [SMILES: CN1CC[C@@](O)(C#Cc2cccc(-c3nc(C(N)=O)c(NC4COC4)cc3F)c2)C1=O]

### Step 1: Synthesis of methyl 5-fluoro-3-iodopyridine-2-carboxylate

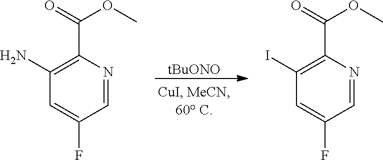

A mixture of methyl 3-amino-5-fluoropyridine-2-carboxylate (800 mg, 4.70 mmol, 1.00 equiv), tert-butyl nitrite (730 mg, 7.08 mmol, 1.50 equiv), and copper (I) iodide (1.3 g, 6.83 mmol, 1.50 equiv) in acetonitrile (20 mL) was stirred for 2 h at 60° C. The mixture was diluted with water, extracted with ethyl acetate. dried over anhydrous sodium sulfate, and concentrated under vacuum. The residue was purified by silica gel column chromatography eluting with ethyl acetate/petroleum ether (5:1) to afford 770 mg (58%) of the title compound as yellow oil. LC-MS (ES, m/z): 282 [M+H]⁺.

### Step 2: Synthesis of methyl 5-fluoro-3-[(oxetan-3-yl)amino]pyridine-2-carboxylate

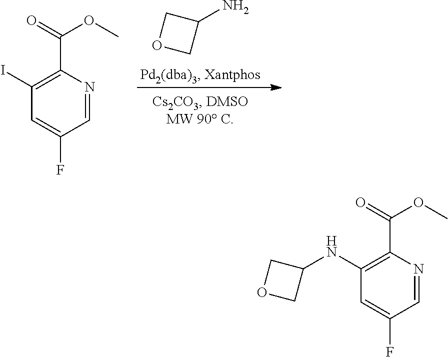

A suspension of methyl 5-fluoro-3-iodopyridine-2-carboxylate (700 mg, 2.49 mmol, 1.00 equiv), oxetan-3-amine (912 mg, 12.48 mmol, 5.00 equiv), Pd₂(dba)₃ (229 mg, 0.25 mmol, 0.10 equiv), Xantphos (290 mg, 0.50 mmol, 0.20 equiv), and cesium carbonate (1.6 g, 4.91 mmol, 2.00 equiv) in DMSO (15 mL) was heated with microwave radiation at 90° C. for 1 h. After cooling the mixture was diluted with dichloromethane, washed with water and brine, dried over anhydrous sodium sulfate, and concentrated under vacuum. The residue was purified by silica gel column chromatography eluting with dichloromethane/methanol (10:1) to afford the title compound (310 mg, 55%) as a yellow solid. LC-MS (ES, m/z): 227 [M+H]⁺.

### Step 3: Synthesis of methyl 6-bromo-5-fluoro-3-[(oxetan-3-yl)amino]pyridine-2-carboxylate

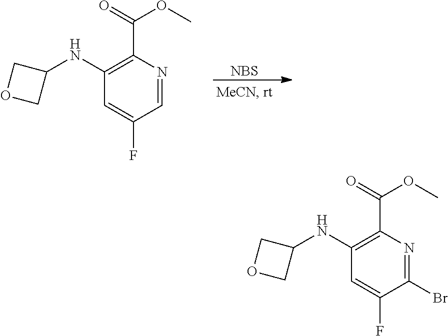

A solution of methyl 5-fluoro-3-[(oxetan-3-yl)amino]pyridine-2-carboxylate (150 mg, 0.66 mmol, 1.00 equiv) and N-bromosuccinimide (142 mg, 0.80 mmol, 1.20 equiv) in acetonitrile (20 mL) was stirred for 12 h at 25° C. The mixture was concentrated under vacuum to afford a crude product which was used directly to the next step without further purification. LC-MS (ES, m/z): 305 [M+H]⁺.

### Step 4: Synthesis of 6-bromo-5-fluoro-3-[(oxetan-3-yl)amino]pyridine-2-carboxamide

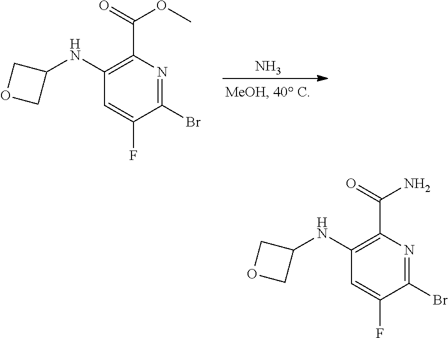

Similar to as described in General Procedure S, methyl 6-bromo-5-fluoro-3-[(oxetan-3-yl)amino]pyridine-2-carboxylate was reacted with ammonia to give the title compound (145 mg, 95%) as a yellow solid. LC-MS (ES, m/z): 290 [M+H]⁺.

### Step 5: Synthesis of 5-fluoro-6-(3-[2-[(3R)-3-hydroxy-1-methyl-2-oxopyrrolidin-3-yl]ethynyl]phenyl)-3-[(oxetan-3-yl)amino]pyridine-2-carboxamide

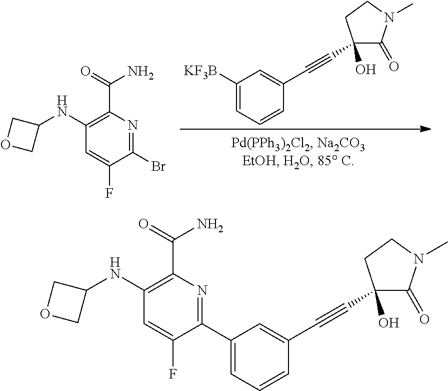

Similar to as described in General Procedure U, 6-bromo-5-fluoro-3-[(oxetan-3-yl)amino]pyridine-2-carboxamide was reacted with potassium trifluoro(3-2-[(3R)-3-hydroxy-1-methyl-2-oxopyrrolidin-3-yl]ethynylphenyl)boranideto give the title compound (40.6 mg, 19%) as a white solid. LC-MS (ES, m/z): 425 [M+H]⁺. ¹H NMR (400 MHz, DMSO-d₆) δ 9.17 (d, J=4.8 Hz, 1H), 8.15 (s, 1H), 8.04-8.02 (m, 1H), 7.93 (s, 1H), 7.68 (s, 1H), 7.50-7.42 (m, 2H), 7.05 (d, J=14.0 Hz, 1H), 6.48 (s, 1H), 4.94-4.91 (m, 2H), 4.78-4.72 (m, 1H), 4.46-4.43 (m, 2H), 3.37-3.33 (m, 2H), 2.80 (s, 3H), 2.47-2.42 (m, 1H), 2.22-2.15 (m, 1H).

### Example ZZZ

### Synthesis of (R)-5-fluoro-6-(3-((3-hydroxy-1-methyl-2-oxopyrrolidin-3-yl)ethynyl)phenyl)-3-((2-methoxyethyl)amino)picolinamide

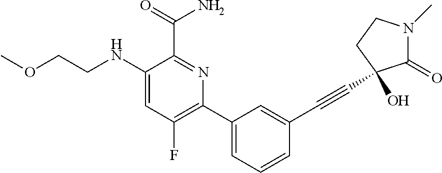 [SMILES: COCCNc1cc(F)c(-c2cccc(C#C[C@]3(O)CCN(C)C3=O)c2)nc1C(N)=O]

### Step 1: Synthesis of N-(2-bromo-5-fluoropyridin-3-yl)-2-methoxyacetamide

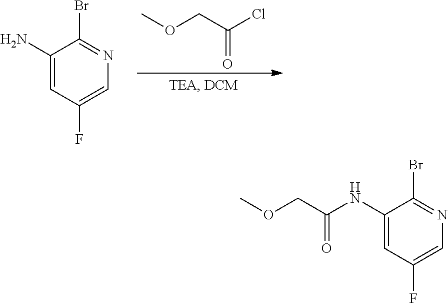

A solution of 2-methoxyacetyl chloride (5.20 g, 47.92 mmol, 6.00 equiv) in dichloromethane (10 mL) was added dropwise to a stirred solution of 2-bromo-5-fluoropyridin-3-amine (1.50 g, 7.85 mmol, 1.00 equiv) in dichloromethane (100 mL) and triethylamine (6.65 g, 65.72 mmol, 8.00 equiv) at 0° C. under nitrogen. The resulting solution was stirred for 16 hours at room temperature and concentrated under vacuum. The residue was applied onto a silica gel column with ethyl acetate/petrol ether (1:5). This resulted in 1.9 g (92%) of the title compound as a yellow solid. LC-MS (ES, m/z): 263 [M+H]⁺.

### Step 2: Synthesis of 2-bromo-5-fluoro-N-(2-methoxyethyl)pyridin-3-amine

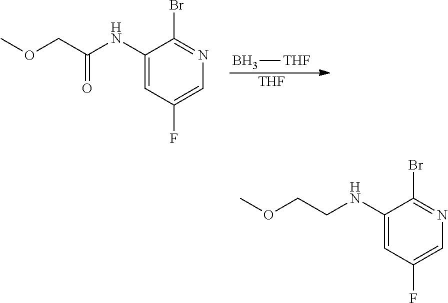

A solution of N-(2-bromo-5-fluoropyridin-3-yl)-2-methoxyacetamide (1.9 g, 7.22 mmol, 1.00 equiv) in THF (50 mL) and BH₃-THF (22.8 mL, 236.12 mmol, 3.00 equiv) was stirred for 4 h at room temperature. The resulting mixture was concentrated under vacuum and the residue was dissolved in ethyl acetate. The reaction was then quenched by aqueous ammonium chloride. The concentrated residue was applied onto a silica gel column with ethyl acetate/petrol ether (1:5) to give the title compound 0.8 g (44%) as yellow oil. LC-MS (ES, m/z): 249 [M+H]⁺.

### Step 3: Synthesis of methyl 5-fluoro-3-[(2-methoxyethyl)amino]pyridine-2-carboxylate

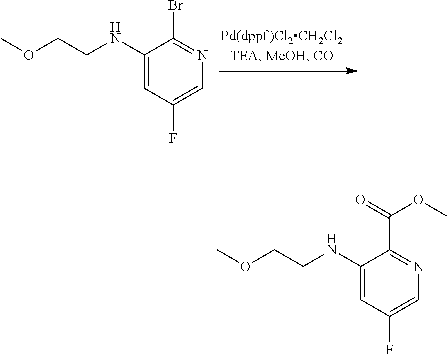

Similar to as described in General Procedure O, 2-bromo-5-fluoro-N-(2-methoxyethyl)pyridin-3-amine was reacted with carbon monoxide to give the title compound (110 mg, 65%) as yellow oil. LC-MS (ES, m/z): 229 [M+H]⁺.

### Step 4: Synthesis of methyl 6-bromo-5-fluoro-3-[(2-methoxyethyl)amino]pyridine-2-carboxylate

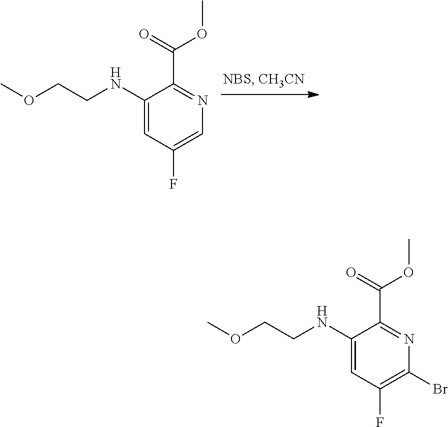

A solution of methyl 5-fluoro-3-[(2-methoxyethyl)amino]pyridine-2-carboxylate (110 mg, 0.48 mmol, 1.00 equiv) in acetonitrile (10 mL) and NBS (98.5 mg, 0.55 mmol, 1.10 equiv) was stirred for 16 hours at room temperature. The resulting mixture was concentrated under vacuum and the residue was applied onto a silica gel column with ethyl acetate/petrol ether (1:4) to give the title compound (150 mg, 86%) as a yellow solid. LC-MS (ES, m/z): 307 [M+H]⁺.

### Step 5: Synthesis of 6-bromo-5-fluoro-3-[(2-methoxyethyl)amino]pyridine-2-carboxamide

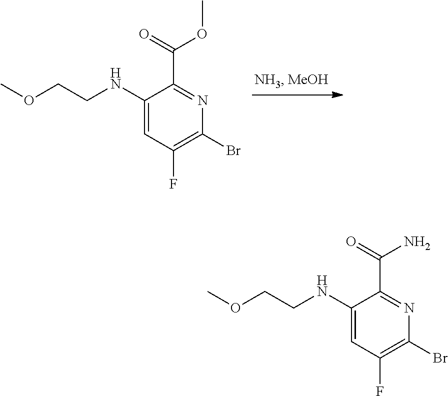 [SMILES: COCCNc1cc(F)c(Br)nc1C(=O)OC ; COCCNc1cc(F)c(Br)nc1C(N)=O]

Similar to as described in General Procedure S, methyl 6-bromo-5-fluoro-3-[(2-methoxyethyl)amino]pyridine-2-carboxylate was reacted with ammonia to give the title compound (110 mg, 93%) as a yellow solid. LC-MS (ES, m/z): 292 [M+H]⁺.

### Step 6: Synthesis of 5-fluoro-6-(3-[2-[(3R)-3-hydroxy-1-methyl-2-oxopyrrolidin-3-yl]ethynyl]phenyl)-3-[(2-methoxyethyl)amino]pyridine-2-carboxamide

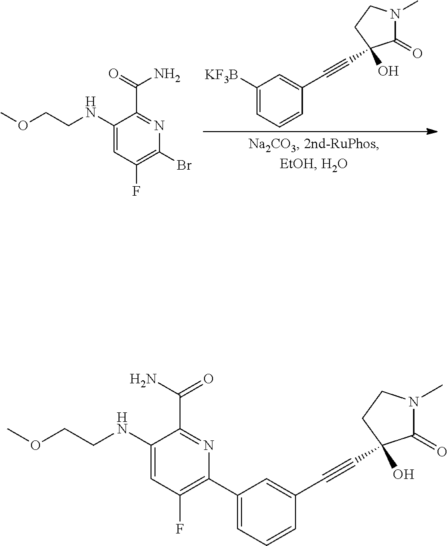

Similar to as described in General Procedure U, 6-bromo-5-fluoro-3-[(2-methoxyethyl)amino]pyridine-2-carboxamide was reacted with potassium trifluoro(3-2-[(3R)-3-hydroxy-1-methyl-2-oxopyrrolidin-3-yl]ethynylphenyl)boranideto give the title compound (32.2 mg, 22%) as an off-white solid. LC-MS (ES, m/z): 427 [M+H]⁺. ¹H NMR (400 MHz, CDCl₃) δ 8.66 (s, 1H), 7.76 (t, J=9.0 Hz, 3H), 7.39-7.26 (m, 2H), 6.80 (d, J=10.2 Hz, 3H), 6.12 (s, 1H), 4.45 (s, 1H), 6.63 (s, 2H), 3.51-3.36 (m, 7H), 2.97 (s, 3H), 2.68-2.65 (m, 1H), 2.39-2.30 (m, 1H).

### Example AAAA and BBBB

### Synthesis of 5-fluoro-6-(3-(((R)-3-hydroxy-1-methyl-2-oxopyrrolidin-3-yl)ethynyl)phenyl)-3-(((R)-oxetan-2-ylmethyl)amino)picolinamide and 5-fluoro-6-(3-(((R)-3-hydroxy-1-methyl-2-oxopyrrolidin-3-yl)ethynyl)phenyl)-3-(((S)-oxetan-2-ylmethyl)amino)picolinamide

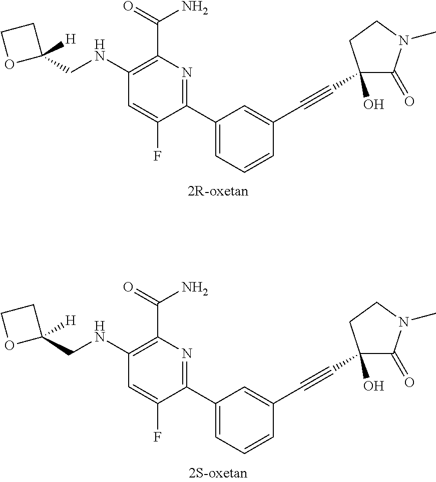 [SMILES: CN1CC[C@@](O)(C#Cc2cccc(-c3nc(C(N)=O)c(NC[C@H]4CCO4)cc3F)c2)C1=O ; CN1CC[C@@](O)(C#Cc2cccc(-c3nc(C(N)=O)c(NC[C@@H]4CCO4)cc3F)c2)C1=O]

### Step 1: Synthesis of methyl 5-fluoro-3-iodopyridine-2-carboxylate

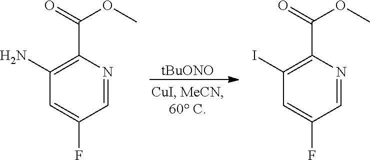

To a solution of methyl 3-amino-5-fluoropyridine-2-carboxylate (800 mg, 4.70 mmol, 1.00 equiv) in MeCN (20 mL) was added tBuONO (730 mg, 7.08 mmol, 1.50 equiv) and CuI (1.3 g, 6.83 mmol, 1.50 equiv) under nitrogen. The reaction mixture was stirred for 1.5 h at 60° C. and concentrated under vacuum. The residue was purified by a silica gel column with ethyl acetate/petroleum ether (1:15) to give the title compound (460 mg, 35%) as yellow oil. LC-MS (ES, m/z): 281 [M+H]⁺.

### Step 2: Synthesis of methyl 5-fluoro-3-[(oxetan-2-ylmethyl)amino]pyridine-2-carboxylate

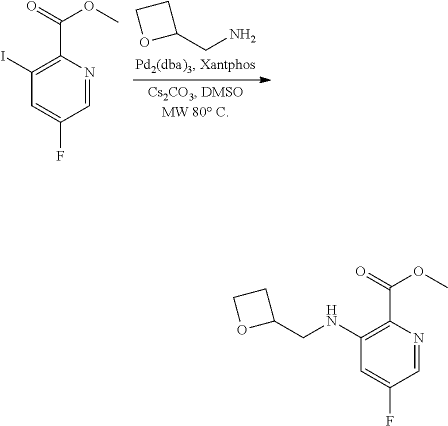

A mixture of methyl 5-fluoro-3-iodopyridine-2-carboxylate (300 mg, 1.07 mmol, 1.00 equiv) in DMSO (20 mL), oxetan-2-ylmethanamine (185 mg, 2.12 mmol, 2.00 equiv), Xantphos (123 mg, 0.21 mmol, 0.20 equiv) and Cs₂CO₃ (700 mg, 2.15 mmol, 2.00 equiv) was irradiated with microwave for 70 minutes at 80° C. The reaction was then diluted with ethyl acetate, washed with brine, and concentrated under vacuum. The residue was purified by a silica gel column with ethyl acetate/petroleum ether (1:5) to give the title compound (45 mg, 18%) as orange oil. LC-MS (ES, m/z): 241 [M+H]⁺.

### Step 3: Synthesis of methyl 6-bromo-5-fluoro-3-[(oxetan-2-ylmethyl)amino]pyridine-2-carboxylate

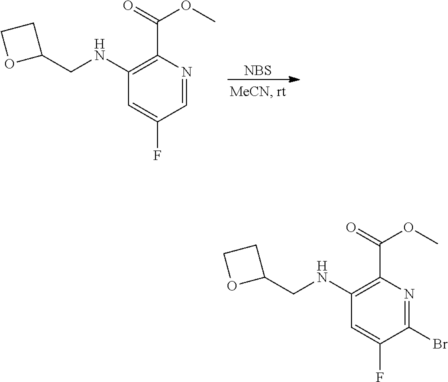

To a solution of methyl 5-fluoro-3-[(oxetan-2-ylmethyl)amino]pyridine-2-carboxylate (180 mg, 0.75 mmol, 1.00 equiv) in acetonitrile (20 mL) was added NBS (160 mg, 0.90 mmol, 1.20 equiv). The reaction mixture was stirred for 2 hours at 25° C. and concentrated under vacuum. The residue was purified by a silica gel column with ethyl acetate/petroleum ether (1:10) to give the title compound (0.2 g, 84%) as a yellow solid. LC-MS (ES, m/z): 319 [M+H]⁺.

### Step 4: Synthesis of 6-bromo-5-fluoro-3-[(oxetan-2-ylmethyl)amino]pyridine-2-carboxamide

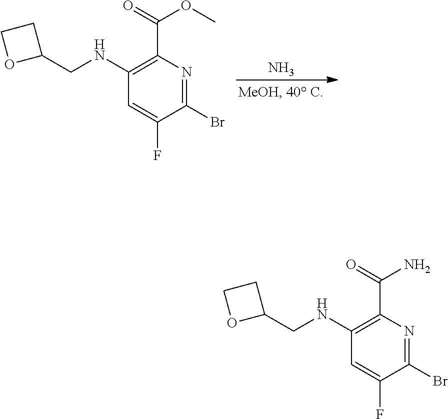

Similar to as described in General Procedure S, methyl 6-bromo-5-fluoro-3-[(oxetan-2-ylmethyl)amino]pyridine-2-carboxylate was reacted with ammonia to give the title compound (0.2 g, 95%) as a yellow solid. LC-MS (ES, m/z): 304 [M+H]⁺.

### Step 5: Synthesis of 5-fluoro-6-(3-(((R)-3-hydroxy-1-methyl-2-oxopyrrolidin-3-yl)ethynyl)phenyl)-3-(((R)-oxetan-2-ylmethyl)amino)picolinamide and 5-fluoro-6-(3-(((R)-3-hydroxy-1-methyl-2-oxopyrrolidin-3-yl)ethynyl)phenyl)-3-(((S)-oxetan-2-ylmethyl)amino)picolinamide

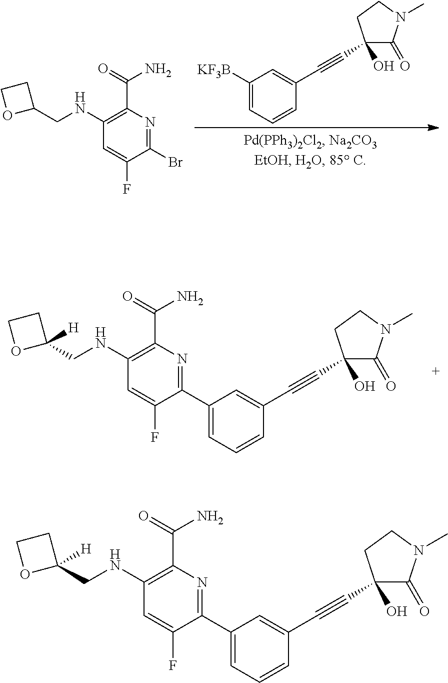

Similar to as described in General Procedure U, 6-bromo-5-fluoro-3-[(oxetan-2-ylmethyl)amino]pyridine-2-carboxamide was reacted with potassium trifluoro(3-2-[(3R)-3-hydroxy-1-methyl-2-oxopyrrolidin-3-yl]ethynylphenyl)boranideto give the 2S-oxetane isomer (0.019 g, 4%) as a white solid and the 2R-oxetane isomer (0.002 g, 0.4%) as a white solid. The stereochemistry of both isomers was arbitrarily assigned. 2R-oxetan isomer: t_{R}=7.98 min (CHIRALPAK IA-3, 0.46*5 cm, Hex:EtOH=50:50, 1.0 ml/min); 2S-oxetan isomer: t_{R}=3.98 min (CHIRALPAK IA-3, 0.46*5 cm, Hex:EtOH=50:50, 1.0 ml/min); Both isomers showed identical LC-MS and ¹H NMR as shown below. LC-MS (ES, m/z): 439 [M+H]⁺. ¹H NMR (DMSO-d₆) δ 9.01 (s, 1H), 8.08-8.01 (m, 2H), 7.93 (s, 1H), 7.58 (s, 1H), 7.50-7.30 (m, 3H), 6.48 (s, 1H), 4.95-4.93 (m, 1H), 4.55-4.44 (m, 2H), 3.52-3.47 (m, 2H), 3.37-3.30 (m, 2H), 2.80 (s, 3H), 2.72-2.64 (m, 1H), 2.50-2.41 (m, 2H), 2.23-2.16 (m, 1H).

### Example CCCC

### Synthesis of (R)-5-fluoro-6-(3-((3-hydroxy-1-methyl-2-oxopyrrolidin-3-yl)ethynyl)phenyl)-3-((2,2,2-trifluoroethyl)amino)picolinamide

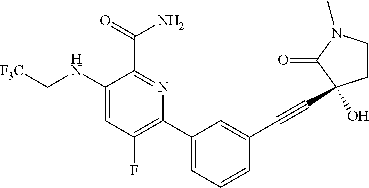 [SMILES: CN1CC[C@@](O)(C#Cc2cccc(-c3nc(C(N)=O)c(NCC(F)(F)F)cc3F)c2)C1=O]

### Step 1: Synthesis of methyl 6-bromo-5-fluoro-3-[(2,2,2-trifluoroethyl)amino]pyridine-2-carboxylate

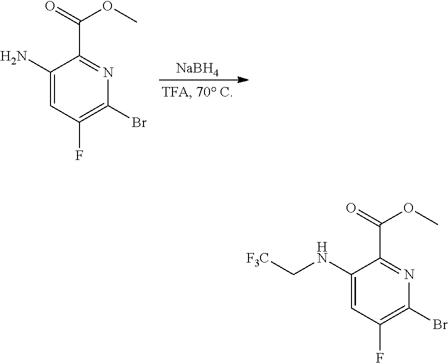 [SMILES: COC(=O)c1nc(Br)c(F)cc1N ; "NaBH4" ; COC(=O)c1nc(Br)c(F)cc1NCC(F)(F)F]

To a solution of methyl 3-amino-6-bromo-5-fluoropyridine-2-carboxylate (400 mg, 1.61 mmol, 1.00 equiv) in trifluoroacetic acid (10 mL) was added sodium borohydride (200 mg, 5.29 mmol, 3.30 equiv) in portions. After being stirred at 70° C. for 12 hours the reaction was allowed to cool to room temperature and quenched by 20 mL of water. The resulting solution was extracted with ethyl acetate, washed with brine, dried over anhydrous sodium sulfate, and concentrated under vacuum. The residue was applied onto a silica gel column with ethyl acetate/petroleum ether (1:10) to give the title compound (260 mg, 49%) as a white solid. LC-MS (ES, m/z): 331, 333 [M+H]⁺.

### Step 2: Synthesis of 6-bromo-5-fluoro-3-[(2,2,2-trifluoroethyl)amino]pyridine-2-carboxamide

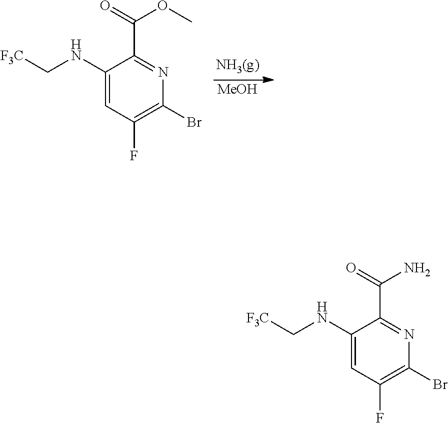 [SMILES: "MeOH" ; COC(=O)c1nc(Br)c(F)cc1NCC(F)(F)F ; NC(=O)c1nc(Br)c(F)cc1NCC(F)(F)F]

Similar to as described in General Procedure S, methyl 6-bromo-5-fluoro-3-[(2,2,2-trifluoroethyl)amino]pyridine-2-carboxylate was reacted with ammonia to give the title compound (180 mg, 99%) as a white solid. LC-MS (ES, m/z): 316, 318 [M+H]⁺.

### Step 3: Synthesis of 5-fluoro-6-(3-[2-[(3R)-3-hydroxy-1-methyl-2-oxopyrrolidin-3-yl]ethynyl]phenyl)-3-[(2,2,2-trifluoroethyl)amino]pyridine-2-carboxamide

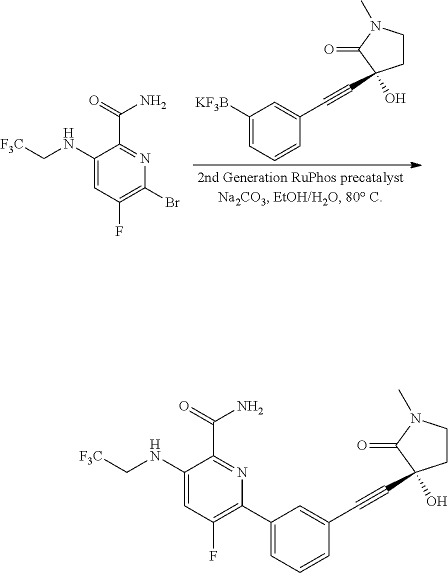

Similar to as described in General Procedure U, 6-bromo-5-fluoro-3-[(2,2,2-trifluoroethyl)amino]pyridine-2-carboxamide was reacted with potassium trifluoro(3-2-[(3R)-3-hydroxy-1-methyl-2-oxopyrrolidin-3-yl]ethynylphenyl)boranideto give the title compound (67.1 mg, 28%) as a white solid. LC-MS (ES, m/z): 451 [M+H]⁺. ¹H NMR (400 MHz, CD₃OD) δ 8.03 (s, 1H), 7.96 (dd, J=6.8, 1.6 Hz, 1H), 7.50-7.45 (m, 2H), 7.35 (d, J=13.6 Hz, 1H), 4.13 (q, J=9.2 Hz, 2H), 3.53-3.47 (m, 2H), 2.95 (s, 3H), 2.65-2.59 (m, 1H), 2.37-2.30 (m, 1H).

### Example DDDD

### Synthesis of (R)-3-amino-6-(2-fluoro-5-((3-hydroxy-1-methyl-2-oxopyrrolidin-3-yl)ethynyl)phenyl)picolinamide

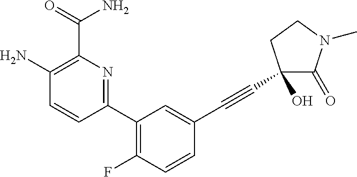 [SMILES: CN1CC[C@@](O)(C#Cc2ccc(F)c(-c3ccc(N)c(C(N)=O)n3)c2)C1=O]

### Step 1: Synthesis of 3-amino-6-(5-bromo-2-fluorophenyl)pyridine-2-carboxamide

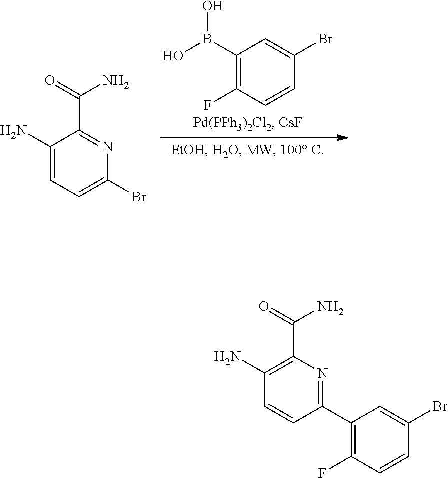 [SMILES: NC(=O)c1nc(Br)ccc1N ; OB(O)c1cc(Br)ccc1F ; NC(=O)c1nc(-c2cc(Br)ccc2F)ccc1N]

Similar to as described in General Procedure X, 3-amino-6-bromopyridine-2-carboxamide was reacted with (5-bromo-2-fluorophenyl)boronic acid to give the title compound (152 mg, 71%) as a white solid. LC-MS (ES, m/z): 310 [M+H]⁺.

### Step 2: Synthesis of 3-amino-6-(2-fluoro-5-[2-[(3R)-3-hydroxy-1-methyl-2-oxopyrrolidin-3-yl]ethynyl]phenyl)pyridine-2-carboxamide

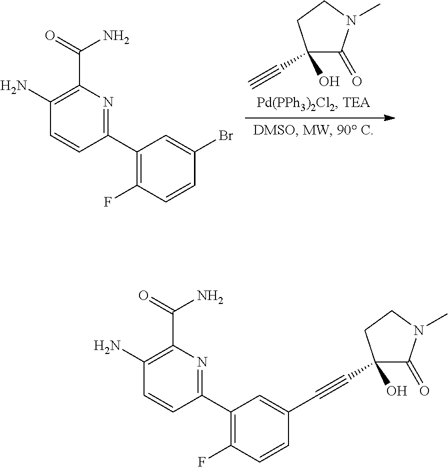

Similar to as described in General Procedure G, 3-amino-6-(5-bromo-2-fluorophenyl)pyridine-2-carboxamide was reacted with (3R)-3-ethynyl-3-hydroxy-1-methylpyrrolidin-2-one to give the title compound (24 mg, 16%) as a white solid. LC-MS (ES, m/z): 369 [M+H]⁺. ¹H NMR (300 MHz, CD₃OD) δ 8.12-8.09 (m, 1H), 7.74-7.43 (m, 1H), 7.26-7.16 (m, 2H), 3.51-3.42 (m, 2H), 2.95 (s, 3H), 2.65-2.57 (m, 1H), 2.37-2.31 (m, 1H).

### Example EEEE

### Synthesis of (R)-6-(2-fluoro-5-((3-hydroxy-1-methyl-2-oxopyrrolidin-3-yl)ethynyl)phenyl)-3-(oxetan-3-ylamino)picolinamide

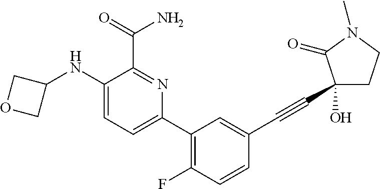 [SMILES: CN1CC[C@@](O)(C#Cc2ccc(F)c(-c3ccc(NC4COC4)c(C(N)=O)n3)c2)C1=O]

### Step 1: Synthesis of ethyl 3-fluoropyridine-2-carboxylate

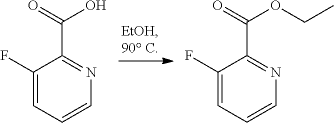

A solution of 3-fluoropyridine-2-carboxylic acid (1.4 g, 9.92 mmol, 1.00 equiv) and sulfuric acid (0.5 mL, 9.38 mmol, 0.20 equiv) in ethanol (20 mL) was stirred at 90° C. for 10 h. The resulting mixture was cooled to room temperature and concentrated under vacuum. The residue was diluted with ethyl acetate and washed with brine. The residue was applied onto a silica gel column with ethyl acetate/petroleum ether (1:20) to give the title compound (1.5 g, 89%) as a colorless solid. LC-MS (ES, m/z): 170 [M+H]⁺.

### Step 2: Synthesis of ethyl 3-[(oxetan-3-yl)amino]pyridine-2-carboxylate

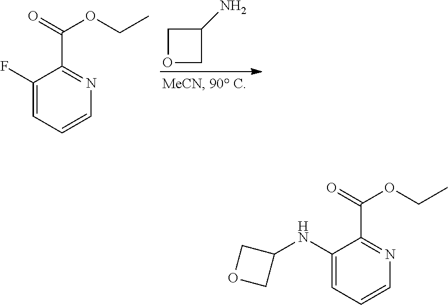

A solution of ethyl 3-fluoropyridine-2-carboxylate (600 mg, 3.55 mmol, 1.00 equiv), triethylamine (900 mg, 8.89 mmol, 2.50 equiv), and oxetan-3-amine (1.32 g, 18.06 mmol, 5.00 equiv) in DMSO (18 mL) was irradiated with microwave for 1.5 h at 150° C. The reaction was cooled to room temperature and concentrated under vacuum. The resulting solution was diluted with 20 mL of ethyl acetate and washed with brine. The residue was applied onto a silica gel column with ethyl acetate/petroleum ether (1:3) to give the title compound (0.39 g, 49%) as yellow oil. LC-MS (ES, m/z): 223 [M+H]⁺.

### Step 3: Synthesis of ethyl 6-bromo-3-[(oxetan-3-yl)amino]pyridine-2-carboxylate

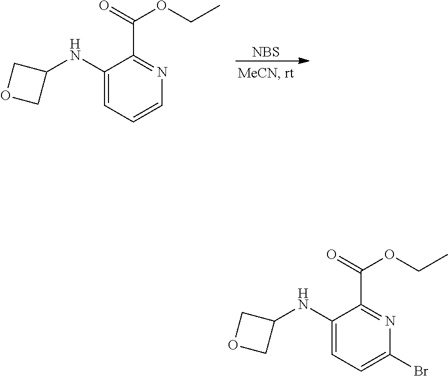

A solution of ethyl 3-[(oxetan-3-yl)amino]pyridine-2-carboxylate (390 mg, 1.75 mmol, 1.00 equiv) and NBS (370 mg, 2.08 mmol, 1.20 equiv) in acetonitrile (20 mL) was stirred for 12 h at room temperature. The resulting mixture was concentrated under vacuum and the residue was applied onto a silica gel column with ethyl acetate/petroleum ether (1:10) to give the title compound (0.4 g, 76%) as a white solid LC-MS (ES, m/z): 301 [M+H]⁺.

### Step 4: Synthesis of 6-bromo-3-[(oxetan-3-yl)amino]pyridine-2-carboxamide

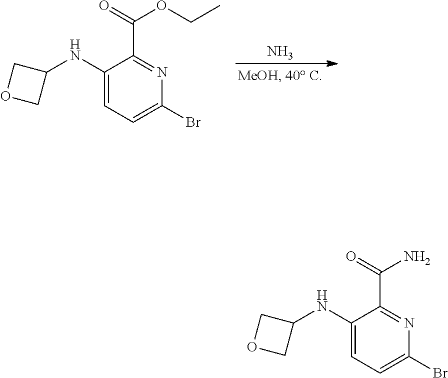

Similar to as described in General Procedure S, ethyl 6-bromo-3-[(oxetan-3-yl)amino]pyridine-2-carboxylate was reacted with ammonia to give the title compound (0.45 g, 94%) as a yellow solid. LC-MS (ES, m/z): 272 [M+H]⁺.

### Step 5: Synthesis of 6-(5-bromo-2-fluorophenyl)-3-[(oxetan-3-yl)amino]pyridine-2-carboxamide

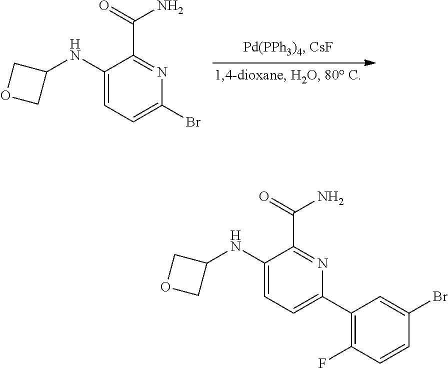 [SMILES: NC(=O)c1nc(Br)ccc1NC1COC1 ; NC(=O)c1nc(-c2cc(Br)ccc2F)ccc1NC1COC1]

Similar to as described in General Procedure X, 6-bromo-3-[(oxetan-3-yl)amino]pyridine-2-carboxamide was reacted with (5-bromo-2-fluorophenyl)boronic acid to give the title compound (0.109 g, 40%) as a yellow solid. LC-MS (ES, m/z): 366 [M+H]⁺.

### Step 6: Synthesis of 6-(2-fluoro-5-[2-[(3R)-3-hydroxy-1-methyl-2-oxopyrrolidin-3-yl]ethynyl]phenyl)-3-[(oxetan-3-yl)amino]pyridine-2-carboxamide

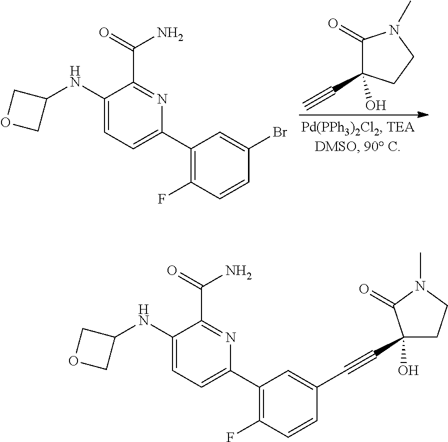

Similar to as described in General Procedure G, 6-(5-bromo-2-fluorophenyl)-3-[(oxetan-3-yl)amino]pyridine-2-carboxamide was reacted with (3R)-3-ethynyl-3-hydroxy-1-methylpyrrolidin-2-one to give the title compound (0.027 g, 20%) as a yellow solid. LC-MS (ES, m/z): 425 [M+H]⁺. ¹H NMR (DMSO-d₆) δ 9.00 (d, J=3 Hz, 1H), 8.23 (s, 1H), 8.11-8.07 (m, 1H), 7.78-7.74 (m, 1H), 7.65 (s, 1H), 7.46-7.41 (m, 1H), 7.35-7.28 (m, 1H), 7.06 (d, J=4.5 Hz, 1H), 6.44 (s, 1H), 4.92 (t, J=6.6 Hz, 2H), 4.77-4.70 (m, 1H), 4.44 (t, J=6 Hz, 2H), 3.35 (t, J=6 Hz, 1H), 2.79 (s, 3H), 2.51-2.41 (m, 1H), 2.22-2.15 (m, 1H).

### Example FFFF

### Synthesis of (R)-6-(2-fluoro-5-((3-hydroxy-1-methyl-2-oxopyrrolidin-3-yl)ethynyl)phenyl)-3-((2-methoxyethyl)amino)picolinamide

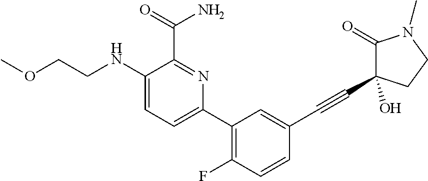 [SMILES: COCCNc1ccc(-c2cc(C#C[C@]3(O)CCN(C)C3=O)ccc2F)nc1C(N)=O]

### Step 1: Synthesis of ethyl 3-[(2-methoxyethyl)amino]pyridine-2-carboxylate

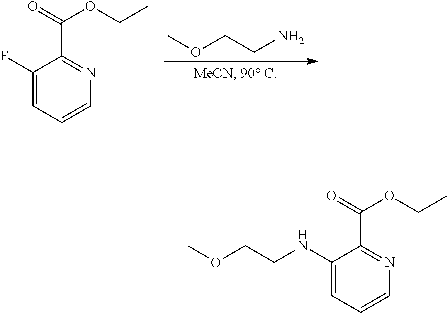

To a solution of 2-methoxyethan-1-amine (600 mg, 7.99 mmol, 10.00 equiv) and triethylamine (200 mg, 1.98 mmol, 2.50 equiv) in acetonitrile (16 mL) was added 3-fluoropyridine-2-carboxylate (140 mg, 0.83 mmol, 1.00 equiv) and the reaction was heated to 90° C. for 24 hours. The resulting mixture was cooled to room temperature and concentrated under vacuum. The residue was applied onto a silica gel column with ethyl acetate/petroleum ether (1:20) to give the title compound (0.16 g, 86%) as orange oil. LC-MS (ES, m/z): 225 [M+H]⁺.

### Step 2: Synthesis of ethyl 6-bromo-3-[(2-methoxyethyl)amino]pyridine-2-carboxylate

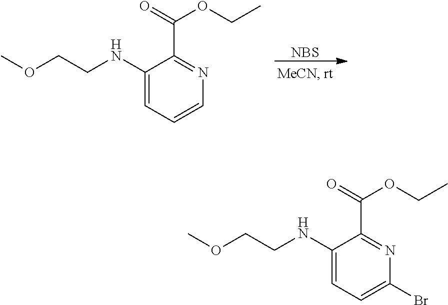

A solution of ethyl 3-[(2-methoxyethyl)amino]pyridine-2-carboxylate (1.4 g, 6.24 mmol, 1.00 equiv) and NBS (1.32 g, 7.42 mmol, 1.20 equiv) in acetonitrile (50 mL) was stirred for 1.5 hours at room temperature. The resulting mixture was concentrated under vacuum and the residue was applied onto a silica gel column with ethyl acetate/petroleum ether (1:10). This resulted in 1.8 g (95%) of the title compound as orange oil. LC-MS (ES, m/z): 303 [M+H]⁺.

### Step 3: Synthesis of 6-bromo-3-[(2-methoxyethyl)amino]pyridine-2-carboxamide

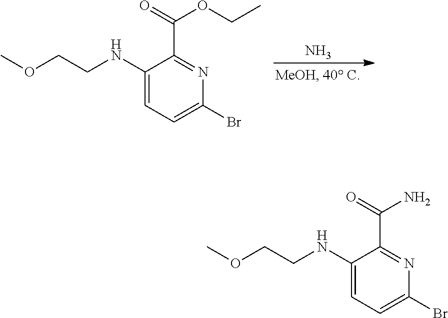

Similar to as described in General Procedure S, ethyl 6-bromo-3-[(2-methoxyethyl)amino]pyridine-2-carboxylate was reacted with ammonia to give the title compound (0.8 g, 88%) as a yellow solid. LC-MS (ES, m/z): 274 [M+H]⁺.

### Step 4: Synthesis of 6-(5-bromo-2-fluorophenyl)-3-[(2-methoxyethyl)amino]pyridine-2-carboxamide

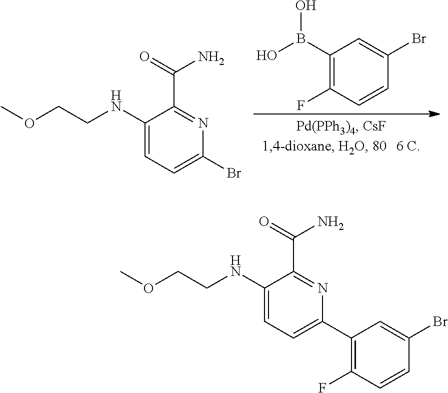 [SMILES: COCCNc1ccc(Br)nc1C(N)=O ; OB(O)c1cc(Br)ccc1F ; COCCNc1ccc(-c2cc(Br)ccc2F)nc1C(N)=O]

Similar to as described in General Procedure X, 6-bromo-3-[(2-methoxyethyl)amino]pyridine-2-carboxamide was reacted with (5-bromo-2-fluorophenyl)boronic acid to give the title compound (0.5 g, 75%) as a yellow solid. LC-MS (ES, m/z): 368 [M+H]⁺.

### Step 5: Synthesis of 6-(2-fluoro-5-[2-[(3R)-3-hydroxy-1-methyl-2-oxopyrrolidin-3-yl]ethynyl]phenyl)-3-[(2-methoxyethyl)amino]pyridine-2-carboxamide

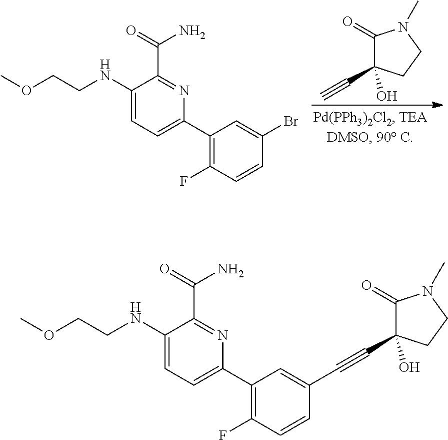

Similar to as described in General Procedure G, 6-(5-bromo-2-fluorophenyl)-3-[(2-methoxyethyl)amino]pyridine-2-carboxamide was reacted with (3R)-3-ethynyl-3-hydroxy-1-methylpyrrolidin-2-one to give the title compound (0.003 g, 1.3%) as a yellow solid. LC-MS (ES, m/z): 427 [M+H]⁺. ¹H NMR (300 MHz, DMSO-d₆) δ 8.66 (t, J=5.6 Hz, 1H), 8.13-8.07 (m, 1H), 7.78-7.74 (m, 1H), 7.51 (s, 1H), 7.45-7.39 (m, 1H), 7.39-7.31 (m, 1H), 6.45 (s, 1H), 3.55 (t, J=5.6 Hz, 1H), 3.41-3.03 (m, 7H), 2.79 (s, 3H), 2.51-2.41 (m, 1H), 2.27-2.13 (m, 1H).

### Example GGGG

### Synthesis of (R)-5-(cyclopropylamino)-2-(3-((3-hydroxy-1-methyl-2-oxopyrrolidin-3-yl)ethynyl)phenyl)pyrimidine-4-carboxamide

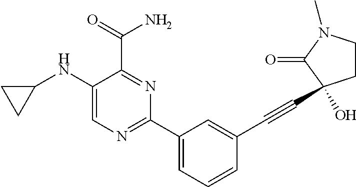 [SMILES: CN1CC[C@@](O)(C#Cc2cccc(-c3ncc(NC4CC4)c(C(N)=O)n3)c2)C1=O]

### Step 1: Synthesis of ethyl 2-chloro-5-(cyclopropylamino)pyrimidine-4-carboxylate

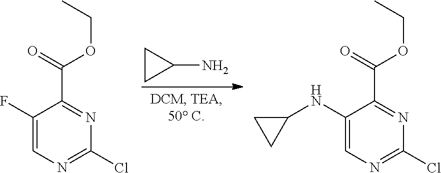

Similar to as described in General Procedure A, ethyl 2-chloro-5-fluoropyrimidine-4-carboxylate was reacted with cyclopropylamine to give the title compound (320 mg, 54%) as a yellow solid. LC-MS (ES, m/z): 242 [M+H]⁺.

### Step 2: Synthesis of 2-chloro-5-(cyclopropylamino)pyrimidine-4-carboxamide

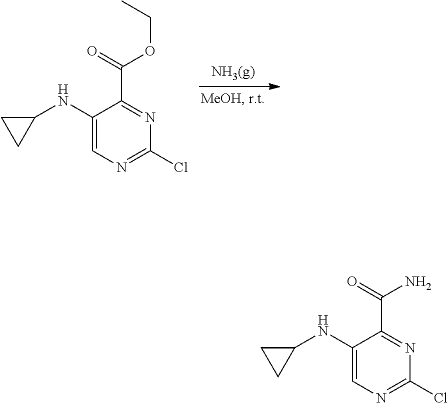 [SMILES: CCOC(=O)c1nc(Cl)ncc1NC1CC1 ; NC(=O)c1nc(Cl)ncc1NC1CC1]

Similar to as described in General Procedure S, ethyl 2-chloro-5-(cyclopropylamino)pyrimidine-4-carboxylate was reacted with ammonia to give the title compound (190 mg, 70%) as a yellow solid. LC-MS (ES, m/z): 213 [M+H]⁺.

### Step 3: Synthesis of 2-(3-[2-[(3R)-3-hydroxy-1-methyl-2-oxopyrrolidin-3-yl]ethynyl]phenyl)-5-[(2-methoxyethyl)amino]pyrimidine-4-carboxamide

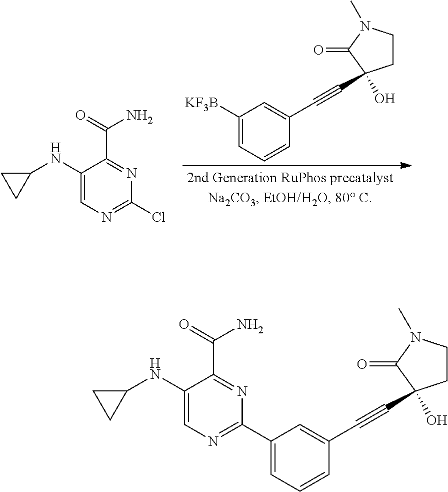

Similar to as described in General Procedure U, 2-chloro-5-(cyclopropylamino)pyrimidine-4-carboxamide was reacted with potassium trifluoro(3-2-[(3R)-3-hydroxy-1-methyl-2-oxopyrrolidin-3-yl]ethynylphenyl)boranideto give the title compound (52.8 mg, 14%) as a yellow solid. LC-MS (ES, m/z): 433 [M+H]⁺. ¹H NMR (300 MHz, DMSO-d₆) δ 8.87 (s, 1H), 8.60 (s, 1H), 8.50-8.44 (m, 2H), 8.28 (s, 1H), 7.86 (s, 1H), 7.50-7.45 (m, 2H), 6.48 (s, 1H), 3.36 (m, 3H), 2.81 (s, 3H), 2.65 (s, 1H), 2.50 (m, 1H), 2.21-2.15 (m, 1H), 0.87-0.85 (m, 2H), 0.57-0.55 (m, 2H).

### Example HHHH

### Synthesis of (R)-2-(3-((3-hydroxy-1-methyl-2-oxopyrrolidin-3-yl)ethynyl)phenyl)-5-(oxetan-3-ylamino)pyrimidine-4-carboxamide

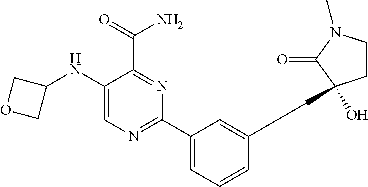 [SMILES: CN1CC[C@@](O)(CCc2cccc(-c3ncc(NC4COC4)c(C(N)=O)n3)c2)C1=O]

### Step 1: Synthesis of ethyl 2-chloro-5-[(oxetan-3-yl)amino]pyrimidine-4-carboxylate

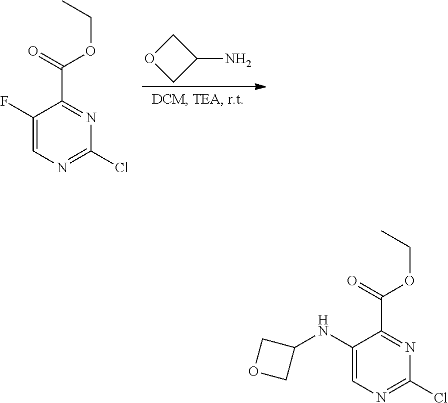 [SMILES: CCOC(=O)c1nc(Cl)ncc1F ; NC1COC1 ; CCOC(=O)c1nc(Cl)ncc1NC1COC1]

Similar to as described in General Procedure A, ethyl 2-chloro-5-fluoropyrimidine-4-carboxylate was reacted with oxetan-3-amine to give the title compound (120 mg, 24%) as a yellow solid. LC-MS (ES, m/z): 258 [M+H]⁺.

### Step 2: Synthesis of 2-chloro-5-[(oxetan-3-yl)amino]pyrimidine-4-carboxamide

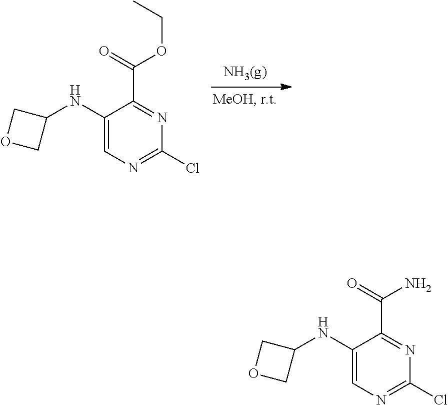 [SMILES: CCOC(=O)c1nc(Cl)ncc1NC1COC1 ; NC(=O)c1nc(Cl)ncc1NC1COC1]

Similar to as described in General Procedure S, ethyl 2-chloro-5-[(oxetan-3-yl)amino]pyrimidine-4-carboxylate was reacted with ammonia to give the title compound (100 mg, 94%) as a yellow solid.

### Step 3: Synthesis of 2-(3-[2-[(3R)-3-hydroxy-1-methyl-2-oxopyrrolidin-3-yl]ethynyl]phenyl)-5-[(oxetan-3-yl)amino]pyrimidine-4-carboxamide

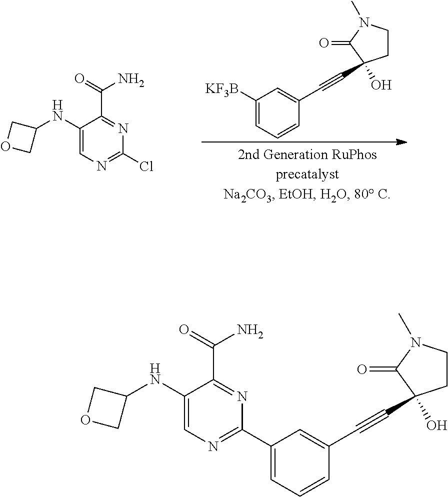

Similar to as described in General Procedure U, 2-chloro-5-[(oxetan-3-yl)amino]pyrimidine-4-carboxamide was reacted with potassium trifluoro(3-2-[(3R)-3-hydroxy-1-methyl-2-oxopyrrolidin-3-yl]ethynylphenyl)boranideto give the title compound (24.2 mg, 15%) as a yellow solid. LC-MS (ES, m/z): 408 [M+H]⁺. ¹H NMR (300 MHz, CD₃OD) δ 8.46 (s, 1H), 8.38 (d, J=4.8 Hz, 1H), 8.35 (s, 1H), 7.50-7.41 (m, 2H), 5.12-5.06 (m, 2H), 4.96-4.90 (m, 2H), 4.65-4.61 (m, 2H), 3.50-3.45 (m, 2H), 2.94 (s, 3H), 2.65-2.57 (m, 1H), 2.37-2.28 (m, 1H).

### Example IIII

### Synthesis of

(R)-2-(3-((3-hydroxy-1-methyl-2-oxopyrrolidin-3-yl)ethynyl)phenyl)-5-((2-methoxyethyl)amino)pyrimidine-4-carboxamide

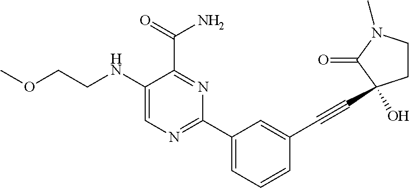 [SMILES: COCCNc1cnc(-c2cccc(C#C[C@]3(O)CCN(C)C3=O)c2)nc1C(N)=O]

### Step 1: Synthesis of ethyl 2-chloro-5-[(2-methoxyethyl)amino]pyrimidine-4-carboxylate

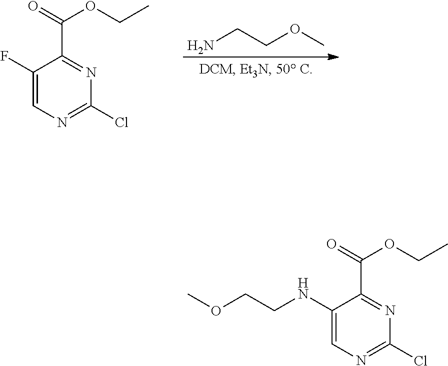

Similar to as described in General Procedure A, ethyl 2-chloro-5-fluoropyrimidine-4-carboxylate was reacted with 2-methoxyethan-1-amine to give the title compound (240 mg, 38%) as a yellow solid. LC-MS (ES, m/z): 260 [M+H]⁺.

### Step 2: Synthesis of 2-chloro-5-[(2-methoxyethyl)amino]pyrimidine-4-carboxamide

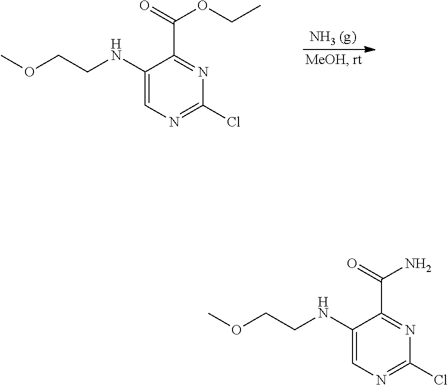 [SMILES: CCOC(=O)c1nc(Cl)ncc1NCCOC ; COCCNc1cnc(Cl)nc1C(N)=O]

Similar to as described in General Procedure S, ethyl 2-chloro-5-[(2-methoxyethyl)amino]pyrimidine-4-carboxylate was reacted with ammonia to give the title compound (220 mg, crude) as a yellow solid. LC-MS (ES, m/z): 231 [M+H]⁺.

### Step 3: Synthesis of 2-(3-[2-[(3R)-3-hydroxy-1-methyl-2-oxopyrrolidin-3-yl]ethynyl]phenyl)-5-[(2-methoxyethyl)amino]pyrimidine-4-carboxamide

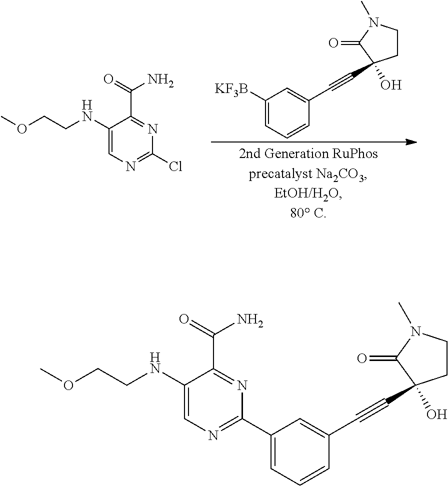

Similar to as described in General Procedure U, 2-chloro-5-[(2-methoxyethyl)amino]pyrimidine-4-carboxamide was reacted with potassium trifluoro(3-2-[(3R)-3-hydroxy-1-methyl-2-oxopyrrolidin-3-yl]ethynylphenyl)boranideto give the title compound (52.8 mg, 14%) as a yellow solid. LC-MS (ES, m/z): 410 [M+H]⁺. ¹H NMR (300 MHz, CD₃OD) δ 8.62 (s, 1H), 8.44 (s, 1H), 8.36 (d, J=7.2 Hz, 1H), 7.49-7.41 (m, 2H), 3.70-3.65 (m, 2H), 3.56-3.49 (m, 4H), 3.42 (s, 3H), 2.94 (s, 3H), 2.65-2.57 (m, 1H), 2.37-2.28 (m, 1H).

### Example JJJJ

### Synthesis of (R)-6-(3-((3-hydroxy-1-methyl-2-oxopyrrolidin-3-yl)ethynyl)phenyl)-4-methoxypicolinamide

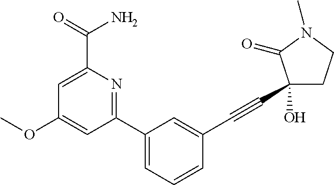 [SMILES: COc1cc(C(N)=O)nc(-c2cccc(C#C[C@]3(O)CCN(C)C3=O)c2)c1]

### Step 1: Synthesis of (3R)-3-[2-[3-(6-bromo-4-methoxypyridin-2-yl)phenyl]ethynyl]-3-hydroxy-1-methylpyrrolidin-2-one

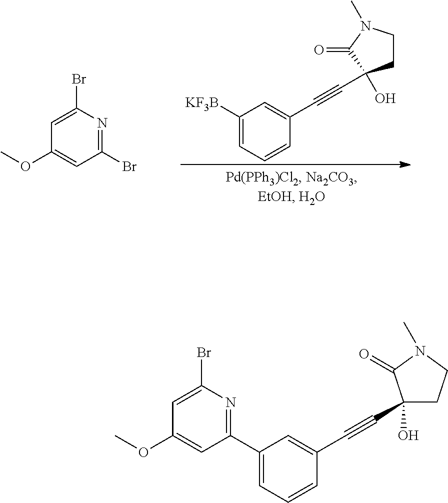

Similar to as described in General Procedure U, 2,6-dibromo-4-methoxypyridine was reacted with potassium trifluoro(3-2-[(3R)-3-hydroxy-1-methyl-2-oxopyrrolidin-3-yl]ethynylphenyl)boranideto give the title compound (180 mg (54%) as a yellow solid. LC-MS (ES, m/z): 401 [M+H]⁺.

### Step 2: Synthesis of methyl 6-(3-[2-[(3R)-3-hydroxy-1-methyl-2-oxopyrrolidin-3-yl]ethynyl]phenyl)-4-methoxypyridine-2-carboxylate

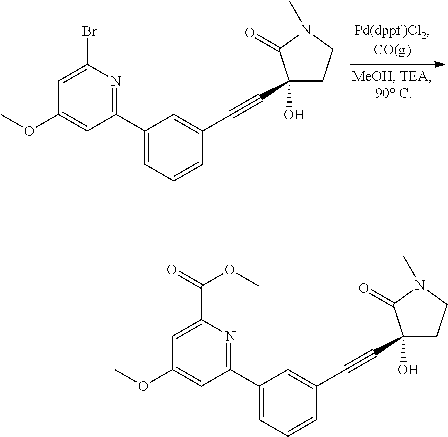 [SMILES: COc1cc(Br)nc(-c2cccc(C#C[C@]3(O)CCN(C)C3=O)c2)c1 ; COC(=O)c1cc(OC)cc(-c2cccc(C#C[C@]3(O)CCN(C)C3=O)c2)n1]

Similar to as described in General Procedure O, (3R)-3-[2-[3-(6-bromo-4-methoxypyridin-2-yl)phenyl]ethynyl]-3-hydroxy-1-methylpyrrolidin-2-one was reacted with carbon monoxide to give the title compound (130 mg, 76%) as a reddish solid. LC-MS (ES, m/z): 381 [M+H]⁺.

### Step 3: Synthesis of 6-(3-[2-[(3R)-3-hydroxy-1-methyl-2-oxopyrrolidin-3-yl]ethynyl]phenyl)-4-methoxypyridine-2-carboxamide

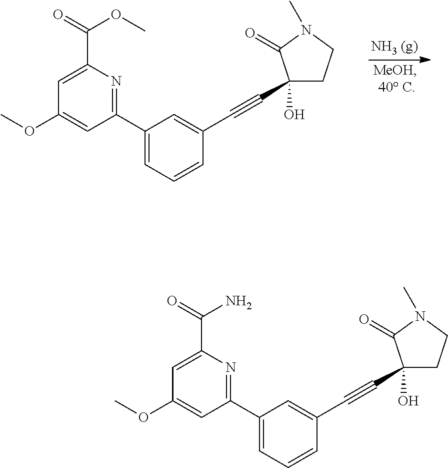 [SMILES: COC(=O)c1cc(OC)cc(-c2cccc(C#C[C@]3(O)CCN(C)C3=O)c2)n1 ; COc1cc(C(N)=O)nc(-c2cccc(C#C[C@]3(O)CCN(C)C3=O)c2)c1]

Similar to as described in General Procedure S, 6-(3-[2-[(3R)-3-hydroxy-1-methyl-2-oxopyrrolidin-3-yl]ethynyl]phenyl)-4-methoxypyridine-2-carboxylate was reacted with ammonia to give the title compound (35.1 mg, 28.11%) as a yellow solid. LC-MS (ES, m/z): 366 [M+H]⁺. ¹H NMR (400 MHz, DMSO-d₆) δ 8.31 (s, 1H), 8.30 (s, 1H), 7.68 (s, 1H), 7.57-7.56 (m, 3H), 3.98 (s, 3H), 3.41 (t, 2H), 2.83 (s, 3H), 2.52-2.50 (m, 1H), 2.30-2.21 (m, 1H).

### Example KKKK

### Synthesis of (R)-2-(2-fluoro-3-((3-hydroxy-1-methyl-2-oxopyrrolidin-3-yl)ethynyl)phenyl)pyrimidine-4-carboxamide

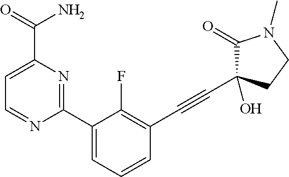 [SMILES: CN1CC[C@@](O)(C#Cc2cccc(-c3nccc(C(N)=O)n3)c2F)C1=O]

### Step 1: Synthesis of 2-chloropyrimidine-4-carboxamide

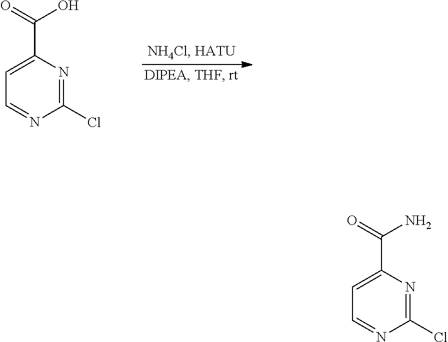 [SMILES: O=C(O)c1ccnc(Cl)n1 ; NC(=O)c1ccnc(Cl)n1]

Similar to as described in General Procedure B, 2-chloropyrimidine-4-carboxylic acid was reacted with ammonium chloride to afford the title compound (1.1 g, 69%) as a yellow solid. LC-MS (ES, m/z): 158 [M+H]⁺.

### Step 2: Synthesis of 2-(3-bromo-2-fluorophenyl)pyrimidine-4-carboxamide

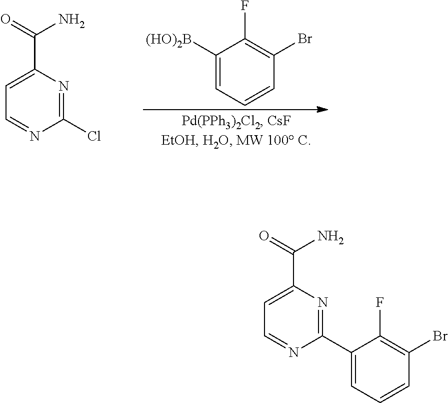 [SMILES: NC(=O)c1ccnc(Cl)n1 ; OB(O)c1cccc(Br)c1F ; NC(=O)c1ccnc(-c2cccc(Br)c2F)n1]

Similar to as described in General Procedure X, 2-chloropyrimidine-4-carboxamide was reacted with (3-bromo-2-fluorophenyl)boronic acid to afford the title compound (450 mg, 48%) as a yellow solid. LC-MS (ES, m/z): 296 [M+H]⁺.

### Step 3: Synthesis of 2-(2-fluoro-3-[2-[(3R)-3-hydroxy-1-methyl-2-oxopyrrolidin-3-yl]ethynyl]phenyl)pyrimidine-4-carboxamide

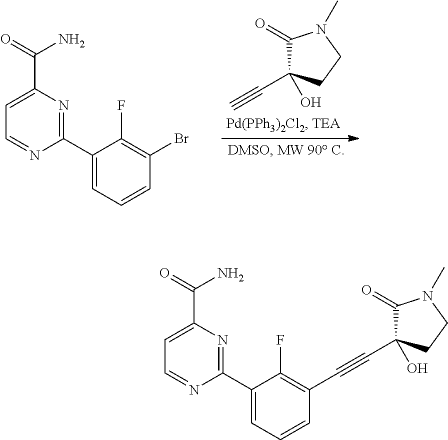

Similar to as described in General Procedure G, 2-(3-bromo-2-fluorophenyl)pyrimidine-4-carboxamide was reacted with (3R)-3-ethynyl-3-hydroxy-1-methylpyrrolidin-2-one to give the title compound (120.3 mg, 22%) as an off-white solid. LC-MS (ES, m/z): 355 [M+H]⁺. ¹H NMR (400 MHz, CD₃OD) δ 9.15 (d, J=4.8 Hz, 1H), 8.21-8.18 (m, 1H), 8.05 (d, J=4.8 Hz, 1H), 7.69-7.66 (m, 1H), 7.36-7.32 (m, 1H), 3.55-3.47 (m, 2H), 2.95 (s, 3H), 2.66-2.60 (m, 4H), 2.38-2.32 (m, 1H).

### Example LLLL

### Synthesis of (R)-6-(2-fluoro-5-((3-hydroxy-1-methyl-2-oxopyrrolidin-3-yl)ethynyl)phenyl)picolinamide

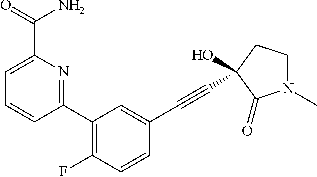 [SMILES: CN1CC[C@@](O)(C#Cc2ccc(F)c(-c3cccc(C(N)=O)n3)c2)C1=O]

### Step 1: Synthesis of ethyl 6-(5-bromo-2-fluorophenyl)pyridine-2-carboxylate

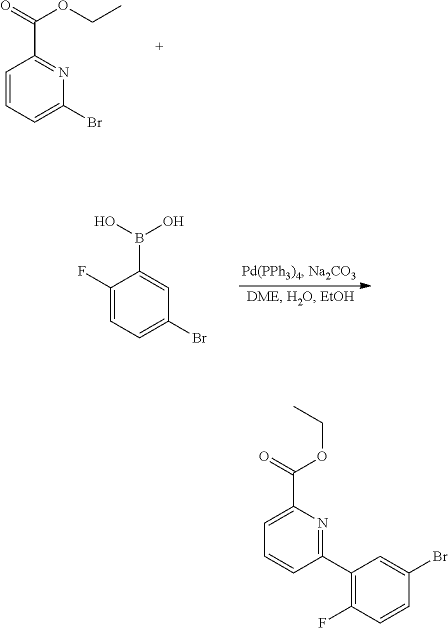 [SMILES: CCOC(=O)c1cccc(Br)n1 ; OB(O)c1cc(Br)ccc1F ; CCOC(=O)c1cccc(-c2cc(Br)ccc2F)n1]

Similar to as described in General Procedure M, ethyl 6-bromopyridine-2-carboxylate was reacted with (5-bromo-2-fluorophenyl)boronic acid to give the title compound (200 mg, 62%) as a yellow solid. LC-MS (ES, m/z): 324, 326 [M+H]⁺.

### Step 2: Synthesis of ethyl 6-(2-fluoro-5-[2-[(3R)-3-hydroxy-1-methyl-2-oxopyrrolidin-3-yl]ethynyl]phenyl)pyridine-2-carboxylate

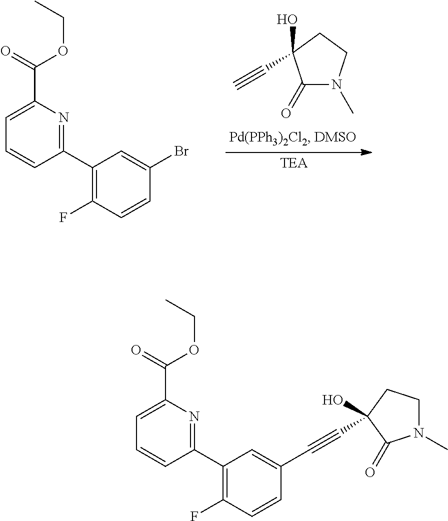

Similar to as described in General Procedure G, ethyl 6-(5-bromo-2-fluorophenyl)pyridine-2-carboxylate was reacted with (3R)-3-ethynyl-3-hydroxy-1-methylpyrrolidin-2-one to give the title compound (200 mg, 85%) as a yellow solid. LC-MS (ES, m/z): 383 [M+H]⁺.

### Step 3: Synthesis of 6-(2-fluoro-5-[2-[(3R)-3-hydroxy-1-methyl-2-oxopyrrolidin-3-yl]ethynyl]phenyl)pyridine-2-carboxamide

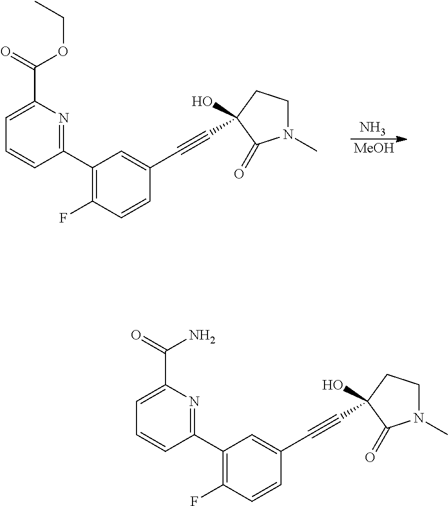

Similar to as described in General Procedure S, ethyl 6-(2-fluoro-5-[2-[(3R)-3-hydroxy-1-methyl-2-oxopyrrolidin-3-yl]ethynyl]phenyl)pyridine-2-carboxylate was reacted with ammonia to give the title compound (55.7 mg, 30%) as a light yellow solid. LC-MS (ES, m/z): 354 [M+H]⁺. ¹H NMR (400 MHz, CD3OD) δ 8.33 (s, 1H), 8.23-8021 (m, 1H), 8.13-8.12 (m, 1H), 8.10-8.05 (m, 1H), 8.01-7.99 (m, 1H), 7.72 (s, 1H), 7.61-7.57 (m, 1H), 7.44-7.39 (m, 1H), 3.38-3.35 (m, 2H), 2.81 (s, 3H), 2.49-2.44 (m, 1H), 2.23-2.17 (s, 1H).

### Example MMMM

### Synthesis of (R)-2-(3-((3-hydroxy-1-methyl-2-oxopyrrolidin-3-yl)ethynyl)phenyl)-6-methoxyquinazoline-4-carboxamide

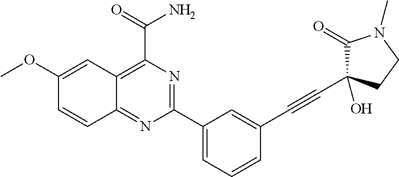 [SMILES: COc1ccc2nc(-c3cccc(C#C[C@]4(O)CCN(C)C4=O)c3)nc(C(N)=O)c2c1]

### Step 1: Synthesis of 2-chloro-4-(1-ethoxyvinyl)-6-methoxyquinazoline

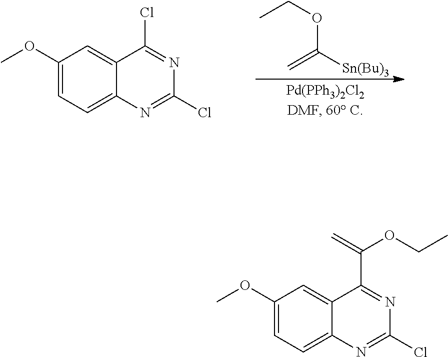

Similar to as described in General Procedure Q, 2,4-dichloro-6-methoxyquinazoline was reacted with tributyl(1-ethoxyethenyl)stannane to afford the title compound (550 mg, 48%) as a yellow solid. LC-MS (ES, m/z): 265 [M+H]⁺.

### Step 2: Synthesis of ethyl 2-chloro-6-methoxyquinazoline-4-carboxylate

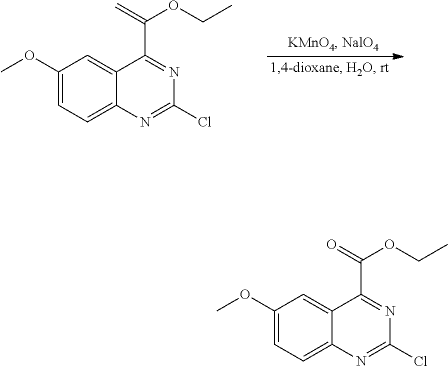 [SMILES: C=C(OCC)c1nc(Cl)nc2ccc(OC)cc12 ; CCOC(=O)c1nc(Cl)nc2ccc(OC)cc12]

Similar to as described in General Procedure R, 2-chloro-4-(1-ethoxyethenyl)-6-methoxyquinazoline was reacted with potassium permanganate to afford the title compound (260 mg, 26%) as a yellow solid. LC-MS (ES, m/z): 267 [M+H]⁺.

### Step 3: Synthesis of ethyl 2-(3-bromophenyl)-6-methoxyquinazoline-4-carboxylate

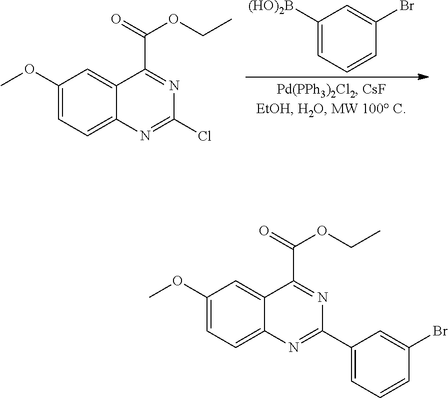 [SMILES: CCOC(=O)c1nc(Cl)nc2ccc(OC)cc12 ; OB(O)c1cccc(Br)c1 ; CCOC(=O)c1nc(-c2cccc(Br)c2)nc2ccc(OC)cc12]

Similar to as described in General Procedure X, ethyl 2-chloro-6-methoxyquinazoline-4-carboxylate was reacted with (3-bromophenyl)boronic acid to afford the title compound (160 mg, 42%) as a yellow solid. LC-MS (ES, m/z): 387 [M+H]⁺.

### Step 4: Synthesis of (R)-ethyl 2-(3-((3-hydroxy-1-methyl-2-oxopyrrolidin-3-yl)ethynyl)phenyl)-6-methoxyquinazoline-4-carboxylate

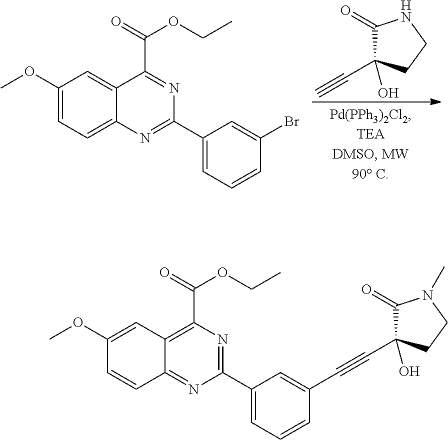

Similar to as described in General Procedure G, ethyl 2-(3-bromophenyl)-6-methoxyquinazoline-4-carboxylate was reacted with (3R)-3-ethynyl-3-hydroxy-1-methylpyrrolidin-2-one to afford the title compound (130 mg, 71%) as yellow oil. LC-MS (ES, m/z): 446 [M+H]⁺.

### Step 5: Synthesis of (R)-2-(3-((3-hydroxy-1-methyl-2-oxopyrrolidin-3-yl)ethynyl)phenyl)-6-methoxyquinazoline-4-carboxamide

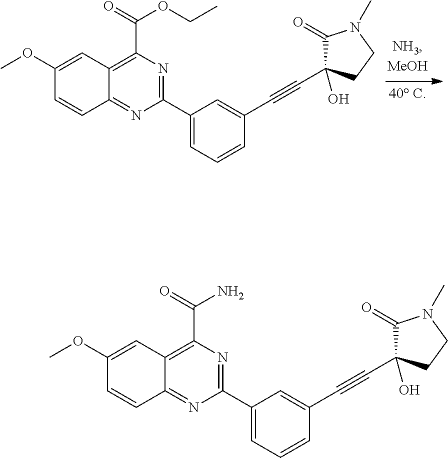 [SMILES: CCOC(=O)c1nc(-c2cccc(C#C[C@]3(O)CCN(C)C3=O)c2)nc2ccc(OC)cc12 ; COc1ccc2nc(-c3cccc(C#C[C@]4(O)CCN(C)C4=O)c3)nc(C(N)=O)c2c1]

Similar to as described in General Procedure S, ethyl 2-(3-[2-[(3R)-3-hydroxy-1-methyl-2-oxopyrrolidin-3-yl]ethynyl]phenyl)-6-methoxyquinazoline-4-carboxylate was reacted with ammonia to give the title compound (67.1 mg, 55%) as a white solid. LC-MS (ES, m/z): 417 [M+H]⁺. ¹H NMR (400 MHz, DMSO-d₆) δ 8.68-8.65 (m, 3H), 8.29-8.26 (m, 1H), 8.11-8.09 (m, 2H), 7.76-7.73 (m, 1H), 7.60-7.59 (m, 2H), 6.53 (s, 1H), 3.94 (s, 3H), 3.39-3.37 (m, 2H), 2.83 (s, 3H), 2.47-2.44 (m, 1H), 2.25-2.18 (m, 1H).

### Example NNNN

### Synthesis of (R)-2-(3-((3-hydroxy-1-methyl-2-oxopyrrolidin-3-yl)ethynyl)-5-methylphenyl)pyrimidine-4-carboxamide

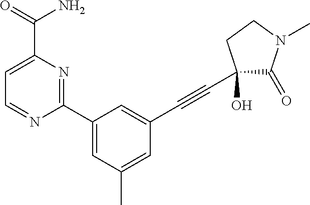 [SMILES: Cc1cc(C#C[C@]2(O)CCN(C)C2=O)cc(-c2nccc(C(N)=O)n2)c1]

### Step 1: Synthesis of 2-chloropyrimidine-4-carboxamide

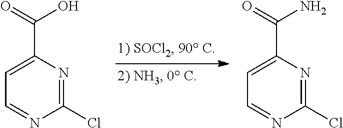 [SMILES: O=C(O)c1ccnc(Cl)n1 ; NC(=O)c1ccnc(Cl)n1]

A solution of 2-chloropyrimidine-4-carboxylic acid (500 mg, 1.00 equiv) in thionyl chloride (10 mL) was heated to 90° C. for 30 minutes and then was concentrated under vacuum. The reaction was treated with 20 mL of ammonium hydroxide at 0° C. The resulting solution was extracted with dichloromethane, dried over anhydrous sodium sulfate, and concentrated under vacuum. This resulted in 320 mg of the title compound as a white solid. LC-MS (ES, m/z): 158 [M+H]⁺.

### Step 2: Synthesis of 2-(3-bromo-5-methylphenyl)pyrimidine-4-carboxamide

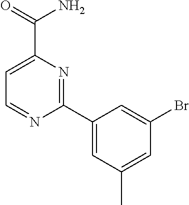 [SMILES: Cc1cc(Br)cc(-c2nccc(C(N)=O)n2)c1]

Similar to as described in General Procedure M, 2-chloropyrimidine-4-carboxamide was reacted with (3-bromo-5-methylphenyl)boronic acid to give the title compound (300 mg, 54%) as a white solid. LC-MS (ES, m/z): 292, 294 [M+H]⁺.

### Step 3: Synthesis of (R)-2-(3-((3-hydroxy-1-methyl-2-oxopyrrolidin-3-yl)ethynyl)-5-methylphenyl)pyrimidine-4-carboxamide

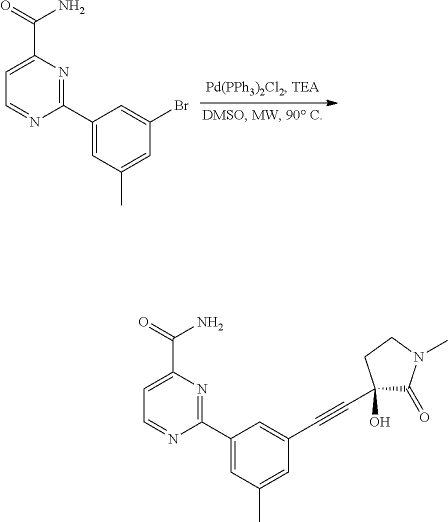 [SMILES: Cc1cc(Br)cc(-c2nccc(C(N)=O)n2)c1 ; Cc1cc(C#C[C@]2(O)CCN(C)C2=O)cc(-c2nccc(C(N)=O)n2)c1]

Similar to as described in General Procedure G, 2-(3-bromo-5-methylphenyl)pyrimidine-4-carboxamide was reacted with (3R)-3-ethynyl-3-hydroxy-1-methylpyrrolidin-2-one to give the title compound (54.6 mg) as an off-white solid. LC-MS (ES, m/z): 351 [M+H]⁺. ¹H NMR (400 MHz, CD₃OD) δ 9.10 (d, J=5.2 Hz, 1H), 8.49 (s, 1H), 8.45 (s, 1H), 7.99 (d, J=5.2 Hz, 1H), 7.48 (s, 1H), 3.53-3.49 (m, 2H), 2.96 (s, 3H), 2.66-2.57 (m, 1H), 2.50 (s, 3H), 2.36-2.33 (m, 1H).

### Example OOOO

### Synthesis of (R)-2-(3-((3-hydroxy-1-methyl-2-oxopyrrolidin-3-yl)ethynyl)phenyl)-6-(6-methylpyridin-3-yl)pyrimidine-4-carboxamide

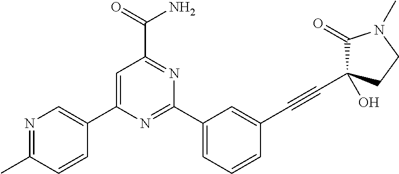 [SMILES: Cc1ccc(-c2cc(C(N)=O)nc(-c3cccc(C#C[C@]4(O)CCN(C)C4=O)c3)n2)cn1]

### Step 1: Synthesis of (R)-ethyl 2-(3-((3-hydroxy-1-methyl-2-oxopyrrolidin-3-yl)ethynyl)phenyl)-6-(6-methylpyridin-3-yl)pyrimidine-4-carboxylate

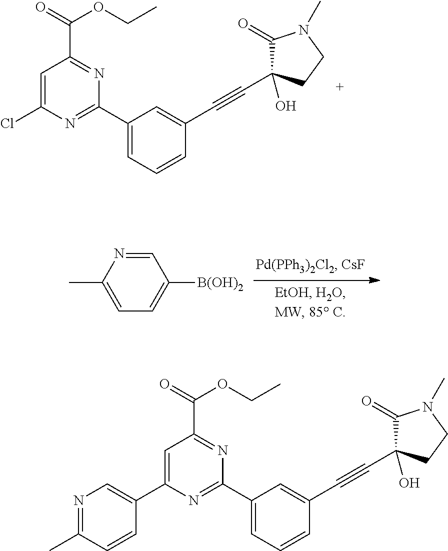

Similar to as described in General Procedure X, ethyl 6-chloro-2-(3-[2-[(3R)-3-hydroxy-1-methyl-2-oxopyrrolidin-3-yl]ethynyl]phenyl)pyrimidine-4-carboxylate was reacted with (6-methylpyridin-3-yl)boronic acid to give the title compound (156 mg, 46%) as a yellow solid. LC-MS (ES, m/z): 457 [M+H]⁺.

### Step 2: Synthesis of (R)-2-(3-((3-hydroxy-1-methyl-2-oxopyrrolidin-3-yl)ethynyl)phenyl)-6-(6-methylpyridin-3-yl)pyrimidine-4-carboxamide

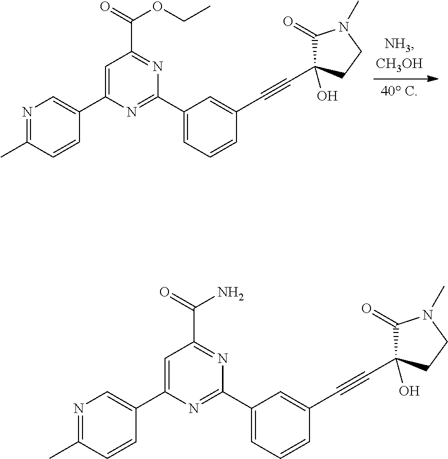 [SMILES: CCOC(=O)c1cc(-c2ccc(C)nc2)nc(-c2cccc(C#C[C@]3(O)CCN(C)C3=O)c2)n1 ; Cc1ccc(-c2cc(C(N)=O)nc(-c3cccc(C#C[C@]4(O)CCN(C)C4=O)c3)n2)cn1]

Similar to as described in General Procedure S, ethyl 2-(3-[2-[(3R)-3-hydroxy-1-methyl-2-oxopyrrolidin-3-yl]ethynyl]phenyl)-6-(6-methylpyridin-3-yl)pyrimidine-4-carboxylate was reacted with ammonia to give the title compound (56.4 mg, 43%) as a white solid. LC-MS (ES, m/z): 428 [M+H]⁺. ¹H NMR (400 MHz, CD₃OD) δ 9.38 (s, 1H), 8.78 (s, 1H), 8.71-8.65 (m, 2H), 8.50 (s, 1H), 7.66 (d, J=8.0 Hz, 1H), 7.57 (t, J=8.0 Hz, 2H), 3.58-3.48 (m, 2H), 2.97 (s, 3H), 2.66-2.63 (m, 1H), 2.40-2.33 (m, 1H).

### Example PPPP

### Synthesis of (R)-2-(3-((3-hydroxy-1-methyl-2-oxopyrrolidin-3-yl)ethynyl)phenyl)-6-(pyrazin-2-yl)pyrimidine-4-carboxamide

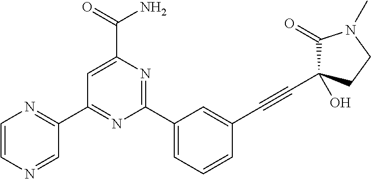 [SMILES: CN1CC[C@@](O)(C#Cc2cccc(-c3nc(C(N)=O)cc(-c4cnccn4)n3)c2)C1=O]

### Step 1: Synthesis of (R)-ethyl 2-(3-((3-hydroxy-1-methyl-2-oxopyrrolidin-3-yl)ethynyl)phenyl)-6-(pyrazin-2-yl)pyrimidine-4-carboxylate

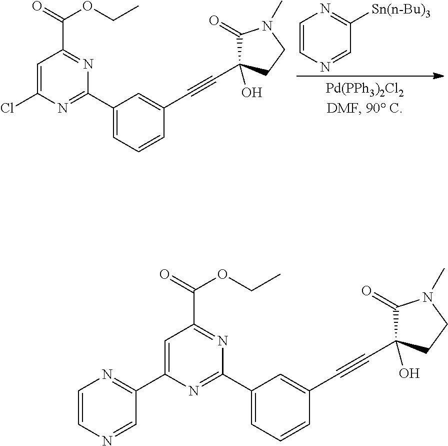

A suspension of ethyl 6-chloro-2-(3-[2-[(3R)-3-hydroxy-1-methyl-2-oxopyrrolidin-3-yl]ethynyl]phenyl)pyrimidine-4-carboxylate (200 mg, 0.50 mmol, 1.00 equiv), 2-(tributylstannyl)pyrazine (550 mg, 1.49 mmol, 3.00 equiv), and Pd(PPh₃)₂Cl₂ (40 mg, 0.06 mmol, 0.10 equiv) in DMF (10 mL) was stirred for 2 h at 80° C. under nitrogen. After cooling the reaction was quenched by saturated potassium fluoride solution and diluted with ethyl acetate. The precipitate was filtered off and the filtrate was washed with water and brine. The organic layer was dried over anhydrous sodium sulfate and concentrated under vacuum. The residue was purified silica gel column chromatography eluting with dichloromethane/methanol (10:1) to afford the title compound (130 mg, 59%) as a red solid. LC-MS (ES, m/z): 444 [M+H]⁺.

### Step 2: Synthesis of (R)-2-(3-((3-hydroxy-1-methyl-2-oxopyrrolidin-3-yl)ethynyl)phenyl)-6-(pyrazin-2-yl)pyrimidine-4-carboxamide

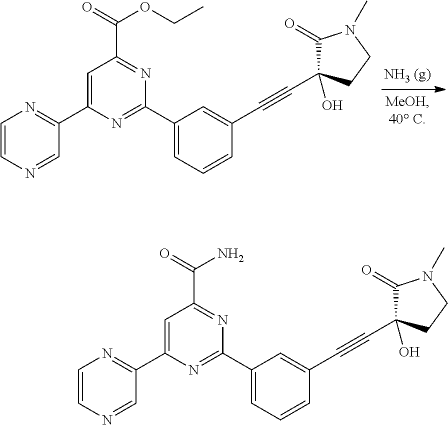 [SMILES: CCOC(=O)c1cc(-c2cnccn2)nc(-c2cccc(C#C[C@]3(O)CCN(C)C3=O)c2)n1 ; CN1CC[C@@](O)(C#Cc2cccc(-c3nc(C(N)=O)cc(-c4cnccn4)n3)c2)C1=O]

Similar to as described in General Procedure S, ethyl 2-(3-[2-[(3R)-3-hydroxy-1-methyl-2-oxopyrrolidin-3-yl]ethynyl]phenyl)-6-(pyrazin-2-yl)pyrimidine-4-carboxylate was treated with ammonia to give the title compound (54.7 mg, 45%) as a white solid. LC-MS (ES, m/z): 415 [M+H]⁺. ¹H NMR (400 Mhz, DMSO-d₆) δ 9.87 (s, 1H), 8.92 (s, 1H), 8.86-8.80 (m, 4H), 8.75 (s, 1H), 8.10 (s, 1H), 7.67-7.60 (m, 2H), 6.53 (s, 1H), 3.40-3.37 (m, 2H), 2.82 (s, 3H), 2.50-2.45 (m, 1H), 2.24-2.19 (m, 1H).

### Example QQQQ

### Synthesis of (R)-6-(3,5-difluorophenyl)-2-(3-((3-hydroxy-1-methyl-2-oxopyrrolidin-3-yl)ethynyl)phenyl)pyrimidine-4-carboxamide

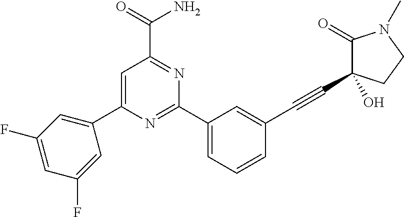 [SMILES: CN1CC[C@@](O)(C#Cc2cccc(-c3nc(C(N)=O)cc(-c4cc(F)cc(F)c4)n3)c2)C1=O]

### Step 1: Synthesis of (R)-ethyl 6-chloro-2-(3-((3-hydroxy-1-methyl-2-oxopyrrolidin-3-yl)ethynyl)phenyl)pyrimidine-4-carboxylate

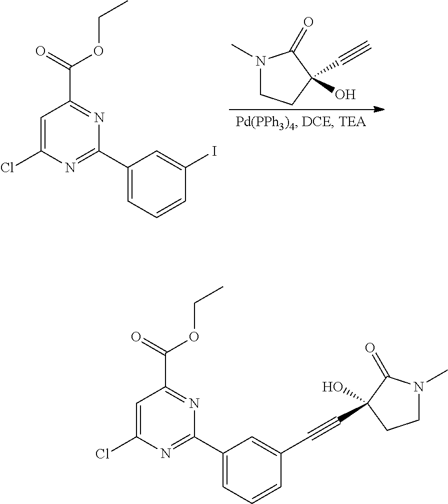

Similar to as described in General Procedure E, ethyl 6-chloro-2-(3-iodophenyl)pyrimidine-4-carboxylate was reacted with (3R)-3-ethynyl-3-hydroxypyrrolidin-2-one to give the title compound (424 mg, 82%) as a light yellow solid. LC-MS (ES, m/z): 400, 402 [M+H]⁺.

### Step 2: Synthesis of (R)-ethyl 6-(3,5-difluorophenyl)-2-(3-((3-hydroxy-1-methyl-2-oxopyrrolidin-3-yl)ethynyl)phenyl)pyrimidine-4-carboxylate

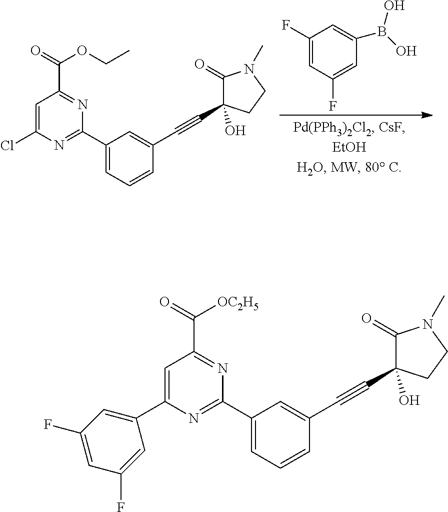

Similar to as described in General Procedure X, ethyl 6-chloro-2-(3-[2-[(3R)-3-hydroxy-1-methyl-2-oxopyrrolidin-3-yl]ethynyl]phenyl)pyrimidine-4-carboxylate was reacted with (3,5-difluorophenyl)boronic acid to give the title compound (84 mg, 47%) as a light brown solid. LC-MS (ES, m/z): 478 [M+H]⁺.

### Step 3: Synthesis of (R)-6-(3,5-difluorophenyl)-2-(3-((3-hydroxy-1-methyl-2-oxopyrrolidin-3-yl)ethynyl)phenyl)pyrimidine-4-carboxamide

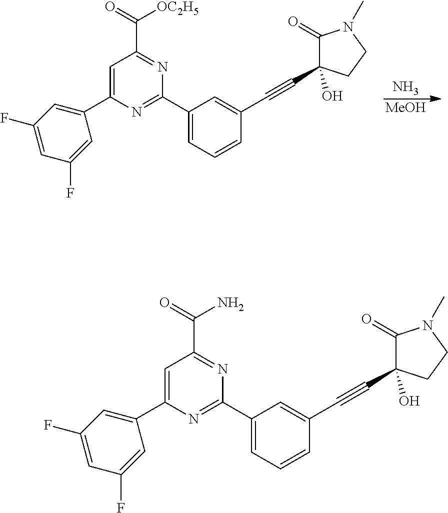

Similar to as described in General Procedure S, ethyl6-(3,5-difluorophenyl)-2-(3-[2-[(3R)-3-hydroxy-1-methyl-2-oxopyrrolidin-3-yl]ethynyl]phenyl)pyrimidine-4-carboxylate was reacted with ammonia to give the title compound (57.5 mg, 38%) as a white solid. LC-MS (ES, m/z): 449 [M+H]⁺. ¹H NMR (400 MHz, CD₃OD) δ 8.79 (s, 1H), 8.73 (d, J=7.6 Hz, 1H), 8.48 (s, 1H), 8.03 (d, J=7.4 Hz, 2H), 7.68 (d, J=7.6 Hz, 1H), 7.69 (t, J=7.6 Hz, 1H), 7.27-7.22 (m, 1H), 3.57-3.48 (m, 2H), 2.97 (s, 3H), 2.68-2.62 (m, 1H), 2.40-2.34 (m, 1H).

### Example RRRR

### Synthesis of (R)-6-(2,5-difluorophenyl)-2-(3-((3-hydroxy-1-methyl-2-oxopyrrolidin-3-yl)ethynyl)phenyl)pyrimidine-4-carboxamide

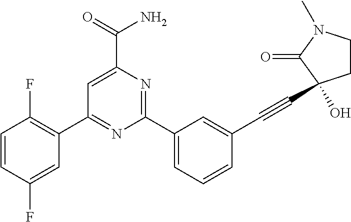 [SMILES: CN1CC[C@@](O)(C#Cc2cccc(-c3nc(C(N)=O)cc(-c4cc(F)ccc4F)n3)c2)C1=O]

### Step 1: Synthesis of

(R)-ethyl6-(2,5-difluorophenyl)-2-(3-((3-hydroxy-1-methyl-2-oxopyrrolidin-3-yl)ethynyl)phenyl)pyrimidine-4-carboxylate

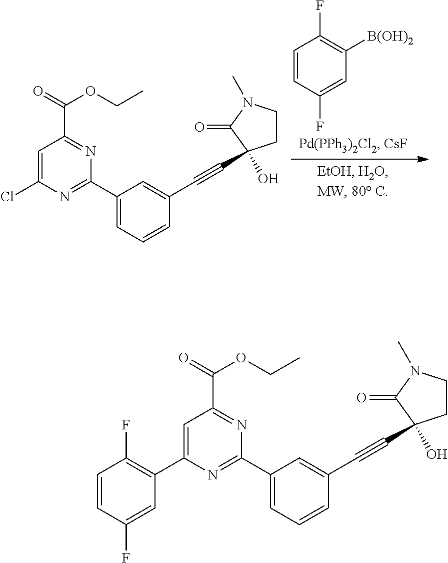

Similar to as described in General Procedure X, ethyl 6-chloro-2-(3-[2-[(3R)-3-hydroxy-1-methyl-2-oxopyrrolidin-3-yl]ethynyl]phenyl)pyrimidine-4-carboxylate was reacted with (2,5-difluorophenyl)boronic acid to give the title compound (125 mg, crude) as a brown solid. LC-MS (ES, m/z): 478 [M+H]⁺.

### Step 2: Synthesis of (R)-6-(2, 5-difluorophenyl)-2-(3-((3-hydroxy-1-methyl-2-oxopyrrolidin-3-yl)ethynyl)phenyl)pyrimidine-4-carboxamide

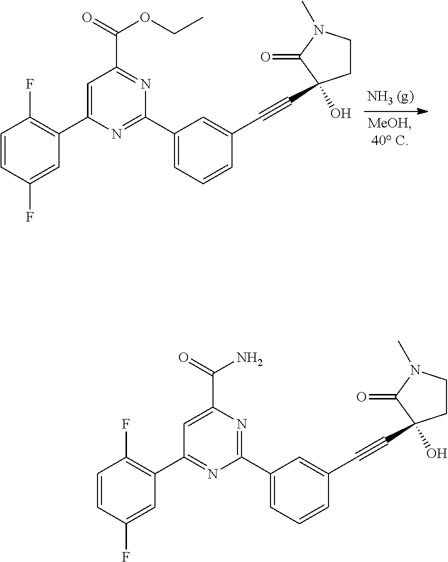 [SMILES: CCOC(=O)c1cc(-c2cc(F)ccc2F)nc(-c2cccc(C#C[C@]3(O)CCN(C)C3=O)c2)n1 ; CN1CC[C@@](O)(C#Cc2cccc(-c3nc(C(N)=O)cc(-c4cc(F)ccc4F)n3)c2)C1=O]

Similar to as described in General Procedure S, ethyl 6-(2,5-difluorophenyl)-2-(3-[2-[(3R)-3-hydroxy-1-methyl-2-oxopyrrolidin-3-yl]ethynyl]phenyl)pyrimidine-4-carboxylate was reacted with ammonia to give the title compound (43.1 mg, 23%) as a white solid. LC-MS (ES, m/z): 449 [M+H]⁺. ¹H NMR (400 MHz, CD₃OD) δ 8.77 (s, 1H), 8.70 (d, J=8.0 Hz, 1H), 8.49 (s, 1H), 8.14-8.11 (m, 1H), 7.67 (d, J=7.6 Hz, 1H), 7.58 (t, J=8.0 Hz, 2H), 3.57-3.48 (m, 2H), 2.96 (s, 3H), 2.68-2.62 (m, 1H), 2.40-2.33 (m, 1H).

### Example SSSS

### Synthesis of (R)-2-(3-((3-hydroxy-1-methyl-2-oxopyrrolidin-3-yl)ethynyl)phenyl)-6-(oxetan-3-yloxy)pyrimidine-4-carboxamide

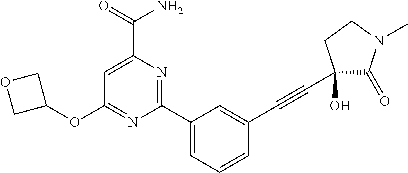 [SMILES: CN1CC[C@@](O)(C#Cc2cccc(-c3nc(OC4COC4)cc(C(N)=O)n3)c2)C1=O]

### Step 1: Synthesis of 2-(3-iodophenyl)-6-(oxetan-3-yloxy)pyrimidine-4-carboxylic acid

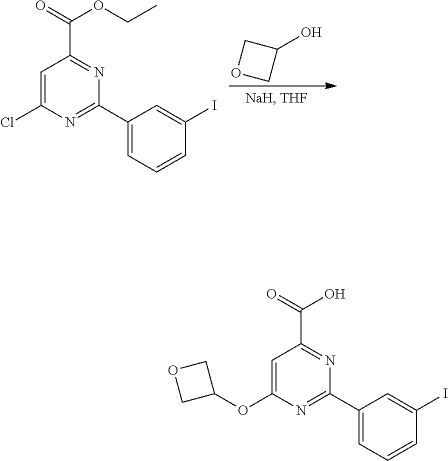

Sodium hydride (150 mg, 6.25 mmol, 3.00 equiv) in THF (50 mL) was added dropwise to a stirred solution of oxetan-3-ol (950 mg, 12.82 mmol, 10.00 equiv) in THF (50 mL) under nitrogen. After a few minutes ethyl 6-chloro-2-(3-iodophenyl)pyrimidine-4-carboxylate (500 mg, 1.29 mmol, 1.00 equiv). was added and the reaction was stirred at room temperature for 2 hours. The resulting mixture was concentrated under vacuum and the residue was purified by a silica gel column chromatography eluting with dichloromethane/methanol (1:20). This resulted in 0.45 g (88%) of the title compound as orange oil. LC-MS (ES, m/z): 399 [M+H]⁺.

### Step 2: Synthesis of 2-(3-iodophenyl)-6-(oxetan-3-yloxy)pyrimidine-4-carboxamide

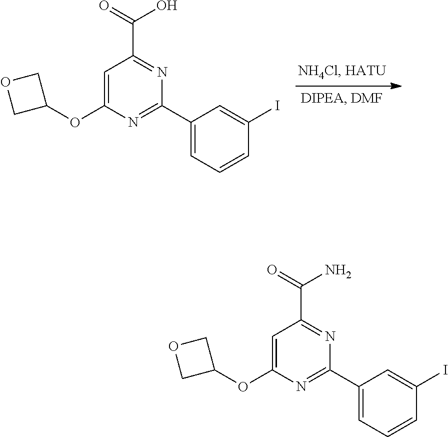 [SMILES: O=C(O)c1cc(OC2COC2)nc(-c2cccc(I)c2)n1 ; NC(=O)c1cc(OC2COC2)nc(-c2cccc(I)c2)n1]

Similar to as described in General Procedure B, 2-(3-iodophenyl)-6-(oxetan-3-yloxy)pyrimidine-4-carboxylic acid was reacted with ammonium chloride to give the title compound (0.3 g, 75%) as a white solid. LC-MS (ES, m/z): 398 [M+H]⁺.

### Step 3: Synthesis of (R)-2-(3-((3-hydroxy-1-methyl-2-oxopyrrolidin-3-yl)ethynyl)phenyl)-6-(oxetan-3-yloxy)pyrimidine-4-carboxamide

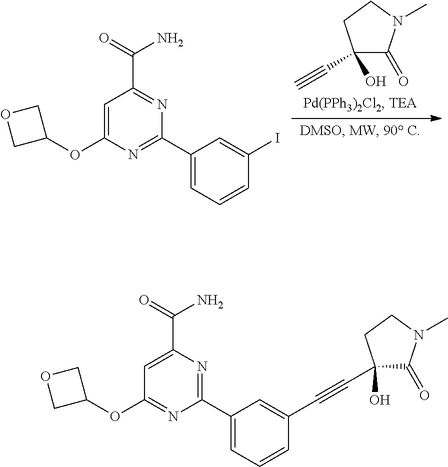

Similar to as described in General Procedure G, 2-(3-iodophenyl)-6-(oxetan-3-yloxy)pyrimidine-4-carboxamide was reacted with (3R)-3-ethynyl-3-hydroxy-1-methylpyrrolidin-2-one to give the title compound (0.0541 g, 35%) as a white solid. LC-MS (ES, m/z): 409 [M+H]⁺. ¹H NMR (CD₃OD) δ 8.61 (s, 1H), 8.56-8.50 (m, 1H), 7.68-7.63 (m, 1H), 7.55-7.51 (m, 1H), 7.47 (s, 1H), 5.95-5.91 (m, 1H), 5.17-5.13 (m, 2H), 4.89-4.82 (m, 2H), 3.53-3.45 (m, 2H), 2.96 (s, 3H), 2.67-2.61 (m, 1H), 2.39-2.34 (m, 1H).

### Example TTTT

### Synthesis of (R)-2-(3-((3-hydroxy-1-methyl-2-oxopyrrolidin-3-yl)ethynyl)phenyl)-5-(oxetan-3-ylmethylamino)pyrimidine-4-carboxamide

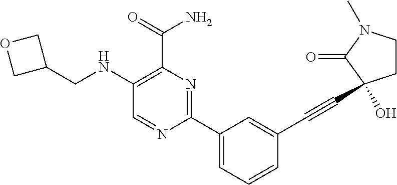 [SMILES: CN1CC[C@@](O)(C#Cc2cccc(-c3ncc(NCC4COC4)c(C(N)=O)n3)c2)C1=O]

### Step 1: Synthesis of ethyl 2-chloro-5-(oxetan-3-ylmethylamino)pyrimidine-4-carboxylate

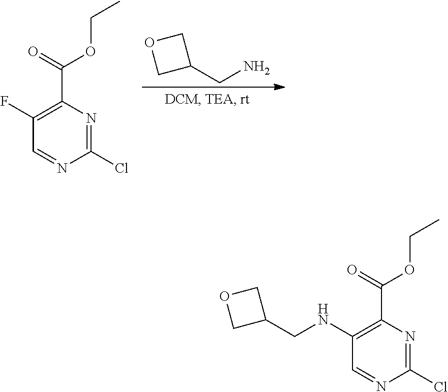 [SMILES: CCOC(=O)c1nc(Cl)ncc1F ; NCC1COC1 ; CCOC(=O)c1nc(Cl)ncc1NCC1COC1]

Similar to as described in General Procedure A, ethyl 2-chloro-5-fluoropyrimidine-4-carboxylate was reacted with oxetan-3-ylmethanamine to give the title compound (100 mg, 25%) as a yellow solid. LC-MS (ES, m/z): 272 [M+H]⁺.

### Step 2: Synthesis of 2-chloro-5-(oxetan-3-ylmethylamino)pyrimidine-4-carboxamide

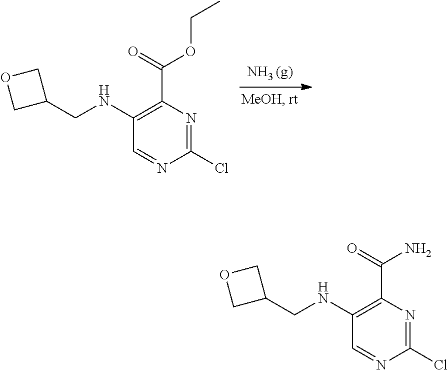 [SMILES: CCOC(=O)c1nc(Cl)ncc1NCC1COC1 ; NC(=O)c1nc(Cl)ncc1NCC1COC1]

Similar to as described in General Procedure S, ethyl 2-chloro-5-[(oxetan-3-ylmethyl)amino]pyrimidine-4-carboxylate was reacted with ammonia to give the title compound (100 mg, 45%) as a yellow solid. LC-MS (ES, m/z): 243 [M+H]⁺.

### Step 3: Synthesis of (R)-2-(3-((3-hydroxy-1-methyl-2-oxopyrrolidin-3-yl)ethynyl)phenyl)-5-(oxetan-3-ylmethylamino)pyrimidine-4-carboxamide

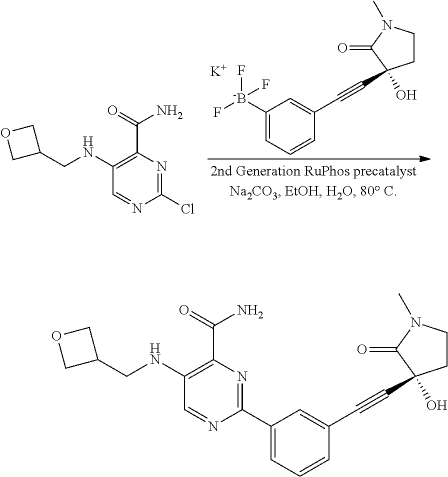

Similar to as described in General Procedure U, 2-chloro-5-[(oxetan-3-ylmethyl)amino]pyrimidine-4-carboxamide was reacted with potassium (R)-trifluoro(3-((3-hydroxy-1-methyl-2-oxopyrrolidin-3-yl)ethynyl)phenyl)borate to give the title compound (31.6 mg, 20%) as a yellow solid. LC-MS (ES, m/z): 422 [M+H]⁺. ¹H NMR (300 MHz, DMSO-d₆) δ 8.67 (s, 1H), 8.58 (s, 1H), 8.48-8.42 (m, 2H), 8.32 (t, J=5.7 Hz, 1H), 7.83 (s, 1H), 7.50-7.42 (m, 2H), 6.47 (s, 1H), 4.69 (dd, J=7.8, 6.0 Hz, 2H), 4.36 (t, J=6.0 Hz, 2H), 3.65 (t, J=6.3 Hz, 2H), 3.38-3.34 (m, 2H), 3.28-3.26 (m, 1H), 2.81 (s, 3H), 2.49-2.42 (m, 1H), 2.23-2.14 (m, 1H).

### Example UUUU

### Synthesis of (R)-2-(3-((3-hydroxy-1-methyl-2-oxopyrrolidin-3-yl)ethynyl)phenyl)-5-(2-methoxy-2-methylpropylamino)pyrimidine-4-carboxamide

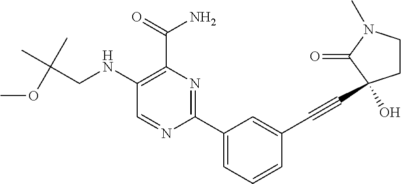 [SMILES: COC(C)(C)CNc1cnc(-c2cccc(C#C[C@]3(O)CCN(C)C3=O)c2)nc1C(N)=O]

### Step 1: Synthesis of ethyl 2-chloro-5-(2-methoxy-2-methylpropylamino)pyrimidine-4-carboxylate

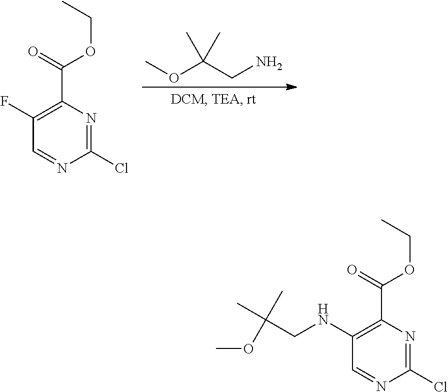

Similar to as described in General Procedure A, ethyl 2-chloro-5-fluoropyrimidine-4-carboxylate was reacted with 2-methoxy-2-methylpropan-1-amine to give the title compound (300 mg, 71%) as a yellow solid. LC-MS (ES, m/z): 288 [M+H]⁺.

### Step 2: Synthesis of 2-chloro-5-(2-methoxy-2-methylpropylamino)pyrimidine-4-carboxamide

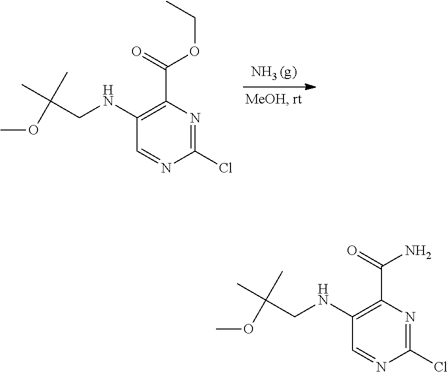 [SMILES: CCOC(=O)c1nc(Cl)ncc1NCC(C)(C)OC ; COC(C)(C)CNc1cnc(Cl)nc1C(N)=O]

Similar to as described in General Procedure S, ethyl 2-chloro-5-[(2-methoxy-2-methylpropyl)amino]pyrimidine-4-carboxylate was reacted with ammonia to give the title compound (250 mg, 93%) as a yellow solid. LC-MS (ES, m/z): 259 [M+H]⁺.

### Step 3: Synthesis of (R)-2-(3-((3-hydroxy-1-methyl-2-oxopyrrolidin-3-yl)ethynyl)phenyl)-5-(2-methoxy-2-methylpropylamino)pyrimidine-4-carboxamide

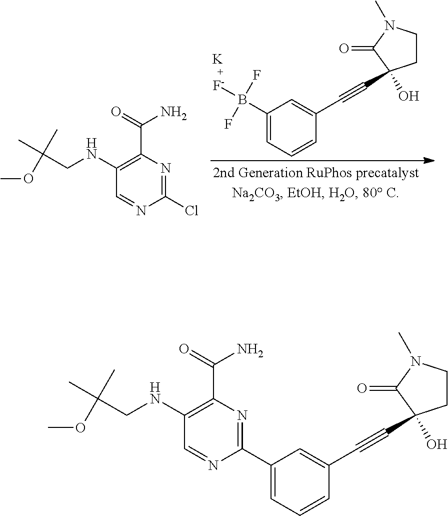

Similar to as described in General Procedure U, ethyl 2-chloro-5-[(2-methoxy-2-methylpropyl)amino]pyrimidine-4-carboxylate was reacted with potassium (R)-trifluoro(3-((3-hydroxy-1-methyl-2-oxopyrrolidin-3-yl)ethynyl)phenyl)borate to give the title compound (60.8 mg, 30%) as a yellow solid. LC-MS (ES, m/z): 438 [M+H]⁺. ¹H NMR (400 MHz, CD₃OD) δ 8.62 (s, 1H), 8.45 (s, 1H), 8.35 (d, J=7.6 Hz, 1H), 7.50-7.43 (m, 2H), 3.54-3.47 (m, 2H), 3.39 (s, 2H), 3.27 (s, 3H), 2.96 (s, 3H), 2.65-2.60 (m, 1H), 2.38-2.31 (m, 1H), 1.31 (s, 6H).

### Example VVVV

### Synthesis of (R)-2-(3-((3-hydroxy-1-methyl-2-oxopyrrolidin-3-yl)ethynyl)phenyl)-5-(2-morpholinoethyl amino)pyrimidine-4-carboxamide

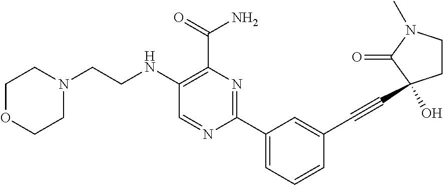 [SMILES: CN1CC[C@@](O)(C#Cc2cccc(-c3ncc(NCCN4CCOCC4)c(C(N)=O)n3)c2)C1=O]

### Step 1: Synthesis of ethyl 2-chloro-5-(2-morpholinoethylamino)pyrimidine-4-carboxylate

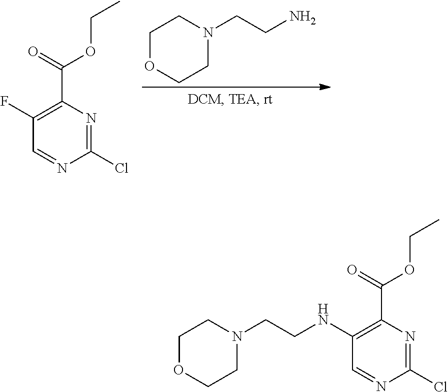

Similar to as described in General Procedure A, ethyl 2-chloro-5-fluoropyrimidine-4-carboxylate was reacted with 2-(morpholin-4-yl)ethan-1-amine to give the title compound (290 mg, 63%) as a yellow solid. LC-MS (ES, m/z): 315 [M+H]⁺.

### Step 2: Synthesis of 2-chloro-5-(2-morpholinoethylamino)pyrimidine-4-carboxamide

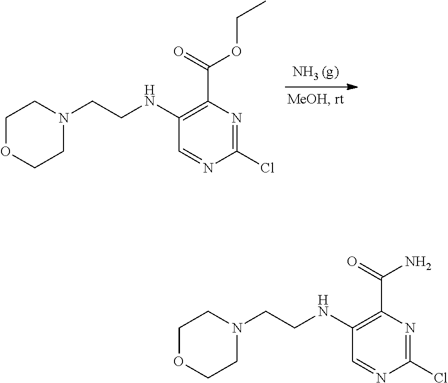 [SMILES: CCOC(=O)c1nc(Cl)ncc1NCCN1CCOCC1 ; NC(=O)c1nc(Cl)ncc1NCCN1CCOCC1]

Similar to as described in General Procedure S, ethyl 2-chloro-5-[[2-(morpholin-4-yl)ethyl]amino]pyrimidine-4-carboxylate was reacted with ammonia to give the title compound (230 mg, 90%) as a yellow solid. LC-MS (ES, m/z): 286 [M+H]⁺.

### Step 3: Synthesis of (R)-2-(3-((3-hydroxy-1-methyl-2-oxopyrrolidin-3-yl)ethynyl)phenyl)-5-(2-morpholinoethylamino)pyrimidine-4-carboxamide

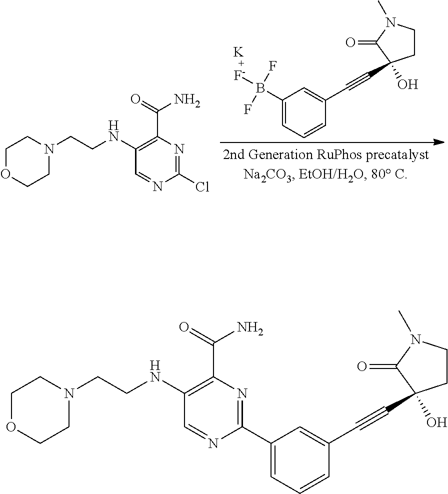

Similar to as described in General Procedure U, 2-chloro-5-[[2-(morpholin-4-yl)ethyl]amino]pyrimidine-4-carboxamide was reacted with potassium (R)-trifluoro(3-((3-hydroxy-1-methyl-2-oxopyrrolidin-3-yl)ethynyl)phenyl)borate to give the title compound (84.7 mg, 24%) as a yellow solid. LC-MS (ES, m/z): 465 [M+H]⁺. ¹H NMR(400 MHz, DMSO-d₆) δ 8.62 (s, 1H), 8.54 (s, 1H), 8.47-8.41 (m, 3H), 7.49-7.42 (m, 2H), 6.47 (s, 1H), 3.60 (t, J=4.4 Hz, 4H), 3.45-3.35 (m, 4H), 2.81 (s, 3H), 2.60 (t, J=6.0 Hz, 1H), 2.50-2.45 (m, 5H), 2.23-2.16 (m, 1H).

### Example WWWW

### Synthesis of (R)-2-(3-((3-hydroxy-1-methyl-2-oxopyrrolidin-3-yl)ethynyl)phenyl)-5-morpholinopyrimidine-4-carboxamide

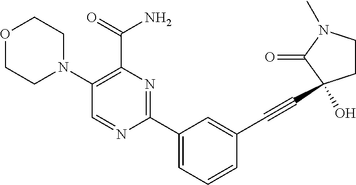 [SMILES: CN1CC[C@@](O)(C#Cc2cccc(-c3ncc(N4CCOCC4)c(C(N)=O)n3)c2)C1=O]

### Step 1: Synthesis of ethyl 2-chloro-5-morpholinopyrimidine-4-carboxylate

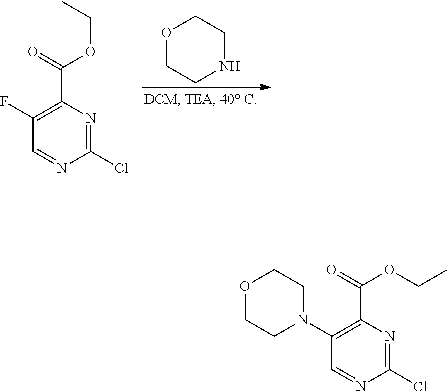 [SMILES: CCOC(=O)c1nc(Cl)ncc1F ; C1COCCN1 ; CCOC(=O)c1nc(Cl)ncc1N1CCOCC1]

Similar to as described in General Procedure A, ethyl 2-chloro-5-fluoropyrimidine-4-carboxylate was reacted morpholine to give the title compound (300 mg, 53%) as a yellow solid. LC-MS (ES, m/z): 272 [M+H]⁺.

### Step 2: Synthesis of 2-chloro-5-morpholinopyrimidine-4-carboxamide

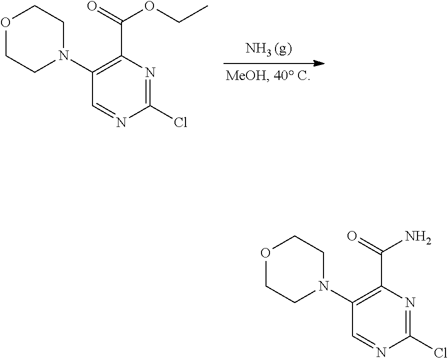 [SMILES: CCOC(=O)c1nc(Cl)ncc1N1CCOCC1 ; NC(=O)c1nc(Cl)ncc1N1CCOCC1]

Similar to as described in General Procedure S, ethyl 2-chloro-5-(morpholin-4-yl)pyrimidine-4-carboxylate was reacted with ammonia to give the title compound (260 mg, 100%) as a yellow solid. LC-MS (ES, m/z): 243 [M+H]⁺.

### Step 3: Synthesis of (R)-2-(3-((3-hydroxy-1-methyl-2-oxopyrrolidin-3-yl)ethynyl)phenyl)-5-morpholinopyrimidine-4-carboxamide

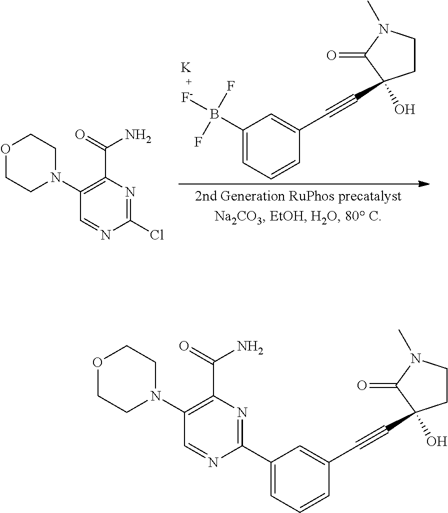

Similar to as described in General Procedure U, 2-chloro-5-(morpholin-4-yl)pyrimidine-4-carboxamide was reacted with potassium (R)-trifluoro(3-((3-hydroxy-1-methyl-2-oxopyrrolidin-3-yl)ethynyl)phenyl)borate to give the title compound (55.1 mg, 12%) as a yellow solid. LC-MS (ES, m/z): 422 [M+H]⁺. ¹H NMR (300 MHz, DMSO-d₆) δ 8.70 (s, 1H), 8.36 (s, 1H), 8.33 (t, J=3.0 Hz, 1H), 8.21 (s, 1H), 7.80 (s, 1H), 7.55-7.49 (m, 2H), 6.49 (s, 1H), 3.73 (t, J=4.5 Hz, 4H), 3.38-3.33 (m, 2H), 3.20 (t, J=4.5 Hz, 4H), 2.81 (s, 3H), 2.49-2.42 (m, 1H), 2.23-2.14 (m, 1H)

### Example XXXX

### Synthesis of (R)-2-(3-((3-hydroxy-1-methyl-2-oxopyrrolidin-3-yl)ethynyl)phenyl)-6-(4-methyl-1H-pyrazol-1-yl)pyrimidine-4-carboxamide

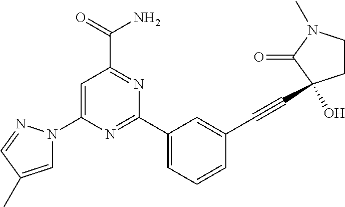 [SMILES: Cc1cnn(-c2cc(C(N)=O)nc(-c3cccc(C#C[C@]4(O)CCN(C)C4=O)c3)n2)c1]

### Step 1: Synthesis of (R)-methyl 2-(3-((3-hydroxy-1-methyl-2-oxopyrrolidin-3-yl)ethynyl)phenyl)-6-(4-methyl-1H-pyrazol-1-yl)pyrimidine-4-carboxylate

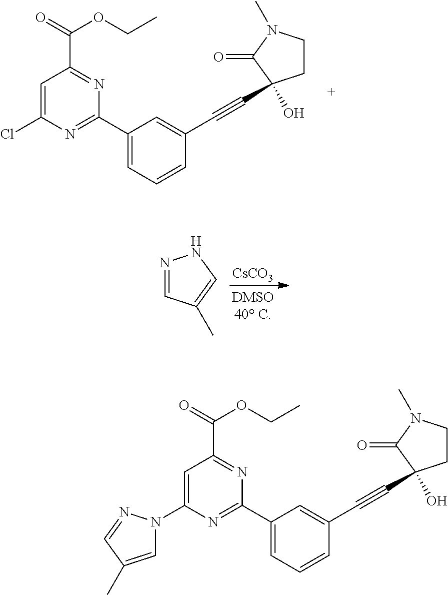

Similar to as described in General Procedure A, ethyl 6-chloro-2-(3-[2-[(3R)-3-hydroxy-1-methyl-2-oxopyrrolidin-3-yl]ethynyl]phenyl)pyrimidine-4-carboxylate was reacted 4-methyl-1H-pyrazole to give the title compound (157.5 mg, 97%) as a light yellow solid. LC-MS (ES, m/z): 432, 434 [M+H]⁺.

### Step 2: Synthesis of (R)-2-(3-((3-hydroxy-1-methyl-2-oxopyrrolidin-3-yl)ethynyl)phenyl)-6-(4-methyl-1H-pyrazol-1-yl)pyrimidine-4-carboxamide

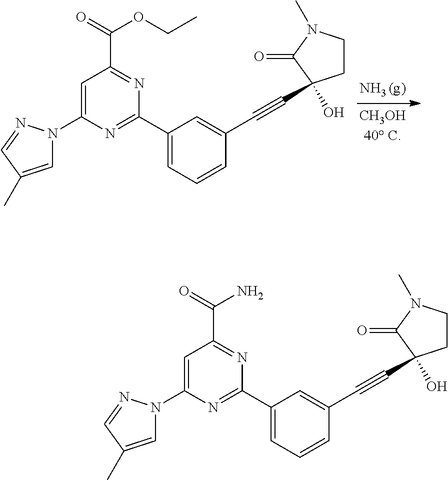 [SMILES: CCOC(=O)c1cc(-n2cc(C)cn2)nc(-c2cccc(C#C[C@]3(O)CCN(C)C3=O)c2)n1 ; Cc1cnn(-c2cc(C(N)=O)nc(-c3cccc(C#C[C@]4(O)CCN(C)C4=O)c3)n2)c1]

Similar to as described in General Procedure S, methyl 2-(3-[2-[(3R)-3-hydroxy-1-methyl-2-oxopyrrolidin-3-yl]ethynyl]phenyl)-6-(4-methyl-1H-pyrazol-1-yl)pyrimidine-4-carboxylate was reacted with ammonia to give the title compound (15.2 mg, 10%) as a light yellow solid. LC-MS (ES, m/z): 417 [M+H]⁺. ¹H NMR (300 MHz, CD₃OD) δ 8.75 (s, 1H), 8.67 (d, J=7.5 Hz, 1H), 8.66 (s, 1H), 8.42 (s, 1H), 7.77 (s, 1H), 7.67 (d, J=7.5 Hz, 1H), 7.57 (t, J=7.8 Hz, 1H), 3.56-3.51 (m, 2H), 2.96 (s, 3H), 2.70-2.62 (m, 1H), 2.39-2.33 (m, 1H), 2.25 (s, 3H).

### Example YYYY

### Synthesis of (R)-2-(3-((3-hydroxy-1-methyl-2-oxopyrrolidin-3-yl)ethynyl)phenyl)-5-((3-methyloxetan-3-yl)methylamino)pyrimidine-4-carboxamide

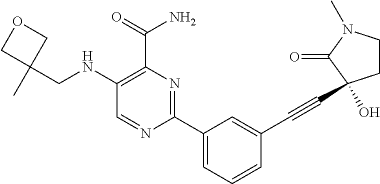 [SMILES: CN1CC[C@@](O)(C#Cc2cccc(-c3ncc(NCC4(C)COC4)c(C(N)=O)n3)c2)C1=O]

### Step 1: Synthesis of ethyl 2-chloro-5-((3-methyloxetan-3-yl)methyl amino)pyrimidine-4-carboxylate

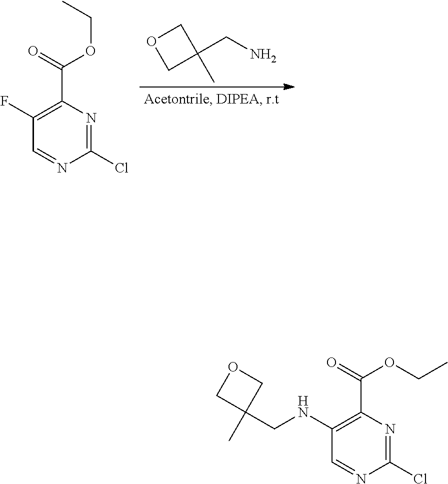

Similar to as described in General Procedure A, ethyl 2-chloro-5-fluoropyrimidine-4-carboxylate was reacted with (3-methyloxetan-3-yl)methylamine to give the title compound (200 mg, 48%) as a yellow solid. LC-MS (ES, m/z): 286 [M+H]⁺.

### Step 2: Synthesis of 2-chloro-5-((3-methyloxetan-3-yl)methylamino)pyrimidine-4-carboxamide

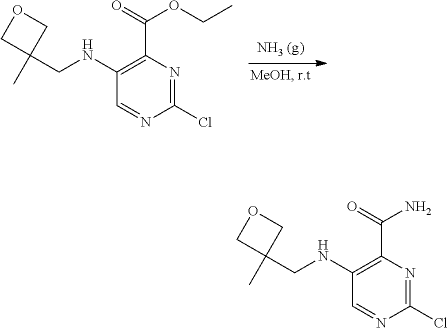 [SMILES: CCOC(=O)c1nc(Cl)ncc1NCC1(C)COC1 ; CC1(CNc2cnc(Cl)nc2C(N)=O)COC1]

Similar to as described in General Procedure S, ethyl 2-chloro-5-[[(3-methyloxetan-3-yl)methyl]amino]pyrimidine-4-carboxylate was reacted with ammonia to give the title compound (170 mg, crude) as a yellow solid. LC-MS (ES, m/z): 257 [M+H]⁺.

### Step 3: Synthesis of (R)-2-(3-((3-hydroxy-1-methyl-2-oxopyrrolidin-3-yl)ethynyl)phenyl)-5-((3-methyloxetan-3-yl)methylamino)pyrimidine-4-carboxamide

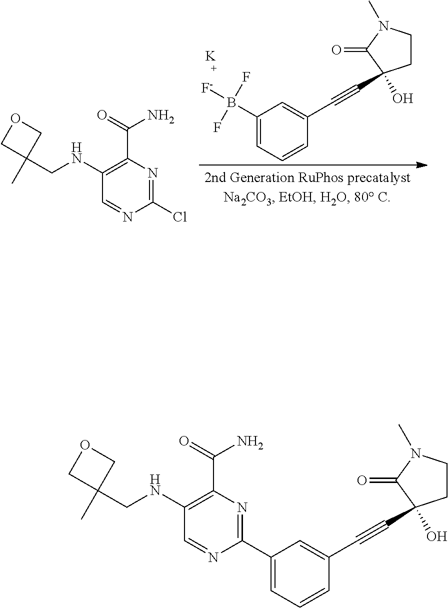

Similar to as described in General Procedure U, 2-chloro-5-[[(3-methyloxetan-3-yl)methyl]amino]pyrimidine-4-carboxamide was reacted with potassium (R)-trifluoro(3-((3-hydroxy-1-methyl-2-oxopyrrolidin-3-yl)ethynyl)phenyl)borate to give the title compound (72 mg, 27%) as a yellow solid. LC-MS (ES, m/z): 436 [M+H]⁺. ¹H NMR (300 MHz, CD₃OD) δ 8.67 (s, 1H), 8.45 (s, 1H), 8.35 (d, J=7.5 Hz, 1H), 7.47-7.43 (m, 2H), 4.59 (d, J=5.7 Hz, 2H), 4.48 (d, J=5.7 Hz, 2H), 3.60 (s, 2H), 3.50 (m, 2H), 2.95 (s, 3H), 2.65-2.59 (m, 1H), 2.37-2.28 (m, 1H), 1.59 (s, 3H).

### Example ZZZZ

### Synthesis of (R)-5-amino-2-(3-((3-hydroxy-1-methyl-2-oxopyrrolidin-3-yl)ethynyl)phenyl)-6-(oxetan-3-yloxy)pyrimidine-4-carboxamide

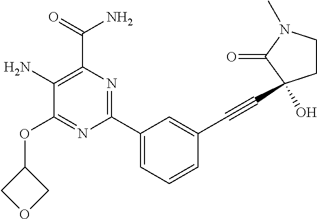 [SMILES: CN1CC[C@@](O)(C#Cc2cccc(-c3nc(OC4COC4)c(N)c(C(N)=O)n3)c2)C1=O]

### Step 1: Synthesis of ethyl 5-amino-2,6-dichloropyrimidine-4-carboxylate

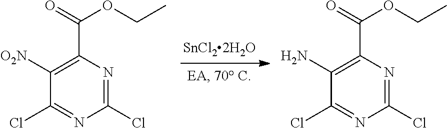 [SMILES: CCOC(=O)c1nc(Cl)nc(Cl)c1[N+](=O)[O-] ; CCOC(=O)c1nc(Cl)nc(Cl)c1N]

A solution of ethyl 2,6-dichloro-5-nitropyrimidine-4-carboxylate (400 mg, 1.5 mmol, 1.00 equiv) and stannous chloride dihydrate (857 mg, 3.8 mmol, 3.0 equiv) in ethyl acetate (15 mL) was stirred for 12 h at 70° C. After completion the reaction was quenched with water and adjusted pH to 8-9 with sodium carbonate solution. The solution was extracted with ethyl acetate, washed with brine, dried over anhydrous sodium sulfate, and concentrated under vacuum. The residue was purified by a silica gel column chromatography eluting with ethyl acetate/petroleum ether (1:5) to give the title compound (460 mg, 32%) as a light yellow solid. LC-MS (ES, m/z): 236 [M+H]⁺.

### Step 2: Synthesis of 5-amino-2-chloro-6-(oxetan-3-yloxy)pyrimidine-4-carboxylate

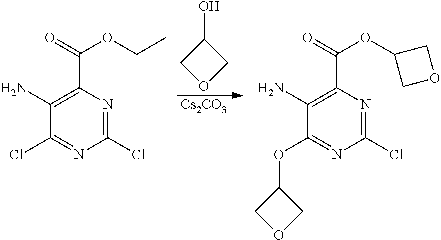

Similar to as described in General Procedure A, ethyl 5-amino-2,6-di chloropyrimidine-4-carboxylate was reacted with oxetan-3-ol to give the title compound (234 mg, 73%) as a light yellow solid. LC-MS (ES, m/z): 302 [M+H]⁺.

### Step 3: Synthesis of ethyl 5-amino-2-(3-[2-[(3R)-3-hydroxy-1-methyl-2-oxopyrrolidin-3-yl]ethynyl]phenyl)-6-(oxetan-3-yloxy)pyrimidine-4-carboxylate

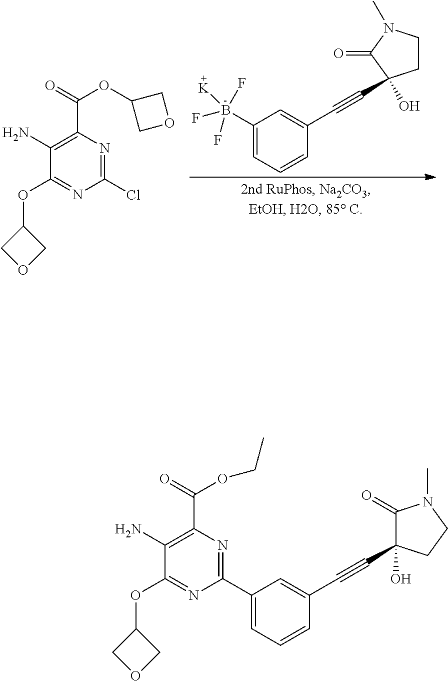

Similar to as described in General Procedure U, ethyl 5-amino-2-chloro-6-(oxetan-3-yloxy)pyrimidine-4-carboxylate was reacted with potassium (R)-trifluoro(3-((3-hydroxy-1-methyl-2-oxopyrrolidin-3-yl)ethynyl)phenyl)borate to give the title compound (200 mg, 48%) as a light yellow solid. LC-MS (ES, m/z): 453 [M+H]⁺.

### Step 4: Synthesis of 5-amino-2-(3-[2-[(3R)-3-hydroxy-1-methyl-2-oxopyrrolidin-3-yl]ethynyl]phenyl)-6-(oxetan-3-yloxy)pyrimidine-4-carboxamide

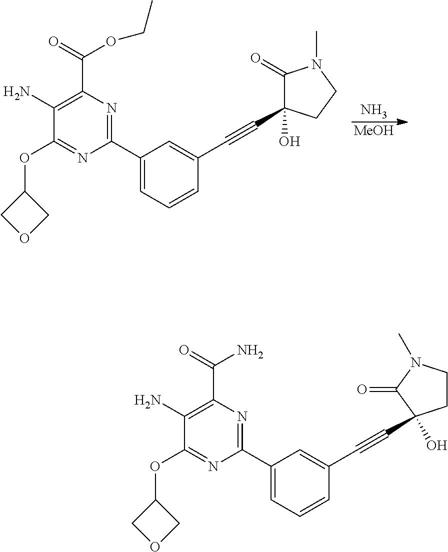

Similar to as described in General Procedure S, ethyl 5-amino-2-(3-[2-[(3R)-3-hydroxy-1-methyl-2-oxopyrrolidin-3-yl]ethynyl]phenyl)-6-(oxetan-3-yloxy)pyrimidine-4-carboxylate was reacted with ammonia to give the title compound (45.8 mg, 17%) as a white solid. LC-MS (ES, m/z): 424 [M+H]⁺. ¹H NMR (400 MHz, CD₃OD) δ 8.37 (s, 1H), 8.29 (d, J=7.6 Hz, 1H), 7.48-7.41 (m, 2H), 5.92-5.86 (m, 1H), 5.15 (t, J=7.0 Hz, 2H), 4.89-4.86 (m, 2H), 3.53-3.47 (m, 2H), 2.96 (s, 3H), 2.65-2.59 (m, 1H), 2.38-2.31 (m, 1H).

### Example A5

### Synthesis of 5-amino-2-(3-[2-[(3R)-3-hydroxy-1-methyl-2-oxopyrrolidin-3-yl]ethynyl]phenyl)-6-(oxan-4-yloxy)pyrimidine-4-carboxamide

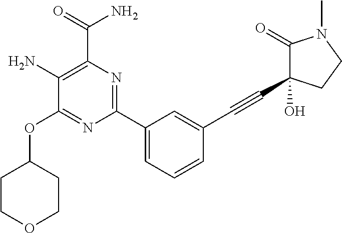 [SMILES: CN1CC[C@@](O)(C#Cc2cccc(-c3nc(OC4CCOCC4)c(N)c(C(N)=O)n3)c2)C1=O]

### Step 1: Synthesis of ethyl 5-amino-2-chloro-6-(tetrahydro-2H-pyran-4-yloxy)pyrimidine-4-carboxylate

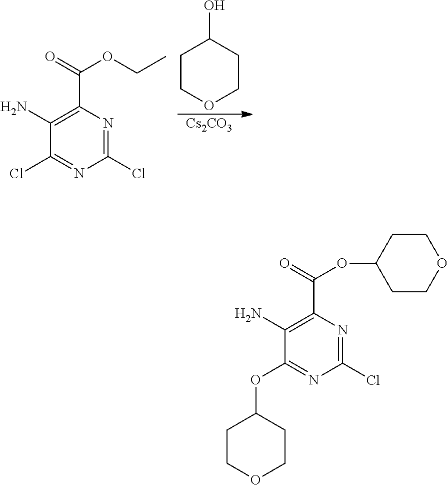

Similar to as described in General Procedure A, ethyl 5-amino-2,6-dichloropyrimidine-4-carboxylate was reacted with oxan-4-ol to give the title compound (600 mg, 99%) as a white solid. LC-MS (ES, m/z): 358 [M+H]⁺.

### Step 2: Synthesis of ethyl 5-amino-2-(3-[2-[(3R)-3-hydroxy-1-methyl-2-oxopyrrolidin-3-yl]ethynyl]phenyl)-6-(oxan-4yloxy)-pyrimidine-4-carboxylate

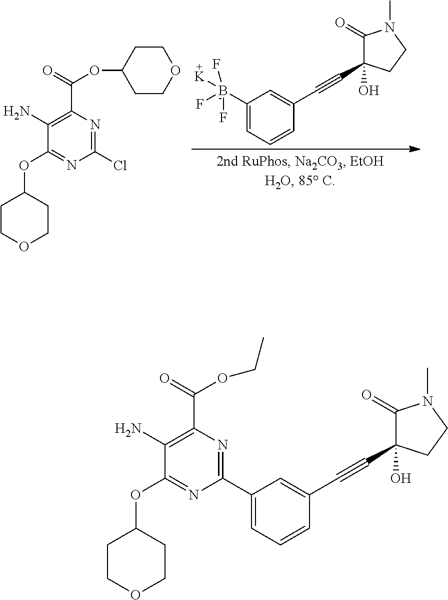

Similar to as described in General Procedure U, oxan-4-yl 5-amino-2-chloro-6-(oxan-4-yloxy)pyrimidine-4-carboxylate was reacted with potassium (R)-trifluoro(3-((3-hydroxy-1-methyl-2-oxopyrrolidin-3-yl)ethynyl)phenyl)borate to give the title compound (366 mg, 85%) as a light yellow solid. LC-MS (ES, m/z): 481 [M+H]⁺.

### Step 3: Synthesis of 5-amino-2-(3-[2-[(3R)-3-hydroxy-1-methyl-2-oxopyrrolidin-3-yl]ethynyl]phenyl)-6-(oxan-4-yloxy)pyrimidine-4-carboxamide

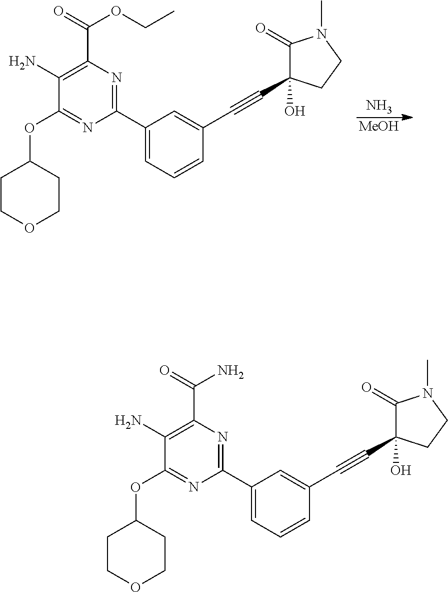

Similar to as described in General Procedure S, ethyl 5-amino-2-(3-[2-[(3R)-3-hydroxy-1-methyl-2-oxopyrrolidin-3-yl]ethynyl]phenyl)-6-(oxan-4-yloxy)pyrimidine-4-carboxylate was reacted with ammonia to give the title compound (25.9 mg, 8%) as a white solid. LC-MS (ES, m/z): 452 [M+H]⁺. ¹H NMR (400 MHz, CD₃OD) δ 8.37 (s, 1H), 8.35-8.32 (d, J=7.6 Hz, 1H), 7.49-7.41 (m, 2H), 5.60-5.58 (m, 1H), 4.08-4.03 (m, 2H), 3.76-3.70 (m, 2H), 3.52-3.49 (m, 2H), 2.96 (s, 3H), 2.62-2.60 (m, 1H), 2.36-2.32 (m, 1H), 2.24-2.20 (m, 2H), 1.96-1.92 (m, 2H).

### Example B5

### Synthesis of (R)-5-amino-2-(3-((3-hydroxy-1-methyl-2-oxopyrrolidin-3-yl)ethynyl)phenyl)-6-(2-m ethoxyethoxy)pyrimidine-4-carboxamide

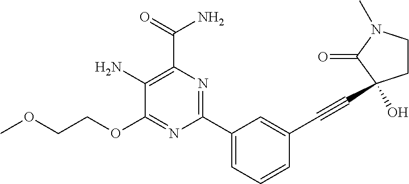 [SMILES: COCCOc1nc(-c2cccc(C#C[C@]3(O)CCN(C)C3=O)c2)nc(C(N)=O)c1N]

### Step 1: Synthesis of 5-amino-2-chloro-6-(2-methoxyethoxy)pyrimidine-4-carboxylate

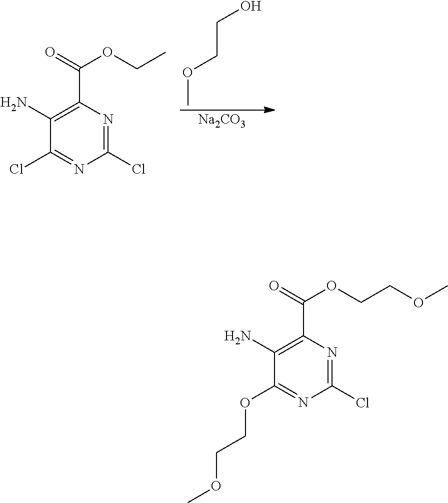

Similar to as described in General Procedure A, ethyl 5-amino-2,6-dichloropyrimidine-4-carboxylate was reacted with 2-methoxyethan-1-ol to give the title compound (216 mg, 65%) as a light yellow solid. LC-MS (ES, m/z): 306 [M+H]⁺.

### Step 2: Synthesis of 5-amino-2-(3-[2-[(3R)-3-hydroxy-1-methyl-2-oxopyrrolidin-3-yl]ethynyl]phenyl)-6-(2-methoxyethoxy)pyrimidine-4-carboxylate

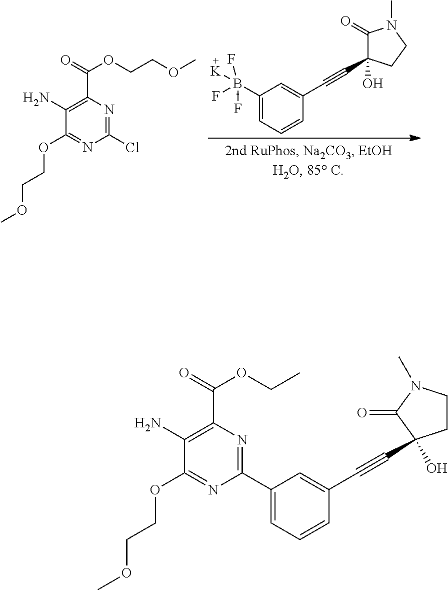

Similar to as described in General Procedure U, 2-methoxyethyl 5-amino-2-chloro-6-(2-methoxyethoxy)pyrimidine-4-carboxylate was reacted with potassium (R)-trifluoro(3-((3-hydroxy-1-methyl-2-oxopyrrolidin-3-yl)ethynyl)phenyl)borate to give the title compound (287 mg, 48%) as a light yellow solid. LC-MS (ES, m/z): 455 [M+H]⁺.

### Step 3: Synthesis of 5-amino-2-(3-[2-[(3R)-3-hydroxy-1-methyl-2-oxopyrrolidin-3-yl]ethynyl]phenyl)-6-(2-methoxyethoxy)pyrimidine-4-carboxamide

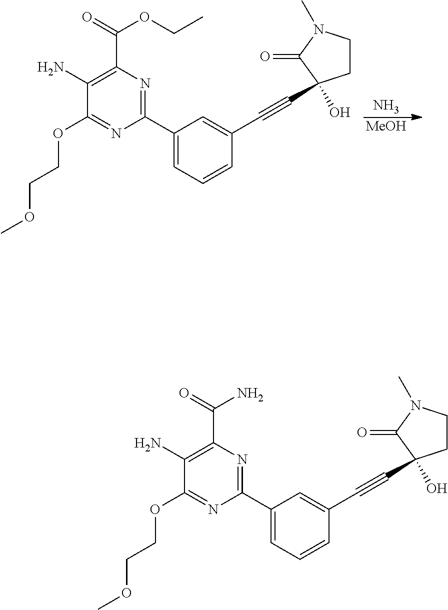

Similar to as described in General Procedure S, ethyl 5-amino-2-(3-[2-[(3R)-3-hydroxy-1-methyl-2-oxopyrrolidin-3-yl]ethynyl]phenyl)-6-(2-methoxyethoxy)pyrimidine-4-carboxylate was reacted with ammonia to give the title compound (13.5 mg, 5%) as a white solid. LC-MS (ES, m/z): 426 [M+H]⁺. ¹H NMR (300 MHz, CD₃OD) δ 8.41 (s, 1H), 8.34 (d, J=7.5 Hz, 1H), 7.46-7.37 (m, 2H), 4.73-4.70 (t, J=4.5 Hz, 2H), 3.87 (t, J=4.5 Hz, 2H), 3.50-3.43 (m, 5H), 2.92 (s, 1H), 2.63-2.55 (m, 1H), 2.35-2.26 (m, 1H).

### Example C5

### Synthesis of (R)-2-(2-fluoro-5-((3-hydroxy-1-methyl-2-oxopyrrolidin-3-yl)ethynyl)phenyl)-5-(2-methoxyethylamino)pyrimidine-4-carboxamide

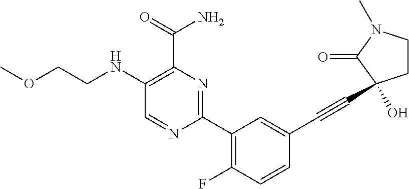 [SMILES: COCCNc1cnc(-c2cc(C#C[C@]3(O)CCN(C)C3=O)ccc2F)nc1C(N)=O]

### Step 1: Synthesis of ethyl 2-(5-bromo-2-fluorophenyl)-5-fluoropyrimidine-4-carboxylate

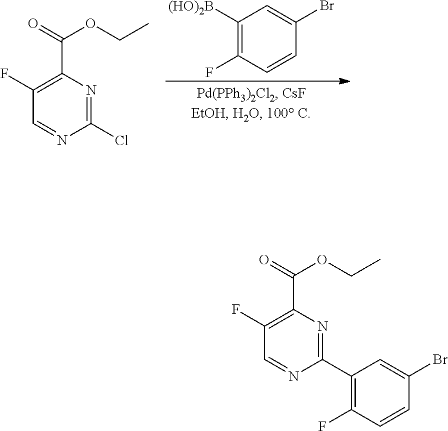 [SMILES: CCOC(=O)c1nc(Cl)ncc1F ; OB(O)c1cc(Br)ccc1F ; CCOC(=O)c1nc(-c2cc(Br)ccc2F)ncc1F]

Similar to as described in General Procedure X, ethyl 2-chloro-5-fluoropyrimidine-4-carboxylate was reacted with 5-bromo-2-fluorophenylboronic acid to give the title compound (310 mg, 37%) as colorless oil. LC-MS (ES, m/z): 343, 345 [M+H]⁺.

### Step 2: Synthesis of ethyl 2-(5-bromo-2-fluorophenyl)-5-[(2-methoxyethyl)amino]pyrimidine-4-carboxylate

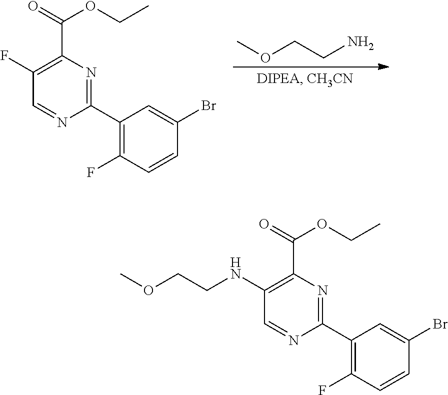

Similar to as described in General Procedure A, ethyl 2-(5-bromo-2-fluorophenyl)-5-fluoropyrimidine-4-carboxylate was reacted with 2-methoxyethan-1-amine to give the title compound (400 mg, 78%) as a yellow solid. LC-MS (ES, m/z): 398, 400 [M+H]⁺.

### Step 3: Synthesis of ethyl 2-(2-fluoro-5-[2-[(3R)-3-hydroxy-1-methyl-2-oxopyrrolidin-3-yl]ethynyl]phenyl)-5-[(2-methoxyethyl)amino]pyrimidine-4-carboxylate

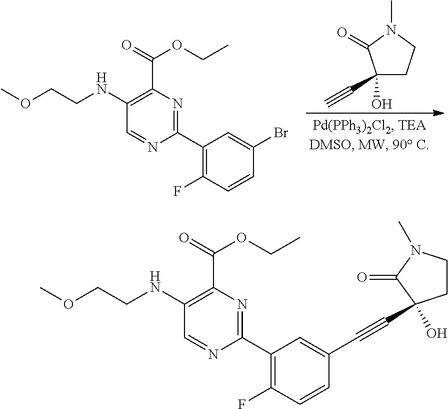

Similar to as described in General Procedure G, 2-(5-bromo-2-fluorophenyl)-5-[(2-methoxyethyl)amino]pyrimidine-4-carboxylate was reacted with (R)-3-ethynyl-3-hydroxy-1-methylpyrrolidin-2-one to give the title compound (355 mg, 77%) as a yellow solid. LC-MS (ES, m/z): 457 [M+H]⁺.

### Step 4: Synthesis of 2-(2-fluoro-5-[2-[(3R)-3-hydroxy-1-methyl-2-oxopyrrolidin-3-yl]ethynyl]phenyl)-5-[(2-methoxyethyl)amino]pyrimidine-4-carboxamide

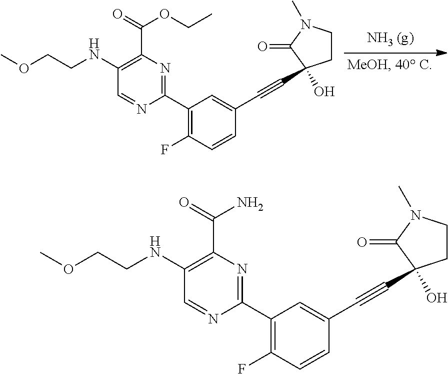 [SMILES: CCOC(=O)c1nc(-c2cc(C#C[C@]3(O)CCN(C)C3=O)ccc2F)ncc1NCCOC ; COCCNc1cnc(-c2cc(C#C[C@]3(O)CCN(C)C3=O)ccc2F)nc1C(N)=O]

Similar to as described in General Procedure S, ethyl 2-(2-fluoro-5-[2-[(3R)-3-hydroxy-1-methyl-2-oxopyrrolidin-3-yl]ethynyl]phenyl)-5-[(2-methoxyethyl)amino]pyrimidine-4-carboxylate was reacted with ammonia to give the title compound (49.3 mg, 15%) as a yellow solid. LC-MS (ES, m/z): 428 [M+H]⁺. ¹H NMR (300 MHz, CD₃OD) δ 8.63 (s, 1H), 8.16 (dd, J=7.5, 2.1 Hz, 1H), 7.54-7.49 (m, 1H), 7.21 (dd, J=10.8, 5.4 Hz, 1H), 3.68 (t, J=2.1 Hz, 2H), 3.61-3.46 (m, 4H), 3.42 (s, 3H), 2.93 (s, 3H), 2.63-2.45 (m, 1H), 2.36-2.29 (m, 1H).

### Example D5

### Synthesis of 5-amino-2-(2-fluoro-5-[2-[(3R)-3-hydroxy-1-methyl-2-oxopyrrolidin-3-yl]ethynyl]phenyl)pyrimidine-4-carboxamide

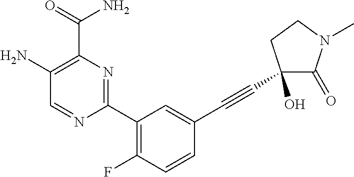 [SMILES: CN1CC[C@@](O)(C#Cc2ccc(F)c(-c3ncc(N)c(C(N)=O)n3)c2)C1=O]

### Step 1: Synthesis of 2-chloro-4-(1-ethoxyethenyl)-5-fluoropyrimidine

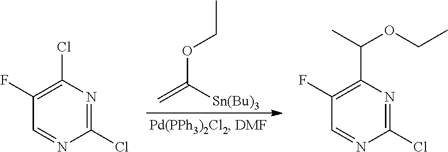

Similar to as described in General Procedure Q, 2,4-dichloro-5-fluoropyrimidine was reacted with tributyl(1-ethoxyethenyl)stannane to give the title compound (600 mg, 49%) as an yellow oil. LC-MS (ES, m/z): 203 [M+H]⁺.

### Step 2: Synthesis of ethyl 2-chloro-5-fluoropyrimidine-4-carboxylate

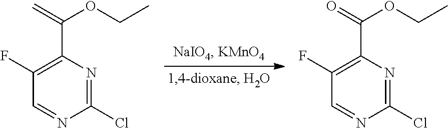

Similar to as described in General Procedure R, 2-chloro-4-(1-ethoxyethenyl)-5-fluoropyrimidine was reacted with potassium permanganate to give the title compound (400 mg, 66%) as a light yellow oil. LC-MS (ES, m/z): 205 [M+H]⁺.

### Step 3: Synthesis of ethyl 2-(5-bromo-2-fluorophenyl)-5-fluoropyrimidine-4-carboxylate

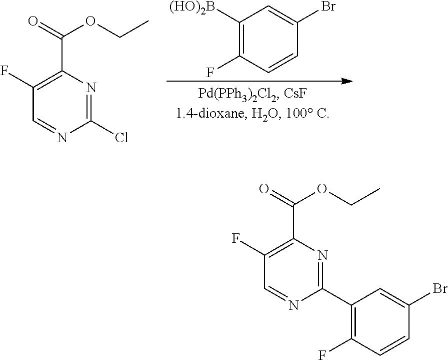 [SMILES: CCOC(=O)c1nc(Cl)ncc1F ; OB(O)c1cc(Br)ccc1F ; CCOC(=O)c1nc(-c2cc(Br)ccc2F)ncc1F]

Similar to as described in General Procedure X, ethyl 2-chloro-5-fluoropyrimidine-4-carboxylate was reacted with 5-bromo-2-fluorophenylboronic acid to give the title compound (150 mg, 22%) as a colorless oil. LC-MS (ES, m/z): 343, 345 [M+H]⁺.

### Step 4: Synthesis of 5-amino-2-(5-bromo-2-fluorophenyl)pyrimidine-4-carboxamide

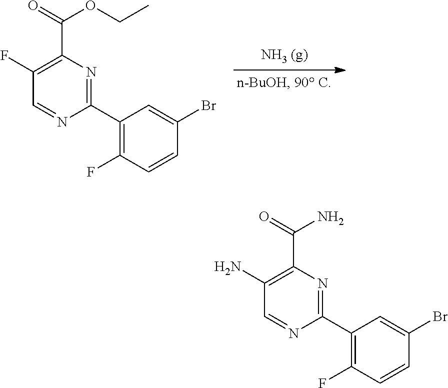 [SMILES: CCOC(=O)c1nc(-c2cc(Br)ccc2F)ncc1F ; NC(=O)c1nc(-c2cc(Br)ccc2F)ncc1N]

Similar to as described in General Procedure S, ethyl 2-(5-bromo-2-fluorophenyl)-5-fluoropyrimidine-4-carboxylate (150 mg, 0.44 mmol, 1.00 equiv) was reacted with ammonia to give the title compound (70 mg, 51%) as a light yellow solid. LC-MS (ES, m/z): 311, 313 [M+H]⁺.

### Step 5: Synthesis of 5-amino-2-(2-fluoro-5-[2-[(3R)-3-hydroxy-1-methyl-2-oxopyrrolidin-3-yl]ethynyl]phenyl)pyrimidine-4-carboxamide

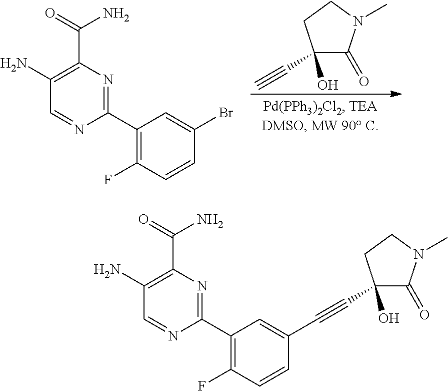

Similar to as described in General Procedure G, 5-amino-2-(5-bromo-2-fluorophenyl)pyrimidine-4-carboxamide was reacted with (R)-3-ethynyl-3-hydroxy-1-methylpyrrolidin-2-one to give the title compound (31.3 mg, 38%) as a white solid. LC-MS (ES, m/z): 370 [M+H]⁺. ¹H NMR (400 MHz, CD₃OD) δ 8.53 (s, 1H), 8.18 (d, J=8 Hz, 1H), 7.53-7.49 (m, 1H), 7.20-4.25 (m, 1H), 3.47-3.49 (m, 2H), 2.95 (s, 3H), 2.63-2.58 (m, 1H), 2.36-2.29 (m, 1H).

### Example E5

### Synthesis of 6-(3-[2-[(3R)-3-hydroxy-1-methyl-2-oxopyrrolidin-3-yl]ethynyl]phenyl)-4-(5-methyl-1H-pyrazol-1-yl)pyridine-2-carboxamide

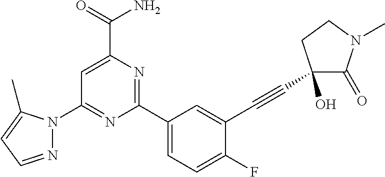 [SMILES: Cc1ccnn1-c1cc(C(N)=O)nc(-c2ccc(F)c(C#C[C@]3(O)CCN(C)C3=O)c2)n1]

### Step 1: Synthesis of methyl 2-chloro-6-hydrazinylpyrimidine-4-carboxylate

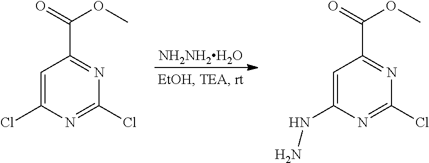

Similar to as described in General Procedure A, methyl 2,6-dichloropyrimidine-4-carboxylate was reacted with hydrazine hydrate to give the title compound (1.5 g, 77%) as a yellow solid. LC-MS (ES, m/z): 203 [M+H]⁺.

### Step 2: Synthesis of 2-chloro-6-(5-methyl-1H-pyrazol-1-yl)pyrimidine-4-carboxylate

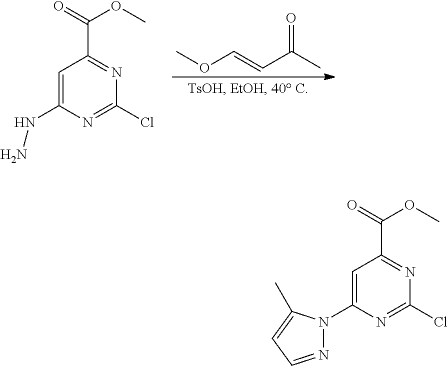

A suspension of methyl 2-chloro-6-hydrazinylpyrimidine-4-carboxylate (1.5 g, 7.40 mmol, 1.00 equiv), (3E)-4-methoxybut-3-en-2-one (739 mg, 7.38 mmol, 1.00 equiv), p-toluenesulfonic acid (127 mg, 0.74 mmol, 0.10 equiv) in ethanol (30 mL) was stirred for 18 h at 40° C. After completion the reaction was concentrated under vacuum and the residue was purified by a silica gel column eluting with ethyl acetate/petroleum ether (1:20) to give the title compound (810 mg, 43%) as a white solid. LC-MS (ES, m/z): 253 [M+H]⁺.

### Step 3: Synthesis of methyl 2-(3-bromo-4-fluorophenyl)-6-(5-methyl-1H-pyrazol-1-yl)pyrimidine-4-carboxylate

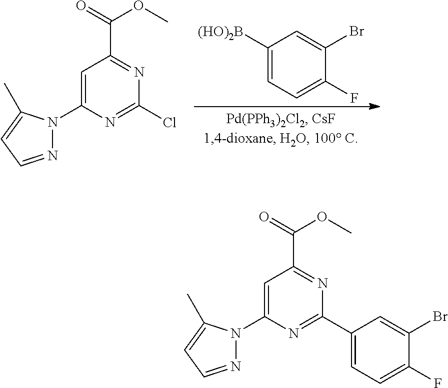 [SMILES: COC(=O)c1cc(-n2nccc2C)nc(Cl)n1 ; OB(O)c1ccc(F)c(Br)c1 ; COC(=O)c1cc(-n2nccc2C)nc(-c2ccc(F)c(Br)c2)n1]

Similar to as described in General Procedure X, methyl 2-chloro-6-(5-methyl-1H-pyrazol-1-yl)pyrimidine-4-carboxylate was reacted with 3-bromo-4-fluorophenylboronic acid to give the title compound (200 mg, 26%) as a white solid. LC-MS (ES, m/z): 391, 393 [M+H]⁺.

### Step 4: Synthesis of methyl 2-(4-fluoro-3-[2-[(3R)-3-hydroxy-1-methyl-2-oxopyrrolidin-3-yl]ethynyl]phenyl)-6-(5-methyl-1H-pyrazol-1-yl)pyrimidine-4-carboxylate

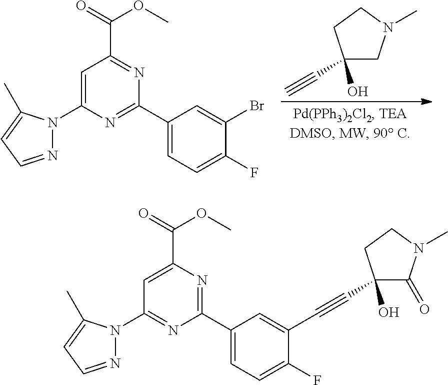

Similar to as described in General Procedure G, methyl 2-(3-bromo-4-fluorophenyl)-6-(5-methyl-1H-pyrazol-1-yl)pyrimidine-4-carboxylate was reacted with (R)-3-ethynyl-3-hydroxy-1-methylpyrrolidin-2-one to give the title compound (150 mg, 65%) as a yellow solid. LC-MS (ES, m/z): 450 [M+H]⁺.

### Step 5: Synthesis of 6-(3-[2-[(3R)-3-hydroxy-1-methyl-2-oxopyrrolidin-3-yl]ethynyl]phenyl)-4-(5-methyl-1H-pyrazol-1-yl)pyridine-2-carboxamide

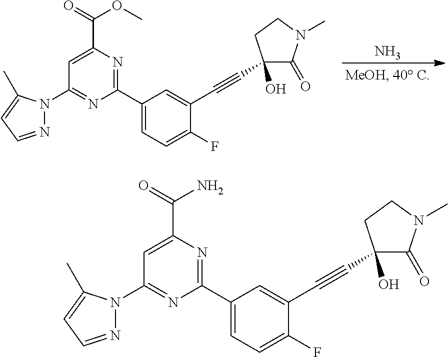

Similar to as described in General Procedure S, methyl 6-(3-[2-[(3R)-3-hydroxy-1-methyl-2-oxopyrrolidin-3-yl]ethynyl]phenyl)-4-(5-methyl-1H-pyrazol-1-yl)pyridine-2-carboxylate was reacted with ammonia to give the title compound (39.9 mg, 28%) as a light yellow solid. LC-MS (ES, m/z): 435 [M+H]⁺. ¹H NMR (400 MHz, CD₃OD) δ 8.87-8.58 (m, 2H), 8.50-8.49 (m, 1H), 7.74 (s, 1H), 7.38-7.35 (m, 1H), 6.40 (s, 1H), 3.54-3.51 (m, 2H), 2.97 (s, 3H), 2.93 (s, 3H), 2.65-2.53 (m, 1H), 2.41-2.36 (m, 1H).

### Example F5

### Synthesis of 5-amino-8-(3-[2-[(3R)-3-hydroxy-1-methyl-2-oxopyrrolidin-3-yl]ethynyl]phenyl)imidazo[1,2-a]pyrazine-6-carboxamide

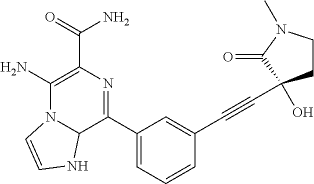 [SMILES: CN1CC[C@@](O)(C#Cc2cccc(C3=NC(C(N)=O)=C(N)N4C=CNC34)c2)C1=O]

### Step 1: Synthesis of methyl 5-amino-8-chloroimidazo[1,2-a]pyrazine-6-carboxylate

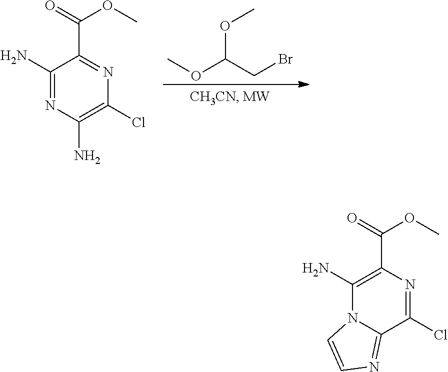

A solution of methyl 3,5-diamino-6-chloropyrazine-2-carboxylate (1.2 g, 5.92 mmol, 1.00 equiv) and 2-bromo-1,1-dimethoxyethane (2 g, 11.83 mmol, 2.00 equiv) in acetonitrile (20 mL) was irradiated with microwave for 40 min at 120° C. After completion the resulting solution was concentrated under vacuum and the residue was purified by a silica gel column eluting with dichloromethane/methanol (10:1) to give the title compound (300 mg, 22%) as a yellow solid. LC-MS (ES, m/z): 227 [M+H]⁺.

### Step 2: Synthesis of 5-amino-8-(3-[2-[(3R)-3-hydroxy-1-methyl-2-oxopyrrolidin-3-yl]ethynyl]phenyl)imidazo[1,2-a]pyrazine-6-carboxamide

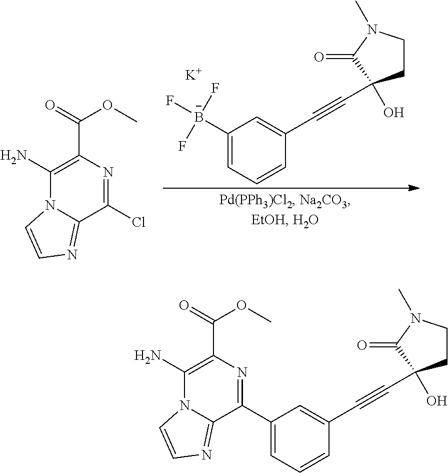 [SMILES: COC(=O)c1nc(Cl)c2nccn2c1N ; CN1CC[C@@](O)(C#Cc2cccc([B-](F)(F)F)c2)C1=O ; [K+] ; COC(=O)c1nc(-c2cccc(C#C[C@]3(O)CCN(C)C3=O)c2)c2nccn2c1N]

Similar to as described in General Procedure U, 5-amino-8-chloroimidazo[1,2-a]pyrazine-6-carboxamide was reacted with potassium (R)-trifluoro(3-((3-hydroxy-1-methyl-2-oxopyrrolidin-3-yl)ethynyl)phenyl)borate to give the title compound (80 mg, 29%) as a yellow solid. LC-MS (ES, m/z): 406 [M+H]⁺.

### Step 3: Synthesis of 5-amino-8-(3-[2-[(3R)-3-hydroxy-1-methyl-2-oxopyrrolidin-3-yl]ethynyl]phenyl)imidazo[1,2-a]pyrazine-6-carboxamide

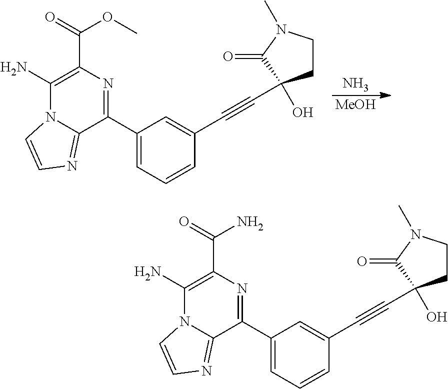

Similar to as described in General Procedure S, methyl 5-amino-8-(3-[2-[(3R)-3-hydroxy-1-methyl-2-oxopyrrolidin-3-yl]ethynyl]phenyl)imidazo[1,2-a]pyrazine-6-carboxylate was reacted with ammonia to give the title compound (8 mg, 17%) as an off-white solid. LC-MS (ES, m/z): 391 [M+H]⁺. ¹H NMR (300 MHz, CD₃OD) δ 8.65 (s, 1H), 8.56-8.52 (m, 1H), 8.17 (d, J=11.1 Hz, 1H), 7.82 (d, J=1.5 Hz, 1H), 7.51-7.49 (m, 2H), 3.54-3.48 (m, 2H), 2.94 (s, 3H), 2.65-2.59 (m, 1H), 2.37-2.27 (m, 1H).

### Example G5

### Synthesis of 8-(3-[2-[(3R)-3-hydroxy-1-methyl-2-oxopyrrolidin-3-yl]ethynyl]phenyl)-3-(trifluoromethyl)imidazo[1,2-a]pyrazine-6-carboxamide

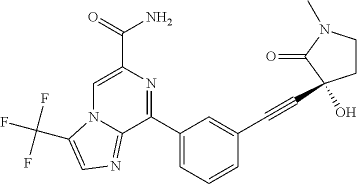 [SMILES: CN1CC[C@@](O)(C#Cc2cccc(-c3nc(C(N)=O)cn4c(C(F)(F)F)cnc34)c2)C1=O]

### Step 1: Synthesis of methyl 5-amino-6-bromopyrazine-2-carboxylate

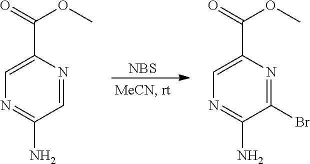

A suspension of methyl 5-aminopyrazine-2-carboxylate (2.0 g, 13.06 mmol, 1.00 equiv) and N-bromosuccinimide (2.79 g, 15.68 mmol, 1.20 equiv) in acetonitrile (30 mL) was stirred for 5 h at room temperature. After completion the resulting solution was concentrated under vacuum and the residue was purified by a silica gel column chromatography eluting with ethyl acetate/petroleum ether (1:1) to give the title compound (1.42 g, 47%) as a light yellow solid. LC-MS (ES, m/z): 232, 234 [M+H]⁺.

### Step 2: Synthesis of methyl 8-bromoimidazo[1,2-a]pyrazine-6-carboxylate

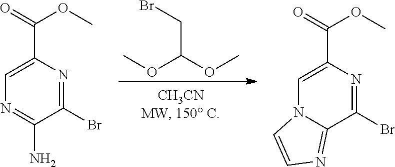

A solution of methyl 5-amino-6-bromopyrazine-2-carboxylate (600 mg, 2.59 mmol, 1.00 equiv) and 2-bromo-1,1-dimethoxyethane (1.73 g, 10.24 mmol, 4.00 equiv) in acetonitrile (10 mL) was irradiated with microwave for 1 h at 150° C. After completion the resulting solution was concentrated under vacuum and the residue was purified by a silica gel column chromatography eluting with ethyl acetate/petroleum ether (1:5) to give the title compound (411 mg, 62%) as a light brown solid. LC-MS (ES, m/z): 256, 258 [M+H]⁺.

### Step 3: Synthesis of

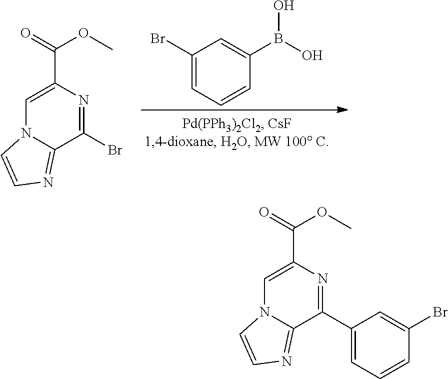 [SMILES: COC(=O)c1cn2ccnc2c(Br)n1 ; OB(O)c1cccc(Br)c1 ; COC(=O)c1cn2ccnc2c(-c2cccc(Br)c2)n1]

Similar to as described in General Procedure X, methyl 8-bromoimidazo[1,2-a]pyrazine-6-carboxylate was reacted with 3-bromophenylboronic acid to give the title compound (250 mg, 47%) as a light yellow solid. LC-MS (ES, m/z): 322, 324 [M+H]⁺.

### Step 4: Synthesis of methyl 8-(3-bromophenyl)-3-iodoimidazo[1,2-a]pyrazine-6-carboxylate

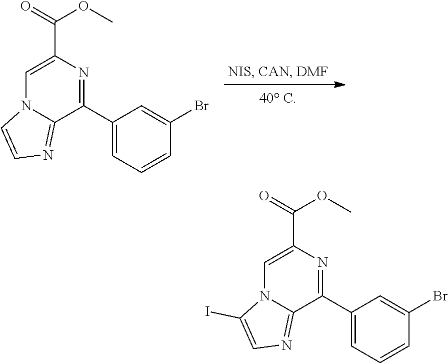 [SMILES: COC(=O)c1cn2ccnc2c(-c2cccc(Br)c2)n1 ; COC(=O)c1cn2c(I)cnc2c(-c2cccc(Br)c2)n1]

A solution of methyl 8-(3-bromophenyl)imidazo[1,2-a]pyrazine-6-carboxylate (250 mg, 0.75 mmol, 1.00 equiv), N-iodosuccinimide (338 mg, 1.50 mmol, 2.00 equiv), and ceric ammonium nitrate (82.5 mg, 0.15 mmol, 0.20 equiv) in DMF (6 mL) was reacted at 40° C. for 12 h. After completion the reaction was quenched by water and extracted with ethyl acetate. The organic layers were dried over anhydrous sodium sulfate and concentrated under vacuum. The residue was purified by silica gel column chromatography eluting with ethyl acetate/petroleum ether (1:5) to give the title compound (292 mg, 85%) as a light yellow solid. LC-MS (ES, m/z): 458, 460 [M+H]⁺.

### Step 5: Synthesis of methyl 8-(3-bromophenyl)-3-(trifluoromethyl)imidazo[1,2-a]pyrazine-6-carboxylate

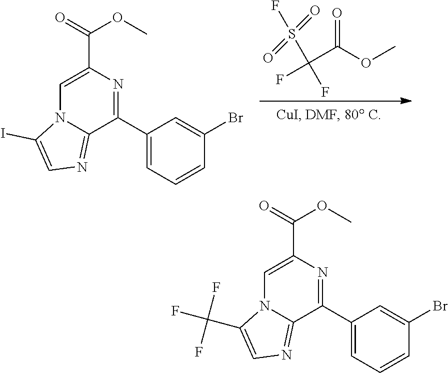 [SMILES: COC(=O)c1cn2c(I)cnc2c(-c2cccc(Br)c2)n1 ; COC(=O)C(F)(F)S(=O)(=O)F ; COC(=O)c1cn2c(C(F)(F)F)cnc2c(-c2cccc(Br)c2)n1]

A mixture of methyl 8-(3-bromophenyl)imidazo[1,2-a]pyrazine-6-carboxylate (250 mg, 0.75 mmol, 1.00 equiv), copper(I) iodide (518 mg, 2.72 mmol, 3.60 equiv), and methyl 2,2-difluoro-2-(fluorosulfonyl)acetate (524 mg, 2.73 mmol, 3.60 equiv) in DMF (5 mL) was stirred for 8 h at 80° C. After completion the reaction was quenched with water and extracted with ethyl acetate. The organic layers were combined, dried over anhydrous sodium sulfate, and concentrated under vacuum. The residue was purified by a silica gel column eluting with ethyl acetate/petroleum ether (1:10) to give the title compound (201 mg, 67%) as a light yellow solid. LC-MS (ES, m/z): 400, 402 [M+H]⁺.

### Step 6: Synthesis of 8-(3-bromophenyl)-3-(trifluoromethyl)imidazo[1,2-a]pyrazine-6-carboxamide

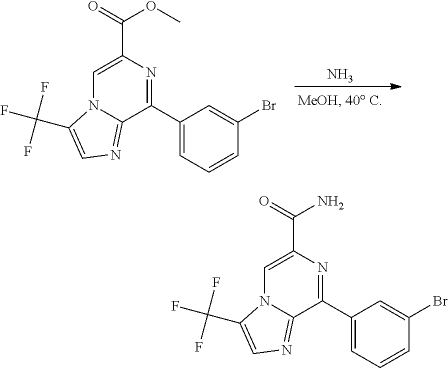

Similar to as described in General Procedure S, methyl 8-(3-bromophenyl)-3-(trifluoromethyl)imidazo[1,2-a]pyrazine-6-carboxylate was reacted with ammonia to give the title compound (152 mg, crude) as a light brown solid. LC-MS (ES, m/z): 385, 387 [M+H]⁺.

### Step 7: Synthesis of 8-(3-[2-[(3R)-3-hydroxy-1-methyl-2-oxopyrrolidin-3-yl]ethynyl]phenyl)-3-(trifluoromethyl)imidazo[1,2-a]pyrazine-6-carboxamide

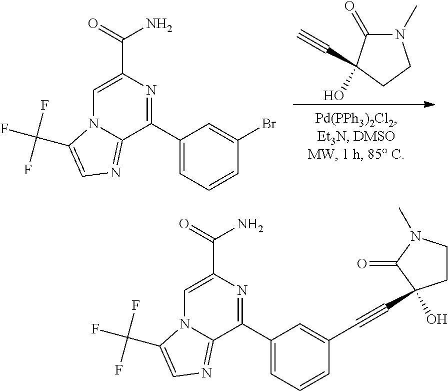

Similar to as described in General Procedure G, 8-(3-bromophenyl)-3-(trifluoromethyl)imidazo[1,2-a]pyrazine-6-carboxamide was reacted with (R)-3-ethynyl-3-hydroxy-1-methylpyrrolidin-2-one to give the title compound (54.8 mg, 26%) as a white solid. LC-MS (ES, m/z): 444 [M+H]⁺. ¹H NMR (400 MHz, CD₃OD) δ 9.03 (s, 1H), 8.99 (s, 1H), 8.87 (d, J=7.6 Hz, 1H), 8.39 (s, 1H), 7.69 (d, J=7.6 Hz, 1H), 7.60 (t, J=7.6 Hz, 1H), 3.56-3.47 (m, 2H), 2.96 (s, 3H), 2.67-2.61 (m, 1H), 2.39-2.32 (m, 1H).

### Example H5

### Synthesis of 1-ethyl-4-(3-[2-[(3R)-3-hydroxy-1-methyl-2-oxopyrrolidin-3-yl]ethynyl]phenyl)-1H-pyrazolo[4,3-c]pyridine-6-carboxamide

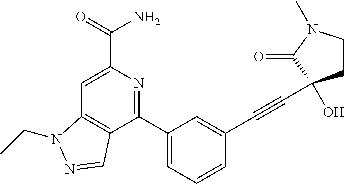 [SMILES: CCn1ncc2c(-c3cccc(C#C[C@]4(O)CCN(C)C4=O)c3)nc(C(N)=O)cc21]

### Step 1: Synthesis of 4,6-dichloro-1-ethyl-1H-pyrazolo[4,3-c]pyridine

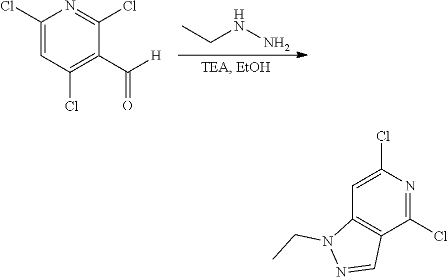

Ethylhydrazine (2.16 g, 14.39 mmol, 1.00 equiv) was added to a solution of 2,4,6-trichloropyridine-3-carbaldehyde (3 g, 14.26 mmol, 1.00 equiv) and triethylamine (4.3 g, 42.49 mmol, 3.00 equiv) in ethanol (100 mL) at −78° C. under nitrogen. The resulting solution was stirred for 3 hours at 0° C. After completion the reaction was concentrated under vacuum and the residue was purified by a silica gel column eluting with ethyl acetate/petroleum ether (1:20) to give the title compound (800 mg, 26%) as a white solid. LC-MS (ES, m/z): 216 [M+H]⁺.

### Step 2: Synthesis of (3R)-3-[2-(3-[6-chloro-1-ethyl-1H-pyrazolo[4,3-c]pyridin-4-yl]phenyl)ethynyl]-3-hydroxy-1-methyl pyrrolidin-2-one

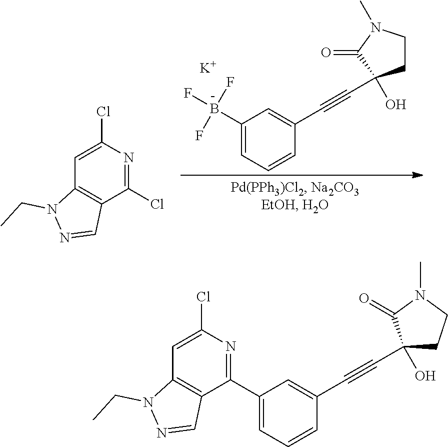

Similar to as described in General Procedure U, 4,6-dichloro-1-ethyl-1H-pyrazolo[4,3-c]pyridine was reacted with potassium (R)-trifluoro(3-((3-hydroxy-1-methyl-2-oxopyrrolidin-3-yl)ethynyl)phenyl)borate to give the title compound (340 mg, 25%) as a yellow solid. LC-MS (ES, m/z): 395 [M+H]⁺.

### Step 3: Synthesis of methyl 1-ethyl-4-(3-[2-[(3R)-3-hydroxy-1-methyl-2-oxopyrrolidin-3-yl]ethynyl]phenyl)-1H-pyrazolo[4,3-c]pyridine-6-carboxylate

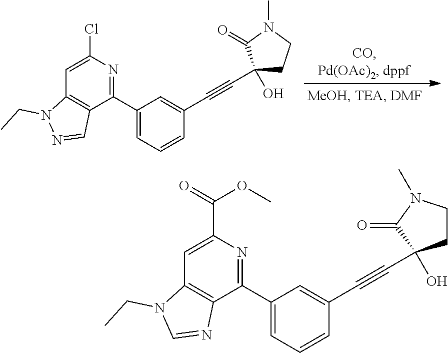

Similar to as described in General Procedure O, (3R)-3-[2-(3-[6-chloro-1-ethyl-1H-pyrazolo[4,3-c]pyridin-4-yl]phenyl)ethynyl]-3-hydroxy-1-methylpyrrolidin-2-one was reacted with carbon monoxide to give the title compound (120 mg, 45%) as a yellow solid. LC-MS (ES, m/z): 419 [M+H]⁺.

### Step 4: Synthesis of 1-ethyl-4-(3-[2-[(3R)-3-hydroxy-1-methyl-2-oxopyrrolidin-3-yl]ethynyl]phenyl)-1H-pyrazolo[4,3-c]pyridine-6-carboxamide

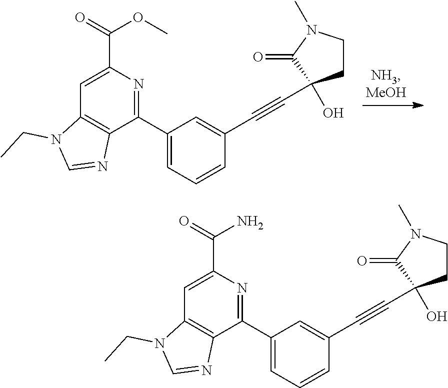

Similar to as described in General Procedure S, methyl 1-ethyl-4-(3-[2-[(3R)-3-hydroxy-1-methyl-2-oxopyrrolidin-3-yl]ethynyl]phenyl)-1H-pyrazolo[4,3-c]pyridine-6-carboxylate was reacted with ammonia to give the title compound (21.5 mg, 19%) as a white solid. LC-MS (ES, m/z): 404 [M+H]⁺. ¹H NMR (400 MHz, DMSO-d₆) δ 8.59 (s, 1H), 8.37 (s, 1H), 8.27-8.26 (m, 2H), 7.79 (s, 1H), 7.61-7.60 (m, 2H), 6.51 (s, 1H), 4.62-4.60 (m, 2H), 3.37-3.35 (m, 2H), 2.81 (s, 3H), 2.49-2.48 (m, 1H), 2.20-2.15 (m, 1H), 1.45 (t, J=7.2 Hz, 3H).

### Example I5

### Synthesis of 4-(3-[2-[(3R)-3-hydroxy-1-methyl-2-oxopyrrolidin-3-yl]ethynyl]phenyl)-1-(propan-2-yl)-1H-pyrazolo[4,3-c]pyridine-6-carboxamide

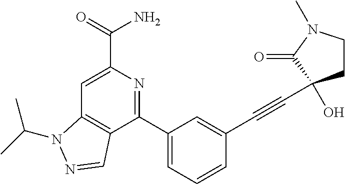 [SMILES: CC(C)n1ncc2c(-c3cccc(C#C[C@]4(O)CCN(C)C4=O)c3)nc(C(N)=O)cc21]

### Step 1: Synthesis of 2,4,6-trichloropyridine-3-carbaldehyde

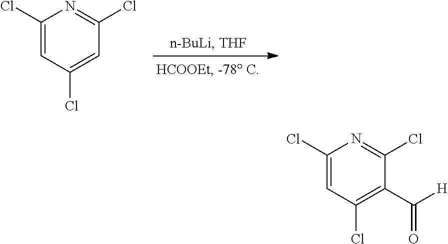 [SMILES: Clc1cc(Cl)nc(Cl)c1 ; O=Cc1c(Cl)cc(Cl)nc1Cl]

n-BuLi (2M, 15 mL, 1.00 equiv) was added to a solution of 2,4,6-trichloropyridine (6 g, 32.89 mmol, 1.00 equiv) in THF (100 mL) at −78° C. under nitrogen. After being stirred for 0.5 h at −78° C. ethyl formate (3.192 g, 43.09 mmol, 1.30 equiv) was added and the resulting solution was stirred for 3 h at −78° C. After completion the reaction was quenched with ammonium chloride solution, extracted with ethyl acetate, dried over anhydrous sodium sulfate, and concentrated under vacuum. The residue was purified by a silica gel column eluting with ethyl acetate/petroleum ether (1:20) to give the title compound (6 g, 87%) as a white solid.

### Step 2: Synthesis of 4,6-dichloro-1H-pyrazolo[4,3-c]pyridine

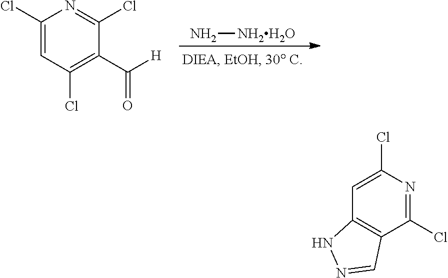

Under nitrogen hydrazine hydrate (3.8 g, 75.91 mmol, 4.00 equiv) was added to a solution of 2,4,6-trichloropyridine-3-carbaldehyde (4 g, 19.01 mmol, 1.00 equiv) and N-ethyl-N-isopropylpropan-2-amine (7.5 g, 58.03 mmol, 3.10 equiv) in ethanol (100 mL) at −20° C. The resulting solution was stirred for 16 h at −20° C. and 16 hours at 30° C. The resulting solution was concentrated under vacuum and the residue was purified by a silica gel column chromatography eluting with ethyl acetate/petroleum ether (1:2). This resulted in the title compound (1.6 g, 45%) as a white solid. LC-MS (ES, m/z): 188 [M+H]⁺.

### Step 3: Synthesis of 4,6-di chloro-1-(propan-2-yl)-1H-pyrazolo[4,3-c]pyridine

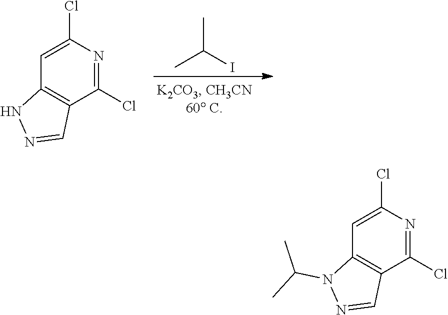

A solution of 4,6-dichloro-1H-pyrazolo[4,3-c]pyridine (510 mg, 2.71 mmol, 1.00 equiv), potassium carbonate (714 mg, 5.17 mmol, 1.90 equiv), and 2-iodopropane (867 mg, 5.10 mmol, 1.90 equiv) in acetonitrile (20 mL) was stirred for 16 h at 60° C. The resulting solution was concentrated under vacuum and the residue was purified by a silica gel column eluting with ethyl acetate/petroleum ether (1:9). This resulted in the title compound (250 mg, 40%) as a white solid. LC-MS (ES, m/z): 230 [M+H]⁺.

### Step 4: Synthesis of (3R)-3-(2-[3-[6-chloro-1-(propan-2-yl)-1H-pyrazolo[4,3-c]pyridin-4-yl]phenyl]ethynyl)-3-hydroxy-1-methylpyrrolidin-2-one

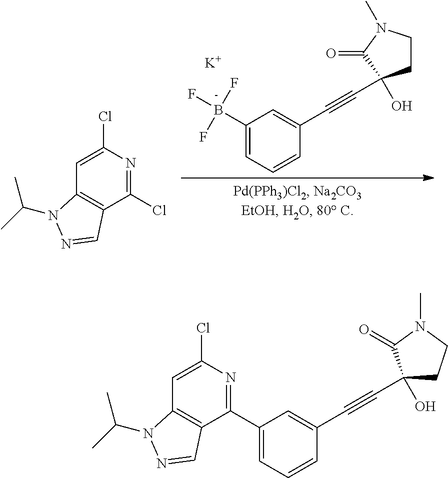

Similar to as described in General Procedure U, 4,6-dichloro-1-(propan-2-yl)-1H-pyrazolo[4,3-c]pyridine was reacted with potassium (R)-trifluoro(3-((3-hydroxy-1-methyl-2-oxopyrrolidin-3-yl)ethynyl)phenyl)borate to give the title compound (450 mg, 63%) as a brown solid. LC-MS (ES, m/z): 409 [M+H]⁺.

### Step 5: Synthesis of methyl 4-(3-[2-[(3R)-3-hydroxy-1-methyl-2-oxopyrrolidin-3-yl]ethynyl]phenyl)-1-(propan-2-yl)-1H-pyrazolo[4,3-c]pyridine-6-carboxylate

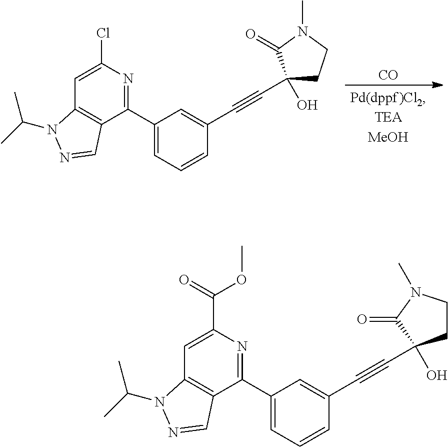 [SMILES: CC(C)n1ncc2c(-c3cccc(C#C[C@]4(O)CCN(C)C4=O)c3)nc(Cl)cc21 ; "CO" ; COC(=O)c1cc2c(cnn2C(C)C)c(-c2cccc(C#C[C@]3(O)CCN(C)C3=O)c2)n1]

Similar to as described in General Procedure O, (3R)-3-(2-[3-[6-chloro-1-(propan-2-yl)-1H-pyrazolo[4,3-c]pyridin-4-yl]phenyl]ethynyl)-3-hydroxy-1-methylpyrrolidin-2-one was reacted with carbon monoxide to give the title compound (200 mg, 63%) as a brown solid. LC-MS (ES, m/z): 433 [M+H]⁺.

### Step 6: Synthesis of 4-(3-[2-[(3R)-3-hydroxy-1-methyl-2-oxopyrrolidin-3-yl]ethynyl]phenyl)-1-(propan-2-yl)-1H-pyrazolo[4,3-c]pyridine-6-carboxamide

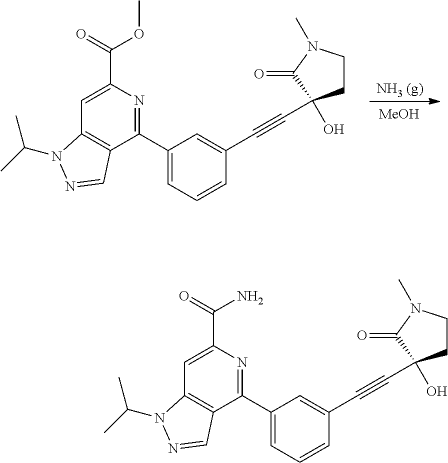

Similar to as described in General Procedure S, methyl 4-(3-[2-[(3R)-3-hydroxy-1-methyl-2-oxopyrrolidin-3-yl]ethynyl]phenyl)-1-(propan-2-yl)-1H-pyrazolo[4,3-c]pyridine-6-carboxylate was reacted with ammonia to give the title compound (40.9 mg, 28%) as an off-white solid. LC-MS (ES, m/z): 418 [M+H]⁺. ¹H NMR (300 MHz, CD₃OD) δ 8.48 (s, 1H), 8.34 (s, 1H), 8.25 (d, J=1.5 Hz, 1H), 8.18-8.15 (m, 1H), 7.65-7.56 (m, 2H), 5.17-5.08 (m, 2H), 3.51-3.46 (m, 2H), 2.93 (s, 3H), 2.65-2.57 (m, 1H), 2.37-2.28 (m, 1H), 1.63 (s, 3H), 1.62 (s, 3H).

### Example J5

### Synthesis of 3-amino-5-fluoro-6-(4-fluoro-3-[2-[(3R)-3-hydroxy-1-methyl-2-oxopyrrolidin-3-yl]ethynyl]phenyl)pyridine-2-carboxamide

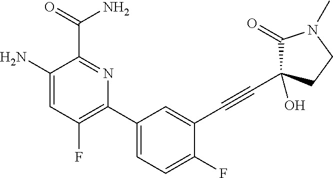 [SMILES: CN1CC[C@@](O)(C#Cc2cc(-c3nc(C(N)=O)c(N)cc3F)ccc2F)C1=O]

### Step 1: Synthesis of methyl 3-amino-5-fluoropyridine-2-carboxylate

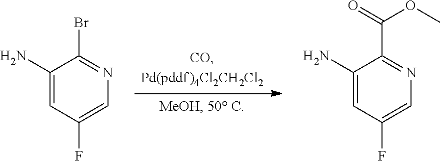

Similar to as described in General Procedure O, 2-bromo-5-fluoropyridin-3-amine was reacted with carbon monoxide to give the title compound (2 g, 56%) as a yellow solid. LC-MS (ES, m/z): 171 [M+H]⁺.

### Step 2: Synthesis of methyl 3-amino-6-bromo-5-fluoropyridine-2-carboxylate

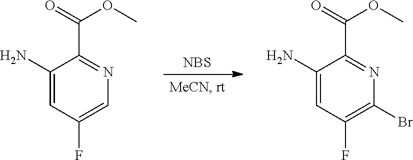

A solution of methyl 3-amino-5-fluoropyridine-2-carboxylate (500 mg, 2.94 mmol, 1.00 equiv) and NBS (620 mg, 3.48 mmol, 1.20 equiv) in acetonitrile (50 mL) was stirred for 12 h at room temperature. The resulting solution was concentrated under vacuum and the residue was purified by a silica gel column eluting with ethyl acetate/petroleum ether (1:10). This resulted in the title compound (0.56 g, 77%) as a white solid. LC-MS (ES, m/z): 249, 251 [M+H]⁺.

### Step 3: Synthesis of 3-amino-6-bromo-5-fluoropyridine-2-carboxamide

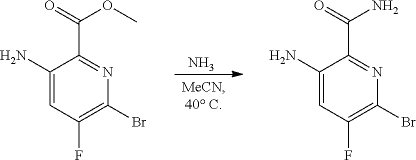

Similar to as described in General Procedure S, methyl 3-amino-6-bromo-5-fluoropyridine-2-carboxylate was reacted with ammonia to give the title compound (0.5 g, 95%) as a yellow solid. LC-MS (ES, m/z): 234, 236 [M+H]⁺.

### Step 4: Synthesis of 3-amino-6-(3-bromo-4-fluorophenyl)-5-fluoropyridine-2-carboxamide

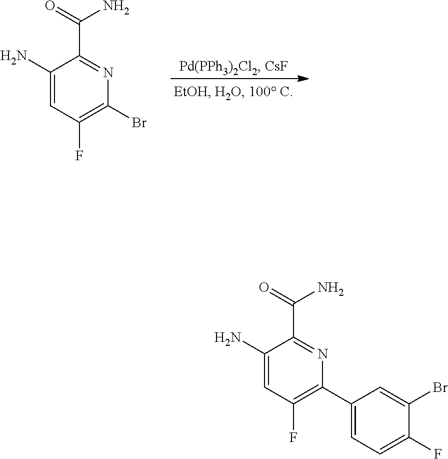

Similar to as described in General Procedure X, 3-amino-6-bromo-5-fluoropyridine-2-carboxamide was reacted with 3-bromo-4-fluorophenylboronic acid to give the title compound (0.16 g, 29%) as a yellow solid. LC-MS (ES, m/z): 328, 330 [M+H]⁺.

### Step 5: Synthesis of 3-amino-5-fluoro-6-(4-fluoro-3-[2-[(3R)-3-hydroxy-1-methyl-2-oxopyrrolidin-3-yl]ethynyl]phenyl)pyridine-2-carboxamide

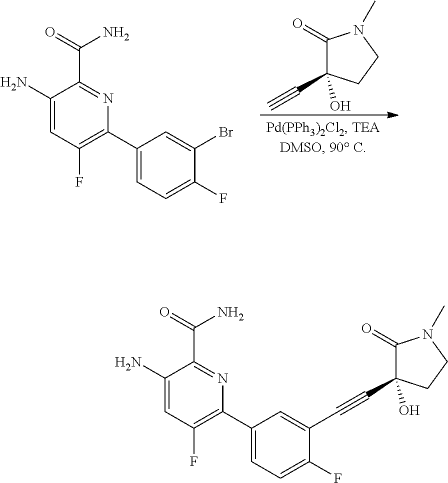

Similar to as described in General Procedure G, 3-amino-6-(3-bromo-4-fluorophenyl)-5-fluoropyridine-2-carboxamide was reacted with (R)-3-ethynyl-3-hydroxy-1-methylpyrrolidin-2-one to give the title compound (0.055 g, 37%) as a white solid. LC-MS (ES, m/z): 387 [M+H]⁺. ¹H NMR (400 MHz, CD₃OD) δ 8.04 (d, J=7.2 Hz, 1H), 7.99-7.96 (m, 1H), 7.24 (t, J=8.8 Hz, 1H), 7.00 (d, J=13.2 Hz, 1H), 3.52-3.47 (m, 2H), 2.95 (s, 3H), 2.66-2.60 (m, 1H), 2.36-2.31 (m, 1H).

### Example K5

### Synthesis of 3-amino-6-(2-fluoro-5-[2-[(3R)-3-hydroxy-1-methyl-2-oxopyrrolidin-3-yl]ethynyl]phenyl)pyrazine-2-carboxamide

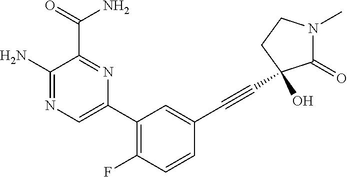 [SMILES: CN1CC[C@@](O)(C#Cc2ccc(F)c(-c3cnc(N)c(C(N)=O)n3)c2)C1=O]

### Step 1: Synthesis of methyl 3-amino-6-(5-bromo-2-fluorophenyl)pyrazine-2-carboxylate

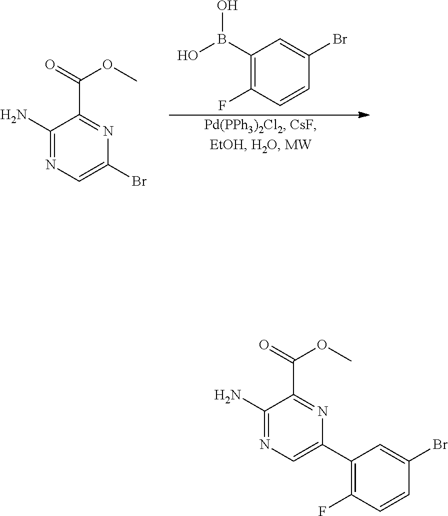

Similar to as described in General Procedure X, methyl 3-amino-6-bromopyrazine-2-carboxylate was reacted with 5-bromo-2-fluorophenylboronic acid to give the title compound (0.26 g, 35%) as a yellow solid. LC-MS (ES, m/z): 326, 328 [M+H]⁺.

### Step 2: Synthesis of 3-amino-6-(5-bromo-2-fluorophenyl)pyrazine-2-carboxamide

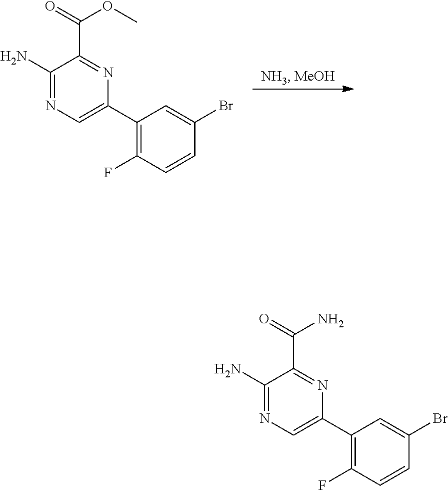

Similar to as described in General Procedure S, methyl 3-amino-6-(5-bromo-2-fluorophenyl)pyrazine-2-carboxylate was reacted with ammonia to give the title compound (120 mg, 63%) as a yellow solid. LC-MS (ES, m/z): 311, 313 [M+H]⁺.

### Step 3: Synthesis of 3-amino-6-(2-fluoro-5-[2-[(3R)-3-hydroxy-1-methyl-2-oxopyrrolidin-3-yl]ethynyl]phenyl)pyrazine-2-carboxamide

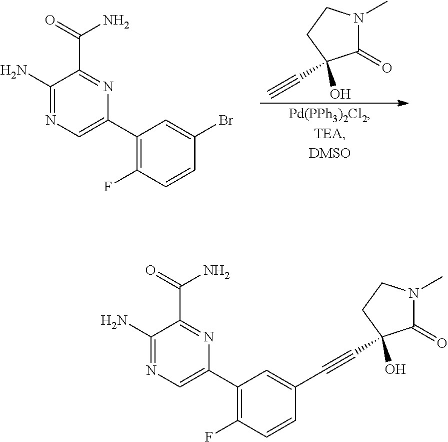

Similar to as described in General Procedure G, 3-amino-6-(5-bromo-2-fluorophenyl)pyrazine-2-carboxamide was reacted with (R)-3-ethynyl-3-hydroxy-1-methylpyrrolidin-2-one to give the title compound (36.5 mg, 31%) as a yellow solid. LC-MS (ES, m/z): 370 [M+H]⁺. ¹H NMR (400 MHz, DMSO-d₆) δ 8.63 (s, 1H), 8.15 (dd, J=7.6, 2.0 Hz, 1H), 7.51-7.48 (m, 1H), 7.38-7.34 (m, 1H), 3.38 (t, J=6.0 Hz, 2H), 2.81 (s, 3H), 2.50-2.46 (m, 1H), 2.22-2.19 (m, 1H).

### Example L5

### Synthesis of 3-amino-6-(2,4-difluoro-5-[2-[(3R)-3-hydroxy-1-methyl-2-oxopyrrolidin-3-yl]ethynyl]phenyl)pyridine-2-carboxamide

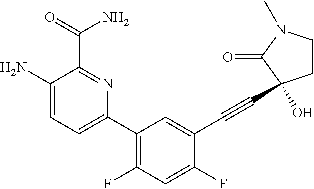 [SMILES: CN1CC[C@@](O)(C#Cc2cc(-c3ccc(N)c(C(N)=O)n3)c(F)cc2F)C1=O]

### Step 1: Synthesis of methyl 3-amino-6-(5-bromo-2,4-difluorophenyl)pyridine-2-carboxylate

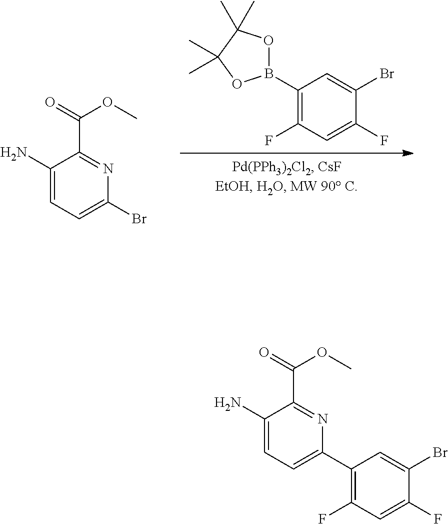

Similar to as described in General Procedure X, methyl 3-amino-6-bromopyridine-2-carboxylate was reacted with 2-(5-bromo-2,4-difluorophenyl)-4,4,5,5-tetramethyl-1,3,2-dioxaborolane to give the title compound (0.08 g, 49%) as a white solid. LC-MS (ES, m/z): 343 [M+H]⁺.

### Step 2: Synthesis of methyl 3-amino-6-(2,4-difluoro-5-[2-[(3R)-3-hydroxy-1-methyl-2-oxopyrrolidin-3-yl]ethynyl]phenyl)pyridine-2-carboxylate

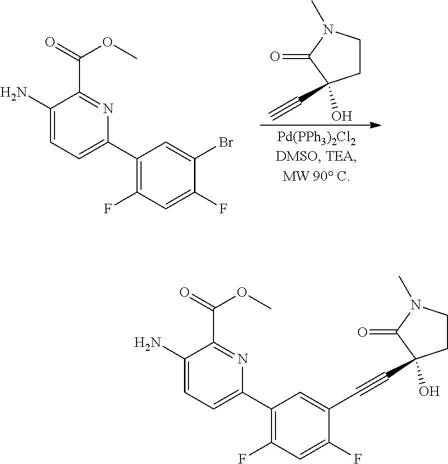

Similar to as described in General Procedure G, methyl 3-amino-6-(5-bromo-2,4-difluorophenyl)pyridine-2-carboxylate was reacted with (R)-3-ethynyl-3-hydroxy-1-methylpyrrolidin-2-one to give the title compound (0.045 g, 55%) as a yellow solid. LC-MS (ES, m/z): 402 [M+H]⁺.

### Step 3: Synthesis of 3-amino-6-(2,4-difluoro-5-[2-[(3R)-3-hydroxy-1-methyl-2-oxopyrrolidin-3-yl]ethynyl]phenyl)pyridine-2-carboxamide

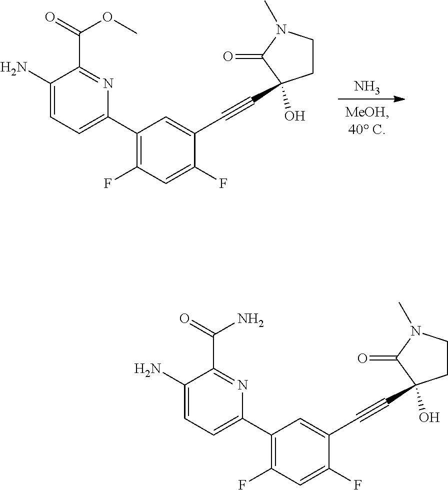

Similar to as described in General Procedure S, methyl 3-amino-6-(2,4-di fluoro-5-[2-[(3R)-3-hydroxy-1-methyl-2-oxopyrrolidin-3-yl]ethynyl]phenyl)pyridine-2-carboxylate was reacted with ammonia to give the title compound (0.0097 g, 20%) as an off-white solid. LC-MS (ES, m/z): [M+H]⁺. ¹H NMR (300 MHz, CD₃OD) δ 8.16 (t, J=6.0 Hz, 1H), 7.68 (d, J=2.7 Hz, 1H), 7.23 (d, J=8.7 Hz, 1H), 7.17-7.09 (m, 1H), 3.51-3.46 (m, 2H), 2.94 (s, 3H), 2.63-2.56 (m, 1H), 2.38-2.29 (m, 1H).

### Example M5

### Synthesis of 6-(4-cyano-3-[2-[(3R)-3-hydroxy-1-methyl-2-oxopyrrolidin-3-yl]ethynyl]phenyl)-3-(ethylamino)pyridine-2-carboxamide

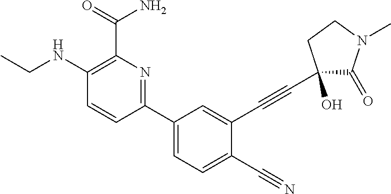 [SMILES: CCNc1ccc(-c2ccc(C#N)c(C#C[C@]3(O)CCN(C)C3=O)c2)nc1C(N)=O]

### Step 1: Synthesis of ethyl 3-(ethylamino)pyridine-2-carboxylate

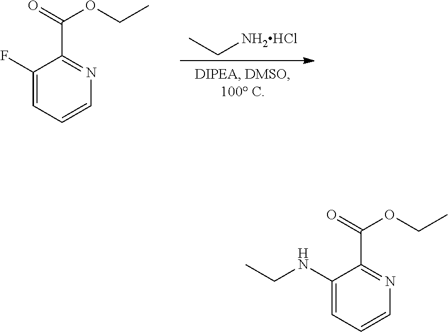

Similar to as described in General Procedure A, ethyl 3-fluoropyridine-2-carboxylate was reacted with ethylamine hydrochloride to give the title compound (537 mg, 92%) as a light brown solid. LC-MS (ES, m/z): 195 [M+H]⁺.

### Step 2: Synthesis of ethyl 6-bromo-3-(ethylamino)pyridine-2-carboxylate

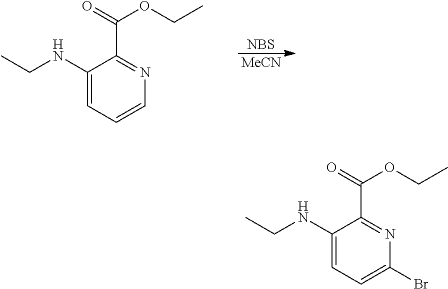

A suspension of ethyl 3-(ethylamino)pyridine-2-carboxylate (650 mg, 3.35 mmol, 1.00 equiv) and N-bromosuccinimide (726 mg, 4.08 mmol, 1.00 equiv) in acetonitrile (15 mL) was stirred for 12 h at room temperature. The resulting solution was concentrated under vacuum and the residue was purified by a silica gel column eluting with ethyl acetate/petroleum ether (1:4). This resulted in the title compound (387 mg, 42%) as a light yellow solid. LC-MS (ES, m/z): 273, 275 [M+H]⁺.

### Step 3: Synthesis of 6-bromo-3-(ethylamino)pyridine-2-carboxamide

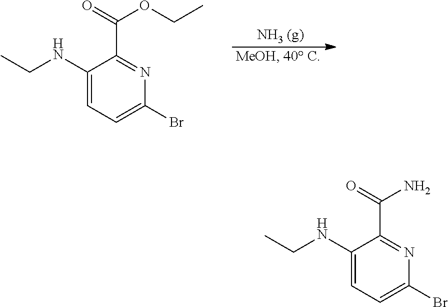

Similar to as described in General Procedure S, ethyl 6-bromo-3-(ethylamino)pyridine-2-carboxylate was reacted with ammonia to give the title compound (359 mg, 100%) as a light brown solid. LC-MS (ES, m/z): 244, 246 [M+H]⁺.

### Step 4: Synthesis of 6-(3-chloro-4-cyanophenyl)-3-(ethylamino)pyridine-2-carboxamide

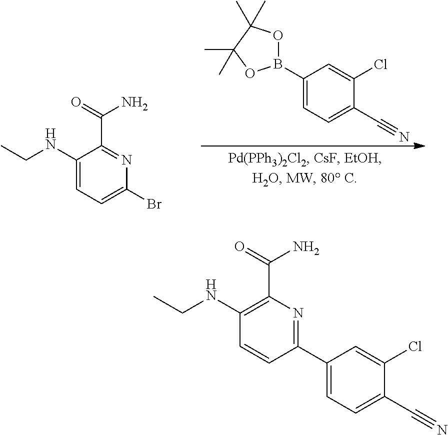 [SMILES: CCNc1ccc(Br)nc1C(N)=O ; CC1(C)OB(c2ccc(C#N)c(Cl)c2)OC1(C)C ; CCNc1ccc(-c2ccc(C#N)c(Cl)c2)nc1C(N)=O]

Similar to as described in General Procedure X, 6-bromo-3-(ethylamino)pyridine-2-carboxamide was reacted with 2-chloro-4-(4,4,5,5-tetramethyl-1,3,2-dioxaborolan-2-yl)benzonitrile to give the title compound (154 mg, 62%) as a light yellow solid. LC-MS (ES, m/z): 301, 303 [M+H]⁺.

### Step 5: Synthesis of 6-(4-cyano-3-[2-[(3R)-3-hydroxy-1-methyl-2-oxopyrrolidin-3-yl]ethynyl]phenyl)-3-(ethylamino)pyridine-2-carboxamide

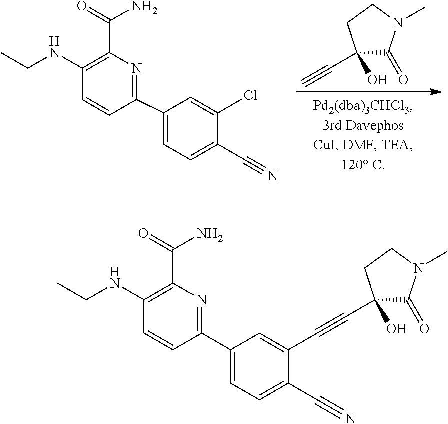

Similar to as described in General Procedure E, 6-(3-chloro-4-cyanophenyl)-3-(ethyl amino)pyridine-2-carboxamide was reacted with (R)-3-ethynyl-3-hydroxy-1-methylpyrrolidin-2-one to give the title compound (2.5 mg, 2%) as a light yellow solid. LC-MS (ES, m/z): 404 [M+H]⁺. ¹H NMR (400 MHz, CD₃OD) δ 8.35 (s, 1H), 8.21 (d, J=8.4 Hz, 1H), 7.99 (d, J=8.8 Hz, 1H), 7.80 (d, J=8.4 Hz, 1H), 7.31 (d, J=8.8 Hz, 1H), 3.63-3.57 (m, 1H), 3.52-3.47 (m, 1H), 2.96 (s, 3H), 2.71-2.65 (m, 1H), 2.40-2.33 (m, 1H), 1.35-1.30 (t, J=7.6 Hz, 3H).

### Example N5

### Synthesis of 5-fluoro-6-(3-[2-[(3R)-3-hydroxy-1-methyl-2-oxopyrrolidin-3-yl]ethynyl]phenyl)-3-[(1,2-oxazol-3-ylmethyl)amino]pyridine-2-carboxamide

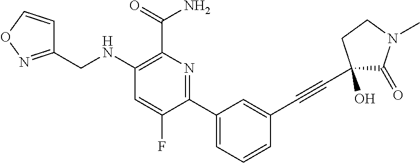 [SMILES: CN1CC[C@@](O)(C#Cc2cccc(-c3nc(C(N)=O)c(NCc4ccon4)cc3F)c2)C1=O]

### Step 1: Synthesis of N-(2-bromo-5-fluoropyridin-3-yl)-1,2-oxazole-3-carboxamide

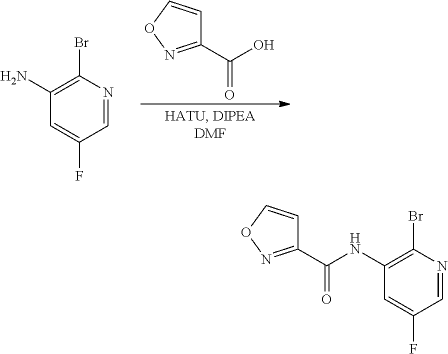 [SMILES: Nc1cc(F)cnc1Br ; O=C(O)c1ccon1 ; O=C(Nc1cc(F)cnc1Br)c1ccon1]

Similar to as described in General Procedure B, 2-bromo-5-fluoropyridin-3-amine was reacted with 1,2-oxazole-3-carboxylic acid to give the title compound (2 g, 70%) as a yellow solid. LC-MS (ES, m/z): 286 [M+H]⁺.

### Step 2: Synthesis of 2-bromo-5-fluoro-N-(1,2-oxazol-3-ylmethyl)pyridin-3-amine

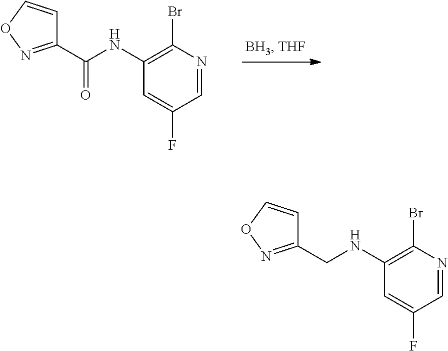 [SMILES: O=C(Nc1cc(F)cnc1Br)c1ccon1 ; Fc1cnc(Br)c(NCc2ccon2)c1]

A solution of N-(2-bromo-5-fluoropyridin-3-yl)-1,2-oxazole-3-carboxamide (2 g, 6.99 mmol, 1.00 equiv), BH₃-THF (35 mL, 365.72 mmol, 1.00 equiv) in THF (100 mL) was stirred overnight at 25° C. The mixture was concentrated under vacuum, mixed with water, and extracted with ethyl acetate. The organic layer was dried over anhydrous sodium sulfate and concentrated under vacuum. The residue was purified by a silica gel column eluting with ethyl acetate/petroleum ether (1:1) to give the title compound (1.3 g, 68%) as a white solid. LC-MS (ES, m/z): 272 [M+H]⁺.

### Step 3: Synthesis of ethyl 5-fluoro-3-[(1,2-oxazol-3-ylmethyl)amino]pyridine-2-carboxylate

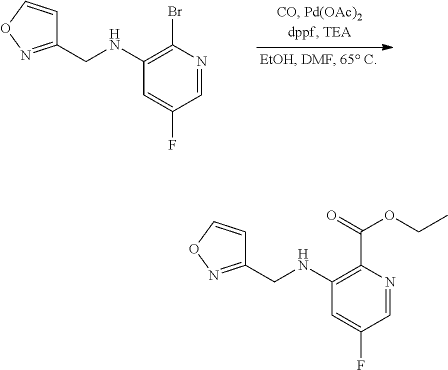 [SMILES: Fc1cnc(Br)c(NCc2ccon2)c1 ; CCOC(=O)c1ncc(F)cc1NCc1ccon1]

Similar to as described in General Procedure O, 2-bromo-5-fluoro-N-(1,2-oxazol-3-ylmethyl)pyridin-3-amine was reacted with carbon monoxide to give the title compound (600 mg, 41%) as a yellow solid. LC-MS (ES, m/z): 266 [M+H]⁺.

### Step 4: Synthesis of ethyl 6-bromo-5-fluoro-3-[(1,2-oxazol-3-ylmethyl)amino]pyridine-2-carboxylate

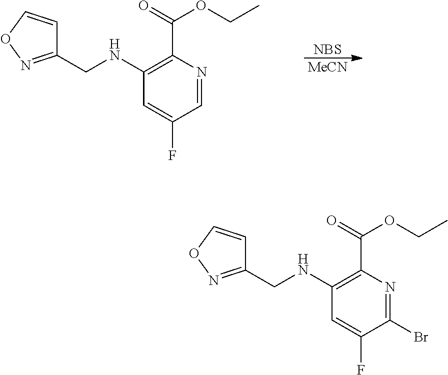 [SMILES: "MeCN" ; CCOC(=O)c1ncc(F)cc1NCc1ccon1 ; "NBS" ; CCOC(=O)c1nc(Br)c(F)cc1NCc1ccon1]

A solution of ethyl 5-fluoro-3-[(1,2-oxazol-3-ylmethyl)amino]pyridine-2-carboxylate (600 mg, 2.26 mmol, 1.00 equiv) and NBS (602 mg, 3.38 mmol, 1.50 equiv) in acetonitrile (100 mL) was stirred for 12 h at 25° C. The resulting mixture was concentrated under vacuum and the residue was purified by a silica gel column eluting with ethyl acetate/petroleum ether (1:1). This resulted in the title compound (280 mg, 36%) as a yellow solid. LC-MS (ES, m/z): 344 [M+H]⁺.

### Step 5: Synthesis of 6-bromo-5-fluoro-3-[(1,2-oxazol-3-ylmethyl)amino]pyridine-2-carboxamide

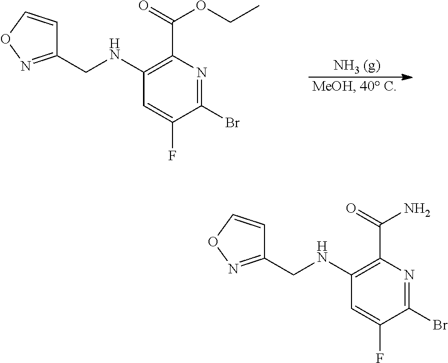 [SMILES: CCOC(=O)c1nc(Br)c(F)cc1NCc1ccon1 ; "MeOH, 40° C." ; "NH3 (g)" ; NC(=O)c1nc(Br)c(F)cc1NCc1ccon1]

Similar to as described in General Procedure S, ethyl 6-bromo-5-fluoro-3-[(1,2-oxazol-3-ylmethyl)amino]pyridine-2-carboxylate was reacted with ammonia to give the title compound (220 mg, 86%) as a yellow solid. LC-MS (ES, m/z): 315 [M+H]⁺.

### Step 6: Synthesis of 5-fluoro-6-(3-[2-[(3R)-3-hydroxy-1-methyl-2-oxopyrrolidin-3-yl]ethynyl]phenyl)-3-[(1,2-oxazol-3-ylmethyl)amino]pyridine-2-carboxamide

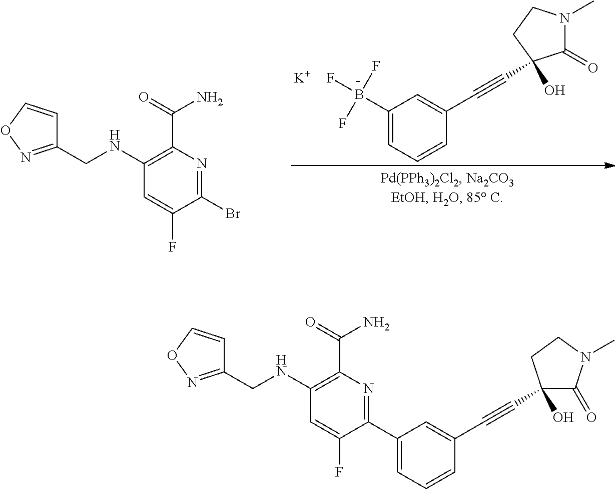

Similar to as described in General Procedure U, 6-bromo-5-fluoro-3-[(1,2-oxazol-3-ylmethyl)amino]pyridine-2-carboxamide was reacted with potassium (R)-trifluoro(3-((3-hydroxy-1-methyl-2-oxopyrrolidin-3-yl)ethynyl)phenyl)borate to give the title compound (48.9 mg, 16%) as a white solid. LC-MS (ES, m/z): 405 [M+H]⁺. ¹H NMR (400 MHz, CD₃OD) δ 8.53 (s, 1H), 7.87-7.83 (m, 1H), 7.81 (d, J=4.8 Hz, 1H), 7.36-7.31 (m, 1H), 7.02 (d, J=14.0 Hz, 1H), 6.42 (s, 1H), 4.52 (s, 2H), 3.42-3.33 (m, 2H), 2.83 (s, 3H), 2.52-2.46 (m, 1H), 2.25-2.18 (m, 1H).

### Example O5

### Synthesis of 3-(3-[2-[(3R)-3-hydroxy-1-methyl-2-oxopyrrolidin-3-yl]ethynyl]phenyl)benzamide

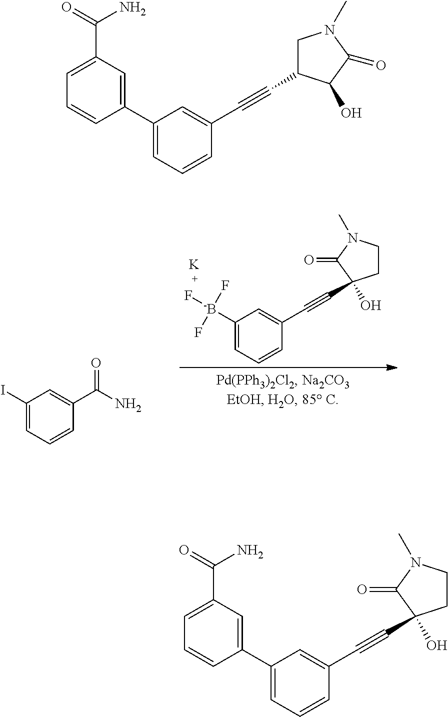

Similar to as described in General Procedure U, 3-iodobenzamide was reacted with potassium (R)-trifluoro(3-((3-hydroxy-1-methyl-2-oxopyrrolidin-3-yl)ethynyl)phenyl)borate to give the title compound (15 mg, 11%) as a white solid. LC-MS (ES, m/z): 335 [M+H]⁺. ¹H NMR (300 MHz, CD₃OD) δ 8.14 (t, J=1.5 Hz, 1H), 7.90 (d, J=0.9 Hz, 1H), 7.88-7.84 (m, 2H), 7.72-7.68 (m, 1H), 7.58 (t, J=7.95 Hz, 1H), 7.49-7.48 (d, J=0.9 Hz, 2H), 3.51-3.46 (m, 2H), 2.94 (s, 3H), 2.64-2.57 (m, 1H), 2.37-2.28 (m, 1H).

### Example P5

### Synthesis of 6-(3-[2-[(3R)-3-hydroxy-1-methyl-2-oxopyrrolidin-3-yl]ethynyl]phenyl)-4-(oxetan-3-yloxy)pyridine-2-carboxamide

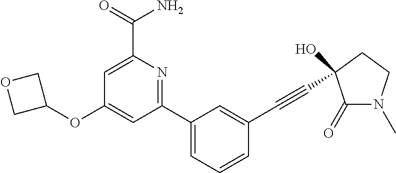 [SMILES: CN1CC[C@@](O)(C#Cc2cccc(-c3cc(OC4COC4)cc(C(N)=O)n3)c2)C1=O]

### Step 1: Synthesis of 2,6-dichloro-4-(oxetan-3-yloxy)pyridine

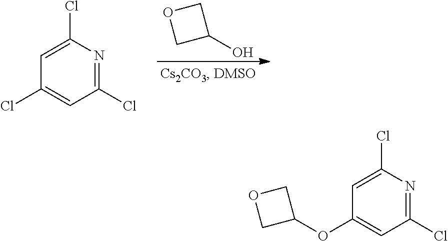 [SMILES: Clc1cc(Cl)nc(Cl)c1 ; OC1COC1 ; Clc1cc(OC2COC2)cc(Cl)n1]

Similar to as described in General Procedure A, 2,4,6-trichloropyridine was reacted with oxetan-3-ol to give the title compound (90 mg, 25%) as a white solid. LC-MS (ES, m/z): 220 [M+H]⁺.

### Step 2: Synthesis of (3R)-3-(2-[3-[6-chloro-4-(oxetan-3-yloxy)pyridin-2-yl]phenyl]ethynyl)-3-hydroxy-1-methyl pyrrolidin-2-one

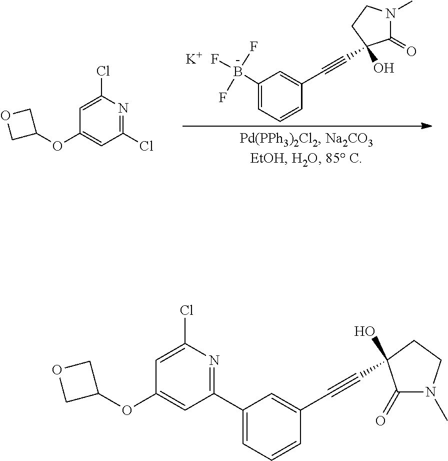

Similar to as described in General Procedure U, 2,6-dichloro-4-(oxetan-3-yloxy)pyridine was reacted with potassium (R)-trifluoro(3-((3-hydroxy-1-methyl-2-oxopyrrolidin-3-yl)ethynyl)phenyl)borate to give the title compound (250 mg, 31%) as a yellow solid. LC-MS (ES, m/z): 399 [M+H]⁺.

### Step 3: Synthesis of methyl 6-(3-[2-[(3R)-3-hydroxy-1-methyl-2-oxopyrrolidin-3-yl]ethynyl]phenyl)-4-(oxetan-3-yloxy)pyridine-2-carboxylate

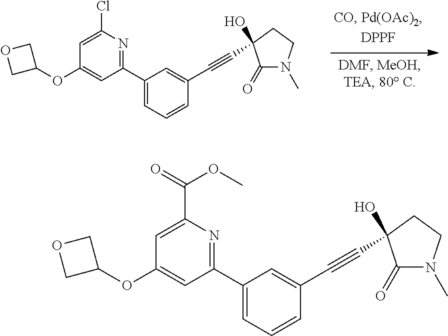 [SMILES: CN1CC[C@@](O)(C#Cc2cccc(-c3cc(OC4COC4)cc(Cl)n3)c2)C1=O ; COC(=O)c1cc(OC2COC2)cc(-c2cccc(C#C[C@]3(O)CCN(C)C3=O)c2)n1]

Similar to as described in General Procedure O, (3R)-3-(2-[3-[6-chloro-4-(oxetan-3-yloxy)pyridin-2-yl]phenyl]ethynyl)-3-hydroxy-1-methyl pyrrolidin-2-one was reacted with carbon monoxide to give the title compound (270 mg, 73%) as a yellow solid. LC-MS (ES, m/z): 423 [M+H]⁺.

### Step 4: Synthesis of

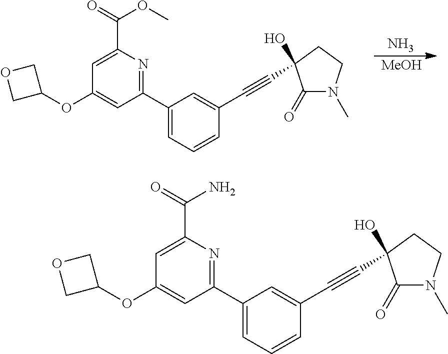

Similar to as described in General Procedure S, methyl 6-(3-[2-[(3R)-3-hydroxy-1-methyl-2-oxopyrrolidin-3-yl]ethynyl]phenyl)-4-(oxetan-3-yloxy)pyridine-2-carboxylate was reacted with ammonia to give the title compound (89.6 mg, 37%) as a white solid. LC-MS (ES, m/z): 408 [M+H]⁺. ¹H NMR (400 MHz, CD₃OD) δ 8.12-8.06 (m, 1H), 7.98-7.95 (m, 1H), 7.41-7.21 (m, 4H), 5.39-5.36 (m, 1H), 4.98-4.95 (m, 2H), 4.62-4.61 (m, 2H), 3.43-3.37 (m, 2H), 2.84 (s, 3H), 2.53-2.48 (m, 1H), 2.26-2.19 (m, 1H).

### Example Q5

### Synthesis of 3-(3-[2-[(3R)-3-hydroxy-1-methyl-2-oxopyrrolidin-3-yl]ethynyl]phenyl)-5-methylbenzamide

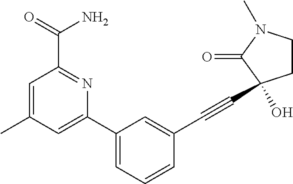 [SMILES: Cc1cc(C(N)=O)nc(-c2cccc(C#C[C@]3(O)CCN(C)C3=O)c2)c1]

### Step 1: Synthesis of (3R)-3-[2-[3-(6-bromo-4-methyl pyridin-2-yl)phenyl]ethynyl]-3-hydroxy-1-methylpyrrolidin-2-one

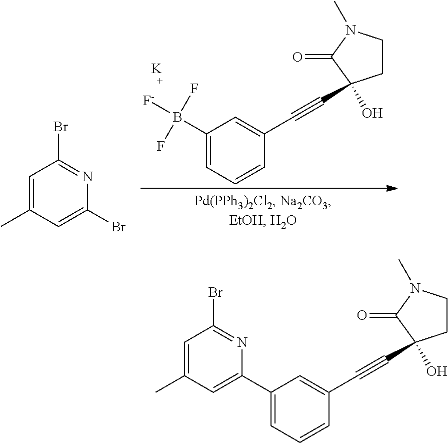

Similar to as described in General Procedure U, 2,6-dibromo-4-methylpyridine was reacted with potassium (R)-trifluoro(3-((3-hydroxy-1-methyl-2-oxopyrrolidin-3-yl)ethynyl)phenyl)borate to give the title compound (200 mg, 26%) as a light brown solid. LC-MS (ES, m/z): 385, 387 [M+H]⁺.

### Step 2: Synthesis of methyl 6-(3-[2-[(3R)-3-hydroxy-1-methyl-2-oxopyrrolidin-3-yl]ethynyl]phenyl)-4-methyl pyridine-2-carboxylate

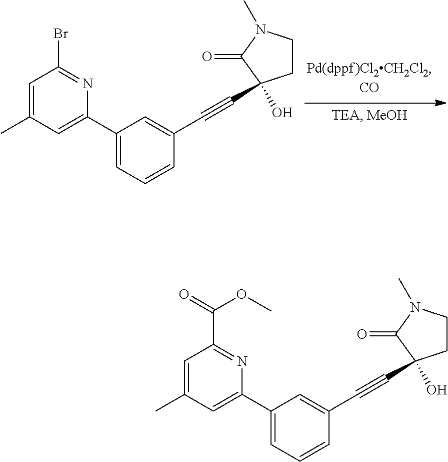 [SMILES: Cc1cc(Br)nc(-c2cccc(C#C[C@]3(O)CCN(C)C3=O)c2)c1 ; COC(=O)c1cc(C)cc(-c2cccc(C#C[C@]3(O)CCN(C)C3=O)c2)n1]

Similar to as described in General Procedure O, (3R)-3-[2-[3-(6-bromo-4-methyl pyridin-2-yl)phenyl]ethynyl]-3-hydroxy-1-methylpyrrolidin-2-one was reacted with carbon monoxide to give the title compound (280 mg, 74%) as a light brown solid. LC-MS (ES, m/z): 365 [M+H]⁺.

### Step 3: Synthesis of 3-(3-[2-[(3R)-3-hydroxy-1-methyl-2-oxopyrrolidin-3-yl]ethynyl]phenyl)-5-methylbenzamide

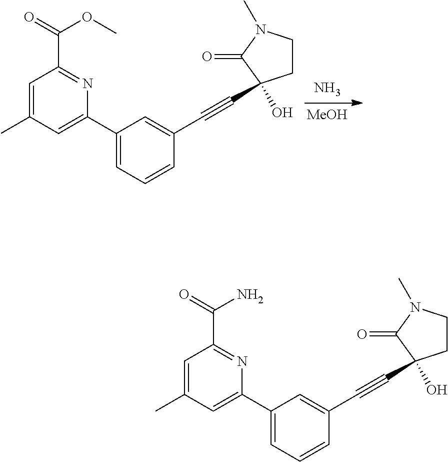

Similar to as described in General Procedure S, methyl 3-(3-[2-[(3R)-3-hydroxy-1-methyl-2-oxopyrrolidin-3-yl]ethynyl]phenyl)-5-methylbenzoate was reacted with ammonia to give the title compound (56.7 mg, 21%) as a white solid. LC-MS (ES, m/z): 350 [M+H]⁺. ¹H NMR (400 MHz, CD₃OD) δ 8.29 (s, 1H), 8.18 (d, J=7.6 Hz, 1H), 7.95 (s, 2H), 7.56-7.49 (m, 2H), 3.55-3.47 (m, 2H), 2.95 (s, 3H), 2.70-2.59 (m, 1H), 2.54 (s, 3H), 2.37-2.30 (m, 1H).

### Example R5

### Synthesis of 4-amino-6-(3-[2-[(3R)-3-hydroxy-1-methyl-2-oxopyrrolidin-3-yl]ethynyl]phenyl)pyridine-2-carboxamide

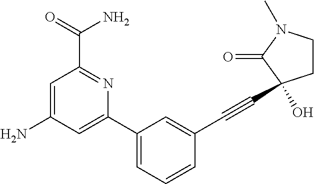 [SMILES: CN1CC[C@@](O)(C#Cc2cccc(-c3cc(N)cc(C(N)=O)n3)c2)C1=O]

### Step 1: Synthesis of (3R)-3-[2-[3-(4-amino-6-chloropyridin-2-yl)phenyl]ethynyl]-3-hydroxy-1-methylpyrrolidin-2-one

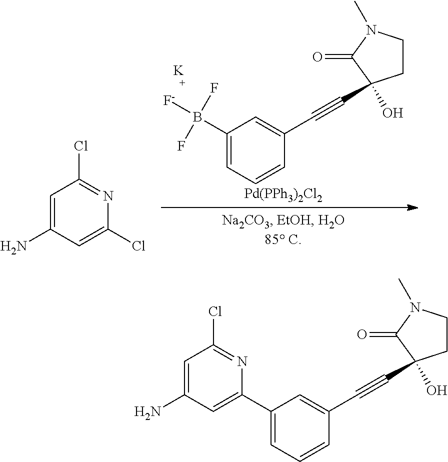

Similar to as described in General Procedure U, 2,6-dichloropyridin-4-amine was reacted with potassium (R)-trifluoro(3-((3-hydroxy-1-methyl-2-oxopyrrolidin-3-yl)ethynyl)phenyl)borate to give the title compound (80 mg, 6%) as a yellow solid. LC-MS (ES, m/z): 342 [M+H]⁺.

### Step 2: Synthesis of 4-amino-6-(3-[2-[(3R)-3-hydroxy-1-methyl-2-oxopyrrolidin-3-yl]ethynyl]phenyl)pyridine-2-carboxamide

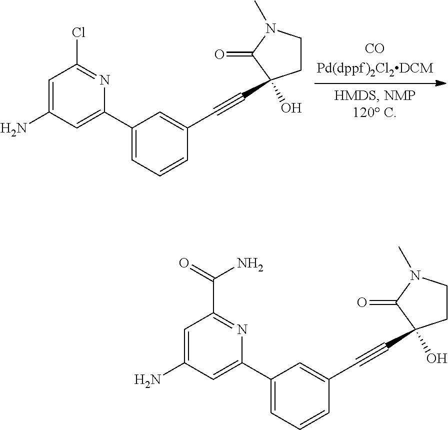 [SMILES: CN1CC[C@@](O)(C#Cc2cccc(-c3cc(N)cc(Cl)n3)c2)C1=O ; CN1CC[C@@](O)(C#Cc2cccc(-c3cc(N)cc(C(N)=O)n3)c2)C1=O]

Similar to as described in General Procedure P, (3R)-3-[2-[3-(4-amino-6-chloropyridin-2-yl)phenyl]ethynyl]-3-hydroxy-1-methylpyrrolidin-2-one was reacted with hexamethyldisilazane to give the title compound (15.7 mg, 16%) as a white solid. LC-MS (ES, m/z): 351 [M+H]⁺. ¹H NMR (300 MHz, CD₃OD, ppm) δ 8.16 (s, 1H), 8.05 (d, J=7.5 Hz, 1H), 7.53-7.44 (m, 2H), 7.32 (d, J=2.1 Hz, 1H), 7.18 (d, J=2.1 Hz, 1H), 3.54-3.49 (m, 2H), 2.96 (s, 1H), 2.67-2.59 (m, 1H), 2.35-2.30 (m, 1H).

### Example G02925803

### Synthesis of 6-(3-[2-[(3R)-3-hydroxy-1-methyl-2-oxopyrrolidin-3-yl]ethynyl]phenyl)-4-(1-methyl-1H-pyrazol-5-yl)pyridine-2-carboxamide

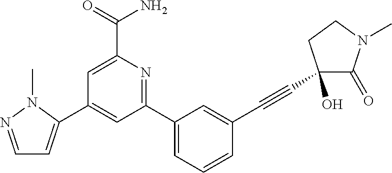 [SMILES: CN1CC[C@@](O)(C#Cc2cccc(-c3cc(-c4ccnn4C)cc(C(N)=O)n3)c2)C1=O]

### Step 1: Synthesis of ethyl 6-(3-[2-[(3R)-3-hydroxy-1-methyl-2-oxopyrrolidin-3-yl]ethynyl]phenyl)-4-(1-methyl-1H-pyrazol-5-yl)pyridine-2-carboxylate

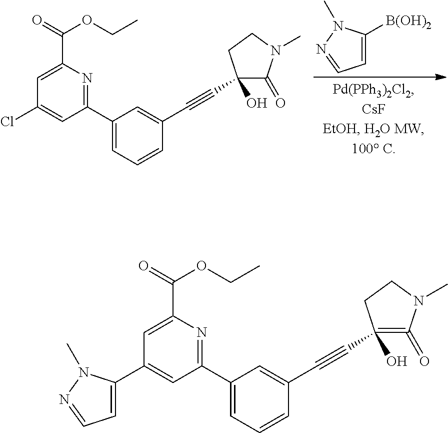

Similar to as described in General Procedure X, ethyl 4-chloro-6-(3-[2-[(3R)-3-hydroxy-1-methyl-2-oxopyrrolidin-3-yl]ethynyl]phenyl)pyridine-2-carboxylate was reacted with 1-methyl-1H-pyrazol-5-ylboronic acid to give the title compound (115 mg, 52%) as a brown solid. LC-MS (ES, m/z): 445 [M+H]⁺.

### Step 2: Synthesis of 6-(3-[2-[(3R)-3-hydroxy-1-methyl-2-oxopyrrolidin-3-yl]ethynyl]phenyl)-4-(1-methyl-1H-pyrazol-5-yl)pyridine-2-carboxamide

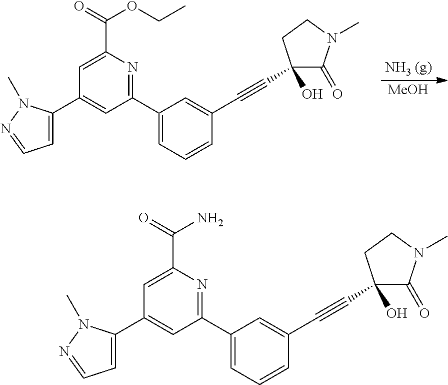 [SMILES: "MeOH" ; CCOC(=O)c1cc(-c2ccnn2C)cc(-c2cccc(C#C[C@]3(O)CCN(C)C3=O)c2)n1 ; CN1CC[C@@](O)(C#Cc2cccc(-c3cc(-c4ccnn4C)cc(C(N)=O)n3)c2)C1=O]

Similar to as described in General Procedure S, ethyl 6-(3-[2-[(3R)-3-hydroxy-1-methyl-2-oxopyrrolidin-3-yl]ethynyl]phenyl)-4-(1-methyl-1H-pyrazol-5-yl)pyridine-2-carboxylate was reacted with ammonia to give the title compound (26.3 mg, 28%) as a white solid. LC-MS (ES, m/z): 416 [M+H]⁺. ¹H NMR (300 MHz, CD₃OD) δ 8.36 (s, 1H), 8.26-8.19 (m, 3H), 7.59-7.49 (m, 3H), 7.70 (d, J=2.1 Hz, 1H), 4.03 (s, 3H), 3.51-3.46 (m, 2H), 2.92 (s, 3H), 2.64-2.56 (m, 1H), 2.36-2.27 (m, 1H).

### Example S5

### Synthesis of 6-(3-[2-[(3R)-3-hydroxy-1-methyl-2-oxopyrrolidin-3-yl]ethynyl]phenyl)-4-(5-methyl-1H-pyrazol-1-yl)pyridine-2-carboxamide

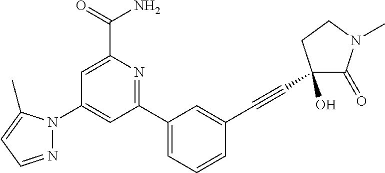 [SMILES: Cc1ccnn1-c1cc(C(N)=O)nc(-c2cccc(C#C[C@]3(O)CCN(C)C3=O)c2)c1]

### Step 1: Synthesis of 2,6-dichloro-4-hydrazinylpyridine

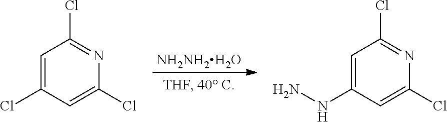

Similar to as described in General Procedure A, 2,4,6-trichloropyridine was reacted with hydrazine hydrate to give the title compound (330 mg, 34%) as a white solid. LC-MS (ES, m/z): 178 [M+H]⁺.

### Step 2: Synthesis of 2,6-dichloro-4-(5-methyl-1H-pyrazol-1-yl)pyridine

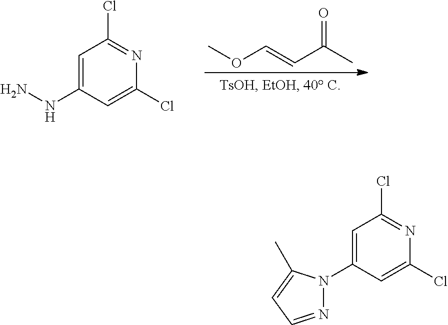

A solution of 2,6-dichloro-4-hydrazinylpyridine (610 mg, 3.43 mmol, 1.00 equiv), (3E)-4-methoxybut-3-en-2-one (342 mg, 3.42 mmol, 1.00 equiv), and p-toluenesulfonic acid (59 mg, 0.34 mmol, 0.10 equiv) in ethanol (30 mL) was stirred for 18 h at 40° C. The resulting solution was concentrated under vacuum and the residue was purified by a silica gel column eluting with ethyl acetate/petroleum ether (1:5). This resulted in the title compound (300 mg, 38%) as a white solid. LC-MS (ES, m/z): 228 [M+H]⁺.

### Step 3: Synthesis of (3R)-3-(2-[3-[6-chloro-4-(5-methyl-1H-pyrazol-1-yl)pyridin-2-yl]phenyl]ethynyl)-3-hydroxy-1-methyl pyrrolidin-2-one

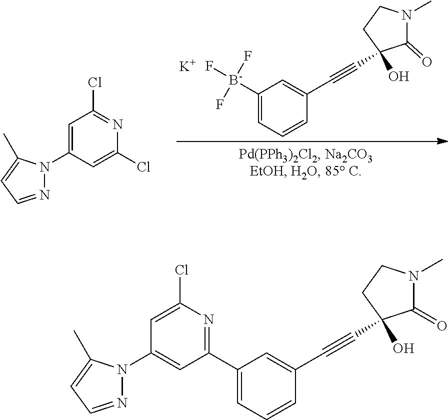 [SMILES: Cc1ccnn1-c1cc(Cl)nc(Cl)c1 ; [K+] ; CN1CC[C@@](O)(C#Cc2cccc([B-](F)(F)F)c2)C1=O ; Cc1ccnn1-c1cc(Cl)nc(-c2cccc(C#C[C@]3(O)CCN(C)C3=O)c2)c1]

Similar to as described in General Procedure U, 2,6-dichloro-4-(5-methyl-1H-pyrazol-1-yl)pyridine was reacted with potassium (R)-trifluoro(3-((3-hydroxy-1-methyl-2-oxopyrrolidin-3-yl)ethynyl)phenyl)borate to give the title compound (200 mg, 32%) as a brown solid. LC-MS (ES, m/z): 407 [M+H]⁺.

### Step 4: Synthesis of methyl 6-(3-[2-[(3R)-3-hydroxy-1-methyl-2-oxopyrrolidin-3-yl]ethynyl]phenyl)-4-(5-methyl-1H-pyrazol-1-yl)pyridine-2-carboxylate

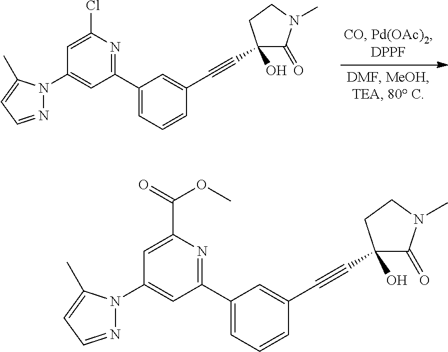 [SMILES: Cc1ccnn1-c1cc(Cl)nc(-c2cccc(C#C[C@]3(O)CCN(C)C3=O)c2)c1 ; COC(=O)c1cc(-n2nccc2C)cc(-c2cccc(C#C[C@]3(O)CCN(C)C3=O)c2)n1]

Similar to as described in General Procedure O, (3R)-3-(2-[3-[6-chloro-4-(5-methyl-1H-pyrazol-1-yl)pyridin-2-yl]phenyl]ethynyl)-3-hydroxy-1-methylpyrrolidin-2-one was reacted with carbon monoxide to give the title compound (150 mg, 71%) as a yellow solid. LC-MS (ES, m/z): 431 [M+H]⁺.

### Step 5: Synthesis of 6-(3-[2-[(3R)-3-hydroxy-1-methyl-2-oxopyrrolidin-3-yl]ethynyl]phenyl)-4-(5-methyl-1H-pyrazol-1-yl)pyridine-2-carboxamide

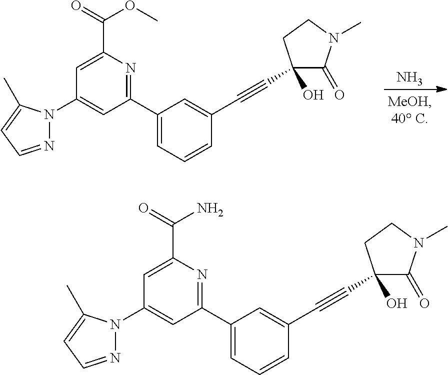

Similar to as described in General Procedure S, methyl 6-(3-[2-[(3R)-3-hydroxy-1-methyl-2-oxopyrrolidin-3-yl]ethynyl]phenyl)-4-(5-methyl-1H-pyrazol-1-yl)pyridine-2-carboxylate was reacted with ammonia to give the title compound (17.9 mg, 12%) as a white solid. LC-MS (ES, m/z): 416 [M+H]⁺. ¹H NMR (400 MHz, CD₃OD) δ 8.26 (s, 1H), 8.19-8.12 (m, 3H), 7.61 (s, 1H), 7.50-7.41 (m, 2H), 6.31 (s, 1H), 3.44-3.35 (m, 2H), 2.84 (s, 3H), 2.55-2.49 (m, 4H), 2.26-2.19 (m, 1H).

### Example T5

### Synthesis of 6-(3-[2-[(3R)-3-hydroxy-1-methyl-2-oxopyrrolidin-3-yl]ethynyl]phenyl)-4-(6-methylpyridin-3-yl)pyridine-2-carboxamide

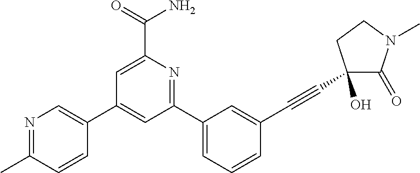

### Step 1: Synthesis of ethyl 6-(3-[2-[(3R)-3-hydroxy-1-methyl-2-oxopyrrolidin-3-yl]ethynyl]phenyl)-4-(6-methylpyridin-3-yl)pyridine-2-carboxylate

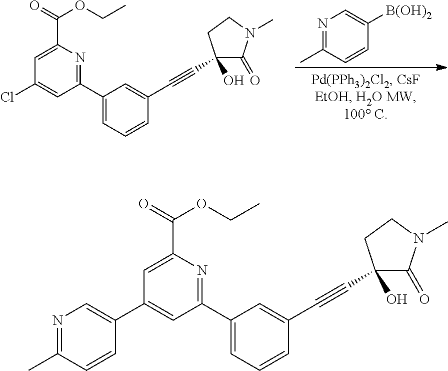

Similar to as described in General Procedure X, ethyl 4-chloro-6-(3-[2-[(3R)-3-hydroxy-1-methyl-2-oxopyrrolidin-3-yl]ethynyl]phenyl)pyridine-2-carboxylate was reacted with (6-methylpyridin-3-yl)boronic acid to give the title compound (100 mg, 42%) as a brown solid. LC-MS (ES, m/z): 456 [M+H]⁺.

### Step 2: Synthesis of 6-(3-[2-[(3R)-3-hydroxy-1-methyl-2-oxopyrrolidin-3-yl]ethynyl]phenyl)-4-(6-methylpyridin-3-yl)pyridine-2-carboxamide

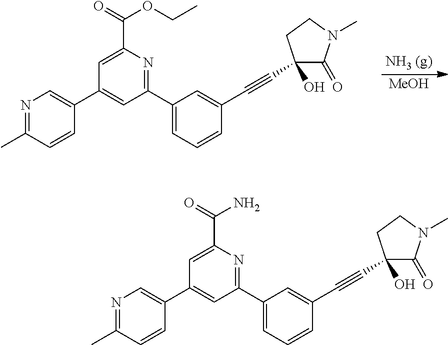

Similar to as described in General Procedure S, ethyl 6-(3-[2-[(3R)-3-hydroxy-1-methyl-2-oxopyrrolidin-3-yl]ethynyl]phenyl)-4-(6-methylpyridin-3-yl)pyridine-2-carboxylate was reacted with ammonia to give the title compound (46 mg, 55%) as a white solid. LC-MS (ES, m/z): 427 [M+H]⁺. ¹H NMR (300 MHz, CD₃OD) δ 8.96 (d, J=2.1 Hz, 1H), 8.43-8.40 (m, 3H), 8.40-8.26 (m, 2H), 7.62-7.52 (m, 3H), 3.55-3.50 (m, 2H), 3.51-3.46 (m, 2H), 2.97 (s, 3H), 2.69-2.61 (m, 4H), 2.40.-2.31 (m, 1H).

### Example U5

### Synthesis of 6-(3-[2-[(3R)-3-hydroxy-1-methyl-2-oxopyrrolidin-3-yl]ethynyl]phenyl)-4-(pyrazin-2-yl)pyridine-2-carboxamide

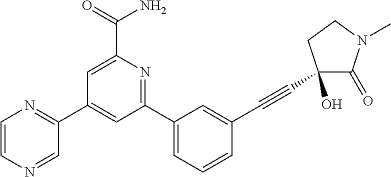 [SMILES: CN1CC[C@@](O)(C#Cc2cccc(-c3cc(-c4cnccn4)cc(C(N)=O)n3)c2)C1=O]

### Step 1: Synthesis of ethyl 6-(3-[2-[(3R)-3-hydroxy-1-methyl-2-oxopyrrolidin-3-yl]ethynyl]phenyl)-4-(pyrazin-2-yl)pyridine-2-carboxylate

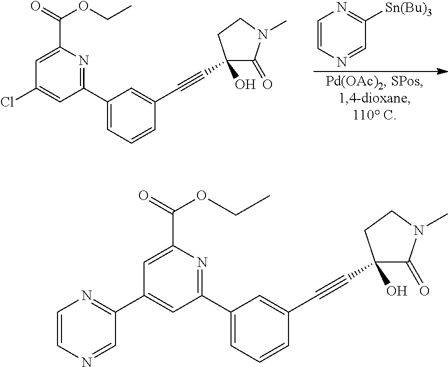

A mixture of ethyl 4-chloro-6-(3-[2-[(3R)-3-hydroxy-1-methyl-2-oxopyrrolidin-3-yl]ethynyl]phenyl)pyridine-2-carboxylate (100.00 mg, 0.25 mmol, 1.00 equiv), 2-(tributylstannyl)pyrazine (111.06 mg, 0.30 mmol, 1.20 equiv), Pd(OAc)₂ (5.63 mg, 0.03 mmol, 0.10 equiv), and SPhos (20.59 mg, 0.05 mmol, 0.20 equiv) in 1,4-dioxane (3 mL) was irradiated with microwave for 2 h at 110° C. The reaction was quenched by potassium fluoride solution. The precipitate was filtered off and the filtrate was extracted with ethyl acetate and washed with brine. The organic layers were combined, dried over anhydrous sodium sulfate, and concentrated under vacuum. The residue was purified by a silica gel column chromatography eluting with ethyl acetate/petroleum ether (2:1) to give the title compound (70 mg, 63%) as a light yellow solid. LC-MS (ES, m/z): 443 [M+H]⁺.

### Step 2: Synthesis of 6-(3-[2-[(3R)-3-hydroxy-1-methyl-2-oxopyrrolidin-3-yl]ethynyl]phenyl)-4-(pyrazin-2-yl)pyridine-2-carboxamide

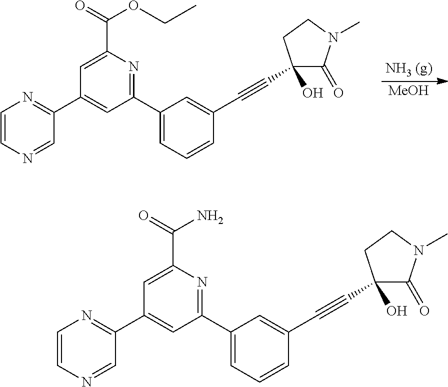 [SMILES: "MeOH" ; CCOC(=O)c1cc(-c2cnccn2)cc(-c2cccc(C#C[C@]3(O)CCN(C)C3=O)c2)n1 ; CN1CC[C@@](O)(C#Cc2cccc(-c3cc(-c4cnccn4)cc(C(N)=O)n3)c2)C1=O]

Similar to as described in General Procedure S, ethyl 6-(3-[2-[(3R)-3-hydroxy-1-methyl-2-oxopyrrolidin-3-yl]ethynyl]phenyl)-4-(pyrazin-2-yl)pyridine-2-carboxylate was reacted with ammonia to give the title compound (29.7 mg, 63%) as a white solid. LC-MS (ES, m/z): 414 [M+H]⁺. ¹H NMR (300 MHz, CD₃OD) δ 9.43 (d, J=1.5 Hz, 1H), 9.84-9.83 (m, 1H), 8.81 (s, 2H), 8.73-8.72 (m, 1H), 8.42 (d, J=1.2 Hz, 1H), 8.32 (d, J=7.5 Hz, 1H), 7.60-7.52 (m, 2H), 3.51-3.49 (m, 2H), 2.95 (s, 3H), 2.63-2.61 (m, 1H), 2.36-2.32 (m, 1H).

### Example V5

### Synthesis of 6-(3-[2-[(3R)-3-hydroxy-1-methyl-2-oxopyrrolidin-3-yl]ethynyl]phenyl)-3-[(2-methoxyethyl)amino]pyridine-2-carboxamide

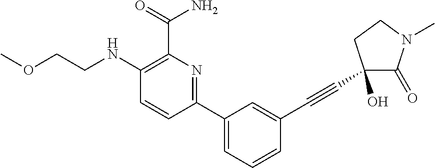

### Step 1: Synthesis of ethyl 3-fluoropyridine-2-carboxylate

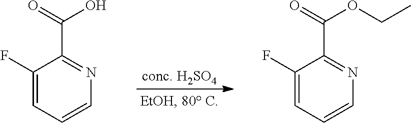

To a solution of 3-fluoropyridine-2-carboxylic acid (1 g, 7.09 mmol, 1.00 equiv) in ethanol (30 mL) was added sulfuric acid (0.2 mL, 3.75 mmol, 0.50 equiv). The resulting solution was stirred at 80° C. for 3 hours and concentrated under vacuum. The residue was poured into water, extracted with dichloromethane, dried with sodium sulfate, and concentrated under vacuum. The residue was purified by a silica gel column eluting with ethyl acetate/petroleum ether (1:10) to give the title compound (0.66 g, 55%) as colorless oil. LC-MS (ES, m/z): 170 [M+H]⁺.

### Step 2: Synthesis of ethyl 3-[(2-methoxyethyl)amino]pyridine-2-carboxylate

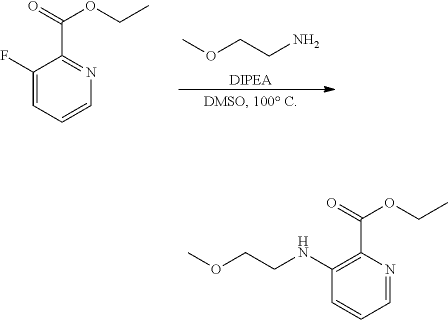

Similar to as described in General Procedure A, ethyl 3-fluoropyridine-2-carboxylate was reacted with 2-methoxyethan-1-amine to give the title compound (1.89 g, 95%) as light yellow oil. LC-MS (ES, m/z): 225 [M+H]⁺.

### Step 3: Synthesis of ethyl 6-bromo-3-[(2-methoxyethyl)amino]pyridine-2-carboxylate

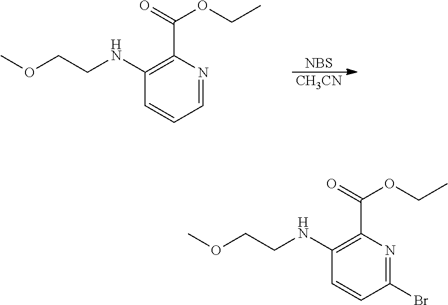 [SMILES: CCOC(=O)c1ncccc1NCCOC ; "CH3CN" ; "NBS" ; CCOC(=O)c1nc(Br)ccc1NCCOC]

A solution of ethyl 3-[(2-methoxyethyl)amino]pyridine-2-carboxylate (1.00 g, 4.46 mmol, 1.00 equiv) and NBS (950 mg, 5.34 mmol, 1.20 equiv) in acetonitrile (40 mL) was stirred for at 25° C. 3 hours. The resulting solution was concentrated under vacuum and the residue was purified by a silica gel column chromatography eluting with ethyl acetate/petroleum ether (1:5) to give the title compound (1.332 g, 99%) as light yellow oil. LC-MS (ES, m/z): 303 [M+H]⁺.

### Step 4: Synthesis of 6-bromo-3-[(2-methoxyethyl)amino]pyridine-2-carboxamide

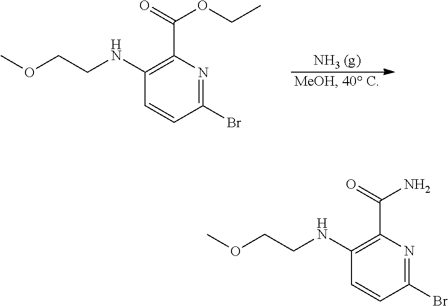 [SMILES: CCOC(=O)c1nc(Br)ccc1NCCOC ; "MeOH, 40° C." ; "NH3 (g)" ; COCCNc1ccc(Br)nc1C(N)=O]

Similar to as described in General Procedure S, ethyl 6-bromo-3-[(2-methoxyethyl)amino]pyridine-2-carboxylate was reacted with ammonia to give the title compound (769 mg, 85%) as a light yellow solid. LC-MS (ES, m/z): 274 [M+H]⁺.

### Step 5: Synthesis of 6-(3-[2-[(3R)-3-hydroxy-1-methyl-2-oxopyrrolidin-3-yl]ethynyl]phenyl)-3-[(2-methoxyethyl)amino]pyridine-2-carboxamide

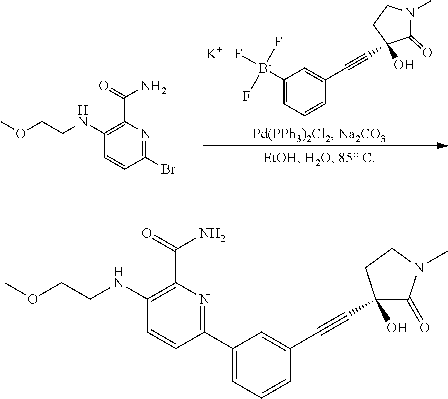

Similar to as described in General Procedure U, 6-bromo-3-[(2-hydroxyethyl)amino]pyridine-2-carboxamide was reacted with potassium (R)-trifluoro(3-((3-hydroxy-1-methyl-2-oxopyrrolidin-3-yl)ethynyl)phenyl)borate to give the title compound (412 mg, 52%) as a light yellow solid. LC-MS (ES, m/z): 409 [M+H]⁺. ¹H NMR (300 Mhz, CD₃OD) δ 8.08 (s, 1H), 8.00-7.97 (m, 1H), 7.87 (d, J=8.7 Hz, 1H), 7.43-7.39 (m, 2H), 7.33 (d, J=9.0 Hz, 1H), 3.87-3.86 (m, 2H), 3.66-3.30 (m, 7H), 3.30 (s, 1H), 2.92 (s, 3H), 2.69-2.55 (m, 1H), 2.35-2.26 (m, 1H).

### Example W5

### Synthesis of 6-(3-[2-[(3R)-3-hydroxy-1-methyl-2-oxopyrrolidin-3-yl]ethynyl]phenyl)-4-(pyrimidin-2-yl)pyridine-2-carboxamide

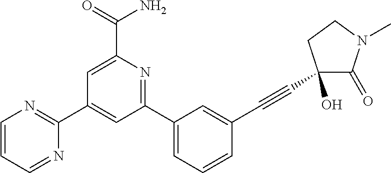 [SMILES: CN1CC[C@@](O)(C#Cc2cccc(-c3cc(-c4ncccn4)cc(C(N)=O)n3)c2)C1=O]

### Step 1: Synthesis of ethyl 4-chloro-6-(3-[2-[(3R)-3-hydroxy-1-methyl-2-oxopyrrolidin-3-yl]ethynyl]phenyl)pyridine-2-carboxylate

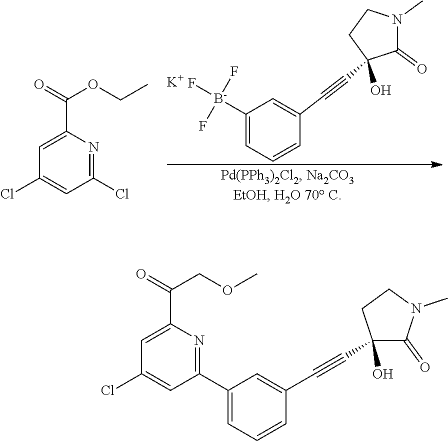 [SMILES: CCOC(=O)c1cc(Cl)cc(Cl)n1 ; [K+] ; CN1CC[C@@](O)(C#Cc2cccc([B-](F)(F)F)c2)C1=O ; COCC(=O)c1cc(Cl)cc(-c2cccc(C#C[C@]3(O)CCN(C)C3=O)c2)n1]

Similar to as described in General Procedure U, methyl 4,6-di chloropyridine-2-carboxylate was reacted with potassium (R)-trifluoro(3-((3-hydroxy-1-methyl-2-oxopyrrolidin-3-yl)ethynyl)phenyl)borate to give the title compound (400 mg, 21%) as a light brown solid. LC-MS (ES, m/z): 399 [M+H]⁺.

### Step 2: Synthesis of ethyl 6-(3-[2-[(3R)-3-hydroxy-1-methyl-2-oxopyrrolidin-3-yl]ethynyl]phenyl)-4-(pyrimidin-2-yl)pyridine-2-carboxylate

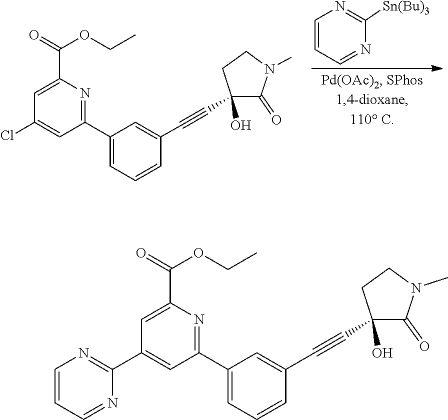

A solution of ethyl 4-chloro-6-(3-[2-[(3R)-3-hydroxy-1-methyl-2-oxopyrrolidin-3-yl]ethynyl]phenyl)pyridine-2-carboxylate (300.00 mg, 0.75 mmol, 1.00 equiv), 2-(tributylstannyl)pyrimidine (333.18 mg, 0.90 mmol, 1.20 equiv), Pd(OAc)₂ (16.89 mg, 0.08 mmol, 0.10 equiv), and SPhos (61.76 mg, 0.15 mmol, 0.20 equiv) in 1,4-dioxane (3 mL) was stirred for 2 hours at 110° C. The reaction was then quenched by aqueous potassium fluoride solution. The precipitate was filtered off and the filtrate was extracted with ethyl acetate and washed with brine. The organic layers were combined, dried over anhydrous sodium sulfate, and concentrated under vacuum. The residue was purified by a silica gel column eluting with ethyl acetate/petroleum ether (1:1). This resulted in the title compound (120 mg, 36%) as a light yellow solid. LC-MS (ES, m/z): 443 [M+H]⁺.

### Step 3: Synthesis of 6-(3-[2-[(3R)-3-hydroxy-1-methyl-2-oxopyrrolidin-3-yl]ethynyl]phenyl)-4-(pyrimidin-2-yl)pyridine-2-carboxamide

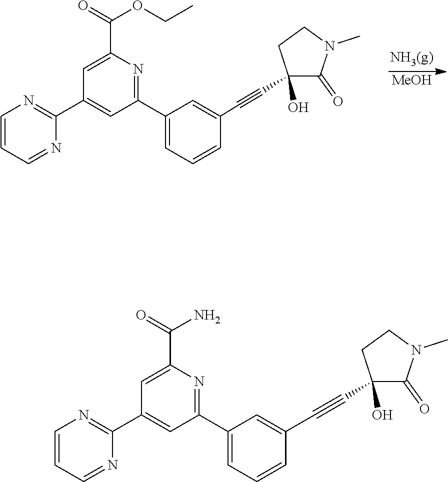 [SMILES: "MeOH" ; CCOC(=O)c1cc(-c2ncccn2)cc(-c2cccc(C#C[C@]3(O)CCN(C)C3=O)c2)n1 ; CN1CC[C@@](O)(C#Cc2cccc(-c3cc(-c4ncccn4)cc(C(N)=O)n3)c2)C1=O]

Similar to as described in General Procedure S, ethyl 6-(3-[2-[(3R)-3-hydroxy-1-methyl-2-oxopyrrolidin-3-yl]ethynyl]phenyl)-4-(pyrimidin-2-yl)pyridine-2-carboxylate was reacted with ammonia to give the title compound (55.6 mg, 60%) as a white solid. LC-MS (ES, m/z): 414 [M+H]⁺. ¹H NMR (300 MHz, CD₃OD) δ 9.07-9.00 (m, 4H), 8.39 (d, J=1.2 Hz, 1H), 8.30 (d, J=7.2 Hz 1H), 7.59-7.53 (m, 3H), 3.51-3.48 (m, 2H), 2.96 (s, 3H), 2.69-2.61 (m, 1H), 2.38-2.33 (m, 1H).

### Example X5

### Synthesis of 3-amino-5-fluoro-6-[3-[(3R)-3-hydroxy-3-(5-methyl-1,2,4-oxadiazol-3-yl)but-1-yn-1-yl]phenyl]pyridine-2-carboxamide

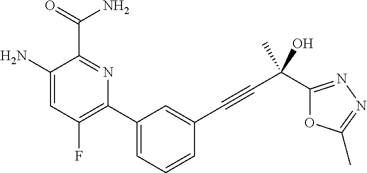 [SMILES: Cc1nnc([C@](C)(O)C#Cc2cccc(-c3nc(C(N)=O)c(N)cc3F)c2)o1]

### Step 1: Synthesis of methyl 3-amino-5-fluoro-6-[3-[(3R)-3-hydroxy-3-(5-methyl-1,3,4-oxadiazol-2-yl)but-1-yn-1-yl]phenyl]pyridine-2-carboxylate

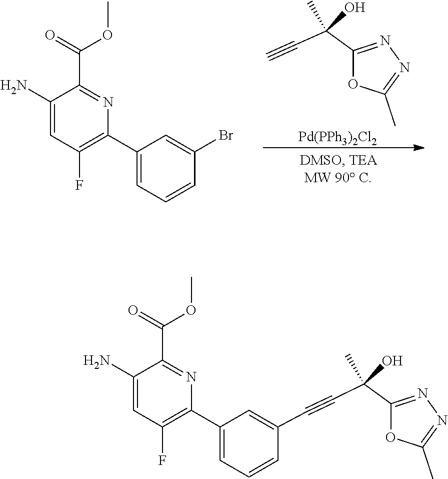 [SMILES: COC(=O)c1nc(-c2cccc(Br)c2)c(F)cc1N ; C#C[C@@](C)(O)c1nnc(C)o1 ; COC(=O)c1nc(-c2cccc(C#C[C@@](C)(O)c3nnc(C)o3)c2)c(F)cc1N ; "Pd(PPh3)2Cl2"]

Similar to as described in General Procedure G, methyl 3-amino-6-(3-bromophenyl)-5-fluoropyridine-2-carboxylate was reacted with (2R)-2-(5-methyl-1,3,4-oxadiazol-2-yl)but-3-yn-2-ol to give the title compound (250 mg, 68%) as a light yellow solid. LC-MS (ES, m/z): 397 [M+H]⁺.

### Step 2: Synthesis of 3-amino-5-fluoro-6-[3-[(3R)-3-hydroxy-3-(5-methyl-1,2,4-oxadiazol-3-yl)but-1-yn-1-yl]phenyl]pyridine-2-carboxamide

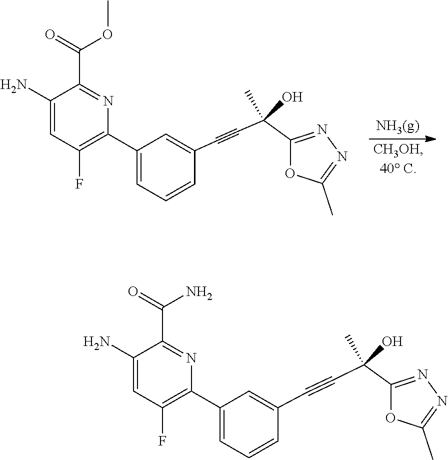 [SMILES: COC(=O)c1nc(-c2cccc(C#C[C@@](C)(O)c3nnc(C)o3)c2)c(F)cc1N ; Cc1nnc([C@](C)(O)C#Cc2cccc(-c3nc(C(N)=O)c(N)cc3F)c2)o1]

Similar to as described in General Procedure S, methyl 3-amino-5-fluoro-6-[3-[(3R)-3-hydroxy-3-(5-methyl-1,3,4-oxadiazol-2-yl)but-1-yn-1-yl]phenyl]pyridine-2-carboxylate was reacted with ammonia to give the title compound (98.1 mg, 57%) as a white solid. LC-MS (ES, m/z): 382 [M+H]⁺. ¹H NMR (300 MHz, CD₃OD) δ 7.99 (s, 1H), 7.93-7.90 (m, 1H), 7.48-7.41 (m, 1H), 6.98 (d, J=13.2 Hz, 1H), 2.57 (s, 3H), 1.98 (s, 3H).

### Example Y5

### Synthesis of 3-amino-5-fluoro-6-(3-[2-[(3R)-3-hydroxy-1-methyl-2-oxopiperidin-3-yl]ethynyl]phenyl)pyridine-2-carboxamide

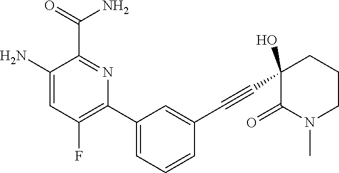 [SMILES: CN1CCC[C@@](O)(C#Cc2cccc(-c3nc(C(N)=O)c(N)cc3F)c2)C1=O]

### Step 1: Synthesis of methyl 3-amino-5-fluoro-6-(3-[2-[(3R)-3-hydroxy-1-methyl-2-oxopiperidin-3-yl]ethynyl]phenyl)pyridine-2-carboxylate

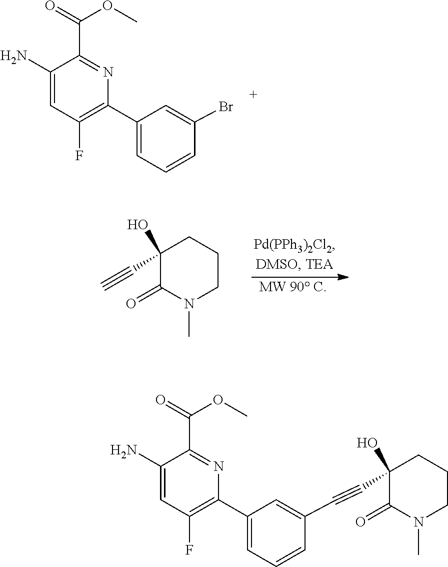 [SMILES: COC(=O)c1nc(-c2cccc(Br)c2)c(F)cc1N ; C#C[C@]1(O)CCCN(C)C1=O ; COC(=O)c1nc(-c2cccc(C#C[C@]3(O)CCCN(C)C3=O)c2)c(F)cc1N]

Similar to as described in General Procedure G, methyl 3-amino-6-(3-bromophenyl)-5-fluoropyridine-2-carboxylate was reacted with (3R)-3-ethynyl-3-hydroxy-1-methylpiperidin-2-one to give the title compound (100 mg, 82%) as a light yellow solid. LC-MS (ES, m/z): 398 [M+H]⁺.

### Step 2: Synthesis of 3-amino-5-fluoro-6-(3-[2-[(3R)-3-hydroxy-1-methyl-2-oxopiperidin-3-yl]ethynyl]phenyl)pyridine-2-carboxamide

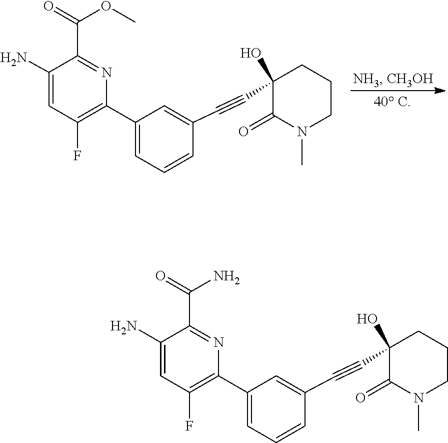 [SMILES: COC(=O)c1nc(-c2cccc(C#C[C@]3(O)CCCN(C)C3=O)c2)c(F)cc1N ; CN1CCC[C@@](O)(C#Cc2cccc(-c3nc(C(N)=O)c(N)cc3F)c2)C1=O]

Similar to as described in General Procedure S, methyl 3-amino-5-fluoro-6-(3-[2-[(3R)-3-hydroxy-1-methyl-2-oxopiperidin-3-yl]ethynyl]phenyl)pyridine-2-carboxylate was reacted with ammonia to give the title compound (40 mg, 21%) as a white solid. LC-MS (ES, m/z): 383 [M+H]⁺. ¹H NMR (300 MHz, CD₃OD) δ 7.96 (s, 1H), 7.92-7.89 (m, 2H), 7.45-7.40 (m, 2H), 7.01 (d, J=13.2 Hz, 1H), 3.55-3.32 (m, 2H), 2.99 (s, 3H), 2.37-2.07 (m, 4H).

### Example Z5

### Synthesis of 3-amino-5-fluoro-6-[3-[(3R)-3-hydroxy-3-(5-methyl-1,2,4-oxadiazol-3-yl)but-1-yn-1-yl]phenyl]pyridine-2-carboxamide

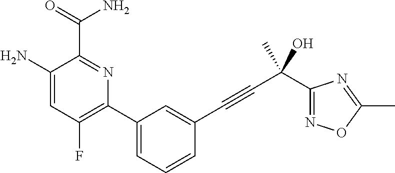 [SMILES: Cc1nc([C@](C)(O)C#Cc2cccc(-c3nc(C(N)=O)c(N)cc3F)c2)no1]

### Step 1: Synthesis of methyl 3-amino-5-fluoro-6-[3-[(3R)-3-hydroxy-3-(5-methyl-1,2,4-oxadiazol-3-yl)but-1-yn-1-yl]phenyl]pyridine-2-carboxylate

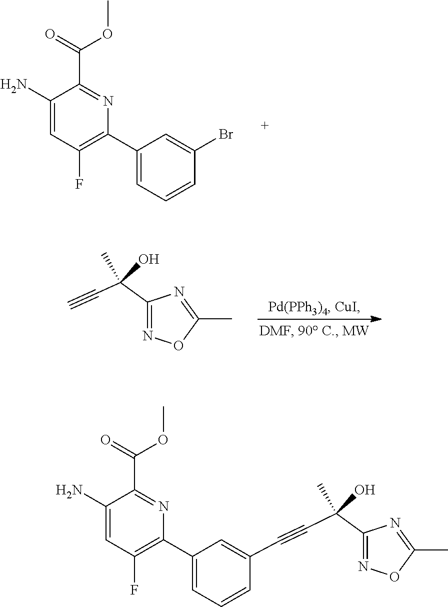

Similar to as described in General Procedure E, methyl 3-amino-6-(3-bromophenyl)-5-fluoropyridine-2-carboxylate (100 mg, 0.31 mmol, 1.00 equiv) was reacted with (R)-3-ethynyl-3-hydroxy-1-methylpyrrolidin-2-one to give the title compound (50 mg, 41%) as a yellow solid. LC-MS (ES, m/z): 397 [M+H]⁺.

### Step 2: Synthesis of 3-amino-5-fluoro-6-[3-[(3R)-3-hydroxy-3-(5-methyl-1,2,4-oxadiazol-3-yl)but-1-yn-1-yl]phenyl]pyridine-2-carboxamide

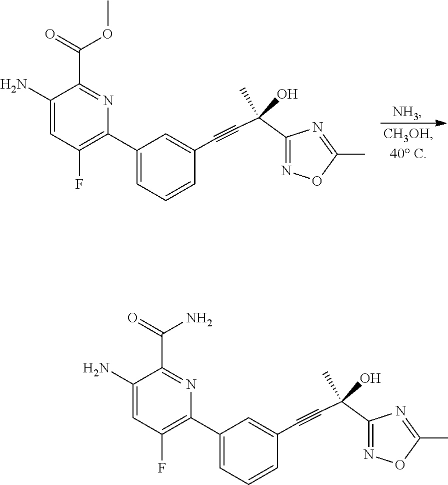 [SMILES: COC(=O)c1nc(-c2cccc(C#C[C@@](C)(O)c3noc(C)n3)c2)c(F)cc1N ; "NH3," ; Cc1nc([C@](C)(O)C#Cc2cccc(-c3nc(C(N)=O)c(N)cc3F)c2)no1]

Similar to as described in General Procedure S, methyl 3-amino-5-fluoro-6-[3-[(3R)-3-hydroxy-3-(5-methyl-1,2,4-oxadiazol-3-yl)but-1-yn-1-yl]phenyl]pyridine-2-carboxylate was reacted with ammonia to give the title compound (23.2 mg, 19%) as a white solid. LC-MS (ES, m/z): 382 [M+H]⁺. ¹H NMR (300 MHz, CD₃OD) δ 8.17 (s, 1H), 7.93-7.91 (m, 1H), 7.54-7.43 (m, 2H), 7.03 (d, J=13.2 Hz, 1H), 2.68 (s, 3H), 2.20 (s, 3H).

### Example A6

### Synthesis of 5-(3-[2-[(3R)-3-hydroxy-1-methyl-2-oxopyrrolidin-3-yl]ethynyl]phenyl)-2-methylbenzamide

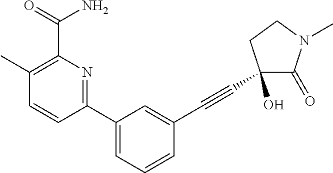

### Step 1: Synthesis of 2-(methoxycarbonyl)-3-methylpyridin-1-ium-1-olate

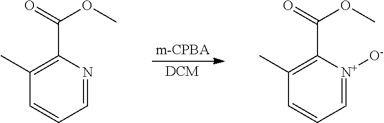

A solution of methyl 3-methylpyridine-2-carboxylate (800 mg, 5.29 mmol, 1.00 equiv) and 3-chloroperoxybenzoic acid (1826 mg, 10.58 mmol, 2.00 equiv) in dichloromethane (20 mL) was stirred for 4 hours at 45° C. The resulting solution was concentrated under vacuum and the residue was purified by a silica gel column eluting with ethyl acetate/petroleum ether (1:3). This resulted in the title compound (850 mg, 96%) as yellow oil. LC-MS (ES, m/z): 168 [M+H]⁺.

### Step 2: Synthesis of methyl 6-chloro-3-methylpyridine-2-carboxylate

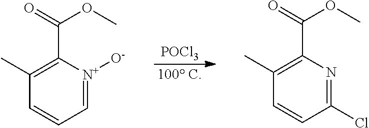

A solution of 2-(methoxycarbonyl)-3-methylpyridin-1-ium-1-olate (500 mg, 2.99 mmol, 1.00 equiv) in phosphorus oxychloride (2 mL) was stirred for 4 hours at 110° C. The reaction was quenched with water, adjusted pH to 7, and extracted with ethyl acetate. The organic layers were combined, dried over anhydrous sodium sulfate, and concentrated under vacuum. The residue was purified by a silica gel column eluting with ethyl acetate/petroleum ether (1:5) to give the title compound (230 mg, 41%) as a white solid. LC-MS (ES, m/z): 186 [M+H]⁺.

### Step 3: Synthesis of methyl 6-(3-[2-[(3R)-3-hydroxy-1-methyl-2-oxopyrrolidin-3-yl]ethynyl]phenyl)-3-methyl pyridine-2-carboxylate

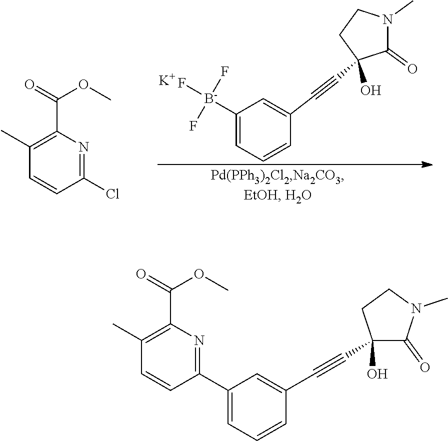

Similar to as described in General Procedure U, methyl 6-chloro-3-methylpyridine-2-carboxylate was reacted with potassium (R)-trifluoro(3-((3-hydroxy-1-methyl-2-oxopyrrolidin-3-yl)ethynyl)phenyl)borate to give the title compound (80 mg, 24%) as a red solid. LC-MS (ES, m/z): 365 [M+H]⁺.

### Step 4: Synthesis of 5-(3-[2-[(3R)-3-hydroxy-1-methyl-2-oxopyrrolidin-3-yl]ethynyl]phenyl)-2-methylbenzamide

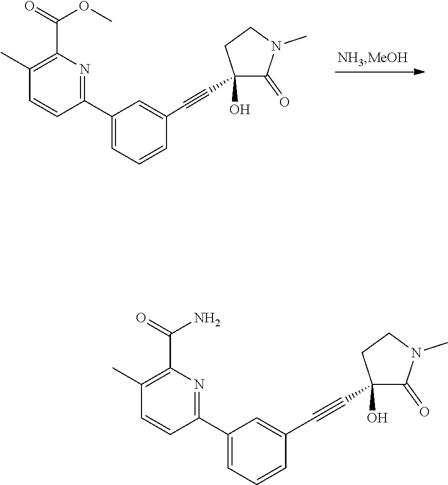

Similar to as described in General Procedure S, methyl 5-(3-[2-[(3R)-3-hydroxy-1-methyl-2-oxopyrrolidin-3-yl]ethynyl]phenyl)-2-methylbenzoate was reacted with ammonia to give the title compound (55 mg, 72%) as a white solid. LC-MS (ES, m/z): 350 [M+H]⁺. ¹H NMR (300 MHz, CD₃OD) δ 8.24 (d, J=1.5 Hz, 1H), 8.14 (d, J=7.2 Hz, 1H), 7.97 (d, J=8.1 Hz, 1H), 7.83 (d, J=8.7 Hz, 1H), 7.53-7.50 (m, 2H), 3.51-3.48 (m, 1H), 2.96 (s, 3H), 2.68 (s, 3H), 2.67-2.66 (m, 1H), 2.37-2.28 (m, 1H).

### Example B6

### Synthesis of 6-(2-fluoro-5-[2-[(3R)-3-hydroxy-1-methyl-2-oxopyrrolidin-3-yl]ethynyl]phenyl)-3-[(2-methoxyethyl)amino]pyrazine-2-carboxamide

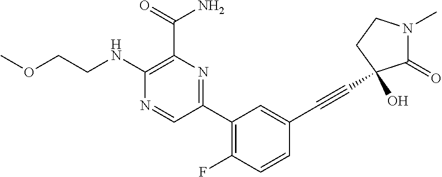

### Step 1: Synthesis of 3-chloro-N-(2-methoxyethyl)pyrazin-2-amine

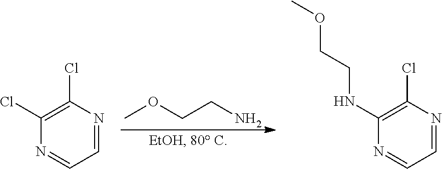

Similar to as described in General Procedure A, 2,3-dichloropyrazine was reacted with 2-methoxyethan-1-amine to give the title compound (4 g, 79%) as a white solid. LC-MS (ES, m/z): 189 [M+H]⁺.

### Step 2: Synthesis of methyl 3-[(2-methoxyethyl)amino]pyrazine-2-carboxylate

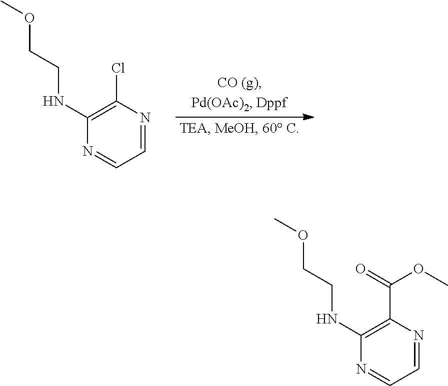

Similar to as described in General Procedure O, 3-chloro-N-(2-methoxyethyl)pyrazin-2-amine was reacted with carbon monoxide to give the title compound (761 mg, 34%) as yellow oil. LC-MS (ES, m/z): 212 [M+H]⁺.

### Step 3: Synthesis of methyl 6-bromo-3-[(2-methoxyethyl)amino]pyrazine-2-carboxylate

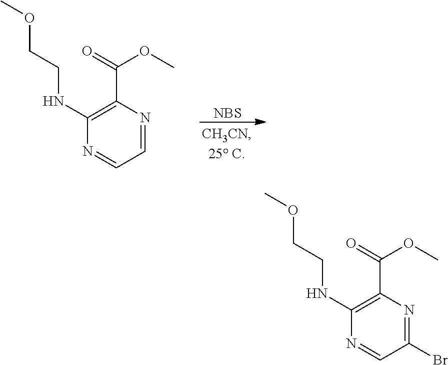

A solution of methyl 3-[(2-methoxyethyl)amino]pyrazine-2-carboxylate (700.00 mg, 3.31 mmol, 1.00 equiv) and NBS (710 mg, 3.99 mmol, 1.20 equiv) in acetonitrile (50 mL) was stirred for 4 h at 25° C. The resulting solution was concentrated under vacuum and the residue was purified by a silica gel column chromatography eluting with ethyl acetate/petroleum ether (1:10). This resulted in the title compound (690 mg, 72%) as a yellow solid. LC-MS (ES, m/z): 290 [M+H]⁺.

### Step 4: Synthesis of methyl 6-(5-bromo-2-fluorophenyl)-3-[(2-methoxyethyl)amino]pyrazine-2-carboxylate

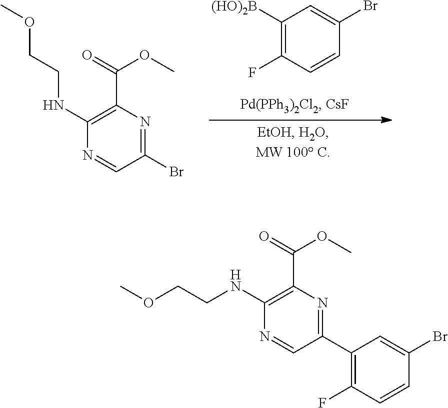 [SMILES: COCCNc1ncc(Br)nc1C(=O)OC ; OB(O)c1cc(Br)ccc1F ; COCCNc1ncc(-c2cc(Br)ccc2F)nc1C(=O)OC]

Similar to as described in General Procedure X, methyl 6-bromo-3-[(2-methoxyethyl)amino]pyrazine-2-carboxylate was reacted with 5-bromo-2-fluorophenylboronic acid to give the title compound (315 mg, 79%) as light yellow oil. LC-MS (ES, m/z): 385 [M+H]⁺.

### Step 5: Synthesis of methyl 6-(2-fluoro-5-[2-[(3R)-3-hydroxy-1-methyl-2-oxopyrrolidin-3-yl]ethynyl]phenyl)-3-[(2-methoxyethyl)amino]pyrazine-2-carboxylate

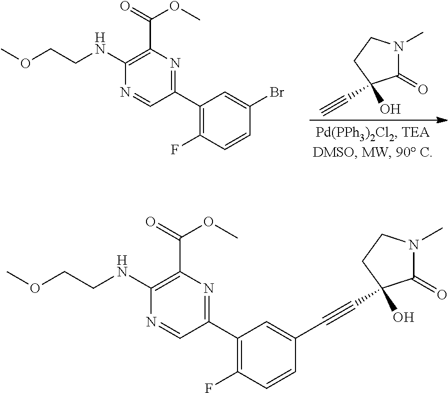

Similar to as described in General Procedure G, methyl 6-(5-bromo-2-fluorophenyl)-3-[(2-methoxyethyl)amino]pyrazine-2-carboxylate was reacted with (R)-3-ethynyl-3-hydroxy-1-methylpyrrolidin-2-one to give the title compound (205 mg, 89%) as a light yellow solid. LC-MS (ES, m/z): 443 [M+H]⁺.

### Step 6: Synthesis of 6-(2-fluoro-5-[2-[(3R)-3-hydroxy-1-methyl-2-oxopyrrolidin-3-yl]ethynyl]phenyl)-3-[(2-methoxyethyl)amino]pyrazine-2-carboxamide

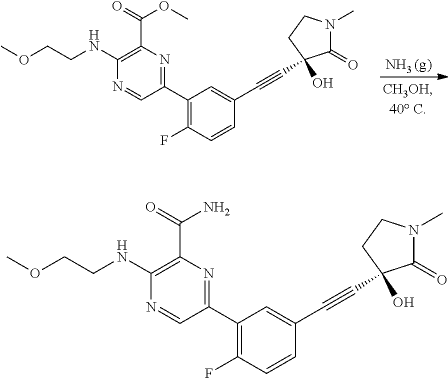

Similar to as described in General Procedure S, 6-(2-fluoro-5-[2-[(3R)-3-hydroxy-1-methyl-2-oxopyrrolidin-3-yl]ethynyl]phenyl)-3-[(2-methoxyethyl)amino]pyrazine-2-carboxylate was reacted with ammonia to give the title compound (39.1 mg, 20%) as a light yellow solid. LC-MS (ES, m/z): 428 [M+H]⁺. ¹H NMR (300 MHz, DMSO-d₆) δ 9.01-8.01 (m, 1H), 8.66 (d, J=2.7 Hz, 1H), 8.38 (s, 1H), 8.18-8.14 (m, 1H), 7.76 (s, 1H), 7.48-7.42 (m, 1H), 7.38-7.31 (m, 1H), 6.44 (s, 1H), 3.67-3.61 (m, 2H), 3.54-3.51 (m, 2H), 3.37-3.32 (m, 2H), 3.30 (s, 1H), 2.79 (s, 3H), 2.51-2.45 (m, 1H), 2.43-2.13 (m, 1H).

### Example C6

### Synthesis of 6-(3-[2-[(3R)-3-hydroxy-1-methyl 2-oxopyrrolidin-3-yl]ethynyl]phenyl)-4-(trifluoromethyl)pyridine-2-carboxamide

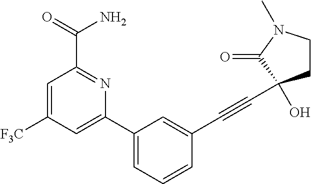 [SMILES: CN1CC[C@@](O)(C#Cc2cccc(-c3cc(C(F)(F)F)cc(C(N)=O)n3)c2)C1=O]

### Step 1: Synthesis of methyl 4-(trifluoromethyl)pyridine-2-carboxylate

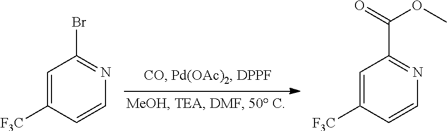

Similar to as described in General Procedure O, 2-bromo-4-(trifluoromethyl)pyridine was reacted with carbon monoxide to give the title compound (1.9 g, 70%) as a red oil. LC-MS (ES, m/z): 206 [M+H]⁺.

### Step 2: Synthesis of 2-(methoxycarbonyl)-4-(trifluoromethyl)pyridin-1-ium-1-olate

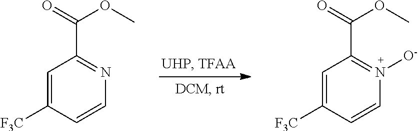

A solution of methyl 4-(trifluoromethyl)pyridine-2-carboxylate (500 mg, 2.44 mmol, 1.00 equiv), urea peroxide (459 mg, 4.63 mmol, 2.00 equiv), and trifluoroacetic anhydride (1024 mg, 4.88 mmol, 2.00 equiv) in dichloromethane (10 mL) was stirred for 14 h at room temperature. The solids were filtered out and the filtrate was concentrated under vacuum to give the title compound (400 mg, 74%) as a yellow solid. LC-MS (ES, m/z): 222 [M+H]⁺.

### Step 3: Synthesis of methyl 6-chloro-4-(trifluoromethyl)pyridine-2-carboxylate

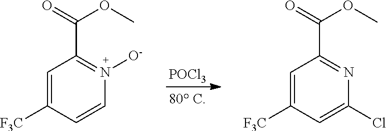

A solution of 2-(methoxycarbonyl)-4-(trifluoromethyl)pyridin-1-ium-1-olate (400 mg, 1.81 mmol, 1.00 equiv) in phosphorus oxychloride (10 mL) was stirred for 14 hours at 60° C. The resulting solution was concentrated under vacuum and the residue was purified by a silica gel column chromatography eluting with ethyl acetate/petroleum ether (1:5). This resulted in the title compound (300 mg, 69%) as a yellow solid. LC-MS (ES, m/z): 240 [M+H]⁺.

### Step 4: Synthesis of (R)-ethyl 6-(3-((3-hydroxy-1-methyl-2-oxopyrrolidin-3-yl)ethynyl)phenyl)-4-(trifluoromethyl)picolinate

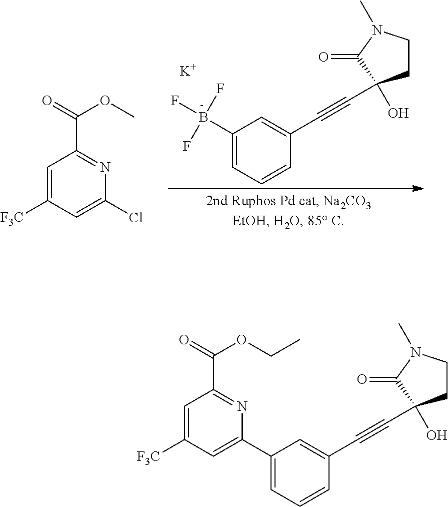

Similar to as described in General Procedure U, methyl 6-chloro-4-(trifluoromethyl)pyridine-2-carboxylate was reacted with potassium (R)-trifluoro(3-((3-hydroxy-1-methyl-2-oxopyrrolidin-3-yl)ethynyl)phenyl)borate to give the title compound (230 mg, 61%) as a yellow solid. LC-MS (ES, m/z): 433 [M+H]⁺.

### Step 5: Synthesis of 6-(3-[2-[(3R)-3-hydroxy-1-methyl 2-oxopyrrolidin-3-yl]ethynyl]phenyl)-4-(trifluoromethyl)pyridine-2-carboxamide

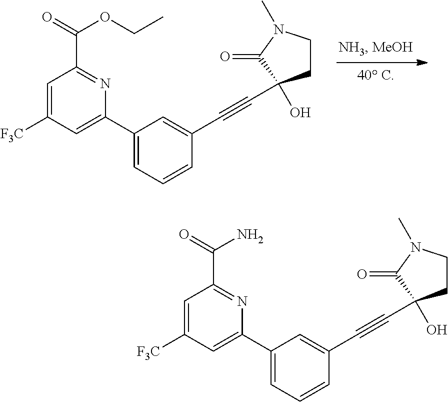 [SMILES: CCOC(=O)c1cc(C(F)(F)F)cc(-c2cccc(C#C[C@]3(O)CCN(C)C3=O)c2)n1 ; CN1CC[C@@](O)(C#Cc2cccc(-c3cc(C(F)(F)F)cc(C(N)=O)n3)c2)C1=O]

Similar to as described in General Procedure S, ethyl 6-(3-[2-[(3R)-3-hydroxy-1-methyl-2-oxopyrrolidin-3-yl]ethynyl]phenyl)-4-(trifluoromethyl)pyridine-2-carboxylate was reacted with ammonia to give the title compound (70 mg, 33%) as a white solid. LC-MS (ES, m/z): 404 [M+H]⁺. ¹H NMR (300 Mhz, CDCl₃) δ 8.39 (t, J=1.5 Hz, 2H), 8.30-8.25 (m, 2H), 7.63-7.52 (m, 2H), 3.53-3.45 (m, 2H), 2.94 (s, 3H), 2.66-2.58 (m, 1H), 2.38-2.28 (m, 1H).

### Example D6

### Synthesis of 4-(3-[2-[(3R)-3-hydroxy-1-methyl-2-oxopyrrolidin-3-yl]ethynyl]phenyl)-6-methoxypyrimidine-2-carboxamide

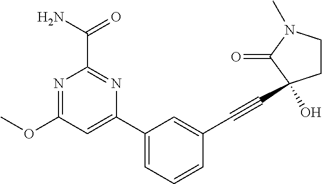 [SMILES: COc1cc(-c2cccc(C#C[C@]3(O)CCN(C)C3=O)c2)nc(C(N)=O)n1]

### Step 1: Synthesis of (3R)-3-[2-[3-(2-chloro-6-methoxypyrimidin-4-yl)phenyl]ethynyl]-3-hydroxy-1-methyl pyrrolidin-2-one

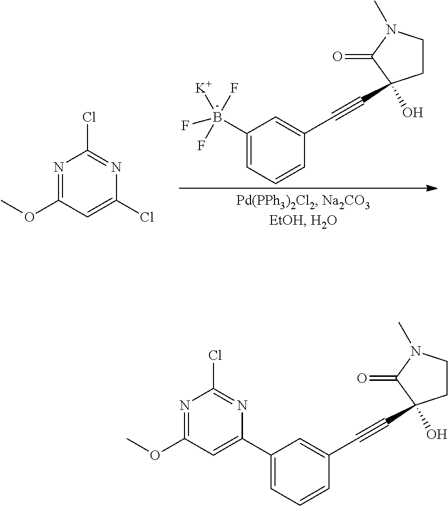

Similar to as described in General Procedure U, 2,4-dichloro-6-methoxypyrimidine was reacted with potassium (R)-trifluoro(3-((3-hydroxy-1-methyl-2-oxopyrrolidin-3-yl)ethynyl)phenyl)borate to give the title compound (80 mg, 8.5%) as an off-white solid. LC-MS (ES, m/z): 358 [M+H]⁺.

### Step 2: Synthesis of ethyl 4-(3-[2-[(3R)-3-hydroxy-1-methyl-2-oxopyrrolidin-3-yl]ethynyl]phenyl)-6-methoxypyrimidine-2-carboxylate

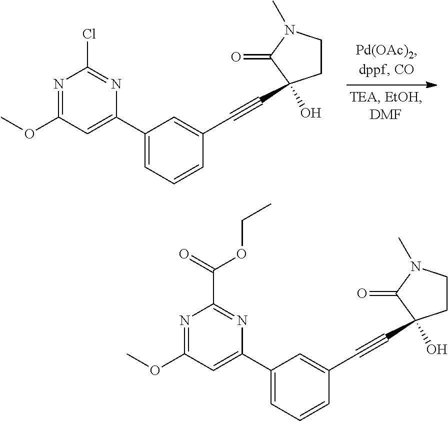 [SMILES: COc1cc(-c2cccc(C#C[C@]3(O)CCN(C)C3=O)c2)nc(Cl)n1 ; CCOC(=O)c1nc(OC)cc(-c2cccc(C#C[C@]3(O)CCN(C)C3=O)c2)n1]

Similar to as described in General Procedure O, (3R)-3-[2-[3-(2-chloro-6-methoxypyrimidin-4-yl)phenyl]ethynyl]-3-hydroxy-1-methylpyrrolidin-2-one was reacted with carbon monoxide to give the title compound (36 mg, 65%) as a light brown solid. LC-MS (ES, m/z): 396 [M+H]⁺.

### Step 3: Synthesis of 4-(3-[2-[(3R)-3-hydroxy-1-methyl-2-oxopyrrolidin-3-yl]ethynyl]phenyl)-6-methoxypyrimidine-2-carboxamide

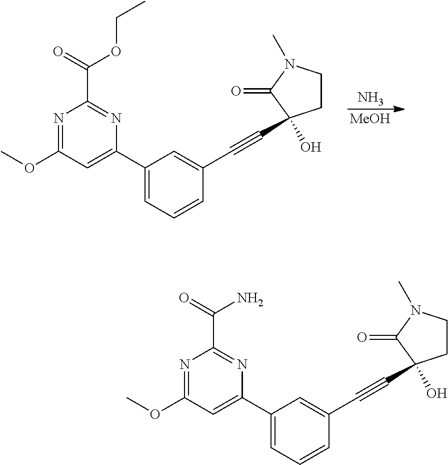

Similar to as described in General Procedure S, ethyl 4-(3-[2-[(3R)-3-hydroxy-1-methyl-2-oxopyrrolidin-3-yl]ethynyl]phenyl)-6-methoxypyrimidine-2-carboxylate was reacted with ammonia to give the title compound (6.3 mg, 19%) as a white solid. LC-MS (ES, m/z): 367 [M+H]⁺. ¹H NMR (400 MHz, CD₃OD) δ 8.28 (s, 1H), 8.15 (d, J=7.6 Hz, 1H), 7.52 (d, J=8.0 Hz, 1H), 7.49-7.37 (m, 2H), 4.03 (s, 3H), 3.43-3.34 (m, 2H), 2.83 (s, 3H), 2.53-2.47 (m, 1H), 2.25-2.18 (m, 1H).

### Synthesis of potassium (R)-trifluoro(3-(3-hydroxy-3-(5-methyl-1,3,4-oxadiazol-2-yl)but-1-yn-1-yl)phenyl)borate

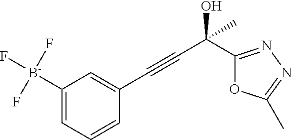 [SMILES: Cc1nnc([C@](C)(O)C#Cc2cccc([B-](F)(F)F)c2)o1]

Potassium trifluoro-(3-iodophenyl)boranuide (1 eq) is brought up in a solution of 1:1 triethylamine (14 eq) and N,N-dimethylformamide (26 eq). The solution was purged with nitrogen before addition of cuprous iodide (0.05 eq), bis(triphenylphosphine)palladium(II) dichloride (0.05 eq) and (R)-2-(5-methyl-1,3,4-oxadiazol-2-yl)but-3-yn-2-ol (1.05 eq) at once. The reaction mixture was stirred at 40° C. overnight (18 hrs) whereupon the reaction mixture was concentrated under vacuum to yield a brown oil. Water was added and the solution was sonicated until an orange-brown solid crashed out of solution. The solid was filtered off and the aqueous layer was concentrated under high vacuum to afford a yellow oil in quantitative yield that is used without further purification.

### Synthesis of ethyl 2-chloro-6-(1-methyl-1H-pyrazol-5-yl)pyrimidine-4-carboxylate

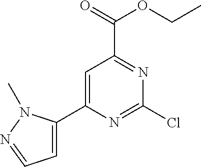 [SMILES: CCOC(=O)c1cc(-c2ccnn2C)nc(Cl)n1]

To a solution of 2-chloro-6-(2-methyl pyrazol-3-yl)pyrimidine-4-carboxylic acid (1.15 g) in ethanol (0.25 M) was added hydrochloric acid (4 mol/L in 1,4-dioxane, 4 eq). The reaction mixture was refluxed for 1.5 hours at 120° C. then slowly cooled to ambient temperature and stirred overnight. The reaction mixture was subsequently concentrated to dryness and triturated with methanol to afford ethyl 2-chloro-6-(1-methyl-1H-pyrazol-5-yl)pyrimidine-4-carboxylate (608 mg) as an off-white solid.

### Example E6

### Synthesis of 2-[3-[(3R)-3-hydroxy-3-(5-methyl-1,3,4-oxadiazol-2-yl)but-1-ynyl]phenyl]-6-(2-methylpyrazol-3-yl)pyrimidine-4-carboxamide

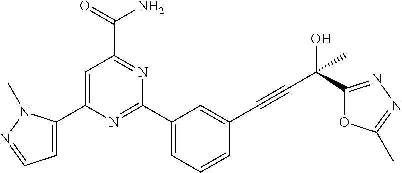 [SMILES: Cc1nnc([C@](C)(O)C#Cc2cccc(-c3nc(C(N)=O)cc(-c4ccnn4C)n3)c2)o1]

Similar to as described in General Procedure U, ethyl 2-chloro-6-(1-methyl-1H-pyrazol-5-yl)pyrimidine-4-carboxylate (78 mg) was reacted with potassium (R)-trifluoro(3-(3-hydroxy-3-(5-methyl-1,3,4-oxadiazol-2-yl)but-1-yn-1-yl)phenyl)borate to give (R)-ethyl 2-(3-(3-hydroxy-3-(5-methyl-1,3,4-oxadiazol-2-yl)but-1-yn-1-yl)phenyl)-6-(1-methyl-1H-pyrazol-5-yl)pyrimidine-4-carboxylate which was taken onto the next step without purification. Similar to as described in General Procedure I, (R)-ethyl 2-(3-(3-hydroxy-3-(5-methyl-1,3,4-oxadiazol-2-yl)but-1-yn-1-yl)phenyl)-6-(1-methyl-1H-pyrazol-5-yl)pyrimidine-4-carboxylate was reacted to afford 27.9 mg of the title compound (22.2%). M+H=430.2; 1H NMR (400 MHz, DMSO-d6) δ 8.76-8.71 (m, 2H), 8.68 (dt, J=7.7, 1.5 Hz, 1H), 8.24 (s, 1H), 8.04 (s, 1H), 7.68 (dt, J=7.7, 1.5 Hz, 1H), 7.66-7.58 (m, 2H), 7.31 (d, J=2.1 Hz, 1H), 7.04 (s, 1H), 4.38 (s, 3H), 2.56 (s, 3H), 1.95 (s, 3H).

### Example F6

### Synthesis of 2-[3-[(3R)-3-hydroxy-3-(5-methyl-1,3,4-oxadiazol-2-yl)but-1-ynyl]phenyl]pyrimidine-4-carboxamide

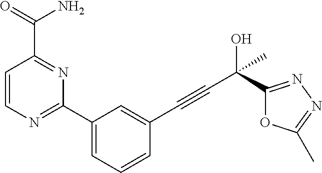 [SMILES: Cc1nnc([C@](C)(O)C#Cc2cccc(-c3nccc(C(N)=O)n3)c2)o1]

Similar to as described in General Procedure U, ethyl 2-chloropyrimidine-4-carboxylate (80 mg) was reacted with potassium (R)-trifluoro(3-(3-hydroxy-3-(5-methyl-1,3,4-oxadiazol-2-yl)but-1-yn-1-yl)phenyl)borate to give (R)-ethyl 2-(3-(3-hydroxy-3-(5-methyl-1,3,4-oxadiazol-2-yl)but-1-yn-1-yl)phenyl)pyrimidine-4-carboxylate which was taken onto the next step without purification. Similar to as described in General Procedure I, (R)-ethyl 2-(3-(3-hydroxy-3-(5-methyl-1,3,4-oxadiazol-2-yl)but-1-yn-1-yl)phenyl)pyrimidine-4-carboxylate was reacted to afford 7.4 mg of the title compound (4.8%).

M+H=350.2; 1H NMR (400 MHz, DMSO-d6) δ 9.14 (d, J=5.0 Hz, 1H), 8.71-8.65 (m, 3H), 7.99 (s, 1H), 7.94 (d, J=4.9 Hz, 1H), 7.67-7.56 (m, 2H), 7.03 (s, 1H), 2.55 (s, 3H), 1.94 (s, 3H).

### Example G6

### Synthesis of 2-[3-[2-[(3R)-3-hydroxy-1-methyl-2-oxo-pyrrolidin-3-yl]ethynyl]phenyl]-6-pyrazol-1-yl-pyrimidine-4-carboxamide

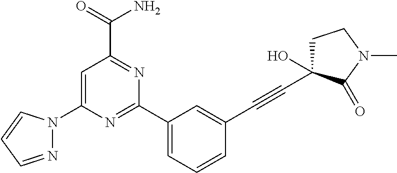 [SMILES: CN1CC[C@@](O)(C#Cc2cccc(-c3nc(C(N)=O)cc(-n4cccn4)n3)c2)C1=O]

Similar to as described in General Procedure A, methyl 2,6-dichloropyrimidine-4-carboxylate (500 mg) in 1-butanol (0.25 M) was reacted with sodium bicarbonate (2 eq) and pyrazole (1 eq) to afford methyl 2-chloro-6-pyrazol-1-ylpyrimidine-4-carboxylate (60 mg) after purification. Similar to as described in General Procedure U, methyl 2-chloro-6-pyrazol-1-ylpyrimidine-4-carboxylate (60 mg) was reacted with potassium (S)-trifluoro(3-((3-hydroxy-1-methyl-2-oxopyrrolidin-3yl)ethynyl)phenyl)borate to form ethyl 2-[3-[2-[(3R)-3-hydroxy-1-methyl-2-oxo-pyrrolidin-3-yl]ethynyl]phenyl]-6-pyrazol-1-ylpyrimidine-4-carboxylate which was taken onto the next step without purification. Similar to as described in General Procedure I, ethyl 2-[3-[2-[(3R)-3-hydroxy-1-methyl-2-oxo-pyrrolidin-3-yl]ethynyl]phenyl]-6-pyrazol-1-ylpyrimidine-4-carboxylate was reacted to form 23.4 mg of the title compound (21.4%). M+H=403.2; ¹H NMR (400 MHz, DMSO-d6) δ 9.04 (d, J=2.8 Hz, 1H), 8.79 (dt, J=7.7, 1.6 Hz, 1H), 8.74 (q, J=2.0 Hz, 2H), 8.29 (s, 1H), 8.08 (s, 1H), 8.04 (d, J=1.6 Hz, 1H), 7.67-7.57 (m, 2H), 6.75 (dd, J=2.8, 1.6 Hz, 1H), 6.50 (s, 1H), 3.38 (t, J=6.4 Hz, 4H), 2.82 (s, 3H), 2.56-2.51 (m, 1H), 2.28-2.16 (m, 1H).

### Example H6

### Synthesis of 6-[3-[2-[(3R)-3-hydroxy-1-methyl-2-oxo-pyrrolidin-3-yl]ethynyl]phenyl]-5-methyl-pyridine-2-carboxamide

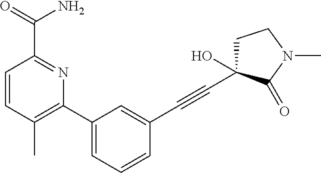 [SMILES: Cc1ccc(C(N)=O)nc1-c1cccc(C#C[C@]2(O)CCN(C)C2=O)c1]

Similar to as described in General Procedure B, 6-chloro-5-methyl-pyridine-2-carboxylic acid (250 mg) was reacted with ammonium chloride to give 6-chloro-5-methyl-pyridine-2-carboxamide which was taken into the next step without purification. Similar to as described in General Procedure U, 6-chloro-5-methyl-pyridine-2-carboxamide was reacted with potassium (S)-trifluoro(3-((3-hydroxy-1-methyl-2-oxopyrrolidin-3yl)ethynyl)phenyl)borate to form 75.9 mg of the title compound (47.9%). M+H=350.2; ¹H NMR (400 MHz, DMSO-d6) δ 7.98 (s, 1H), 7.96-7.87 (m, 2H), 7.69-7.64 (m, 2H), 7.61-7.55 (m, 1H), 7.56-7.47 (m, 2H), 6.46 (s, 1H), 3.35 (t, J=6.3 Hz, 2H), 2.79 (s, 3H), 2.48-2.40 (m, 1H), 2.37 (s, 3H), 2.22-2.13 (m, 1H).

### Example I6

### Synthesis of

2-[3-[2-[(3R)-3-hydroxy-1-methyl-2-oxo-pyrrolidin-3-yl]ethynyl]phenyl]-6-(2-methylimidazol-1-yl)pyrimidine-4-carboxamide

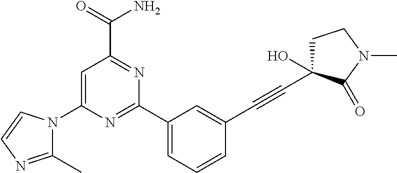 [SMILES: Cc1nccn1-c1cc(C(N)=O)nc(-c2cccc(C#C[C@]3(O)CCN(C)C3=O)c2)n1]

Similar to as described in General Procedure A, methyl 2,6-dichloropyrimidine-4-carboxylate (500 mg) was added to a pre-stirred (for 30 minutes) solution of sodium hydride (1.05 eq) and methyl imidazole (1 eq) to afford methyl 2-chloro-6-(2-methylimidazol-1-yl)pyrimidine-4-carboxylate (160 mg) after purification and NMR structure confirmation. Similar to as described in General Procedure U, methyl 2-chloro-6-(2-methylimidazol-1-yl)pyrimidine-4-carboxylate (145 mg) was reacted with potassium (S)-trifluoro(3-((3-hydroxy-1-methyl-2-oxopyrrolidin-3yl)ethynyl)phenyl)borate to form ethyl 2-[3-[2-[(3R)-3-hydroxy-1-methyl-2-oxo-pyrrolidin-3-yl]ethynyl]phenyl]-6-(2-methylimidazol-1-yl)pyrimidine-4-carboxylate which was taken onto the next step without purification. Similar to as described in General Procedure J, ethyl 2-[3-[2-[(3R)-3-hydroxy-1-methyl-2-oxo-pyrrolidin-3-yl]ethynyl]phenyl]-6-(2-methylimidazol-1-yl)pyrimidine-4-carboxylate was reacted to form 2-[3-[2-[(3R)-3-hydroxy-1-methyl-2-oxo-pyrrolidin-3-yl]ethynyl]phenyl]-6-(2-methylimidazol-1-yl)pyrimidine-4-carboxylic acid (240 mg). This intermediate was taken onto the next step without purification. Similar to as described in General Procedure B, 2-[3-[2-[(3R)-3-hydroxy-1-methyl-2-oxo-pyrrolidin-3-yl]ethynyl]phenyl]-6-(2-methylimidazol-1-yl)pyrimidine-4-carboxylic acid was reacted with ammonium chloride to give 10.1 mg of the title compound (4.1%).

M+H=417.2; ¹H NMR (400 MHz, DMSO-d6) δ 8.75 (s, 1H), 8.69-8.60 (m, 2H), 8.12-8.02 (m, 3H), 7.68-7.56 (m, 2H), 7.02 (d, J=1.7 Hz, 1H), 6.48 (s, 1H), 3.38 (dd, J=7.4, 5.6 Hz, 2H), 2.82 (s, 3H), 2.79 (s, 3H), 2.49-2.44 (m, 1H), 2.27-2.15 (m, 1H).

### Example J6 and Example K6

### Synthesis of 2-(3-(((R)-3-hydroxy-1-methyl-2-oxopyrrolidin-3-yl)ethynyl)phenyl)-5-((S)-tetrahydrofuran-3-ylamino)pyrimidine-4-carboxamide and 2-(3-(((R)-3-hydroxy-1-methyl-2-oxopyrrolidin-3-yl)ethynyl)phenyl)-5-((R)-tetrahydrofuran-3-ylamino)pyrimidine-4-carboxamide

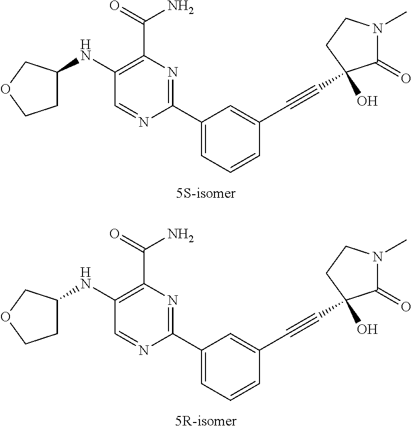 [SMILES: CN1CC[C@@](O)(C#Cc2cccc(-c3ncc(N[C@H]4CCOC4)c(C(N)=O)n3)c2)C1=O ; CN1CC[C@@](O)(C#Cc2cccc(-c3ncc(N[C@@H]4CCOC4)c(C(N)=O)n3)c2)C1=O]

### Step 1: Synthesis of ethyl 2-chloro-5-(tetrahydrofuran-3-ylamino)pyrimidine-4-carboxylate

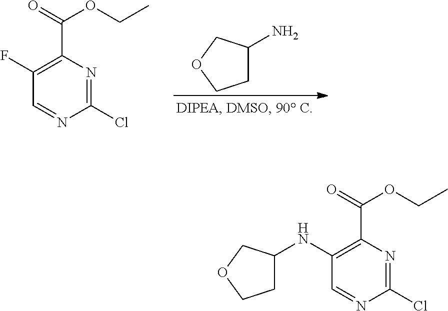 [SMILES: CCOC(=O)c1nc(Cl)ncc1F ; NC1CCOC1 ; CCOC(=O)c1nc(Cl)ncc1NC1CCOC1]

Similar to as described in General Procedure A, ethyl 2-chloro-5-fluoropyrimidine-4-carboxylate was reacted with oxolan-3-amine to give the title compound (535 mg, 77%) as a yellow solid. LC-MS (ES, m/z): 272 [M+H]⁺.

### Step 2: Synthesis of 2-chloro-5-(tetrahydrofuran-3-ylamino)pyrimidine-4-carboxamide

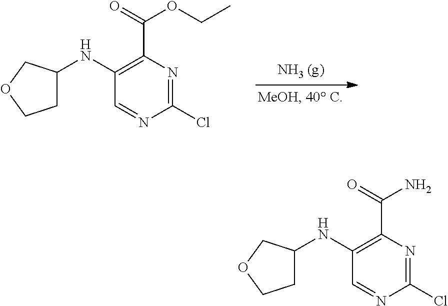 [SMILES: CCOC(=O)c1nc(Cl)ncc1NC1CCOC1 ; NC(=O)c1nc(Cl)ncc1NC1CCOC1]

Similar to as described in General Procedure S, ethyl 2-chloro-5-[(oxolan-3-yl)amino]pyrimidine-4-carboxylate was reacted with ammonia to give the title compound (340 mg, 76%) as a yellow solid. LC-MS (ES, m/z): 243 [M+H]⁺.

### Step 3: Synthesis of 2-(3-(((R)-3-hydroxy-1-methyl-2-oxopyrrolidin-3-yl)ethynyl)phenyl)-5-((S)-tetrahydrofuran-3-ylamino)pyrimidine-4-carboxamide and 2-(3-(((R)-3-hydroxy-1-methyl-2-oxopyrrolidin-3-yl)ethynyl)phenyl)-5-((R)-tetrahydrofuran-3-ylamino)pyrimidine-4-carboxamide

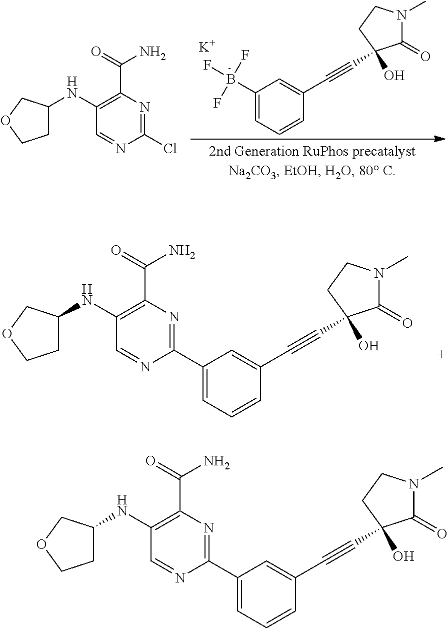

Similar to as described in General Procedure U, 2-chloro-5-[(oxolan-3-yl)amino]pyrimidine-4-carboxamide was reacted with potassium (R)-trifluoro(3-((3-hydroxy-1-methyl-2-oxopyrrolidin-3-yl)ethynyl)phenyl)borate to give the title compounds as a R/S mixture. After chiral separation 69.6 mg (12%) of the 5R-isomer (yellow solid) and 86.4 mg (15%) of the 5S-isomer (yellow solid) were isolated. The 5R-isomer: t_{R}=2.28 min (CHIRALPAK IA-3, 0.46*5 cm, MeOH=100%, 1.0 ml/min); The 5S-isomer: t_{R}=5.09 min (CHIRALPAK IA-3, 0.46*5 cm, MeOH=100%, 1.0 ml/min). Both isomers showed identical LC-MS and ¹H NMR as shown below. LC-MS (ES, m/z): 422 [M+H]⁺. ¹H NMR (300 Mhz, DMSO-d₆) δ 8.69 (s, 1H), 8.61 (s, 1H), 8.48-8.37 (m, 3H), 7.87 (s, 1H), 7.50-7.42 (m, 2H), 6.47 (s, 1H), 4.37 (br, 1H), 3.96-3.76 (m, 3H), 3.63-3.60 (m, 1H), 3.38-3.31 (m, 2H), 2.81 (s, 3H), 2.49-2.14 (m, 3H), 1.81-1.77 (m, 1H).

### Example L6

### Synthesis of (R)-6-(3-((3-hydroxy-1-methyl-2-oxopyrrolidin-3-yl)ethynyl)phenyl)-4-(1-methyl-1H-pyrazol-5-yl)picolinamide

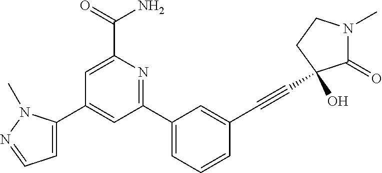 [SMILES: CN1CC[C@@](O)(C#Cc2cccc(-c3cc(-c4ccnn4C)cc(C(N)=O)n3)c2)C1=O]

### Step 1: Synthesis of (R)-ethyl 6-(3-((3-hydroxy-1-methyl-2-oxopyrrolidin-3-yl)ethynyl)phenyl)-4-(1-methyl-1H-pyrazol-5-yl)picolinate

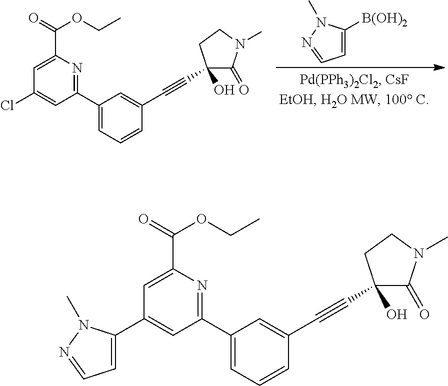

Similar to as described in General Procedure X, ethyl 4-chloro-6-(3-[2-[(3R)-3-hydroxy-1-methyl-2-oxopyrrolidin-3-yl]ethynyl]phenyl)pyridine-2-carboxylate was reacted with (1-methyl-1H-pyrazol-5-yl) boronic acid to give the title compound (115 mg, 52%) as a brown solid. LC-MS (ES, m/z): 445 [M+H]⁺.

### Step 2: Synthesis of (R)-6-(3-((3-hydroxy-1-methyl-2-oxopyrrolidin-3-yl)ethynyl)phenyl)-4-(1-methyl-1H-pyrazol-5-yl)picolinamide

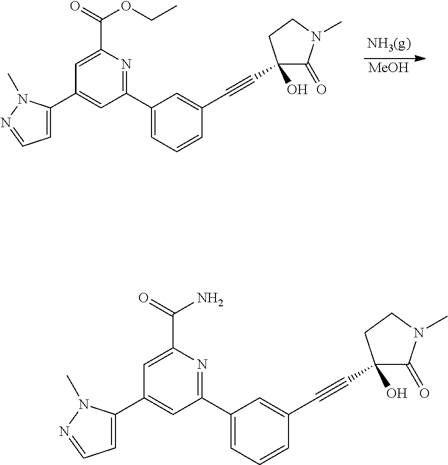 [SMILES: "MeOH" ; CCOC(=O)c1cc(-c2ccnn2C)cc(-c2cccc(C#C[C@]3(O)CCN(C)C3=O)c2)n1 ; CN1CC[C@@](O)(C#Cc2cccc(-c3cc(-c4ccnn4C)cc(C(N)=O)n3)c2)C1=O]

Similar to as described in General Procedure S, ethyl 6-(3-[2-[(3R)-3-hydroxy-1-methyl-2-oxopyrrolidin-3-yl]ethynyl]phenyl)-4-(1-methyl-1H-pyrazol-5-yl)pyridine-2-carboxylate was reacted with ammonia to give the title compound (26.3 mg, 28%) as a white solid. LC-MS (ES, m/z): 416 [M+H]⁺. ¹HNMR (300 MHz, CD₃OD) δ 8.36 (s, 1H), 8.26-8.19 (m, 3H), 7.59-7.49 (m, 3H), 6.70 (s, 1H), 4.03 (s, 3H), 3.51-3.46 (m, 2H), 2.92 (s, 3H), 2.64-2.56 (m, 1H), 2.36-2.27 (m, 1H).

### Example M6

### Synthesis of (R)-5-amino-2-(3-((3-hydroxy-1-methyl-2-oxopyrrolidin-3-yl)ethynyl)phenyl)pyrimidine-4-carboxamide

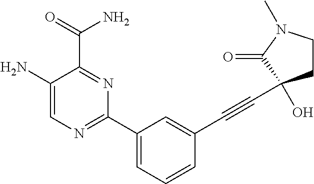 [SMILES: CN1CC[C@@](O)(C#Cc2cccc(-c3ncc(N)c(C(N)=O)n3)c2)C1=O]

### Step 1: Synthesis of (R)-ethyl 5-amino-2-(3-((3-hydroxy-1-methyl-2-oxopyrrolidin-3-yl)ethynyl)phenyl)pyrimidine-4-carboxylate

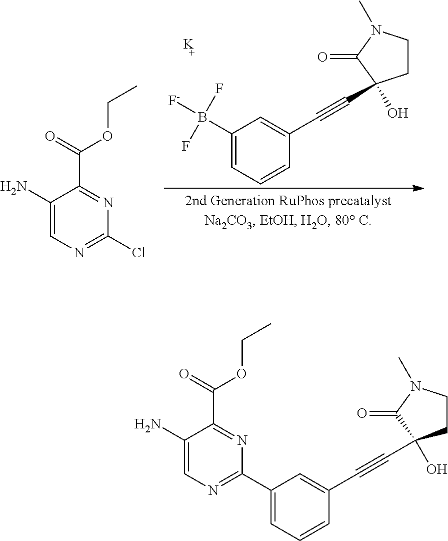

Similar to as described in General Procedure U, ethyl 5-amino-2-chloropyrimidine-4-carboxylate was reacted with potassium (R)-trifluoro(3-((3-hydroxy-1-methyl-2-oxopyrrolidin-3-yl)ethynyl)phenyl)borate to give the title compound (40 mg, 18%) as a yellow solid. LC-MS (ES, m/z): 381 [M+H]⁺.

### Step 2: Synthesis of (R)-5-amino-2-(3-((3-hydroxy-1-methyl-2-oxopyrrolidin-3-yl)ethynyl)phenyl)pyrimidine-4-carboxamide

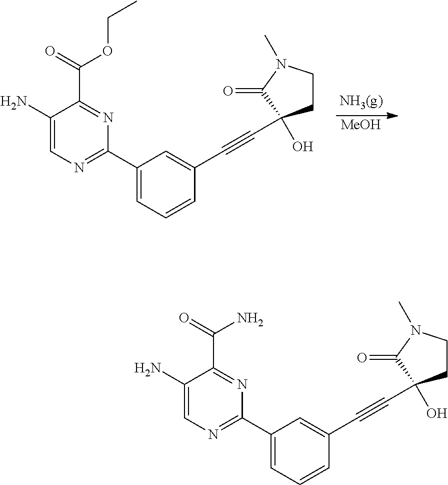 [SMILES: "MeOH" ; CCOC(=O)c1nc(-c2cccc(C#C[C@]3(O)CCN(C)C3=O)c2)ncc1N ; CN1CC[C@@](O)(C#Cc2cccc(-c3ncc(N)c(C(N)=O)n3)c2)C1=O]

Similar to as described in General Procedure S, ethyl 5-amino-2-(3-[2-[(3R)-3-hydroxy-1-methyl-2-oxopyrrolidin-3-yl]ethynyl]phenyl)pyrimidine-4-carboxylate was reacted with ammonia to give the title compound (15.5 mg, 42%) as a light yellow solid. LC-MS (ES, m/z): 352 [M+H]⁺. ¹HNMR (300 MHz, DMSO-d₆) δ 8.53 (s, 1H), 8.45-8.39 (m, 3H), 7.73 (s, 1H), 7.35 (m, 2H), 7.04 (s, 2H), 6.46 (s, 1H), 3.38-3.35 (m, 2H), 2.81 (s, 3H), 2.46-2.42 (m, 1H), 2.23-2.14 (m, 1H).

### Example N6

### Synthesis of (R)-2-(3-((3-hydroxy-1-methyl-2-oxopyrrolidin-3-yl)ethynyl)phenyl)-5-(2,2,2-trifluoroethyl amino)pyrimidine-4-carboxamide

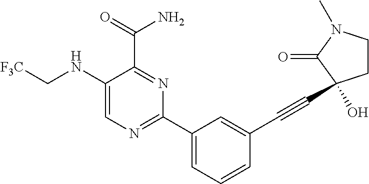 [SMILES: CN1CC[C@@](O)(C#Cc2cccc(-c3ncc(NCC(F)(F)F)c(C(N)=O)n3)c2)C1=O]

### Step 1: Synthesis of ethyl 2-chloro-5-(2,2,2-trifluoroethylamino)pyrimidine-4-carboxylate

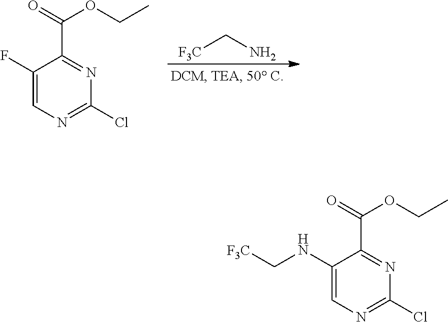 [SMILES: CCOC(=O)c1nc(Cl)ncc1F ; NCC(F)(F)F ; CCOC(=O)c1nc(Cl)ncc1NCC(F)(F)F]

Similar to as described in General Procedure A, ethyl 2-chloro-5-fluoropyrimidine-4-carboxylate was reacted with 2,2,2-trifluoroethan-1-amine to give the title compound (180 mg, 13%) as a yellow solid. LC-MS (ES, m/z): 284 [M+H]⁺.

### Step 2: Synthesis of 2-chloro-5-(2,2,2-trifluoroethylamino)pyrimidine-4-carboxamide

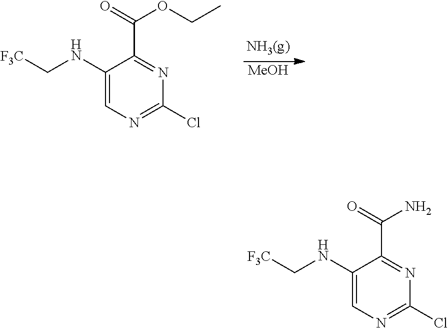 [SMILES: "MeOH" ; CCOC(=O)c1nc(Cl)ncc1NCC(F)(F)F ; NC(=O)c1nc(Cl)ncc1NCC(F)(F)F]

Similar to as described in General Procedure S, ethyl 2-chloro-5-[(2,2,2-trifluoroethyl)amino]pyrimidine-4-carboxylate was reacted with ammonia to give the title compound (80 mg, 50%) as a light yellow solid. LC-MS (ES, m/z): 255 [M+H]⁺.

### Step 3: Synthesis of (R)-2-(3-((3-hydroxy-1-methyl-2-oxopyrrolidin-3-yl)ethynyl)phenyl)-5-(2,2,2-trifluoroethylamino)pyrimidine-4-carboxamide

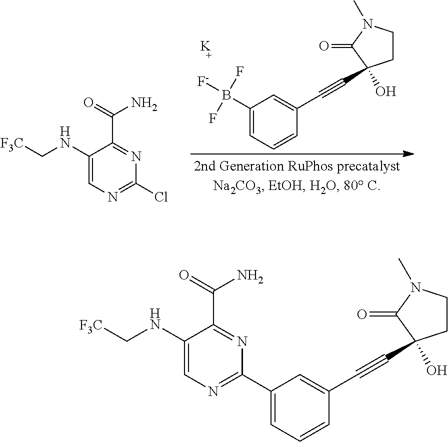

Similar to as described in General Procedure U, 2-chloro-5-(2,2,2-trifluoroethylamino)pyrimidine-4-carboxamide was reacted with potassium (R)-trifluoro(3-((3-hydroxy-1-methyl-2-oxopyrrolidin-3-yl)ethynyl)phenyl)borate to give the title compound 4.4 mg (3%) as an off-white solid. LC-MS (ES, m/z): 434 [M+H]⁺. ¹HNMR (400 MHz, CDCl₃) δ 8.59 (s, 2H), 8.35 (s, 1H), 8.25 (d, J=7.6 Hz, 1H), 8.01 (s, 1H), 7.51-7.48 (m, 1H), 7.38 (m, 1H), 5.77 (s, 1H), 3.96-3.92 (m, 2H), 3.58-3.49 (m, 1H), 3.44-3.36 (m, 1H), 2.98 (s, 3H), 2.70-2.65 (m, 1H), 2.42-2.35 (m, 1H).

### Example O6

### Synthesis of

4-(3-[2-[(3R)-3-hydroxy-1-methyl-2-oxopyrrolidin-3-yl]ethynyl]phenyl)-5-[(2,2,2-trifluoroethyl)amino]pyrimidine-2-carboxamide

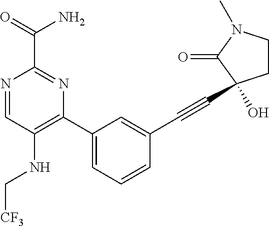 [SMILES: CN1CC[C@@](O)(C#Cc2cccc(-c3nc(C(N)=O)ncc3NCC(F)(F)F)c2)C1=O]

The title compound was isolated from Example N6 as a side product (1.4 mg, 1%); off-white solid; LC-MS (ES, m/z): 434 [M+H]⁺. ¹HNMR: (300 MHz, CD₃OD) δ 8.55 (s, 1H), 8.50 (d, J=1.8 Hz, 1H), 8.39-8.36 (m, 1H), 7.51-7.42 (m, 2H), 4.15 (m, 2H), 3.55-3.45 (m, 2H), 2.95 (s, 3H), 2.65-2.57 (m, 1H), 2.38-2.28 (m, 1H).

### Example P6 and Example Q6

### Synthesis of 6-(2-fluoro-5-(((R)-3-hydroxy-1-methyl-2-oxopyrrolidin-3-yl)ethynyl)phenyl)-3-((R)-1-methoxypropan-2-ylamino)picolinamide and 6-(2-fluoro-5-(((R)-3-hydroxy-1-methyl-2-oxopyrrolidin-3-yl)ethynyl)phenyl)-3-((S)-1-methoxypropan-2-ylamino)picolinamide

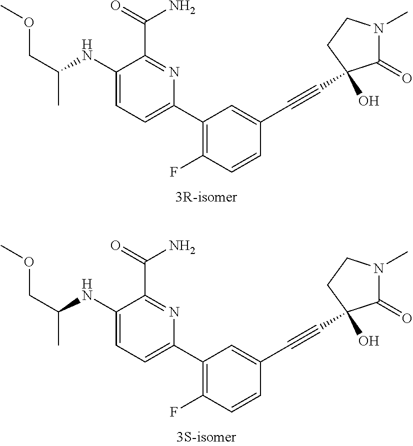

### Step 1: Synthesis of ethyl 3-[(1-methoxypropan-2-yl)amino]pyridine-2-carboxylate

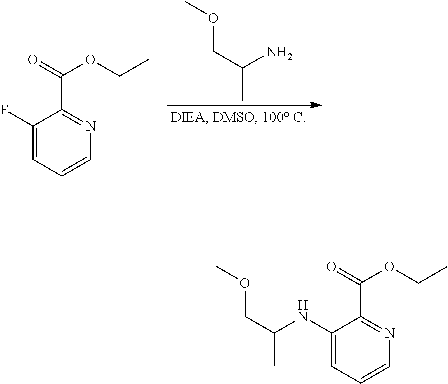

Similar to as described in General Procedure A, ethyl 3-fluoropyridine-2-carboxylate was reacted with 1-methoxypropan-2-amine to give the title compound (1.2 g, 57%) as a light red solid. LC-MS (ES, m/z): 239 [M+H]⁺.

### Step 2: Synthesis of ethyl 6-bromo-3-[(1-methoxypropan-2-yl)amino]pyridine-2-carboxylate

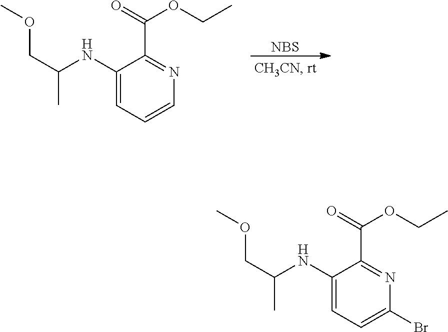

A solution of ethyl 3-[(1-methoxypropan-2-yl)amino]pyridine-2-carboxylate (1.20 g, 5.04 mmol, 1.00 equiv) and NBS (1.08 g, 6.07 mmol, 1.20 equiv) in acetonitrile (40 mL) was stirred for 4 h at 25° C. The resulting solution was concentrated under vacuum and the residue was purified by a silica gel column chromatography eluting with ethyl acetate/petroleum ether (1:10) to give the title compound (1.4 g, 88%) as light yellow oil. LC-MS (ES, m/z): 317, 319 [M+H]⁺.

### Step 3: Synthesis of ethyl 6-(5-bromo-2-fluorophenyl)-3-[(1-methoxypropan-2-yl)amino]pyridine-2-carboxylate

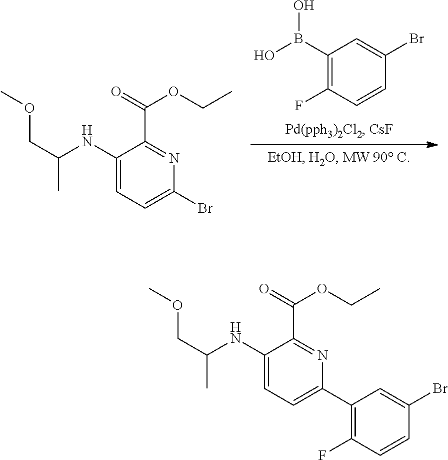 [SMILES: CCOC(=O)c1nc(Br)ccc1NC(C)COC ; OB(O)c1cc(Br)ccc1F ; CCOC(=O)c1nc(-c2cc(Br)ccc2F)ccc1NC(C)COC]

Similar to as described in General Procedure X, ethyl 6-bromo-3-[(1-methoxypropan-2-yl)amino]pyridine-2-carboxylate was reacted with 5-bromo-2-fluorophenylboronic acid to give the title compound (470 mg, 91%) as a light yellow oil. LC-MS (ES, m/z): 411, 413 [M+H]⁺.

### Step 4: Synthesis of ethyl 6-(2-fluoro-5-[2-[(3R)-3-hydroxy-1-methyl-2-oxopyrrolidin-3-yl]ethynyl]phenyl)-3-[(1-methoxypropan-2-yl)amino]pyridine-2-carboxylate

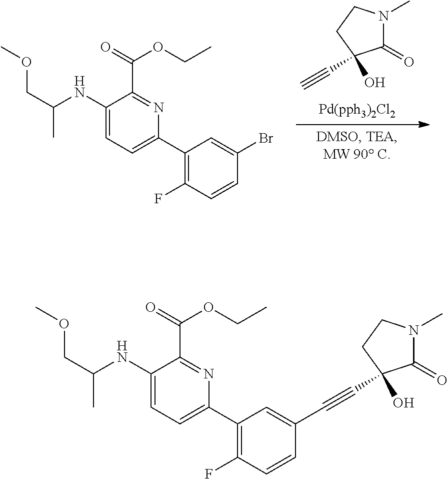

Similar to as described in General Procedure G, ethyl 6-(5-bromo-2-fluorophenyl)-3-[(1-methoxypropan-2-yl)amino]pyridine-2-carboxylate was reacted with (R)-3-ethynyl-3-hydroxy-1-methylpyrrolidin-2-one to give the title compound (215 mg, 94%) as a light yellow solid. LC-MS (ES, m/z): 470 [M+H]⁺.

### Step 5: Synthesis of 6-(2-fluoro-5-(((R)-3-hydroxy-1-methyl-2-oxopyrrolidin-3-yl)ethynyl)phenyl)-3-((R)-1-methoxypropan-2-ylamino)picolinamide and 6-(2-fluoro-5-(((R)-3-hydroxy-1-methyl-2-oxopyrrolidin-3-yl)ethynyl)phenyl)-3-((S)-1-methoxypropan-2-ylamino)picolinamide

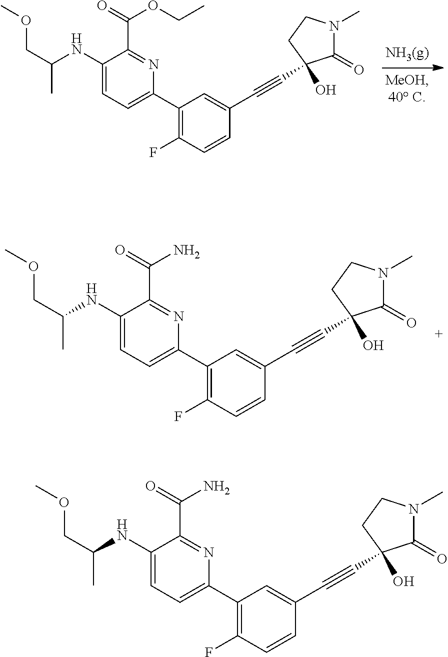

Similar to as described in General Procedure S, ethyl 6-(2-fluoro-5-[2-[(3R)-3-hydroxy-1-methyl-2-oxopyrrolidin-3-yl]ethynyl]phenyl)-3-[(1-methoxypropan-2-yl)amino]pyridine-2-carboxylate was reacted with ammonia to give the title compounds as a R/S mixture. After chiral separation 19.2 mg (10%) of the 3R-isomer (white solid) and 21.5 mg (11%) of the 3S-isomer (white solid) were isolated. The 3R-isomer: t_{R}=2.64 min (Lux Cellulose-4, 0.46*5 cm, Hex:EtOH=50:50, 1.0 ml/min); The 3S-isomer: t_{R}=3.59 min (Lux Cellulose-4, 0.46*5 cm, Hex:EtOH=50:50, 1.0 ml/min). Both isomers showed identical LC-MS and ¹H NMR as shown below. LC-MS (ES, m/z): 441 [M+H]⁺. ¹HNMR (400 MHz, CD₃OD) δ 8.11 (dd, J=2.0 Hz, 7.6 Hz, 1H), 7.81 (dd, J=2.0 Hz, 8.8 Hz, 1H), 7.46-7.43 (m, 1H), 7.36-7.33 (m, 1H), 7.21-7.16 (m, 1H), 3.88-3.85 (m, 1H), 3.52-3.47 (m, 4H), 3.41 (s, 3H), 2.94 (s, 3H), 2.63-2.57 (m, 1H), 2.35-2.32 (m, 1H).

Aryl Substitution Reactions

In the Scheme below, A₁-A₄ and R⁴-R⁶ are defined elsewhere in the application.

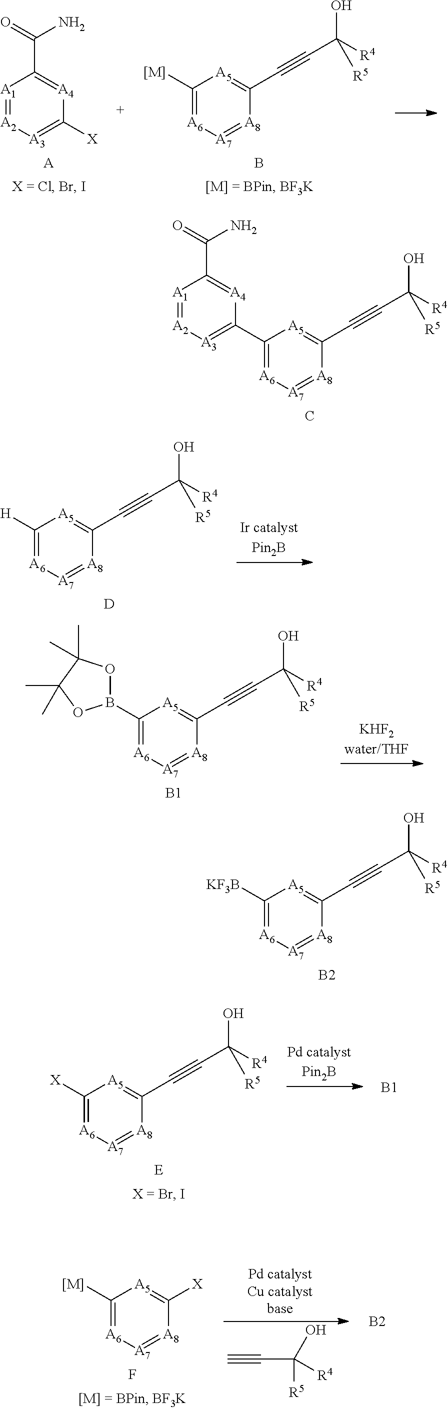

Compounds of type C can be prepared via Suzuki-type coupling of 3-alkynylaryl or heteroaryl boronic acids, esters (B1) or trifluoroborate salts (B2) to haloheterocycles A (Molander et al Acc. Chem. Res. 2007). Compounds of type B can be prepared by various routes including direct borylation of arenes containing sterically accessible C—H bonds (D) (Hartwig, J. F. et al. Chem. Rev. 2010) or Sonogashira coupling to aryl or heteroarylboronic esters or trifluoroborates (F).

### Preparation of Moieties Adjacent to the Alkynyl Portion

Moieties such as those shown below may be prepared as described in U.S. patent application Ser. No. 13/768,873, filed Feb. 15, 2013, and entitled “Tricyclic Compounds and Methods of Use Therefor,” which is incorporated herein by reference.

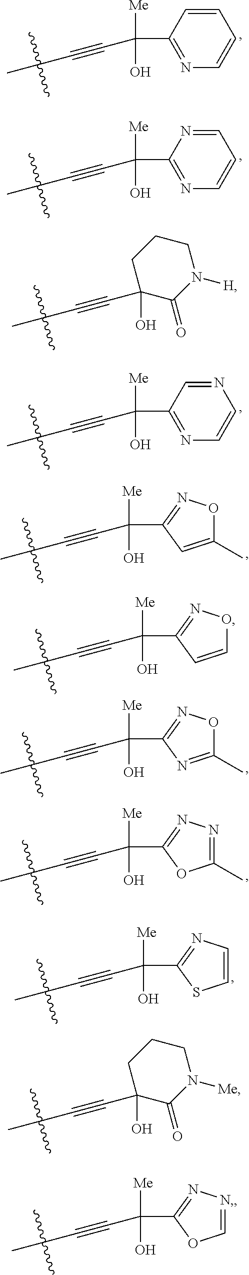

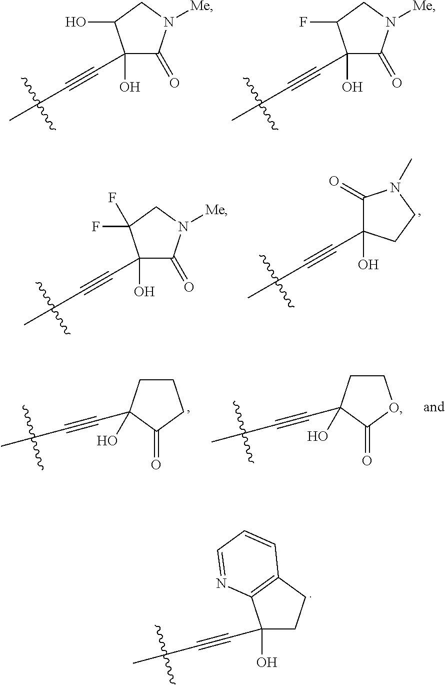

Optional methods for accessing these types of moieties are also described below.

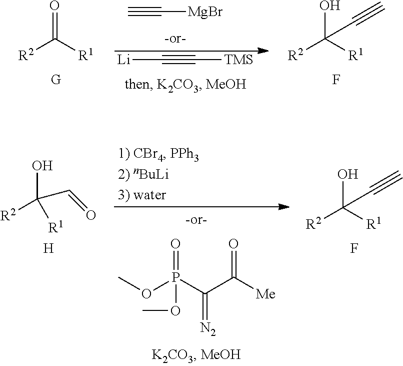

Terminal alkynes (F) for use in Sonogashira coupling reactions to haloarenes can be generated by a number of methods including those described in the above scheme. Addition of ethynylmagnesium bromide to substituted ketones (G) or addition of lithium trimethylsilylacetylide followed by proteolytic removal of the trimethylsilyl group will generate substituted propargyl alcohols. Alternatively, aldehydes (H) can be converted to terminal alkynes via a Corey-Fuchs process (steps 1-3 Corey Tetrahedron Lett. 1972) or via conditions for the Bestmann-Ohira modification of the Gilbert-Seyferth process (Bestmann Synthesis, 2004).

The following compounds were prepared using methodologies similar to those presented above:

| | | | LCMS |
|---|---|---|---|
| Ex. | Structure | Name | M + H |
| 150 | 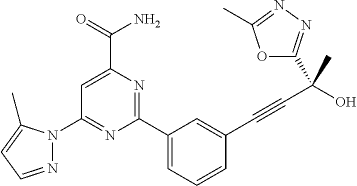 [SMILES: Cc1nnc([C@](C)(O)C#Cc2cccc(-c3nc(C(N)=O)cc(-n4nccc4C)n3)c2)o1] | 2-[3-](3R)-3-hydroxy-3-(5- methyl-1,3,4-oxadiazol-2-yl) but-1-ynyl]pheny1]-6-(5- methylpyrazol-1-yl)pyrimid ine-4-carboxamide | 430.1 |
| 151 | 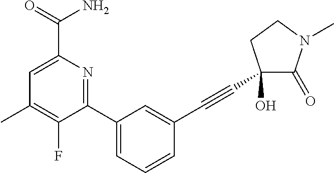 | 5-fluoro-6-[3-[2-[(3R)-3-hydroxy- l-methyl-2-oxo-pyrrolidin-3-yl] ethynyl]phenyl]-4-methyl- pyridine-2-carboxamide | 368.1 |
| 152 | 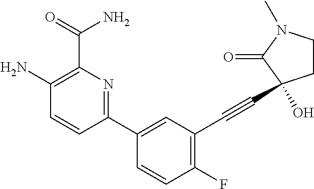 [SMILES: CN1CC[C@@](O)(C#Cc2cc(-c3ccc(N)c(C(N)=O)n3)ccc2F)C1=O] | 3-amino-6-[4-fluoro-3-[2-[(3R)- 3-hydroxy-l-methyl-2-oxo- pyrrolidin-3-yl]ethynyl]phenyl] pyridine-2-carboxamide | 369.05 |
| 153 | 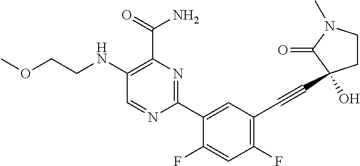 [SMILES: COCCNc1cnc(-c2cc(C#C[C@]3(O)CCN(C)C3=O)c(F)cc2F)nc1C(N)=O] | 2-[2,4-difluoro-5[2-[(3R)- 3-hydroxy-1-methyl-2-oxo- pyrrolidin-3-yl]ethynyl]phenyl]- 5-(2-methoxyethylamino) pyrimidine-4-carboxamide | 446.1 |
| 154 | 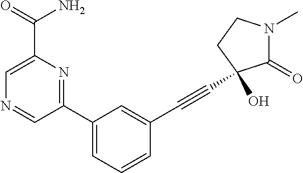 [SMILES: CN1CC[C@@](O)(C#Cc2cccc(-c3cncc(C(N)=O)n3)c2)C1=O] | 6-[3-[2-[(3R)-3-hydroxy-1- methyl-2-oxo-pyrrolidin-3- yl]ethynyl]phenyl]pyrazine- 2-carboxamide | 337.10 |
| 155 | 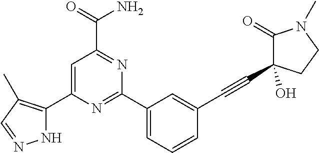 [SMILES: Cc1cn[nH]c1-c1cc(C(N)=O)nc(-c2cccc(C#C[C@]3(O)CCN(C)C3=O)c2)n1] | 2-[3-[2-[(3R)-3-hydroxy-l- methyl-2-oxo-pyrrolidin-3- yl]ethynyl]phenyl]-6-(4- methyl-1H-pyrazol-5-yl) pyrimidine-4-carboxamide | 417.15 |
| 156 | 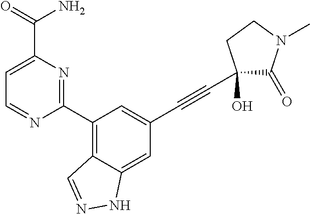 [SMILES: CN1CC[C@@](O)(C#Cc2cc(-c3nccc(C(N)=O)n3)c3cn[nH]c3c2)C1=O] | 2-[6-[2-[(3R)-3-hydroxy-1- methyl-2-oxo-pyrrolidin-3- yl]ethynyl]-1H-indazol-4-yl] pyrimidine-4-carboxamide | 375.0 |
| 157 | 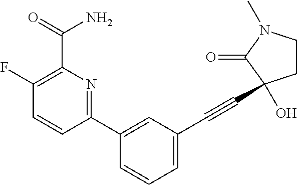 [SMILES: CN1CC[C@@](O)(C#Cc2cccc(-c3ccc(F)c(C(N)=O)n3)c2)C1=O] | 3-fluoro-6-[3-[2-[(3R)-3-hydroxy- l-methyl-2-oxo-pyrrolidin- 3-yl]ethynyl]phenyl] pyridine-2-carboxamide | 354.10 |
| 158 | 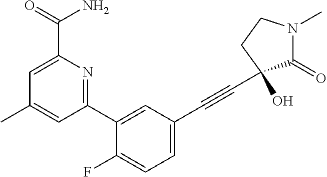 | 6-[2-fluoro-5-[2-[(3R)-3-hydroxy- 1-methyl-2-oxo-pyrrolidin- 3-yl]ethynyl]phenyl]-4-methyl- pyridine-2-carboxamide | 368.1 |
| 159 | 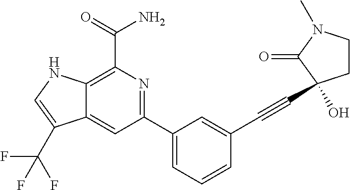 [SMILES: CN1CC[C@@](O)(C#Cc2cccc(-c3cc4c(C(F)(F)F)c[nH]c4c(C(N)=O)n3)c2)C1=O] | 5-[3-[2-[(3R)-3-hydroxy-l- methyl-2-oxo-pyrrolidin-3- yl]ethynyl]phenyl]-3- (trifluoromethyl)-1H-pyrrolo [2,3-c]pyridine-7-carboxamide | 443.15 |
| 160 | 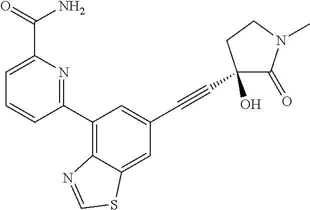 [SMILES: CN1CC[C@@](O)(C#Cc2cc(-c3cccc(C(N)=O)n3)c3ncsc3c2)C1=O] | 6-[6-[2-[(3R)-3-hydroxy-1- methyl-2-oxo-pyrrolidin-3- yl]ethynyl]-1,3-benzothiazol- 4-yl[pyridine-2-carboxamide | 393.1 |
| 161 | 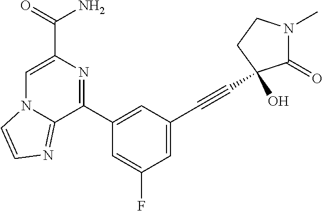 [SMILES: CN1CC[C@@](O)(C#Cc2cc(F)cc(-c3nc(C(N)=O)cn4ccnc34)c2)C1=O] | 8-[3-fluoro-5-[2-[(3R)-3-hydroxy- l-methyl-2-oxo-pyrrolidin-3- yl]ethynyl]phenyl]imidazol [1,2-a]pyrazine-6-carboxamide | 394.3 |
| 162 | 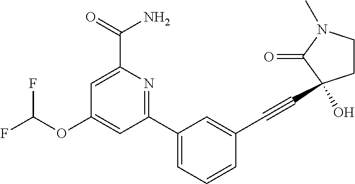 [SMILES: CN1CC[C@@](O)(C#Cc2cccc(-c3cc(OC(F)F)cc(C(N)=O)n3)c2)C1=O] | 4-(difluoromethoxy)-6-[3-[2- [(3R)-3-hydroxy-l-methyl- 2-oxo-pyrrolidin-3-yl]ethynyl] phenyl]pyridine-2-carboxamide | 402.2 |
| 163 | 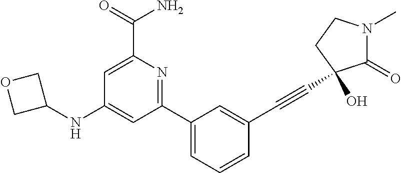 [SMILES: CN1CC[C@@](O)(C#Cc2cccc(-c3cc(NC4COC4)cc(C(N)=O)n3)c2)C1=O] | 6-[3-[2-[(3R)-3-hydroxy-1- methyl-2-oxo-pyrrolidin-3- yl]ethynyl]phenyl]-4-(oxetan- 3-ylamino)pyridine-2- carboxamide | 407.1 |
| 164 | 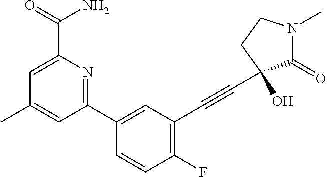 | 6-[4-fluoro-3-[2-[(3R)-3-hydroxy- 1-methyl-2-oxo-pyrrolidin-3-yl] ethynyl]phenyl]-4-methyl- pyridine-2-carboxamide | 368.10 |
| 165 | 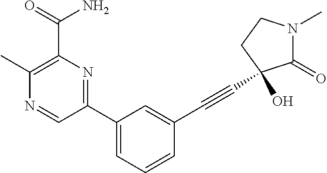 | 6-[3-[2-[(3R)-3-hydroxy-1- methyl-2-oxo-pyrrolidin-3- yl]ethynyl]phenyl]-3-methyl- pyrazine-2-carboxamide | 351.15 |
| 166 | 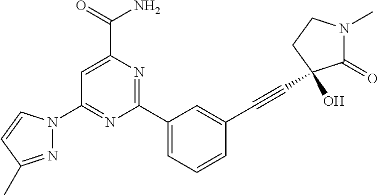 [SMILES: Cc1ccn(-c2cc(C(N)=O)nc(-c3cccc(C#C[C@]4(O)CCN(C)C4=O)c3)n2)n1] | 2-[3-[2-[(3R)-3-hydroxy-1- methyl-2-oxo-pyrrolidin-3- yl]ethynyl]phenyl]-6-(3- methylpyrazol-1-yl)pyrimidine- 4-carboxamide | 417.1 |
| 167 | 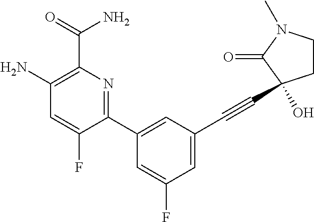 [SMILES: CN1CC[C@@](O)(C#Cc2cc(F)cc(-c3nc(C(N)=O)c(N)cc3F)c2)C1=O] | 3-amino-5-fluoro-6-[3-fluoro- 5-[2-[(3R)-3-hydroxy-1-methyl- 2-oxo-pyrrolidin-3-yl]ethynyl] phenyl]pyridine-2-carboxamide | 409.05 |
| 168 | 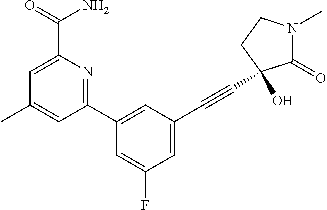 | 6-[3-fluoro-5-[2-[(3R)-3-hydroxy- 1-methyl-2-oxo-pyrrolidin-3-yl] ethynyl]phenyl]-4-methyl- pyridine-2-carboxamide | 368.1 |
| 169 | 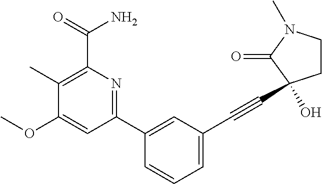 [SMILES: COc1cc(-c2cccc(C#C[C@]3(O)CCN(C)C3=O)c2)nc(C(N)=O)c1C] | 6-[3-[2-[(3R)-3-hydroxy-1- methyl-2-oxo-pyrrolidin-3- yl]ethynyl]phenyl]-4-methoxy- 3-methyl-pyridine-2-carboxamide | 380.15 |
| 170 | 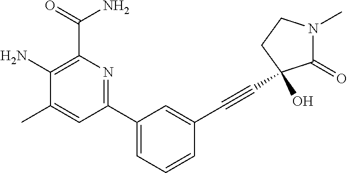 | 3-amino-6-[3-[2-[(3R)-3-hydroxy- l-methyl-2-oxo-pyrrolidin-3- yl]ethynyl]phenyl]-4-methyl- pyridine-2-carboxamide | 365.10 |
| 171 | 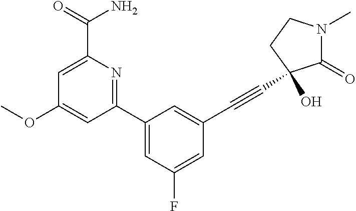 | 6-[3-fluoro-5-[2-[(3R)-3-hydroxy- 1-methyl-2-oxo-pyrrolidin-3-yl] ethynyl]phenyl]-4-methoxy- pyridine-2-carboxamide | 384.15 |
| 172 | 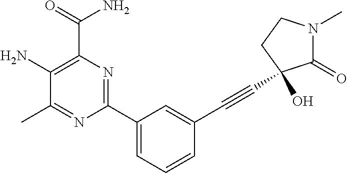 | 5-amino-2-[3-[2-[(3R)-3-hydroxy- 1-methyl-2-oxo-pyrrolidin-3-yl] ethynyl]phenyl]-6-methyl-pyrimidine- 4-carboxamide | 366.1 |
| 173 | 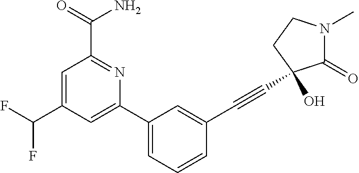 [SMILES: CN1CC[C@@](O)(C#Cc2cccc(-c3cc(C(F)F)cc(C(N)=O)n3)c2)C1=O] | 4-(difluoromethyl)-6-[3-[2-[(3R)-3- hydroxy-1-methyl-2-oxo-pyrrolidin- 3-yl]ethynyl]phenyl]pyridine-2- carboxamide | 386.10 |
| 174 | 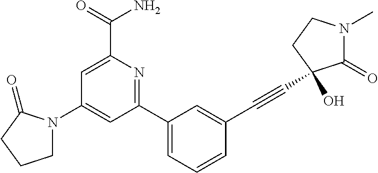 [SMILES: CN1CC[C@@](O)(C#Cc2cccc(-c3cc(N4CCCC4=O)cc(C(N)=O)n3)c2)C1=O] | 6-[3-[2-[(3R)-3-hydroxy-1- methyl-2-oxo-pyrrolidin-3- yl]ethynyl]phenyl]-4-(2- oxopyrrolidin-1-yl)pyridine-2- carboxamide | 419.1 |
| 175 | 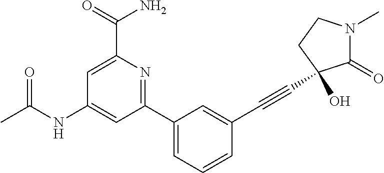 [SMILES: CC(=O)Nc1cc(C(N)=O)nc(-c2cccc(C#C[C@]3(O)CCN(C)C3=O)c2)c1] | 4-acetamido-6-[3-[2-[(3R)- 3-hydroxy-1-methyl-2-oxo- pyrrolidin-3-yl]ethynyl]phenyl] pyridine-2-carboxamide | 393.10 |
| 176 | 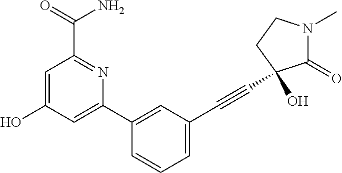 [SMILES: CN1CC[C@@](O)(C#Cc2cccc(-c3cc(O)cc(C(N)=O)n3)c2)C1=O] | 4-hydroxy-6-[3-[2-[(3R)-3- hydroxy-1-methyl-2-oxo- pyrrolidin-3-yl]ethynyl]phenyl] pyridine-2-carboxamide | 352.15 |
| 177 | 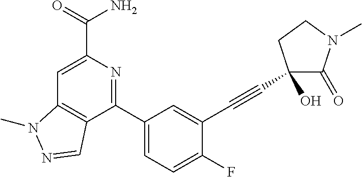 | 4-[4-fluoro-3-[2-[(3R)-3-hydroxy- l-methyl-2-oxo-pyrrolidin-3-yl] ethynyl]phenyl]-1-methyl-pyrazolo [4,3-c]pyridine-6-carboxamide | 408.15 |
| 178 | 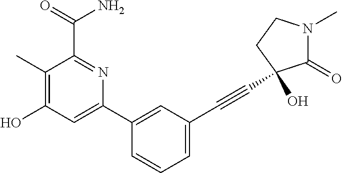 [SMILES: Cc1c(O)cc(-c2cccc(C#C[C@]3(O)CCN(C)C3=O)c2)nc1C(N)=O] | 4-hydroxy-6-[3-[2-[(3R)-3- hydroxy-1-methyl-2-oxo- pyrrolidin-3-yl]ethynyl]phenyl]- 3-methyl-pyridine-2-carboxamide | 366.10 |
| 179 | 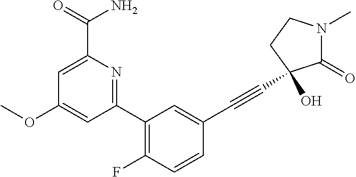 | 6-[2-fluoro-5-[2-[(3R)-3-hydroxy- l-methyl-2-oxo-pyrrolidin-3-yl] ethynyl]phenyl]-4-methoxy- pyridine-2-carboxamide | 384.10 |
| 180 | 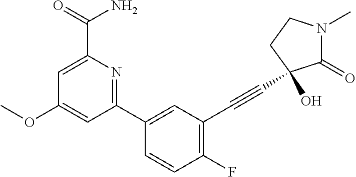 | 6-[4-fluoro-3-[2-[(3R)-3-hydroxy-1- methyl-2-oxo-pyrrolidin-3-yl] ethynyl]phenyl]-4-methoxy-pyridine- 2-carboxamide | 384.15 |
| 181 | 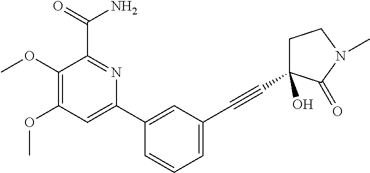 | 6-[3-[2-[(3R)-3-hydroxy-l-methyl-2- oxo-pyrrolidin-3-yl]ethynyl]phenyl]- 3,4-dimethoxy-pyridine-2- carboxamide | 396.05 |
| 182 | 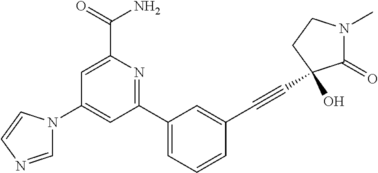 [SMILES: CN1CC[C@@](O)(C#Cc2cccc(-c3cc(-n4ccnc4)cc(C(N)=O)n3)c2)C1=O] | 6-[3-[2-[(3R)-3-hydroxy-l-methyl- 2-oxo-pyrrolidin-3-yl]ethynyl] phenyl]-4-imidazol-1-yl-pyridine- 2-carboxamide | 402.20 |
| 183 | 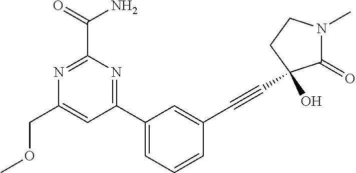 | 4-[3-[2-[(3R)-3-hydroxy-l- methyl-2-oxo-pyrrolidin-3- yl]ethynyl]phenyl]-6- (methoxymethyl)pyrimidine- 2-carboxamide | 381.1 |
| 184 | 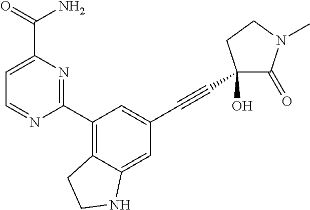 [SMILES: CN1CC[C@@](O)(C#Cc2cc3c(c(-c4nccc(C(N)=O)n4)c2)CCN3)C1=O] | 2-[6-[2-[(3R)-3-hydroxy-1- methyl-2-oxo-pyrrolidin-3- yl]ethynyl]indolin-4-yl] pyrimidine-4-carboxamide | 378.15 |
| 185 | 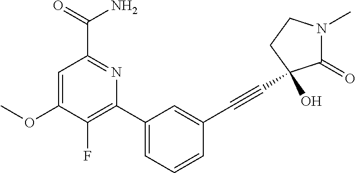 | 5-fluoro-6-[3-[2-[(3R)-3-hydroxy- 1-methyl-2-oxo-pyrrolidin- 3-yl]ethynyl]phenyl]-4-methoxy- pyridine-2-carboxamide | 384.15 |
| 186 | 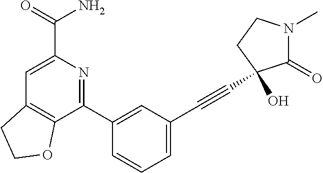 [SMILES: CN1CC[C@@](O)(C#Cc2cccc(-c3nc(C(N)=O)cc4c3OCC4)c2)C1=O] | 7-[3-[2-[(3R)-3-hydroxy-1- methyl-2-oxo-pyrrolidin-3- yl]ethynyl]phenyl]-2,3- dihydrofuro[1,3-c]pyridine-5- carboxamide | 378.10 |
| 187 | 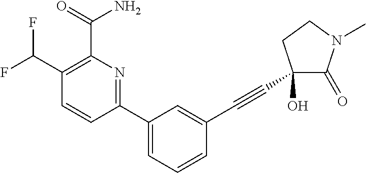 [SMILES: CN1CC[C@@](O)(C#Cc2cccc(-c3ccc(C(F)F)c(C(N)=O)n3)c2)C1=O] | 3-(difluoromethyl)-6-[3-[2- [(3R)-3-hydroxy-1-methyl- 2-oxo-pyrrolidin-3-yl]ethynyl] phenyl]pyridine-2-carboxamide | 386.1 |
| 188 | 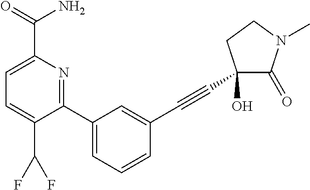 [SMILES: CN1CC[C@@](O)(C#Cc2cccc(-c3nc(C(N)=O)ccc3C(F)F)c2)C1=O] | 5-(difluoromethyl)-6-[3[2- [(3R)-3-hydroxy-1-methyl- 2-oxo-pyrrolidin-3-yl]ethynyl] phenyl]pyridine-2-carboxamide | 386.1 |
| 189 | 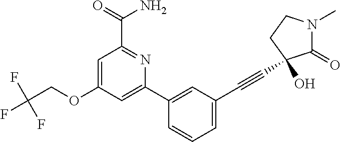 [SMILES: CN1CC[C@@](O)(C#Cc2cccc(-c3cc(OCC(F)(F)F)cc(C(N)=O)n3)c2)C1=O] | 6-[3-[2-[(3R)-3-hydroxy-1- methyl-2-oxo-pyrrolidin-3- yl]ethynyl]phenyl]-4(2,2,2- trifluoroethoxy)pyridine-2- carboxamide | 434.3 |
| 190 | 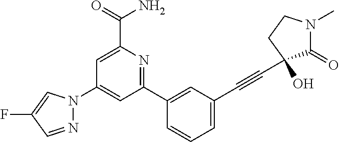 [SMILES: CN1CC[C@@](O)(C#Cc2cccc(-c3cc(-n4cc(F)cn4)cc(C(N)=O)n3)c2)C1=O] | 4-(4-fluoropyrazol-1-yl)-6- [3-[2-[(3R)-3-hydroxy-1-methyl- 2-oxo-pyrrolidin-3-yl]ethynyl] phenyl]pyridine-2-carboxamide | 420.10 |
| 191 | 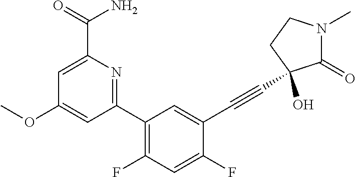 | 6[2,4-difluoro-5-[2-[(3R)- 3-hydroxy-l-methy1-2-oxo- pyrrolidin-3-yl]ethynyl]phenyl]- 4-methoxy-pyridine-2- carboxamide | 402.10 |
| 192 | 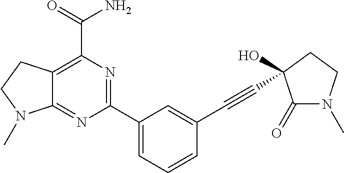 [SMILES: CN1CC[C@@](O)(C#Cc2cccc(-c3nc(C(N)=O)c4c(n3)N(C)CC4)c2)C1=O] | 2-[3-[2-[(3R)-3-hydroxy-1- methyl-2-oxo-pyrrolidin-3- yl]ethynyl]phenyl]-7-methyl- 5,6-dihydropyrrolo[2,3-d] pyrimidine-4-carboxamide | 392.15 |
| 193 | 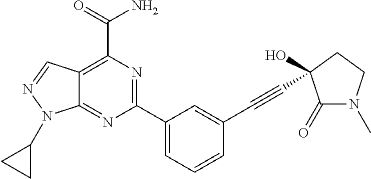 [SMILES: CN1CC[C@@](O)(C#Cc2cccc(-c3nc(C(N)=O)c4cnn(C5CC5)c4n3)c2)C1=O] | 1-cyclopropyl-6-[3-[2-[(3R)- 3-hydroxy-1-methyl-2-oxo- pyrrolidin-3-yl]ethynyl] phenyl]pyrazolo[3,4-d] pyrimidine-4-carboxamide | 417.15 |
| 194 | 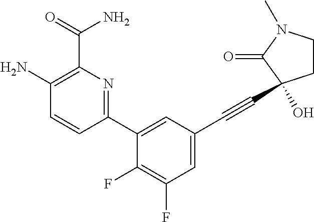 [SMILES: CN1CC[C@@](O)(C#Cc2cc(F)c(F)c(-c3ccc(N)c(C(N)=O)n3)c2)C1=O] | 3-amino-6-[2,3-difluoro-5- [2-[(3R)-3-hydroxy-l-methyl- 2-oxo-pyrrolidin-3-yl] ethynyl]phenyl]pyridine-2- carboxamide | 387.1 |
| 195 | 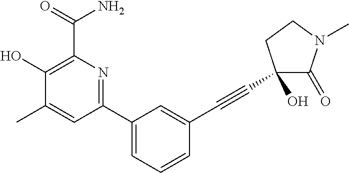 | 3-hydroxy-6-[3-[2-[(3R)-3- hydroxy-1-methyl-2-oxo- pyrrolidin-3-yl]ethynyl]phenyl]- 4-methyl-pyridine-2-carboxamide | 366.10 |
| 196 | 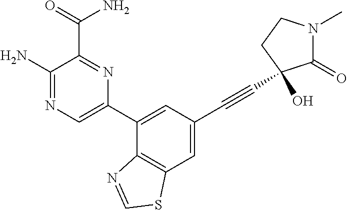 [SMILES: CN1CC[C@@](O)(C#Cc2cc(-c3cnc(N)c(C(N)=O)n3)c3ncsc3c2)C1=O] | 3-amino-6-[6-[2-[(3R)-3-hydroxy- 1-methyl-2-oxo-pyrrolidin- 3-yl]ethynyl]-1,3-benzothiazol- 4-yl]pyrazine-2-carboxamide | 409.10 |
| 197 | 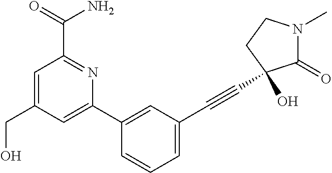 [SMILES: CN1CC[C@@](O)(C#Cc2cccc(-c3cc(CO)cc(C(N)=O)n3)c2)C1=O] | 4-(hydroxymethyl)-6-[3-[2- [(3R)-3-hydroxy-1-methyl- 2-oxo-pyrrolidin-3-yl]ethynyl] phenyl]pyridine-2-carboxamide | 366.1 |
| 198 | 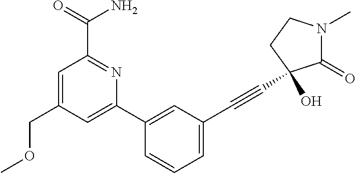 | 6-[3-[2-[(3R)-3-hydroxy-1- methyl-2-oxo-pyrrolidin-3- yl]ethynyl]phenyl]-4- (methoxymethyl)pyridine-2- carboxamide | 380.10 |
| 199 | 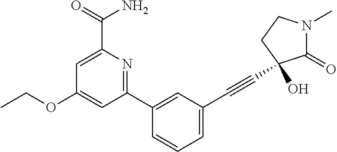 [SMILES: CCOc1cc(C(N)=O)nc(-c2cccc(C#C[C@]3(O)CCN(C)C3=O)c2)c1] | 4-ethoxy-6-[3-[2-[(3R)-3-hydroxy- 1-methyl-2-oxo-pyrrolidin-3-yl] ethynyl]phenyl]-4-(methoxymethyl) pyridine-2-carboxamide | 380.1 |
| 200 | 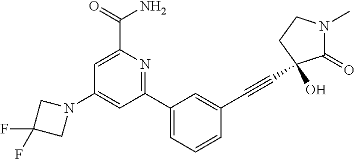 [SMILES: CN1CC[C@@](O)(C#Cc2cccc(-c3cc(N4CC(F)(F)C4)cc(C(N)=O)n3)c2)C1=O] | 4-(3,3-difluoroazetidin-1-yl)- 6-[3-[2-[(3R)-3-hydroxy- 1-methyl-2-oxo-pyrrolidin- 3-yl]ethynyl]phenyl]pyridine- 2-carboxamide | 427.15 |

NIK Enzyme Inhibition Assay:

The ability of the nuclear factor-kappa B (NF-kB)-inducing kinase (NIK) to catalyze the hydrolysis of adenosine-5′-triphosphate (ATP) was monitored using the Transcreener ADP (adenosine-5′-diphosphate) assay (BellBrook Labs). Purified NIK (0.5 nM) derived from a baculovirus-infected insect cell expression system was incubated with test compounds for 1-3.5 hours in 50 mM 2-[4-(2-hydroxyethyl)piperazin-1-yl]ethanesulfonic acid buffer (pH 7.2) containing 10 mM MgCl₂, 2 mM dithiothreitol, 10 μM ATP, 0.01% Triton X-100, 0.1% gamma-globulins from bovine blood, 1% dimethylsulfoxide (DMSO), 7 μg/mL ADP antibody and 5 nM ADP-MR121 633 tracer. Reactions were quenched by the addition of 20 mM 2,2′,2″,2′″-(ethane-1,2-diyldinitrilo)tetraacetic acid and 0.01% Brij 35. The tracer bound to the antibody was displaced by the ADP generated during the NIK reaction, which causes a decrease in fluorescence polarization that was measured by laser excitation at 633 nm with a Fluorescence Correlation Spectroscopy Plus reader (Evotec AG). Equilibrium dissociation constant (Kᵢ) values for NIK inhibitors are calculated from plots of activity vs. inhibitor concentration using Morrison's quadratic equation that accounts for the potential of tight binding, and by also applying the conversion factor that accounted for competitive inhibition and the concentration of substrate used in the assay relative to its Michaelis constant (Kₘ). The compounds in listed in Table 1 have the corresponding inhibitory value (NIK ADP-FP, Kᵢ in micromolar) for NIK described in Table 2 below.

Cellular Assay:

Several assays were developed to profile the cellular activities of NIK inhibitors.

(1) The first assay that can be used to profile whether a test compound can inhibit the NF-kB signal through NIK inhibition without affecting cell viability. In this assay, human embryonic kidney 293 cells are stably transfected with a tetracycline-inducible NIK DNA construct containing a cytomegalovirus promoter plus two reporter DNA constructs. One reporter encodes firefly luciferase under the control of three repeats of an NF-kB response element from the ELAM-1 gene and reflects the level of NIK activity in the cells, whereas the other reporter constitutively expresses Renilla luciferase under the control of the herpes simplex virus thymidine kinase promoter and serves as a general measure of cell viability. Cells are incubated with different concentrations of compounds (0.2% DMSO final) in medium containing 1 μg/mL doxycycline and 10% tet-system approved fetal bovine serum (Clontech) for 24 hours, after which the reporters' signals are detected using the Dual Glo luciferase detection system (Promega) according to the vendor's protocol.

(2) A second set of cell assay are used to define the selectivity of NIK inhibitors toward inhibition of classical vs. non-classical NF-kB signaling and rely on quantification of the nuclear translocation of p52 (NF-kB2) and REL-A (p65) using high content cellular imaging. For the p52 (non-classical NF-kB signaling) nuclear translocation assay, HeLa cells are treated with different concentrations of compounds (0.2% DMSO final) in medium containing 10% fetal bovine serum and then stimulated with 100 ng/mL of an anti-lymphotoxin beta receptor antibody (R&D Systems) for 5 hours. In the REL-A nuclear translocation assay, HeLa cells are incubated with compounds (0.2% DMSO final) for 4.5 hours in medium containing 10% fetal bovine serum before stimulating them with 10 ng/mL tumor necrosis factor (TNF)-α (R&D Systems) for 30 minutes. Cells are fixed with 4% paraformaldehyde, permeabilized by adding 0.1% Triton X-100 in phosphate buffered saline, and then are incubated with either 2 ug/mL anti-p52 antibody (Millipore) or 400 ng/mL anti-REL-A (p65) antibody (Santa Cruz Biotechnology). Finally, the cells are incubated with an Alexa488-labeled secondary antibody (Invitrogen) and DRAQ5 DNA stain (Biostatus). Imaging is carried out using an Opera reader (Perkin Elmer) and data are analyzed with the aid of Acapella software (Perkin Elmer). The p52 or REL-Atranslocation into the nucleus is quantified by the ratio of the nuclear to cytoplasmic signal intensity. The concentration of inhibitor required for 50% inhibition (IC₅₀ values) in these cell assays are derived from the plots of signal vs. inhibitor concentration. The compounds in listed in Table 1 have the corresponding inhibitory value (IC₅₀ in micromolar) for NIK p52 Translocation Assay as set forth in Table 2.

The compounds in listed in Table 1 have the corresponding inhibitory values (IC₅₀ in micromolar) for the Translocation Assays as set forth in Table 2.

TABLE 2

| | | REL-A | p52 |
|---|---|---|---|
| | | HeLa | HeLa |
| | | Transloc | Transloc |
| | NIK | Assay | Assay |
| | ADP-FP | (IC₅₀) | (IC₅₀) |
| Ex. | (Kᵢ μM) | [μM] | [μM] |
| 1 | 0.00026 | >20 | 0.0932 |
| 2 | 0.00173 | >20 | 0.101 |
| 3 | 0.00282 | >20 | 0.674 |
| 4 | 0.000050 | >20 | 0.0319 |
| 5 | 0.00080 | >20 | 0.122 |
| 6 | 0.00083 | >20 | 0.108 |
| 7 | 0.00125 | | |
| 8 | 0.00235 | >20 | 0.211 |
| 9 | 0.00124 | >20 | 0.27 |
| 10 | 0.00117 | >20 | 0.266 |
| 11 | 0.00465 | >20 | 2.5 |
| 12 | 0.00033 | >20 | 0.236 |
| 13 | 0.00048 | >20 | 0.382 |
| 14 | 0.00038 | >20 | 0.665 |
| 15 | 0.00121 | >20 | 0.63 |
| 16 | 0.00054 | >20 | 0.191 |
| 17 | 0.00084 | >20 | 0.225 |
| 18 | 0.00219 | >20 | 0.039 |
| 19 | 0.0011 | >20 | 0.0455 |
| 20 | 0.00079 | 19.1 | 0.374 |
| 21 | 0.0012 | >20 | 0.0842 |
| 22 | 0.00114 | >20 | 0.085 |
| 23 | 0.00148 | >20 | 0.0961 |
| 24 | 0.00105 | | |
| 25 | 0.00557 | | |
| 26 | 0.00485 | >20 | 0.11 |
| 27 | 0.00334 | >20 | 0.0872 |
| 28 | 0.00238 | >20 | 0.229 |
| 29 | 0.00029 | >20 | 0.0466 |
| 30 | 0.0232 | | |
| 31 | 0.00093 | >20 | 0.479 |
| 32 | 0.00022 | >20 | 0.244 |
| 33 | <0.000050 | >28 | 0.0363 |
| 34 | 0.00142 | >20 | 0.138 |
| 35 | 0.00223 | >20 | 0.386 |
| 36 | 0.000184 | >20 | 0.314 |
| 37 | 0.00012+ | >20 | 0.0139 |
| 38 | 0.00019 | >20 | 0.0491 |
| 39 | 0.00013 | 7.7 | 0.0209 |
| 40 | <0.000050 | >8 | 0.0078 |
| 41 | 0.00022 | >20 | 0.0264 |
| 42 | 0.00010 | >20 | 0.0109 |
| 43 | 0.000080 | >20 | 0.0141 |
| 44 | 0.00011 | >20 | 0.0175 |
| 45 | 0.00039 | >20 | 0.122 |
| 46 | 0.00062 | >20 | 0.103 |
| 47 | 0.00094 | 20.0 | 0.27 |
| 48 | 0.00039 | 20.0 | 0.122 |
| 49 | 0.000050 | 2.0 | 0.0082 |
| 50 | 0.00108 | 20.0 | 0.346 |
| 51 | 0.00023 | 20.0 | 0.129 |
| 52 | 0.00013 | 20.0 | 0.026 |
| 53 | 0.00067 | 20.0 | 0.725 |
| 54 | 0.51 | | |
| 55 | 1.25 | | |
| 56 | 1.25 | | |
| 57 | 1.25 | | |
| 58 | 1.25 | | |
| 59 | 0.00016 | 6.32 | 0.0232 |
| 60 | 0.00561 | 20.0 | 1.39 |
| 61 | 0.00433 | | |
| 62 | 0.00011 | 1.0 | 0.0115 |
| 63 | 0.000080 | 20.0 | 0.0107 |
| 64 | 0.00407 | | |
| 65 | 0.038 | | |
| 66 | 0.00016 | 20.0 | 0.139 |
| 67 | 0.000080 | 20.0 | 0.0218 |
| 68 | 0.00053 | 20.0 | 0.0894 |
| 69 | 0.00060 | 20.0 | 0.206 |
| 70 | 0.00094 | 20.0 | 0.079 |
| 71 | 0.00018 | 20.0 | 0.00894 |
| 72 | 0.000050 | 6.32 | 0.00643 |
| 73 | 0.0031 | 20.0 | 0.837 |
| 74 | 0.00053 | 20.0 | 0.0602 |
| 75 | 0.00038 | 20.0 | 0.0668 |
| 76 | 0.00086 | 20.0 | 0.101 |
| 77 | 0.00099 | 20.0 | 0.247 |
| 78 | 0.00010 | 15.7 | 0.0083 |
| 79 | 0.000050 | 20.0 | 0.00986 |
| 80 | 0.000050 | 20.0 | 0.00465 |
| 81 | 0.000050 | 2.0 | 0.0508 |
| 82 | 0.00033 | 2.0 | 0.0955 |
| 83 | 0.00015 | 1.0 | 0.011 |
| 84 | 0.000050 | 1.0 | 0.0142 |
| 85 | 0.000050 | 6.32 | 0.00588 |
| 86 | 0.00010 | 20.0 | 0.00627 |
| 87 | 0.00014 | 1.0 | 0.0040 |
| 88 | 0.000050 | 20.0 | 0.00212 |
| 89 | 0.000050 | 1.0 | 0.0324 |
| 90 | 0.00035 | 20.0 | 0.0168 |
| 91 | 0.0144 | | |
| 92 | 0.00039 | 20.0 | 0.133 |
| 93 | 0.00015 | 6.32 | 0.021 |
| 94 | 0.00141 | 20.0 | 0.175 |
| 95 | 0.00049 | 20.0 | 0.0552 |
| 96 | 0.00045 | 20.0 | 0.0347 |
| 97 | 0.00037 | 20.0 | 0.0575 |
| 98 | 0.00023 | 20.0 | 0.082 |
| 99 | 0.00048 | 20.0 | 0.104 |
| 100 | 0.00010 | 20.0 | 0.0116 |
| 101 | 0.00052 | 20.0 | 0.0926 |
| 102 | 0.00021 | 20.0 | 0.0669 |
| 103 | 0.0228 | | |
| 104 | 0.00040 | 20.0 | 0.0692 |
| 105 | 0.00035 | 2.0 | 0.0733 |
| 106 | 0.00025 | 2.0 | 0.070 |
| 107 | 0.00040 | 2.0 | 0.269 |
| 108 | 0.00065 | 2.0 | 0.213 |
| 109 | 0.00028 | 2.0 | 0.0831 |
| 110 | 0.00014 | 2.0 | 0.0843 |
| 111 | 0.00037 | 2.0 | 0.0758 |
| 112 | 0.000050 | 20.0 | 0.0888 |
| 113 | 0.00212 | 2.0 | 0.212 |
| 114 | 0.00148 | 2.0 | 0.211 |
| 115 | 0.00177 | | |
| 116 | 0.000050 | 12.4 | 0.0116 |
| 117 | 0.000050 | 20.0 | 0.0125 |
| 118 | 0.000050 | 2.0 | 0.019 |
| 119 | 0.00010 | 2.0 | 0.108 |
| 120 | 0.00033 | 2.0 | 0.0266 |
| 121 | 0.0676 | | |
| 122 | 0.00081 | 2.0 | 0.226 |
| 123 | 0.000090 | 2.0 | 0.0794 |
| 124 | 0.000050 | 2.0 | 0.30 |
| 125 | 0.00129 | 2.0 | 0.0842 |
| 126 | 0.000050 | 2.0 | 0.122 |
| 127 | 0.000080 | 2.0 | 0.183 |
| 128 | 0.000050 | 2.0 | 0.0272 |
| 129 | 0.00030 | 2.0 | 0.155 |
| 130 | 0.00905 | 2.0 | 2.0 |
| 131 | 0.00106 | 2.0 | 0.142 |
| 132 | 0.00875 | 2.0 | 2.0 |
| 133 | 0.00018 | 2.0 | 0.16 |
| 134 | 0.000090 | 2.0 | 0.0454 |
| 135 | 0.00053 | 2.0 | 0.147 |
| 136 | 0.00050 | 2.0 | 0.152 |
| 137 | 0.00368 | 20.0 | 0.268 |
| 138 | 0.0602 | | |
| 139 | 0.000070 | 2.0 | 0.0222 |
| 140 | 0.0238 | | |
| 141 | 0.00037 | 20.0 | 0.328 |
| 142 | 0.00024 | 20.0 | 0.0148 |
| 143 | 0.000050 | 20.0 | 0.00812 |
| 144 | 0.00069 | 2.0 | 0.199 |
| 145 | 0.000050 | 2.0 | 0.038 |
| 146 | 0.00037 | 20.0 | 0.0116 |
| 147 | 0.879 | | |
| 148 | 0.00019 | 2.0 | 0.0524 |
| 149 | 0.00088 | 2.0 | 0.0511 |
| 150 | 0.00099 | >2 | 0.628 |
| 151 | 0.00067 | >2 | 0.396 |
| 152 | <0.000050 | >2 | 0.046 |
| 153 | 0.00010 | >2 | 0.045 |
| 154 | 0.00093 | >2 | 0.40 |
| 155 | <0.000050 | >2 | 0.277 |
| 156 | 0.0113 | | |
| 157 | 0.00186 | | |
| 158 | 0.000060 | >2 | 0.063 |
| 159 | <0.000050 | >2 | 0.073 |
| 160 | 0.00050 | >2 | 0.416 |
| 161 | 0.00033 | >2 | 0.445 |
| 162 | <0.000050 | >2 | 0.067 |
| 163 | 0.00021 | | |
| 164 | <0.000050 | >2 | 0.062 |
| 165 | 0.000080 | >2 | 0.18 |
| 166 | <0.000050 | >2 | 0.059 |
| 167 | <0.000050 | >2 | 0.085 |
| 168 | 0.00016 | >2 | 0.13 |
| 169 | 0.00055 | >2 | 0.196 |
| 170 | <0.00010 | >2 | 0.026 |
| 171 | 0.00024 | >2 | 0.132 |
| 172 | 0.00036 | >2 | 0.078 |
| 173 | 0.00031 | >2 | 0.108 |
| 174 | 0.00053 | >2 | 0.449 |
| 175 | 0.000080 | | |
| 176 | 0.00096 | | |
| 177 | <0.000050 | | |
| 178 | 0.00287 | | |
| 179 | <0.000050 | | |
| 180 | <0.000050 | | |
| 181 | 0.00367 | | |
| 182 | 0.00017 | | |
| 183 | 0.00049 | | |
| 184 | 0.00014 | | |
| 185 | 0.00050 | | |
| 186 | 0.00014 | | |
| 187 | 0.000060 | | |
| 188 | 0.00953 | | |
| 189 | 0.00015 | | |
| 190 | <0.000050 | | |
| 191 | <0.000050 | | |
| 192 | 0.0004 | | |
| 193 | <0.0001 | | |
| 194 | <0.00005 | | |
| 195 | <0.00005 | | |
| 196 | 0.001 | | |
| 197 | 0.0008 | | |
| 198 | 0.0005 | | |
| 199 | 0.0004 | | |
| 200 | <0.00005 | | |

Blank = not determined

What is claimed is:

1. A compound selected from the following: 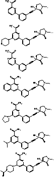 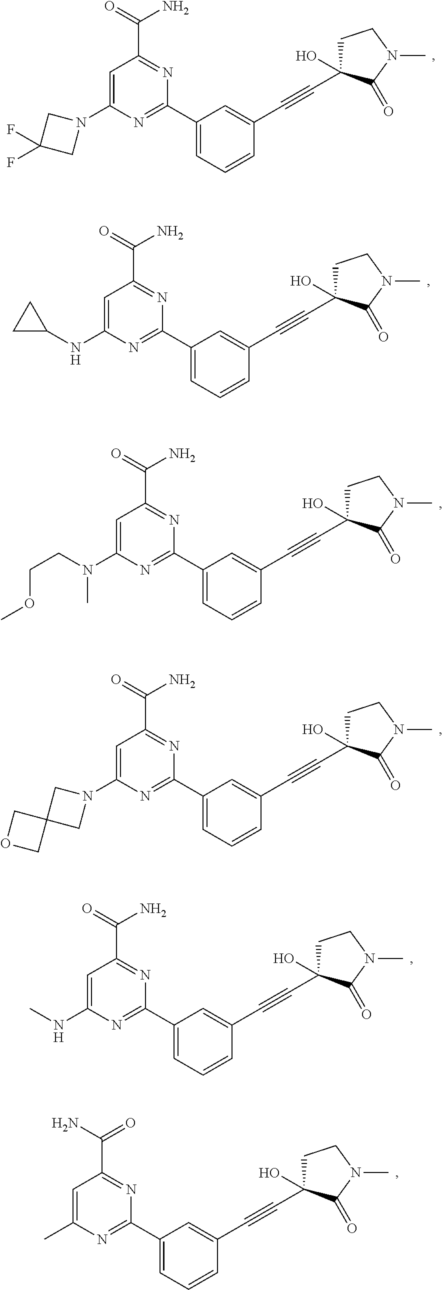 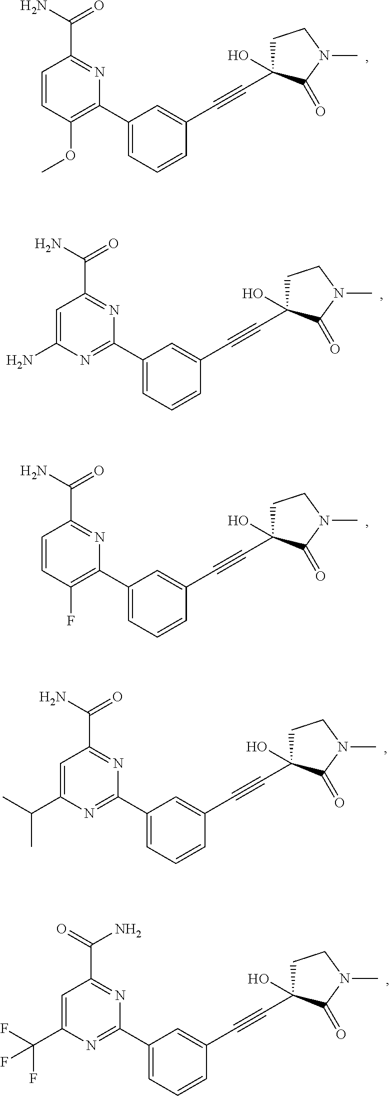 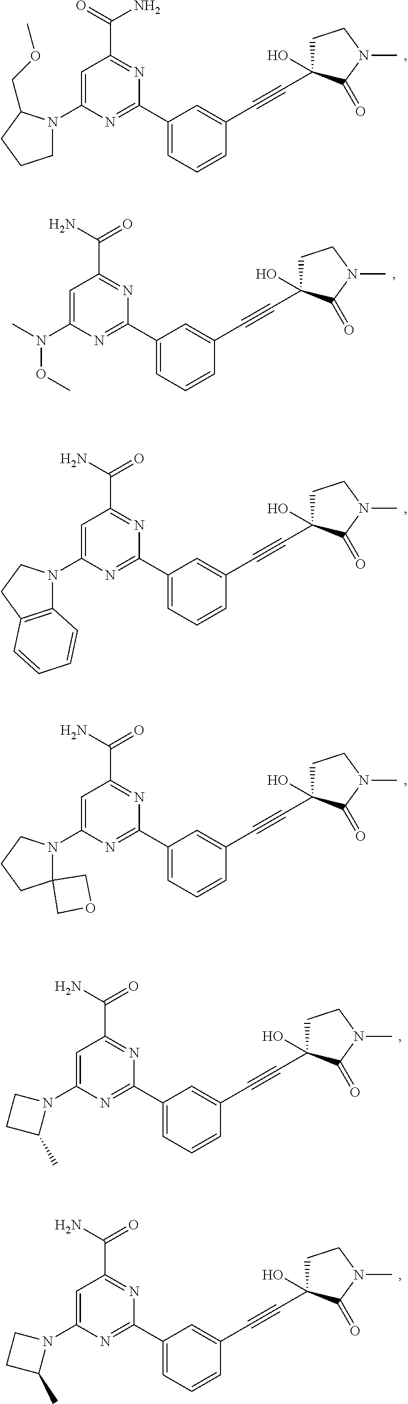 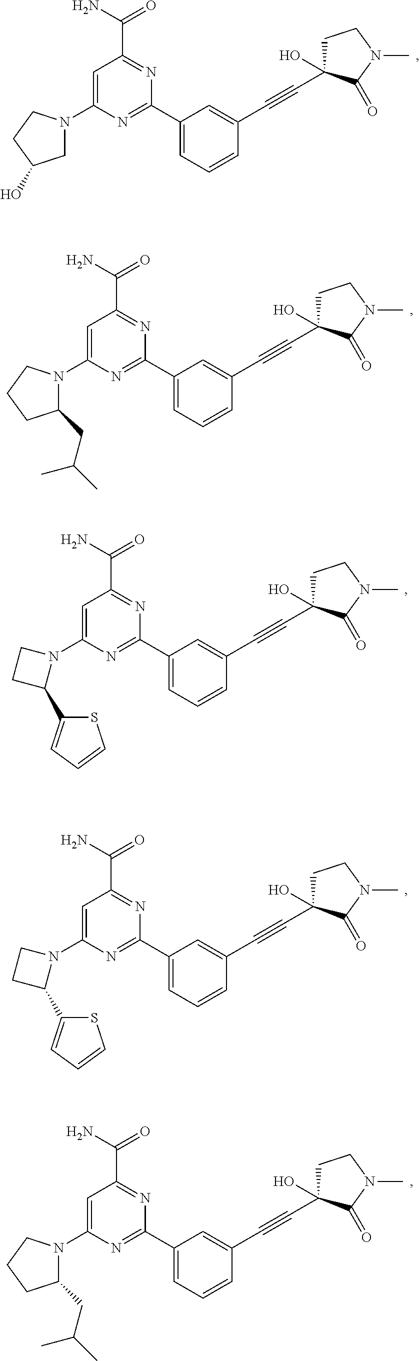 [SMILES: CN1CC[C@@](O)(C#Cc2cccc(-c3nc(C(N)=O)cc(N4CC[C@@H](O)C4)n3)c2)C1=O ; CC(C)C[C@H]1CCCN1c1cc(C(N)=O)nc(-c2cccc(C#C[C@]3(O)CCN(C)C3=O)c2)n1 ; CN1CC[C@@](O)(C#Cc2cccc(-c3nc(C(N)=O)cc(N4CC[C@@H]4c4cccs4)n3)c2)C1=O ; CN1CC[C@@](O)(C#Cc2cccc(-c3nc(C(N)=O)cc(N4CC[C@H]4c4cccs4)n3)c2)C1=O ; CC(C)C[C@@H]1CCCN1c1cc(C(N)=O)nc(-c2cccc(C#C[C@]3(O)CCN(C)C3=O)c2)n1] 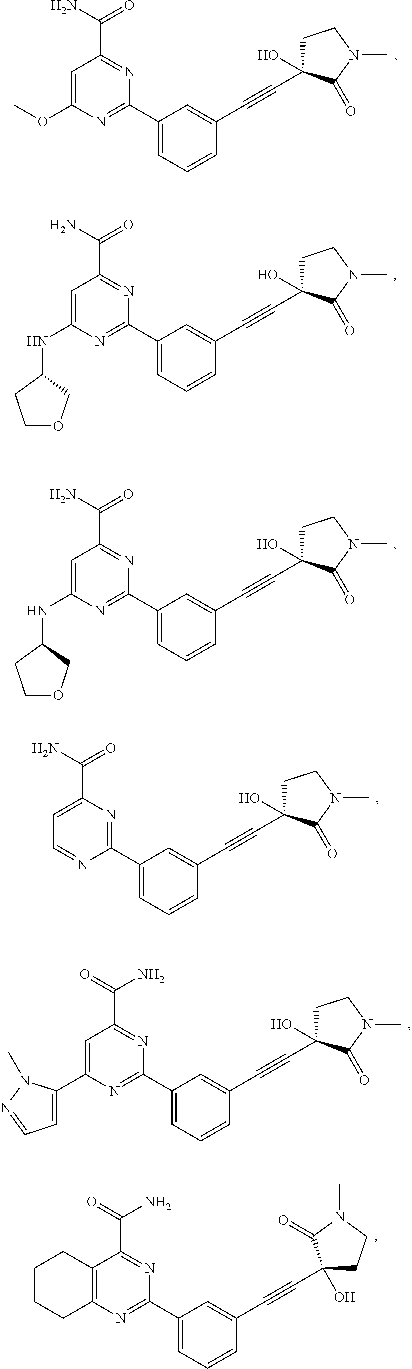 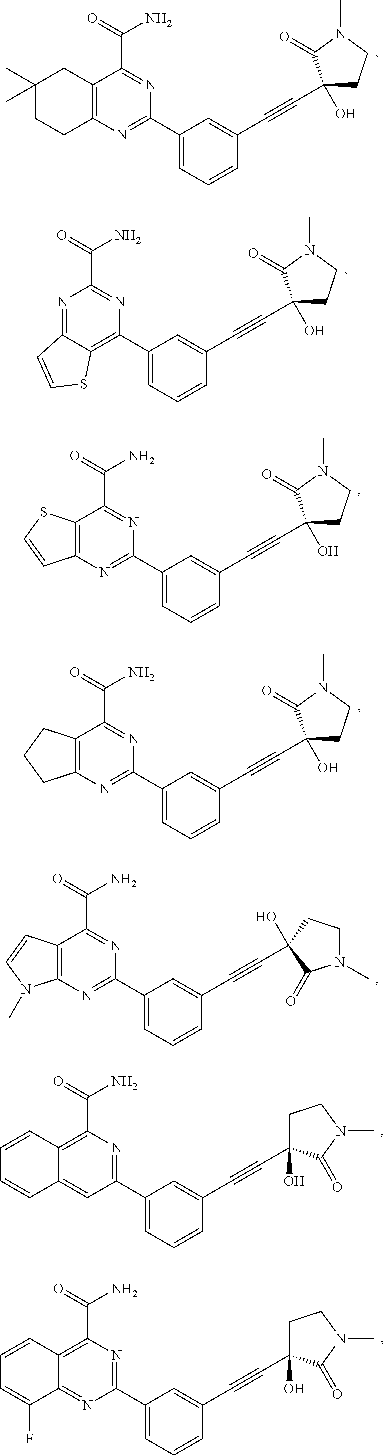 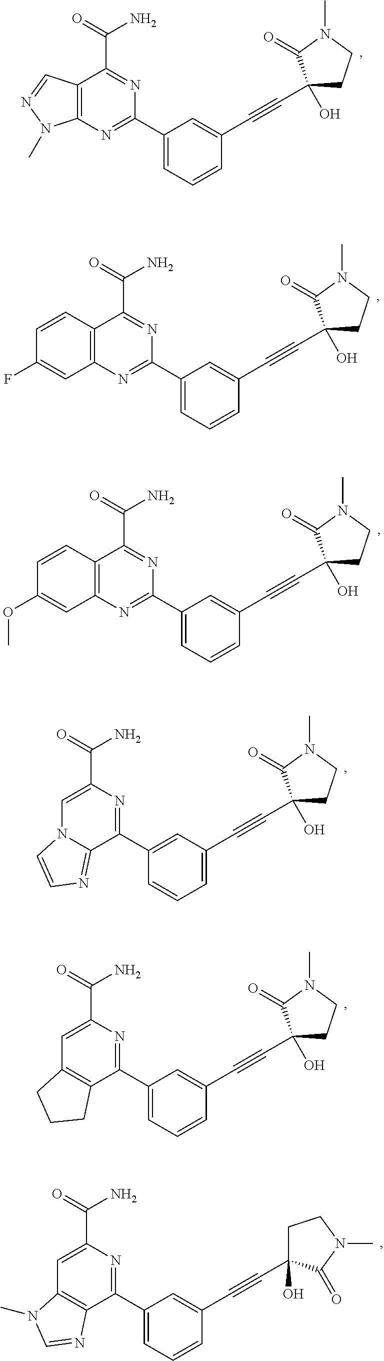 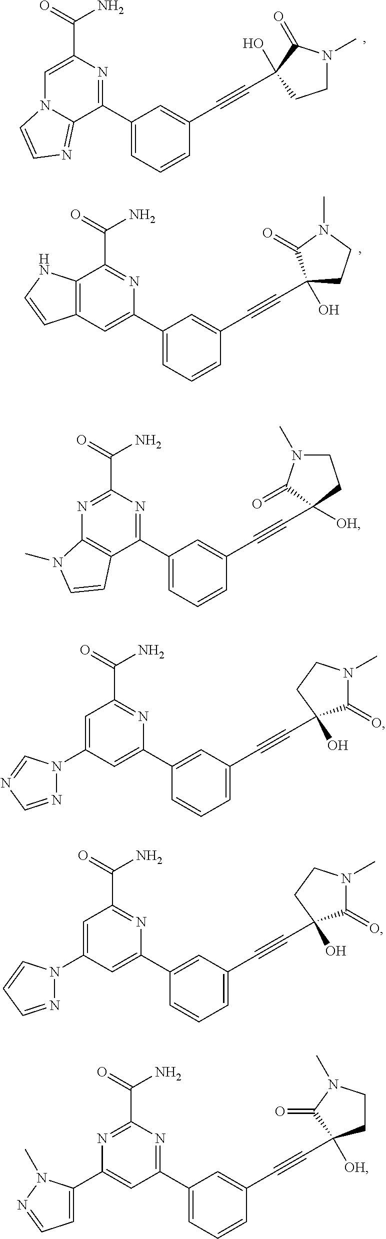 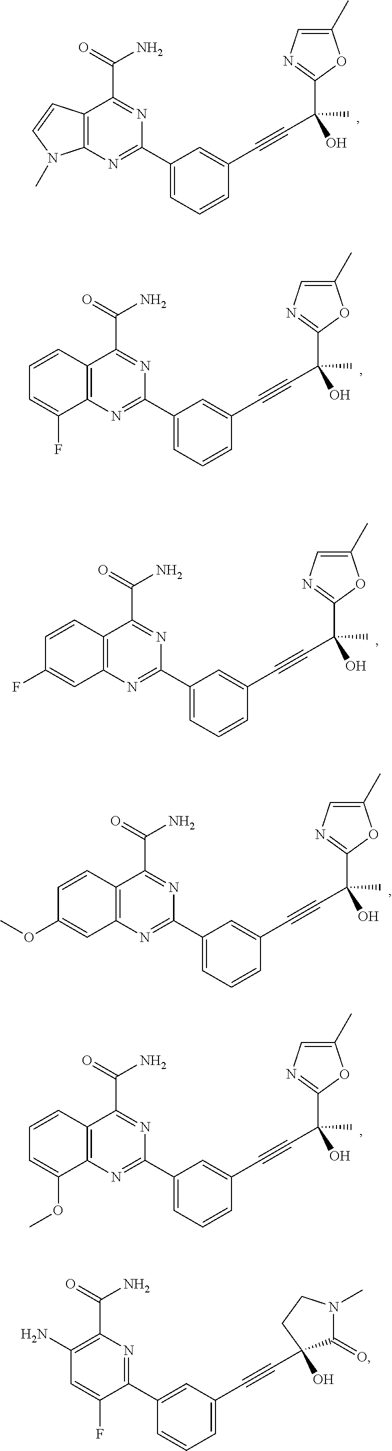 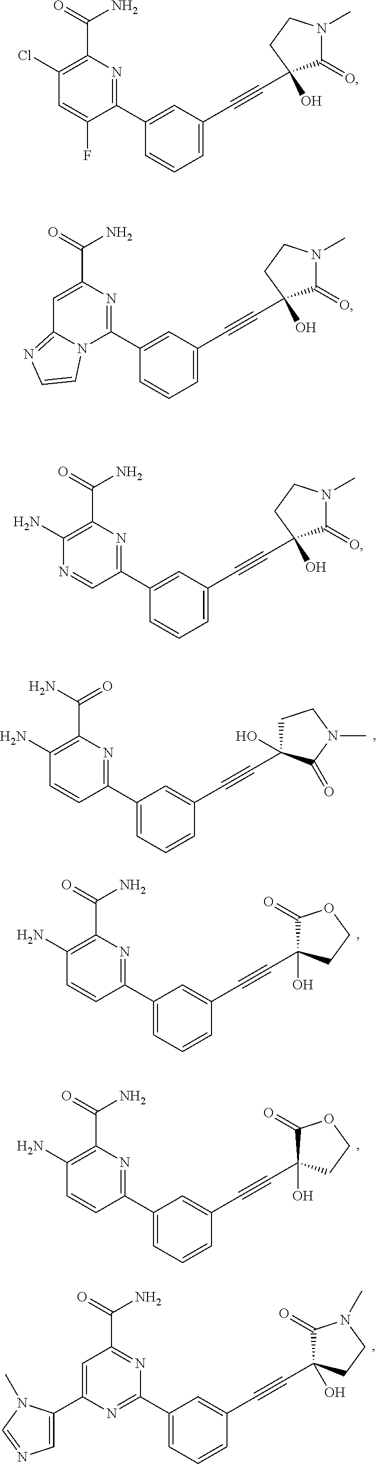 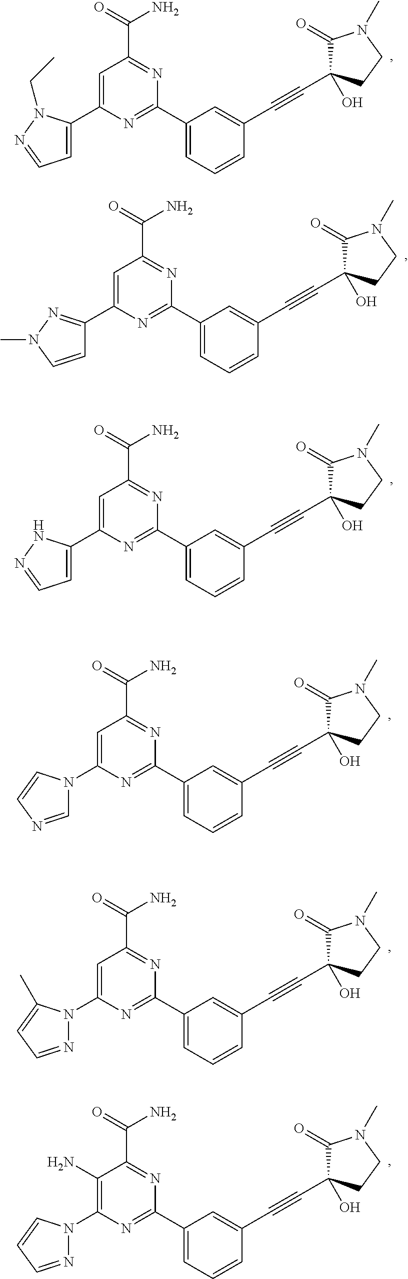 [SMILES: CCn1nccc1-c1cc(C(N)=O)nc(-c2cccc(C#C[C@]3(O)CCN(C)C3=O)c2)n1 ; CN1CC[C@@](O)(C#Cc2cccc(-c3nc(C(N)=O)cc(-c4ccn(C)n4)n3)c2)C1=O ; CN1CC[C@@](O)(C#Cc2cccc(-c3nc(C(N)=O)cc(-c4ccn[nH]4)n3)c2)C1=O ; CN1CC[C@@](O)(C#Cc2cccc(-c3nc(C(N)=O)cc(-n4ccnc4)n3)c2)C1=O ; Cc1ccnn1-c1cc(C(N)=O)nc(-c2cccc(C#C[C@]3(O)CCN(C)C3=O)c2)n1 ; CN1CC[C@@](O)(C#Cc2cccc(-c3nc(C(N)=O)c(N)c(-n4cccn4)n3)c2)C1=O] 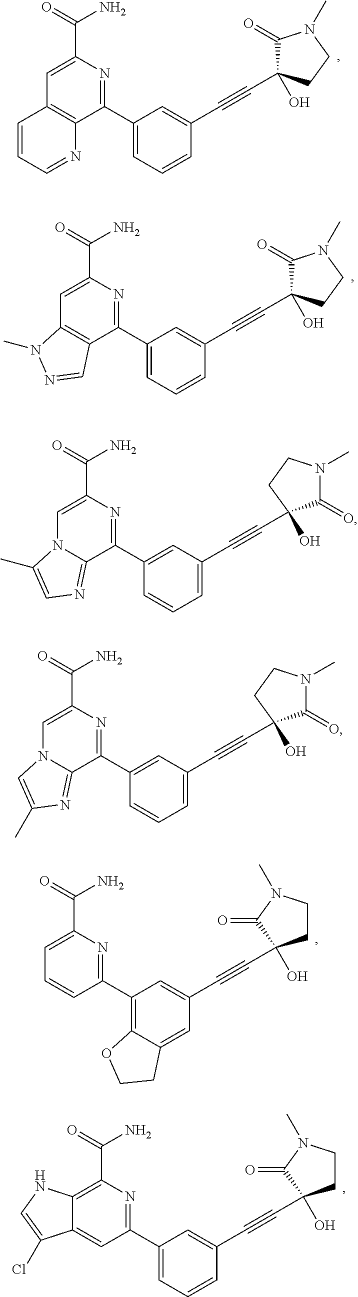 [SMILES: CN1CC[C@@](O)(C#Cc2cccc(-c3nc(C(N)=O)cc4cccnc34)c2)C1=O ; CN1CC[C@@](O)(C#Cc2cccc(-c3nc(C(N)=O)cc4c3cnn4C)c2)C1=O ; Cc1cnc2c(-c3cccc(C#C[C@]4(O)CCN(C)C4=O)c3)nc(C(N)=O)cn12 ; Cc1cn2cc(C(N)=O)nc(-c3cccc(C#C[C@]4(O)CCN(C)C4=O)c3)c2n1 ; CN1CC[C@@](O)(C#Cc2cc3c(c(-c4cccc(C(N)=O)n4)c2)OCC3)C1=O ; CN1CC[C@@](O)(C#Cc2cccc(-c3cc4c(Cl)c[nH]c4c(C(N)=O)n3)c2)C1=O] 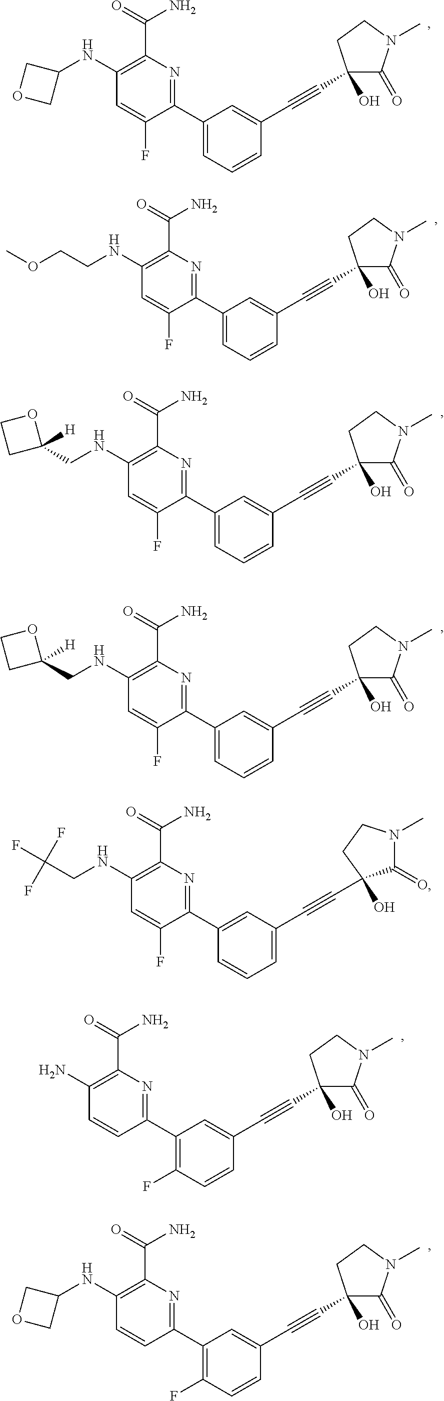 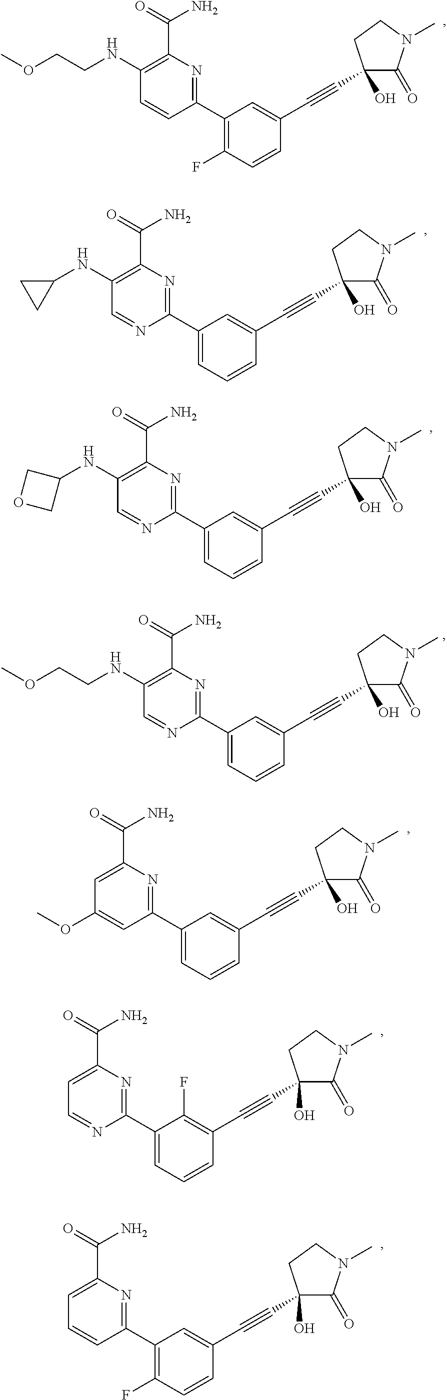 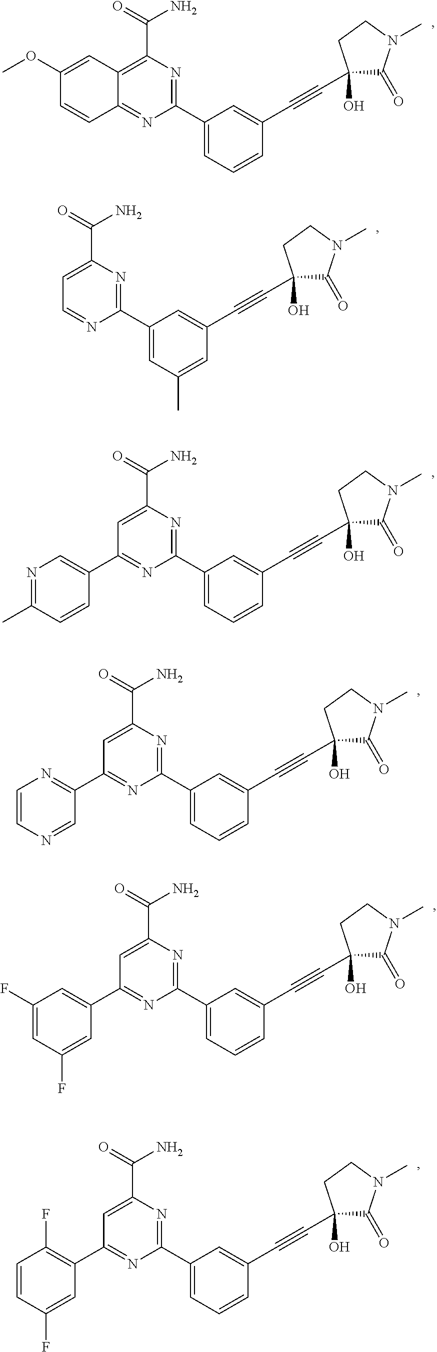 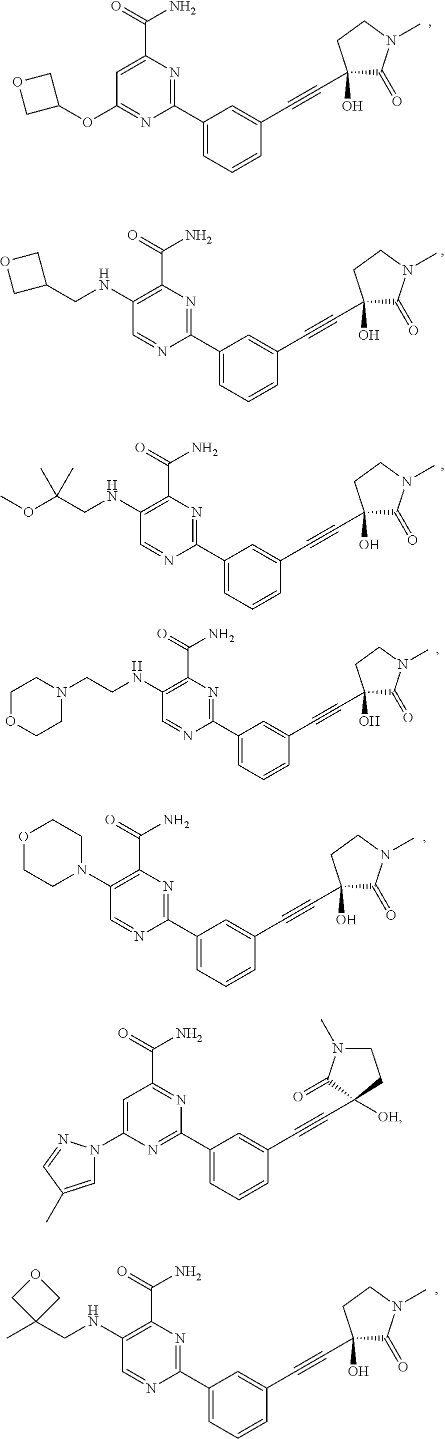 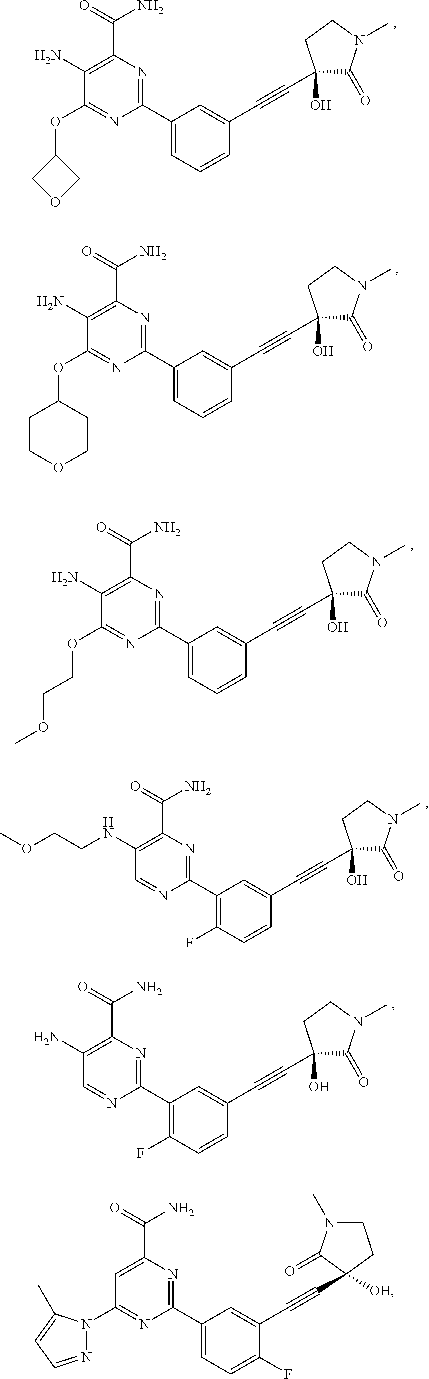 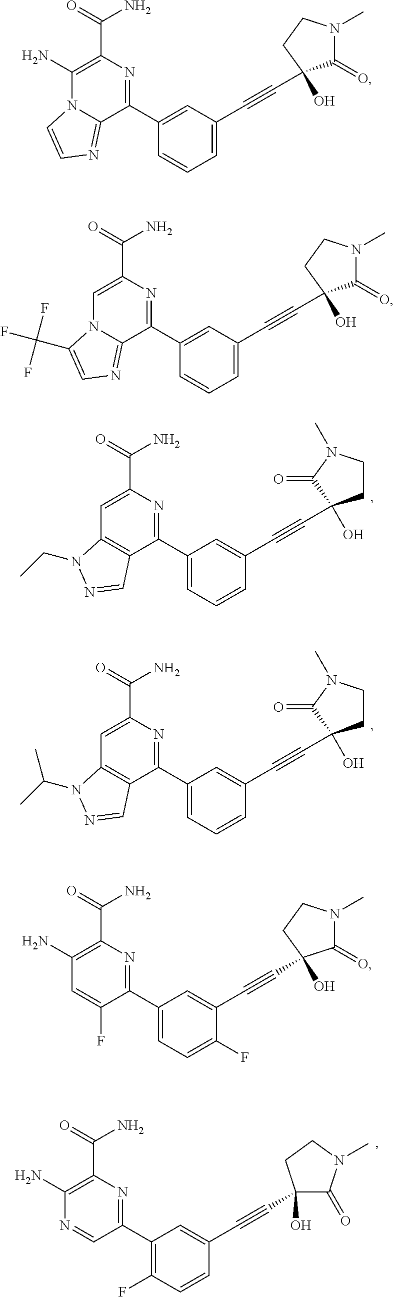 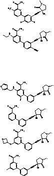 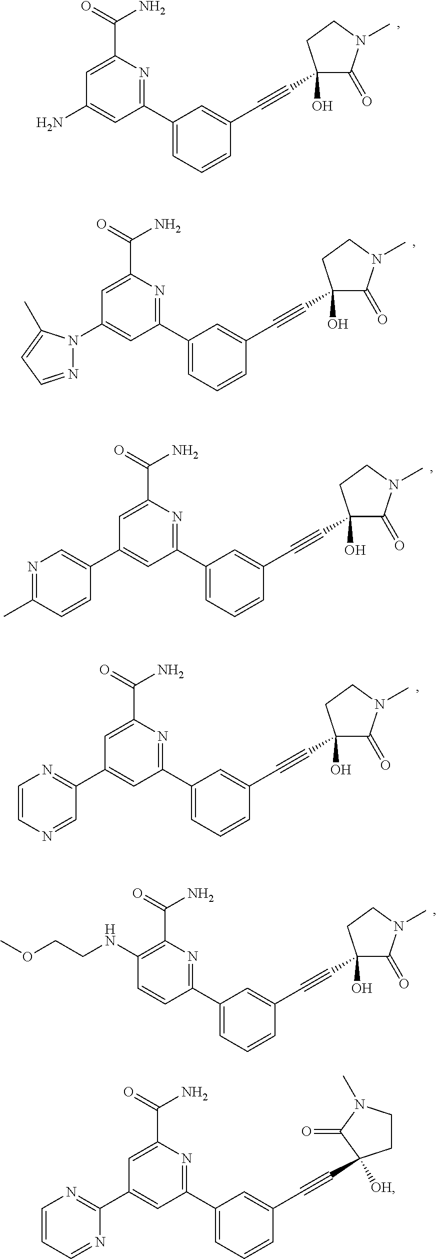 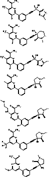 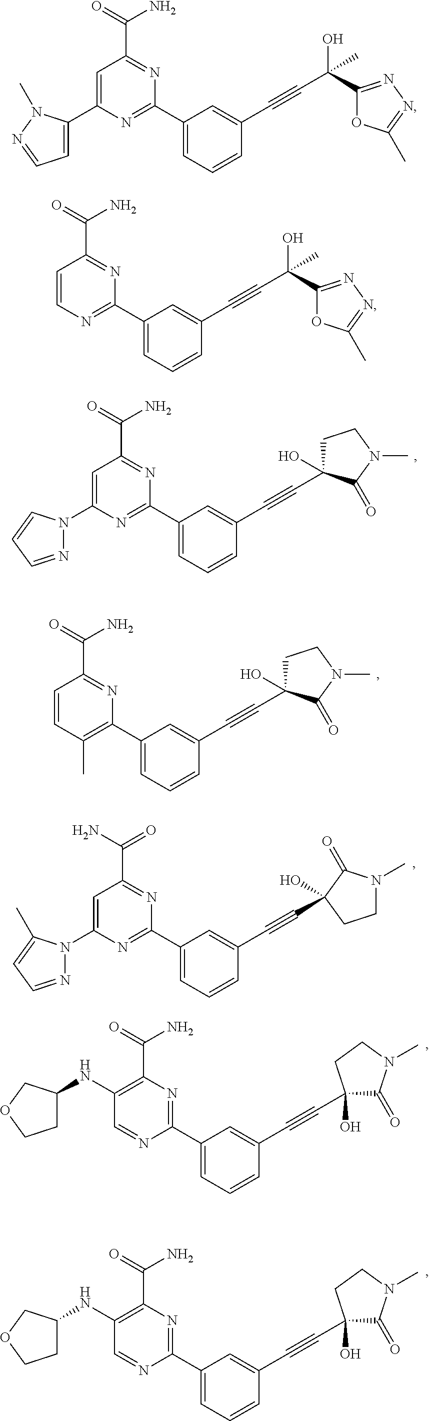 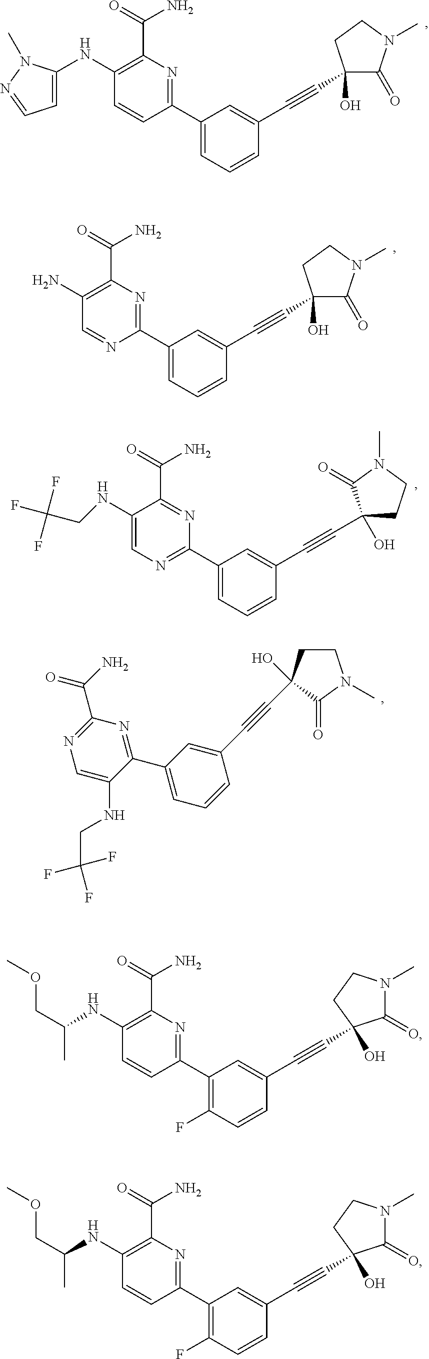 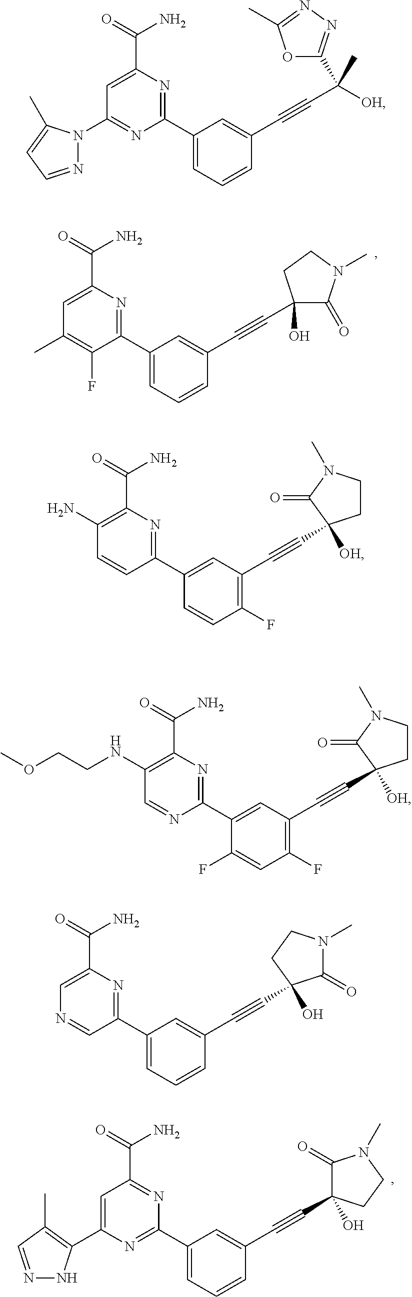 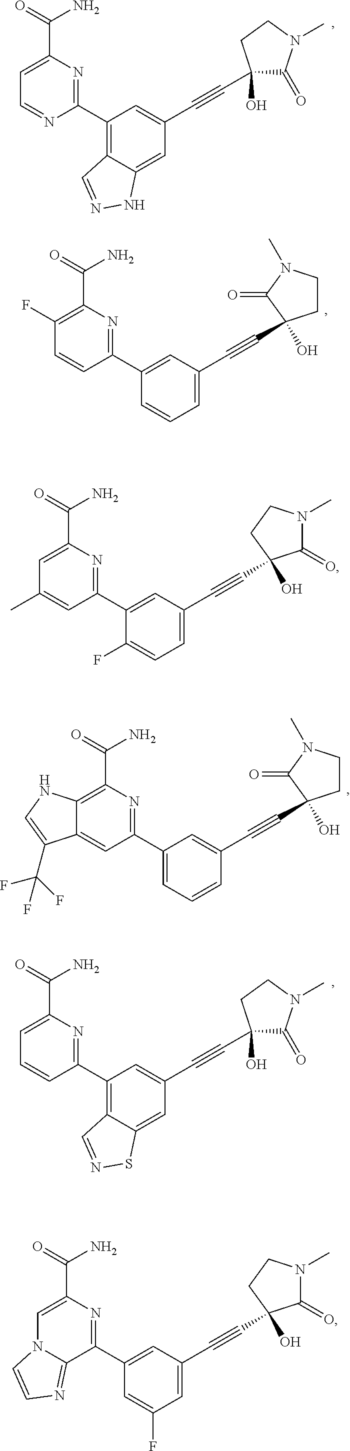 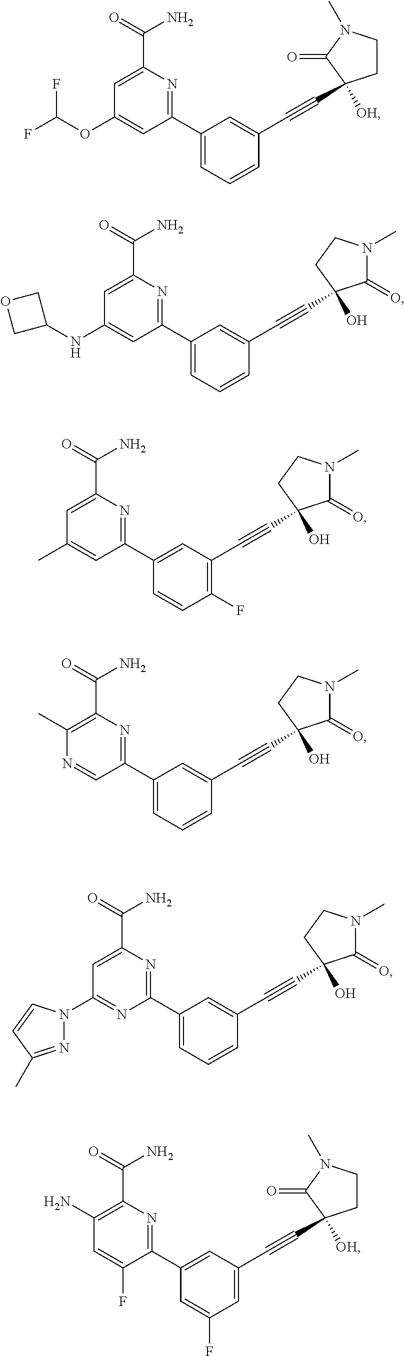 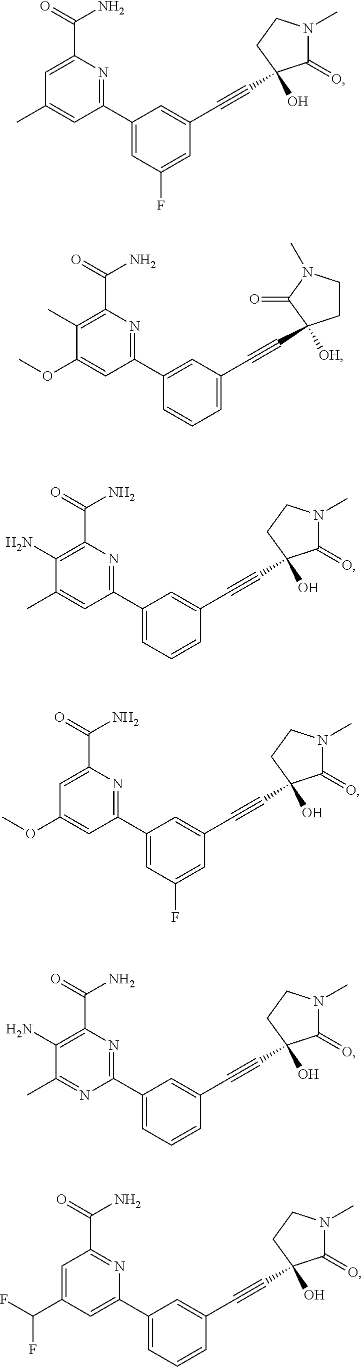 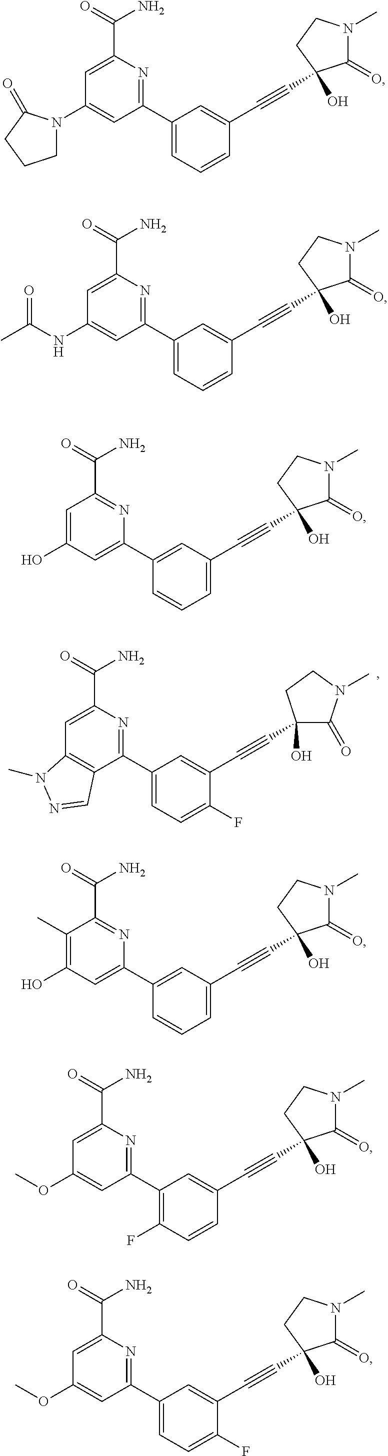 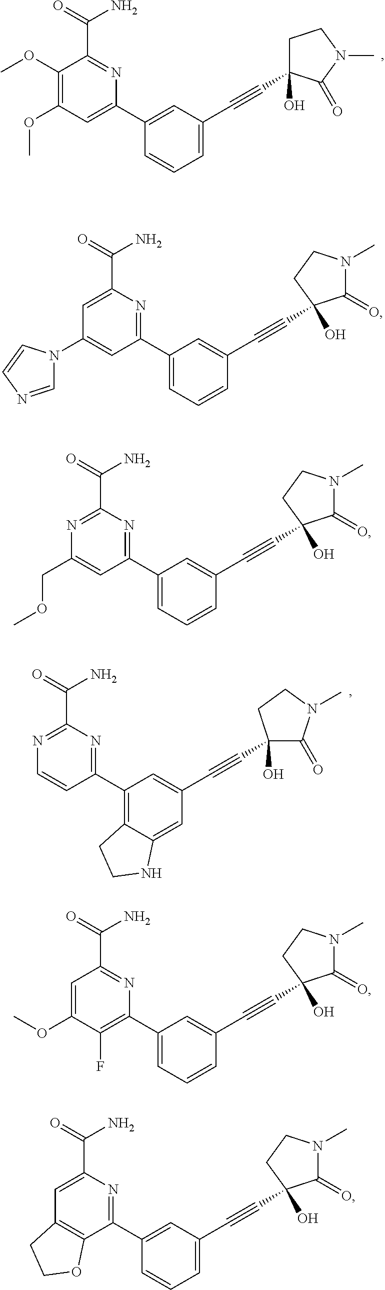 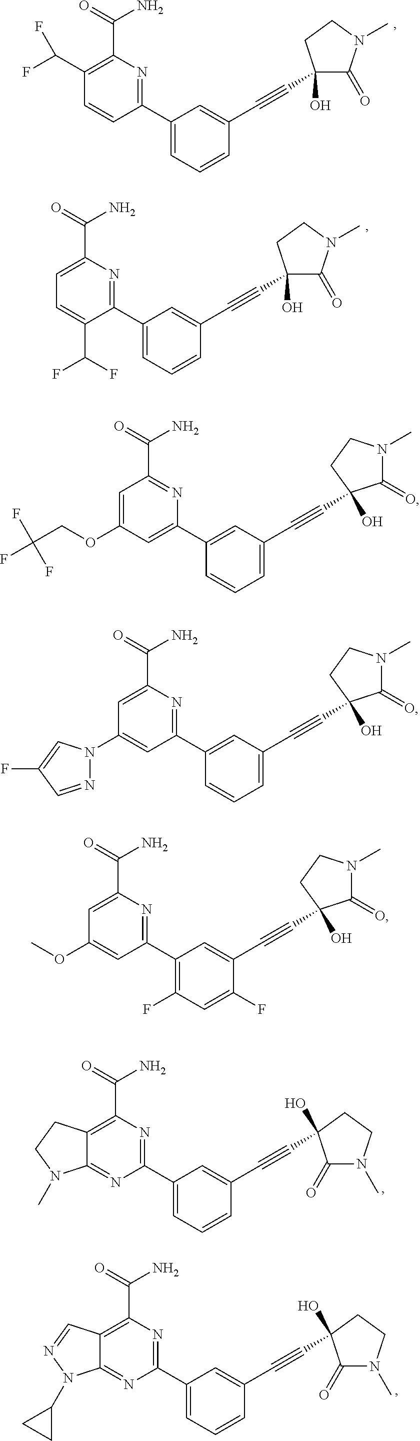 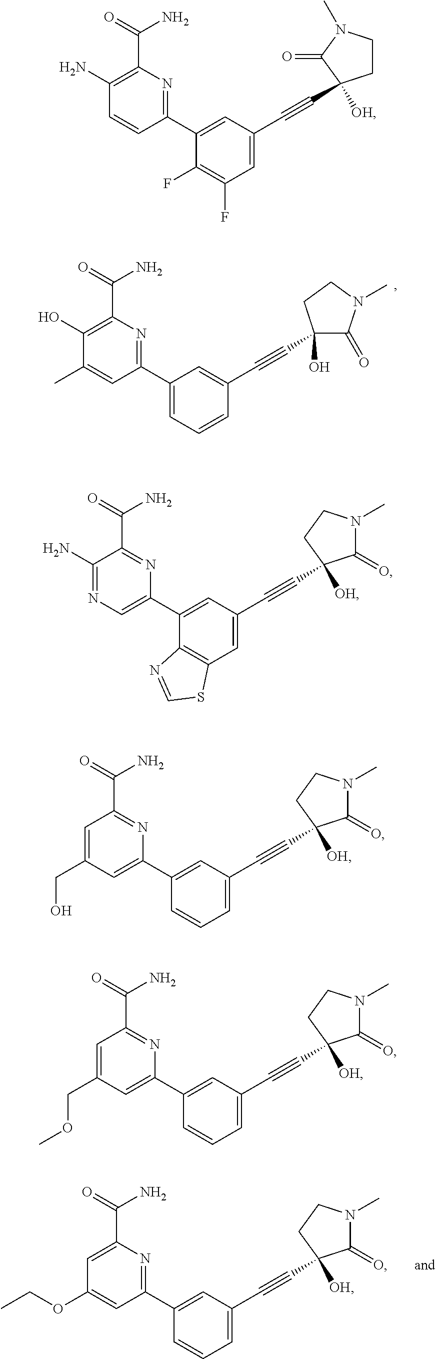 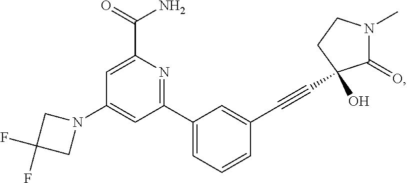 [SMILES: CN1CC[C@@](O)(C#Cc2cccc(-c3cc(N4CC(F)(F)C4)cc(C(N)=O)n3)c2)C1=O]
or a pharmaceutically acceptable salt thereof.

2. A pharmaceutical composition comprising a compound of claim 1 and a pharmaceutically acceptable carrier, diluent or excipient.

3. A method for the treatment of an inflammatory condition in a patient, comprising administering an effective amount of a compound of claim 1 to the patient.

4. The method of claim 3, wherein the inflammatory condition is selected from the group consisting of lupus, systemic lupus erythematosus, COPD, rhinitis, multiple sclerosis, IBD, arthritis, rheumatoid arthritis, dermatitis, endometriosis and transplant rejection.
